(19) United States
(12) Patent Application Publication
(10) Pub. No.: US 2025/0019357 A1
(43) Pub. Date: Jan. 16, 2025

(54) BENZOFURAN SALT MORPHIC FORMS AND MIXTURES FOR THE TREATMENT OF MENTAL DISORDERS OR MENTAL ENHANCEMENT

(71) Applicant: TACTOGEN INC, Palo Alto, CA (US)

(72) Inventors: Matthew J. Baggott, Redwood City, CA (US); Sean Jeffrey Lofthus, San Diego, CA (US); Xanthe Marie Renomeron De Leon, San Diego, CA (US); Aniruddh Singh, San Diego, CA (US); Connor James Hudgins, San Diego, CA (US)

(73) Assignee: TACTOGEN INC, Palo Alto, CA (US)

(21) Appl. No.: 18/737,506

(22) Filed: Jun. 7, 2024

Related U.S. Application Data
(63) Continuation of application No. PCT/US2022/052325, filed on Dec. 8, 2022.
(60) Provisional application No. 63/287,943, filed on Dec. 9, 2021.
(60) Provisional application No. 63/287,443, filed on Dec. 8, 2021.

(51) Int. Cl. C07D 307/79; A61K 31/343

(52) U.S. Cl. CPC C07D 307/79; A61K 31/343

(57) ABSTRACT

Salt morphic forms, morphic salt mixtures, and specified salt mixtures of pharmaceutically active benzofuran compositions are provided for the treatment of mental disorders or for mental enhancement, including for entactogenic therapy. The present invention also includes salt morphic forms, morphic salt mixtures, and specified salt mixtures of benzofuran compounds, compositions thereof, and methods for generally modulating central nervous system activity and treating central nervous system disorders.

### CROSS-REFERENCE TO RELATED APPLICATIONS

This application is a continuation of International Patent Application No. PCT/US2022/052325, filed in the U.S. Receiving Office on Dec. 8, 2022, which claims the benefit of U.S. Provisional Application 63/287,443 filed on Dec. 8, 2021, and U.S. Provisional Application 63/287,943 filed on Dec. 9, 2021. The entirety of each of these applications is hereby incorporated by reference herein for all purposes.

### FIELD OF THE INVENTION

The present invention is in area of pharmaceutically active benzofuran salt morphic forms, morphic salt mixtures, and specified salt mixtures for the treatment of mental disorders or for mental enhancement, including for entactogenic therapy. These morphic forms and salts can be used to modulate central nervous system activity and treat central nervous system disorders.

### BACKGROUND

Central nervous system (CNS) related health problems are a common challenge in society. An estimated 20.6% of U.S. adults (51.5 million people) experienced mental illness in 2019. This includes major depression (7.8% or 19.4 million people), anxiety disorders (19.1% or 48 million people), and posttraumatic stress disorder (PTSD) (3.6% or 9 million people). In addition to mental health challenges, there are other CNS disorders that cause substantial suffering and decreased quality of life. These include traumatic brain injury (TBI) (an estimated 12% of adults or 30 million people in the U.S.), dementias, and headache disorders (such as migraine, which affects about 15% of the general population or 47 million people in the U.S.). As the global population ages, many age-related CNS disorders are projected to become more common. For example, 6.2 million people aged 65 and older in the U.S. have Alzheimer's dementia and this population is expected to grow to 12.7 million by 2050.

There is a need for improved treatment of CNS disorders. Many patients fail to benefit adequately from available treatments. In addition, many available pharmacological treatments must be taken for weeks or months before the individual experiences therapeutic benefits. Because of these and other considerations, fewer than half of U.S. adults with mental illness (44.8%) received treatment in 2019.

A number of potential new experimental treatments are under investigation. These include novel compounds that modulate the functioning of the monoamine neurotransmitters, dopamine, norepinephrine, and serotonin. Dopamine is involved in learning, incentives, and the initiation of motor movements. Norepinephrine is important for attention and cardiovascular functioning.

Serotonin is incompletely understood but appears to adjust the stability of the individual's response to changing environmental conditions. As such, serotonin has been linked to mood, anxiety, and appetite.

New experimental treatment compounds include serotonin receptor agonists. Serotonin receptors have seven families, and many receptors are able to stimulate multiple signaling pathways within a cell, which can make it complicated to predict therapeutic effects. Serotonin receptor types that have received recent attention for their therapeutic potential include 5-HT_{2A}, 5-HT_{2C}, 5-HT_{1A}, and 5-HT_{1B} receptors.

One group of experimental therapeutic compounds are 5-HT_{2A} receptor agonists. These are being investigated as tools for producing rapid therapeutic improvement in CNS disorders including depression, anxiety, and substance use disorders. Many, such as psilocybin and 5-methoxy-N,N-dimethyltryptamine (5-MeO-DMT), produce dramatic psychedelic effects resembling mystical experiences that may contribute to these therapeutic effects. These compounds also produce labile mood and often invoke acute anxiety, which makes close monitoring of patients necessary. There is accordingly a need for 5-HT_{2A} agonists that produce either minimal mood changes or reliably positive ones.

Another group of putative 5-HT_{2A} agonists, such as 6-methoxy-N,N-dimethyltryptamine (6-MeO-DMT) and 7-fluoro-N,N-dimethyltryptamine (7-F-DMT) appear to produce therapeutic changes in animal models of depression without producing psychedelic effects (Dunlap et al. 2020. Journal of medicinal chemistry, 63(3), pp. 1142-1155). Both psychedelic and non-psychedelic 5-HT_{2A} agonists may be useful in migraine, cluster headaches, and other headache disorders.

The therapeutic mechanisms of 5-HT_{2A} agonists are incompletely understood but may involve increased neuroplasticity (Ly et al. 2018. Cell reports, 23(11), pp. 3170-3182), suggesting potential benefits in TBI, neurological disorders, and conditions where behavior change or learning is desired.

Another potential therapeutic mechanism of 5-HT_{2A} agonists involves decreases in inflammation (e g., Flanagan, et al. 2019. Life sci., 236, 116790). Conditions that may benefit from improved anti-inflammatory treatment include rheumatoid and other forms of arthritis (such as enthesitis-related juvenile idiopathic arthritis, blau syndrome, and juvenile idiopathic arthritis), psoriasis, Crohn's disease, inflammatory bowel syndrome, ulcerative colitis, and ankylosing spondylitis. Inflammation has long been recognized to induce symptoms of depression (Lee & Giuliani. 2019. Frontiers in immunology, 10, 1696). Inflammatory processes have also been implicated in psychotic disorders (Borovcanin et al. 2012. J. Psych. Res., 46(11), 1421-1426) and bipolar disorders (Hamdani, Tamouza, & Leboyer. 2012. Front. Biosci. (Elite Ed.), 4, 2170-2182).

5-HT_{2A} agonists are also often 5-HT₂B agonists. This is undesirable because chronic stimulation of 5-HT₂B receptors causes cardiac valvulopathy (Rothman et al. 2000. Circulation, 102(23), pp. 2836-2841). There is therefore a need for serotonin agonists that have decreased ability to stimulate 5-HT₂B receptors.

5-HT_{2C} receptors are closely related to 5-HT_{2A} receptors, but have a different distribution in the brain and body. Compounds that stimulate 5-HT_{2C} receptors have been proposed as treatments for psychiatric disorders as well as other disorders such as sexual dysfunction, obesity, and urinary incontinence. Lorcaserin (Belviq) is a high affinity 5-HT_{2C} agonist that, until recently, was FDA-approved for use in conjunction with weight loss programs. The withdrawal of this medicine from the market because of increased risk of cancer highlights the need for safer serotonergic therapeutics that can stimulate 5-HT_{2C} receptors or otherwise aid weight loss.

5-HT_{1A} receptor agonists modulate the functioning of dopamine and norepinephrine and decrease blood pressure and heart rate via a central mechanism. Drugs that are 5-HT_{1A} agonists have value for treating anxiety and depression. For example, buspirone (Buspar, Namanspin) is approved for anxiety disorders and may also be useful for treating hypoactive sexual desire disorder (HSDD). Studies in rats indicate that 5-HT_{1A} stimulation induces oxytocin release, which contributes to the social effects of 3,4-methylenedioxymethamphetamine (MDMA) (Thompson et al. 2007. Neuroscience, 146(2), pp. 509-514). Compounds (or compound combinations) that include 5-HT_{1A} stimulation in their pharmacological profile are therefore expected to have therapeutic benefits in comparison to those that do not.

Compounds that stimulate 5-HT_{1B} receptors alter the release of neurotransmitters such as dopamine, serotonin, GABA, acetylcholine, and glutamate and can modulate stress sensitivity, mood, anxiety, and aggression. 5-HT_{1B} agonists such as sumatriptan (Imitrex) and zolmitriptan (Zomig) have been approved for treatment of headache disorders. Studies in mice suggest 5-HT_{1B} stimulation on dopamine-containing neurons in the central striatum contributes to social effects of MDMA (Heifets et al. 2019. Science translational medicine, 11(522)). Preclinical studies also suggest 5-HT_{1B} agonists may have antidepressant effects. More broadly, there is evidence that stimulating 5-HT_{1B} receptors can provide benefits to stress response, affect, and addiction (e.g., Fontaine et al. 2021. Neuropsychopharmacology, pp. 1-11). As with 5-HT_{1A} receptors, compounds (or compound combinations) that include 5-HT_{1B} stimulation in their pharmacological profile are therefore expected to have therapeutic benefits in comparison to those that do not.

Another group of experimental compounds interact with brain monoamine transporters to increase extracellular concentrations of the three monoamine neurotransmitters. This allows stimulation of multiple receptor types by the neurotransmitter. Some compounds increase extracellular concentrations of these molecules by inhibiting reuptake of neurotransmitters, while others induce release of neurotransmitters. Inhibition of reuptake will disproportionately affect active synapses where neurotransmitter release has taken place, while release of monoamine neurotransmitter occurs independently from which synapses are active. Release can also produce greater extracellular increases than inhibiting uptake. While greater increases in neurotransmitter can produce greater (and, in some cases, faster onset of) therapeutic effects, high and prolonged concentrations of releasers can also cause metabolic stress within monoaminergic neurons, potentially leading to neurotoxicity. When the neurotransmitter in question is dopamine, large extracellular increases are additionally associated with abuse liability and risk of addiction.

Nicotine and other nicotinic receptor agonists and antagonists have been reported to potentiate antidepressant effects in rodents (Popik et al. 2003, Br. J. Pharmacol, 139, 1196-1202; Andreasen et al., 2011, J. Psychopharm. 25(10), 1347-56). Clinical and preclinical findings point to an association between nicotinic acetylcholine receptors (nAChRs), especially the α4β2 subtype, and depression, with a number of α4β2 nAChR ligands showing antidepressant-like effects in rodent screening tests, such as the forced swim test (reviewed in Yu et al. 2014, J. Med. Chem, 57(20), 8204-23). However, advances in the understanding of cholinergic and nicotinic neurotransmission have not yet resulted in significant progress in the clinical treatment of CNS disorders.

Patent applications describing entactogenic compounds include WO 2021/252538, WO 2022/010937, WO 2022/032147, and WO 2022/061242 which are assigned to Tactogen Inc. Additional patent applications describing entactogenic compounds and methods of using entactogenic compounds include but are not limited to U.S. Pat. No. 7,045,545, WO 2005/058865, WO 2020/169850, WO 2020/169851, WO 2021/257169, WO 2021/225796, WO 2022/214889, WO 2022/120181, WO 2022/072808, and WO 2022/038171.

Despite the ongoing research on potential new drugs to treat mental disorders, CNS disorders, and related gastrointestinal and inflammatory disorders, the large burden of disease caused by these disorders remains a global serious and systemic problem. New drugs and treatments are required to improve personal well-being, mental health, and physical health that are dependent on the alteration of neurotransmitter levels and performance.

It is therefore an object of the present invention to provide advantageous compositions and their use and manufacture for the treatment of mental disorders and enhancement for hosts, typically humans, in need thereof. Additional objects are to provide drugs with an efficient onset to be used in a clinical setting such as counseling or a home setting, which open the patient to empathy, sympathy, and acceptance. A further object is to provide effective treatments for a range of CNS disorders.

### SUMMARY OF THE INVENTION

The present invention provides advantageous salt morphic forms, morphic salt mixtures, and specified salt mixtures as described herein of benzofuran compounds to treat mental disorders and more generally central nervous system and related disorders as described herein. A benzofuran salt morphic form, morphic salt mixture, or specified salt mixture of the present invention can be used for mental enhancement or to treat a mental disorder comprising administering an effective amount of the benzofuran salt morphic form, morphic salt mixture, or specified salt mixture as described herein to a host, typically a human, in need thereof. In some embodiments, the benzofuran salt morphic forms or compositions described herein interact with a serotonergic binding site and can exhibit entactogenic properties when administered in an effective amount to a host, typically a human, in need thereof. Thus, a benzofuran salt morphic form, morphic salt mixture, or specified salt mixture as described herein can be used as an effective agent for modulating CNS activity and treating CNS disorders described herein.

In certain aspects salt morphic form, morphic salt mixture, or specified salt mixture described herein of R-5-MAPB, S-5-MAPB, R-6-MAPB, S-6-MAPB, R-Bk-5-MAPB, S-Bk-5-MAPB, R-Bk-6-MAPB, or S-Bk-6-MAPB or an enantiomerically enriched mixture thereof is provided. In other aspects a salt morphic form, morphic salt mixture, or specified salt mixture described herein of R/S-5-MAPB, R/S-6-MAPB, R/S-Bk-5-MAPB, or R/S-Bk-6-MAPB is provided.

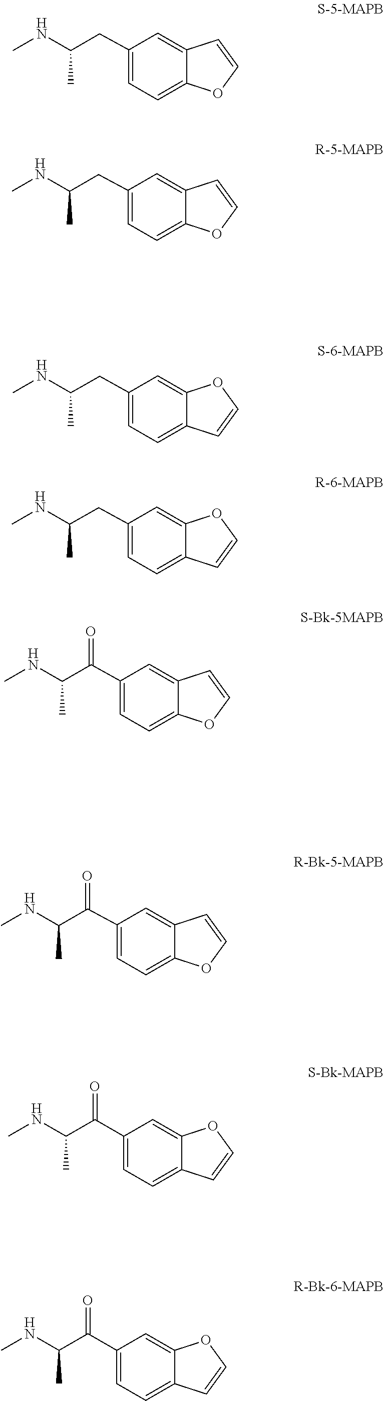

In other aspects a salt morphic form, morphic salt mixture, or specified salt mixture described herein of R-5-MBPB, S-5-MBPB, R-6-MBPB, S-6-MBPB, R/S-5-MBPB, or R/S-6-MBPB is provided.

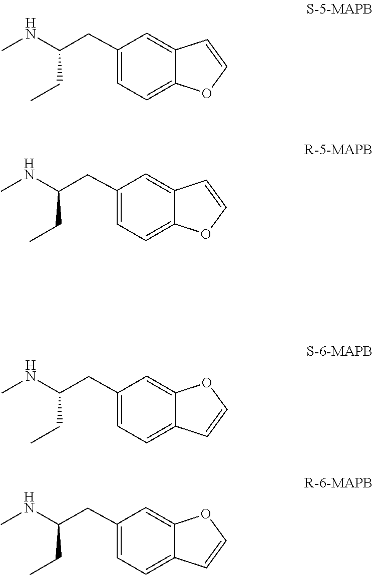

The selection of a salt morphic form, morphic salt mixture, or specified salt mixture can increase desired manufacturing and/or pharmacokinetic properties. In certain embodiments the selected salt decreases undesirable manufacturing properties, pharmacokinetic properties, and/or side effects. For example, in certain embodiments one salt form will be absorbed faster in a desired organ (for example the intestine) than another (see Example 25 showing faster predicted absorption of S-5-MAPB HCl than of S-5-MAPB oxalate). When the therapeutic indication requires a faster onset of medicinal effects the salt that is more quickly absorbed may be superior to the less quickly absorbed salt. When the therapeutic indication requires a slower onset of medicinal effects the salt that is more slowly absorbed may be superior to the quickly absorbed salt. In certain aspects a mixture of salts can be administered to provide a quick onset of medicinal effect with a prolonged duration. For example, in certain embodiments a mixture of S-5-MAPB HCl and S-5-MAPB oxalate is administered to a patient.

Additional examples of therapeutic properties that can be improved with a salt morphic form or a mixture of salts of a benzofuran compound described herein include: increased dissolution or absorption, targeted drug delivery, improved taste, reduced pain on injection (for intravenous formulations), improved taste (for oral formulations), improved drug effectiveness, increased Cmax, increased exposure, and increased half-life. Salts can also be selected to decrease these properties, for example in certain contexts decreasing the Cmax or half-life of a compound is advantageous for therapeutic use.

Additional examples of manufacturing properties that can be improved with a salt morphic form or a mixture of salts of a benzofuran compound described herein include: ease of processing (for example increased flowability, improved rolling properties, improved pouring properties, or less clumping), decreased hydrophobicity, increased solubility, increased stability, increased purity, or increased or decreased particle size.

The invention also provides advantageous morphic forms of R-5-MAPB, S-5-MAPB, R/S-5-MAPB, and S-6-MAPB salts. These morphic forms provided important starting materials and intermediates in the manufacture of R-5-MAPB, S-5-MAPB, R/S-MAPB, and S-6-MAPB for medicinal use and can increase desired manufacturing and/or pharmacokinetic properties while decreasing undesirable manufacturing properties, pharmacokinetic properties, and/or side effects.

Morphic forms of RS-5-MAPB HCl, RS-5-MAPB HBr, RS-5-MAPB H₃PO₄, RS-5-MAPB oxalic acid, RS-5-MAPB maleic acid, S-5-MAPB HCl, S-5-MAPB HBr, S-5-MAPB H₃PO₄, S-5-MAPB oxalic acid, S-5-MAPB fumaric acid, R-5-MAPB HCl, S-6-MAPB HCl, S-6 MAPB HBr, S-6-MAPB H₃PO₄, and S-6-MAPB oxalic acid are provided.

Advantageous treatments for CNS disorders and methods to provide mental enhancement are provided that use a selected salt morphic form or a mixture of salts of a compound described herein. The properties of these compounds can be further enhanced by using an enantiomerically enriched mixtures or single enantiomer of a benzofuran compound. For example, mixtures that have a greater amount of the S-enantiomer 5-MAPB or 6-MAPB maximize serotonin-receptor-dependent therapeutic effects, and that enantiomerically enriched mixtures that have a greater amount of R-enantiomer of 5-MAPB or 6-MAPB maximize nicotinic-receptor-dependent therapeutic effects. Therefore, one aspect of the present invention is an enantiomerically enriched mixture of a compound as a salt morphic form, morphic salt mixture, or specified salt mixture described herein for example S-5-MAPB and R-5-MAPB or an enantiomerically enriched mixture of S-6-MAPB and R-6-MAPB, that achieves a combination of serotonin-receptor-dependent therapeutic effects and nicotinic-receptor-dependent or dopaminergic therapeutic effects. The effect can be modulated as desired for optimal therapeutic effect.

Accordingly, in one embodiment, an enantiomerically enriched mixture of an S-5-MAPB salt morphic form, morphic salt mixture, or specified salt mixture described herein or an enantiomerically enriched mixture of S-6-MAPB salt morphic form, morphic salt mixture, or specified salt mixture described herein, maximizes serotonin-receptor-dependent therapeutic effects and minimize unwanted nicotinic effects or dopaminergic effects when administered to a host in need thereof, for example a mammal, including a human.

In another embodiment, an enantiomerically enriched mixture of R-5-MAPB salt morphic form, morphic salt mixture, or specified salt mixture described herein or an enantiomerically enriched mixture of R-6-MAPB salt morphic form, morphic salt mixture, or specified salt mixture described herein, maximizes nicotinic-receptor-dependent or dopaminergic-receptor dependent therapeutic effects while minimizing unwanted effects, when administered to a host in need thereof, including a mammal, for example, a human.

Enantiomerically enriched mixtures of 5-MAPB that are non-racemic have a relatively greater amount of some therapeutic effects (such as emotional openness) while having lesser effects associated with abuse liability (such as perceptible ‘good drug effects’). Additionally, any such abuse liability would be expected to be attenuated to the extent that the substance also increases extracellular serotonin (see, e.g., Wee et al., Journal of Pharmacology and Experimental Therapeutics, 2005, 313(2), 848-854). Therefore, one aspect of the present invention is an enantiomerically enriched mixture of a S-5-MAPB salt and a R-5-MAPB salt morphic form, morphic salt mixture, or specified salt mixture thereof or an enantiomerically enriched mixture of a S-6-MAPB salt and a R-6-MAPB salt morphic form, morphic salt mixture, or specified salt mixture thereof that achieves a predetermined combination of emotional therapeutic effects and perceptible mood effects. The effect can be modulated as desired for optimal therapeutic effect.

While all enantiomers of 5-MBPB and 6-MBPB are full releasers of serotonin, R-5-MBPB and R-6-MBPB are apparently partial releasers of norepinephrine and reuptake inhibitors of dopamine and S-6-MBPB is a partial releaser of both dopamine and norepinephrine. Partial releasers are molecules that produce limited increases in neurotransmitter (i.e., Emax less than 100%). They are thought to cause either partial blockage of the translocation pathway in the monoaminergic transporter (due to long dwell time or a docking pose that prevents transport) or stabilization of an inactive or inward-facing conformation of the transporter (e.g., Hasenhuetl et al. 2019. Molecular Pharmacology, 95(3), pp.303-312; Niello et al. 2019. Neuropharmacology, 161, p.107615; Rothman et al. 2012. Journal of Pharmacology and Experimental Therapeutics, 341(1), pp.251-262). Reuptake inhibitors act on active monoaminergic synapses while releasers act on all monoaminergic synapses, with different ratios of release and inhibition producing different effects. Therefore, different mixtures of the S- and R-enantiomers of these molecules produce different maximum effects on norepinephrine and dopamine that are lesser than those produced by a full releaser such as MDMA. The limited increases in dopamine and the higher Emax for serotonin produced by these mixtures limits the euphoria and abuse liability produced after higher doses of these mixtures. Specifically, the DAT to SERT ratio decreases in a concentration-dependent manner, causing higher doses and concentrations to have less abuse liability than lower doses. Because dose escalation is a characteristic of addiction and substance use disorders, the relatively greater serotonergic and lesser dopaminergic nature of higher doses is expected to protect against abuse. The limited increases in norepinephrine produced by these mixtures similarly limits the cardiovascular effects produced after higher doses of these mixtures. Therefore, one aspect of the present invention is an enantiomerically enriched mixture of a compound as a salt morphic form, morphic salt mixture, or specified salt mixture described herein, for example S-5-MBPB and R-5-MBPB or an enantiomerically enriched mixture of S-6-MBPB and R-6-MBPB, that achieves a combination of serotonin-receptor-dependent therapeutic effects and norepinephrine-receptor-dependent and dopaminergic-receptor-dependent therapeutic effects, while having reduced euphoria and abuse liability and reduced cardiovascular effects. The effect can be modulated as desired for optimal therapeutic effect.

The choice of salt morphic form, morphic salt mixture, or specified salt mixture can further enhance these beneficial effects. For example, a salt morphic form, morphic salt mixture, or specified salt mixture described herein can have beneficial effects on the pharmacokinetic or pharmacodynamic properties of the compound. These effects include increased or decreased bioavailability, absorption, half-life, peak exposure, total exposure, and/or other properties. Increasing or decreasing one or more of these properties can be beneficial for different applications of the benzofuran compound to treat CNS disorders or provide mental enhancement.

In certain aspects, the present invention provides a salt morphic form, morphic salt mixture, or specified salt mixture described herein of an enantiomer, or enantiomerically enriched mixture of Formula A, Formula B, Formula C, Formula D, Formula E, or Formula F:

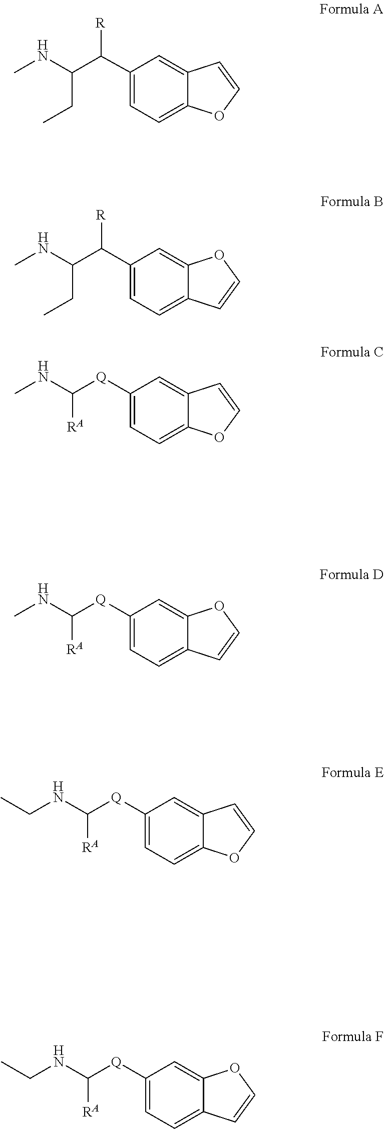

wherein
R is hydrogen or hydroxyl.
R^{A} is —CH₃, —CH₂Y, —CHY₂, —CY₃, —CH₂CH₃, —CH₂CH₂Y, —CH₂CHY₂, —CH₂CY₃, —CH₂OH, or —CH₂CH₂OH;
Q is selected from:

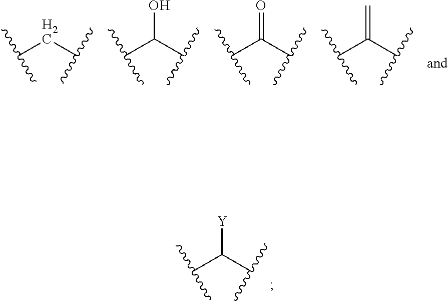

Y is halogen.

Non-limiting examples of compounds of Formula C and Formula D include 5-MBPB, 6-MBPB, Bk-5-MAPB and Bk-6-MAPB:

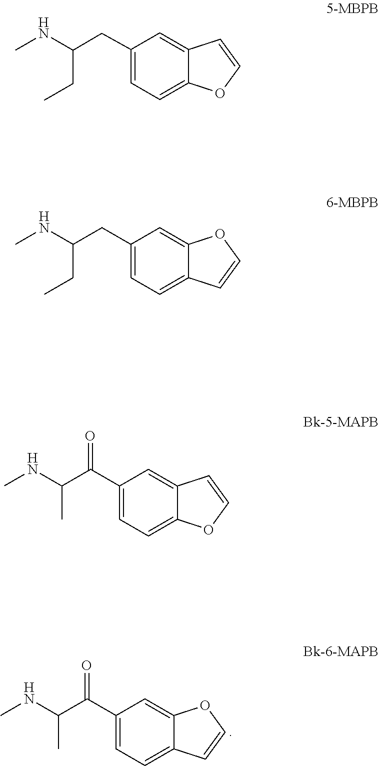

In other embodiments, the invention provides a salt morphic form, morphic salt mixture, or specified salt mixture described herein of a compound, enantiomer, or enantiomerically enriched mixture of Formula I, Formula II, Formula III, Formula IV, Formula V, Formula VI, Formula VII, Formula VIII, Formula IX, or Formula X:

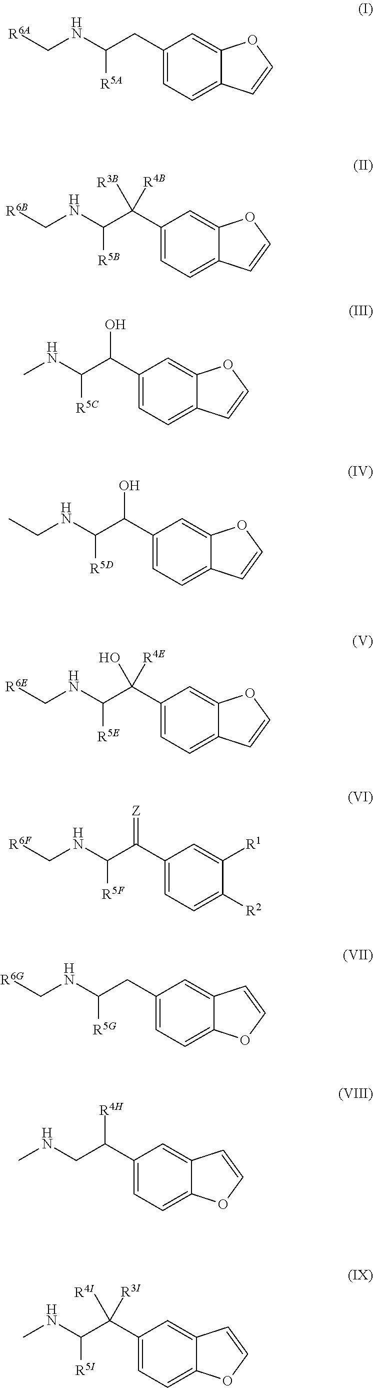

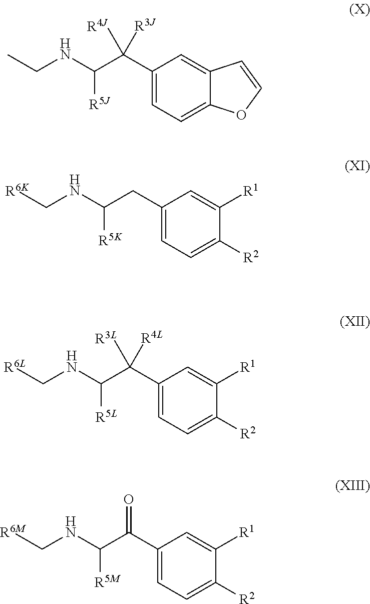

wherein:
   R¹ and R² are taken together as —OCH═CH— or —CH═CHO—;
   R^{3B} and R^{4B} are independently selected from —H, —X, C₁-C₄ alkyl, —CH₂OH, —CH₂X, —CHX₂, and —CX₃, wherein at least one of R^{3B} and R^{4B} is not —H;
   R^{3L} and R^{4L} are independently selected from —H, —X, —OH, C₁-C₄ alkyl, —CH₂OH, —CH₂X, —CHX₂, and —CX₃, wherein at least one of R^{3L} and R^{4L} is not —H;
   R^{3I} and R^{4I} are independently selected from —H, —X, —OH, —CH₂OH, —CH₂X, —CHX₂, —CX₃, and C₁-C₄ alkyl; wherein at least one of R^{3I} and R^{4I} is not —H;
   R^{3J} and R^{4J} are independently selected from —H, —X, —OH, C₁-C₄ alkyl, —CH₂OH, —CH₂X, —CHX₂, and —CX₃;
   R^{4E} is selected from C₁-C₄ alkyl, —CH₂OH, —CH₂X, —CHX₂, and —CX₃; R^{4H} is selected from —X, —CH₂CH₂CH₃, —CH₂OH, —CH₂X, and —CHX₂;
   R^{5A} and R^{5G} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl, when R^{5A} is C₂ alkyl or H, R^{6A} is not —H, and when R^{5G} is —H or C₂ alkyl, R^{6G} is not —H;
   R^{5B} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl;
   R^{5C} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
   R^{5D}, R^{5E}, R^{5F}, and R^{5J} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl, when R^{5F} is —H or C₁ alkyl, R^{6F} cannot be —H, and when R^{5J} is C₁ alkyl, at least one of R^{3J} and R^{4J} is not H;
   R^{5K} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
   R^{5L} and R^{5M} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; and
   R^{5I} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; wherein at least one of R^{3I}, R^{4I}, and R^{5I} is not C₁ alkyl;
   R^{6A}, R^{6B}, R^{6E}, R^{6F}, and R^{6G} are independently selected from —H and —CH₃;
   R^{6K}, R^{6L}, and R^{6M} are independently selected from —H and —CH₃;
   X is independently selected from —F, —Cl, and —Br; and
   Z is selected from O and CH₂.

In certain embodiments, a salt morphic form, morphic salt mixture, or specified salt mixture described herein of a compound of Formulas I-XIII is used as described herein in enantiomerically enriched form to achieve the goals of the invention. In other embodiments, a salt morphic form, morphic salt mixture, or specified salt mixture described herein of the compound is used as a racemate or a pure enantiomer, for example a substantially pure enantiomer. A substantially pure enantiomer has an enantiomeric purity of at least about 98%. In certain embodiments a substantially pure enantiomer is at least 98% and less than 100% enantiomerically pure.

The invention additionally includes methods to treat a neurological or psychiatric central nervous system disorder as further described herein, including a mental disorder, or to provide a mental enhancement, with salt morphic form, morphic salt mixture, or specified salt mixture described herein of a compound of Formula A, Formula B, Formula C, Formula D, Formula E, Formula F, Formula I, Formula II, Formula III, Formula IV, Formula V, Formula VI, Formula VII, Formula VIII, Formula IX, Formula X, Formula XI, Formula XII, or Formula XIII or a salt morphic form of R-5-MAPB, S-5-MAPB, R/S-5-MAPB, or S-6-MAPB.

In certain embodiments, a selected salt morphic form, morphic salt mixture, or specified salt mixture of the present invention is administered to a human patient in an effective amount in conjunction with psychotherapy, cognitive enhancement, or life coaching (pharmacotherapy), or as part of routine medical therapy.

Nonlimiting examples of salts include HCl, sulfate, aspartate, saccharate, phosphate, oxalate, acetate, gluconate, maleate, malate, citrate, mesylate, nitrate, tartrate, amino acid anion, benzenesulfonate, benzoate, besylate, bicarbonate, bitartrate, camsylate, carbonate, cdecanoate, edetate, esylate, fumarate, gluceptate, cluconate, clutamate, glycolate, hexanoate, hydroxynapthtoate, HI, isethionate, lactate, lactobionate, mandelate, methylsulfate, mucate, napsylate, octanoate, oleate, pamoate, pantothenate, phosphate, polycalacturonate, propionate, salicylate, stearate, sulfate, teoclate, tosylate, or a mixture thereof.

In certain embodiments a benzofuran compound described herein is provided as a salt mixture wherein the salt mixture comprises HCl and at least one additional salt selected from HBr, H₂SO₄, H₃PO₄, HNO₃, methanesulfonic, succinic, oxalic, maleic, fumaric, saccharate, aspartate, L-Arginine, and L-Lysine. For example, a benzofuran compound described herein as a mixture of HCl and oxalate salt.

The present invention thus includes at least the following aspects:
(i) A salt morphic form, morphic salt mixture, or specified salt mixture described herein of R-5-MAPB, S-5-MAPB, R/S-5-MAPB, S-6-MAPB, 5-MAPB, 6-MAPB, 5-MBPB, 6-MBPB, Bk-5-MAPB, Bk-6-MAPB, Formula I, Formula II, Formula III, Formula IV, Formula V, Formula VI, Formula VII, Formula VIII, Formula IX, Formula X, Formula XI, Formula XII, Formula XIII, Formula A, Formula B, Formula C, Formula D, Formula E, or Formula F or an isotopic derivative, or prodrug thereof;
(ii) A pharmaceutical composition comprising an effective patient-treating amount of a salt morphic form, morphic salt mixture, or specified salt mixture of a benzofuran described herein with a pharmaceutically acceptable carrier or diluent;
(iii) The pharmaceutically acceptable composition of (ii) in a solid dosage form;
(iv) The pharmaceutically acceptable composition of (ii) in a liquid dosage form;
(v) The pharmaceutically acceptable composition of (ii), (iii), or (iv) which is suitable for systemic delivery;
(vi) The pharmaceutically acceptable composition of (ii), (iii), or (iv) which is suitable for oral delivery;
(vii) The pharmaceutically acceptable composition of (ii), (iii), or (iv) which is suitable for topical delivery;
(viii) The pharmaceutically acceptable composition of (ii), (iii), or (iv) which is suitable for parental delivery;
(ix) A method for treating a patient with any neurological or psychological CNS disorder as described herein that includes administering an effective amount of salt morphic form, morphic salt mixture, or specified salt mixture of (i) to a patient such as a human in need thereof;
(x) A method for treating PTSD, depression, dysthymia, anxiety, generalized anxiety, social anxiety, panic, adjustment disorder, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism or dissociative disorders comprising administering an effective amount of salt morphic form, morphic salt mixture, or specified salt mixture form of (i) or a isotopic derivative, or prodrug thereof, as described herein, to a patient, typically a human, in need thereof;
(xi) A salt morphic form, morphic salt mixture, or specified salt mixture of (i) or an isotopic derivative, or prodrug thereof, for use to treat any disorder as described herein in an effective amount as further described herein;
(xii) A salt morphic form, morphic salt mixture, or specified salt mixture of (i) for use in the manufacture of a medicament for the treatment of any of the disorders described herein;
(xiii) Use of a salt morphic form, morphic salt mixture, or specified salt mixture of (i) or an isotopic derivative, or prodrug thereof, to treat any disorder described herein in an effective amount as further described herein;
(xiv) Processes for the preparation of therapeutic products that contain an effective amount of a salt morphic form, morphic salt mixture, or specified salt mixture of (i) or an isotopic derivative, or prodrugs thereof, as described herein.

### BRIEF DESCRIPTION OF THE FIGURES

FIG. 1 provides the structures and names of several compounds referred to herein.

FIG. 2 is a chart showing results from the marble burying assay to measure decreased anxiety and neuroticism resulting from treatment with S-5-MAPB, R/S-5-MAPB, and R-5-MAPB. The x-axis of the chart displays anxiolytic effect, described as the percent of marbles left unburied versus placebo. The y-axis gives the compound and dose. Error bars indicate 95% confidence intervals. Details and procedural information for this assay are described in Example 5.

FIG. 3 is a chart showing results from the marble burying assay to measure decreased anxiety and neuroticism resulting from treatment with S-6-MAPB, RS-6-MAPB, and R-6-MAPB. The x-axis of the chart displays anxiolytic effect, described as the percent of marbles left unburied versus placebo. The y-axis gives the compound and dose. Error bars indicate 95% confidence intervals. Details and procedural information for this assay are described in Example 5.

FIG. 4 is a chart showing results from the marble burying assay to measure decreased anxiety and neuroticism resulting from treatment with (+)-Bk-5-MAPB, RS-Bk-5-MAPB, and (−)-Bk-R-5-MAPB. The x-axis of the chart displays anxiolytic effect, described as the percent of marbles left unburied versus placebo. The y-axis gives the compound and dose. Error bars indicate 95% confidence intervals. Details and procedural information for this assay are described in Example 5.

FIG. 5 is a chart showing results from the marble burying assay to measure decreased anxiety and neuroticism resulting from treatment with (+)-Bk-5-MBPB, RS-Bk-5-MBPB, and (−)-Bk-R-5-MBPB. The x-axis of the chart displays anxiolytic effect, described as the percent of marbles left unburied versus placebo. The y-axis gives the compound and dose. Error bars indicate 95% confidence intervals. Details and procedural information for this assay are described in Example 5.

FIG. 6 is a chart showing results from the marble burying assay to measure decreased anxiety and neuroticism resulting from treatment with individual enantiomers of 5-MAPB vs the racemic mixture, demonstrating the non-additive effects of the two enantiomers. The x-axis of the chart displays anxiolytic effect, described as the percent of marbles left unburied versus placebo. The y-axis gives the compound and dose. Error bars indicate 95% confidence intervals. Details and procedural information for this assay are described in Example 5.

FIG. 7A is a graph showing results from an in vitro rat synaptosome serotonin uptake inhibition assay. The graphs display percent reuptake of [³H]-labeled 5-HT as a function of concentration for RS-5-MBPB, R-5-MBPB, and S-5-MBPB. This data indicates that each tested compound rapidly increases extracellular serotonin by inhibiting reuptake. Details and procedural information for this assay are described in Example 9. The x-axis is the log [dose] concentration measured in molar and the y-axis is the [³H]-labeled 5-HT reuptake measured in percent.

FIG. 7B is a graph showing results from an in vitro rat synaptosome serotonin release assay. The graphs display [³H]-labeled 5-HT release as a function of concentration for RS-5-MBPB, R-5-MBPB, and S-5-MBPB. These data indicate that each tested compound rapidly increases extracellular serotonin by stimulating release. Details and procedural information for this assay are described in Example 9. The x-axis is the log [dose] concentration measured in molar and the y-axis is the [³H]-labeled 5-HT release measured in percent.

FIG. 8A is a graph showing results from an in vitro rat synaptosome serotonin uptake inhibition assay. The graphs display percent reuptake of [³H]-labeled 5-HT as a function of concentration for RS-6-MBPB, R-6-MBPB, and S-6-MBPB. This data indicates that each tested compound rapidly increases extracellular serotonin by inhibiting reuptake. Details and procedural information for this assay are described in Example 9. The x-axis is the log [dose] concentration measured in molar and the y-axis is the [³H]-labeled 5-HT reuptake measured in percent.

FIG. 8B is a graph showing results from an in vitro rat synaptosome serotonin release assay. The graphs display [³H]-labeled 5-HT release as a function of concentration for RS-6-MBPB, R-6-MBPB, and S-6-MBPB. These data indicate that each tested compound rapidly increases extracellular serotonin by stimulating release. Details and procedural information for this assay are described in Example 9. The x-axis is the log [dose] concentration measured in molar and the y-axis is the [³H]-labeled 5-HT release measured in percent.

FIG. 8C is a graph showing results from in vitro rat synaptosome dopamine and norepinephrine release assays. The graphs display estimated [³H]-labeled dopamine and norepinephrine release as a function of concentration for S-5-MBPB, R-5-MBPB, S-6-MBPB, and R-6-MBPB. Previously presented serotonin results are included for comparison. These data indicate that each tested compound rapidly increases extracellular norepinephrine by stimulating release, but that the R-enantiomers of 5-MBPB and 6-MBPB are dopamine uptake inhibitors. Details and procedural information for this assay are described in Example 9. The x-axis is the log [dose] concentration measured in molar and the y-axis is the [³H]-labeled 5-HT release measured in percent.

FIG. 9A is a graph showing results from an in vitro rat synaptosome serotonin uptake inhibition assay. The graphs display percent reuptake of [³H]-labeled 5-HT as a function of concentration for R-5-MAPB and S-5-MAPB. This data indicates that each tested compound rapidly increases extracellular serotonin by inhibiting reuptake. Details and procedural information for this assay are described in Example 9. The x-axis is the log [dose] concentration measured in molar and the y-axis is the [³H]-labeled 5-HT reuptake measured in percent.

FIG. 9B is a graph showing results from an in vitro rat synaptosome serotonin efflux assay. The graphs display [³H]-labeled 5-HT release as a function of concentration for R-5-MAPB and S-5-MAPB. These data indicate that each tested compound rapidly increases extracellular serotonin by stimulating release. Details and procedural information for this assay are described in Example 9. The x-axis is the log [dose] concentration measured in molar and the y-axis is the [³H]-labeled 5-HT release measured in percent.

FIG. 10A is a graph showing results from an in vitro rat synaptosome serotonin uptake inhibition assay. The graphs display percent reuptake of [³H]-labeled 5-HT as a function of concentration for R-6-MAPB and S-6-MAPB. This data indicates that each tested compound rapidly increases extracellular serotonin by inhibiting reuptake. Details and procedural information for this assay are described in Example 9. The x-axis is the log [dose] concentration measured in molar and the y-axis is the [³H]-labeled 5-HT reuptake measured in percent.

FIG. 10B is a graph showing results from an in vitro rat synaptosome serotonin efflux assay. The graphs display [³H]-labeled 5-HT release as a function of concentration for R-6-MAPB and S-6-MAPB. These data indicate that each tested compound rapidly increases extracellular serotonin by stimulating release. Details and procedural information for this assay are described in Example 9. The x-axis is the log [dose] concentration measured in molar and the y-axis is the [³H]-labeled 5-HT release measured in percent.

FIG. 11A is a graph showing results from an in vitro rat synaptosome serotonin uptake inhibition assay. The graphs display percent reuptake of [³H]-labeled 5-HT as a function of concentration for (−)-Bk-5-MAPB and (+)-Bk-5-MAPB. This data indicates that each tested compound rapidly increases extracellular serotonin by inhibiting reuptake. Details and procedural information for this assay are described in Example 9. The x-axis is the log [dose] concentration measured in molar and the y-axis is the [³H]-labeled 5-HT reuptake measured in percent.

FIG. 11B is a graph showing results from an in vitro rat synaptosome serotonin efflux assay. The graphs display [³H]-labeled 5-HT release as a function of concentration for (−)-Bk-5-MAPB and (+)-Bk-5-MAPB. These data indicate that each tested compound rapidly increases extracellular serotonin by stimulating release. Details and procedural information for this assay are described in Example 9. The x-axis is the log [dose] concentration measured in molar and the y-axis is the [³H]-labeled 5-HT release measured in percent.

FIG. 12A is a graph showing results from an in vitro rat synaptosome serotonin uptake inhibition assay. The graphs display percent reuptake of [³H]-labeled 5-HT as a function of concentration for (−)-Bk-6-MAPB and (+)-Bk-6-MAPB. This data indicates that each tested compound rapidly increases extracellular serotonin by inhibiting reuptake. Details and procedural information for this assay are described in Example 9. The x-axis is the log [dose] concentration measured in molar and the y-axis is the [³H]-labeled 5-HT reuptake measured in percent.

FIG. 12B is a graph showing results from an in vitro rat synaptosome serotonin efflux assay. The graphs display [³H]-labeled 5-HT release as a function of concentration for (−)-Bk-6-MAPB and (+)-Bk-6-MAPB. These data indicate that each tested compound rapidly increases extracellular serotonin by stimulating release. Details and procedural information for this assay are described in Example 9. The x-axis is the log [dose] concentration measured in molar and the y-axis is the [³H]-labeled 5-HT release measured in percent.

FIG. 13 is a PXRD Diffractogram of 5-MAPB HCl Pattern 1A (5-MAPB hydrochloride or 5-MAPB HCl). The diffractogram confirms the crystalline nature of Pattern 1, The x axis measures 2Theta in degrees and the y axis measures intensity measured in arb. units. The XRPD was taken using the procedure described in Example 12.

FIG. 14 is a PXRD Diffractogram of 5-MAPB Freebase recovered following Liquid-Liquid Extraction. The XRPD diffractogram showed that 5-MAPB Freebase was obtained as described in Example 11 and shown in Table 7. The diffractogram confirms the amorphous nature of 5-MAPB Freebase. The x axis measures 2Theta in degrees and the y axis measures intensity measured in arb. units.

FIG. 15 is a comparison of XRPD Diffractogram salt screening of 5-MAPB HCl Pattern 1A (5-MAPB HCl), Pattern 2A (5-MAPB HBr) and Pattern 4A (5-MAPB H₃PO₄) in various solvents. The diffractogram confirms the crystalline nature of 5-MAPB in various counterions of 5-MAPB HCl Pattern 1A (5-MAPB HCl), 5-MAPB HCl Pattern 1A (5-MAPB HCl in acetone), 5-MAPB HCl Pattern 1A (5-MAPB HCl in MeOH:H₂O 90:10), Pattern 2A (5-MAPB HBr in MeOH:H₂O 90:10) and Pattern 4A (5-MAPB H₃PO₄ in acetone). The salt screening conditions are provided in Example 13 and shown in Table 9. The x axis measures 2Theta in degrees and the y axis measures intensity measured in arb. units.

FIG. 16 is a comparison of XRPD Diffractogram of Pattern 9A (5-MAPB oxalic acid) and Pattern 10A (5-MAPB maleic acid) in various solvents, and solvents oxalic acid and maleic acid. The diffractogram confirms the crystalline nature of 5-MAPB of Pattern 9A (5-MAPB oxalic acid in acetone), Pattern 9A (5-MAPB oxalic acid in MeOH:H₂O 90:10), Pattern 10A (5-MAPB maleic acid in acetone), and Pattern 10A (5-MAPB maleic acid in MeOH:H₂O 90:10). The salt screening conditions are provided in in Example 13 and shown in Table 9. The x axis measures 2Theta in degrees and the y axis measures intensity measured in arb. units.

FIG. 17 is a comparison of XRPD Diffractogram of 5-MAPB HCl Pattern 1A, Pattern 2A (5-MAPB HBr) and Pattern 4B (5-MAPB H₃PO₄) in various solvents. The diffractograms confirm the crystalline nature of 5-MAPB HCl Pattern 1A (5-MAPB HCl), 5-MAPB HCl Pattern 1A (5-MAPB HCl in DCM), 5-MAPB HCl Pattern 1A (5-MAPB HCl in EtOH:H₂O 90:10), Pattern 2A (5-MAPB HBr in EtOH:H₂O 90:10), Pattern 4B (5-MAPB H₃PO₄ in DCM) and Pattern 4B (5-MAPB H₃PO₄ in EtOH:H₂O 90:10). The salt screening conditions are provided in Example 14 and shown in Table 10. The x axis measures 2Theta in degrees and the y axis measures intensity measured in arb. units.

FIG. 18 is a comparison of XRPD Diffractogram of Pattern 9A (5-MAPB oxalic acid) and Pattern 10A (5-MAPB maleic acid) in various solvents, and solvents oxalic acid and maleic acid. The diffractogram confirms the crystalline nature of Pattern 9A (5-MAPB oxalic acid in DCM), Pattern 9A (5-MAPB oxalic acid in EtOH:H₂O 90:10), Pattern 10A (5-MAPB maleic acid in DCM), and Pattern 10A (5-MAPB maleic acid in EtOH:H₂O 90:10). The salt screening methods are provided in Example 14 and shown in Table 10. The x axis measures 2Theta in degrees and the y axis measures intensity measured in arb. units.

FIG. 19 is a comparison of XRPD Diffractogram of Pattern 4 (5-MAPB H₃PO₄) in various solvents. The diffractogram confirms the crystalline nature of Pattern 4A (5-MAPB H₃PO₄ in acetone), Pattern 4B (5-MAPB H₃PO₄ in DCM) and Pattern 4C (5-MAPB H₃PO₄ in THF). The salt screening conditions are described in Example 15 and shown in Table 11. The x axis measures 2Theta in degrees and the y axis measures intensity measured in arb. units.

FIG. 20 is an optical micrograph of 5-MAPB HCl Pattern 1A. 5-MAPB HCl Pattern 1A appeared to have a morphology of irregular agglomerates.

FIG. 21 is an optical micrograph of 5-MAPB HBr Pattern 2B (scale-up of Pattern 2A). Pattern 2B appeared to have a morphology of irregular agglomerates.

FIG. 22 is an optical micrograph of 5-MAPB Pattern 10A. Pattern 10A appeared to have a morphology of irregular agglomerates.

FIG. 23 is a PXRD Diffractogram of S-5-MAPB HCl Pattern 1A (S-5-MAPB HCl). The diffractogram confirms the crystalline nature of S-5-MAPB HCl Pattern 1A. The x axis measures 2Theta in degrees and the y axis measures intensity measured in arb. units.

FIG. 24 is a comparison of XRPD Diffractogram of S-5-MAPB HCl Pattern 1A (P1AE) formed from various solvents. The diffractogram confirms the crystalline nature of S-5-MAPB HCl Pattern 1A (5-MAPB HCl Pure Enantiomer, P1AE) in various conditions. The XRPD diffractogram shows several salts as described in Example 17 and shown in Table 13. The x axis measures 2Theta in degrees and the y axis measures intensity measured in arb. units.

FIG. 25 is a comparison of XRPD Diffractogram of S-5-MAPB Pattern 2A (5-MAPB Enantiomer HBr) and S-5-MAPB Pattern 4A (5-MAPB Enantiomer H₃PO₄) in various solvents. The diffractogram confirms the crystalline nature of these salts in various conditions. The salt screen methods are provided in Example 17 and shown in Table 13. The x axis measures 2Theta in degrees and the y axis measures intensity measured in arb. units.

FIG. 26 is a comparison of XRPD Diffractogram of oxalic acid and Pattern 8A Enantiomer (Pattern 8A, 5-MAPB Enantiomer oxalic acid) in various solvents. The diffractogram confirms the crystalline nature of S-5-MAPB Pattern 8A (5-MAPB Enantiomer oxalic acid) in various conditions. The salt screen methods are provided in Example 17 and shown in Table 13. The x axis measures 2Theta in degrees and the y axis measures intensity measured in arb. units.

FIG. 27 is a comparison of XRPD Diffractogram of S-5-MAPB HCl Pattern 1A (P1AE) in various solvents. The diffractogram confirms the crystalline nature of S-5-MAPB HCl Pattern 1A under several conditions. The XRPD diffractogram shows several conditions as described in Example 18 and shown in Table 14. The x axis measures 2Theta in degrees and the y axis measures intensity measured in arb. units.

FIG. 28 is a comparison of XRPD Diffractogram of S-5-MAPB HBr Pattern 2A (5-MAPB Enantiomer HBr) and S-5-MAPB Pattern 4A (5-MAPB Enantiomer H₃PO₄) in various solvents. The salt screen methods are provided in Example 18 and shown in Table 14. The x axis measures 2Theta in degrees and the y axis measures intensity measured in arb. units.

FIG. 29 is a comparison of XRPD Diffractogram of oxalic acid and S-5-MAPB Pattern 8A (Pattern 8AE, 5-MAPB Enantiomer oxalic acid) in various solvents. The diffractogram confirms the crystalline nature of Pattern 8A. The salt screen methods are provided in Example 18 and shown in Table 14. The x axis measures 2Theta in degrees and the y axis measures intensity measured in arb. units.

FIG. 30 is a comparison of XRPD Diffractogram of fumaric acid and S-5-MAPB Pattern 10A (Pattern 10AE, 5-MAPB Enantiomer fumaric acid) in EtOH/H₂O 90:10. The diffractogram confirms the crystalline nature of Pattern 10A. The salt screen methods are provided in Table 14. The x axis measures 2Theta in degrees and the y axis measures intensity measured in arb. units.

FIG. 31 is a comparison of XRPD Diffractogram of S-5-MAPB HCl Pattern 1A (Pattern 1AE, 5-MAPB Enantiomer HCl or ACN), Pattern 2A Enantiomer (Pattern 2AE, 5-MAPB Enantiomer HBr) and Pattern 4A Enantiomer (Pattern 4AE, 5-MAPB Enantiomer H₃PO₄). The x axis measures 2Theta in degrees and the y axis measures intensity measured in arb. units.

FIG. 32 is an optical micrograph of S-5-MAPB HCl Pattern 1A. S-5-MAPB HCl Pattern 1A appeared to have an irregular morphology.

FIG. 33 is an optical micrograph of S-5-MAPB Pattern 4A (Pattern 4AE). Pattern 4AE appeared to have a morphology of irregular agglomerates and fine particles.

FIG. 34 is an optical micrograph of S-5-MAPB Pattern 8A (Pattern 8AE). Pattern 8AE appeared to have a morphology of irregular agglomerates.

FIG. 35 is a differential scanning calorimetry (DSC) and thermogravimetric analysis (TGA) of 5-MAPB HCl Pattern 1A (HCl). The DSC shows an endotherm (likely melt) w/onset ˜194° C. and the TGA shows ˜0.09% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 36 is a differential scanning calorimetry (DSC) and thermogravimetric analysis (TGA) of 5-MAPB Pattern 2A (HBr). The DSC shows an endotherm (likely melt) w/onset ˜135° C. and the shows ˜2.00% weight loss up to 150° C. and decomposition at higher temperatures (>240° C.). The methods used for the DSC/TGA was conducted as described in Example 20 Table 16. The x-axis is temperature measured in degrees Celsius and the y-axis is Weight measured in percentage and Heat flow measured in W/g.

FIG. 37 is a differential scanning calorimetry (DSC) and thermogravimetric analysis (TGA) of 5-MAPB Pattern 4A (H₃PO₄). The DSC shows endotherm (likely melt and decomposition) w/onset ˜178° C. and the TGA shows ˜0.01% weight loss up to 150° C. and decomposition at higher temperatures (>180° C.). The methods used for the DSC/TGA was conducted as described in Example 20 Table 16. The x-axis is temperature measured in degrees Celsius and the y-axis is Weight measured in percentage and Heat flow measured in W/g.

FIG. 38 is a differential scanning calorimetry (DSC) and thermogravimetric analysis (TGA) of 5-MAPB Pattern 4B (H₃PO₄). The DSC shows no clear thermal events and the TGA shows ˜0.42% weight loss up to 150° C. The methods used for the DSC/TGA was conducted as described in Example 20 Table 16. The x-axis is temperature measured in degrees Celsius and the y-axis is Weight measured in percentage and Heat flow measured in W/g.

FIG. 39 is a differential scanning calorimetry (DSC) and thermogravimetric analysis (TGA) of 5-MAPB Pattern 4C (H₃PO₄). The DSC shows a broad endotherm w/onset at ˜133° C. and the TGA shows ˜2.82% weight loss up to 140° C. The methods used for the DSC/TGA was conducted as described in Example 20 Table 16. The x-axis is temperature measured in degrees Celsius and the y-axis is Weight measured in percentage and Heat flow measured in W/g.

FIG. 40 is a differential scanning calorimetry (DSC) and thermogravimetric analysis (TGA) of 5-MAPB Pattern 9A (Oxalic). The DSC shows an endotherm w/onset at ˜122° C. and the TGA shows ˜1.37% weight loss up to 150° C. and decomposition at higher temperatures (>180° C.). The methods used for the DSC/TGA was conducted as described in Example 20 Table 16. The x-axis is temperature measured in degrees Celsius and the y-axis is Weight measured in percentage and Heat flow measured in W/g.

FIG. 41 is a differential scanning calorimetry (DSC) and thermogravimetric analysis (TGA) of 5-MAPB Pattern 10A (Maleic). The DSC shows an endotherm w/onset at ˜117° C. and the TGA shows ˜0.45% weight loss up to 150° C. and decomposition at higher temperatures (>160° C.). The methods used for the DSC/TGA was conducted as described in Example 20 Table 16. The x-axis is temperature measured in degrees Celsius and the y-axis is Weight measured in percentage and Heat flow measured in W/g.

FIG. 42 is a differential scanning calorimetry (DSC) and thermogravimetric analysis (TGA) of S-5-MAPB HCl Pattern 1A. The DSC shows a sharp endotherm (likely melt) w/onset ˜ 199° C. and the TGA shows ˜0.08% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 43 is a differential scanning calorimetry (DSC) and thermogravimetric analysis (TGA) of S-5-MAPB Pattern 2A (HBr. The DSC shows a sharp endotherm (likely melt) w/onset ˜161° C. and the TGA shows ˜1.68% weight loss up to 160° C. The methods used for the DSC/TGA was conducted as described in Example 20 Table 16. The x-axis is temperature measured in degrees Celsius and the y-axis is Weight measured in percentage and Heat flow measured in W/g.

FIG. 44 is a differential scanning calorimetry (DSC) and thermogravimetric analysis (TGA) of S-5-MAPB Pattern 4A (H₃PO₄. The DSC shows no clear thermal events and a noisy baseline at higher temps (>150° C.) and the TGA shows ˜0.55% weight loss up to 150° C. and decomposition at higher temperatures (>180° C.). The methods used for the DSC/TGA was conducted as described in Example 20 Table 16. The x-axis is temperature measured in degrees Celsius and the y-axis is Weight measured in percentage and Heat flow measured in W/g.

FIG. 45 is a differential scanning calorimetry (DSC) and thermogravimetric analysis (TGA) of S-5-MAPB Pattern 8A (Oxalic). The DSC shows an endotherm w/onset at ˜146° C. and the TGA (blue curve) shows ˜0.58% weight loss up to 150° C. The methods used for the DSC/TGA was conducted as described in Example 20 Table 16. The x-axis is temperature measured in degrees Celsius and the y-axis is Weight measured in percentage and Heat flow measured in W/g.

FIG. 46 is a differential scanning calorimetry (DSC) and thermogravimetric analysis (TGA) of S-5-MAPB Pattern 10A (Fumaric). The DSC shows a broad endotherm w/peaks at ˜106° C. and ˜124° C. and the TGA shows ˜0.62% weight loss up to 140° C. and decomposition at higher temperatures (>180° C.). The methods used for the DSC/TGA was conducted as described in Example 20 Table 16. The x-axis is temperature measured in degrees Celsius and the y-axis is Weight measured in percentage and Heat flow measured in W/g.

FIG. 47 is a PXRD Diffractogram of R-5-MAPB HCl used in the Liquid-Liquid Extraction to afford R-5-MAPB as described in Example 25. The x axis measures 2Theta in degrees and the y axis measures intensity measured in arb. units.

FIG. 48 provides the names and structures of select entactogenic compounds referred to herein.

FIG. 49 is a PXRD Diffractogram of S-6-MAPB HCl Pattern 1A. The diffractogram confirms the crystalline nature of Pattern 1A. The x axis is 2Theta measured in degrees and the y axis is intensity measured in arbitrary units. The XRPD was taken using the procedure described in Example 12.

FIG. 50 is a comparison of XRPD Diffractograms for S-6-MAPB HCl Pattern 1A and S-6-MAPB HBr Pattern 2A prepared from multiple conditions. The salt screening conditions are provided in Example 31 and shown in Table 25. The x axis is 2Theta measured in degrees and the y axis is intensity measured in arbitrary units. The XRPD was taken using the procedure described in Example 12.

FIG. 51 is a comparison of XRPD Diffractograms for S-6-MAPB HCl Pattern 1A, oxalic acid, and S-6-MAPB oxalate Pattern 5A prepared from multiple conditions. The salt screening conditions are provided in Example 31 and shown in Table 25. The x axis is 2Theta measured in degrees and the y axis is intensity measured in arbitrary units. The XRPD was taken using the procedure described in Example 12.

FIG. 52 is a comparison of XRPD Diffractograms for S-6-MAPB HCl Pattern 1A, S-6-MAPB H₃PO₄ Pattern 3A and S-6-MAPB H₃PO₄ Pattern 3B. The salt screening conditions are provided in Example 31 and shown in Table 25. The x axis is 2Theta measured in degrees and the y axis is intensity measured in arbitrary units. The XRPD was taken using the procedure described in Example 12.

FIG. 53 is a comparison of XRPD Diffractograms for S-6-MAPB HCl Pattern 1A and S-6-MAPB HBr Pattern 2A prepared from several different conditions. The salt screening conditions are provided in Example 32 and shown in Table 26. The x axis is 2Theta measured in degrees and the y axis is intensity measured in arbitrary units. The XRPD was taken using the procedure described in Example 12.

FIG. 54 is a comparison of XRPD Diffractograms for S-6-MAPB HCl Pattern 1A, oxalic acid, S-6-MAPB oxalate Pattern 5A, and S-6-MAPB H₃PO₄ Pattern 3A prepared from several different conditions. The salt screening conditions are provided in Example 32 and shown in Table 26. The x axis is 2Theta measured in degrees and the y axis is intensity measured in arbitrary units. The XRPD was taken using the procedure described in Example 12.

FIG. 55 is a comparison of XRPD Diffractograms for S-6-MAPB HCl Pattern 1A, S-6-MAPB HBr Pattern 2A, and S-6-MAPB H₃PO₄ Pattern 3A. The salt screening conditions are provided in Example 33 and shown in Table 27. The x axis is 2Theta measured in degrees and the y axis is intensity measured in arbitrary units. The XRPD was taken using the procedure described in Example 12.

FIG. 56 is a comparison of XRPD Diffractograms for S-6-MAPB HCl Pattern 1A, oxalic acid, and S-6-MAPB oxalate Pattern 5A. The salt screening conditions are provided in Example 33 and shown in Table 27. The x axis is 2Theta measured in degrees and the y axis is intensity measured in arbitrary units. The XRPD was taken using the procedure described in Example 12.

FIG. 57 is a differential scanning calorimetry (DSC) and thermogravimetric analysis (TGA) of S-6-MAPB HCl Pattern 1A. The DSC shows an endotherm with an onset of about 199° C. and the TGA shows about 0.12% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 58 is a differential scanning calorimetry (DSC) and thermogravimetric analysis (TGA) of S-6-MAPB HBr Pattern 2A. The DSC shows two endotherms with an onset of about 70° C. and the other at about 186° C. and the TGA shows about 0.17% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 59 is a differential scanning calorimetry (DSC) and thermogravimetric analysis (TGA) of S-6-MAPB H₃PO₄ Pattern 3A. The DSC shows two endotherms with an onset of about 90° C. and the other at about 179° C. and the TGA shows about 0.27% weight loss up to 150° C. and decomposition at higher temperatures (>180° C.). The methods used for the DSC/TGA was conducted as described in Example 20 Table 16. The x-axis is temperature measured in degrees Celsius and the y-axis is Weight measured in percentage and Heat flow measured in W/g.

FIG. 60 is a differential scanning calorimetry (DSC) and thermogravimetric analysis (TGA) of S-6-MAPB H₃PO₄ Pattern 3B. The DSC shows a sharp endotherm with an onset of about 188° C. and the TGA shows about 0.14% weight loss up to 150° C. and decomposition at higher temperatures (>180° C.). The methods used for the DSC/TGA was conducted as described in Example 20 Table 16. The x-axis is temperature measured in degrees Celsius and the y-axis is Weight measured in percentage and Heat flow measured in W/g.

FIG. 61 is a differential scanning calorimetry (DSC) and thermogravimetric analysis (TGA) of S-6-MAPB oxalate Pattern 5A. The DSC shows two endotherms one with an onset of about 105° C. and the other with an onset of about 138° C. The TGA shows about 0.29% weight loss up to 150° C. and decomposition at higher temperatures (>180° C.). The methods used for the DSC/TGA was conducted as described in Example 20 Table 16. The x-axis is temperature measured in degrees Celsius and the y-axis is Weight measured in percentage and Heat flow measured in W/g.

FIG. 62 is a XRPD Diffractogram of S-BK-5-MAPB Pattern 1A (Pattern 1AE, S-BK-5-MAPB Enantiomer HCl) in acetone. The diffractogram confirms the crystalline nature of Pattern 1A. The liquid-liquid extraction method used to isolate the enantiomer is provided in Example 34. The x axis measures 2Theta in degrees and the y axis measures intensity measured in arb. units.

FIG. 63 is a XRPD Diffractogram of S-BK-5-MAPB Pattern 1B (Pattern 1BE, S-BK-5-MAPB Enantiomer HCl) in acetone. The diffractogram confirms the crystalline nature of Pattern 1B. The salt screen methods are provided in Example 36 and shown in Table 31. The x axis measures 2Theta in degrees and the y axis measures intensity measured in arb. units.

FIG. 64 is a XRPD Diffractogram of S-BK-5-MAPB Pattern 3A (Pattern 3AE, S-BK-5-MAPB Enantiomer H₂SO₄) in ACN. The diffractogram confirms the crystalline nature of Pattern 3A. The salt screen methods are provided in Example 38 and shown in Table 33. The x axis measures 2Theta in degrees and the y axis measures intensity measured in arb. units.

FIG. 65 is a comparison of XRPD Diffractogram of R-6-MBPB (oxalate salt) and S-6-MBPB (oxalate salt) Pattern 9A in ACN. The diffractogram confirms the crystalline nature of Pattern 9A. The salt screen methods are provided in Examples 44, and 54 and shown in Tables 40, and 50. The x axis measures 2Theta in degrees and the y axis measures intensity measured in arb. units.

FIG. 66 is a XRPD Diffractogram of S-BK-5-MAPB Pattern 10A (Pattern 10AE, S-BK-5-MAPB Enantiomer maleic) in acetone. The diffractogram confirms the crystalline nature of Pattern 10A. The salt screen methods are provided in Example 36 and shown in Table 31. The x axis measures 2Theta in degrees and the y axis measures intensity measured in arb. units.

FIG. 67 is a XRPD Diffractogram of S-BK-5-MAPB Pattern 11A (Pattern 11AE, S-BK-5-MAPB Enantiomer malic) in MeOH:water (9:1). The diffractogram confirms the crystalline nature of Pattern 11A. The salt screen methods are provided in Example 37 and shown in Table 32. The x axis measures 2Theta in degrees and the y axis measures intensity measured in arb. units.

FIG. 68 is a XRPD Diffractogram of S-BK-5-MAPB Pattern 13A (Pattern 13AE, S-BK-5-MAPB Enantiomer fumaric) in acetone. The diffractogram confirms the crystalline nature of Pattern 13A. The salt screen methods are provided in Example 36 and shown in Table 31. The x axis measures 2Theta in degrees and the y axis measures intensity measured in arb. units.

FIG. 69 is a XRPD Diffractogram of S-BK-5-MAPB Pattern 14A (Pattern 14AE, S-BK-5-MAPB Enantiomer benzoic) in ACN. The diffractogram confirms the crystalline nature of Pattern 14A. The salt screen methods are provided in Example 38 and shown in Table 33. The x axis measures 2Theta in degrees and the y axis measures intensity measured in arb. units.

FIG. 70 is a XRPD Diffractogram of S-BK-5-MAPB Pattern 15A (Pattern 15AE, S-BK-5-MAPB Enantiomer salicylic) in acetone. The diffractogram confirms the crystalline nature of Pattern 15A. The salt screen methods are provided in Example 36 and shown in Table 31. The x axis measures 2Theta in degrees and the y axis measures intensity measured in arb. units.

FIG. 71 is a XRPD Diffractogram of S-BK-5-MAPB Pattern 15B (Pattern 15BE, S-BK-5-MAPB Enantiomer salicylic) in ACN. The diffractogram confirms the crystalline nature of Pattern 15B. The salt screen methods are provided in Example 38 and shown in Table 33. The x axis measures 2Theta in degrees and the y axis measures intensity measured in arb. units.

FIG. 72 is a comparison of XRPD Diffractogram of S-BK-5-MAPB Pattern 1A (Pattern 1AE, S-BK-5-MAPB Enantiomer HCl) in various solvents. The diffractogram confirms the crystalline nature of Pattern 1A. The salt screen methods are provided in Examples 36, and 37 and shown in Tables 31, and 32. The x axis measures 2Theta in degrees and the y axis measures intensity measured in arb. units.

FIG. 73 is a comparison of XRPD Diffractogram of S-BK-5-MAPB Pattern 10A (Pattern 10AE, S-BK-5-MAPB Enantiomer maleic) in various solvents. The diffractogram confirms the crystalline nature of Pattern 10A. The salt screen methods are provided in Examples 36, and 37 and shown in Tables 31, and 32. The x axis measures 2Theta in degrees and the y axis measures intensity measured in arb. units.

FIG. 74 is a comparison of XRPD Diffractogram of S-BK-5-MAPB salts (HBr, H₂SO₄, and H₃PO₄) in various solvents. The diffractogram confirms the crystalline nature of the Patterns. The salt screen methods are provided in Examples 36, 37, and 38 and shown in Tables 31, 32, and 33. The x axis measures 2Theta in degrees and the y axis measures intensity measured in arb. units.

FIG. 75 is a comparison of XRPD Diffractogram of S-BK-5-MAPB salts (HNO₃, methanesulfonic, and citric) in various solvents. The diffractogram confirms the crystalline nature of the Patterns. The salt screen methods are provided in Examples 36, 37, and 38 and shown in Tables 31, 32, and 33. The x axis measures 2Theta in degrees and the y axis measures intensity measured in arb. units.

FIG. 76 is a comparison of XRPD Diffractogram of S-BK-5-MAPB MAPB Enantiomer oxalate Pattern 9A in various solvents. The diffractogram confirms the crystalline nature of the Pattern 9A. The salt screen methods are provided in Examples 37, and 38 and shown in Tables 32, and 33. The x axis measures 2Theta in degrees and the y axis measures intensity measured in arb. units.

FIG. 77 is a comparison of XRPD Diffractogram of S-BK-5-MAPB Pattern 11A (Pattern 11AE, S-BK-5-MAPB Enantiomer malic) in MeOH:water (9:1). The diffractogram confirms the crystalline nature of Pattern 11A. The salt screen methods are provided in Example 37 and shown in Table 32. The x axis measures 2Theta in degrees and the y axis measures intensity measured in arb. units.

FIG. 78 is a comparison of XRPD Diffractogram of S-BK-5-MAPB Pattern 13A (Pattern 13AE, S-BK-5-MAPB Enantiomer fumaric) in various solvents. The diffractogram confirms the crystalline nature of Pattern 13A. The salt screen methods are provided in Example 36, and 38 and shown in Tables 31, and 33. The x axis measures 2Theta in degrees and the y axis measures intensity measured in arb. units.

FIG. 79 is a comparison of XRPD Diffractogram of S-BK-5-MAPB Pattern 15B (Pattern 15BE, S-BK-5-MAPB Enantiomer salicylic) in acetone. The diffractogram confirms the crystalline nature of Pattern 15B. The salt screen methods are provided in Example 36 and shown in Table 31. The x axis measures 2Theta in degrees and the y axis measures intensity measured in arb. units.

FIG. 80 is a comparison of XRPD Diffractogram of S-BK-5-MAPB Pattern 1A (Pattern 1AE, S-BK-5-MAPB Enantiomer HCl) in various solvents. The diffractogram confirms the crystalline nature of Pattern 1A. The salt screen methods are provided in Examples 36, 38, and 39 and shown in Tables 31, 33 and 34. The x axis measures 2Theta in degrees and the y axis measures intensity measured in arb. units.

FIG. 81 is a comparison of XRPD Diffractogram of S-BK-5-MAPB Pattern 3A and vacuum dried sample (Pattern 3AE, S-BK-5-MAPB Enantiomer H₂SO₄) in ACN. The diffractogram confirms the crystalline nature of Pattern 3A. The salt screen methods are provided in Example 38 and shown in Table 33. The x axis measures 2Theta in degrees and the y axis measures intensity measured in arb. units.

FIG. 82 is a comparison of XRPD Diffractogram of S-BK-5-MAPB salts (H₃PO₄, HNO₃, and tartaric) in ACN. The diffractogram confirms the crystalline nature of the Patterns. The salt screen methods are provided in Example 38 and shown in Table 33. The x axis measures 2Theta in degrees and the y axis measures intensity measured in arb. units.

FIG. 83 is a comparison of XRPD Diffractogram of S-BK-5-MAPB Pattern 9A (Pattern 9AE, S-BK-5-MAPB Enantiomer oxalate) in various solvents. The diffractogram confirms the crystalline nature of Pattern 9A. The salt screen methods are provided in Examples 36, and 38 and shown in Tables 31, and 33. The x axis measures 2Theta in degrees and the y axis measures intensity measured in arb. units.

FIG. 84 is a comparison of XRPD Diffractogram of S-BK-5-MAPB Pattern 10A (Pattern 10AE, S-BK-5-MAPB Enantiomer maleic salt) in various solvents. The diffractogram confirms the crystalline nature of Pattern 10A. The salt screen methods are provided in Examples 36, and 38 and shown in Tables 31, and 33. The x axis measures 2Theta in degrees and the y axis measures intensity measured in arb. units.

FIG. 85 is a comparison of XRPD Diffractogram of S-BK-5-MAPB Enantiomer citric salt in ACN. The diffractogram confirms the crystalline nature of the Pattern. The salt screen methods are provided in Example 38 and shown in Table 33. The x axis measures 2Theta in degrees and the y axis measures intensity measured in arb. units.

FIG. 86 is a comparison of XRPD Diffractogram of S-BK-5-MAPB Pattern 13A (Pattern 13AE, S-BK-5-MAPB Enantiomer fumaric salt) in various solvents. The diffractogram confirms the crystalline nature of Pattern 13A. The salt screen methods are provided in Examples 36, 37, and 38 and shown in Tables 31, 32, and 33. The x axis measures 2Theta in degrees and the y axis measures intensity measured in arb. units.

FIG. 87 is a comparison of XRPD Diffractogram of S-BK-5-MAPB Pattern 14A (Pattern 14AE, S-BK-5-MAPB Enantiomer benzoic salt) in ACN. The diffractogram confirms the crystalline nature of Pattern 14A. The salt screen methods are provided in Example 38 and shown in Table 33. The x axis measures 2Theta in degrees and the y axis measures intensity measured in arb. units.

FIG. 88 is a comparison of XRPD Diffractogram of S-BK-5-MAPB Pattern 15A (Pattern 15AE, S-BK-5-MAPB Enantiomer salicylic salt) in acetone and S-BK-5-MAPB Pattern 15B (Pattern 15BE, S-BK-5-MAPB Enantiomer salicylic salt) in ACN. The diffractogram confirms the crystalline nature of Patterns 15A and 15B. The salt screen methods are provided in Examples 36, and 38 and shown in Tables 31, and 33. The x axis measures 2Theta in degrees and the y axis measures intensity measured in arb. units.

FIG. 89 is a differential scanning calorimetry (DSC) and thermogravimetric analysis (TGA) of S-BK-5-MAPB Pattern 1B (HCl). The DSC shows a sharp endotherm (likely melt) w/onset at ˜196° C. and the TGA shows ˜1.80% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 90 is a differential scanning calorimetry (DSC) and thermogravimetric analysis (TGA) of S-BK-5-MAPB Pattern 3A (H₂SO₄). The DSC shows a large endotherm (likely melt) w/onset at ˜61° C. and the TGA shows ˜2.34% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 91 is a differential scanning calorimetry (DSC) and thermogravimetric analysis (TGA) of S-BK-5-MAPB Pattern 9A (oxalate). The DSC shows a small endotherm (likely melt) w/onset at ˜51° C. and the TGA shows ˜4.53% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 92 is a differential scanning calorimetry (DSC) and thermogravimetric analysis (TGA) of S-BK-5-MAPB Pattern 10A (maleic). The DSC shows a sharp endotherm (likely melt) w/onset at ˜134° C. and the TGA shows ˜3.91% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 93 is a differential scanning calorimetry (DSC) and thermogravimetric analysis (TGA) of S-BK-5-MAPB Pattern 11A (malic). The DSC shows a broad split endotherm with peaks at ˜116° C. and 125° C., and the TGA shows ˜4.26% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 94 is a differential scanning calorimetry (DSC) and thermogravimetric analysis (TGA) of S-BK-5-MAPB Pattern 13A (fumaric). The DSC shows a broad endotherm (likely melt) w/onset at ˜76° C., a large split endotherm (likely melt) with peaks at 133° C. and 155° C., and the TGA shows ˜1.03% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 95 is a differential scanning calorimetry (DSC) and thermogravimetric analysis (TGA) of S-BK-5-MAPB Pattern 14A (benzoic). The DSC shows a large, broad endotherm (likely melt and decomposition) with onset at ˜123° C., and the TGA shows no significant weight loss up to 100° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 96 is a differential scanning calorimetry (DSC) and thermogravimetric analysis (TGA) of S-BK-5-MAPB Pattern 15A (salicylic). The DSC shows a large endotherm with onset at ˜71° C., a small endotherm with onset at 120° C., and the TGA shows ˜8.27% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 97 is a differential scanning calorimetry (DSC) and thermogravimetric analysis (TGA) of S-BK-5-MAPB Pattern 15B (salicylic). The DSC shows a small, broad endotherm (likely melt) with onset at ˜40° C., and the TGA shows ˜0.37% weight loss up to 100° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 98 is a XRPD Diffractogram of S-6-MBPB Pattern 1A (Pattern 1AE, S-6-MBPB Enantiomer HCl) in acetone. The diffractogram confirms the crystalline nature of Pattern 1A. The liquid-liquid extraction method used to isolate the enantiomer is provided in Examples 40, and 42 and Table 38. The x axis measures 2Theta in degrees and the y axis measures intensity measured in arb. units.

FIG. 99 is a differential scanning calorimetry (DSC) and thermogravimetric analysis (TGA) of S-6-MBPB Pattern 15A (salicylic). The DSC shows a small broad endotherm with onset at ˜124° C., and a sharp endotherm (likely melt) with onset at ˜168° C., and the TGA shows ˜2.26% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 100 is a XRPD Diffractogram of S-6-MBPB Pattern 2A (Pattern 2AE, S-6-MBPB Enantiomer HBr) in ACN. The diffractogram confirms the crystalline nature of Pattern 2A. The salt screen method is provided in Examples 44 and Table 40. The x axis measures 2Theta in degrees and the y axis measures intensity measured in arb. units.

FIG. 101 is a XRPD Diffractogram of S-6-MBPB Pattern 4A (Pattern 4AE, S-6-MBPB Enantiomer H₃PO₄) in ACN. The diffractogram confirms the crystalline nature of Pattern 4A. The salt screen method is provided in Examples 44 and Table 40. The x axis measures 2Theta in degrees and the y axis measures intensity measured in arb. units.

FIG. 102 is a XRPD Diffractogram of S-6-MBPB Pattern 5A (Pattern 5AE, S-6-MBPB Enantiomer HNO₃) in acetone. The diffractogram confirms the crystalline nature of Pattern 5A. The salt screen method is provided in Examples 42 and Table 38. The x axis measures 2Theta in degrees and the y axis measures intensity measured in arb. units.

FIG. 103 is a XRPD Diffractogram of S-6-MBPB Pattern 7A (Pattern 7AE, S-6-MBPB Enantiomer tartaric) in acetone. The diffractogram confirms the crystalline nature of Pattern 7A. The salt screen method is provided in Examples 42 and Table 38. The x axis measures 2Theta in degrees and the y axis measures intensity measured in arb. units.

FIG. 104 is a XRPD Diffractogram of S-6-MBPB Pattern 8A (Pattern 8AE, S-6-MBPB Enantiomer succinic) in acetone. The diffractogram confirms the crystalline nature of Pattern 8A. The salt screen method is provided in Examples 42 and Table 38. The x axis measures 2Theta in degrees and the y axis measures intensity measured in arb. units.

FIG. 105 is a XRPD Diffractogram of S-6-MBPB Pattern 9A (Pattern 9AE, S-6-MBPB Enantiomer oxalate) in acetone. The diffractogram confirms the crystalline nature of Pattern 9A. The salt screen method is provided in Examples 42 and Table 38. The x axis measures 2Theta in degrees and the y axis measures intensity measured in arb. units.

FIG. 106 is a XRPD Diffractogram of S-6-MBPB Pattern 10A (Pattern 10AE, S-6-MBPB Enantiomer maleic) in ACN. The diffractogram confirms the crystalline nature of Pattern 10A. The salt screen method is provided in Examples 44 and Table 40. The x axis measures 2Theta in degrees and the y axis measures intensity measured in arb. units.

FIG. 107 is a XRPD Diffractogram of S-6-MBPB Pattern 12A (Pattern 12AE, S-6-MBPB Enantiomer citric) in ACN. The diffractogram confirms the crystalline nature of Pattern 12A. The salt screen method is provided in Examples 44 and Table 40. The x axis measures 2Theta in degrees and the y axis measures intensity measured in arb. units.

FIG. 108 is a XRPD Diffractogram of S-6-MBPB Pattern 13A (Pattern 13AE, S-6-MBPB Enantiomer fumaric) in MeOH:water (9:1). The diffractogram confirms the crystalline nature of Pattern 13A. The salt screen method is provided in Examples 43 and Table 39. The x axis measures 2Theta in degrees and the y axis measures intensity measured in arb. units.

FIG. 109 is a XRPD Diffractogram of S-6-MBPB Pattern 13B (Pattern 13BE, S-6-MBPB Enantiomer fumaric) in ACN. The diffractogram confirms the crystalline nature of Pattern 13B. The salt screen method is provided in Examples 44 and Table 40. The x axis measures 2Theta in degrees and the y axis measures intensity measured in arb. units.

FIG. 110 is a comparison of XRPD Diffractogram of S-6-MBPB (HCl salt) Pattern 1A in various solvents. The diffractogram confirms the crystalline nature of Pattern 1A. The salt screen methods are provided in Examples 42, and 43 and shown in Tables 38, and 39. The x axis measures 2Theta in degrees and the y axis measures intensity measured in arb. units.

FIG. 111 is a comparison of XRPD Diffractogram of S-6-MBPB (HBr salt) Pattern 2A in various solvents. The diffractogram confirms the crystalline nature of Pattern 2A. The salt screen methods are provided in Examples 42, and 43 and shown in Tables 38, and 39. The x axis measures 2Theta in degrees and the y axis measures intensity measured in arb. units.

FIG. 112 is a comparison of XRPD Diffractogram of S-6-MBPB (HNO₃ salt) Pattern 5A in various solvents. The diffractogram confirms the crystalline nature of Pattern 5A. The salt screen methods are provided in Examples 42, and 43 and shown in Tables 38, and 39. The x axis measures 2Theta in degrees and the y axis measures intensity measured in arb. units.

FIG. 113 is a comparison of XRPD Diffractogram of S-6-MBPB (tartaric salt) Pattern 7A in various solvents. The diffractogram confirms the crystalline nature of Pattern 7A. The salt screen methods are provided in Examples 42, and 43 and shown in Tables 38, and 39. The x axis measures 2Theta in degrees and the y axis measures intensity measured in arb. units.

FIG. 114 is a comparison of XRPD Diffractogram of S-6-MBPB (succinic salt) Pattern 8A in acetone. The diffractogram confirms the crystalline nature of Pattern 8A. The salt screen methods are provided in Example 42 and shown in Table 38. The x axis measures 2Theta in degrees and the y axis measures intensity measured in arb. units.

FIG. 115 is a comparison of XRPD Diffractogram of S-6-MBPB (oxalate salt) Pattern 9A in various solvents. The diffractogram confirms the crystalline nature of Pattern 9A. The salt screen methods are provided in Examples 42, and 43 and shown in Tables 38, and 39. The x axis measures 2Theta in degrees and the y axis measures intensity measured in arb. units.

FIG. 116 is a comparison of XRPD Diffractogram of S-6-MBPB (maleic salt) Pattern 10A in MeOH:water (9:1). The diffractogram confirms the crystalline nature of Pattern 10A. The salt screen methods are provided in Example 43 and shown in Table 39. The x axis measures 2Theta in degrees and the y axis measures intensity measured in arb. units.

FIG. 117 is a comparison of XRPD Diffractogram of S-6-MBPB (citric salt) Pattern 12A in MeOH:water (9:1). The diffractogram confirms the crystalline nature of Pattern 12A. The salt screen methods are provided in Example 43 and shown in Table 39. The x axis measures 2Theta in degrees and the y axis measures intensity measured in arb. units.

FIG. 118 is a comparison of XRPD Diffractogram of S-6-MBPB (fumaric salt) Pattern 13A in various solvents. The diffractogram confirms the crystalline nature of Pattern 13A. The salt screen methods are provided in Examples 42, and 43 and shown in Tables 38, and 39. The x axis measures 2Theta in degrees and the y axis measures intensity measured in arb. units.

FIG. 119 is a comparison of XRPD Diffractogram of S-6-MBPB (HCl salt) Pattern 1A in various solvents. The diffractogram confirms the crystalline nature of Pattern 1A. The salt screen methods are provided in Examples 44, and 45 and shown in Tables 40, and 41. The x axis measures 2Theta in degrees and the y axis measures intensity measured in arb. units.

FIG. 120 is a comparison of XRPD Diffractogram of S-6-MBPB (HBr salt) Pattern 2A in various solvents. The diffractogram confirms the crystalline nature of Pattern 2A. The salt screen methods are provided in Examples 42, and 44 and shown in Tables 38, and 40. The x axis measures 2Theta in degrees and the y axis measures intensity measured in arb. units.

FIG. 121 is a comparison of XRPD Diffractogram of S-6-MBPB (H₃PO₄ salt) Pattern 4A in ACN The diffractogram confirms the crystalline nature of Pattern 4A. The salt screen methods are provided in Example 44 and shown in Table 40. The x axis measures 2Theta in degrees and the y axis measures intensity measured in arb. units.

FIG. 122 is a comparison of XRPD Diffractogram of S-6-MBPB (tartaric salt) Pattern 7A in various solvents. The diffractogram confirms the crystalline nature of Pattern 7A. The salt screen methods are provided in Examples 42, and 44 and shown in Tables 38, and 40. The x axis measures 2Theta in degrees and the y axis measures intensity measured in arb. units.

FIG. 123 is a comparison of XRPD Diffractogram of S-6-MBPB (succinic salt) Pattern 8A in various solvents. The diffractogram confirms the crystalline nature of Pattern 8A. The salt screen methods are provided in Examples 42, and 44 and shown in Tables 38, and 40. The x axis measures 2Theta in degrees and the y axis measures intensity measured in arb. units.

FIG. 124 is a comparison of XRPD Diffractogram of S-6-MBPB (oxalate salt) Pattern 9A in various solvents. The diffractogram confirms the crystalline nature of Pattern 9A. The salt screen methods are provided in Examples 42, and 44 and shown in Tables 38, and 40. The x axis measures 2Theta in degrees and the y axis measures intensity measured in arb. units.

FIG. 125 is a comparison of XRPD Diffractogram of S-6-MBPB (maleic salt) Pattern 10A in various solvents. The diffractogram confirms the crystalline nature of Pattern 10A. The salt screen methods are provided in Examples 43, and 44 and shown in Tables 39, and 40. The x axis measures 2Theta in degrees and the y axis measures intensity measured in arb. units.

FIG. 126 is a comparison of XRPD Diffractogram of S-6-MBPB (citric salt) Pattern 12A in various solvents. The diffractogram confirms the crystalline nature of Pattern 12A. The salt screen methods are provided in Examples 43, and 44 and shown in Tables 39, and 40. The x axis measures 2Theta in degrees and the y axis measures intensity measured in arb. units.

FIG. 127 is a comparison of XRPD Diffractogram of S-6-MBPB (fumaric salt) Pattern 13B in various solvents. The diffractogram confirms the crystalline nature of Pattern 13B. The salt screen methods are provided in Examples 42, and 44 and shown in Tables 38, and 40. The x axis measures 2Theta in degrees and the y axis measures intensity measured in arb. units.

FIG. 128 is a differential scanning calorimetry (DSC) and thermogravimetric analysis (TGA) of S-6-MBPB Pattern 2A (HBr). The DSC shows a sharp endotherm with onset at ˜154° C., and the TGA shows ˜0.59% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 129 is a differential scanning calorimetry (DSC) and thermogravimetric analysis (TGA) of S-6-MBPB Pattern 4A (H₃PO₄). The DSC shows a sharp endotherm, and the TGA shows ˜10.43% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 130 is a differential scanning calorimetry (DSC) and thermogravimetric analysis (TGA) of S-6-MBPB Pattern 5A (HNO₃). The DSC shows a sharp endotherm (likely melt and decomposition) with onset at ˜96° C., and the TGA shows ˜5.24% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 131 is a differential scanning calorimetry (DSC) and thermogravimetric analysis (TGA) of S-6-MBPB Pattern 7A (tartaric). The DSC shows a sharp endotherm (likely melt) with onset at ˜95° C., and the TGA shows ˜1.61% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 132 is a differential scanning calorimetry (DSC) and thermogravimetric analysis (TGA) of S-6-MBPB Pattern 8A (succinic). The DSC shows a sharp endotherm (likely melt) with onset at ˜90° C., and the TGA shows ˜1.15% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 133 is a differential scanning calorimetry (DSC) and thermogravimetric analysis (TGA) of S-6-MBPB Pattern 9A (oxalate). The DSC shows a sharp endotherm (likely melt) with onset at ˜134° C., and the TGA shows ˜0.93% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 134 is a differential scanning calorimetry (DSC) and thermogravimetric analysis (TGA) of S-6-MBPB Pattern 10A (maleic). The DSC shows a sharp endotherm (likely melt) with onset at ˜82° C., and the TGA shows ˜0.84% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 135 is a differential scanning calorimetry (DSC) and thermogravimetric analysis (TGA) of S-6-MBPB Pattern 12A (citric). The DSC shows a sharp endotherm (likely melt) with onset at ˜104° C., and the TGA shows ˜1.49% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 136 is a differential scanning calorimetry (DSC) and thermogravimetric analysis (TGA) of S-6-MBPB Pattern 13A (fumaric). The DSC shows a sharp endotherm (likely melt) with onset at ˜102° C., and the TGA shows ˜0.60% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 137 is a differential scanning calorimetry (DSC) and thermogravimetric analysis (TGA) of S-6-MBPB Pattern 13B (fumaric). The DSC shows a split endotherm with peaks at ˜108° C. and ˜118° C., and the TGA shows no significant weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 138 is a differential scanning calorimetry (DSC) and thermogravimetric analysis (TGA) of S-6-MBPB Pattern 14A (benzoic). The DSC shows a large, broad endotherm (likely melt and decomposition) with onset at ˜123° C., and the TGA shows no significant weight loss up to 100° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 139 is a differential scanning calorimetry (DSC) and thermogravimetric analysis (TGA) of S-6-MBPB Pattern 15B (salicylic). The DSC shows a small, broad endotherm (likely melt) with onset at ˜40° C., and the TGA shows ˜0.37% weight loss up to 100° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 140 is a XRPD Diffractogram of S-5-MBPB Pattern 1A (Pattern 1AE, S-5-MBPB Enantiomer HCl) in acetone. The diffractogram confirms the crystalline nature of Pattern 1A. The salt screen enantiomer is provided in Examples 47 and Table 43. The x axis measures 2Theta in degrees and the y axis measures intensity measured in arb. units.

FIG. 141 is a XRPD Diffractogram of S-5-MBPB Pattern 2B (Pattern 2BE, S-5-MBPB Enantiomer HBr) in ACN. The diffractogram confirms the crystalline nature of Pattern 2B. The salt screen method is provided in Examples 49 and Table 45. The x axis measures 2Theta in degrees and the y axis measures intensity measured in arb. units.

FIG. 142 is a XRPD Diffractogram of S-5-MBPB Pattern 3A (Pattern 3AE, S-5-MBPB Enantiomer H₃PO₄) in acetone. The diffractogram confirms the crystalline nature of Pattern 3A. The salt screen is provided in Examples 47 and Table 43. The x axis measures 2Theta in degrees and the y axis measures intensity measured in arb. units.

FIG. 143 is a XRPD Diffractogram of S-5-MBPB Pattern 6A (Pattern 6AE, S-5-MBPB Enantiomer succinic) in acetone. The diffractogram confirms the crystalline nature of Pattern 6A. The salt screen is provided in Examples 47 and Table 43. The x axis measures 2Theta in degrees and the y axis measures intensity measured in arb. units.

FIG. 144 is a XRPD Diffractogram of S-5-MBPB Pattern 8A (Pattern 8AE, S-5-MBPB Enantiomer maleic) in acetone. The diffractogram confirms the crystalline nature of Pattern 8A. The salt screen is provided in Examples 47 and Table 43. The x axis measures 2Theta in degrees and the y axis measures intensity measured in arb. units.

FIG. 145 is a XRPD Diffractogram of S-5-MBPB Pattern 9A (Pattern 9AE, S-5-MBPB Enantiomer citric) in ACN. The diffractogram confirms the crystalline nature of Pattern 9A. The salt screen is provided in Examples 49 and Table 45. The x axis measures 2Theta in degrees and the y axis measures intensity measured in arb. units.

FIG. 146 is a XRPD Diffractogram of S-5-MBPB Pattern 10A (Pattern 10AE, S-5-MBPB Enantiomer fumaric) in acetone. The diffractogram confirms the crystalline nature of Pattern 10A. The salt screen method is provided in Examples 47 and Table 43. The x axis measures 2Theta in degrees and the y axis measures intensity measured in arb. units.

FIG. 147 is a comparison of XRPD Diffractogram of S-5-MBPB (HCl salt) Pattern 1A in various solvents. The diffractogram confirms the crystalline nature of Pattern 1A. The salt screen methods are provided in Examples 47, 48 and 49 and shown in Tables 43, 44 and 45. The x axis measures 2Theta in degrees and the y axis measures intensity measured in arb. units.

FIG. 148 is a comparison of XRPD Diffractogram of S-5-MBPB (HBr salt) Patterns 2A and 2B in various solvents. The diffractogram confirms the crystalline nature of Patterns 2A and 2B. The salt screen methods are provided in Examples 47, 48 and 49 and shown in Tables 43, 44 and 45. The x axis measures 2Theta in degrees and the y axis measures intensity measured in arb. units.

FIG. 149 is a comparison of XRPD Diffractogram of S-5-MBPB (H₃PO₄ salt) Pattern 3A in various solvents. The diffractogram confirms the crystalline nature of Pattern 3A. The salt screen methods are provided in Examples 47, 48 and 49 and shown in Tables 43, 44 and 45. The x axis measures 2Theta in degrees and the y axis measures intensity measured in arb. units.

FIG. 150 is a comparison of XRPD Diffractogram of S-5-MBPB (succinic salt) Pattern 6A in acetone. The diffractogram confirms the crystalline nature of Pattern 6A. The salt screen methods are provided in Example 47 and shown in Tables 43. The x axis measures 2Theta in degrees and the y axis measures intensity measured in arb. units.

FIG. 151 is a comparison of XRPD Diffractogram of S-5-MBPB (oxalate salt) Pattern 7A in various solvents. The diffractogram confirms the crystalline nature of Pattern 7A. The salt screen methods are provided in Examples 47, 48 and 49 and shown in Tables 43, 44 and 45. The x axis measures 2Theta in degrees and the y axis measures intensity measured in arb. units.

FIG. 152 is a comparison of XRPD Diffractogram of S-5-MBPB (maleic salt) Pattern 8A in various solvents. The diffractogram confirms the crystalline nature of Pattern 8A. The salt screen methods are provided in Examples 47, 48 and 49 and shown in Tables 43, 44 and 45. The x axis measures 2Theta in degrees and the y axis measures intensity measured in arb. units.

FIG. 153 is a comparison of XRPD Diffractogram of S-5-MBPB (citric salt) Pattern 9A in various solvents. The diffractogram confirms the crystalline nature of Pattern 9A. The salt screen methods are provided in Examples 47, and 49 and shown in Tables 43, and 45. The x axis measures 2Theta in degrees and the y axis measures intensity measured in arb. units.

FIG. 154 is a comparison of XRPD Diffractogram of S-5-MBPB (fumaric salt) Pattern 10A in various solvents. The diffractogram confirms the crystalline nature of Pattern 10A. The salt screen methods are provided in Examples 47, 48 and 49 and shown in Tables 43, 44 and 45. The x axis measures 2Theta in degrees and the y axis measures intensity measured in arb. units.

FIG. 155 is a differential scanning calorimetry (DSC) and thermogravimetric analysis (TGA) of S-5-MBPB Pattern 1A (HCl). The DSC shows a small, broad endotherm with onset at ˜49° C. and a sharp, split endotherm with peaks at ˜132° C. and ˜137° C., and the TGA shows no significant weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 156 is a differential scanning calorimetry (DSC) and thermogravimetric analysis (TGA) of S-5-MBPB Pattern 2B (HBr). The DSC shows a large, broad endotherm (likely melt) with onset at ˜89° C., and the TGA shows ˜0.57% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 157 is a differential scanning calorimetry (DSC) and thermogravimetric analysis (TGA) of S-5-MBPB Pattern 3A (H₃PO₄). The DSC shows a sharp endotherm (likely melt) with onset at ˜180° C., and the TGA shows no significant weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 158 is a differential scanning calorimetry (DSC) thermogram of S-5-MBPB Pattern 6A (succinic). The DSC shows a small, broad endotherm with onset at ˜63° C. and a sharp endotherm (likely melt) with onset at ˜94° C., and the TGA shows ˜0.21% weight loss up to 130° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 159 is a differential scanning calorimetry (DSC) thermogram of S-5-MBPB Pattern 8A (maleic). The DSC shows a sharp endotherm (likely melt) with onset at ˜90° C., and the TGA shows no significant weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 160 is a differential scanning calorimetry (DSC) thermogram of S-5-MBPB Pattern 9A (citric). The DSC shows a sharp endotherm (likely melt) with onset at ˜95° C., and the TGA shows ˜0.81% weight loss up to 130° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 161 is a differential scanning calorimetry (DSC) thermogram of S-5-MBPB Pattern 10A (fumaric). The DSC shows a large, sharp endotherm (likely melt) with onset at ˜102° C., and the TGA shows ˜0.22% weight loss up to 150° C. and decomposition at higher temperatures (>200° C.). The methods used for the DSC/TGA was conducted as described in Example 20 Table 16. The x-axis is temperature measured in degrees Celsius and the y-axis is Weight measured in percentage and Heat flow measured in W/g.

FIG. 162 is a XRPD Diffractogram of R-5-MBPB Pattern 1A (Pattern 1AE, R-5-MBPB Enantiomer HCl). The diffractogram confirms the crystalline nature of Pattern 1A. The liquid-liquid extraction method used to isolate the enantiomer is provided in Examples 50. The x axis measures 2Theta in degrees and the y axis measures intensity measured in arb. units.

FIG. 163 is a XRPD Diffractogram of R-5-MBPB Pattern 3A (Pattern 3AE, R-5-MBPB Enantiomer H₃PO₄) in acetone. The diffractogram confirms the crystalline nature of Pattern 3A. The salt screen method is provided in Examples 52 and Table 48. The x axis measures 2Theta in degrees and the y axis measures intensity measured in arb. units.

FIG. 164 is a XRPD Diffractogram of R-5-MBPB Pattern 8A (Pattern 8AE, R-5-MBPB Enantiomer maleic) in acetone. The diffractogram confirms the crystalline nature of Pattern 8A. The salt screen method is provided in Examples 52 and Table 48. The x axis measures 2Theta in degrees and the y axis measures intensity measured in arb. units.

FIG. 165 is a XRPD Diffractogram of R-5-MBPB Pattern 10A (Pattern 10AE, R-5-MBPB Enantiomer fumaric) in acetone. The diffractogram confirms the crystalline nature of Pattern 10A. The salt screen method is provided in Examples 52 and Table 48. The x axis measures 2Theta in degrees and the y axis measures intensity measured in arb. units.

FIG. 166 is a comparison of XRPD Diffractogram of R-5-MBPB (HCl salt) Pattern 1A. The diffractogram confirms the crystalline nature of Pattern 1A. The liquid-liquid extraction methods to isolate the salt are provided in Example 50 and shown in Table 46. The x axis measures 2Theta in degrees and the y axis measures intensity measured in arb. units.

FIG. 167 is a comparison of XRPD Diffractogram of R-5-MBPB (H₃PO₄ salt) and S-5-MBPB (H₃PO₄ salt) Pattern 3A in acetone. The diffractogram confirms the crystalline nature of Pattern 3A. The salt screen methods are provided in Examples 47, and 51 and shown in Table 43, and 47. The x axis measures 2Theta in degrees and the y axis measures intensity measured in arb. units.

FIG. 168 is a comparison of XRPD Diffractogram of R-5-MBPB (maleic salt) and S-5-MBPB (maleic salt) Pattern 8A in acetone. The diffractogram confirms the crystalline nature of Pattern 8A. The salt screen methods are provided in Examples 47, and 51 and shown in Table 43, and 47. The x axis measures 2Theta in degrees and the y axis measures intensity measured in arb. units.

FIG. 169 is a comparison of XRPD Diffractogram of R-5-MBPB (fumaric salt) and S-5-MBPB (fumaric salt) Pattern 10A in acetone. The diffractogram confirms the crystalline nature of Pattern 10A. The salt screen methods are provided in Examples 47, and 51 and shown in Table 43, and 47. The x axis measures 2Theta in degrees and the y axis measures intensity measured in arb. units.

FIG. 170 is a XRPD Diffractogram of R-6-MBPB Pattern 1A (Pattern 1AE, R-6-MBPB Enantiomer HCl) in ACN. The diffractogram confirms the crystalline nature of Pattern 1A. The liquid-liquid extraction method used to isolate the enantiomer is provided in Examples 54 and Table 50. The x axis measures 2Theta in degrees and the y axis measures intensity measured in arb. units.

FIG. 171 is a XRPD Diffractogram of R-6-MBPB Pattern 2A (Pattern 2AE, R-6-MBPB Enantiomer HBr) in ACN. The diffractogram confirms the crystalline nature of Pattern 2A. The salt screen method is provided in Examples 54 and Table 50. The x axis measures 2Theta in degrees and the y axis measures intensity measured in arb. units.

FIG. 172 is a XRPD Diffractogram of R-6-MBPB Pattern 9A (Pattern 9AE, R-6-MBPB Enantiomer oxalate) in ACN. The diffractogram confirms the crystalline nature of Pattern 1A. The salt screen method is provided in Examples 54 and Table 50. The x axis measures 2Theta in degrees and the y axis measures intensity measured in arb. units.

FIG. 173 is a comparison of XRPD Diffractogram of R-6-MBPB (HCl salt) Pattern 1A. The diffractogram confirms the crystalline nature of Pattern 1A. The salt screen method is provided in Example 53 and shown in Table 49. The x axis measures 2Theta in degrees and the y axis measures intensity measured in arb. units.

FIG. 174 is a comparison of XRPD Diffractogram of R-6-MBPB (HCl salt) and S-6-MBPB (HCl salt) Pattern 1A in ACN. The diffractogram confirms the crystalline nature of Pattern 1A. The salt screen methods are provided in Examples 44, and 54 and shown in Tables 40, and 50. The x axis measures 2Theta in degrees and the y axis measures intensity measured in arb. units.

FIG. 175 is a comparison of XRPD Diffractogram of R-6-MBPB (HBr salt) and S-6-MBPB (HBr salt) Pattern 2A in ACN. The diffractogram confirms the crystalline nature of Pattern 2A. The salt screen methods are provided in Examples 44, and 54 and shown in Tables 40, and 50. The x axis measures 2Theta in degrees and the y axis measures intensity measured in arb. units.

FIG. 176 is a graph showing results from an in vitro rat synaptosome serotonin uptake inhibition assay. The graphs display percent reuptake of [³H]-labeled 5-HT as a function of concentration for RS-5-MBPB, R-5-MBPB, and S-5-MBPB. This data indicates that each tested compound rapidly increases extracellular serotonin by inhibiting reuptake. Details and procedural information for this assay are described in Example 58. The x-axis the log [dose] concentration is measured in Molar units and the y-axis is the [³H]-labeled 5-HT reuptake measured in percent of maximum produced by the comparison releaser.

FIG. 177 is a graph showing results from an in vitro rat synaptosome serotonin release assay. The graphs display [³H]-labeled 5-HT release as a function of concentration for RS-6-MBPB, R-6-MBPB, and S-6-MBPB. These data indicate that each tested compound rapidly increases extracellular serotonin by stimulating release. Details and procedural information for this assay are described in Example 58. The x-axis the log [dose] concentration measured in molar and the y-axis is the [³H]-labeled 5-HT release measured in percent.

FIG. 178 is a graph showing results from in vitro rat synaptosome dopamine and norepinephrine release assays. The graphs display estimated [³H]-labeled dopamine and norepinephrine release as a function of concentration for S-6-MBPB, and R-6-MBPB. Previously presented serotonin results are included for comparison. These data indicate that each tested compound partially increases extracellular norepinephrine by stimulating release, but that the R-enantiomers of 6-MBPB is a dopamine uptake inhibitor. Details and procedural information for this assay are described in Example 58. The x-axis the log [dose] concentration measured in Molar units and the y-axis is the [³H]-labeled 5-HT release measured in percent of maximum produced by the comparison releaser.

FIG. 179 presents non-limiting examples of compounds with new morphic forms and/or salts described herein.

### DETAILED DESCRIPTION OF THE INVENTION

The present invention provides advantageous salts, salt mixtures, and salt morphic forms of benzofuran compounds to treat mental disorders and more generally central nervous system and related disorders as described herein. A benzofuran salt, which may be a solid morphic form, of the present invention can be used for mental enhancement or to treat a mental disorder comprising administering an effective amount of the benzofuran salt or salt morphic form to a host, typically a human, in need thereof. In some embodiments, the benzofuran salts or salt morphic forms or compositions described herein interact with a serotonergic binding site and can exhibit entactogenic properties when administered in an effective amount to a host, typically a human, in need thereof. Thus, a benzofuran salt or salt morphic form described herein can be used as an effective agent for modulating CNS activity and treating CNS disorders described herein.

The embodiments of the invention are presented to meet the goal of assisting persons with mental disorders, who desire mental enhancement, or who suffer from other CNS disorders by providing milder therapeutics that are fast acting and that reduce the properties that decrease the patient experience, are counterproductive to the therapy or are undesirably toxic. One goal of the invention is to provide therapeutic compositions that increase empathy, sympathy, openness and acceptance of oneself and others, which can be taken if necessary as part of therapeutic counseling sessions, when necessary episodically or even consistently, as prescribed by a healthcare provider.

In certain embodiments benzofuran compounds described herein demonstrate permeability properties that indicate the compounds will be fast-acting in humans. This represents a significant improvement over SSRIs, the current standard of care for many CNS and psychological disorders.

The selection of specific advantageous salts, salt mixtures, or morphic forms described herein can increase this fast onset. The slow onset of effects is one of the most pronounced shortcomings of SSRI therapeutics. Thus, in certain embodiments, the salts, salt mixtures, and salt morphic forms of the present invention act as a fast-acting treatment, which represents a significant advance for clinical use. It is advantageous to use a fast-acting therapeutic in a clinical therapeutic setting that typically lasts for one or two hours.

### I. Definitions

When introducing elements of the present invention or the preferred embodiments thereof, the articles “a,” “an,” “the,” and “said” are intended to mean that there are one or more of the elements. The terms “comprising,” “including,” and “having” are intended to be inclusive and not exclusive (i.e., there may be other elements in addition to the recited elements). Thus, the terms “including,” “may include,” and “include,” as used herein mean, and are used interchangeably with, the phrase “including but not limited to.”

Where a range of values is provided, it is understood that the upper and lower limit, and each intervening value between the upper and lower limit of the range is encompassed within the embodiments.

Unless defined otherwise, all technical and scientific terms herein have the meaning as commonly understood by one of ordinary skill in the art to which this invention belongs. In the event there is a plurality of definitions for a term herein, those in this section prevail unless stated otherwise. Further definitions that may assist the reader to understand the disclosed embodiments are as follows, and such definitions may be used to interpret the defined terms, when those terms are used herein. However, the examples given in the definitions are generally non-exhaustive and must not be construed as limiting the invention. It also will be understood that a substituent should comply with chemical bonding rules and steric compatibility constraints in relation to the particular molecule to which it is attached.

The term “CNS disorder” as used herein refers to either a neurological condition (one that is typically treated by a neurologist) or a psychiatric condition (one that is typically treated by a psychiatrist). Neurological disorders are typically those affecting the structure, biochemistry or normal electrical functioning of the brain, spinal cord or other nerves. Psychiatric conditions are more typically thought of as mental disorders, which are primarily abnormalities of thought, feeling or behavior that cause significant distress or impairment of personal functioning. Thus, the disclosed compounds can be used in an effective amount to improve neurological or psychiatric functioning in a patient in need thereof. Neurological indications include, but are not limited to improved neuroplasticity, including treatment of stroke, brain trauma, dementia, and neurodegenerative diseases. Compounds of the current invention can be considered psychoplastogens, that is, small molecules that are able to induce rapid neuroplasticity. For example, in certain embodiments, the disclosed compounds and compositions can be used to improve stuttering and other dyspraxias or to treat Parkinson's disease or schizophrenia.

The term “improving psychiatric function” is intended to include mental health and life conditions that are not traditionally treated by neurologists but sometimes treated by psychiatrists and can also be treated by psychotherapists, life coaches, personal fitness trainers, meditation teachers, counselors, and the like. For example, it is contemplated that the disclosed compounds will allow individuals to effectively contemplate actual or possible experiences that would normally be upsetting or even overwhelming. This includes individuals with fatal illness planning their last days and the disposition of their estate. This also includes couples discussing difficulties in their relationship and how to address them. This also includes individuals who wish to more effectively plan their careers.

The term “inadequate functioning of neurotransmission” is used synonomously with a CNS disorder that adversely affects normal healthy neurotransmission.

The present invention also includes compounds, including enantiomerically enriched compounds and their use, such as 5-MAPB, 6-MAPB, 5-MBPB, 6-MBPB, Bk-5-MAPB, Bk-6-MAPB Formula I, Formula II, Formula III, Formula IV, Formula V, Formula VI, Formula VII, Formula VIII, Formula IX, Formula X, Formula XI, Formula XII, Formula XII, Formula A, Formula B, Formula C, Formula D, Formula E, and Formula F with at least one desired isotopic substitution of an atom, at an amount above the natural abundance of the isotope, i.e., isotopically enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, fluorine and chlorine such as ²H, ³H, ¹¹C, ¹³C, ¹⁴C, ¹⁵N, ¹⁷O, ¹⁸O, ¹⁸F, ³⁶Cl, and respectively. In one non-limiting embodiment, isotopically labelled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is at least 60, 70, 80, 90, 95 or 99% or more enriched in an isotope at any location of interest. In one non-limiting embodiment, deuterium is 90, 95 or 99% enriched at a desired location.

In one non-limiting embodiment, the substitution of a hydrogen atom for a deuterium atom can be provided in a compounds or compositions described herein. In one non-limiting embodiment, the substitution of a hydrogen atom for a deuterium atom occurs within a group selected from any of Q, Z, R¹, R², R³, R⁴, R⁵ or R⁶. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). The compounds of the invention also include isotopically labeled compounds where one or more atoms have an atomic mass different from the atomic mass conventionally found in nature. Examples of isotopes that may be incorporated into the compounds of the invention include ²H, ³H, ¹³C, ¹⁴C, ¹⁵N, ¹⁸O, ¹⁷O, ³¹P, ³²P, ¹⁸F, and ³⁶Cl.

For example, the methyl group on the nitrogen of 5-MAPB, 6-MAPB, 5-MBPB, 6-MBPB, Bk-5-MAPB and Bk-6-MAPB is subject to metabolic removal, which produces pharmacologically active metabolites. In some embodiments, 5-MAPB or 6-MAPB is prepared with deuterium replacing some or all of the three hydrogens on the N-methyl group. In one embodiment, 5-MBPB or 6-MBPB is prepared with deuterium replacing some or all of the three hydrogens on the N-methyl group. In one embodiment, Bk-5-MAPB or Bk-6-MAPB is prepared with deuterium replacing some or all of the three hydrogens on the N-methyl group. This creates a higher activation energy for bond cleavage and a slower formation of the methyl metabolites. Analogously, the two hydrogens on the furan ring may be replaced with one or two deuteriums to decrease metabolic opening of the furan ring and formation of hydroxyl-substituted metabolites.

Similarly, the methyl group on the nitrogen of Formula A, Formula B, Formula C, and Formula D of the invention is subject to metabolic removal, which produces pharmacologically active metabolites. In one embodiment, Formula A or Formula B is prepared with deuterium replacing some or all of the three hydrogens on the N-methyl group. In one embodiment, Formula C or Formula D is prepared with deuterium replacing some or all of the three hydrogens on the N-methyl group. The primary amines of Formula C and Formula D of the invention retain therapeutic effects while presenting a different profile of pharmacological effects. Accordingly, the present disclosure also includes the primary amine variants of Formula C and Formula D, where applicable.

The ethyl group on the nitrogen of Formula E and Formula F is also subject to metabolic removal, which produces pharmacologically active metabolites. In one embodiment, Formula E or Formula F is prepared with deuterium replacing some or all of the three hydrogens on the N-ethyl group. The primary amines of Formula E and Formula F of the invention retain therapeutic effects while presenting a different profile of pharmacological effects. Accordingly, the present disclosure also includes the primary amine variants of Formula E and Formula F, where applicable.

The methyl or ethyl group on the nitrogen where applicable of 5-MAPB, 6-MAPB, 5-MBPB, 6-MBPB, Bk-5-MAPB, Bk-6-MAPB, Formula I, Formula II, Formula III, Formula IV, Formula V, Formula VI, Formula VII, Formula VIII, Formula IX, Formula X, Formula XI, Formula XII, or Formula XII is also subject to metabolic removal, which produces pharmacologically active metabolites. In one embodiment, Formula I, Formula II, Formula III, Formula IV, Formula V, Formula VI, Formula VII, Formula VIII, Formula IX, Formula X, Formula XI, Formula XII, or Formula XII is prepared with deuterium replacing some or all of the three hydrogens on the N-ethyl or N-methyl group. The primary amines of Formula I, Formula II, Formula III, Formula IV, Formula V, Formula VI, Formula VII, Formula VIII, Formula IX, Formula X, Formula XI, Formula XII, and Formula XII of the invention retain therapeutic effects while presenting a different profile of pharmacological effects.

The term “isotopically-labeled” analog refers to an analog that is a “deuterated analog”, a “¹³C-labeled analog,” or a “deuterated/¹³C-labeled analog.” The term “deuterated analog” means a compound described herein, whereby a H-isotope, i.e., hydrogen/protium (¹H), is substituted by a H-isotope, i.e., deuterium (²H). Deuterium substitution can be partial or complete. Partial deuterium substitution means that at least one hydrogen is substituted by at least one deuterium. In certain embodiments, the isotope is at least 60, 70, 80 90, 95 or 99% or more enriched in an isotope at any location of interest. In some embodiments it is deuterium that is 90, 95 or 99% enriched at a desired location. Unless indicated to the contrary, the deuteration is at least 80% at the selected location. Deuteration of the nucleoside can occur at any replaceable hydrogen that provides the desired results.

“Alkyl” in certain specific embodiments refers to a saturated or unsaturated, branched, straight-chain, or cyclic monovalent hydrocarbon radical derived by the removal of one hydrogen atom from a single carbon atom of a parent alkane, alkene or alkyne. Typical alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Alkyl will be understood to include cyclic alkyl radicals such as cyclopropyl, cyclobutyl, and cyclopentyl.

“Alkyl” in certain specific embodiments includes radicals having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 26 carbon atoms, more preferably, from 1 to 10 carbon atoms.

“Halogen” or “halo” means fluoro (F), chloro (Cl), bromo (Br), or iodo (I). For groups containing two or more halogens, such as —CHX₂ or —CX₃, and for example “where X is halogen,” it will be understood that each Y independently will be selected from the group of halogens.

“Hydroxy” means the radical —OH.

“Oxo” means the divalent radical ═O.

“Stereoisomers” includes enantiomers, diastereomers, the components of racemic mixtures, and combinations thereof. Stereoisomers can be prepared or separated as described herein or by using other methods.

“Isomers” includes stereo and geometric isomers, as well as diastereomers. Examples of geometric isomers include cis isomers or trans isomers across a double bond. Other isomers are contemplated among the compounds of the present disclosure. The isomers may be used either in pure form or in admixture with other isomers of the compounds described herein.

“Agonism” refers to the activation of a receptor or enzyme by a modulator, or agonist, to produce a biological response.

“Agonist” refers to a modulator that binds to a receptor or enzyme and activates the receptor to produce a biological response. As a nonlimiting example, “5HT_{1B} agonist” can be used to refer to a compound that exhibits an EC₅₀ with respect to 5HT_{1B} activity of no more than about 10, 25 or even 50 μM. In some embodiments, “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

“DAT to SERT ratio” refers to the tendency of a substance (e.g., a compound or a drug) to increase extracellular dopamine versus increasing extracellular 5-HT concentrations. Higher numbers of this ratio indicate a greater increase of dopamine than serotonin, while lower number indicate an increasing 5-HT more than dopamine. The exact numbers depend on the assay used. The ratio is calculated herein as (DAT EC50)⁻¹/(SERT EC50)⁻¹. Some publications use IC50s for inhibiting uptake instead of EC50s for causing release to calculate this ratio, which will often yield very different results for substances that are monoamine releasers. Thus, it is important to review the numbers in view of the assay and measurement used. While DAT to SERT ratio has been used to predict which compounds will have MDMA-like versus stimulant-like effects, compounds that increase both dopamine and serotonin sometimes have unpredictable effects and some have been proposed as antidepressants, cognitive enhancers, or treatments for substance use disorders. For example, 4-bromomethcathinone (4-BMC, Brephedrone; IUPAC: 1-(4-bromophenyl)-2-(methylamino) propan-1-one) does not have typical psychostimulant effects and has been proposed as a potential antidepressant (Foley and Cozzi. 2003. Drug development research, 60 (4), pp. 252-260). The different therapeutic profiles of these intermediate compounds are believed to be at least partially the result of serotonin inhibiting and modifying the stimulating effects of dopamine (Kimmel et al. 2009. Pharmacology Biochemistry and Behavior, 94 (2), pp. 278-284; Suyama et al. 2019. Psychopharmacology, 236 (3), pp. 1057-1066; Wee et al. 2005. Journal of Pharmacology and Experimental Therapeutics, 313 (2), pp. 848-854).

“IC50” refers to the concentration of a substance (e.g., a compound or a drug) that is required for 50% inhibition of a biological process. For example, IC50 refers to the half maximal (50%) inhibitory concentration (IC) of a substance as determined in a suitable assay. Similarly, EC50 refers to the concentration of a substance that provokes a response halfway between the baseline activity and maximum response. In some instances, an IC50 or EC50 is determined in an in vitro assay system. In some embodiments as used herein, IC50 (or EC50) refers to the concentration of a modulator that is required for 50% inhibition (or excitation) of a receptor, for example, 5HT_{1B}.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., positive allosteric modulator) of a G protein-coupled receptor (e.g., 5-HT_{1B}) are modulators of the receptor.

“Neuroplasticity” refers to the ability of the brain to change its structure and/or function throughout a subject's life. Examples of the changes to the brain include, but are not limited to, the ability to adapt or respond to internal and/or external stimuli, such as due to an injury, and the ability to produce new neurites, dendritic spines, and synapses.

“Treating” or “treatment” of a disease, as used in context, includes (i) inhibiting the disease, i.e., arresting or reducing the development or progression of the disease or its clinical symptoms; or (ii) relieving the disease, i.e., causing regression of the disease or its clinical symptoms. Inhibiting the disease, for example, would include prophylaxis. Hence, one of skill in the art will understand that a therapeutic amount necessary to effect treatment for purposes of this invention will, for example, be an amount that provides for objective indicia of improvement in patients having clinically-diagnosable symptoms. Other such measurements, benefits, and surrogate or clinical endpoints, whether alone or in combination, would be understood to those of ordinary skill.

Examples of salt morphic forms described herein include RS-5-MAPB HCl, RS-5-MAPB HBr, RS-5-MAPB H₃PO₄, RS-5-MAPB oxalic acid, RS-5-MAPB maleic acid, S-5-MAPB HCl, S-5-MAPB HBr, S-5-MAPB H₃PO₄, S-5-MAPB oxalic acid, S-5-MAPB fumaric acid, R-5-MAPB HCl, S-6-MAPB HCl, S-6-MAPB HBr, S-6-MAPB H₃PO₄, and S-6-MAPB oxalic acid, S-BK-5-MAPB HCl, S-BK-5-MAPB HBr, S-BK-5-MAPB H₂SO₄, S-BK-5-MAPB H₃PO₄, S-BK-5-MAPB HNO₃, S-BK-5-MAPB methane sulfonic acid, S-BK-5-MAPB tartaric acid, S-BK-5-MAPB succinic acid, S-BK-5-MAPB oxalic acid, S-BK-5-MAPB maleic acid, S-BK-5-MAPB malic acid, S-BK-5-MAPB citric acid, S-BK-5-MAPB fumaric acid, S-BK-5-MAPB benzoic acid, S-BK-5-MAPB salicylic acid, S-6-MBPB HCl, S-6-MBPB HBr, S-6-MBPB H₂SO₄, S-6-MBPB H₃PO₄, S-6-MBPB HNO₃, S-6-MBPB methane sulfonic acid, S-6-MBPB tartaric acid, S-6-MBPB succinic acid, S-6-MBPB oxalic acid, S-6-MBPB maleic acid, S-6-MBPB malic acid, S-6-MBPB citric acid, S-6-MBPB fumaric acid, S-6-MBPB benzoic acid, S-6-MBPB salicylic acid, S-5-MBPB HCl, S-5-MBPB HBr, S-5-MBPB H₃PO₄, S-5-MBPB HNO₃, S-5-MBPB tartaric acid, S-5-MBPB succinic acid, S-5-MBPB B oxalic acid, S-5-MBPB maleic acid, S-5-MBPB citric acid, S-5-MBPB fumaric acid, R-5-MBPB HCl, R-5-MBPB H₃PO₄, R-5-MBPB maleic acid, R-5-MBPB fumaric acid, R-6-MBPB HCl, R-6-MBPB HBr, and R-6-MBPB oxalate.

### II. COMPOUNDS OF THE PRESENT INVENTION

In certain embodiments a composition is provided that contains one or more benzofuran compounds described herein as one or more advantageous salt morphic forms or salt mixtures described herein as an enantiomerically enriched mixture.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer. An enantiomerically enriched mixture of an R-enantiomer contains at least 55% of the R-enantiomer, and typically at least about 60%, 65%, 70%, 75%, 80%, 85%, 90% or 95% of the R-enantiomer. The specific ratio of S or R enantiomer can be selected for the need of the patient according to the health care specialist to balance the desired effect.

The term enantiomerically enriched mixture as used in this application does not include a racemic mixture and does not include a pure isomer or substantially pure isomer. Notwithstanding, it should be understood that any compound described herein in enantiomerically enriched form can be used as a substantially pure isomer if it achieves the goal of any of the specifically itemized methods of treatment described herein, including but not limited to 5-MAPB, 6-MAPB, 5-MBPB, 6-MBPB, 5-Bk-5-MAPB, 6-Bk-MAPB, Bk-5-MBPB or Bk-6-MBPB.

The chiral carbon typically referred to in this application is the carbon alpha to the amine in the phenylethylamine motif. Of course, the compounds can have additional chiral centers that result in diastereomers. Notwithstanding, in the present application, the primary chiral carbon referred to in the term “enantiomerically enriched” is that carbon alpha to the amine in the provided structures.

In one aspect of the invention, pharmaceutical compositions are provided comprising enantiomerically enriched or enantiomerically substantially pure R-5-MAPB, S-5-MAPB, R-6-MAPB, or S-6-MAPB wherein the pharmaceutical composition was prepared from an advantageous salt or morphic form described herein. In certain embodiments, a pharmaceutical composition is provided that comprises an enantiomerically-enriched mixture of a salt morphic form, morphic salt mixture, or specified salt mixture of R- or S-enantiomer of 5-MAPB or 6-MAPB:

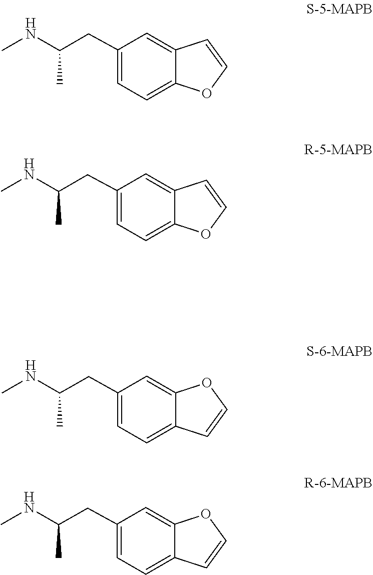

In certain embodiments, isolated enantiomers of the compounds of the present invention show improved binding at the desired receptors and transporters relevant to the goal of treatment for the mental disorder or for mental enhancement.

It is useful to have an S- or R-enantiomerically enriched mixture of these entactogenic compounds that is not a racemic mixture. In certain embodiments enantiomerically enriched mixtures that have a greater amount of the S-enantiomer 5-MAPB or 6-MAPB maximize serotonin-receptor-dependent therapeutic effects, whereas the enantiomerically enriched R-enantiomer of 5-MAPB or 6-MAPB maximize nicotinic-receptor-dependent therapeutic effects. Therefore, one aspect of the present invention is a balanced mixture of S-5-MAPB and R-5-MAPB or a balanced mixture of S-6-MAPB and R-6-MAPB that achieves a predetermined combination of serotonin-receptor-dependent therapeutic effects and nicotinic-receptor-dependent or dopaminergic therapeutic effects. The effect can be modulated as desired for optimal therapeutic effect.

Accordingly, in one embodiment, an enantiomerically enriched mixture of a salt morphic form, morphic salt mixture, or specified salt mixture of S-5-MAPB or an enantiomerically enriched mixture of a salt morphic form, morphic salt mixture, or specified salt mixture of S-6-MAPB maximize serotonin-receptor-dependent therapeutic effects and minimize unwanted nicotinic effects or dopaminergic effects when administered to a host in need thereof, for example a mammal, including a human.

In another embodiment, an enantiomerically enriched mixture of a salt morphic form, morphic salt mixture, or specified salt mixture of R-5-MAPB or an enantiomerically enriched mixture of a salt morphic form, morphic salt mixture, or specified salt mixture of R-6-MAPB maximize nicotinic-receptor-dependent or dopaminergic-receptor dependent therapeutic effects while minimizing unwanted effects, when administered to a host in need thereof, including a mammal, for example, a human.

Non-limiting examples of unwanted effects that can be minimized by carefully selecting the balance of enantiomers include hallucinogenic effects, psychoactive effects (such as excess stimulation or sedation), physiological effects (such as transient hypertension or appetite suppression), toxic effects (such as to the brain or liver), effects contributing to abuse liability (such as euphoria or dopamine release), and/or other side effects.

Enantiomerically enriched mixtures of 5-MAPB that are non-racemic have a relatively greater amount of some therapeutic effects (such as emotional openness) while having lesser effects associated with abuse liability (such as perceptible ‘good drug effects’ which can lead to abuse versus openness, which leads to more tranquility and peace). Therefore, one aspect of the present invention is a balanced mixture of a salt morphic form, morphic salt mixture, or specified salt mixture of S-5-MAPB and R-5-MAPB or a balanced mixture of a salt morphic form, morphic salt mixture, or specified salt mixture of S-6-MAPB and R-6-MAPB that achieves a predetermined combination of emotional therapeutic effects and perceptible mood effects. The effect can be modulated as desired for optimal therapeutic effect.

Accordingly, in one embodiment, an enantiomerically enriched mixture of a salt morphic form, morphic salt mixture, or specified salt mixture of S-5-MAPB or an enantiomerically enriched mixture of a salt morphic form, morphic salt mixture, or specified salt mixture of S-6-MAPB balances emotional openness and perceptible mood effects when administered to a host in need thereof, for example a mammal, including a human.

In certain embodiments, it is preferred to have an S- or R-enantiomerically enriched mixture. In certain embodiments enantiomerically enriched mixtures are provided that have a greater amount of the R-enantiomer of 5-MAPB or 6-MAPB maximize nicotinic-receptor-dependent therapeutic effects and that enantiomerically enriched mixtures that have a greater amount of the S-enantiomer 5-MAPB or 6-MAPB maximize serotonin-receptor-dependent therapeutic effects. Therefore, one aspect of the present invention is a balanced mixture of S-5-MAPB and R-5-MAPB or a balanced mixture of S-6-MAPB and R-6-MAPB that achieves a predetermined combination of serotonin-receptor-dependent therapeutic effects and nicotinic-receptor-dependent therapeutic effects.

Accordingly, in one embodiment, an enantiomerically enriched mixture of S-5-MAPB or an enantiomerically enriched mixture of S-6-MAPB maximize serotonin-receptor-dependent therapeutic effects and minimized unwanted nicotinic effects when administered to a host in need thereof, for example a mammal, including a human.

In another embodiment, an enantiomerically enriched mixture of R-5-MAPB or an enantiomerically enriched mixture of R-6-MAPB maximize nicotinic-receptor-dependent therapeutic effects while minimizing unwanted effects, when administered to a host in need thereof, including a mammal, for example, a human.

The present invention also provides new medical uses for a salt morphic form, morphic salt mixture, or specified salt mixture of a compound of Formulas I-X and enantiomerically enriched compositions of 5-MAPB, 6-MAPB, 5-MBPB, 6-MBPB, 5-Bk-5-MAPB, 6-Bk-MAPB, Bk-5-MBPB, Bk-6-MBPB, or the compounds of Formulas A-F by administering an effective amount to a patient such as a human to treat a CNS disorder including but not limited to, the treatment of depression, dysthymia, anxiety, generalized anxiety, social anxiety, panic, adjustment disorders, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism or dissociative disorders or any other disorder described herein, including in the Background.

Several of the benzofuran derivatives of the current invention are direct 5-HT_{1B} agonists. Very few substances are known that are 5-HT_{1B} agonists and also 5-HT releasers and of those, some show significant toxicities. For example, m-chlorophenylpiperazine (mCPP) is one example but is anxiogenic and induces headaches, limiting any clinical use. MDMA itself does not bind to the 5-HT_{1B} (Ray. 2010. PloS one, 5 (2), e9019). 5-HT_{1B} agonism is noteworthy because indirect stimulation of these receptors, secondary to elevated extracellular serotonin, has been hypothesized to be required for the prosocial effects of MDMA (Heifets et al. 2019. Science translational medicine, 11 (522)), while other aspects of entactogen effects have been attributed to monoamine release (e.g., Luethi & Liechti. 2020. Archives of Toxicology, 94 (4), 1085-1133). Thus, the unique ratios of 5-HT_{1B} stimulation and monoamine release displayed by the disclosed compounds enable different profiles of therapeutic effects that cannot be achieved by MDMA or other known entactogens.

The compounds of the present invention show a 5-HT selectivity pattern that is important to therapeutic use. Various subtypes of 5-HT receptor can induce different felt experiences on a patient. Agonism of the 5-HT_{2A} receptor can cause feelings of fear and hallucinations, but agonism of 5-HT_{1B} is believed to be tied to the pro-social effects of entactogens. Various subtypes of 5-HT receptor can also contribute to different toxicity risks for a patient. Administration of MDMA and other serotonergic drugs is associated with elevated acute risk of hyponatremia. It is known that stimulation of 5-HT₂ receptors is an important trigger of release of antidiuretic hormone (Iovino et a. Current pharmaceutical design 18, no. 30 (2012): 4714-4724).

Enantiomeric compositions of the present invention can be selected to be poor agonists of 5-HT_{2A}, but exhibit activity toward 5-HT_{1B}. For example, as described in the non-limiting illustrative Example 6, the majority of the compounds do not exhibit 5-HT_{2A} agonist activity but do exhibit 5-HT_{1B} agonist activity in the range of about 5 to 0.0005 μM, or 3 to 0.10 μM. Importantly, 5-HT_{1B} agonist activity effect occurs through direct action on the receptor, rather than as an indirect consequence of serotonin release. This is an unexpected because this property has not been observed in an entactogen, including MDMA, before. In one embodiment, the selectivity toward the 5-HT_{1B} receptor over 5-HT_{2A} receptor allows for a more relaxed and therapeutically productive experience for the patient undergoing treatment with a compound of the present invention.

The unique ratios of 5-HT_{1B} stimulation and 5-HT release displayed by the disclosed compounds enable different profiles of therapeutic effects and side effects that may not be achieved by MDMA or other known entactogens. An undesirable effect of releasing 5-HT can be hyponatremia or loss of appetite. Drugs such as d-fenfluramine that release 5-HT by interacting with SERT and thereby increase agonism of all serotonin receptors have been used as anorectics. Similarly, MDMA is known to acutely suppress appetite (see, e.g., Vollenweider et al. Neuropsychopharmacology 19, no. 4 (1998): 241-251).

Accordingly, as described in the non-limiting illustrative Example 9, the enantiomeric compositions of the present invention have ability to release 5-HT with potencies (EC50s) in the range of approximately 5 to 0.001 UM or 1.3 to 0.003 μM. In another embodiment, therefore, the selectivity toward the 5-HT_{1B} receptor over SERT-mediated 5-HT release allows for a therapeutically productive experience for the patient undergoing treatment with a compound of the present invention with fewer other side effects from serotonin release, such as loss of appetite or risk of hyponatremia.

The present invention also includes a salt morphic form, morphic salt mixture, or specified salt mixture of compounds with beneficial selectivity profiles for neurotransmitter transporters. The balance of weakly activating NET (to reduce cardiovascular toxicity risk) and having a relatively low DAT to SERT ratio (to increase therapeutic effect relative to addictive liability) is a desirable feature of an entactogenic therapy displayed by the compounds and compositions of the present invention.

### Embodiments of R

In certain embodiments R is hydrogen.

In certain embodiments R is hydroxyl.

### Embodiments of R^{A}

In certain embodiments R^{A} is —CH₃.

In certain embodiments R^{A} is —CH₂Y.

In certain embodiments R^{A} is —CHY₂.

In certain embodiments R^{A} is —CY₃.

In certain embodiments R^{A} is —CH₂CH₃.

In certain embodiments R^{A} is —CH₂CH₂Y.

In certain embodiments R^{A} is —CH₂CHY₂.

In certain embodiments R^{A} is —CH₂CY₃.

In certain embodiments R^{A} is —CH₂OH.

In certain embodiments R^{A} is —CH₂CH₂OH.

### Embodiments of Q

In certain embodiments Q is

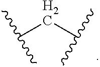 [SMILES: *C*]

In certain embodiments Q is

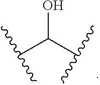 [SMILES: *C(*)O]

In certain embodiments Q is

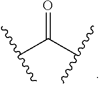 [SMILES: *C(*)=O]

In certain embodiments Q is

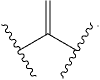 [SMILES: *C(*)=C]

In certain embodiments Q is

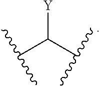

### Embodiments of Y

In certain embodiments Y is F.

In certain embodiments Y is Cl.

### Embodiments of “alkyl”

Unless otherwise specifically referenced “alkyl” is a branched, straight chain, or cyclic saturated aliphatic hydrocarbon group. In certain embodiments, the alkyl from 1 to about 6 carbon atoms, from 1 to about 4 carbon atoms, or from 1 to 3 carbon atoms. In certain embodiments, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl. In certain embodiments “alkyl” is a C₁-C₆alkyl, C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In certain embodiments when a term is used that includes “alk” it should be understood that “cycloalkyl” or “carbocyclic” can be considered part of the definition, unless unambiguously excluded by the context. For example, and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

### Morphic Forms of S-6-MAPB

### HCl Salt

In certain aspects the invention provides S-6-MAPB as an HCl salt for therapeutic uses. In certain embodiments the S-6-MAPB HCl salt is a stable morphic form denoted Pattern 1A.
a. In certain embodiments S-6-MAPB HCl Pattern 1A is characterized by an XRPD pattern with three or more peaks selected from 17.7, 15.8, 21.3, 24.4, 25.6, 28.8, 31.7, and 35.0+/−0.4° 2theta.
b. In certain embodiments S-6-MAPB HCl Pattern 1A is characterized by an XRPD pattern with four or more peaks selected from 17.7, 15.8, 21.3, 24.4, 25.6, 28.8, 31.7, and 35.0+/−0.4° 2theta.
c. In certain embodiments S-6-MAPB HCl Pattern 1A is characterized by an XRPD pattern with five or more peaks selected from 17.7, 15.8, 21.3, 24.4, 25.6, 28.8, 31.7, and 35.0+/−0.4° 2theta.
d. In certain embodiments S-6-MAPB HCl Pattern 1A is characterized by an XRPD pattern with six or more peaks selected from 17.7, 15.8, 21.3, 24.4, 25.6, 28.8, 31.7, and 35.0+/−0.4° 2theta.
e. In certain embodiments S-6-MAPB HCl Pattern 1A is characterized by an XRPD pattern with seven or more peaks selected from 17.7, 15.8, 21.3, 24.4, 25.6, 28.8, 31.7, and 35.0+/−0.4° 2theta.
f. In certain embodiments S-6-MAPB HCl Pattern 1A is characterized by any one of embodiments a-e wherein the XRPD pattern includes a peak at 17.7+/−0.4° 2theta.
g. In certain embodiments S-6-MAPB HCl Pattern 1A is characterized by any one of embodiments a-f wherein the XRPD pattern includes a peak at 21.3+/−0.4° 2theta.
h. In certain embodiments S-6-MAPB HCl Pattern 1A is characterized by any one of embodiments a-g wherein the XRPD pattern includes a peak at 15.8+/−0.4° 2theta.
i. In certain embodiments S-6-MAPB HCl Pattern 1A is characterized by any one of embodiments a-h wherein the XRPD pattern includes a peak at 28.8+/−0.4° 2theta.
j. In certain embodiments S-6-MAPB HCl Pattern 1A is characterized by any one of embodiments a-i wherein the XRPD pattern includes a peak at 24.4+/−0.4° 2theta.
k. In certain embodiments S-6-MAPB HCl Pattern 1A is characterized by any one of embodiments a-j wherein the peaks are within +/−0.3° 2theta.
l. In certain embodiments S-6-MAPB HCl Pattern 1A is characterized by any one of embodiments a-j wherein the peaks are within +/−0.2° 2theta.
m. In certain embodiments S-6-MAPB HCl Pattern 1A is characterized by any one of embodiments a-l wherein at least three of the recited peaks have a relative peak intensity of at least 5%.
n. In certain embodiments S-6-MAPB HCl Pattern 1A is characterized by any one of embodiments a-l wherein at least three of the recited peaks have a relative peak intensity of at least 10%.
o. In certain embodiments S-6-MAPB HCl Pattern 1A is characterized by any one of embodiments a-l wherein at least four of the recited peaks have a relative peak intensity of at least 5%.
p. In certain embodiments S-6-MAPB HCl Pattern 1A is characterized by any one of embodiments a-l wherein at least four of the recited peaks have a relative peak intensity of at least 10%.
q. In certain embodiments S-6-MAPB HCl Pattern 1A is characterized by any one of embodiments a-l wherein at least five of the recited peaks have a relative peak intensity of at least 5%.
r. In certain embodiments S-6-MAPB HCl Pattern 1A is characterized by any one of embodiments a-l wherein at least six of the recited peaks have a relative peak intensity of at least 5%.

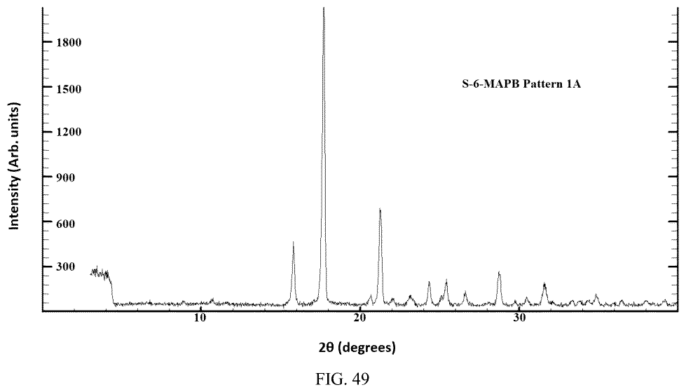

In certain embodiments Pattern 1A is characterized by the XRPD diffractogram in FIG. 49 and/or the DSC graph shown in FIG. 57.

### HBr Salt

In certain aspects the invention provides S-6-MAPB as an HBr salt. In certain embodiments the S-6-MAPB HBr salt is a stable morphic form denoted Pattern 2A.
a. In certain embodiments S-6-MAPB HBr Pattern 2A is characterized by an XRPD pattern with three or more peaks selected from 16.3, 17.7, 18.2, 21.1, 21.4, 22.0, 22.6, 24.1, 25.2, 26.6, 27.1, 28.2, 28.5, 28.8, 29.2, 30.0, 30.5, 31.2, 31.3, 32.2, 32.4, 33.0, 33.5, 33.9, 35.1, 36.2, 38.0, 38.6, and 38.8+/−0.4° 2theta.
b. In certain embodiments S-6-MAPB HBr Pattern 2A is characterized by an XRPD pattern with four or more peaks selected from 16.3, 17.7, 18.2, 21.1, 21.4, 22.0, 22.6, 24.1, 25.2, 26.6, 27.1, 28.2, 28.5, 28.8, 29.2, 30.0, 30.5, 31.2, 31.3, 32.2, 32.4, 33.0, 33.5, 33.9, 35.1, 36.2, 38.0, 38.6, and 38.8+/−0.4° 2theta.
c. In certain embodiments S-6-MAPB HBr Pattern 2A is characterized by an XRPD pattern with five or more peaks selected from 16.3, 17.7, 18.2, 21.1, 21.4, 22.0, 22.6, 24.1, 25.2, 26.6, 27.1, 28.2, 28.5, 28.8, 29.2, 30.0, 30.5, 31.2, 31.3, 32.2, 32.4, 33.0, 33.5, 33.9, 35.1, 36.2, 38.0, 38.6, and 38.8+/−0.4° 2theta.
d. In certain embodiments S-6-MAPB HBr Pattern 2A is characterized by an XRPD pattern with six or more peaks selected from 16.3, 17.7, 18.2, 21.1, 21.4, 22.0, 22.6, 24.1, 25.2, 26.6, 27.1, 28.2, 28.5, 28.8, 29.2, 30.0, 30.5, 31.2, 31.3, 32.2, 32.4, 33.0, 33.5, 33.9, 35.1, 36.2, 38.0, 38.6, and 38.8+/−0.4° 2theta.
e. In certain embodiments S-6-MAPB HBr Pattern 2A is characterized by an XRPD pattern with seven or more peaks selected from 16.3, 17.7, 18.2, 21.1, 21.4, 22.0, 22.6, 24.1, 25.2, 26.6, 27.1, 28.2, 28.5, 28.8, 29.2, 30.0, 30.5, 31.2, 31.3, 32.2, 32.4, 33.0, 33.5, 33.9, 35.1, 36.2, 38.0, 38.6, and 38.8+/−0.4° 2theta.
f. In certain embodiments S-6-MAPB HBr Pattern 2A is characterized by any one of embodiments a-e wherein the XRPD pattern includes a peak at 21.4+/−0.4° 2theta.
g. In certain embodiments S-6-MAPB HBr Pattern 2A is characterized by any one of embodiments a-f wherein the XRPD pattern includes a peak at 25.2+/−0.4° 2theta.
h. In certain embodiments S-6-MAPB HBr Pattern 2A is characterized by any one of embodiments a-g wherein the XRPD pattern includes a peak at 32.2+/−0.4° 2theta.
i. In certain embodiments S-6-MAPB HBr Pattern 2A is characterized by any one of embodiments a-h wherein the XRPD pattern includes a peak at 28.8+/−0.4° 2theta.
j. In certain embodiments S-6-MAPB HBr Pattern 2A is characterized by any one of embodiments a-i wherein the XRPD pattern includes a peak at 29.2+/−0.4° 2theta.
k. In certain embodiments S-6-MAPB HBr Pattern 2A is characterized by any one of embodiments a-j wherein the peaks are within +/−0.3° 2theta.
l. In certain embodiments S-6-MAPB HBr Pattern 2A is characterized by any one of embodiments a-j wherein the peaks are within +/−0.2° 2theta.
m. In certain embodiments S-6-MAPB HBr Pattern 2A is characterized by any one of embodiments a-l wherein at least three of the recited peaks have a relative peak intensity of at least 10%.
n. In certain embodiments S-6-MAPB HBr Pattern 2A is characterized by any one of embodiments a-l wherein at least three of the recited peaks have a relative peak intensity of at least 20%.
o. In certain embodiments S-6-MAPB HBr Pattern 2A is characterized by any one of embodiments a-l wherein at least four of the recited peaks have a relative peak intensity of at least 10%.
p. In certain embodiments S-6-MAPB HBr Pattern 2A is characterized by any one of embodiments a-l wherein at least four of the recited peaks have a relative peak intensity of at least 20%.
q. In certain embodiments S-6-MAPB HBr Pattern 2A is characterized by any one of embodiments a-l wherein at least five of the recited peaks have a relative peak intensity of at least 10%.
r. In certain embodiments S-6-MAPB HBr Pattern 2A is characterized by any one of embodiments a-l wherein at least six of the recited peaks have a relative peak intensity of at least 20%.

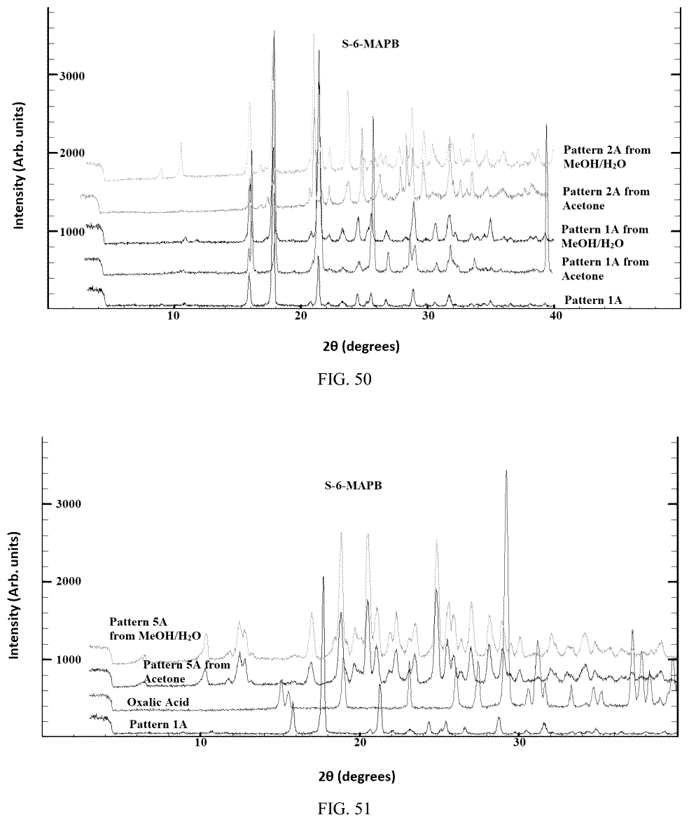

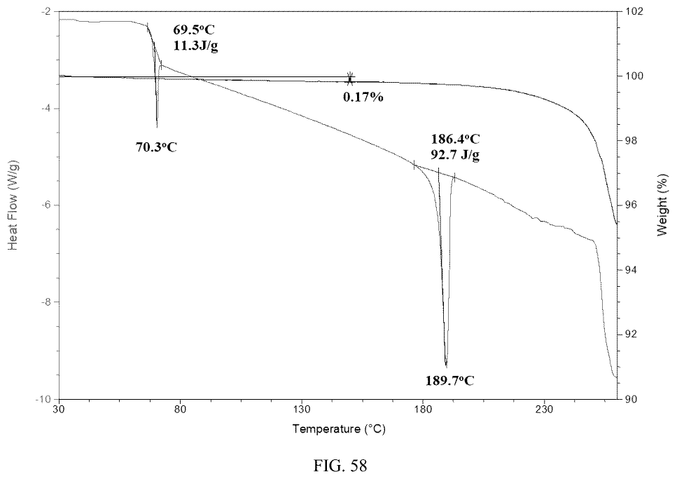

In certain embodiments S-6-MAPB HBr Pattern 2A is characterized by the XRPD diffractogram in FIG. 50 and/or the DSC graph shown in FIG. 58.

H₃PO₄ Salt

In certain aspects the invention provides S-6-MAPB as an H₃PO₄ salt. In certain embodiments the S-6-MAPB H₃PO₄ salt is a stable morphic form denoted Pattern 3A or Pattern 3B.
a. In certain embodiments S-6-MAPB H₃PO₄ Pattern 3A is characterized by an XRPD pattern with three or more peaks selected from 13.5, 15.1, 17.0, 17.8, 18.4, 19.3, 19.8, 20.1, 20.6, 21.5, 22.2, 22.6, 24.5, 25.6, 26.6, 26.8, 27.2, 27.6, 29.5, 32.9, 35.1, 35.3, 37.8, and 39.6+/−0.4° 2theta.
b. In certain embodiments S-6-MAPB H₃PO₄ Pattern 3A is characterized by an XRPD pattern with four or more peaks selected from 13.5, 15.1, 17.0, 17.8, 18.4, 19.3, 19.8, 20.1, 20.6, 21.5, 22.2, 22.6, 24.5, 25.6, 26.6, 26.8, 27.2, 27.6, 29.5, 32.9, 35.1, 35.3, 37.8, and 39.6+/−0.4° 2theta.
c. In certain embodiments S-6-MAPB H₃PO₄ Pattern 3A is characterized by an XRPD pattern with five or more peaks selected from 13.5, 15.1, 17.0, 17.8, 18.4, 19.3, 19.8, 20.1, 20.6, 21.5, 22.2, 22.6, 24.5, 25.6, 26.6, 26.8, 27.2, 27.6, 29.5, 32.9, 35.1, 35.3, 37.8, and 39.6+/−0.4° 2theta.
d. In certain embodiments S-6-MAPB H₃PO₄ Pattern 3A is characterized by an XRPD pattern with six or more peaks selected from 13.5, 15.1, 17.0, 17.8, 18.4, 19.3, 19.8, 20.1, 20.6, 21.5, 22.2, 22.6, 24.5, 25.6, 26.6, 26.8, 27.2, 27.6, 29.5, 32.9, 35.1, 35.3, 37.8, and 39.6+/−0.4° 2theta.
e. In certain embodiments S-6-MAPB H₃PO₄ Pattern 3A is characterized by an XRPD pattern with seven or more peaks selected from 13.5, 15.1, 17.0, 17.8, 18.4, 19.3, 19.8, 20.1, 20.6, 21.5, 22.2, 22.6, 24.5, 25.6, 26.6, 26.8, 27.2, 27.6, 29.5, 32.9, 35.1, 35.3, 37.8, and 39.6+/−0.4° 2theta.
f. In certain embodiments S-6-MAPB H₃PO₄ Pattern 3A is characterized by any one of embodiments a-e wherein the XRPD pattern includes a peak at 22.2+/−0.4° 2theta.
g. In certain embodiments S-6-MAPB H₃PO₄ Pattern 3A is characterized by any one of embodiments a-f wherein the XRPD pattern includes a peak at 13.5+/−0.4° 2theta.
h. In certain embodiments S-6-MAPB H₃PO₄ Pattern 3A is characterized by any one of embodiments a-g wherein the XRPD pattern includes a peak at 27.6+/−0.4° 2theta.
i. In certain embodiments S-6-MAPB H₃PO₄ Pattern 3A is characterized by any one of embodiments a-h wherein the XRPD pattern includes a peak at 17.8+/−0.4° 2theta.
j. In certain embodiments S-6-MAPB H₃PO₄ Pattern 3A is characterized by any one of embodiments a-i wherein the XRPD pattern includes a peak at 27.2 and/or 20.1+/−0.4° 2theta.
k. In certain embodiments S-6-MAPB H₃PO₄ Pattern 3A is characterized by any one of embodiments a-j wherein the peaks are within +/−0.3° 2theta.
l. In certain embodiments S-6-MAPB H₃PO₄ Pattern 3A is characterized by any one of embodiments a-j wherein the peaks are within +/−0.2° 2theta.
m. In certain embodiments S-6-MAPB H₃PO₄ Pattern 3A is characterized by any one of embodiments a-l wherein at least three of the recited peaks have a relative peak intensity of at least 10%.
n. In certain embodiments S-6-MAPB H₃PO₄ Pattern 3A is characterized by any one of embodiments a-l wherein at least three of the recited peaks have a relative peak intensity of at least 20%.
o. In certain embodiments S-6-MAPB H₃PO₄ Pattern 3A is characterized by any one of embodiments a-l wherein at least four of the recited peaks have a relative peak intensity of at least 10%.
p. In certain embodiments S-6-MAPB H₃PO₄ Pattern 3A is characterized by any one of embodiments a-l wherein at least four of the recited peaks have a relative peak intensity of at least 20%.
q. In certain embodiments S-6-MAPB H₃PO₄ Pattern 3A is characterized by any one of embodiments a-l wherein at least five of the recited peaks have a relative peak intensity of at least 10%.
r. In certain embodiments S-6-MAPB H₃PO₄ Pattern 3A is characterized by any one of embodiments a-l wherein at least six of the recited peaks have a relative peak intensity of at least 20%.

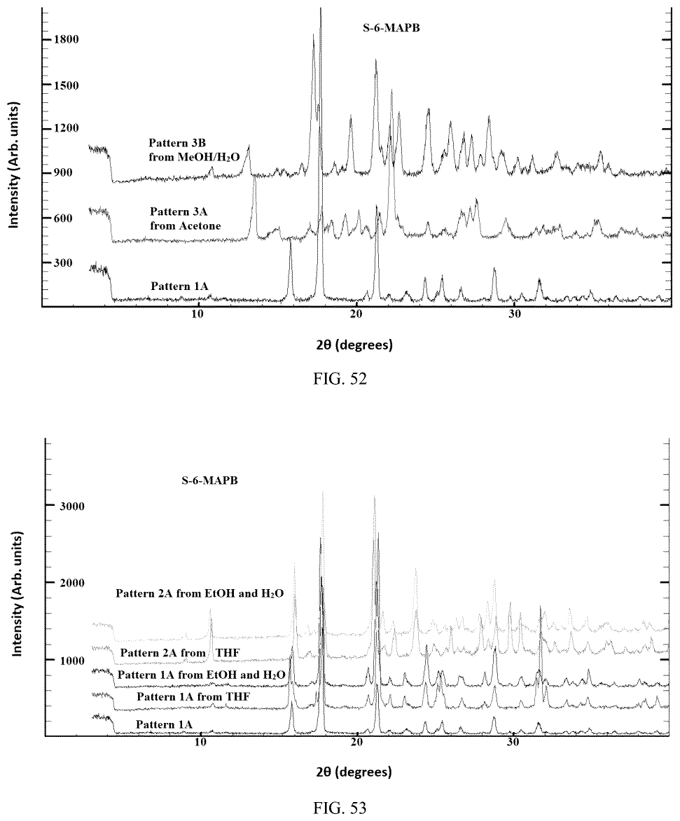

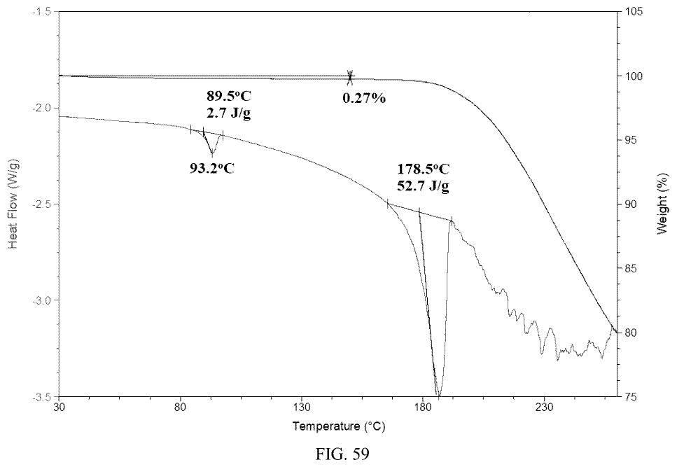

In certain embodiments S-6-MAPB H₃PO₄ Pattern 3A is characterized by the XRPD diffractogram in FIG. 52 and/or the DSC graph shown in FIG. 59.
a. In certain embodiments S-6-MAPB H₃PO₄ Pattern 3B is characterized by an XRPD pattern with three or more peaks selected from 10.8, 13.1, 16.5, 17.3, 17.6, 18.6, 18.7, 19.6, 21.2, 21.6, 22.1, 22.7, 24.6, 25.4, 25.5, 26.0, 26.1, 26.6, 26.7, 26.8, 27.3, 27.9, 28.4, 28.7, 29.2, 29.4, 30.0, 30.2, 30.7, 31.2, 32.6, 32.7, 34.0, 34.4, 34.6, 34.7, 35.5, 35.9, 36.0, 36.8, 37.6, 39.4, and 39.7+/−0.4° 2theta.
b. In certain embodiments S-6-MAPB H₃PO₄ Pattern 3B is characterized by an XRPD pattern with four or more peaks selected from 10.8, 13.1, 16.5, 17.3, 17.6, 18.6, 18.7, 19.6, 21.2, 21.6, 22.1, 22.7, 24.6, 25.4, 25.5, 26.0, 26.1, 26.6, 26.7, 26.8, 27.3, 27.9, 28.4, 28.7, 29.2, 29.4, 30.0, 30.2, 30.7, 31.2, 32.6, 32.7, 34.0, 34.4, 34.6, 34.7, 35.5, 35.9, 36.0, 36.8, 37.6, 39.4, and 39.7+/−0.4° 2theta.
c. In certain embodiments S-6-MAPB H₃PO₄ Pattern 3B is characterized by an XRPD pattern with five or more peaks selected from 10.8, 13.1, 16.5, 17.3, 17.6, 18.6, 18.7, 19.6, 21.2, 21.6, 22.1, 22.7, 24.6, 25.4, 25.5, 26.0, 26.1, 26.6, 26.7, 26.8, 27.3, 27.9, 28.4, 28.7, 29.2, 29.4, 30.0, 30.2, 30.7, 31.2, 32.6, 32.7, 34.0, 34.4, 34.6, 34.7, 35.5, 35.9, 36.0, 36.8, 37.6, 39.4, and 39.7+/−0.4° 2theta.
d. In certain embodiments S-6-MAPB H₃PO₄ Pattern 3B is characterized by an XRPD pattern with six or more peaks selected from 10.8, 13.1, 16.5, 17.3, 17.6, 18.6, 18.7, 19.6, 21.2, 21.6, 22.1, 22.7, 24.6, 25.4, 25.5, 26.0, 26.1, 26.6, 26.7, 26.8, 27.3, 27.9, 28.4, 28.7, 29.2, 29.4, 30.0, 30.2, 30.7, 31.2, 32.6, 32.7, 34.0, 34.4, 34.6, 34.7, 35.5, 35.9, 36.0, 36.8, 37.6, 39.4, and 39.7+/−0.4° 2theta.
e. In certain embodiments S-6-MAPB H₃PO₄ Pattern 3B is characterized by an XRPD pattern with seven or more peaks selected from 10.8, 13.1, 16.5, 17.3, 17.6, 18.6, 18.7, 19.6, 21.2, 21.6, 22.1, 22.7, 24.6, 25.4, 25.5, 26.0, 26.1, 26.6, 26.7, 26.8, 27.3, 27.9, 28.4, 28.7, 29.2, 29.4, 30.0, 30.2, 30.7, 31.2, 32.6, 32.7, 34.0, 34.4, 34.6, 34.7, 35.5, 35.9, 36.0, 36.8, 37.6, 39.4, and 39.7+/−0.4° 2theta.
f. In certain embodiments S-6-MAPB H₃PO₄ Pattern 3B is characterized by any one of embodiments a-e wherein the XRPD pattern includes a peak at 17.3+/−0.4° 2theta.
g. In certain embodiments S-6-MAPB H₃PO₄ Pattern 3B is characterized by any one of embodiments a-f wherein the XRPD pattern includes a peak at 21.2+/−0.4° 2theta.
h. In certain embodiments S-6-MAPB H₃PO₄ Pattern 3B is characterized by any one of embodiments a-g wherein the XRPD pattern includes a peak at 17.6+/−0.4° 2theta.
i. In certain embodiments S-6-MAPB H₃PO₄ Pattern 3B is characterized by any one of embodiments a-h wherein the XRPD pattern includes a peak at 24.6+/−0.4° 2theta.
j. In certain embodiments S-6-MAPB H₃PO₄ Pattern 3B is characterized by any one of embodiments a-i wherein the XRPD pattern includes a peak at 22.7+/−0.4° 2theta.
k. In certain embodiments S-6-MAPB H₃PO₄ Pattern 3B is characterized by any one of embodiments a-j wherein the peaks are within +/−0.3° 2theta.
l. In certain embodiments S-6-MAPB H₃PO₄ Pattern 3B is characterized by any one of embodiments a-j wherein the peaks are within +/−0.2° 2theta.
m. In certain embodiments S-6-MAPB H₃PO₄ Pattern 3B is characterized by any one of embodiments a-l wherein at least three of the recited peaks have a relative peak intensity of at least 10%.
n. In certain embodiments S-6-MAPB H₃PO₄ Pattern 3B is characterized by any one of embodiments a-l wherein at least three of the recited peaks have a relative peak intensity of at least 20%.
o. In certain embodiments S-6-MAPB H₃PO₄ Pattern 3B is characterized by any one of embodiments a-l wherein at least four of the recited peaks have a relative peak intensity of at least 10%.
p. In certain embodiments S-6-MAPB H₃PO₄ Pattern 3B is characterized by any one of embodiments a-l wherein at least four of the recited peaks have a relative peak intensity of at least 20%.
q. In certain embodiments S-6-MAPB H₃PO₄ Pattern 3B is characterized by any one of embodiments a-l wherein at least five of the recited peaks have a relative peak intensity of at least 10%.
r. In certain embodiments S-6-MAPB H₃PO₄ Pattern 3B is characterized by any one of embodiments a-l wherein at least six of the recited peaks have a relative peak intensity of at least 20%.

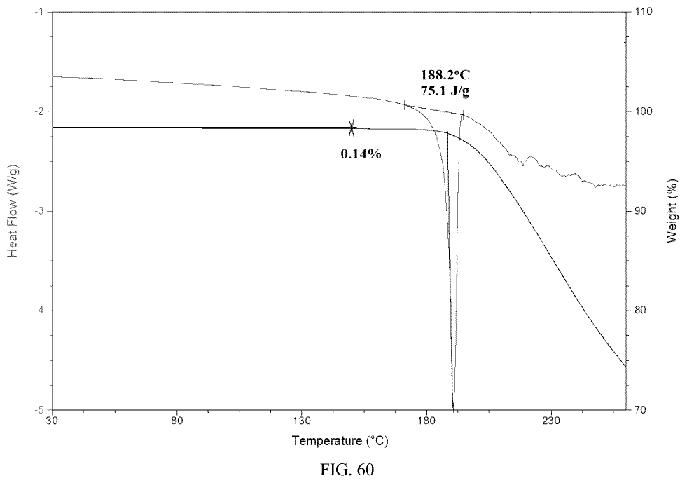

In certain embodiments S-6-MAPB H₃PO₄ Pattern 3B is characterized by the XRPD diffractogram in FIG. 52 and/or the DSC graph shown in FIG. 60.

### Oxalic Salt

In certain aspects the invention provides S-6-MAPB as an oxalate salt. In certain embodiments the S-6-MAPB oxalate salt is a stable morphic form denoted Pattern 5A.
a. In certain embodiments S-6-MAPB oxalate Pattern 5A is characterized by an XRPD pattern with three or more peaks selected from 10.3, 12.4, 12.8, 17.0, 18.8, 19.7, 20.5, 21.1, 21.9, 22.3, 23.1, 23.5, 24.9, 25.5, 25.9, 26.4, 27.0, 28.2, 29.1, 29.5, 30.1, 32.0, 32.3, 34.2, 34.8, 35.8, 37.2, and 39.0+/−0.4° 2theta.
b. In certain embodiments S-6-MAPB oxalate Pattern 5A is characterized by an XRPD pattern with four or more peaks selected from 10.3, 12.4, 12.8, 17.0, 18.8, 19.7, 20.5, 21.1, 21.9, 22.3, 23.1, 23.5, 24.9, 25.5, 25.9, 26.4, 27.0, 28.2, 29.1, 29.5, 30.1, 32.0, 32.3, 34.2, 34.8, 35.8, 37.2, and 39.0+/−0.4° 2theta.
c. In certain embodiments S-6-MAPB oxalate Pattern 5A is characterized by an XRPD pattern with five or more peaks selected from 10.3, 12.4, 12.8, 17.0, 18.8, 19.7, 20.5, 21.1, 21.9, 22.3, 23.1, 23.5, 24.9, 25.5, 25.9, 26.4, 27.0, 28.2, 29.1, 29.5, 30.1, 32.0, 32.3, 34.2, 34.8, 35.8, 37.2, and 39.0+/−0.4° 2theta.
d. In certain embodiments S-6-MAPB oxalate Pattern 5A is characterized by an XRPD pattern with six or more peaks selected from 10.3, 12.4, 12.8, 17.0, 18.8, 19.7, 20.5, 21.1, 21.9, 22.3, 23.1, 23.5, 24.9, 25.5, 25.9, 26.4, 27.0, 28.2, 29.1, 29.5, 30.1, 32.0, 32.3, 34.2, 34.8, 35.8, 37.2, and 39.0+/−0.4° 2theta.
e. In certain embodiments S-6-MAPB oxalate Pattern 5A is characterized by an XRPD pattern with seven or more peaks selected from 10.3, 12.4, 12.8, 17.0, 18.8, 19.7, 20.5, 21.1, 21.9, 22.3, 23.1, 23.5, 24.9, 25.5, 25.9, 26.4, 27.0, 28.2, 29.1, 29.5, 30.1, 32.0, 32.3, 34.2, 34.8, 35.8, 37.2, and 39.0+/−0.4° 2theta.
f. In certain embodiments S-6-MAPB oxalate Pattern 5A is characterized by any one of embodiments a-e wherein the XRPD pattern includes a peak at 18.8+/−0.4° 2theta.
g. In certain embodiments S-6-MAPB oxalate Pattern 5A is characterized by any one of embodiments a-f wherein the XRPD pattern includes a peak at 20.5+/−0.4° 2theta.
h. In certain embodiments S-6-MAPB oxalate Pattern 5A is characterized by any one of embodiments a-g wherein the XRPD pattern includes a peak at 24.9+/−0.4° 2theta.
i. In certain embodiments S-6-MAPB oxalate Pattern 5A is characterized by any one of embodiments a-h wherein the XRPD pattern includes a peak at 25.6+/−0.4° 2theta.
j. In certain embodiments S-6-MAPB oxalate Pattern 5A is characterized by any one of embodiments a-i wherein the XRPD pattern includes a peak at 27.0+/−0.4° 2theta.
k. In certain embodiments S-6-MAPB oxalate Pattern 5A is characterized by any one of embodiments a-j wherein the peaks are within +/−0.3° 2theta.
l. In certain embodiments S-6-MAPB oxalate Pattern 5A is characterized by any one of embodiments a-j wherein the peaks are within +/−0.2° 2theta.
m. In certain embodiments S-6-MAPB oxalate Pattern 5A is characterized by any one of embodiments a-l wherein at least three of the recited peaks have a relative peak intensity of at least 10%.
n. In certain embodiments S-6-MAPB oxalate Pattern 5A is characterized by any one of embodiments a-l wherein at least three of the recited peaks have a relative peak intensity of at least 20%.
o. In certain embodiments S-6-MAPB oxalate Pattern 5A is characterized by any one of embodiments a-l wherein at least four of the recited peaks have a relative peak intensity of at least 10%.
p. In certain embodiments S-6-MAPB oxalate Pattern 5A is characterized by any one of embodiments a-l wherein at least four of the recited peaks have a relative peak intensity of at least 20%.
q. In certain embodiments S-6-MAPB oxalate Pattern 5A is characterized by any one of embodiments a-l wherein at least five of the recited peaks have a relative peak intensity of at least 10%.
r. In certain embodiments S-6-MAPB oxalate Pattern 5A is characterized by any one of embodiments a-l wherein at least six of the recited peaks have a relative peak intensity of at least 20%.

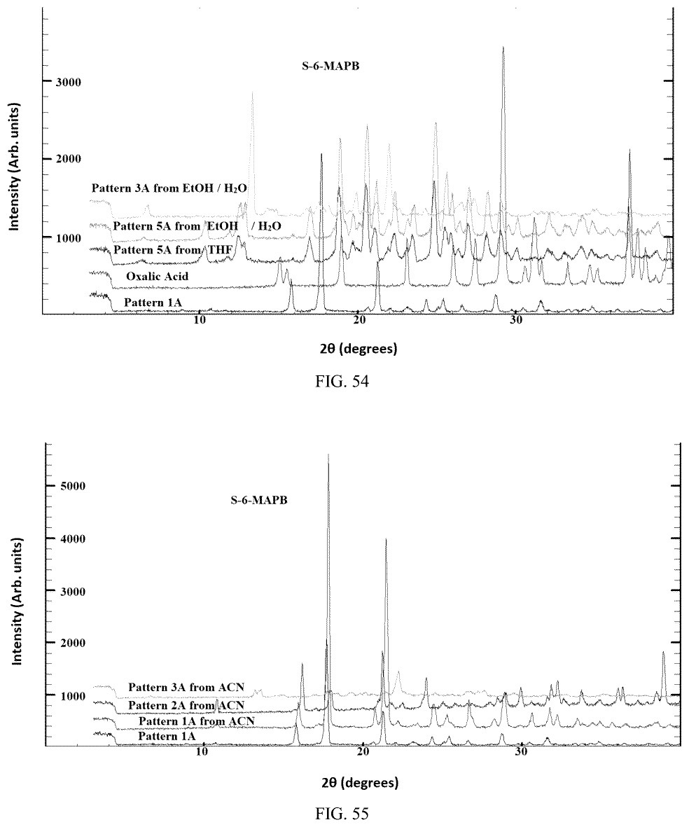

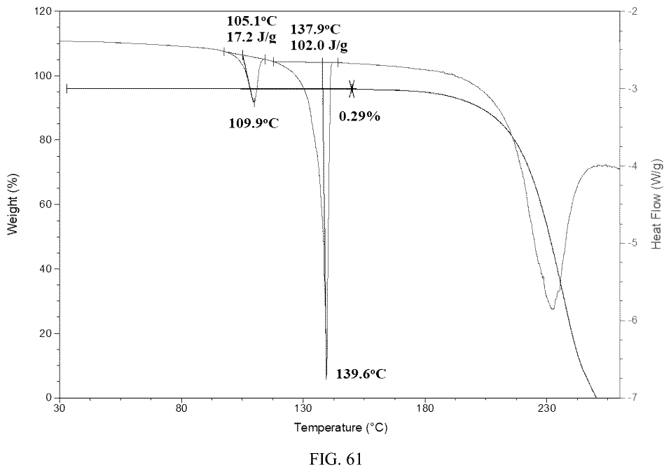

In certain embodiments S-6-MAPB oxalate Pattern 5A is characterized by the XRPD diffractogram in FIG. 54 and/or the DSC graph shown in FIG. 61.

### Morphic Forms of R/S-5-MAPB

### HCl Salt

In certain aspects the invention provides R/S-5-MAPB as an HCl salt for therapeutic uses. In certain embodiments the R/S-5-MAPB HCl salt is a stable morphic form denoted Pattern 1A.
a. In certain embodiments R/S-5-MAPB HCl Pattern 1A is characterized by an XRPD pattern with three or more peaks selected from 13.2, 15.1, 16.9, 17.9, 19.2, 20.8, 22.5, 24.4, 25.4, 25.8, 26.2, 27.6, and 31.6+/−0.4° 2theta.
b. In certain embodiments R/S-5-MAPB HCl Pattern 1A is characterized by an XRPD pattern with four or more peaks selected from 13.2, 15.1, 16.9, 17.9, 19.2, 20.8, 22.5, 24.4, 25.4, 25.8, 26.2, 27.6, and 31.6+/−0.4° 2theta.
c. In certain embodiments R/S-5-MAPB HCl Pattern 1A is characterized by an XRPD pattern with five or more peaks selected from 13.2, 15.1, 16.9, 17.9, 19.2, 20.8, 22.5, 24.4, 25.4, 25.8, 26.2, 27.6, and 31.6+/−0.4° 2theta.
d. In certain embodiments R/S-5-MAPB HCl Pattern 1A is characterized by an XRPD pattern with six or more peaks selected from 13.2, 15.1, 16.9, 17.9, 19.2, 20.8, 22.5, 24.4, 25.4, 25.8, 26.2, 27.6, and 31.6+/−0.4° 2theta.
e. In certain embodiments R/S-5-MAPB HCl Pattern 1A is characterized by an XRPD pattern with seven or more peaks selected from 13.2, 15.1, 16.9, 17.9, 19.2, 20.8, 22.5, 24.4, 25.4, 25.8, 26.2, 27.6, and 31.6+/−0.4° 2theta.
f. In certain embodiments R/S-5-MAPB HCl Pattern 1A is characterized by any one of embodiments a-e wherein the XRPD pattern includes a peak at 19.2+/−0.4° 2theta.
g. In certain embodiments R/S-5-MAPB HCl Pattern 1A is characterized by any one of embodiments a-f wherein the XRPD pattern includes a peak at 27.6+/−0.4° 2theta.
h. In certain embodiments R/S-5-MAPB HCl Pattern 1A is characterized by any one of embodiments a-g wherein the XRPD pattern includes a peak at 15.1+/−0.4° 2theta.
i. In certain embodiments R/S-5-MAPB HCl Pattern 1A is characterized by any one of embodiments a-h wherein the XRPD pattern includes a peak at 17.9+/−0.4° 2theta.
j. In certain embodiments R/S-5-MAPB HCl Pattern 1A is characterized by any one of embodiments a-i wherein the XRPD pattern includes a peak at 25.4 or 22.5+/−0.4° 2theta.
k. In certain embodiments R/S-5-MAPB HCl Pattern 1A is characterized by any one of embodiments a-j wherein the peaks are within +/−0.3° 2theta.
l. In certain embodiments R/S-5-MAPB HCl Pattern 1A is characterized by any one of embodiments a-j wherein the peaks are within +/−0.2° 2theta.
m. In certain embodiments R/S-5-MAPB HCl Pattern 1A is characterized by any one of embodiments a-l wherein at least three of the recited peaks have a relative peak intensity of at least 5%.
n. In certain embodiments R/S-5-MAPB HCl Pattern 1A is characterized by any one of embodiments a-l wherein at least three of the recited peaks have a relative peak intensity of at least 10%.
o. In certain embodiments R/S-5-MAPB HCl Pattern 1A is characterized by any one of embodiments a-l wherein at least four of the recited peaks have a relative peak intensity of at least 5%.
p. In certain embodiments R/S-5-MAPB HCl Pattern 1A is characterized by any one of embodiments a-l wherein at least four of the recited peaks have a relative peak intensity of at least 10%.
q. In certain embodiments R/S-5-MAPB HCl Pattern 1A is characterized by any one of embodiments a-l wherein at least five of the recited peaks have a relative peak intensity of at least 5%.
r. In certain embodiments R/S-5-MAPB HCl Pattern 1A is characterized by any one of embodiments a-l wherein at least six of the recited peaks have a relative peak intensity of at least 5%.

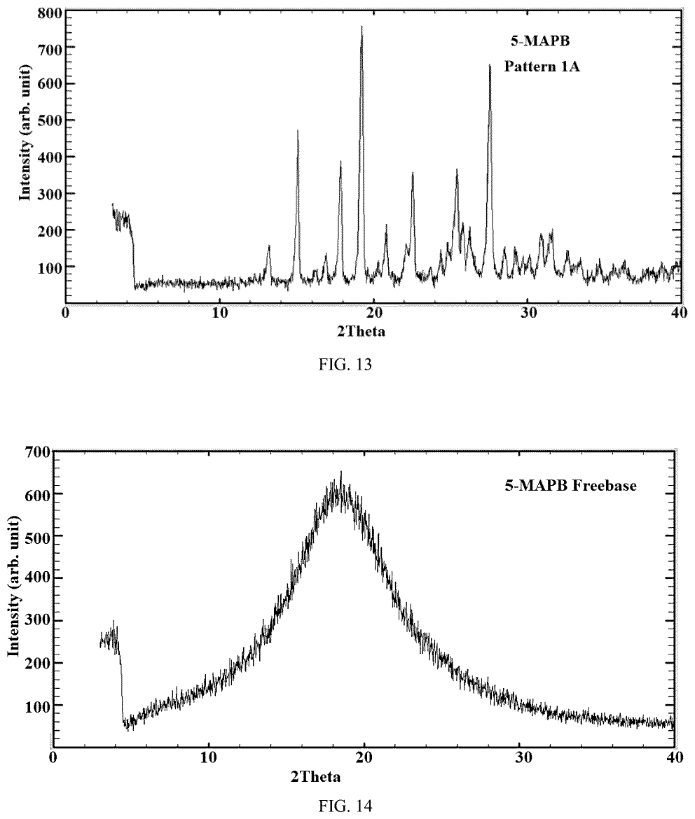

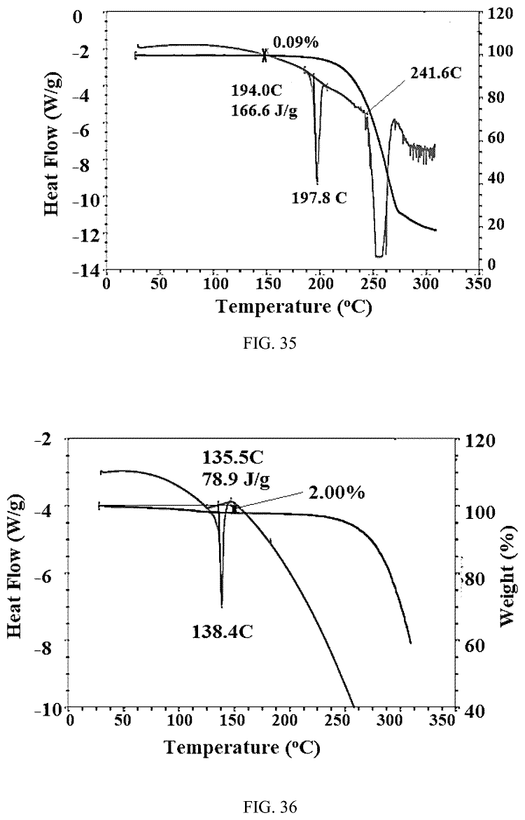

In certain embodiments R/S-5-MAPB HCl Pattern 1A is characterized by the XRPD diffractogram in FIG. 13 and/or the DSC graph shown in FIG. 35.

### HBr Salt

In certain aspects the invention provides R/S-5-MAPB as an HBr salt. In certain embodiments the R/S-5-MAPB HBr salt is a stable morphic form denoted Pattern 2A.
a. In certain embodiments R/S-5-MAPB HBr Pattern 2A is characterized by an XRPD pattern with three or more peaks selected from 11.9, 14.1, 16.2, 17.1, 22.2, 23.2, 23.7, 24.3, 26.9, 28.2, and 35.6+/−0.4° 2theta.
b. In certain embodiments R/S-5-MAPB HBr Pattern 2A is characterized by an XRPD pattern with four or more peaks selected from 11.9, 14.1, 16.2, 17.1, 22.2, 23.2, 23.7, 24.3, 26.9, 28.2, and 35.6+/−0.4° 2theta.
c. In certain embodiments R/S-5-MAPB HBr Pattern 2A is characterized by an XRPD pattern with five or more peaks selected from 11.9, 14.1, 16.2, 17.1, 22.2, 23.2, 23.7, 24.3, 26.9, 28.2, and 35.6+/−0.4° 2theta.
d. In certain embodiments R/S-5-MAPB HBr Pattern 2A is characterized by an XRPD pattern with six or more peaks selected from 11.9, 14.1, 16.2, 17.1, 22.2, 23.2, 23.7, 24.3, 26.9, 28.2, and 35.6+/−0.4° 2theta.
e. In certain embodiments R/S-5-MAPB HBr Pattern 2A is characterized by an XRPD pattern with seven or more peaks selected from 11.9, 14.1, 16.2, 17.1, 22.2, 23.2, 23.7, 24.3, 26.9, 28.2, and 35.6+/−0.4° 2theta.
f. In certain embodiments R/S-5-MAPB HBr Pattern 2A is characterized by any one of embodiments a-e wherein the XRPD pattern includes a peak at 23.7+/−0.4° 2theta.
g. In certain embodiments R/S-5-MAPB HBr Pattern 2A is characterized by any one of embodiments a-f wherein the XRPD pattern includes a peak at 28.2+/−0.4° 2theta.
h. In certain embodiments R/S-5-MAPB HBr Pattern 2A is characterized by any one of embodiments a-g wherein the XRPD pattern includes a peak at 16.2+/−0.4° 2theta.
i. In certain embodiments R/S-5-MAPB HBr Pattern 2A is characterized by any one of embodiments a-h wherein the XRPD pattern includes a peak at 14.1+/−0.4° 2theta.
j. In certain embodiments R/S-5-MAPB HBr Pattern 2A is characterized by any one of embodiments a-i wherein the XRPD pattern includes a peak at 26.9 or 35.6+/−0.4° 2theta.
k. In certain embodiments R/S-5-MAPB HBr Pattern 2A is characterized by any one of embodiments a-j wherein the peaks are within +/−0.3° 2theta.
l. In certain embodiments R/S-5-MAPB HBr Pattern 2A is characterized by any one of embodiments a-j wherein the peaks are within +/−0.2° 2theta.
m. In certain embodiments R/S-5-MAPB HBr Pattern 2A is characterized by any one of embodiments a-l wherein at least three of the recited peaks have a relative peak intensity of at least 10%.
n. In certain embodiments R/S-5-MAPB HBr Pattern 2A is characterized by any one of embodiments a-l wherein at least three of the recited peaks have a relative peak intensity of at least 20%.
o. In certain embodiments R/S-5-MAPB HBr Pattern 2A is characterized by any one of embodiments a-l wherein at least four of the recited peaks have a relative peak intensity of at least 10%.
p. In certain embodiments R/S-5-MAPB HBr Pattern 2A is characterized by any one of embodiments a-l wherein at least four of the recited peaks have a relative peak intensity of at least 20%.
q. In certain embodiments R/S-5-MAPB HBr Pattern 2A is characterized by any one of embodiments a-l wherein at least five of the recited peaks have a relative peak intensity of at least 10%.
r. In certain embodiments R/S-5-MAPB HBr Pattern 2A is characterized by any one of embodiments a-l wherein at least six of the recited peaks have a relative peak intensity of at least 20%.

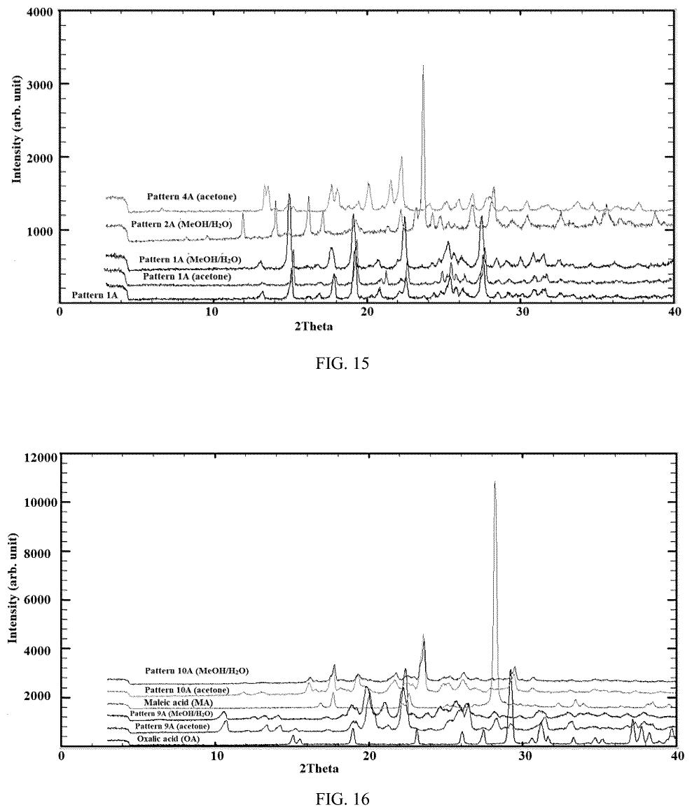

In certain embodiments R/S-5-MAPB HBr Pattern 2A is characterized by the XRPD diffractogram in FIG. 15 and/or the DSC graph shown in FIG. 36.

H₃PO₄ Salt

In certain aspects the invention provides R/S-5-MAPB as an H₃PO₄ salt. In certain embodiments the R/S-5-MAPB H₃PO₄ salt is a stable morphic form denoted Pattern 4A, Pattern 4B, or Pattern 4C.
a. In certain embodiments R/S-5-MAPB H₃PO₄ Pattern 4A is characterized by an XRPD pattern with three or more peaks selected from 13.3, 13.6, 17.7, 18.1, 19.5, 20.1, 21.6, 22.3, 24.1, 25.2, 26.0, 26.9, 27.8, 30.4, 34.7, and 37.7+/−0.4° 2theta.
b. In certain embodiments R/S-5-MAPB H₃PO₄ Pattern 4A is characterized by an XRPD pattern with four or more peaks selected from 13.3, 13.6, 17.7, 18.1, 19.5, 20.1, 21.6, 22.3, 24.1, 25.2, 26.0, 26.9, 27.8, 30.4, 34.7, and 37.7+/−0.4° 2theta.
c. In certain embodiments R/S-5-MAPB H₃PO₄ Pattern 4A is characterized by an XRPD pattern with five or more peaks selected from 13.3, 13.6, 17.7, 18.1, 19.5, 20.1, 21.6, 22.3, 24.1, 25.2, 26.0, 26.9, 27.8, 30.4, 34.7, and 37.7+/−0.4° 2theta.
d. In certain embodiments R/S-5-MAPB H₃PO₄ Pattern 4A is characterized by an XRPD pattern with six or more peaks selected from 13.3, 13.6, 17.7, 18.1, 19.5, 20.1, 21.6, 22.3, 24.1, 25.2, 26.0, 26.9, 27.8, 30.4, 34.7, and 37.7+/−0.4° 2theta.
e. In certain embodiments R/S-5-MAPB H₃PO₄ Pattern 4A is characterized by an XRPD pattern with seven or more peaks selected from 13.3, 13.6, 17.7, 18.1, 19.5, 20.1, 21.6, 22.3, 24.1, 25.2, 26.0, 26.9, 27.8, 30.4, 34.7, and 37.7+/−0.4° 2theta.
f. In certain embodiments R/S-5-MAPB H₃PO₄ Pattern 4A is characterized by any one of embodiments a-e wherein the XRPD pattern includes a peak at 22.3+/−0.4° 2theta.
g. In certain embodiments R/S-5-MAPB H₃PO₄ Pattern 4A is characterized by any one of embodiments a-f wherein the XRPD pattern includes a peak at 21.6+/−0.4° 2theta.
h. In certain embodiments R/S-5-MAPB H₃PO₄ Pattern 4A is characterized by any one of embodiments a-g wherein the XRPD pattern includes a peak at 20.1+/−0.4° 2theta.
i. In certain embodiments R/S-5-MAPB H₃PO₄ Pattern 4A is characterized by any one of embodiments a-h wherein the XRPD pattern includes a peak at 13.6+/−0.4° 2theta.
j. In certain embodiments R/S-5-MAPB H₃PO₄ Pattern 4A is characterized by any one of embodiments a-i wherein the XRPD pattern includes a peak at 13.3 and/or 17.7+/−0.4° 2theta.
k. In certain embodiments R/S-5-MAPB H₃PO₄ Pattern 4A is characterized by any one of embodiments a-j wherein the peaks are within +/−0.3° 2theta.
l. In certain embodiments R/S-5-MAPB H₃PO₄ Pattern 4A is characterized by any one of embodiments a-j wherein the peaks are within +/−0.2° 2theta.
m. In certain embodiments R/S-5-MAPB H₃PO₄ Pattern 4A is characterized by any one of embodiments a-l wherein at least three of the recited peaks have a relative peak intensity of at least 10%.
n. In certain embodiments R/S-5-MAPB H₃PO₄ Pattern 4A is characterized by any one of embodiments a-l wherein at least three of the recited peaks have a relative peak intensity of at least 20%.
o. In certain embodiments R/S-5-MAPB H₃PO₄ Pattern 4A is characterized by any one of embodiments a-l wherein at least four of the recited peaks have a relative peak intensity of at least 10%.
p. In certain embodiments R/S-5-MAPB H₃PO₄ Pattern 4A is characterized by any one of embodiments a-l wherein at least four of the recited peaks have a relative peak intensity of at least 20%.
q. In certain embodiments R/S-5-MAPB H₃PO₄ Pattern 4A is characterized by any one of embodiments a-l wherein at least five of the recited peaks have a relative peak intensity of at least 10%.
r. In certain embodiments R/S-5-MAPB H₃PO₄ Pattern 4A is characterized by any one of embodiments a-l wherein at least six of the recited peaks have a relative peak intensity of at least 20%.

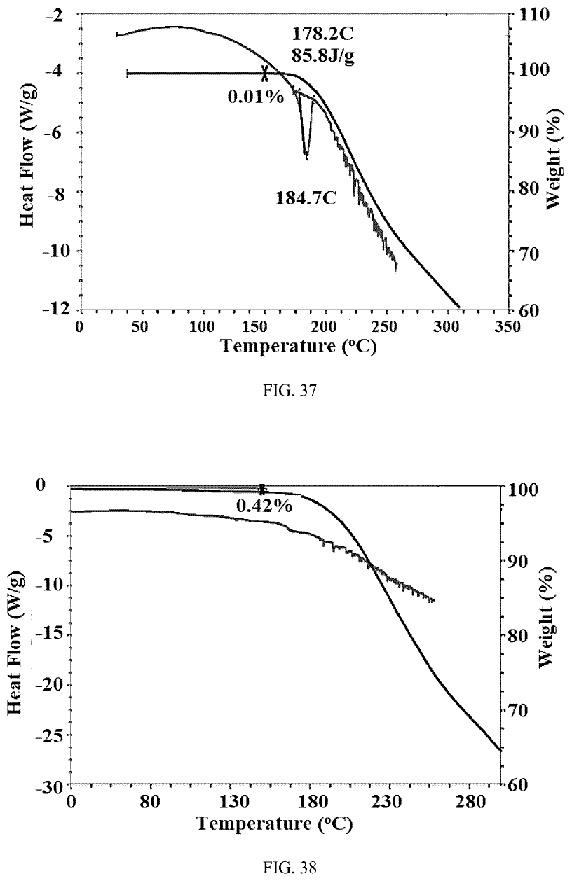

In certain embodiments R/S-5-MAPB H₃PO₄ Pattern 4A is characterized by the XRPD diffractogram in FIG. 15 and/or the DSC graph shown in FIG. 37.
a. In certain embodiments R/S-5-MAPB H₃PO₄ Pattern 4B is characterized by an XRPD pattern with three or more peaks selected from 12.3, 13.8, 17.0, 18.7, 20.9, 21.8, 23.4, 24.5, 27.1, and 28.2+/−0.4° 2theta.
b. In certain embodiments R/S-5-MAPB H₃PO₄ Pattern 4B is characterized by an XRPD pattern with four or more peaks selected from 12.3, 13.8, 17.0, 18.7, 20.9, 21.8, 23.4, 24.5, 27.1, and 28.2+/−0.4° 2theta.
c. In certain embodiments R/S-5-MAPB H₃PO₄ Pattern 4B is characterized by an XRPD pattern with five or more peaks selected from 12.3, 13.8, 17.0, 18.7, 20.9, 21.8, 23.4, 24.5, 27.1, and 28.2+/−0.4° 2theta.
d. In certain embodiments R/S-5-MAPB H₃PO₄ Pattern 4B is characterized by an XRPD pattern with six or more peaks selected from 12.3, 13.8, 17.0, 18.7, 20.9, 21.8, 23.4, 24.5, 27.1, and 28.2+/−0.4° 2theta.
e. In certain embodiments R/S-5-MAPB H₃PO₄ Pattern 4B is characterized by an XRPD pattern with seven or more peaks selected from 12.3, 13.8, 17.0, 18.7, 20.9, 21.8, 23.4, 24.5, 27.1, and 28.2+/−0.4° 2theta.
f. In certain embodiments R/S-5-MAPB H₃PO₄ Pattern 4B is characterized by any one of embodiments a-e wherein the XRPD pattern includes a peak at 12.3+/−0.4° 2theta.
g. In certain embodiments R/S-5-MAPB H₃PO₄ Pattern 4B is characterized by any one of embodiments a-f wherein the XRPD pattern includes a peak at 13.8+/−0.4° 2theta.
h. In certain embodiments R/S-5-MAPB H₃PO₄ Pattern 4B is characterized by any one of embodiments a-g wherein the XRPD pattern includes a peak at 17.0+/−0.4° 2theta.
i. In certain embodiments R/S-5-MAPB H₃PO₄ Pattern 4B is characterized by any one of embodiments a-h wherein the XRPD pattern includes a peak at 18.7+/−0.4° 2theta.
j. In certain embodiments R/S-5-MAPB H₃PO₄ Pattern 4B is characterized by any one of embodiments a-i wherein the XRPD pattern includes a peak at 20.9+/−0.4° 2theta.
k. In certain embodiments R/S-5-MAPB H₃PO₄ Pattern 4B is characterized by any one of embodiments a-j wherein the peaks are within +/−0.3° 2theta.
l. In certain embodiments R/S-5-MAPB H₃PO₄ Pattern 4B is characterized by any one of embodiments a-j wherein the peaks are within +/−0.2° 2theta.
m. In certain embodiments R/S-5-MAPB H₃PO₄ Pattern 4B is characterized by any one of embodiments a-l wherein at least three of the recited peaks have a relative peak intensity of at least 10%.
n. In certain embodiments R/S-5-MAPB H₃PO₄ Pattern 4B is characterized by any one of embodiments a-l wherein at least three of the recited peaks have a relative peak intensity of at least 20%.
o. In certain embodiments R/S-5-MAPB H₃PO₄ Pattern 4B is characterized by any one of embodiments a-l wherein at least four of the recited peaks have a relative peak intensity of at least 10%.
p. In certain embodiments R/S-5-MAPB H₃PO₄ Pattern 4B is characterized by any one of embodiments a-l wherein at least four of the recited peaks have a relative peak intensity of at least 20%.
q. In certain embodiments R/S-5-MAPB H₃PO₄ Pattern 4B is characterized by any one of embodiments a-l wherein at least five of the recited peaks have a relative peak intensity of at least 10%.
r. In certain embodiments R/S-5-MAPB H₃PO₄ Pattern 4B is characterized by any one of embodiments a-l wherein at least six of the recited peaks have a relative peak intensity of at least 20%.

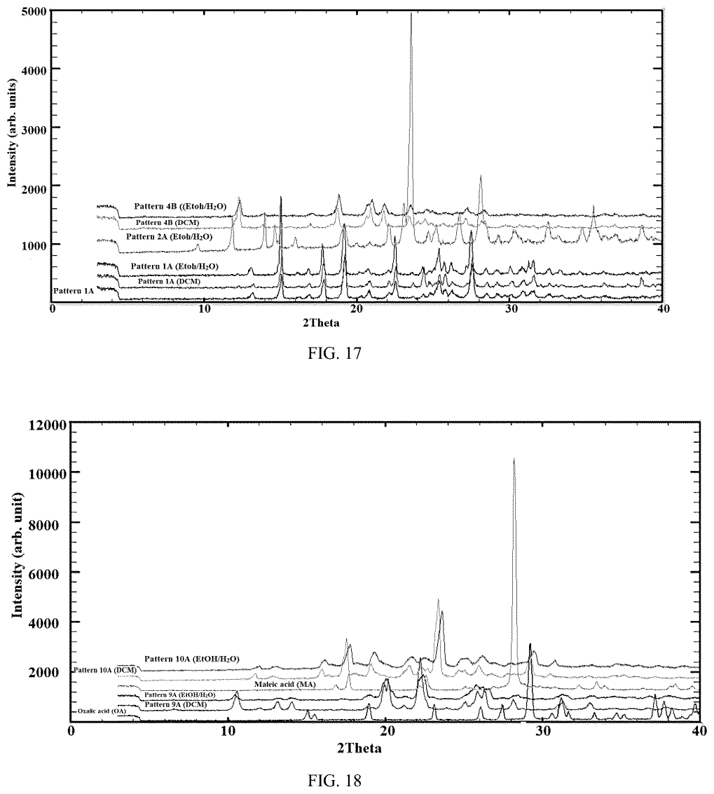

In certain embodiments R/S-5-MAPB H₃PO₄ Pattern 4B is characterized by the XRPD diffractogram in FIG. 17 and/or the DSC graph shown in FIG. 38.
a. In certain embodiments R/S-5-MAPB H₃PO₄ Pattern 4C is characterized by an XRPD pattern with three or more peaks selected from 12.9, 14.5, 16.3, 17.6, 18.1, 21.3, 22.0, 24.7, 25.5, 25.9, 26.6, 27.4, 28.5, 29.3, 30.6, and 35.7+/−0.4° 2theta.
b. In certain embodiments R/S-5-MAPB H₃PO₄ Pattern 4C is characterized by an XRPD pattern with four or more peaks selected from 12.9, 14.5, 16.3, 17.6, 18.1, 21.3, 22.0, 24.7, 25.5, 25.9, 26.6, 27.4, 28.5, 29.3, 30.6, and 35.7+/−0.4° 2theta.
c. In certain embodiments R/S-5-MAPB H₃PO₄ Pattern 4C is characterized by an XRPD pattern with five or more peaks selected from 12.9, 14.5, 16.3, 17.6, 18.1, 21.3, 22.0, 24.7, 25.5, 25.9, 26.6, 27.4, 28.5, 29.3, 30.6, and 35.7+/−0.4° 2theta.
d. In certain embodiments R/S-5-MAPB H₃PO₄ Pattern 4C is characterized by an XRPD pattern with six or more peaks selected from 12.9, 14.5, 16.3, 17.6, 18.1, 21.3, 22.0, 24.7, 25.5, 25.9, 26.6, 27.4, 28.5, 29.3, 30.6, and 35.7+/−0.4° 2theta.
e. In certain embodiments R/S-5-MAPB H₃PO₄ Pattern 4C is characterized by an XRPD pattern with seven or more peaks selected from 12.9, 14.5, 16.3, 17.6, 18.1, 21.3, 22.0, 24.7, 25.5, 25.9, 26.6, 27.4, 28.5, 29.3, 30.6, and 35.7+/−0.4° 2theta.
f. In certain embodiments R/S-5-MAPB H₃PO₄ Pattern 4C is characterized by any one of embodiments a-e wherein the XRPD pattern includes a peak at 22.0+/−0.4° 2theta.
g. In certain embodiments R/S-5-MAPB H₃PO₄ Pattern 4C is characterized by any one of embodiments a-f wherein the XRPD pattern includes a peak at 21.3+/−0.4° 2theta.
h. In certain embodiments R/S-5-MAPB H₃PO₄ Pattern 4C is characterized by any one of embodiments a-g wherein the XRPD pattern includes a peak at 28.5+/−0.4° 2theta.
i. In certain embodiments R/S-5-MAPB H₃PO₄ Pattern 4C is characterized by any one of embodiments a-h wherein the XRPD pattern includes a peak at 27.4+/−0.4° 2theta.
j. In certain embodiments R/S-5-MAPB H₃PO₄ Pattern 4C is characterized by any one of embodiments a-i wherein the XRPD pattern includes a peak at 25.9+/−0.4° 2theta.
k. In certain embodiments R/S-5-MAPB H₃PO₄ Pattern 4C is characterized by any one of embodiments a-j wherein the peaks are within +/−0.3° 2theta.
l. In certain embodiments R/S-5-MAPB H₃PO₄ Pattern 4C is characterized by any one of embodiments a-j wherein the peaks are within +/−0.2° 2theta.
m. In certain embodiments R/S-5-MAPB H₃PO₄ Pattern 4C is characterized by any one of embodiments a-l wherein at least three of the recited peaks have a relative peak intensity of at least 10%.
n. In certain embodiments R/S-5-MAPB H₃PO₄ Pattern 4C is characterized by any one of embodiments a-l wherein at least three of the recited peaks have a relative peak intensity of at least 20%.
o. In certain embodiments R/S-5-MAPB H₃PO₄ Pattern 4C is characterized by any one of embodiments a-l wherein at least four of the recited peaks have a relative peak intensity of at least 10%.
p. In certain embodiments R/S-5-MAPB H₃PO₄ Pattern 4C is characterized by any one of embodiments a-l wherein at least four of the recited peaks have a relative peak intensity of at least 20%.
q. In certain embodiments R/S-5-MAPB H₃PO₄ Pattern 4C is characterized by any one of embodiments a-l wherein at least five of the recited peaks have a relative peak intensity of at least 10%.
r. In certain embodiments R/S-5-MAPB H₃PO₄ Pattern 4C is characterized by any one of embodiments a-l wherein at least six of the recited peaks have a relative peak intensity of at least 20%.

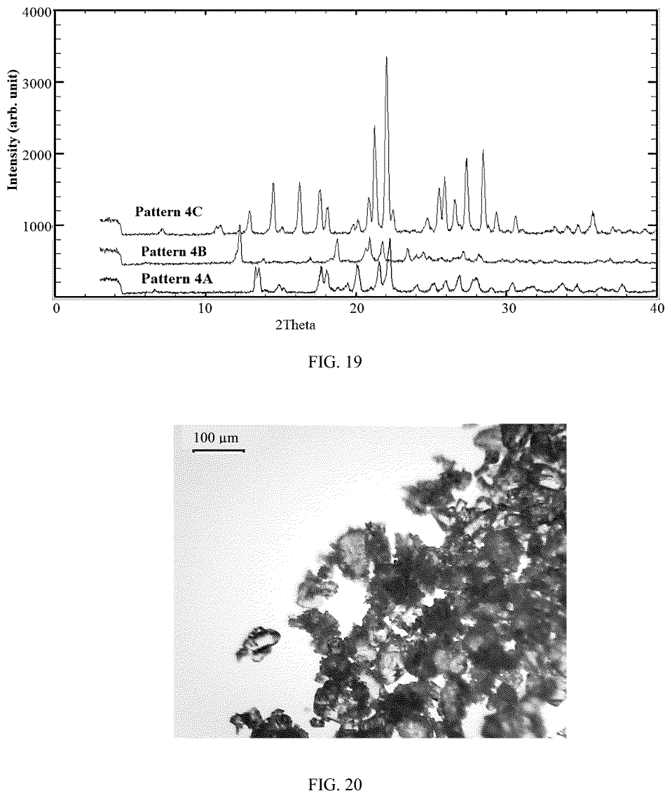

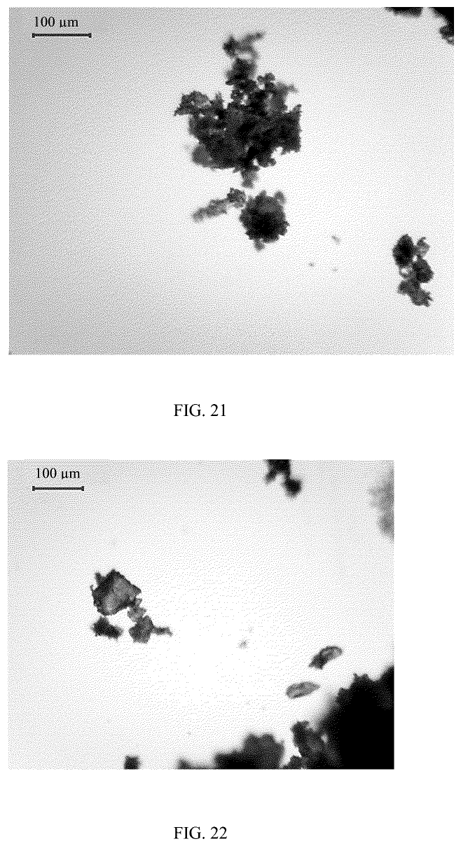

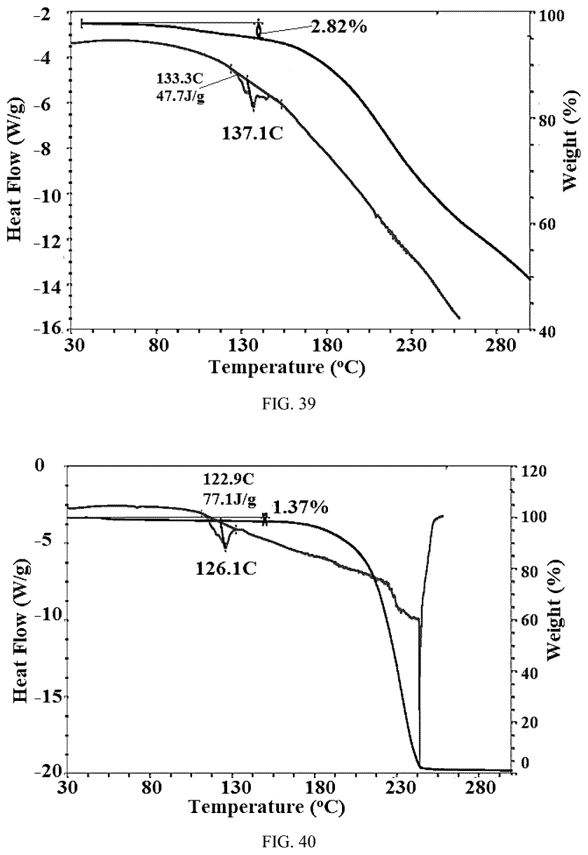

In certain embodiments R/S-5-MAPB H₃PO₄ Pattern 4C is characterized by the XRPD diffractogram in FIG. 19 and/or the DSC graph shown in FIG. 39.

### Oxalic Salt

In certain aspects the invention provides R/S-5-MAPB as an oxalate salt. In certain embodiments the R/S-5-MAPB oxalate salt is a stable morphic form denoted Pattern 9A.
a. In certain embodiments R/S-5-MAPB oxalate Pattern 9A is characterized by an XRPD pattern with three or more peaks selected from 10.6, 13.2, 14.1, 18.9, 19.9, 21.0, 22.2, 22.9, 23.8, 24.7, 25.7, 26.4, 28.2, 31.1, 33.0, 35.3, 36.8, and 37.9+/−0.4° 2theta.
b. In certain embodiments R/S-5-MAPB oxalate Pattern 9A is characterized by an XRPD pattern with four or more peaks selected from 10.6, 13.2, 14.1, 18.9, 19.9, 21.0, 22.2, 22.9, 23.8, 24.7, 25.7, 26.4, 28.2, 31.1, 33.0, 35.3, 36.8, and 37.9+/−0.4° 2theta.
c. In certain embodiments R/S-5-MAPB oxalate Pattern 9A is characterized by an XRPD pattern with five or more peaks selected from 10.6, 13.2, 14.1, 18.9, 19.9, 21.0, 22.2, 22.9, 23.8, 24.7, 25.7, 26.4, 28.2, 31.1, 33.0, 35.3, 36.8, and 37.9+/−0.4° 2theta.
d. In certain embodiments R/S-5-MAPB oxalate Pattern 9A is characterized by an XRPD pattern with six or more peaks selected from 10.6, 13.2, 14.1, 18.9, 19.9, 21.0, 22.2, 22.9, 23.8, 24.7, 25.7, 26.4, 28.2, 31.1, 33.0, 35.3, 36.8, and 37.9+/−0.4° 2theta.
e. In certain embodiments R/S-5-MAPB oxalate Pattern 9A is characterized by an XRPD pattern with seven or more peaks selected from 10.6, 13.2, 14.1, 18.9, 19.9, 21.0, 22.2, 22.9, 23.8, 24.7, 25.7, 26.4, 28.2, 31.1, 33.0, 35.3, 36.8, and 37.9+/−0.4° 2theta.
f. In certain embodiments R/S-5-MAPB oxalate Pattern 9A is characterized by any one of embodiments a-e wherein the XRPD pattern includes a peak at 19.9+/−0.4° 2theta.
g. In certain embodiments R/S-5-MAPB oxalate Pattern 9A is characterized by any one of embodiments a-f wherein the XRPD pattern includes a peak at 22.2+/−0.4° 2theta.
h. In certain embodiments R/S-5-MAPB oxalate Pattern 9A is characterized by any one of embodiments a-g wherein the XRPD pattern includes a peak at 25.7+/−0.4° 2theta.
i. In certain embodiments R/S-5-MAPB oxalate Pattern 9A is characterized by any one of embodiments a-h wherein the XRPD pattern includes a peak at 21.0+/−0.4° 2theta.
j. In certain embodiments R/S-5-MAPB oxalate Pattern 9A is characterized by any one of embodiments a-i wherein the XRPD pattern includes a peak at 26.4+/−0.4° 2theta.
k. In certain embodiments R/S-5-MAPB oxalate Pattern 9A is characterized by any one of embodiments a-j wherein the peaks are within +/−0.3° 2theta.
l. In certain embodiments R/S-5-MAPB oxalate Pattern 9A is characterized by any one of embodiments a-j wherein the peaks are within +/−0.2° 2theta.
m. In certain embodiments R/S-5-MAPB oxalate Pattern 9A is characterized by any one of embodiments a-l wherein at least three of the recited peaks have a relative peak intensity of at least 10%.
n. In certain embodiments R/S-5-MAPB oxalate Pattern 9A is characterized by any one of embodiments a-l wherein at least three of the recited peaks have a relative peak intensity of at least 20%.
o. In certain embodiments R/S-5-MAPB oxalate Pattern 9A is characterized by any one of embodiments a-l wherein at least four of the recited peaks have a relative peak intensity of at least 10%.
p. In certain embodiments R/S-5-MAPB oxalate Pattern 9A is characterized by any one of embodiments a-l wherein at least four of the recited peaks have a relative peak intensity of at least 20%.
q. In certain embodiments R/S-5-MAPB oxalate Pattern 9A is characterized by any one of embodiments a-l wherein at least five of the recited peaks have a relative peak intensity of at least 10%.
r. In certain embodiments R/S-5-MAPB oxalate Pattern 9A is characterized by any one of embodiments a-l wherein at least six of the recited peaks have a relative peak intensity of at least 20%.

In certain embodiments R/S-5-MAPB oxalate Pattern 9A is characterized by the XRPD diffractogram in FIG. 16 and/or the DSC graph shown in FIG. 40.

### Maleic Salt

In certain aspects the invention provides R/S-5-MAPB as a maleic salt. In certain embodiments the R/S-5-MAPB maleic salt is a stable morphic form denoted Pattern 10A.
a. In certain embodiments R/S-5-MAPB maleic Pattern 10A is characterized by an XRPD pattern with three or more peaks selected from 16.1, 17.5, 17.7, 18.7, 19.3, 19.7, 21.7, 22.5, 22.8, 23.4, 23.5, 24.8, 26.1, and 29.4+/−0.4° 2theta.
b. In certain embodiments R/S-5-MAPB maleic Pattern 10A is characterized by an XRPD pattern with four or more peaks selected from 16.1, 17.5, 17.7, 18.7, 19.3, 19.7, 21.7, 22.5, 22.8, 23.4, 23.5, 24.8, 26.1, and 29.4+/−0.4° 2theta.
c. In certain embodiments R/S-5-MAPB maleic Pattern 10A is characterized by an XRPD pattern with five or more peaks selected from 16.1, 17.5, 17.7, 18.7, 19.3, 19.7, 21.7, 22.5, 22.8, 23.4, 23.5, 24.8, 26.1, and 29.4+/−0.4° 2theta.
d. In certain embodiments R/S-5-MAPB maleic Pattern 10A is characterized by an XRPD pattern with six or more peaks selected from 16.1, 17.5, 17.7, 18.7, 19.3, 19.7, 21.7, 22.5, 22.8, 23.4, 23.5, 24.8, 26.1, and 29.4+/−0.4° 2theta.
e. In certain embodiments R/S-5-MAPB maleic Pattern 10A is characterized by an XRPD pattern with seven or more peaks selected from 16.1, 17.5, 17.7, 18.7, 19.3, 19.7, 21.7, 22.5, 22.8, 23.4, 23.5, 24.8, 26.1, and 29.4+/−0.4° 2theta.
f. In certain embodiments R/S-5-MAPB maleic Pattern 10A is characterized by any one of embodiments a-e wherein the XRPD pattern includes a peak at 23.5+/−0.4° 2theta.
g. In certain embodiments R/S-5-MAPB maleic Pattern 10A is characterized by any one of embodiments a-f wherein the XRPD pattern includes a peak at 23.4+/−0.4° 2theta.
h. In certain embodiments R/S-5-MAPB maleic Pattern 10A is characterized by any one of embodiments a-g wherein the XRPD pattern includes a peak at 17.7+/−0.4° 2theta.
i. In certain embodiments R/S-5-MAPB maleic Pattern 10A is characterized by any one of embodiments a-h wherein the XRPD pattern includes a peak at 29.4+/−0.4° 2theta.
j. In certain embodiments R/S-5-MAPB maleic Pattern 10A is characterized by any one of embodiments a-i wherein the XRPD pattern includes a peak at 19.3+/−0.4° 2theta.
k. In certain embodiments R/S-5-MAPB maleic Pattern 10A is characterized by any one of embodiments a-j wherein the peaks are within +/−0.3° 2theta.
l. In certain embodiments R/S-5-MAPB maleic Pattern 10A is characterized by any one of embodiments a-j wherein the peaks are within +/−0.2° 2theta.
m. In certain embodiments R/S-5-MAPB maleic Pattern 10A is characterized by any one of embodiments a-l wherein at least three of the recited peaks have a relative peak intensity of at least 10%.
n. In certain embodiments R/S-5-MAPB maleic Pattern 10A is characterized by any one of embodiments a-l wherein at least three of the recited peaks have a relative peak intensity of at least 20%.
o. In certain embodiments R/S-5-MAPB maleic Pattern 10A is characterized by any one of embodiments a-l wherein at least four of the recited peaks have a relative peak intensity of at least 10%.
p. In certain embodiments R/S-5-MAPB maleic Pattern 10A is characterized by any one of embodiments a-l wherein at least four of the recited peaks have a relative peak intensity of at least 20% q. In certain embodiments R/S-5-MAPB maleic Pattern 10A is characterized by any one of embodiments a-l wherein at least five of the recited peaks have a relative peak intensity of at least 10%.
r. In certain embodiments R/S-5-MAPB maleic Pattern 10A is characterized by any one of embodiments a-l wherein at least six of the recited peaks have a relative peak intensity of at least 20%.

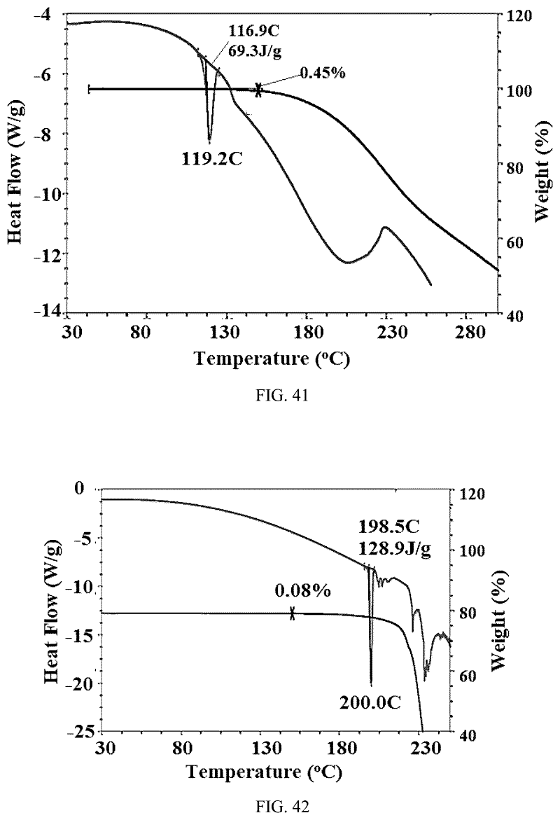

In certain embodiments R/S-5-MAPB maleic Pattern 10A is characterized by the XRPD diffractogram in FIG. 17 and/or the DSC graph shown in FIG. 41.

### Morphic Forms of S-5-MAPB

### HCl Salt

In certain aspects the invention provides S-5-MAPB as an HCl salt for therapeutic uses. In certain embodiments the S-5-MAPB HCl salt is a stable morphic form denoted Pattern 1A.
a. In certain embodiments S-5-MAPB HCl Pattern 1A is characterized by an XRPD pattern with three or more peaks selected from 6.7, 12.7, 13.4, 15.8, 19.0, 19.6, 21.2, 24.7, 25.1, 26.1, 26.8, 28.1, 29.0, 30.4, 31.1, and 39.7+/−0.4° 2theta.
b. In certain embodiments S-5-MAPB HCl Pattern 1A is characterized by an XRPD pattern with four or more peaks selected from 6.7, 12.7, 13.4, 15.8, 19.0, 19.6, 21.2, 24.7, 25.1, 26.1, 26.8, 28.1, 29.0, 30.4, 31.1, and 39.7+/−0.4° 2theta.
c. In certain embodiments S-5-MAPB HCl Pattern 1A is characterized by an XRPD pattern with five or more peaks selected from 6.7, 12.7, 13.4, 15.8, 19.0, 19.6, 21.2, 24.7, 25.1, 26.1, 26.8, 28.1, 29.0, 30.4, 31.1, and 39.7+/−0.4° 2theta.
d. In certain embodiments S-5-MAPB HCl Pattern 1A is characterized by an XRPD pattern with six or more peaks selected from 6.7, 12.7, 13.4, 15.8, 19.0, 19.6, 21.2, 24.7, 25.1, 26.1, 26.8, 28.1, 29.0, 30.4, 31.1, and 39.7+/−0.4° 2theta.
e. In certain embodiments S-5-MAPB HCl Pattern 1A is characterized by an XRPD pattern with seven or more peaks selected from 6.7, 12.7, 13.4, 15.8, 19.0, 19.6, 21.2, 24.7, 25.1, 26.1, 26.8, 28.1, 29.0, 30.4, 31.1, and 39.7+/−0.4° 2theta.
f. In certain embodiments S-5-MAPB HCl Pattern 1A is characterized by any one of embodiments a-e wherein the XRPD pattern includes a peak at 26.8+/−0.4° 2theta.
g. In certain embodiments S-5-MAPB HCl Pattern 1A is characterized by any one of embodiments a-f wherein the XRPD pattern includes a peak at 19.0+/−0.4° 2theta.
h. In certain embodiments S-5-MAPB HCl Pattern 1A is characterized by any one of embodiments a-g wherein the XRPD pattern includes a peak at 24.7+/−0.4° 2theta.
i. In certain embodiments S-5-MAPB HCl Pattern 1A is characterized by any one of embodiments a-h wherein the XRPD pattern includes a peak at 25.1+/−0.4° 2theta.
j. In certain embodiments S-5-MAPB HCl Pattern 1A is characterized by any one of embodiments a-i wherein the XRPD pattern includes a peak at 13.4+/−0.4° 2theta.
k. In certain embodiments S-5-MAPB HCl Pattern 1A is characterized by any one of embodiments a-j wherein the peaks are within +/−0.3° 2theta.
l. In certain embodiments S-5-MAPB HCl Pattern 1A is characterized by any one of embodiments a-j wherein the peaks are within +/−0.2° 2theta.
m. In certain embodiments S-5-MAPB HCl Pattern 1A is characterized by any one of embodiments a-l wherein at least three of the recited peaks have a relative peak intensity of at least 5%.
n. In certain embodiments S-5-MAPB HCl Pattern 1A is characterized by any one of embodiments a-l wherein at least three of the recited peaks have a relative peak intensity of at least 10%.
o. In certain embodiments S-5-MAPB HCl Pattern 1A is characterized by any one of embodiments a-l wherein at least four of the recited peaks have a relative peak intensity of at least 5%.
p. In certain embodiments S-5-MAPB HCl Pattern 1A is characterized by any one of embodiments a-l wherein at least four of the recited peaks have a relative peak intensity of at least 10%.
q. In certain embodiments S-5-MAPB HCl Pattern 1A is characterized by any one of embodiments a-l wherein at least five of the recited peaks have a relative peak intensity of at least 5%.
r. In certain embodiments S-5-MAPB HCl Pattern 1A is characterized by any one of embodiments a-l wherein at least six of the recited peaks have a relative peak intensity of at least 5%.

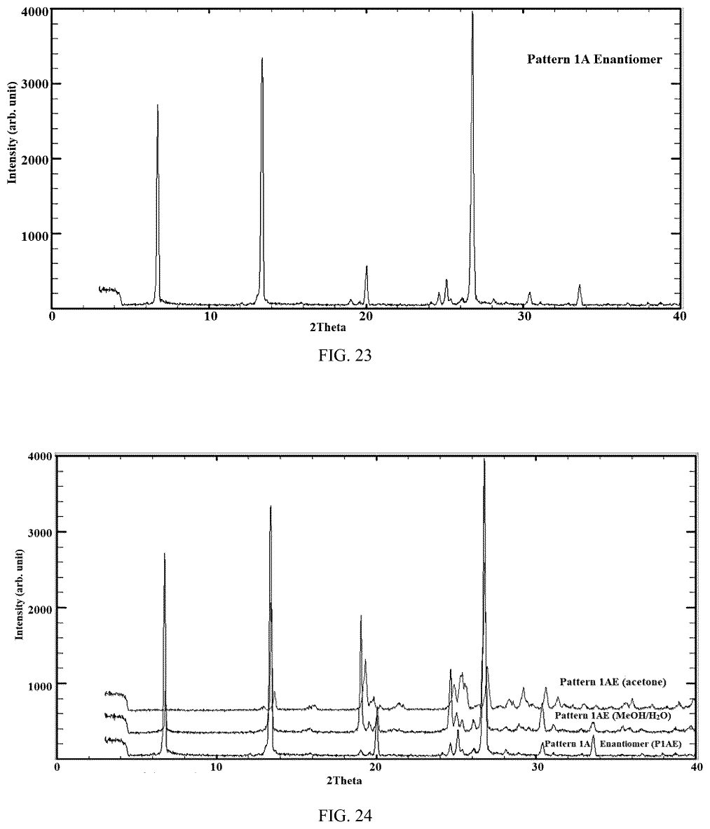

In certain embodiments S-5-MAPB HCl Pattern 1A is characterized by the XRPD diffractogram in FIG. 23 and/or the DSC graph shown in FIG. 42.

### HBr Salt

In certain aspects the invention provides S-5-MAPB as an HBr salt. In certain embodiments the S-5-MAPB HBr salt is a stable morphic form denoted Pattern 2A.
a. In certain embodiments S-5-MAPB HBr Pattern 2A is characterized by an XRPD pattern with three or more peaks selected from 13.3, 19.0, 19.8, 20.3, 24.6, 26.0, 26.4, 27.2, 28.6, 30.1, 30.9, 33.1, and 35.3+/−0.4° 2theta.
b. In certain embodiments S-5-MAPB HBr Pattern 2A is characterized by an XRPD pattern with four or more peaks selected from 13.3, 19.0, 19.8, 20.3, 24.6, 26.0, 26.4, 27.2, 28.6, 30.1, 30.9, 33.1, and 35.3+/−0.4° 2theta.
c. In certain embodiments S-5-MAPB HBr Pattern 2A is characterized by an XRPD pattern with five or more peaks selected from 13.3, 19.0, 19.8, 20.3, 24.6, 26.0, 26.4, 27.2, 28.6, 30.1, 30.9, 33.1, and 35.3+/−0.4° 2theta.
d. In certain embodiments S-5-MAPB HBr Pattern 2A is characterized by an XRPD pattern with six or more peaks selected from 13.3, 19.0, 19.8, 20.3, 24.6, 26.0, 26.4, 27.2, 28.6, 30.1, 30.9, 33.1, and 35.3+/−0.4° 2theta.
e. In certain embodiments S-5-MAPB HBr Pattern 2A is characterized by an XRPD pattern with seven or more peaks selected from 13.3, 19.0, 19.8, 20.3, 24.6, 26.0, 26.4, 27.2, 28.6, 30.1, 30.9, 33.1, and 35.3+/−0.4° 2theta.
f. In certain embodiments S-5-MAPB HBr Pattern 2A is characterized by any one of embodiments a-e wherein the XRPD pattern includes a peak at 26.4+/−0.4° 2theta.
g. In certain embodiments S-5-MAPB HBr Pattern 2A is characterized by any one of embodiments a-f wherein the XRPD pattern includes a peak at 26.0+/−0.4° 2theta.
h. In certain embodiments S-5-MAPB HBr Pattern 2A is characterized by any one of embodiments a-g wherein the XRPD pattern includes a peak at 13.3+/−0.4° 2theta.
i. In certain embodiments S-5-MAPB HBr Pattern 2A is characterized by any one of embodiments a-h wherein the XRPD pattern includes a peak at 24.6+/−0.4° 2theta.
j. In certain embodiments S-5-MAPB HBr Pattern 2A is characterized by any one of embodiments a-i wherein the XRPD pattern includes a peak at 33.1+/−0.4° 2theta.
k. In certain embodiments S-5-MAPB HBr Pattern 2A is characterized by any one of embodiments a-j wherein the peaks are within +/−0.3° 2theta.
l. In certain embodiments S-5-MAPB HBr Pattern 2A is characterized by any one of embodiments a-j wherein the peaks are within +/−0.2° 2theta.
m. In certain embodiments S-5-MAPB HBr Pattern 2A is characterized by any one of embodiments a-l wherein at least three of the recited peaks have a relative peak intensity of at least 10%.
n. In certain embodiments S-5-MAPB HBr Pattern 2A is characterized by any one of embodiments a-l wherein at least three of the recited peaks have a relative peak intensity of at least 20%.
o. In certain embodiments S-5-MAPB HBr Pattern 2A is characterized by any one of embodiments a-l wherein at least four of the recited peaks have a relative peak intensity of at least 10%.
p. In certain embodiments S-5-MAPB HBr Pattern 2A is characterized by any one of embodiments a-l wherein at least four of the recited peaks have a relative peak intensity of at least 20%.
q. In certain embodiments S-5-MAPB HBr Pattern 2A is characterized by any one of embodiments a-l wherein at least five of the recited peaks have a relative peak intensity of at least 10%.
r. In certain embodiments S-5-MAPB HBr Pattern 2A is characterized by any one of embodiments a-l wherein at least six of the recited peaks have a relative peak intensity of at least 20%.

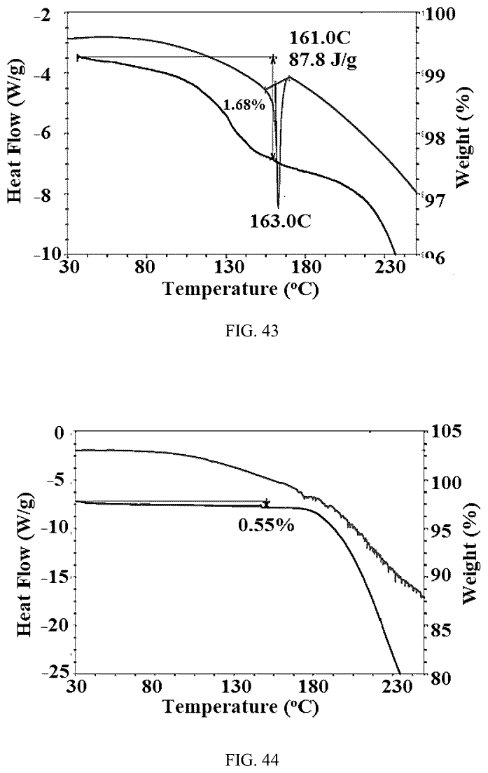

In certain embodiments S-5-MAPB HBr Pattern 2A is characterized by the XRPD diffractogram in FIG. 28 and/or the DSC graph shown in FIG. 43.

H₃PO₄ Salt

In certain aspects the invention provides S-5-MAPB as an H₃PO₄ salt. In certain embodiments the S-5-MAPB H₃PO₄ salt is a stable morphic form denoted Pattern 4A.
a. In certain embodiments S-5-MAPB H₃PO₄ Pattern 4A is characterized by an XRPD pattern with three or more peaks selected from 13.3, 16.4, 17.5, 19.2, 20.0, 21.9, 22.6, 23.9, 24.9, 26.1, and 27.3+/−0.4° 2theta.
b. In certain embodiments S-5-MAPB H₃PO₄ Pattern 4A is characterized by an XRPD pattern with four or more peaks selected from 13.3, 16.4, 17.5, 19.2, 20.0, 21.9, 22.6, 23.9, 24.9, 26.1, and 27.3+/−0.4° 2theta.
c. In certain embodiments S-5-MAPB H₃PO₄ Pattern 4A is characterized by an XRPD pattern with five or more peaks selected from 13.3, 16.4, 17.5, 19.2, 20.0, 21.9, 22.6, 23.9, 24.9, 26.1, and 27.3+/−0.4° 2theta.
d. In certain embodiments S-5-MAPB H₃PO₄ Pattern 4A is characterized by an XRPD pattern with six or more peaks selected from 13.3, 16.4, 17.5, 19.2, 20.0, 21.9, 22.6, 23.9, 24.9, 26.1, and 27.3+/−0.4° 2theta.
e. In certain embodiments S-5-MAPB H₃PO₄ Pattern 4A is characterized by an XRPD pattern with seven or more peaks selected from 13.3, 16.4, 17.5, 19.2, 20.0, 21.9, 22.6, 23.9, 24.9, 26.1, and 27.3+/−0.4° 2theta.
f. In certain embodiments S-5-MAPB H₃PO₄ Pattern 4A is characterized by any one of embodiments a-e wherein the XRPD pattern includes a peak at 13.3+/−0.4° 2theta.
g. In certain embodiments S-5-MAPB H₃PO₄ Pattern 4A is characterized by any one of embodiments a-f wherein the XRPD pattern includes a peak at 21.9+/−0.4° 2theta.
h. In certain embodiments S-5-MAPB H₃PO₄ Pattern 4A is characterized by any one of embodiments a-g wherein the XRPD pattern includes a peak at 17.5+/−0.4° 2theta.
i. In certain embodiments S-5-MAPB H₃PO₄ Pattern 4A is characterized by any one of embodiments a-h wherein the XRPD pattern includes a peak at 27.3+/−0.4° 2theta.
j. In certain embodiments S-5-MAPB H₃PO₄ Pattern 4A is characterized by any one of embodiments a-i wherein the XRPD pattern includes a peak at 26.1+/−0.4° 2theta.
k. In certain embodiments S-5-MAPB H₃PO₄ Pattern 4A is characterized by any one of embodiments a-j wherein the peaks are within +/−0.3° 2theta.
l. In certain embodiments S-5-MAPB H₃PO₄ Pattern 4A is characterized by any one of embodiments a-j wherein the peaks are within +/−0.2° 2theta.
m. In certain embodiments S-5-MAPB H₃PO₄ Pattern 4A is characterized by any one of embodiments a-l wherein at least three of the recited peaks have a relative peak intensity of at least 10%.
n. In certain embodiments S-5-MAPB H₃PO₄ Pattern 4A is characterized by any one of embodiments a-l wherein at least three of the recited peaks have a relative peak intensity of at least 20%.
o. In certain embodiments S-5-MAPB H₃PO₄ Pattern 4A is characterized by any one of embodiments a-l wherein at least four of the recited peaks have a relative peak intensity of at least 10%.
p. In certain embodiments S-5-MAPB H₃PO₄ Pattern 4A is characterized by any one of embodiments a-l wherein at least four of the recited peaks have a relative peak intensity of at least 20%.
q. In certain embodiments S-5-MAPB H₃PO₄ Pattern 4A is characterized by any one of embodiments a-l wherein at least five of the recited peaks have a relative peak intensity of at least 10%.
r. In certain embodiments S-5-MAPB H₃PO₄ Pattern 4A is characterized by any one of embodiments a-l wherein at least six of the recited peaks have a relative peak intensity of at least 20%.

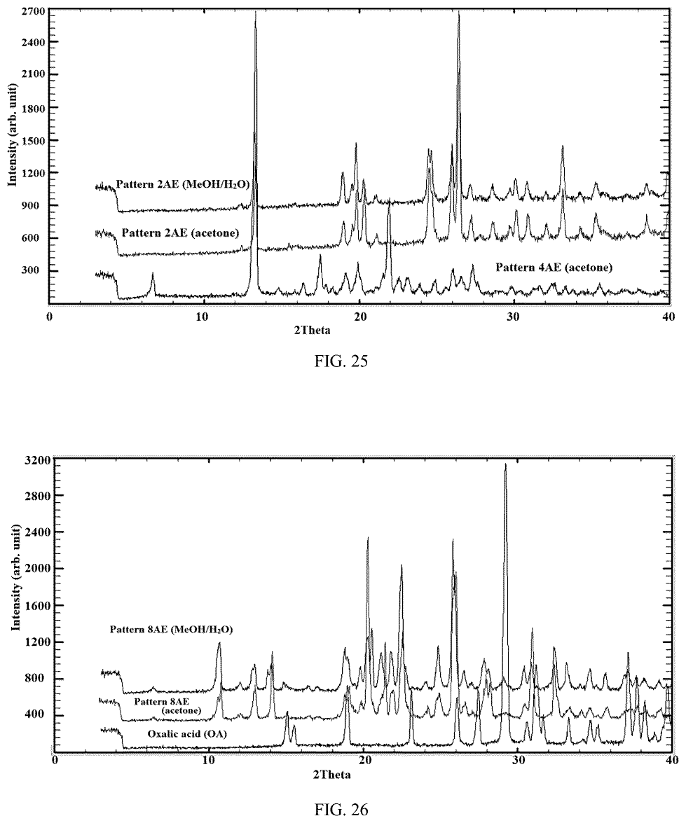

In certain embodiments S-5-MAPB H₃PO₄ Pattern 4A is characterized by the XRPD diffractogram in FIG. 25 and/or the DSC graph shown in FIG. 44

### Oxalic Salt

In certain aspects the invention provides S-5-MAPB as an oxalate salt. In certain embodiments the S-5-MAPB oxalate salt is a stable morphic form denoted Pattern 8A.
a. In certain embodiments S-5-MAPB oxalate Pattern 8A is characterized by an XRPD pattern with three or more peaks selected from 10.6, 12.9, 14.0, 18.9, 20.3, 20.6, 21.2, 21.8, 22.5, 24.8, 25.9, 26.5, 27.8, 30.4, 30.9, 32.4, 33.2, 34.7, 35.7, and 37.1+/−0.4° 2theta.
b. In certain embodiments S-5-MAPB oxalate Pattern 8A is characterized by an XRPD pattern with four or more peaks selected from 10.6, 12.9, 14.0, 18.9, 20.3, 20.6, 21.2, 21.8, 22.5, 24.8, 25.9, 26.5, 27.8, 30.4, 30.9, 32.4, 33.2, 34.7, 35.7, and 37.1+/−0.4° 2theta.
c. In certain embodiments S-5-MAPB oxalate Pattern 8A is characterized by an XRPD pattern with five or more peaks selected from 10.6, 12.9, 14.0, 18.9, 20.3, 20.6, 21.2, 21.8, 22.5, 24.8, 25.9, 26.5, 27.8, 30.4, 30.9, 32.4, 33.2, 34.7, 35.7, and 37.1+/−0.4° 2theta.
d. In certain embodiments S-5-MAPB oxalate Pattern 8A is characterized by an XRPD pattern with six or more peaks selected from 10.6, 12.9, 14.0, 18.9, 20.3, 20.6, 21.2, 21.8, 22.5, 24.8, 25.9, 26.5, 27.8, 30.4, 30.9, 32.4, 33.2, 34.7, 35.7, and 37.1+/−0.4° 2theta.
e. In certain embodiments S-5-MAPB oxalate Pattern 8A is characterized by an XRPD pattern with seven or more peaks selected from 10.6, 12.9, 14.0, 18.9, 20.3, 20.6, 21.2, 21.8, 22.5, 24.8, 25.9, 26.5, 27.8, 30.4, 30.9, 32.4, 33.2, 34.7, 35.7, and 37.1+/−0.4° 2theta.
f. In certain embodiments S-5-MAPB oxalate Pattern 8A is characterized by any one of embodiments a-e wherein the XRPD pattern includes a peak at 22.5+/−0.4° 2theta.
g. In certain embodiments S-5-MAPB oxalate Pattern 8A is characterized by any one of embodiments a-f wherein the XRPD pattern includes a peak at 25.9+/−0.4° 2theta.
h. In certain embodiments S-5-MAPB oxalate Pattern 8A is characterized by any one of embodiments a-g wherein the XRPD pattern includes a peak at 20.6+/−0.4° 2theta.
i. In certain embodiments S-5-MAPB oxalate Pattern 8A is characterized by any one of embodiments a-h wherein the XRPD pattern includes a peak at 20.3+/−0.4° 2theta.
j. In certain embodiments S-5-MAPB oxalate Pattern 8A is characterized by any one of embodiments a-i wherein the XRPD pattern includes a peak at 10.6+/−0.4° 2theta.
k. In certain embodiments S-5-MAPB oxalate Pattern 8A is characterized by any one of embodiments a-j wherein the peaks are within +/−0.3° 2theta.
l. In certain embodiments S-5-MAPB oxalate Pattern 8A is characterized by any one of embodiments a-j wherein the peaks are within +/−0.2° 2theta.
m. In certain embodiments S-5-MAPB oxalate Pattern 8A is characterized by any one of embodiments a-l wherein at least three of the recited peaks have a relative peak intensity of at least 10%.
n. In certain embodiments S-5-MAPB oxalate Pattern 8A is characterized by any one of embodiments a-l wherein at least three of the recited peaks have a relative peak intensity of at least 20%.
o. In certain embodiments S-5-MAPB oxalate Pattern 8A is characterized by any one of embodiments a-l wherein at least four of the recited peaks have a relative peak intensity of at least 10%.
p. In certain embodiments S-5-MAPB oxalate Pattern 8A is characterized by any one of embodiments a-l wherein at least four of the recited peaks have a relative peak intensity of at least 20%.
q. In certain embodiments S-5-MAPB oxalate Pattern 8A is characterized by any one of embodiments a-l wherein at least five of the recited peaks have a relative peak intensity of at least 10%.
r. In certain embodiments S-5-MAPB oxalate Pattern 8A is characterized by any one of embodiments a-l wherein at least six of the recited peaks have a relative peak intensity of at least 20%.

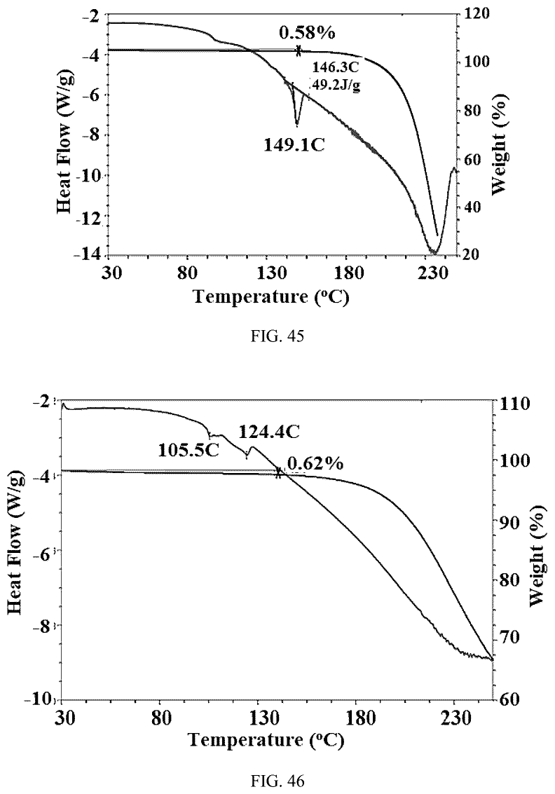

In certain embodiments S-5-MAPB oxalate Pattern 8A is characterized by the XRPD diffractogram in FIG. 26 and/or the DSC graph shown in FIG. 45.

In certain aspects the invention provides S-5-MAPB as a fumaric salt. In certain embodiments the S-5-MAPB fumaric salt is a stable morphic form denoted Pattern 10A.
a. In certain embodiments S-5-MAPB fumaric Pattern 10A is characterized by an XRPD pattern with three or more peaks selected from 8.5, 16.0, 17.6, 18.1, 20.1, 20.9, 21.7, 23.1, 23.6, 24.0, 25.2, 26.2, 28.5, 29.5, 30.4, and 30.7+/−0.4° 2theta.
b. In certain embodiments S-5-MAPB fumaric Pattern 10A is characterized by an XRPD pattern with four or more peaks selected from 8.5, 16.0, 17.6, 18.1, 20.1, 20.9, 21.7, 23.1, 23.6, 24.0, 25.2, 26.2, 28.5, 29.5, 30.4, and 30.7+/−0.4° 2theta.
c. In certain embodiments S-5-MAPB fumaric Pattern 10A is characterized by an XRPD pattern with five or more peaks selected from 8.5, 16.0, 17.6, 18.1, 20.1, 20.9, 21.7, 23.1, 23.6, 24.0, 25.2, 26.2, 28.5, 29.5, 30.4, and 30.7+/−0.4° 2theta.
d. In certain embodiments S-5-MAPB fumaric Pattern 10A is characterized by an XRPD pattern with six or more peaks selected from 8.5, 16.0, 17.6, 18.1, 20.1, 20.9, 21.7, 23.1, 23.6, 24.0, 25.2, 26.2, 28.5, 29.5, 30.4, and 30.7+/−0.4° 2theta.
e. In certain embodiments S-5-MAPB fumaric Pattern 10A is characterized by an XRPD pattern with seven or more peaks selected from 8.5, 16.0, 17.6, 18.1, 20.1, 20.9, 21.7, 23.1, 23.6, 24.0, 25.2, 26.2, 28.5, 29.5, 30.4, and 30.7+/−0.4° 2theta.
f. In certain embodiments S-5-MAPB fumaric Pattern 10A is characterized by any one of embodiments a-e wherein the XRPD pattern includes a peak at 23.6+/−0.4° 2theta.
g. In certain embodiments S-5-MAPB fumaric Pattern 10A is characterized by any one of embodiments a-f wherein the XRPD pattern includes a peak at 18.1+/−0.4° 2theta.
h. In certain embodiments S-5-MAPB fumaric Pattern 10A is characterized by any one of embodiments a-g wherein the XRPD pattern includes a peak at 17.6+/−0.4° 2theta.
i. In certain embodiments S-5-MAPB fumaric Pattern 10A is characterized by any one of embodiments a-h wherein the XRPD pattern includes a peak at 25.2+/−0.4° 2theta.
j. In certain embodiments S-5-MAPB fumaric Pattern 10A is characterized by any one of embodiments a-i wherein the XRPD pattern includes a peak at 23.1+/−0.4° 2theta.
k. In certain embodiments S-5-MAPB fumaric Pattern 10A is characterized by any one of embodiments a-j wherein the peaks are within +/−0.3° 2theta.
l. In certain embodiments S-5-MAPB fumaric Pattern 10A is characterized by any one of embodiments a-j wherein the peaks are within +/−0.2° 2theta.
m. In certain embodiments S-5-MAPB fumaric Pattern 10A is characterized by any one of embodiments a-l wherein at least three of the recited peaks have a relative peak intensity of at least 10%.
n. In certain embodiments S-5-MAPB fumaric Pattern 10A is characterized by any one of embodiments a-l wherein at least three of the recited peaks have a relative peak intensity of at least 20%.
o. In certain embodiments S-5-MAPB fumaric Pattern 10A is characterized by any one of embodiments a-l wherein at least four of the recited peaks have a relative peak intensity of at least 10%.
p. In certain embodiments S-5-MAPB fumaric Pattern 10A is characterized by any one of embodiments a-l wherein at least four of the recited peaks have a relative peak intensity of at least 20%.
q. In certain embodiments S-5-MAPB fumaric Pattern 10A is characterized by any one of embodiments a-l wherein at least five of the recited peaks have a relative peak intensity of at least 10%.
r. In certain embodiments S-5-MAPB fumaric Pattern 10A is characterized by any one of embodiments a-l wherein at least six of the recited peaks have a relative peak intensity of at least 20%.

In certain embodiments S-5-MAPB fumaric Pattern 10A is characterized by the XRPD diffractogram in FIG. 30 and/or the DSC graph shown in FIG. 46.

### Morphic Forms of S-BK-5-MAPB

### HCl Salt

### Pattern 1A

In certain aspects the invention provides S-BK-5-MAPB as HCl salt. In certain embodiments the S-BK-5-MAPB salt is a stable morphic form denoted Pattern 1A.
a. In certain embodiments S-BK-5-MAPB Pattern 1A is characterized by an XRPD pattern with three or more peaks selected from 5.7, 11.2, 13.2, 14.5, 15.3, 16.8, 17.5, 19.0, 19.8, 20.0, 20.4, 20.6, 21.7, 21.9, 22.4, 24.0, 24.7, 25.0, 27.2, 27.9, 28.2, 28.8, 28.9, 30.2, 30.6, 33.9, and 36.0+/−0.4° 2theta.
b. In certain embodiments S-BK-5-MAPB Pattern 1A is characterized by an XRPD pattern with four or more peaks selected from 5.7, 11.2, 13.2, 14.5, 15.3, 16.8, 17.5, 19.0, 19.8, 20.0, 20.4, 20.6, 21.7, 21.9, 22.4, 24.0, 24.7, 25.0, 27.2, 27.9, 28.2, 28.8, 28.9, 30.2, 30.6, 33.9, and 36.0+/−0.4° 2theta.
c. In certain embodiments S-BK-5-MAPB Pattern 1A is characterized by an XRPD pattern with five or more peaks selected from 5.7, 11.2, 13.2, 14.5, 15.3, 16.8, 17.5, 19.0, 19.8, 20.0, 20.4, 20.6, 21.7, 21.9, 22.4, 24.0, 24.7, 25.0, 27.2, 27.9, 28.2, 28.8, 28.9, 30.2, 30.6, 33.9, and 36.0+/−0.4° 2theta.
d. In certain embodiments S-BK-5-MAPB Pattern 1A is characterized by an XRPD pattern with six or more peaks selected from 5.7, 11.2, 13.2, 14.5, 15.3, 16.8, 17.5, 19.0, 19.8, 20.0, 20.4, 20.6, 21.7, 21.9, 22.4, 24.0, 24.7, 25.0, 27.2, 27.9, 28.2, 28.8, 28.9, 30.2, 30.6, 33.9, and 36.0+/−0.4° 2theta.
e. In certain embodiments S-BK-5-MAPB Pattern 1A is characterized by an XRPD pattern with seven or more peaks selected from 5.7, 11.2, 13.2, 14.5, 15.3, 16.8, 17.5, 19.0, 19.8, 20.0, 20.4, 20.6, 21.7, 21.9, 22.4, 24.0, 24.7, 25.0, 27.2, 27.9, 28.2, 28.8, 28.9, 30.2, 30.6, 33.9, and 36.0+/−0.4° 2theta.
f. In certain embodiments S-BK-5-MAPB Pattern 1A is characterized by any one of embodiments a-e wherein the XRPD pattern includes a peak at 5.7+/−0.4° 2theta.
g. In certain embodiments S-BK-5-MAPB Pattern 1A is characterized by any one of embodiments a-f wherein the XRPD pattern includes a peak at 14.5+/−0.4° 2theta.
h. In certain embodiments S-BK-5-MAPB Pattern 1A is characterized by any one of embodiments a-g wherein the XRPD pattern includes a peak at 15.3+/−0.4° 2theta.
i. In certain embodiments S-BK-5-MAPB Pattern 1A is characterized by any one of embodiments a-h wherein the XRPD pattern includes a peak at 19.0+/−0.4° 2theta.
j. In certain embodiments S-BK-5-MAPB Pattern 1A is characterized by any one of embodiments a-i wherein the XRPD pattern includes a peak at 19.8+/−0.4° 2theta.
k. In certain embodiments S-BK-5-MAPB Pattern 1A is characterized by any one of embodiments a-i wherein the XRPD pattern includes a peak at 21.7+/−0.4° 2theta.
l. In certain embodiments S-BK-5-MAPB Pattern 1A is characterized by any one of embodiments a-i wherein the XRPD pattern includes a peak at 22.4+/−0.4° 2theta.
m. In certain embodiments S-BK-5-MAPB Pattern 1A is characterized by any one of embodiments a-i wherein the XRPD pattern includes a peak at 24.7+/−0.4°2theta.
n. In certain embodiments S-BK-5-MAPB Pattern 1A is characterized by any one of embodiments a-i wherein the XRPD pattern includes a peak at 28.8+/−0.4°2theta.
o. In certain embodiments S-BK-5-MAPB Pattern 1A is characterized by any one of embodiments a-i wherein the XRPD pattern includes a peak at 30.6+/−0.4° 2theta.
p. In certain embodiments S-BK-5-MAPB Pattern 1A is characterized by any one of embodiments a-o wherein the peaks are within +/−0.3°2theta.
q. In certain embodiments S-BK-5-MAPB Pattern 1A is characterized by any one of embodiments a-o wherein the peaks are within +/−0.2°2theta.
r. In certain embodiments S-BK-5-MAPB Pattern 1A is characterized by any one of embodiments a-q wherein at least three of the recited peaks have a relative peak intensity of at least 10%.
s. In certain embodiments S-BK-5-MAPB Pattern 1A is characterized by any one of embodiments a-q wherein at least three of the recited peaks have a relative peak intensity of at least 20%.
t. In certain embodiments S-BK-5-MAPB Pattern 1A is characterized by any one of embodiments a-q wherein at least four of the recited peaks have a relative peak intensity of at least 10%.
u. In certain embodiments S-BK-5-MAPB Pattern 1A is characterized by any one of embodiments a-q wherein at least four of the recited peaks have a relative peak intensity of at least 20%.
v. In certain embodiments S-BK-5-MAPB Pattern 1A is characterized by any one of embodiments a-q wherein at least five of the recited peaks have a relative peak intensity of at least 10%.
w. In certain embodiments S-BK-5-MAPB Pattern 1A is characterized by any one of embodiments a-q wherein at least six of the recited peaks have a relative peak intensity of at least 20%.

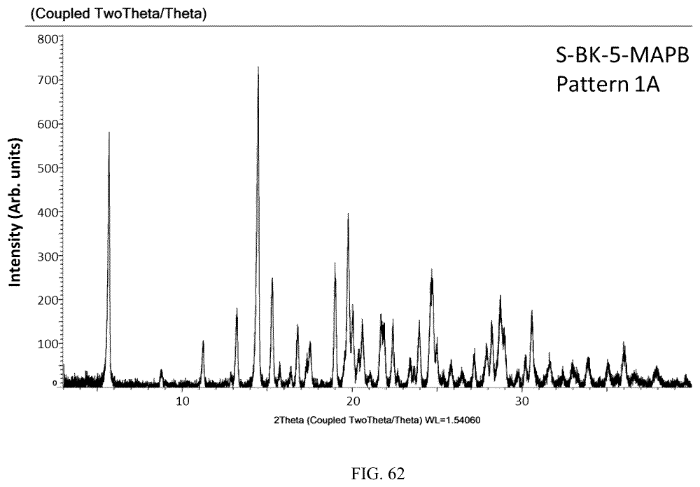

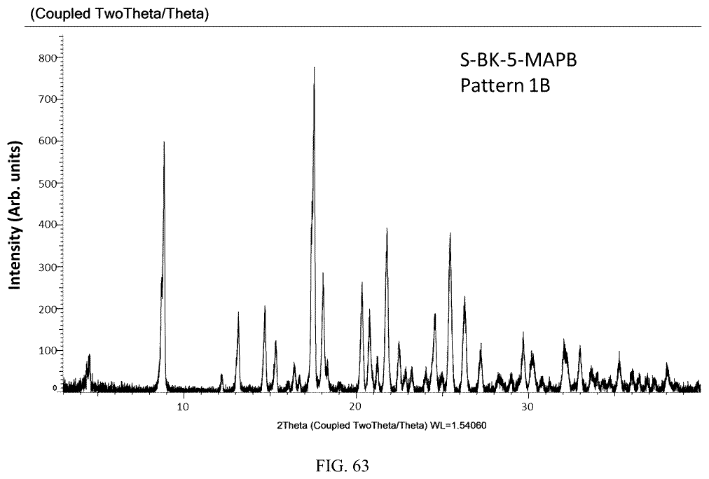

In certain embodiments S-BK-5-MAPB Pattern 1A is characterized by the XRPD diffractogram in FIG. 62.

### Pattern 1B

In certain aspects the invention provides S-BK-5-MAPB as HCl salt. In certain embodiments the S-BK-5-MAPB salt is a stable morphic form denoted Pattern 1B.
a. In certain embodiments S-BK-5-MAPB Pattern 1B is characterized by an XRPD pattern with three or more peaks selected from 4.5, 8.7, 8.9, 13.2, 14.7, 15.3, 17.4, 17.5, 18.1, 20.3, 20.8, 21.2, 21.8, 22.5, 24.6, 25.4, 26.3, 27.2, 29.7, 30.2, 32.1, and 33.0+/−0.4°2theta.
b. In certain embodiments S-BK-5-MAPB Pattern 1B is characterized by an XRPD pattern with four or more peaks selected from 4.5, 8.7, 8.9, 13.2, 14.7, 15.3, 17.4, 17.5, 18.1, 20.3, 20.8, 21.2, 21.8, 22.5, 24.6, 25.4, 26.3, 27.2, 29.7, 30.2, 32.1, and 33.0+/−0.4°2theta.
c. In certain embodiments S-BK-5-MAPB Pattern 1B is characterized by an XRPD pattern with five or more peaks selected from 4.5, 8.7, 8.9, 13.2, 14.7, 15.3, 17.4, 17.5, 18.1, 20.3, 20.8, 21.2, 21.8, 22.5, 24.6, 25.4, 26.3, 27.2, 29.7, 30.2, 32.1, and 33.0+/−0.4°2theta.
d. In certain embodiments S-BK-5-MAPB Pattern 1B is characterized by an XRPD pattern with six or more peaks selected from 4.5, 8.7, 8.9, 13.2, 14.7, 15.3, 17.4, 17.5, 18.1, 20.3, 20.8, 21.2, 21.8, 22.5, 24.6, 25.4, 26.3, 27.2, 29.7, 30.2, 32.1, and 33.0+/−0.4°2theta.
e. In certain embodiments S-BK-5-MAPB Pattern 1B is characterized by an XRPD pattern with seven or more peaks selected from 4.5, 8.7, 8.9, 13.2, 14.7, 15.3, 17.4, 17.5, 18.1, 20.3, 20.8, 21.2, 21.8, 22.5, 24.6, 25.4, 26.3, 27.2, 29.7, 30.2, 32.1, and 33.0+/−0.4°2theta.
f. In certain embodiments S-BK-5-MAPB Pattern 1B is characterized by any one of embodiments a-e wherein the XRPD pattern includes a peak at 8.7+/−0.4° 2theta.
g. In certain embodiments S-BK-5-MAPB Pattern 1B is characterized by any one of embodiments a-f wherein the XRPD pattern includes a peak at 8.9+/−0.4°2theta.
h. In certain embodiments S-BK-5-MAPB Pattern 1B is characterized by any one of embodiments a-g wherein the XRPD pattern includes a peak at 17.4+/−0.4° 2theta.
i. In certain embodiments S-BK-5-MAPB Pattern 1B is characterized by any one of embodiments a-h wherein the XRPD pattern includes a peak at 17.5+/−0.4° 2theta.
j. In certain embodiments S-BK-5-MAPB Pattern 1B is characterized by any one of embodiments a-i wherein the XRPD pattern includes a peak at 18.1+/−0.4°2theta.
k. In certain embodiments S-BK-5-MAPB Pattern 1B is characterized by any one of embodiments a-i wherein the XRPD pattern includes a peak at 20.3+/−0.4°2theta.
l. In certain embodiments S-BK-5-MAPB Pattern 1B is characterized by any one of embodiments a-i wherein the XRPD pattern includes a peak at 20.8+/−0.4°2theta.
m. In certain embodiments S-BK-5-MAPB Pattern 1B is characterized by any one of embodiments a-i wherein the XRPD pattern includes a peak at 21.8+/−0.4°2theta.
n. In certain embodiments S-BK-5-MAPB Pattern 1B is characterized by any one of embodiments a-i wherein the XRPD pattern includes a peak at 25.4+/−0.4°2theta.
o. In certain embodiments S-BK-5-MAPB Pattern 1B is characterized by any one of embodiments a-i wherein the XRPD pattern includes a peak at 26.3+/−0.4°2theta.
p. In certain embodiments S-BK-5-MAPB Pattern 1B is characterized by any one of embodiments a-o wherein the peaks are within +/−0.3°2theta.
q. In certain embodiments S-BK-5-MAPB Pattern 1B is characterized by any one of embodiments a-o wherein the peaks are within +/−0.2°2theta.
r. In certain embodiments S-BK-5-MAPB Pattern 1B is characterized by any one of embodiments a-q wherein at least three of the recited peaks have a relative peak intensity of at least 10%.
s. In certain embodiments S-BK-5-MAPB Pattern 1B is characterized by any one of embodiments a-q wherein at least three of the recited peaks have a relative peak intensity of at least 20%.
t. In certain embodiments S-BK-5-MAPB Pattern 1B is characterized by any one of embodiments a-q wherein at least four of the recited peaks have a relative peak intensity of at least 10%.
u. In certain embodiments S-BK-5-MAPB Pattern 1B is characterized by any one of embodiments a-q wherein at least four of the recited peaks have a relative peak intensity of at least 20%.
v. In certain embodiments S-BK-5-MAPB Pattern 1B is characterized by any one of embodiments a-q wherein at least five of the recited peaks have a relative peak intensity of at least 10%.
w. In certain embodiments S-BK-5-MAPB Pattern 1B is characterized by any one of embodiments a-q wherein at least six of the recited peaks have a relative peak intensity of at least 20%.

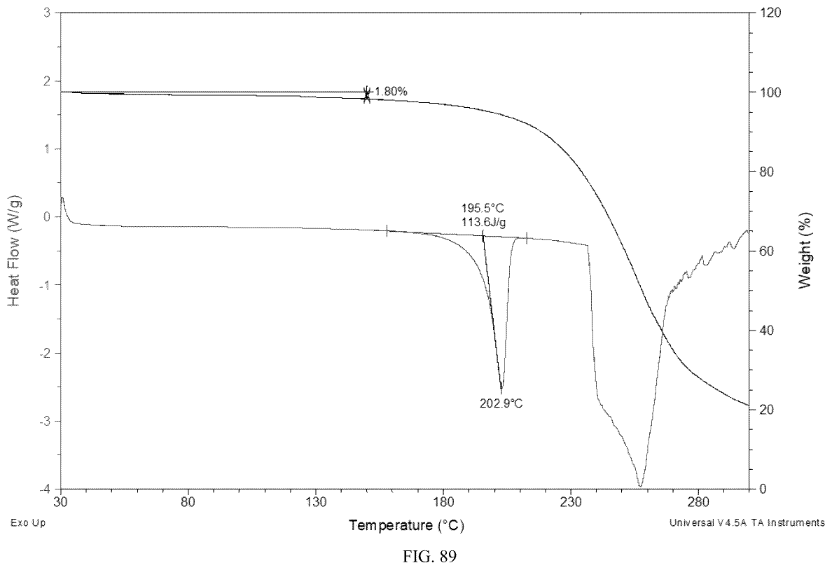

In certain embodiments S-BK-5-MAPB Pattern 1B is characterized by the XRPD diffractogram in FIG. 63 and/or the DSC graph shown in FIG. 89.

H₂SO₄ Salt

In certain aspects the invention provides S-BK-5-MAPB as H₂SO₄ salt. In certain embodiments the S-BK-5-MAPB salt is a stable morphic form denoted Pattern 3A.
a. In certain embodiments S-BK-5-MAPB Pattern 3A is characterized by an XRPD pattern with three or more peaks selected from 11.9, 13.6, 17.3, 17.7, 19.8, 21.4, 21.7, 22.6, 22.8, 24.1, 24.4, 25.9, 26.9, 27.1, 28.9, and 31.3+/−0.4°2theta.
b. In certain embodiments S-BK-5-MAPB Pattern 3A is characterized by an XRPD pattern with four or more peaks selected from 11.9, 13.6, 17.3, 17.7, 19.8, 21.4, 21.7, 22.6, 22.8, 24.1, 24.4, 25.9, 26.9, 27.1, 28.9, and 31.3+/−0.4°2theta.
c. In certain embodiments S-BK-5-MAPB Pattern 3A is characterized by an XRPD pattern with five or more peaks selected from 11.9, 13.6, 17.3, 17.7, 19.8, 21.4, 21.7, 22.6, 22.8, 24.1, 24.4, 25.9, 26.9, 27.1, 28.9, and 31.3+/−0.4°2theta.
d. In certain embodiments S-BK-5-MAPB Pattern 3A is characterized by an XRPD pattern with six or more peaks selected from 11.9, 13.6, 17.3, 17.7, 19.8, 21.4, 21.7, 22.6, 22.8, 24.1, 24.4, 25.9, 26.9, 27.1, 28.9, and 31.3+/−0.4°2theta.
e. In certain embodiments S-BK-5-MAPB Pattern 3A is characterized by an XRPD pattern with seven or more peaks selected from 11.9, 13.6, 17.3, 17.7, 19.8, 21.4, 21.7, 22.6, 22.8, 24.1, 24.4, 25.9, 26.9, 27.1, 28.9, and 31.3+/−0.4° 2theta.
f. In certain embodiments S-BK-5-MAPB Pattern 3A is characterized by any one of embodiments a-e wherein the XRPD pattern includes a peak at 11.9+/−0.4° 2theta.
g. In certain embodiments S-BK-5-MAPB Pattern 3A is characterized by any one of embodiments a-f wherein the XRPD pattern includes a peak at 13.6+/−0.4° 2theta.
h. In certain embodiments S-BK-5-MAPB Pattern 3A is characterized by any one of embodiments a-g wherein the XRPD pattern includes a peak at 17.7+/−0.4° 2theta.
i. In certain embodiments S-BK-5-MAPB Pattern 3A is characterized by any one of embodiments a-h wherein the XRPD pattern includes a peak at 19.8+/−0.4° 2theta.
j. In certain embodiments S-BK-5-MAPB Pattern 3A is characterized by any one of embodiments a-i wherein the XRPD pattern includes a peak at 21.4+/−0.4°2theta.
k. In certain embodiments S-BK-5-MAPB Pattern 3A is characterized by any one of embodiments a-i wherein the XRPD pattern includes a peak at 22.6+/−0.4°2theta.
l. In certain embodiments S-BK-5-MAPB Pattern 3A is characterized by any one of embodiments a-i wherein the XRPD pattern includes a peak at 24.1+/−0.4°2theta.
m. In certain embodiments S-BK-5-MAPB Pattern 3A is characterized by any one of embodiments a-i wherein the XRPD pattern includes a peak at 24.4+/−0.4°2theta.
n. In certain embodiments S-BK-5-MAPB Pattern 3A is characterized by any one of embodiments a-i wherein the XRPD pattern includes a peak at 27.1+/−0.4°2theta.
o. In certain embodiments S-BK-5-MAPB Pattern 3A is characterized by any one of embodiments a-i wherein the XRPD pattern includes a peak at 28.9+/−0.4°2theta.
p. In certain embodiments S-BK-5-MAPB Pattern 3A is characterized by any one of embodiments a-o wherein the peaks are within +/−0.3°2theta.
q. In certain embodiments S-BK-5-MAPB Pattern 3A is characterized by any one of embodiments a-o wherein the peaks are within +/−0.2°2theta.
r. In certain embodiments S-BK-5-MAPB Pattern 3A is characterized by any one of embodiments a-q wherein at least three of the recited peaks have a relative peak intensity of at least 10%.
s. In certain embodiments S-BK-5-MAPB Pattern 3A is characterized by any one of embodiments a-q wherein at least three of the recited peaks have a relative peak intensity of at
t. In certain embodiments S-BK-5-MAPB Pattern 3A is characterized by any one of embodiments a-q wherein at least four of the recited peaks have a relative peak intensity of at least 10%.
u. In certain embodiments S-BK-5-MAPB Pattern 3A is characterized by any one of embodiments a-q wherein at least four of the recited peaks have a relative peak intensity of at least 20%.
v. In certain embodiments S-BK-5-MAPB Pattern 3A is characterized by any one of embodiments a-q wherein at least five of the recited peaks have a relative peak intensity of at least 10%.
w. In certain embodiments S-BK-5-MAPB Pattern 3A is characterized by any one of embodiments a-q wherein at least six of the recited peaks have a relative peak intensity of at least 20%.

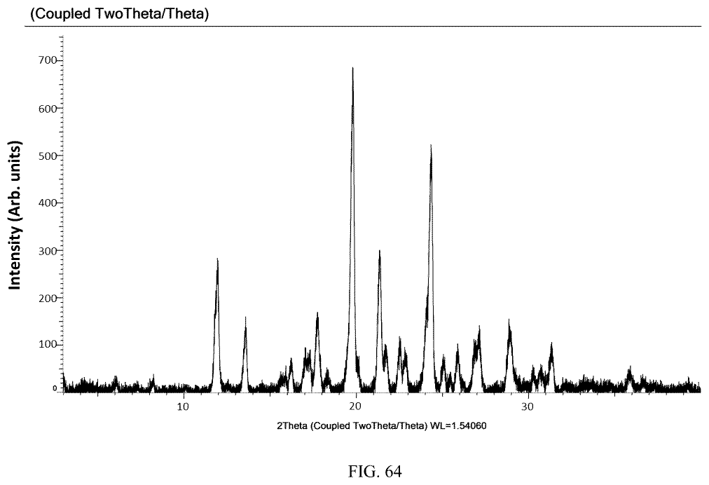

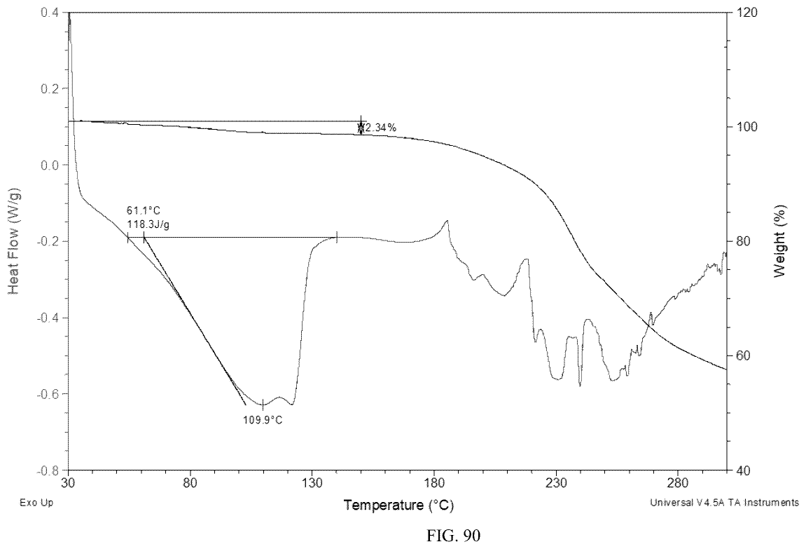

In certain embodiments S-BK-5-MAPB Pattern 3A is characterized by the XRPD diffractogram in FIG. 64 and/or the DSC graph shown in FIG. 90.

### Maleic Salt

In certain aspects the invention provides S-BK-5-MAPB as maleic salt. In certain embodiments the S-BK-5-MAPB salt is a stable morphic form denoted Pattern 10A.
a. In certain embodiments S-BK-5-MAPB Pattern 10A is characterized by an XRPD pattern with three or more peaks selected from 10.0, 12.9, 17.3, 19.1, 19.9, 21.1, 23.4, 23.9, 25.9, 27.4, 28.8, 31.6, 33.1, 35.0, and 38.2+/−0.4°2theta.
b. In certain embodiments S-BK-5-MAPB Pattern 10A is characterized by an XRPD pattern with four or more peaks selected from 10.0, 12.9, 17.3, 19.1, 19.9, 21.1, 23.4, 23.9, 25.9, 27.4, 28.8, 31.6, 33.1, 35.0, and 38.2+/−0.4°2theta.
c. In certain embodiments S-BK-5-MAPB Pattern 10A is characterized by an XRPD pattern with five or more peaks selected from 10.0, 12.9, 17.3, 19.1, 19.9, 21.1, 23.4, 23.9, 25.9, 27.4, 28.8, 31.6, 33.1, 35.0, and 38.2+/−0.4° 2theta.
d. In certain embodiments S-BK-5-MAPB Pattern 10A is characterized by an XRPD pattern with six or more peaks selected from 10.0, 12.9, 17.3, 19.1, 19.9, 21.1, 23.4, 23.9, 25.9, 27.4, 28.8, 31.6, 33.1, 35.0, and 38.2+/−0.4°2theta.
e. In certain embodiments S-BK-5-MAPB Pattern 10A is characterized by an XRPD pattern with seven or more peaks selected from 10.0, 12.9, 17.3, 19.1, 19.9, 21.1, 23.4, 23.9, 25.9, 27.4, 28.8, 31.6, 33.1, 35.0, and 38.2+/−0.4° 2theta.
f. In certain embodiments S-BK-5-MAPB Pattern 10A is characterized by any one of embodiments a-e wherein the XRPD pattern includes a peak at 10.0+/−0.4° 2theta.
g. In certain embodiments S-BK-5-MAPB Pattern 10A is characterized by any one of embodiments a-f wherein the XRPD pattern includes a peak at 12.9+/−0.4°2theta.
h. In certain embodiments S-BK-5-MAPB Pattern 10A is characterized by any one of embodiments a-g wherein the XRPD pattern includes a peak at 17.3+/−0.4°2theta.
i. In certain embodiments S-BK-5-MAPB Pattern 10A is characterized by any one of embodiments a-h wherein the XRPD pattern includes a peak at 19.1+/−0.4°2theta.
j. In certain embodiments S-BK-5-MAPB Pattern 10A is characterized by any one of embodiments a-i wherein the XRPD pattern includes a peak at 19.9+/−0.4°2theta.
k. In certain embodiments S-BK-5-MAPB Pattern 10A is characterized by any one of embodiments a-i wherein the XRPD pattern includes a peak at 23.4+/−0.4°2theta.
l. In certain embodiments S-BK-5-MAPB Pattern 10A is characterized by any one of embodiments a-i wherein the XRPD pattern includes a peak at 23.9+/−0.4°2theta.
m. In certain embodiments S-BK-5-MAPB Pattern 10A is characterized by any one of embodiments a-i wherein the XRPD pattern includes a peak at 27.4+/−0.4°2theta.
n. In certain embodiments S-BK-5-MAPB Pattern 10A is characterized by any one of embodiments a-i wherein the XRPD pattern includes a peak at 27.4+/−0.4°2theta.
o. In certain embodiments S-BK-5-MAPB Pattern 10A is characterized by any one of embodiments a-i wherein the XRPD pattern includes a peak at 28.8+/−0.4°2theta.
p. In certain embodiments S-BK-5-MAPB Pattern 10A is characterized by any one of embodiments a-o wherein the peaks are within +/−0.3°2theta.
q. In certain embodiments S-BK-5-MAPB Pattern 10A is characterized by any one of embodiments a-o wherein the peaks are within +/−0.2° 2theta.
r. In certain embodiments S-BK-5-MAPB Pattern 10A is characterized by any one of embodiments a-q wherein at least three of the recited peaks have a relative peak intensity of at least 10%.
s. In certain embodiments S-BK-5-MAPB Pattern 10A is characterized by any one of embodiments a-q wherein at least three of the recited peaks have a relative peak intensity of at
t. In certain embodiments S-BK-5-MAPB Pattern 10A is characterized by any one of embodiments a-q wherein at least four of the recited peaks have a relative peak intensity of at least 10%.
u. In certain embodiments S-BK-5-MAPB Pattern 10A is characterized by any one of embodiments a-q wherein at least four of the recited peaks have a relative peak intensity of at least 20%.
v. In certain embodiments S-BK-5-MAPB Pattern 10A is characterized by any one of embodiments a-q wherein at least five of the recited peaks have a relative peak intensity of at least 10%.
w. In certain embodiments S-BK-5-MAPB Pattern 10A is characterized by any one of embodiments a-q wherein at least six of the recited peaks have a relative peak intensity of at least 20%.

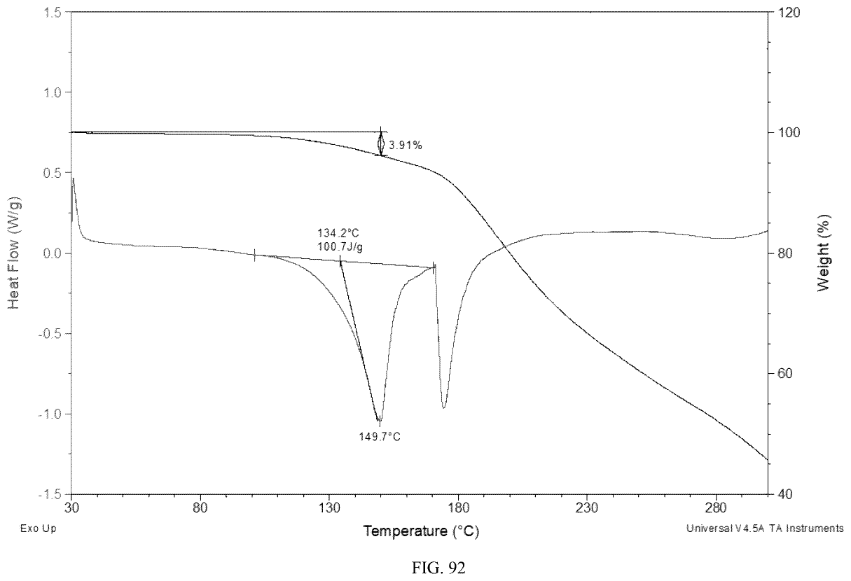

In certain embodiments S-BK-5-MAPB Pattern 10A is characterized by the XRPD diffractogram in FIG. 66 and/or the DSC graph shown in FIG. 92.

### Malic Salt

In certain aspects the invention provides S-BK-5-MAPB as malic salt. In certain embodiments the S-BK-5-MAPB salt is a stable morphic form denoted Pattern 11A.
a. In certain embodiments S-BK-5-MAPB Pattern 11A is characterized by an XRPD pattern with three or more peaks selected from 6.3, 12.5, 13.5, 16.4, 17.5, 17.8, 18.0, 18.9, 20.4, 214, 21.7, 23.6, 24.7, 25.3, 25.6, 26.7, 27.0, 27.4, 28.0, 28.2, and 32.9+/−0.4°2theta.
b. In certain embodiments S-BK-5-MAPB Pattern 11A is characterized by an XRPD pattern with four or more peaks selected from 6.3, 12.5, 13.5, 16.4, 17.5, 17.8, 18.0, 18.9, 20.4, 214, 21.7, 23.6, 24.7, 25.3, 25.6, 26.7, 27.0, 27.4, 28.0, 28.2, and 32.9+/−0.4°2theta.
c. In certain embodiments S-BK-5-MAPB Pattern 11A is characterized by an XRPD pattern with five or more peaks selected from 6.3, 12.5, 13.5, 16.4, 17.5, 17.8, 18.0, 18.9, 20.4, 214, 21.7, 23.6, 24.7, 25.3, 25.6, 26.7, 27.0, 27.4, 28.0, 28.2, and 32.9+/−0.4°2theta.
d. In certain embodiments S-BK-5-MAPB Pattern 11A is characterized by an XRPD pattern with six or more peaks selected from 6.3, 12.5, 13.5, 16.4, 17.5, 17.8, 18.0, 18.9, 20.4, 214, 21.7, 23.6, 24.7, 25.3, 25.6, 26.7, 27.0, 27.4, 28.0, 28.2, and 32.9+/−0.4°2theta.
e. In certain embodiments S-BK-5-MAPB Pattern 11A is characterized by an XRPD pattern with seven or more peaks selected from 6.3, 12.5, 13.5, 16.4, 17.5, 17.8, 18.0, 18.9, 20.4, 214, 21.7, 23.6, 24.7, 25.3, 25.6, 26.7, 27.0, 27.4, 28.0, 28.2, and 32.9+/−0.4° 2theta.
f. In certain embodiments S-BK-5-MAPB Pattern 11A is characterized by any one of embodiments a-e wherein the XRPD pattern includes a peak at 12.5+/−0.4° 2theta.
g. In certain embodiments S-BK-5-MAPB Pattern 11A is characterized by any one of embodiments a-f wherein the XRPD pattern includes a peak at 16.4+/−0.4°2theta.
h. In certain embodiments S-BK-5-MAPB Pattern 11A is characterized by any one of embodiments a-g wherein the XRPD pattern includes a peak at 17.5+/−0.4°2theta.
i. In certain embodiments S-BK-5-MAPB Pattern 11A is characterized by any one of embodiments a-h wherein the XRPD pattern includes a peak at 18.0+/−0.4°2theta.
j. In certain embodiments S-BK-5-MAPB Pattern 11A is characterized by any one of embodiments a-i wherein the XRPD pattern includes a peak at 18.9+/−0.4°2theta.
k. In certain embodiments S-BK-5-MAPB Pattern 11A is characterized by any one of embodiments a-i wherein the XRPD pattern includes a peak at 20.4+/−0.4°2theta.
l. In certain embodiments S-BK-5-MAPB Pattern 11A is characterized by any one of embodiments a-i wherein the XRPD pattern includes a peak at 21.4+/−0.4°2theta.
m. In certain embodiments S-BK-5-MAPB Pattern 11A is characterized by any one of embodiments a-i wherein the XRPD pattern includes a peak at 23.6+/−0.4°2theta.
n. In certain embodiments S-BK-5-MAPB Pattern 11A is characterized by any one of embodiments a-i wherein the XRPD pattern includes a peak at 24.7+/−0.4°2theta.
o. In certain embodiments S-BK-5-MAPB Pattern 11A is characterized by any one of embodiments a-i wherein the XRPD pattern includes a peak at 27.4+/−0.4°2theta.
p. In certain embodiments S-BK-5-MAPB Pattern 11A is characterized by any one of embodiments a-o wherein the peaks are within +/−0.3°2theta.
q. In certain embodiments S-BK-5-MAPB Pattern 11A is characterized by any one of embodiments a-o wherein the peaks are within +/−0.2°2theta.
r. In certain embodiments S-BK-5-MAPB Pattern 11A is characterized by any one of embodiments a-q wherein at least three of the recited peaks have a relative peak intensity of at least 10%.
s. In certain embodiments S-BK-5-MAPB Pattern 11A is characterized by any one of embodiments a-q wherein at least three of the recited peaks have a relative peak intensity of at least 20%.
t. In certain embodiments S-BK-5-MAPB Pattern 11A is characterized by any one of embodiments a-q wherein at least four of the recited peaks have a relative peak intensity of at least 10%.
u. In certain embodiments S-BK-5-MAPB Pattern 11A is characterized by any one of embodiments a-q wherein at least four of the recited peaks have a relative peak intensity of at least 20%.
v. In certain embodiments S-BK-5-MAPB Pattern 11A is characterized by any one of embodiments a-q wherein at least five of the recited peaks have a relative peak intensity of at least 10%.
w. In certain embodiments S-BK-5-MAPB Pattern 11A is characterized by any one of embodiments a-q wherein at least six of the recited peaks have a relative peak intensity of at least 20%.

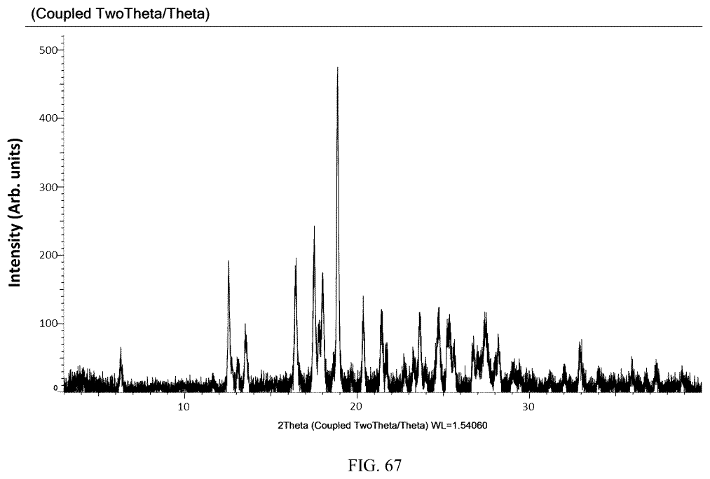

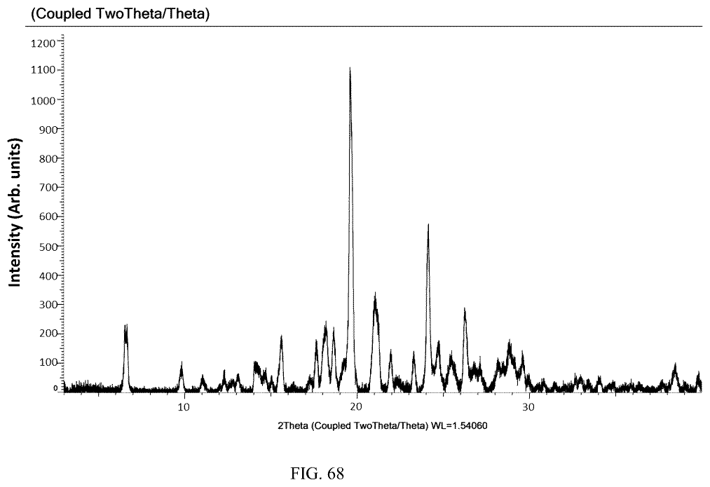

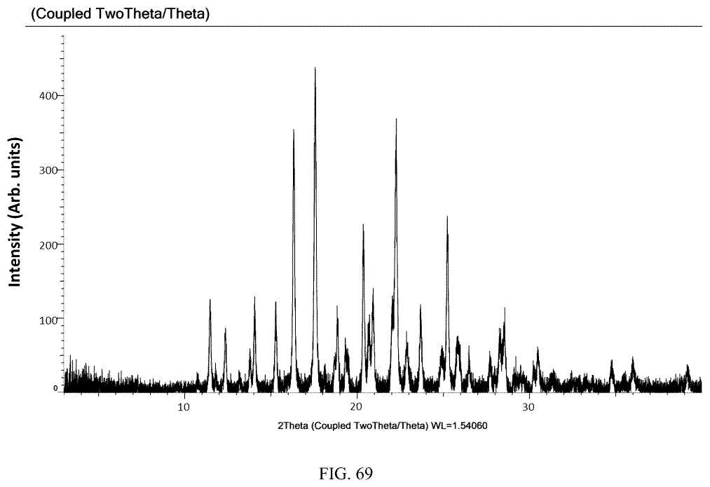

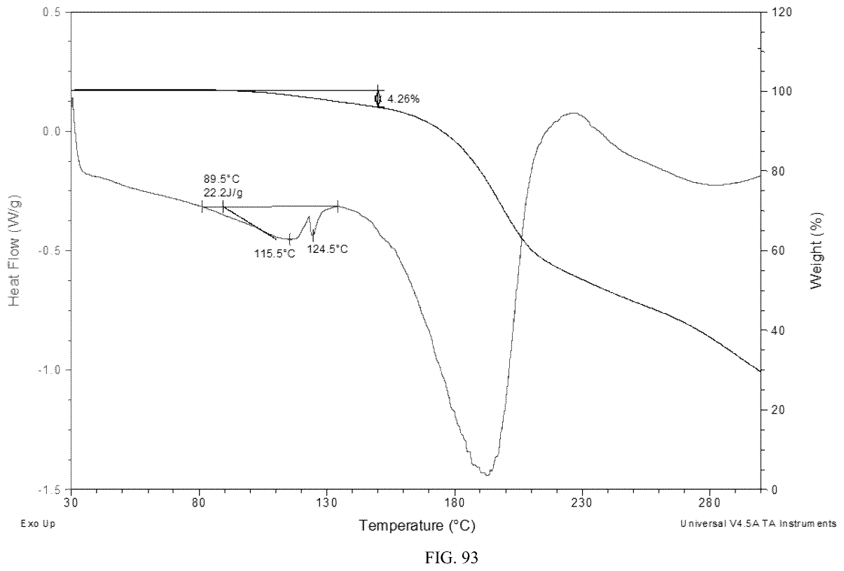

In certain embodiments S-BK-5-MAPB Pattern 11A is characterized by the XRPD diffractogram in FIG. 67 and/or the DSC graph shown in FIG. 93.

### Fumaric Salt

In certain aspects the invention provides S-BK-5-MAPB as fumaric salt. In certain embodiments the S-BK-5-MAPB salt is a stable morphic form denoted Pattern 13A.
a. In certain embodiments S-BK-5-MAPB Pattern 13A is characterized by an XRPD pattern with three or more peaks selected from 6.5, 6.6, 15.6, 17.6, 18.2, 18.6, 19.6, 21.0, 22.0, 23.3, 24.1, 24.7, 26.2, 28.8, and 29.6+/−0.4°2theta.
b. In certain embodiments S-BK-5-MAPB Pattern 13A is characterized by an XRPD pattern with four or more peaks selected from 66.5, 6.6, 15.6, 17.6, 18.2, 18.6, 19.6, 21.0, 22.0, 23.3, 24.1, 24.7, 26.2, 28.8, and 29.6+/−0.4° 2theta.
c. In certain embodiments S-BK-5-MAPB Pattern 13A is characterized by an XRPD pattern with five or more peaks selected from 6.5, 6.6, 15.6, 17.6, 18.2, 18.6, 19.6, 21.0, 22.0, 23.3, 24.1, 24.7, 26.2, 28.8, and 29.6+/−0.4°2theta.
d. In certain embodiments S-BK-5-MAPB Pattern 13A is characterized by an XRPD pattern with six or more peaks selected from 6.5, 6.6, 15.6, 17.6, 18.2, 18.6, 19.6, 21.0, 22.0, 23.3, 24.1, 24.7, 26.2, 28.8, and 29.6+/−0.4° 2theta.
e. In certain embodiments S-BK-5-MAPB Pattern 13A is characterized by an XRPD pattern with seven or more peaks selected from 6.5, 6.6, 15.6, 17.6, 18.2, 18.6, 19.6, 21.0, 22.0, 23.3, 24.1, 24.7, 26.2, 28.8, and 29.6+/−0.4° 2theta.
f. In certain embodiments S-BK-5-MAPB Pattern 13A is characterized by any one of embodiments a-e wherein the XRPD pattern includes a peak at 6.5+/−0.4° 2theta.
g. In certain embodiments S-BK-5-MAPB Pattern 13A is characterized by any one of embodiments a-f wherein the XRPD pattern includes a peak at 6.6+/−0.4°2theta.
h. In certain embodiments S-BK-5-MAPB Pattern 13A is characterized by any one of embodiments a-g wherein the XRPD pattern includes a peak at 18.2+/−0.4°2theta.
i. In certain embodiments S-BK-5-MAPB Pattern 13A is characterized by any one of embodiments a-h wherein the XRPD pattern includes a peak at 18.6+/−0.4°2theta.
j. In certain embodiments S-BK-5-MAPB Pattern 13A is characterized by any one of embodiments a-i wherein the XRPD pattern includes a peak at 19.6+/−0.4° 2theta.
k. In certain embodiments S-BK-5-MAPB Pattern 13A is characterized by any one of embodiments a-i wherein the XRPD pattern includes a peak at 21.0+/−0.4°2theta.
l. In certain embodiments S-BK-5-MAPB Pattern 13A is characterized by any one of embodiments a-i wherein the XRPD pattern includes a peak at 24.1+/−0.4° 2theta.
m. In certain embodiments S-BK-5-MAPB Pattern 13A is characterized by any one of embodiments a-i wherein the XRPD pattern includes a peak at 24.7+/−0.4°2theta.
n. In certain embodiments S-BK-5-MAPB Pattern 13A is characterized by any one of embodiments a-i wherein the XRPD pattern includes a peak at 26.2+/−0.4° 2theta.
o. In certain embodiments S-BK-5-MAPB Pattern 13A is characterized by any one of embodiments a-i wherein the XRPD pattern includes a peak at 28.8+/−0.4°2theta.
p. In certain embodiments S-BK-5-MAPB Pattern 13A is characterized by any one of embodiments a-o wherein the peaks are within +/−0.3°2theta.
q. In certain embodiments S-BK-5-MAPB Pattern 13A is characterized by any one of embodiments a-o wherein the peaks are within +/−0.2°2theta.
r. In certain embodiments S-BK-5-MAPB Pattern 13A is characterized by any one of embodiments a-q wherein at least three of the recited peaks have a relative peak intensity of at least 10%.
s. In certain embodiments S-BK-5-MAPB Pattern 13A is characterized by any one of embodiments a-q wherein at least three of the recited peaks have a relative peak intensity of at least 20%.
t. In certain embodiments S-BK-5-MAPB Pattern 13A is characterized by any one of embodiments a-q wherein at least four of the recited peaks have a relative peak intensity of at least 10%.
u. In certain embodiments S-BK-5-MAPB Pattern 13A is characterized by any one of embodiments a-q wherein at least four of the recited peaks have a relative peak intensity of at least 20%.
v. In certain embodiments S-BK-5-MAPB Pattern 13A is characterized by any one of embodiments a-q wherein at least five of the recited peaks have a relative peak intensity of at least 10%.
w. In certain embodiments S-BK-5-MAPB Pattern 13A is characterized by any one of embodiments a-q wherein at least six of the recited peaks have a relative peak intensity of at least 20%.

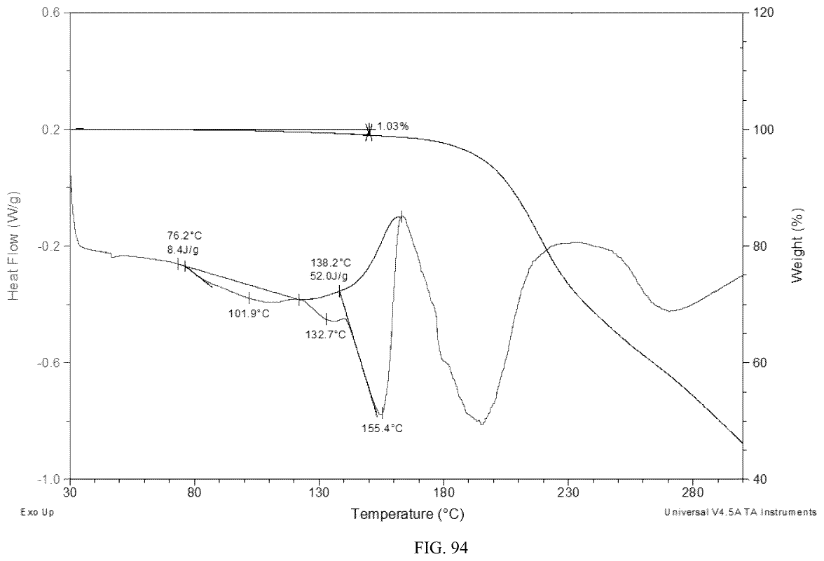

In certain embodiments S-BK-5-MAPB Pattern 13A is characterized by the XRPD diffractogram in FIG. 68 and/or the DSC graph shown in FIG. 94.

### Benzoic Salt

In certain aspects the invention provides S-BK-5-MAPB as benzoic salt. In certain embodiments the S-BK-5-MAPB salt is a stable morphic form denoted Pattern 14A.
a. In certain embodiments S-BK-5-MAPB Pattern 14A is characterized by an XRPD pattern with three or more peaks selected from 11.5, 12.4, 13.8, 14.1, 15.3, 16.3, 17.6, 18.7, 18.9, 19.3, 20.4, 20.7, 20.9, 22.1, 22.3, 22.9, 23.7, 25.2, 25.9, 26.5, 27.7, 28.3, 28.5, 30.5, and 36.0+/−0.4° 2theta.
b. In certain embodiments S-BK-5-MAPB Pattern 14A is characterized by an XRPD pattern with four or more peaks selected from 11.5, 12.4, 13.8, 14.1, 15.3, 16.3, 17.6, 18.7, 18.9, 19.3, 20.4, 20.7, 20.9, 22.1, 22.3, 22.9, 23.7, 25.2, 25.9, 26.5, 27.7, 28.3, 28.5, 30.5, and 36.0+/−0.4° 2theta.
c. In certain embodiments S-BK-5-MAPB Pattern 14A is characterized by an XRPD pattern with five or more peaks selected from 11.5, 12.4, 13.8, 14.1, 15.3, 16.3, 17.6, 18.7, 18.9, 19.3, 20.4, 20.7, 20.9, 22.1, 22.3, 22.9, 23.7, 25.2, 25.9, 26.5, 27.7, 28.3, 28.5, 30.5, and 36.0+/−0.4° 2theta.
d. In certain embodiments S-BK-5-MAPB Pattern 14A is characterized by an XRPD pattern with six or more peaks selected from 11.5, 12.4, 13.8, 14.1, 15.3, 16.3, 17.6, 18.7, 18.9, 19.3, 20.4, 20.7, 20.9, 22.1, 22.3, 22.9, 23.7, 25.2, 25.9, 26.5, 27.7, 28.3, 28.5, 30.5, and 36.0+/−0.4° 2theta.
e. In certain embodiments S-BK-5-MAPB Pattern 14A is characterized by an XRPD pattern with seven or more peaks selected from 11.5, 12.4, 13.8, 14.1, 15.3, 16.3, 17.6, 18.7, 18.9, 19.3, 20.4, 20.7, 20.9, 22.1, 22.3, 22.9, 23.7, 25.2, 25.9, 26.5, 27.7, 28.3, 28.5, 30.5, and 36.0+/−0.4° 2theta.
f. In certain embodiments S-BK-5-MAPB Pattern 14A is characterized by any one of embodiments a-e wherein the XRPD pattern includes a peak at 11.5+/−0.4° 2theta.
g. In certain embodiments S-BK-5-MAPB Pattern 14A is characterized by any one of embodiments a-f wherein the XRPD pattern includes a peak at 12.4+/−0.4° 2theta.
h. In certain embodiments S-BK-5-MAPB Pattern 14A is characterized by any one of embodiments a-g wherein the XRPD pattern includes a peak at 14.1+/−0.4°2theta.
i. In certain embodiments S-BK-5-MAPB Pattern 14A is characterized by any one of embodiments a-h wherein the XRPD pattern includes a peak at 15.3+/−0.4°2theta.
j. In certain embodiments S-BK-5-MAPB Pattern 14A is characterized by any one of embodiments a-i wherein the XRPD pattern includes a peak at 16.3+/−0.4°2theta.
k. In certain embodiments S-BK-5-MAPB Pattern 14A is characterized by any one of embodiments a-i wherein the XRPD pattern includes a peak at 17.6+/−0.4° 2theta.
l. In certain embodiments S-BK-5-MAPB Pattern 14A is characterized by any one of embodiments a-i wherein the XRPD pattern includes a peak at 18.9+/−0.4°2theta.
m. In certain embodiments S-BK-5-MAPB Pattern 14A is characterized by any one of embodiments a-i wherein the XRPD pattern includes a peak at 20.4+/−0.4°2theta.
n. In certain embodiments S-BK-5-MAPB Pattern 14A is characterized by any one of embodiments a-i wherein the XRPD pattern includes a peak at 22.3+/−0.4°2theta.
o. In certain embodiments S-BK-5-MAPB Pattern 14A is characterized by any one of embodiments a-i wherein the XRPD pattern includes a peak at 25.2+/−0.4° 2theta.
p. In certain embodiments S-BK-5-MAPB Pattern 14A is characterized by any one of embodiments a-o wherein the peaks are within +/−0.3°2theta.
q. In certain embodiments S-BK-5-MAPB Pattern 14A is characterized by any one of embodiments a-o wherein the peaks are within +/−0.2°2theta.
r. In certain embodiments S-BK-5-MAPB Pattern 14A is characterized by any one of embodiments a-q wherein at least three of the recited peaks have a relative peak intensity of at least 10%.
s. In certain embodiments S-BK-5-MAPB Pattern 14A is characterized by any one of embodiments a-q wherein at least three of the recited peaks have a relative peak intensity of at least 20%.
t. In certain embodiments S-BK-5-MAPB Pattern 14A is characterized by any one of embodiments a-q wherein at least four of the recited peaks have a relative peak intensity of at least 10%.
u. In certain embodiments S-BK-5-MAPB Pattern 14A is characterized by any one of embodiments a-q wherein at least four of the recited peaks have a relative peak intensity of at least 20%.
v. In certain embodiments S-BK-5-MAPB Pattern 14A is characterized by any one of embodiments a-q wherein at least five of the recited peaks have a relative peak intensity of at least 10%.
w. In certain embodiments S-BK-5-MAPB Pattern 14A is characterized by any one of embodiments a-q wherein at least six of the recited peaks have a relative peak intensity of at least 20%.

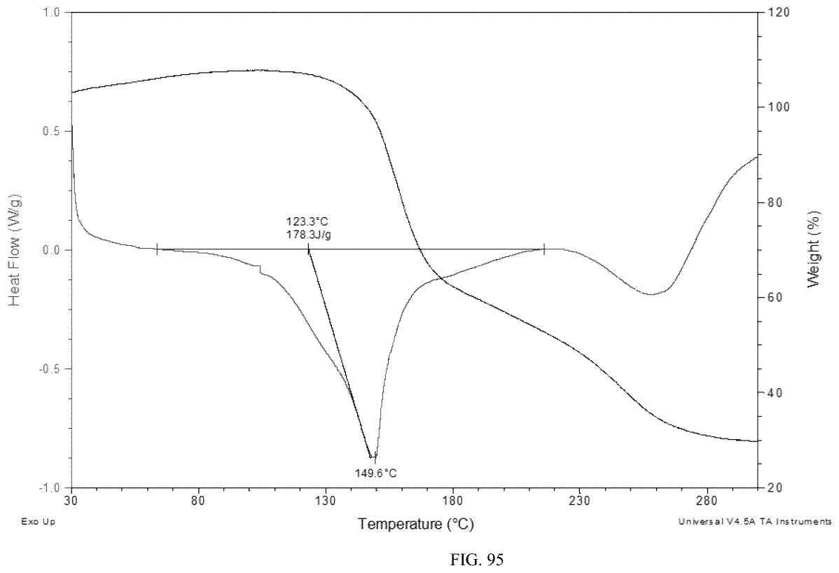

In certain embodiments S-BK-5-MAPB Pattern 14A is characterized by the XRPD diffractogram in FIG. 69 and/or the DSC graph shown in FIG. 95.

### Salicylic Salt

### Pattern 15A

In certain aspects the invention provides S-BK-5-MAPB as salicylic salt. In certain embodiments the S-BK-5-MAPB salt is a stable morphic form denoted Pattern 15A.
a. In certain embodiments S-BK-5-MAPB Pattern 15A is characterized by an XRPD pattern with three or more peaks selected from 8.3, 10.9, 16.6, 17.1, 18.1, 18.3, 21.8, 24.6, 25.0, and 33.5+/−0.4° 2theta.
b. In certain embodiments S-BK-5-MAPB Pattern 15A is characterized by an XRPD pattern with four or more peaks selected from 8.3, 10.9, 16.6, 17.1, 18.1, 18.3, 21.8, 24.6, 25.0, and 33.5+/−0.4° 2theta.
c. In certain embodiments S-BK-5-MAPB Pattern 15A is characterized by an XRPD pattern with five or more peaks selected from 8.3, 10.9, 16.6, 17.1, 18.1, 18.3, 21.8, 24.6, 25.0, and 33.5+/−0.4° 2theta.
d. In certain embodiments S-BK-5-MAPB Pattern 15A is characterized by an XRPD pattern with six or more peaks selected from 8.3, 10.9, 16.6, 17.1, 18.1, 18.3, 21.8, 24.6, 25.0, and 33.5+/−0.4° 2theta.
e. In certain embodiments S-BK-5-MAPB Pattern 15A is characterized by an XRPD pattern with seven or more peaks selected from 8.3, 10.9, 16.6, 17.1, 18.1, 18.3, 21.8, 24.6, 25.0, and 33.5+/−0.4° 2theta.
f. In certain embodiments S-BK-5-MAPB Pattern 15A is characterized by any one of embodiments a-e wherein the XRPD pattern includes a peak at 8.3+/−0.4° 2theta.
g. In certain embodiments S-BK-5-MAPB Pattern 15A is characterized by any one of embodiments a-f wherein the XRPD pattern includes a peak at 10.9+/−0.4°2theta.
h. In certain embodiments S-BK-5-MAPB Pattern 15A is characterized by any one of embodiments a-g wherein the XRPD pattern includes a peak at 16.6+/−0.4°2theta.
i. In certain embodiments S-BK-5-MAPB Pattern 15A is characterized by any one of embodiments a-h wherein the XRPD pattern includes a peak at 16.8+/−0.4°2theta.
j. In certain embodiments S-BK-5-MAPB Pattern 15A is characterized by any one of embodiments a-i wherein the XRPD pattern includes a peak at 17.1+/−0.4°2theta.
k. In certain embodiments S-BK-5-MAPB Pattern 15A is characterized by any one of embodiments a-i wherein the XRPD pattern includes a peak at 17.5+/−0.4°2theta.
l. In certain embodiments S-BK-5-MAPB Pattern 15A is characterized by any one of embodiments a-i wherein the XRPD pattern includes a peak at 18.1+/−0.4°2theta.
m. In certain embodiments S-BK-5-MAPB Pattern 15A is characterized by any one of embodiments a-i wherein the XRPD pattern includes a peak at 21.8+/−0.4°2theta.
n. In certain embodiments S-BK-5-MAPB Pattern 15A is characterized by any one of embodiments a-i wherein the XRPD pattern includes a peak at 24.6+/−0.4°2theta.
o. In certain embodiments S-BK-5-MAPB Pattern 15A is characterized by any one of embodiments a-i wherein the XRPD pattern includes a peak at 25.0+/−0.4°2theta.
p. In certain embodiments S-BK-5-MAPB Pattern 15A is characterized by any one of embodiments a-o wherein the peaks are within +/−0.3°2theta.
q. In certain embodiments S-BK-5-MAPB Pattern 15A is characterized by any one of embodiments a-o wherein the peaks are within +/−0.2° 2theta.
r. In certain embodiments S-BK-5-MAPB Pattern 15A is characterized by any one of embodiments a-q wherein at least three of the recited peaks have a relative peak intensity of at least 10%.
s. In certain embodiments S-BK-5-MAPB Pattern 15A is characterized by any one of embodiments a-q wherein at least three of the recited peaks have a relative peak intensity of at least 20%.
t. In certain embodiments S-BK-5-MAPB Pattern 15A is characterized by any one of embodiments a-q wherein at least four of the recited peaks have a relative peak intensity of at least 10%.
u. In certain embodiments S-BK-5-MAPB Pattern 15A is characterized by any one of embodiments a-q wherein at least four of the recited peaks have a relative peak intensity of at least 20%.
v. In certain embodiments S-BK-5-MAPB Pattern 15A is characterized by any one of embodiments a-q wherein at least five of the recited peaks have a relative peak intensity of at least 10%.
w. In certain embodiments S-BK-5-MAPB Pattern 15A is characterized by any one of embodiments a-q wherein at least six of the recited peaks have a relative peak intensity of at least 20%.

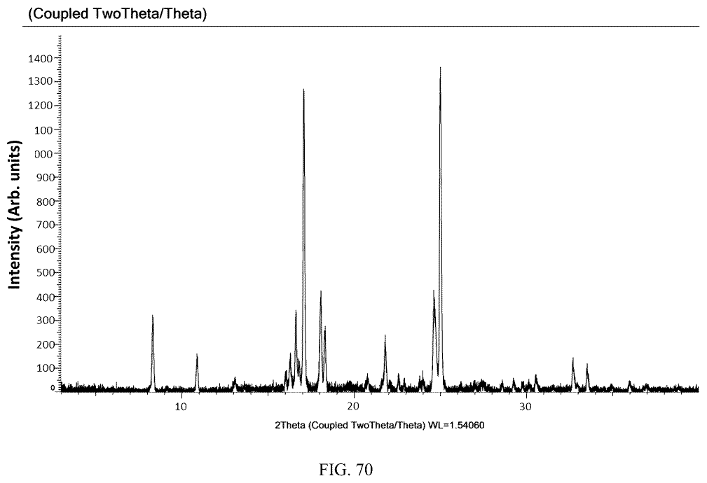

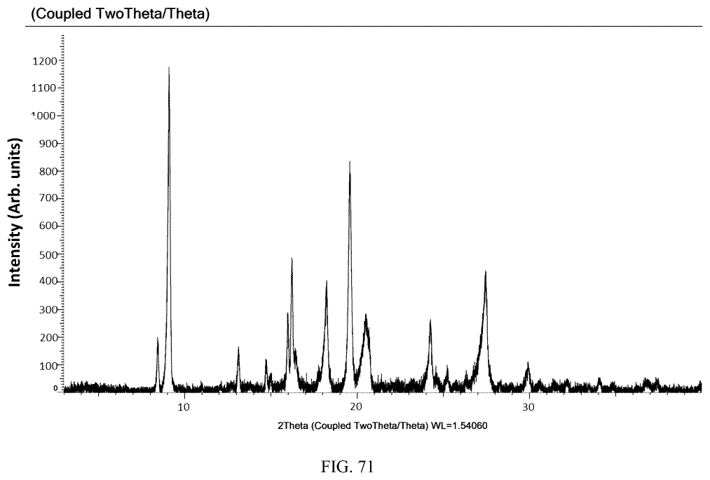

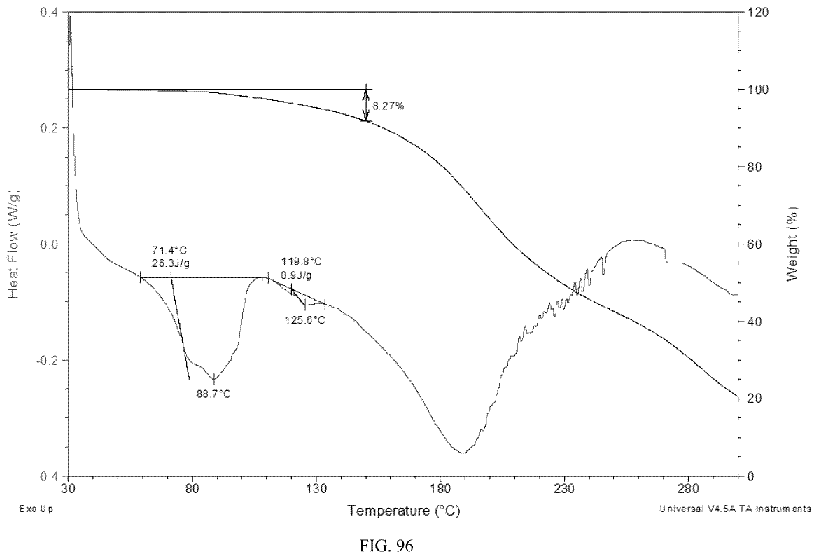

In certain embodiments S-BK-5-MAPB Pattern 15A is characterized by the XRPD diffractogram in FIG. 70 and/or the DSC graph shown in FIG. 96.

### Pattern 15B

In certain aspects the invention provides S-BK-5-MAPB as salicylic salt. In certain embodiments the S-BK-5-MAPB salt is a stable morphic form denoted Pattern 15B.
a. In certain embodiments S-BK-5-MAPB Pattern 15B is characterized by an XRPD pattern with three or more peaks selected from 8.4, 9.1, 13.1, 16.0, 16.2, 16.4, 18.2, 19.6, 20.5, 24.3, and 27.5+/−0.4°2theta.
b. In certain embodiments S-BK-5-MAPB Pattern 15B is characterized by an XRPD pattern with four or more peaks selected from 8.4, 9.1, 13.1, 16.0, 16.2, 16.4, 18.2, 19.6, 20.5, 24.3, and 27.5+/−0.4° 2theta.
c. In certain embodiments S-BK-5-MAPB Pattern 15B is characterized by an XRPD pattern with five or more peaks selected from 8.4, 9.1, 13.1, 16.0, 16.2, 16.4, 18.2, 19.6, 20.5, 24.3, and 27.5+/−0.4°2theta.
d. In certain embodiments S-BK-5-MAPB Pattern 15B is characterized by an XRPD pattern with six or more peaks selected from 8.4, 9.1, 13.1, 16.0, 16.2, 16.4, 18.2, 19.6, 20.5, 24.3, and 27.5+/−0.4° 2theta.
e. In certain embodiments S-BK-5-MAPB Pattern 15B is characterized by an XRPD pattern with seven or more peaks selected from 8.4, 9.1, 13.1, 16.0, 16.2, 16.4, 18.2, 19.6, 20.5, 24.3, and 27.5+/−0.4° 2theta.
f. In certain embodiments S-BK-5-MAPB Pattern 15B is characterized by any one of embodiments a-e wherein the XRPD pattern includes a peak at 8.4+/−0.4° 2theta.
g. In certain embodiments S-BK-5-MAPB Pattern 15B is characterized by any one of embodiments a-f wherein the XRPD pattern includes a peak at 9.1+/−0.4°2theta.
h. In certain embodiments S-BK-5-MAPB Pattern 15B is characterized by any one of embodiments a-g wherein the XRPD pattern includes a peak at 13.1+/−0.4°2theta.
i. In certain embodiments S-BK-5-MAPB Pattern 15B is characterized by any one of embodiments a-h wherein the XRPD pattern includes a peak at 16.0+/−0.4° 2theta.
j. In certain embodiments S-BK-5-MAPB Pattern 15B is characterized by any one of embodiments a-i wherein the XRPD pattern includes a peak at 16.2+/−0.4° 2theta.
k. In certain embodiments S-BK-5-MAPB Pattern 15B is characterized by any one of embodiments a-i wherein the XRPD pattern includes a peak at 18.2+/−0.4°2theta.
l. In certain embodiments S-BK-5-MAPB Pattern 15B is characterized by any one of embodiments a-i wherein the XRPD pattern includes a peak at 19.6+/−0.4°2theta.
m. In certain embodiments S-BK-5-MAPB Pattern 15B is characterized by any one of embodiments a-i wherein the XRPD pattern includes a peak at 20.5+/−0.4°2theta.
n. In certain embodiments S-BK-5-MAPB Pattern 15B is characterized by any one of embodiments a-i wherein the XRPD pattern includes a peak at 24.3+/−0.4°2theta.
o. In certain embodiments S-BK-5-MAPB Pattern 15B is characterized by any one of embodiments a-i wherein the XRPD pattern includes a peak at 27.5+/−0.4°2theta.
p. In certain embodiments S-BK-5-MAPB Pattern 15B is characterized by any one of embodiments a-o wherein the peaks are within +/−0.3°2theta.
q. In certain embodiments S-BK-5-MAPB Pattern 15B is characterized by any one of embodiments a-o wherein the peaks are within +/−0.2°2theta.
r. In certain embodiments S-BK-5-MAPB Pattern 15B is characterized by any one of embodiments a-q wherein at least three of the recited peaks have a relative peak intensity of at least 10%.
s. In certain embodiments S-BK-5-MAPB Pattern 15B is characterized by any one of embodiments a-q wherein at least three of the recited peaks have a relative peak intensity of at
t. In certain embodiments S-BK-5-MAPB Pattern 15B is characterized by any one of embodiments a-q wherein at least four of the recited peaks have a relative peak intensity of at least 10%.
u. In certain embodiments S-BK-5-MAPB Pattern 15B is characterized by any one of embodiments a-q wherein at least four of the recited peaks have a relative peak intensity of at least 20%.
v. In certain embodiments S-BK-5-MAPB Pattern 15B is characterized by any one of embodiments a-q wherein at least five of the recited peaks have a relative peak intensity of at least 10%.
w. In certain embodiments S-BK-5-MAPB Pattern 15B is characterized by any one of embodiments a-q wherein at least six of the recited peaks have a relative peak intensity of at least 20%.

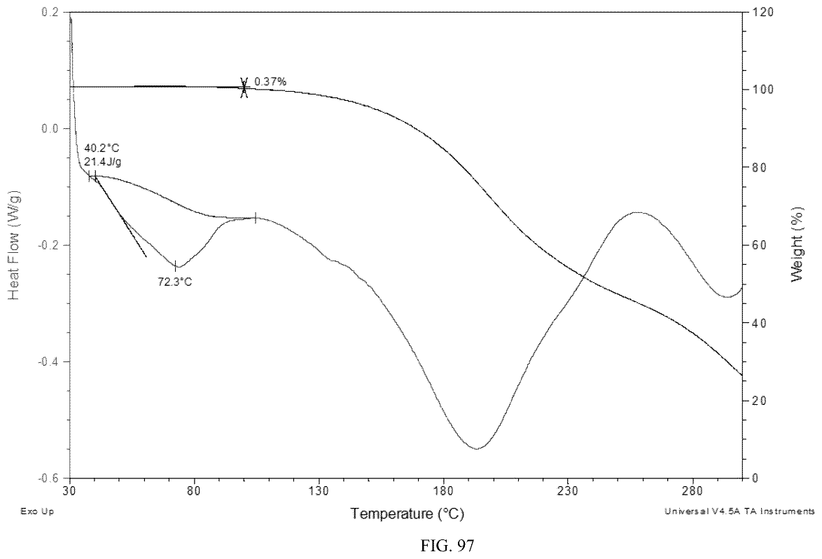

In certain embodiments S-BK-5-MAPB Pattern 15B is characterized by the XRPD diffractogram in FIG. 71 and/or the DSC graph shown in FIG. 97.

### Morphic Forms of S-6-MBPB

### HCl Salt

In certain aspects the invention provides S-6-MBPB as HCl salt. In certain embodiments the S-6-MBPB salt is a stable morphic form denoted Pattern 1A.
a. In certain embodiments S-6-MBPB Pattern 1A is characterized by an XRPD pattern with three or more peaks selected from 14.0, 15.6, 17.8, 18.4, 20.6, 21.0, 22.2, 24.0, 24.8, 25.0, 27.8, 28.1, and 28.3+/−0.4° 2theta.
b. In certain embodiments S-6-MBPB Pattern 1A is characterized by an XRPD pattern with four or more peaks selected from 14.0, 15.6, 17.8, 18.4, 20.6, 21.0, 22.2, 24.0, 24.8, 25.0, 27.8, 28.1, and 28.3+/−0.4° 2theta.
c. In certain embodiments S-6-MBPB Pattern 1A is characterized by an XRPD pattern with five or more peaks selected from 14.0, 15.6, 17.8, 18.4, 20.6, 21.0, 22.2, 24.0, 24.8, 25.0, 27.8, 28.1, and 28.3+/−0.4° 2theta.
d. In certain embodiments S-6-MBPB Pattern 1A is characterized by an XRPD pattern with six or more peaks selected from 14.0, 15.6, 17.8, 18.4, 20.6, 21.0, 22.2, 24.0, 24.8, 25.0, 27.8, 28.1, and 28.3+/−0.4° 2theta.
e. In certain embodiments S-6-MBPB Pattern 1A is characterized by an XRPD pattern with seven or more peaks selected from 14.0, 15.6, 17.8, 18.4, 20.6, 21.0, 22.2, 24.0, 24.8, 25.0, 27.8, 28.1, and 28.3+/−0.4° 2theta.
f. In certain embodiments S-6-MBPB Pattern 1A is characterized by any one of embodiments a-e wherein the XRPD pattern includes a peak at 14.0+/−0.4° 2theta.
g. In certain embodiments S-6-MBPB Pattern 1A is characterized by any one of embodiments a-f wherein the XRPD pattern includes a peak at 15.6+/−0.4°2theta.
h. In certain embodiments S-6-MBPB Pattern 1A is characterized by any one of embodiments a-g wherein the XRPD pattern includes a peak at 17.8+/−0.4° 2theta.
i. In certain embodiments S-6-MBPB Pattern 1A is characterized by any one of embodiments a-h wherein the XRPD pattern includes a peak at 18.4+/−0.4°2theta.
j. In certain embodiments S-6-MBPB Pattern 1A is characterized by any one of embodiments a-i wherein the XRPD pattern includes a peak at 20.6+/−0.4°2theta.
k. In certain embodiments S-6-MBPB Pattern 1A is characterized by any one of embodiments a-i wherein the XRPD pattern includes a peak at 22.2+/−0.4°2theta.
l. In certain embodiments S-6-MBPB Pattern 1A is characterized by any one of embodiments a-i wherein the XRPD pattern includes a peak at 24.8+/−0.4°2theta.
m. In certain embodiments S-6-MBPB Pattern 1A is characterized by any one of embodiments a-i wherein the XRPD pattern includes a peak at 25.0+/−0.4°2theta.
n. In certain embodiments S-6-MBPB Pattern 1A is characterized by any one of embodiments a-i wherein the XRPD pattern includes a peak at 27.8+/−0.4°2theta.
o. In certain embodiments S-6-MBPB Pattern 1A is characterized by any one of embodiments a-i wherein the XRPD pattern includes a peak at 28.1+/−0.4°2theta.
p. In certain embodiments S-6-MBPB Pattern 1A is characterized by any one of embodiments a-o wherein the peaks are within +/−0.3°2theta.
q. In certain embodiments S-6-MBPB Pattern 1A is characterized by any one of embodiments a-o wherein the peaks are within +/−0.2° 2theta.
r. In certain embodiments S-6-MBPB Pattern 1A is characterized by any one of embodiments a-q wherein at least three of the recited peaks have a relative peak intensity of at least 10%.
s. In certain embodiments S-6-MBPB Pattern 1A is characterized by any one of embodiments a-q wherein at least three of the recited peaks have a relative peak intensity of at least 20%.
t. In certain embodiments S-6-MBPB Pattern 1A is characterized by any one of embodiments a-q wherein at least four of the recited peaks have a relative peak intensity of at least 10%.
u. In certain embodiments S-6-MBPB Pattern 1A is characterized by any one of embodiments a-q wherein at least four of the recited peaks have a relative peak intensity of at least 20%.
v. In certain embodiments S-6-MBPB Pattern 1A is characterized by any one of embodiments a-q wherein at least five of the recited peaks have a relative peak intensity of at least 10%.
w. In certain embodiments S-6-MBPB Pattern 1A is characterized by any one of embodiments a-q wherein at least six of the recited peaks have a relative peak intensity of at least 20%.

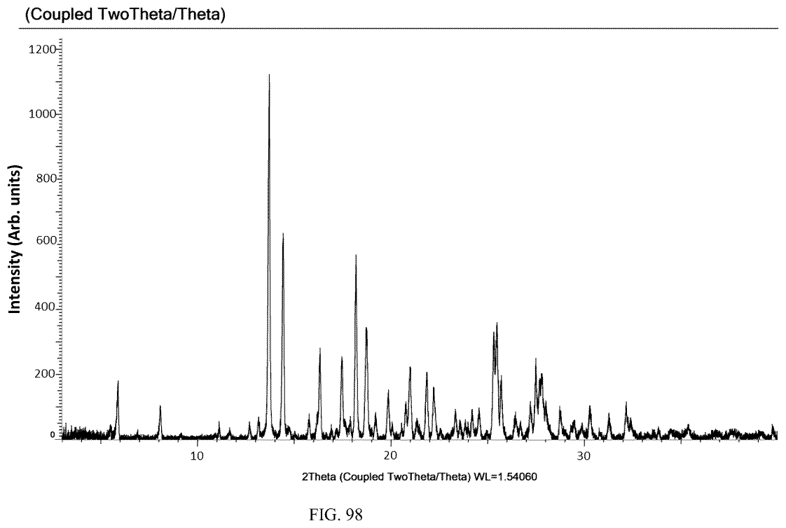

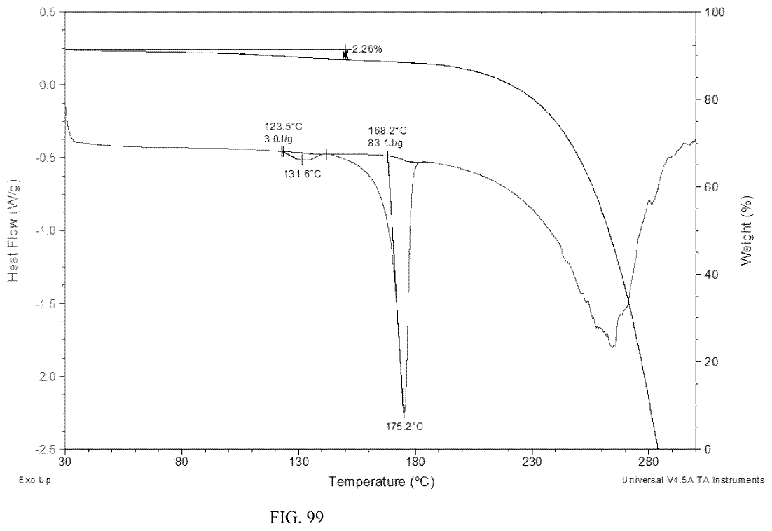

In certain embodiments S-6-MBPB Pattern 1A is characterized by the XRPD diffractogram in FIG. 98 and/or the DSC graph shown in FIG. 99.

### HBr Salt

In certain aspects the invention provides S-6-MBPB as HBr salt. In certain embodiments the S-6-MBPB salt is a stable morphic form denoted Pattern 2A.
a. In certain embodiments S-6-MBPB Pattern 2A is characterized by an XRPD pattern with three or more peaks selected from 7.2, 14.3, 15.7, 18.4, 21.5, 24.3, 24.6, 27.2, and 28.7+/−0.4°2theta.
b. In certain embodiments S-6-MBPB Pattern 2A is characterized by an XRPD pattern with four or more peaks selected from 7.2, 14.3, 15.7, 18.4, 21.5, 24.3, 24.6, 27.2, and 28.7+/−0.4 02theta.
c. In certain embodiments S-6-MBPB Pattern 2A is characterized by an XRPD pattern with five or more peaks selected from 7.2, 14.3, 15.7, 18.4, 21.5, 24.3, 24.6, 27.2, and 28.7+/−0.4°2theta.
d. In certain embodiments S-6-MBPB Pattern 2A is characterized by an XRPD pattern with six or more peaks selected from 7.2, 14.3, 15.7, 18.4, 21.5, 24.3, 24.6, 27.2, and 28.7+/−0.4°2theta.
e. In certain embodiments S-6-MBPB Pattern 2A is characterized by an XRPD pattern with seven or more peaks selected from 7.2, 14.3, 15.7, 18.4, 21.5, 24.3, 24.6, 27.2, and 28.7+/−0.4° 2theta.
f. In certain embodiments S-6-MBPB Pattern 2A is characterized by any one of embodiments a-e wherein the XRPD pattern includes a peak at 7.2+/−0.4° 2theta.
g. In certain embodiments S-6-MBPB Pattern 2A is characterized by any one of embodiments a-f wherein the XRPD pattern includes a peak at 14.3+/−0.4° 2theta.
h. In certain embodiments S-6-MBPB Pattern 2A is characterized by any one of embodiments a-g wherein the XRPD pattern includes a peak at 15.7+/−0.4°2theta.
i. In certain embodiments S-6-MBPB Pattern 2A is characterized by any one of embodiments a-h wherein the XRPD pattern includes a peak at 18.4+/−0.4° 2theta.
j. In certain embodiments S-6-MBPB Pattern 2A is characterized by any one of embodiments a-i wherein the XRPD pattern includes a peak at 21.5+/−0.4°2theta.
k. In certain embodiments S-6-MBPB Pattern 2A is characterized by any one of embodiments a-i wherein the XRPD pattern includes a peak at 24.3+/−0.4°2theta.
l. In certain embodiments S-6-MBPB Pattern 2A is characterized by any one of embodiments a-i wherein the XRPD pattern includes a peak at 24.6+/−0.4°2theta.
m. In certain embodiments S-6-MBPB Pattern 2A is characterized by any one of embodiments a-i wherein the XRPD pattern includes a peak at 27.2+/−0.4°2theta.
n. In certain embodiments S-6-MBPB Pattern 2A is characterized by any one of embodiments a-i wherein the XRPD pattern includes a peak at 27.8+/−0.4°2theta.
o. In certain embodiments S-6-MBPB Pattern 2A is characterized by any one of embodiments a-i wherein the XRPD pattern includes a peak at 28.7+/−0.4°2theta.
p. In certain embodiments S-6-MBPB Pattern 2A is characterized by any one of embodiments a-o wherein the peaks are within +/−0.3°2theta.
q. In certain embodiments S-6-MBPB Pattern 2A is characterized by any one of embodiments a-o wherein the peaks are within +/−0.2° 2theta.
r. In certain embodiments S-6-MBPB Pattern 2A is characterized by any one of embodiments a-q wherein at least three of the recited peaks have a relative peak intensity of at least 10%.
s. In certain embodiments S-6-MBPB Pattern 2A is characterized by any one of embodiments a-q wherein at least three of the recited peaks have a relative peak intensity of at least 20%.
t. In certain embodiments S-6-MBPB Pattern 2A is characterized by any one of embodiments a-q wherein at least four of the recited peaks have a relative peak intensity of at least 10%.
u. In certain embodiments S-6-MBPB Pattern 2A is characterized by any one of embodiments a-q wherein at least four of the recited peaks have a relative peak intensity of at least 20%.
v. In certain embodiments S-6-MBPB Pattern 2A is characterized by any one of embodiments a-q wherein at least five of the recited peaks have a relative peak intensity of at least 10%.
w. In certain embodiments S-6-MBPB Pattern 2A is characterized by any one of embodiments a-q wherein at least six of the recited peaks have a relative peak intensity of at least 20%.

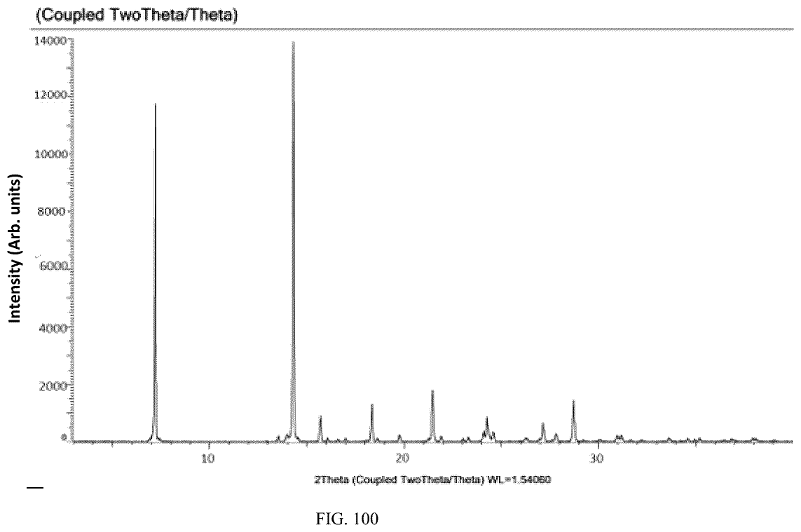

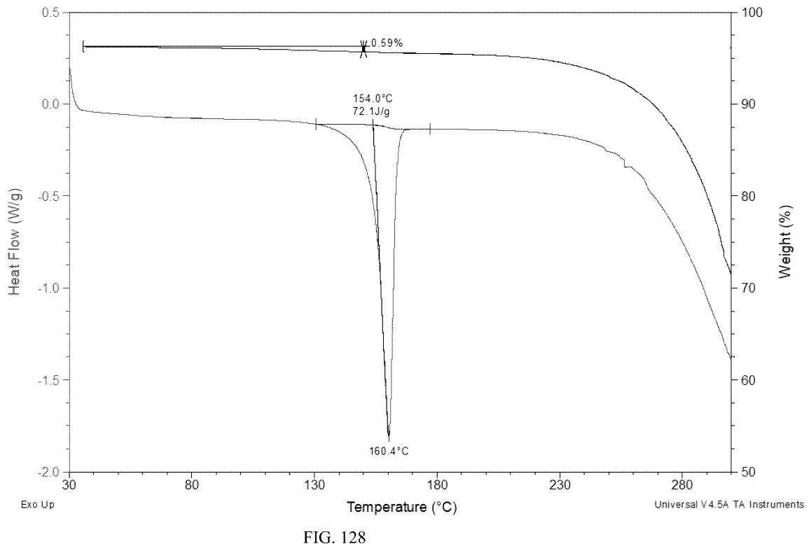

In certain embodiments S-6-MBPB Pattern 2A is characterized by the XRPD diffractogram in FIG. 100 and/or the DSC graph shown in FIG. 128.

H₃PO₄ Salt

In certain aspects the invention provides S-6-MBPB as H₃PO₄ salt. In certain embodiments the S-6-MBPB salt is a stable morphic form denoted Pattern 4A.
a. In certain embodiments S-6-MBPB Pattern 4A is characterized by an XRPD pattern with three or more peaks selected from 5.6, 9.1, 11.1, 13.2, 13.9, 14.3, 14.6, 15.4, 15.6, 15.9, 17.5, 18.1, 20.0, 20.3, 21.5, 23.1, 25.1, 26.9, and 28.0+/−0.4° 2theta.
b. In certain embodiments S-6-MBPB Pattern 4A is characterized by an XRPD pattern with four or more peaks selected from 5.6, 9.1, 11.1, 13.2, 13.9, 14.3, 14.6, 15.4, 15.6, 15.9, 17.5, 18.1, 20.0, 20.3, 21.5, 23.1, 25.1, 26.9, and 28.0+/−0.4° 2theta.
c. In certain embodiments S-6-MBPB Pattern 4A is characterized by an XRPD pattern with five or more peaks selected from 5.6, 9.1, 11.1, 13.2, 13.9, 14.3, 14.6, 15.4, 15.6, 15.9, 17.5, 18.1, 20.0, 20.3, 21.5, 23.1, 25.1, 26.9, and 28.0+/−0.4°2theta.
d. In certain embodiments S-6-MBPB Pattern 4A is characterized by an XRPD pattern with six or more peaks selected from 5.6, 9.1, 11.1, 13.2, 13.9, 14.3, 14.6, 15.4, 15.6, 15.9, 17.5, 18.1, 20.0, 20.3, 21.5, 23.1, 25.1, 26.9, and 28.0+/−0.4°2theta.
e. In certain embodiments S-6-MBPB Pattern 4A is characterized by an XRPD pattern with seven or more peaks selected from 5.6, 9.1, 11.1, 13.2, 13.9, 14.3, 14.6, 15.4, 15.6, 15.9, 17.5, 18.1, 20.0, 20.3, 21.5, 23.1, 25.1, 26.9, and 28.0+/−0.4° 2theta.
f. In certain embodiments S-6-MBPB Pattern 4A is characterized by any one of embodiments a-e wherein the XRPD pattern includes a peak at 5.6+/−0.4° 2theta.
g. In certain embodiments S-6-MBPB Pattern 4A is characterized by any one of embodiments a-f wherein the XRPD pattern includes a peak at 13.9+/−0.4°2theta.
h. In certain embodiments S-6-MBPB Pattern 4A is characterized by any one of embodiments a-g wherein the XRPD pattern includes a peak at 15.4+/−0.4° 2theta.
i. In certain embodiments S-6-MBPB Pattern 4A is characterized by any one of embodiments a-h wherein the XRPD pattern includes a peak at 15.9+/−0.4°2theta.
j. In certain embodiments S-6-MBPB Pattern 4A is characterized by any one of embodiments a-i wherein the XRPD pattern includes a peak at 17.5+/−0.4°2theta.
k. In certain embodiments S-6-MBPB Pattern 4A is characterized by any one of embodiments a-i wherein the XRPD pattern includes a peak at 18.1+/−0.4°2theta.
l. In certain embodiments S-6-MBPB Pattern 4A is characterized by any one of embodiments a-i wherein the XRPD pattern includes a peak at 20.0+/−0.4°2theta.
m. In certain embodiments S-6-MBPB Pattern 4A is characterized by any one of embodiments a-i wherein the XRPD pattern includes a peak at 20.3+/−0.4°2theta.
n. In certain embodiments S-6-MBPB Pattern 4A is characterized by any one of embodiments a-i wherein the XRPD pattern includes a peak at 21.5+/−0.4° 2theta.
o. In certain embodiments S-6-MBPB Pattern 4A is characterized by any one of embodiments a-i wherein the XRPD pattern includes a peak at 23.1+/−0.4°2theta.
p. In certain embodiments S-6-MBPB Pattern 4A is characterized by any one of embodiments a-o wherein the peaks are within +/−0.3°2theta.
q. In certain embodiments S-6-MBPB Pattern 4A is characterized by any one of embodiments a-o wherein the peaks are within +/−0.2°2theta.
r. In certain embodiments S-6-MBPB Pattern 4A is characterized by any one of embodiments a-q wherein at least three of the recited peaks have a relative peak intensity of at least 10%.
s. In certain embodiments S-6-MBPB Pattern 4A is characterized by any one of embodiments a-q wherein at least three of the recited peaks have a relative peak intensity of at least 20%.
t. In certain embodiments S-6-MBPB Pattern 4A is characterized by any one of embodiments a-q wherein at least four of the recited peaks have a relative peak intensity of at least 10%.
u. In certain embodiments S-6-MBPB Pattern 4A is characterized by any one of embodiments a-q wherein at least four of the recited peaks have a relative peak intensity of at least 20%.
v. In certain embodiments S-6-MBPB Pattern 4A is characterized by any one of embodiments a-q wherein at least five of the recited peaks have a relative peak intensity of at least 10%.
w. In certain embodiments S-6-MBPB Pattern 4A is characterized by any one of embodiments a-q wherein at least six of the recited peaks have a relative peak intensity of at least 20%.

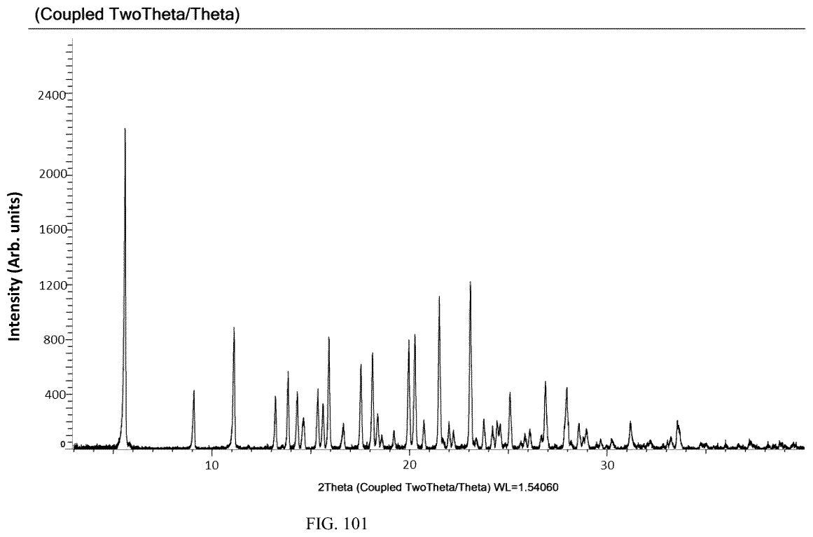

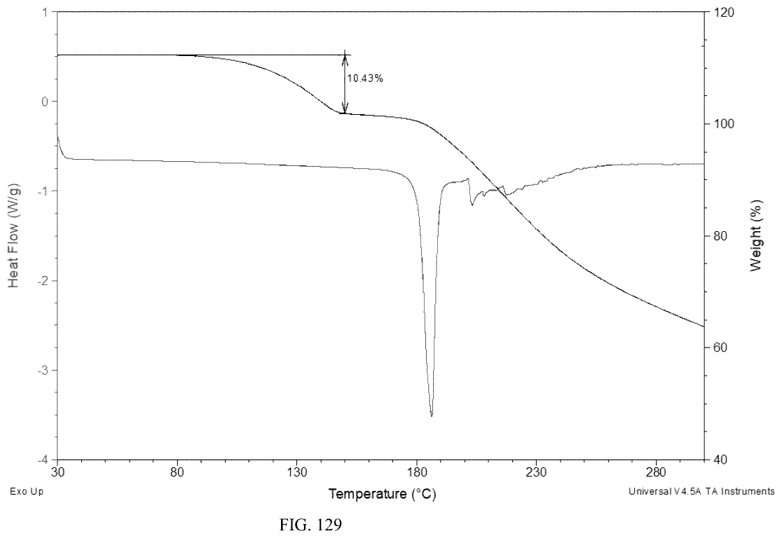

In certain embodiments S-6-MBPB Pattern 4A is characterized by the XRPD diffractogram in FIG. 101 and/or the DSC graph shown in FIG. 129.

### HNO₃ Salt

In certain aspects the invention provides S-6-MBPB as HNO₃ salt. In certain embodiments the S-6-MBPB salt is a stable morphic form denoted Pattern 5A.
a. In certain embodiments S-6-MBPB Pattern 5A is characterized by an XRPD pattern with three or more peaks selected from 14.6, 14.7, 16.6, 17.7, 19.1, 19.9, 22.2, 23.4, 23.7, 14.9, 25.9, 17.5, 29.2, and 30.7+/−0.4° 2theta.
b. In certain embodiments S-6-MBPB Pattern 5A is characterized by an XRPD pattern with four or more peaks selected from 14.6, 14.7, 16.6, 17.7, 19.1, 19.9, 22.2, 23.4, 23.7, 14.9, 25.9, 17.5, 29.2, and 30.7+/−0.4° 2theta.
c. In certain embodiments S-6-MBPB Pattern 5A is characterized by an XRPD pattern with five or more peaks selected from 14.6, 14.7, 16.6, 17.7, 19.1, 19.9, 22.2, 23.4, 23.7, 14.9, 25.9, 17.5, 29.2, and 30.7+/−0.4°2theta.
d. In certain embodiments S-6-MBPB Pattern 5A is characterized by an XRPD pattern with six or more peaks selected from 14.6, 14.7, 16.6, 17.7, 19.1, 19.9, 22.2, 23.4, 23.7, 14.9, 25.9, 17.5, 29.2, and 30.7+/−0.4° 2theta.
e. In certain embodiments S-6-MBPB Pattern 5A is characterized by an XRPD pattern with seven or more peaks selected from 14.6, 14.7, 16.6, 17.7, 19.1, 19.9, 22.2, 23.4, 23.7, 14.9, 25.9, 17.5, 29.2, and 30.7+/−0.4° 2theta.
f. In certain embodiments S-6-MBPB Pattern 5A is characterized by any one of embodiments a-e wherein the XRPD pattern includes a peak at 14.6+/−0.4° 2theta.
g. In certain embodiments S-6-MBPB Pattern 5A is characterized by any one of embodiments a-f wherein the XRPD pattern includes a peak at 14.7+/−0.4° 2theta.
h. In certain embodiments S-6-MBPB Pattern 5A is characterized by any one of embodiments a-g wherein the XRPD pattern includes a peak at 16.6+/−0.4°2theta.
i. In certain embodiments S-6-MBPB Pattern 5A is characterized by any one of embodiments a-h wherein the XRPD pattern includes a peak at 17.7+/−0.4°2theta.
j. In certain embodiments S-6-MBPB Pattern 5A is characterized by any one of embodiments a-i wherein the XRPD pattern includes a peak at 19.1+/−0.4° 2theta.
k. In certain embodiments S-6-MBPB Pattern 5A is characterized by any one of embodiments a-i wherein the XRPD pattern includes a peak at 19.9+/−0.4°2theta.
l. In certain embodiments S-6-MBPB Pattern 5A is characterized by any one of embodiments a-i wherein the XRPD pattern includes a peak at 23.4+/−0.4°2theta.
m. In certain embodiments S-6-MBPB Pattern 5A is characterized by any one of embodiments a-i wherein the XRPD pattern includes a peak at 23.7+/−0.4°2theta.
n. In certain embodiments S-6-MBPB Pattern 5A is characterized by any one of embodiments a-i wherein the XRPD pattern includes a peak at 24.9+/−0.4°2theta.
o. In certain embodiments S-6-MBPB Pattern 5A is characterized by any one of embodiments a-i wherein the XRPD pattern includes a peak at 25.9+/−0.4°2theta.
p. In certain embodiments S-6-MBPB Pattern 5A is characterized by any one of embodiments a-o wherein the peaks are within +/−0.3°2theta.
q. In certain embodiments S-6-MBPB Pattern 5A is characterized by any one of embodiments a-o wherein the peaks are within +/−0.2°2theta.
r. In certain embodiments S-6-MBPB Pattern 5A is characterized by any one of embodiments a-q wherein at least three of the recited peaks have a relative peak intensity of at least 10%.
s. In certain embodiments S-6-MBPB Pattern 5A is characterized by any one of embodiments a-q wherein at least three of the recited peaks have a relative peak intensity of at least 20%.
t. In certain embodiments S-6-MBPB Pattern 5A is characterized by any one of embodiments a-q wherein at least four of the recited peaks have a relative peak intensity of at least 10%.
u. In certain embodiments S-6-MBPB Pattern 5A is characterized by any one of embodiments a-q wherein at least four of the recited peaks have a relative peak intensity of at least 20%.
v. In certain embodiments S-6-MBPB Pattern 5A is characterized by any one of embodiments a-q wherein at least five of the recited peaks have a relative peak intensity of at least 10%.
w. In certain embodiments S-6-MBPB Pattern 5A is characterized by any one of embodiments a-q wherein at least six of the recited peaks have a relative peak intensity of at least 20%.

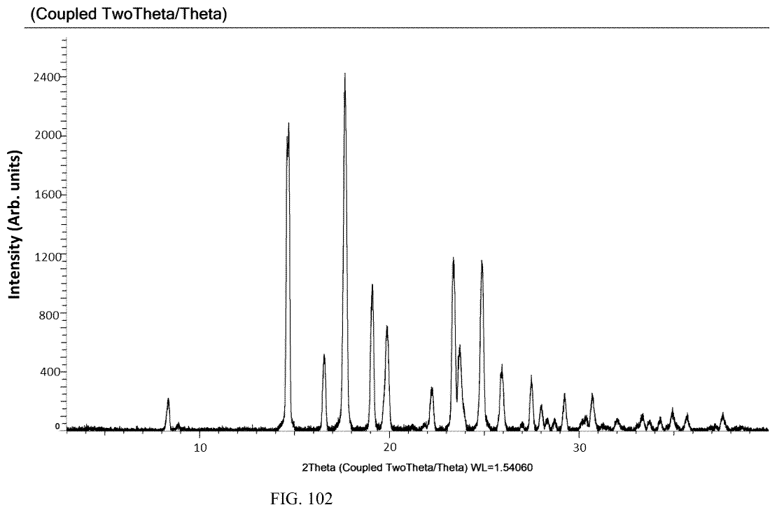

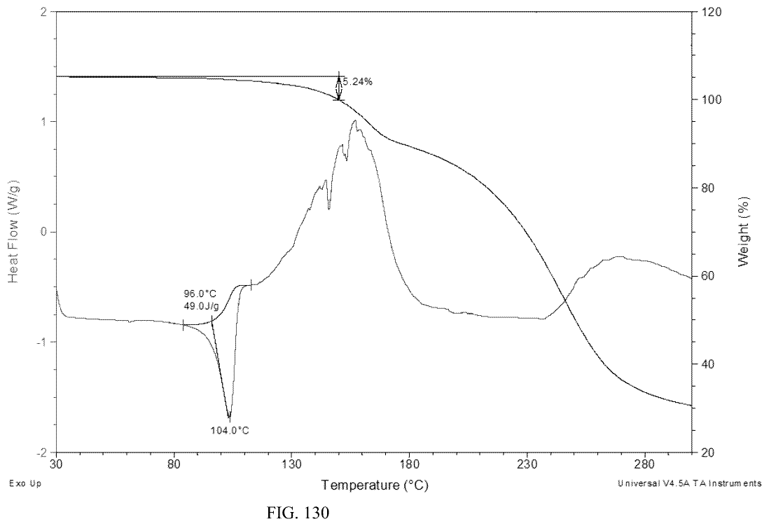

In certain embodiments S-6-MBPB Pattern 5A is characterized by the XRPD diffractogram in FIG. 102 and/or the DSC graph shown in FIG. 130.

### Tartaric Salt

In certain aspects the invention provides S-6-MBPB as tartaric salt. In certain embodiments the S-6-MBPB salt is a stable morphic form denoted Pattern 7A.
a. In certain embodiments S-6-MBPB Pattern 7A is characterized by an XRPD pattern with three or more peaks selected from 6.4, 12.2, 12.6, 14.8, 16.4, 16.6, 16.8, 17.5, 17.9, 18.3, 19.4, 20.0, 20.6, 22.1, 23.2, 23.9, 24.5, 25.3, 25.8, 27.0, 28.5, and 32.0+/−0.4°2theta.
b. In certain embodiments S-6-MBPB Pattern 7A is characterized by an XRPD pattern with four or more peaks selected from 6.4, 12.2, 12.6, 14.8, 16.4, 16.6, 16.8, 17.5, 17.9, 18.3, 19.4, 20.0, 20.6, 22.1, 23.2, 23.9, 24.5, 25.3, 25.8, 27.0, 28.5, and 32.0+/−0.4°2theta.
c. In certain embodiments S-6-MBPB Pattern 7A is characterized by an XRPD pattern with five or more peaks selected from 6.4, 12.2, 12.6, 14.8, 16.4, 16.6, 16.8, 17.5, 17.9, 18.3, 19.4, 20.0, 20.6, 22.1, 23.2, 23.9, 24.5, 25.3, 25.8, 27.0, 28.5, and 32.0+/−0.4°2theta.
d. In certain embodiments S-6-MBPB Pattern 7A is characterized by an XRPD pattern with six or more peaks selected from 6.4, 12.2, 12.6, 14.8, 16.4, 16.6, 16.8, 17.5, 17.9, 18.3, 19.4, 20.0, 20.6, 22.1, 23.2, 23.9, 24.5, 25.3, 25.8, 27.0, 28.5, and 32.0+/−0.4°2theta.
e. In certain embodiments S-6-MBPB Pattern 7A is characterized by an XRPD pattern with seven or more peaks selected from 6.4, 12.2, 12.6, 14.8, 16.4, 16.6, 16.8, 17.5, 17.9, 18.3, 19.4, 20.0, 20.6, 22.1, 23.2, 23.9, 24.5, 25.3, 25.8, 27.0, 28.5, and 32.0+/−0.4° 2theta.
f. In certain embodiments S-6-MBPB Pattern 7A is characterized by any one of embodiments a-e wherein the XRPD pattern includes a peak at 6.4+/−0.4° 2theta.
g. In certain embodiments S-6-MBPB Pattern 7A is characterized by any one of embodiments a-f wherein the XRPD pattern includes a peak at 12.6+/−0.4°2theta.
h. In certain embodiments S-6-MBPB Pattern 7A is characterized by any one of embodiments a-g wherein the XRPD pattern includes a peak at 14.8+/−0.4°2theta.
i. In certain embodiments S-6-MBPB Pattern 7A is characterized by any one of embodiments a-h wherein the XRPD pattern includes a peak at 16.4+/−0.4° 2theta.
j. In certain embodiments S-6-MBPB Pattern 7A is characterized by any one of embodiments a-i wherein the XRPD pattern includes a peak at 16.6+/−0.4° 2theta.
k. In certain embodiments S-6-MBPB Pattern 7A is characterized by any one of embodiments a-i wherein the XRPD pattern includes a peak at 17.5+/−0.4°2theta.
l. In certain embodiments S-6-MBPB Pattern 7A is characterized by any one of embodiments a-i wherein the XRPD pattern includes a peak at 17.9+/−0.4°2theta.
m. In certain embodiments S-6-MBPB Pattern 7A is characterized by any one of embodiments a-i wherein the XRPD pattern includes a peak at 18.3+/−0.4° 2theta.
n. In certain embodiments S-6-MBPB Pattern 7A is characterized by any one of embodiments a-i wherein the XRPD pattern includes a peak at 19.4+/−0.4° 2theta.
o. In certain embodiments S-6-MBPB Pattern 7A is characterized by any one of embodiments a-i wherein the XRPD pattern includes a peak at 22.1+/−0.4°2theta.
p. In certain embodiments S-6-MBPB Pattern 7A is characterized by any one of embodiments a-o wherein the peaks are within +/−0.3°2theta.
q. In certain embodiments S-6-MBPB Pattern 7A is characterized by any one of embodiments a-o wherein the peaks are within +/−0.2° 2theta.
r. In certain embodiments S-6-MBPB Pattern 7A is characterized by any one of embodiments a-q wherein at least three of the recited peaks have a relative peak intensity of at least 10%.
s. In certain embodiments S-6-MBPB Pattern 7A is characterized by any one of embodiments a-q wherein at least three of the recited peaks have a relative peak intensity of at least 20%.
t. In certain embodiments S-6-MBPB Pattern 7A is characterized by any one of embodiments a-q wherein at least four of the recited peaks have a relative peak intensity of at least 10%.
u. In certain embodiments S-6-MBPB Pattern 7A is characterized by any one of embodiments a-q wherein at least four of the recited peaks have a relative peak intensity of at least 20%.
v. In certain embodiments S-6-MBPB Pattern 7A is characterized by any one of embodiments a-q wherein at least five of the recited peaks have a relative peak intensity of at least 10%.
w. In certain embodiments S-6-MBPB Pattern 7A is characterized by any one of embodiments a-q wherein at least six of the recited peaks have a relative peak intensity of at least 20%.

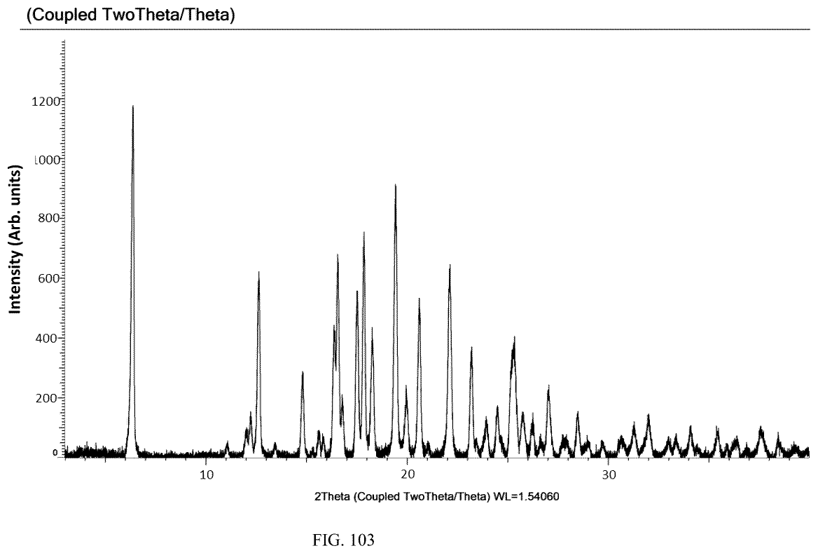

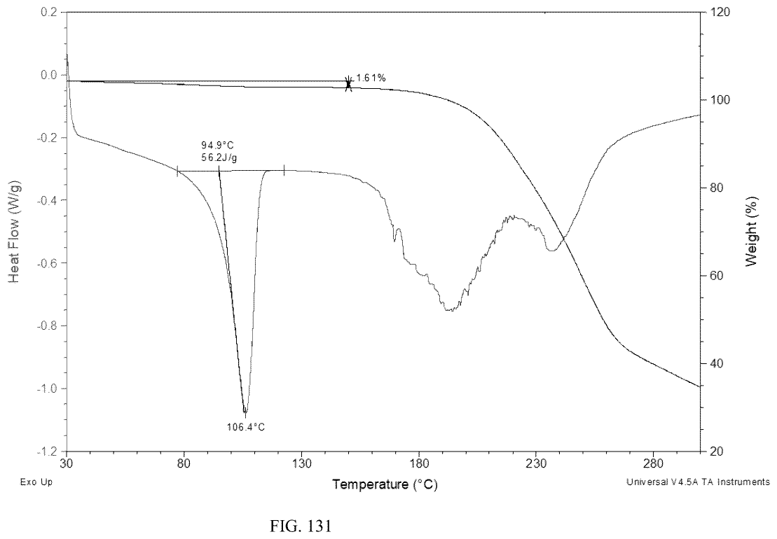

In certain embodiments S-6-MBPB Pattern 7A is characterized by the XRPD diffractogram in FIG. 103 and/or the DSC graph shown in FIG. 131.

### Succinic Salt

In certain aspects the invention provides S-6-MBPB as succinic salt. In certain embodiments the S-6-MBPB salt is a stable morphic form denoted Pattern 8A.
a. In certain embodiments S-6-MBPB Pattern 8A is characterized by an XRPD pattern with three or more peaks selected from 12.0, 12.2, 12.7, 12.9, 14.8, 17.1, 17.6, 19.1, 20.0, 20.3, 21.7, 22.1, 22.3, 22.8, 23.1, 24.0, 24.4, 24.8, 25.5, 25.9, 26.6, 26.9, 27.3, 27.9, 28.7, and 33.1+/−0.4°2theta.
b. In certain embodiments S-6-MBPB Pattern 8A is characterized by an XRPD pattern with four or more peaks selected from 12.0, 12.2, 12.7, 12.9, 14.8, 17.1, 17.6, 19.1, 20.0, 20.3, 21.7, 22.1, 22.3, 22.8, 23.1, 24.0, 24.4, 24.8, 25.5, 25.9, 26.6, 26.9, 27.3, 27.9, 28.7, and 33.1+/−0.4° 2theta.
c. In certain embodiments S-6-MBPB Pattern 8A is characterized by an XRPD pattern with five or more peaks selected from 12.0, 12.2, 12.7, 12.9, 14.8, 17.1, 17.6, 19.1, 20.0, 20.3, 21.7, 22.1, 22.3, 22.8, 23.1, 24.0, 24.4, 24.8, 25.5, 25.9, 26.6, 26.9, 27.3, 27.9, 28.7, and 33.1+/−0.4° 2theta.
d. In certain embodiments S-6-MBPB Pattern 8A is characterized by an XRPD pattern with six or more peaks selected from 12.0, 12.2, 12.7, 12.9, 14.8, 17.1, 17.6, 19.1, 20.0, 20.3, 21.7, 22.1, 22.3, 22.8, 23.1, 24.0, 24.4, 24.8, 25.5, 25.9, 26.6, 26.9, 27.3, 27.9, 28.7, and 33.1+/−0.4°2theta.
e. In certain embodiments S-6-MBPB Pattern 8A is characterized by an XRPD pattern with seven or more peaks selected from 12.0, 12.2, 12.7, 12.9, 14.8, 17.1, 17.6, 19.1, 20.0, 20.3, 21.7, 22.1, 22.3, 22.8, 23.1, 24.0, 24.4, 24.8, 25.5, 25.9, 26.6, 26.9, 27.3, 27.9, 28.7, and 33.1+/−0.4° 2theta.
f. In certain embodiments S-6-MBPB Pattern 8A is characterized by any one of embodiments a-e wherein the XRPD pattern includes a peak at 12.0+/−0.4° 2theta.
g. In certain embodiments S-6-MBPB Pattern 8A is characterized by any one of embodiments a-f wherein the XRPD pattern includes a peak at 12.2+/−0.4°2theta.
h. In certain embodiments S-6-MBPB Pattern 8A is characterized by any one of embodiments a-g wherein the XRPD pattern includes a peak at 12.9+/−0.4°2theta.
i. In certain embodiments S-6-MBPB Pattern 8A is characterized by any one of embodiments a-h wherein the XRPD pattern includes a peak at 17.1+/−0.4°2theta.
j. In certain embodiments S-6-MBPB Pattern 8A is characterized by any one of embodiments a-i wherein the XRPD pattern includes a peak at 17.6+/−0.4°2theta.
k. In certain embodiments S-6-MBPB Pattern 8A is characterized by any one of embodiments a-i wherein the XRPD pattern includes a peak at 20.3+/−0.4°2theta.
l. In certain embodiments S-6-MBPB Pattern 8A is characterized by any one of embodiments a-i wherein the XRPD pattern includes a peak at 22.1+/−0.4°2theta.
m. In certain embodiments S-6-MBPB Pattern 8A is characterized by any one of embodiments a-i wherein the XRPD pattern includes a peak at 22.3+/−0.4°2theta.
n. In certain embodiments S-6-MBPB Pattern 8A is characterized by any one of embodiments a-i wherein the XRPD pattern includes a peak at 22.8+/−0.4°2theta.
o. In certain embodiments S-6-MBPB Pattern 8A is characterized by any one of embodiments a-i wherein the XRPD pattern includes a peak at 25.5+/−0.4°2theta.
p. In certain embodiments S-6-MBPB Pattern 8A is characterized by any one of embodiments a-o wherein the peaks are within +/−0.3°2theta.
q. In certain embodiments S-6-MBPB Pattern 8A is characterized by any one of embodiments a-o wherein the peaks are within +/−0.2°2theta.
r. In certain embodiments S-6-MBPB Pattern 8A is characterized by any one of embodiments a-q wherein at least three of the recited peaks have a relative peak intensity of at least 10%.
s. In certain embodiments S-6-MBPB Pattern 8A is characterized by any one of embodiments a-q wherein at least three of the recited peaks have a relative peak intensity of at
t. In certain embodiments S-6-MBPB Pattern 8A is characterized by any one of embodiments a-q wherein at least four of the recited peaks have a relative peak intensity of at least 10%.
u. In certain embodiments S-6-MBPB Pattern 8A is characterized by any one of embodiments a-q wherein at least four of the recited peaks have a relative peak intensity of at least 20%.
v. In certain embodiments S-6-MBPB Pattern 8A is characterized by any one of embodiments a-q wherein at least five of the recited peaks have a relative peak intensity of at least 10%.
w. In certain embodiments S-6-MBPB Pattern 8A is characterized by any one of embodiments a-q wherein at least six of the recited peaks have a relative peak intensity of at least 20%.

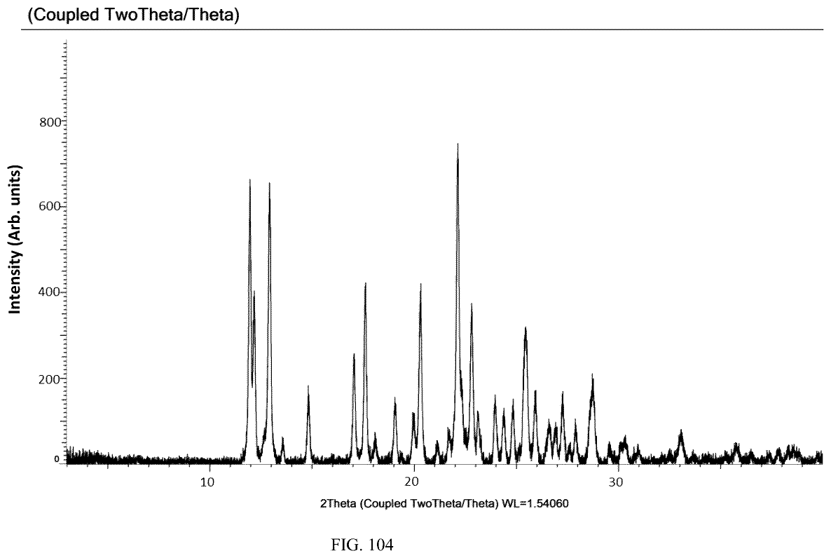

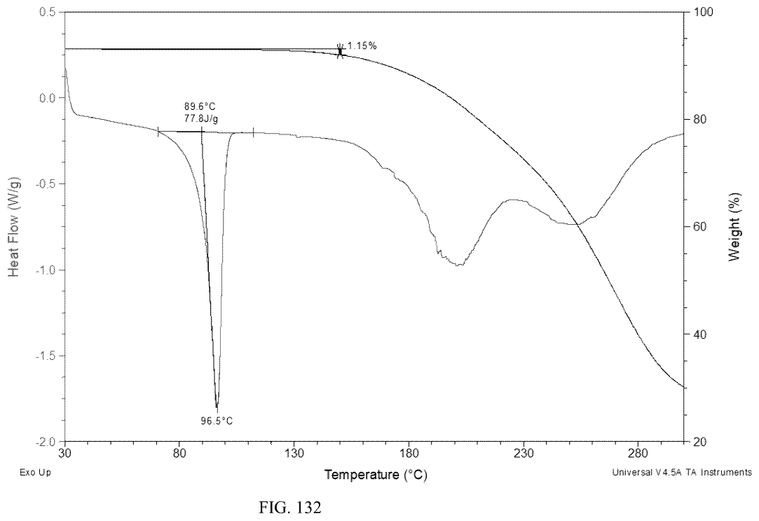

In certain embodiments S-6-MBPB Pattern 8A is characterized by the XRPD diffractogram in FIG. 104 and/or the DSC graph shown in FIG. 132.

### Oxalate Salt

In certain aspects the invention provides S-6-MBPB as oxalate salt. In certain embodiments the S-6-MBPB salt is a stable morphic form denoted Pattern 9A.
a. In certain embodiments S-6-MBPB Pattern 9A is characterized by an XRPD pattern with three or more peaks selected from 11.8, 15.5, 15.9, 17.0, 19.1, 20.0, 20.9, 21.1, 21.7, 25.9, 27.8, and 32.6+/−0.4° 2theta.
b. In certain embodiments S-6-MBPB Pattern 9A is characterized by an XRPD pattern with four or more peaks selected from 11.8, 15.5, 15.9, 17.0, 19.1, 20.0, 20.9, 21.1, 21.7, 25.9, 27.8, and 32.6+/−0.4° 2theta.
c. In certain embodiments S-6-MBPB Pattern 9A is characterized by an XRPD pattern with five or more peaks selected from 11.8, 15.5, 15.9, 17.0, 19.1, 20.0, 20.9, 21.1, 21.7, 25.9, 27.8, and 32.6+/−0.4° 2theta.
d. In certain embodiments S-6-MBPB Pattern 9A is characterized by an XRPD pattern with six or more peaks selected from 11.8, 15.5, 15.9, 17.0, 19.1, 20.0, 20.9, 21.1, 21.7, 25.9, 27.8, and 32.6+/−0.4° 2theta.
e. In certain embodiments S-6-MBPB Pattern 9A is characterized by an XRPD pattern with seven or more peaks selected from 11.8, 15.5, 15.9, 17.0, 19.1, 20.0, 20.9, 21.1, 21.7, 25.9, 27.8, and 32.6+/−0.4° 2theta.
f. In certain embodiments S-6-MBPB Pattern 9A is characterized by any one of embodiments a-e wherein the XRPD pattern includes a peak at 11.8+/−0.4° 2theta.
g. In certain embodiments S-6-MBPB Pattern 9A is characterized by any one of embodiments a-f wherein the XRPD pattern includes a peak at 15.5+/−0.4°2theta.
h. In certain embodiments S-6-MBPB Pattern 9A is characterized by any one of embodiments a-g wherein the XRPD pattern includes a peak at 15.9+/−0.4°2theta.
i. In certain embodiments S-6-MBPB Pattern 9A is characterized by any one of embodiments a-h wherein the XRPD pattern includes a peak at 19.1+/−0.4°2theta.
j. In certain embodiments S-6-MBPB Pattern 9A is characterized by any one of embodiments a-i wherein the XRPD pattern includes a peak at 20.9+/−0.4°2theta.
k. In certain embodiments S-6-MBPB Pattern 9A is characterized by any one of embodiments a-i wherein the XRPD pattern includes a peak at 21.1+/−0.4°2theta.
l. In certain embodiments S-6-MBPB Pattern 9A is characterized by any one of embodiments a-i wherein the XRPD pattern includes a peak at 21.7+/−0.4°2theta.
m. In certain embodiments S-6-MBPB Pattern 9A is characterized by any one of embodiments a-i wherein the XRPD pattern includes a peak at 25.9+/−0.4° 2theta.
n. In certain embodiments S-6-MBPB Pattern 9A is characterized by any one of embodiments a-i wherein the XRPD pattern includes a peak at 27.8+/−0.4°2theta.
o. In certain embodiments S-6-MBPB Pattern 9A is characterized by any one of embodiments a-i wherein the XRPD pattern includes a peak at 32.6+/−0.4° 2theta.
p. In certain embodiments S-6-MBPB Pattern 9A is characterized by any one of embodiments a-o wherein the peaks are within +/−0.3°2theta.
q. In certain embodiments S-6-MBPB Pattern 9A is characterized by any one of embodiments a-o wherein the peaks are within +/−0.2°2theta.
r. In certain embodiments S-6-MBPB Pattern 9A is characterized by any one of embodiments a-q wherein at least three of the recited peaks have a relative peak intensity of at least 10%.
s. In certain embodiments S-6-MBPB Pattern 9A is characterized by any one of embodiments a-q wherein at least three of the recited peaks have a relative peak intensity of at least 20%.
t. In certain embodiments S-6-MBPB Pattern 9A is characterized by any one of embodiments a-q wherein at least four of the recited peaks have a relative peak intensity of at least 10%.
u. In certain embodiments S-6-MBPB Pattern 9A is characterized by any one of embodiments a-q wherein at least four of the recited peaks have a relative peak intensity of at least 20%.
v. In certain embodiments S-6-MBPB Pattern 9A is characterized by any one of embodiments a-q wherein at least five of the recited peaks have a relative peak intensity of at least 10%.
w. In certain embodiments S-6-MBPB Pattern 9A is characterized by any one of embodiments a-q wherein at least six of the recited peaks have a relative peak intensity of at least 20%.

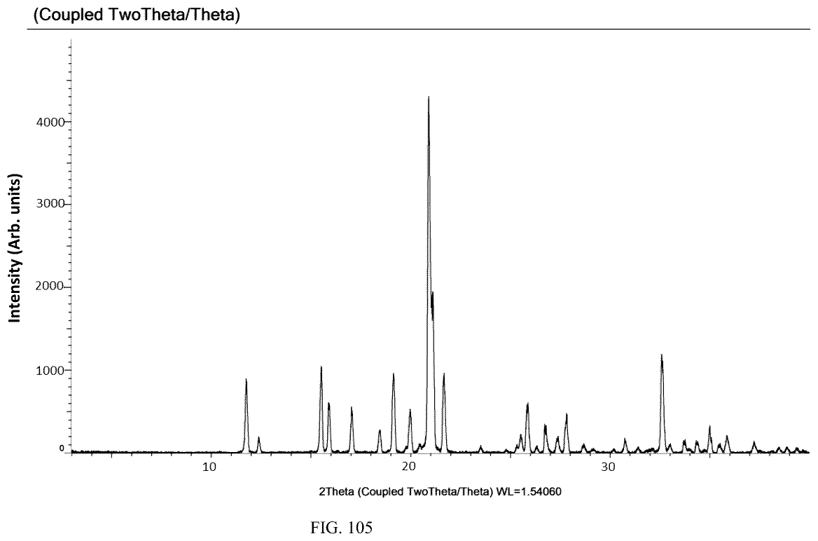

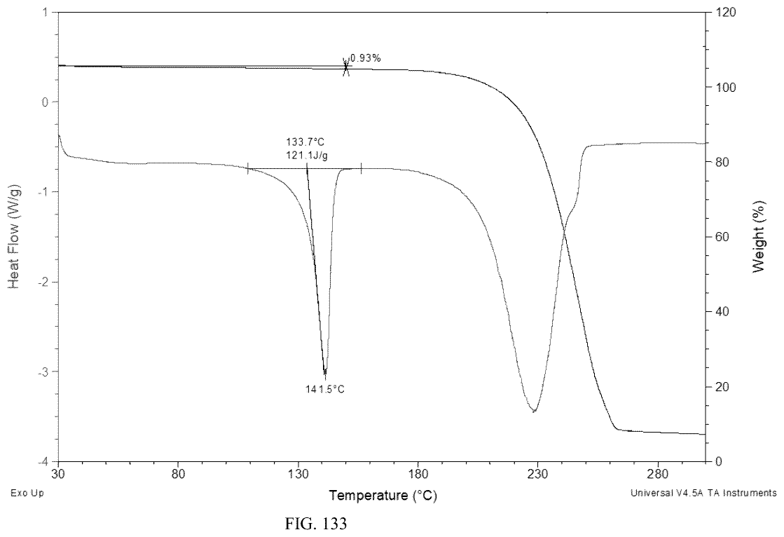

In certain embodiments S-6-MBPB Pattern 9A is characterized by the XRPD diffractogram in FIG. 105 and/or the DSC graph shown in FIG. 133.

### Maleic Salt

In certain aspects the invention provides S-6-MBPB as maleic salt. In certain embodiments the S-6-MBPB salt is a stable morphic form denoted Pattern 10A.
a. In certain embodiments S-6-MBPB Pattern 10A is characterized by an XRPD pattern with three or more peaks selected from 12.7, 13.7, 15.3, 17.9, 19.0, 19.7, 20.0, 20.1, 20.9, 21.6, 21.9, 23.2, 23.4, 23.5, 25.3, 27.2, 27.5, 33.4, and 34.0+/−0.4°2theta.
b. In certain embodiments S-6-MBPB Pattern 10A is characterized by an XRPD pattern with four or more peaks selected from 12.7, 13.7, 15.3, 17.9, 19.0, 19.7, 20.0, 20.1, 20.9, 21.6, 21.9, 23.2, 23.4, 23.5, 25.3, 27.2, 27.5, 33.4, and 34.0+/−0.4°2theta.
c. In certain embodiments S-6-MBPB Pattern 10A is characterized by an XRPD pattern with five or more peaks selected from 12.7, 13.7, 15.3, 17.9, 19.0, 19.7, 20.0, 20.1, 20.9, 21.6, 21.9, 23.2, 23.4, 23.5, 25.3, 27.2, 27.5, 33.4, and 34.0+/−0.4°2theta.
d. In certain embodiments S-6-MBPB Pattern 10A is characterized by an XRPD pattern with six or more peaks selected from 12.7, 13.7, 15.3, 17.9, 19.0, 19.7, 20.0, 20.1, 20.9, 21.6, 21.9, 23.2, 23.4, 23.5, 25.3, 27.2, 27.5, 33.4, and 34.0+/−0.4° 2theta.
e. In certain embodiments S-6-MBPB Pattern 10A is characterized by an XRPD pattern with seven or more peaks selected from 12.7, 13.7, 15.3, 17.9, 19.0, 19.7, 20.0, 20.1, 20.9, 21.6, 21.9, 23.2, 23.4, 23.5, 25.3, 27.2, 27.5, 33.4, and 34.0+/−0.4°2theta.
f. In certain embodiments S-6-MBPB Pattern 10A is characterized by any one of embodiments a-e wherein the XRPD pattern includes a peak at 12.7+/−0.4° 2theta.
g. In certain embodiments S-6-MBPB Pattern 10A is characterized by any one of embodiments a-f wherein the XRPD pattern includes a peak at 13.7+/−0.4°2theta.
h. In certain embodiments S-6-MBPB Pattern 10A is characterized by any one of embodiments a-g wherein the XRPD pattern includes a peak at 15.3+/−0.4°2theta.
i. In certain embodiments S-6-MBPB Pattern 10A is characterized by any one of embodiments a-h wherein the XRPD pattern includes a peak at 17.9+/−0.4°2theta.
j. In certain embodiments S-6-MBPB Pattern 10A is characterized by any one of embodiments a-i wherein the XRPD pattern includes a peak at 20.9+/−0.4°2theta.
k. In certain embodiments S-6-MBPB Pattern 10A is characterized by any one of embodiments a-i wherein the XRPD pattern includes a peak at 21.6+/−0.4°2theta.
l. In certain embodiments S-6-MBPB Pattern 10A is characterized by any one of embodiments a-i wherein the XRPD pattern includes a peak at 21.9+/−0.4° 2theta.
m. In certain embodiments S-6-MBPB Pattern 10A is characterized by any one of embodiments a-i wherein the XRPD pattern includes a peak at 23.4+/−0.4° 2theta.
n. In certain embodiments S-6-MBPB Pattern 10A is characterized by any one of embodiments a-i wherein the XRPD pattern includes a peak at 25.3+/−0.4°2theta.
o. In certain embodiments S-6-MBPB Pattern 10A is characterized by any one of embodiments a-i wherein the XRPD pattern includes a peak at 27.2+/−0.4° 2theta.
p. In certain embodiments S-6-MBPB Pattern 10A is characterized by any one of embodiments a-o wherein the peaks are within +/−0.3°2theta.
q. In certain embodiments S-6-MBPB Pattern 10A is characterized by any one of embodiments a-o wherein the peaks are within +/−0.2°2theta.
r. In certain embodiments S-6-MBPB Pattern 10A is characterized by any one of embodiments a-q wherein at least three of the recited peaks have a relative peak intensity of at least 10%.
s. In certain embodiments S-6-MBPB Pattern 10A is characterized by any one of embodiments a-q wherein at least three of the recited peaks have a relative peak intensity of at least 20%.
t. In certain embodiments S-6-MBPB Pattern 10A is characterized by any one of embodiments a-q wherein at least four of the recited peaks have a relative peak intensity of at least 10%.
u. In certain embodiments S-6-MBPB Pattern 10A is characterized by any one of embodiments a-q wherein at least four of the recited peaks have a relative peak intensity of at least 20%.
v. In certain embodiments S-6-MBPB Pattern 10A is characterized by any one of embodiments a-q wherein at least five of the recited peaks have a relative peak intensity of at least 10%.
w. In certain embodiments S-6-MBPB Pattern 10A is characterized by any one of embodiments a-q wherein at least six of the recited peaks have a relative peak intensity of at least 20%.

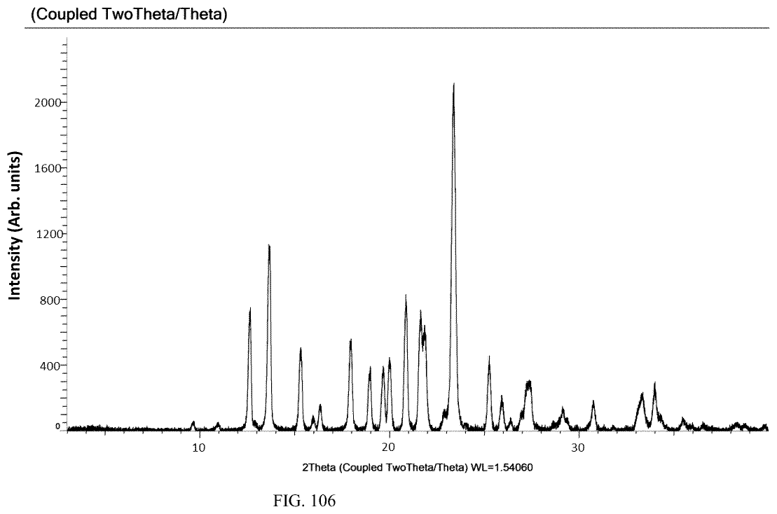

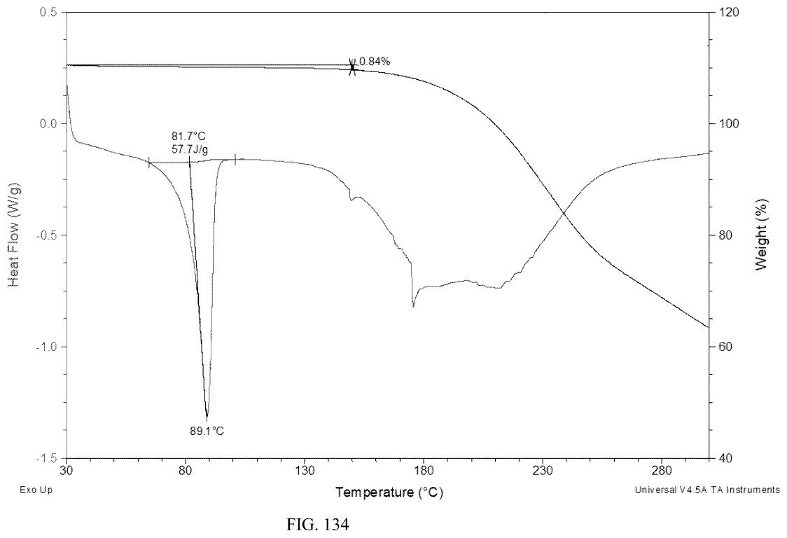

In certain embodiments S-6-MBPB Pattern 10A is characterized by the XRPD diffractogram in FIG. 106 and/or the DSC graph shown in FIG. 134.

### Citric Salt

In certain aspects the invention provides S-6-MBPB as citric salt. In certain embodiments the S-6-MBPB salt is a stable morphic form denoted Pattern 12A.
a. In certain embodiments S-6-MBPB Pattern 12A is characterized by an XRPD pattern with three or more peaks selected from 6.3, 12.4, 15.2, 17.5, 18.6, 19.6, 20.2, 24.8, and 27.7+/−0.4° 2theta.
b. In certain embodiments S-6-MBPB Pattern 12A is characterized by an XRPD pattern with four or more peaks selected from 6.3, 12.4, 15.2, 17.5, 18.6, 19.6, 20.2, 24.8, and 27.7+/−0.4 02theta.
c. In certain embodiments S-6-MBPB Pattern 12A is characterized by an XRPD pattern with five or more peaks selected from 6.3, 12.4, 15.2, 17.5, 18.6, 19.6, 20.2, 24.8, and 27.7+/−0.4°2theta.
d. In certain embodiments S-6-MBPB Pattern 12A is characterized by an XRPD pattern with six or more peaks selected from 6.3, 12.4, 15.2, 17.5, 18.6, 19.6, 20.2, 24.8, and 27.7+/−0.4 02theta.
e. In certain embodiments S-6-MBPB Pattern 12A is characterized by an XRPD pattern with seven or more peaks selected from 6.3, 12.4, 15.2, 17.5, 18.6, 19.6, 20.2, 24.8, and 27.7+/−0.4° 2theta.
f. In certain embodiments S-6-MBPB Pattern 12A is characterized by any one of embodiments a-e wherein the XRPD pattern includes a peak at 6.3+/−0.4° 2theta.
g. In certain embodiments S-6-MBPB Pattern 12A is characterized by any one of embodiments a-f wherein the XRPD pattern includes a peak at 6.9+/−0.4°2theta.
h. In certain embodiments S-6-MBPB Pattern 12A is characterized by any one of embodiments a-g wherein the XRPD pattern includes a peak at 12.4+/−0.4°2theta.
i. In certain embodiments S-6-MBPB Pattern 12A is characterized by any one of embodiments a-h wherein the XRPD pattern includes a peak at 15.2+/−0.4°2theta.
j. In certain embodiments S-6-MBPB Pattern 12A is characterized by any one of embodiments a-i wherein the XRPD pattern includes a peak at 17.5+/−0.4° 2theta.
k. In certain embodiments S-6-MBPB Pattern 12A is characterized by any one of embodiments a-i wherein the XRPD pattern includes a peak at 18.6+/−0.4°2theta.
l. In certain embodiments S-6-MBPB Pattern 12A is characterized by any one of embodiments a-i wherein the XRPD pattern includes a peak at 19.6+/−0.4° 2theta.
m. In certain embodiments S-6-MBPB Pattern 12A is characterized by any one of embodiments a-i wherein the XRPD pattern includes a peak at 20.2+/−0.4°2theta.
n. In certain embodiments S-6-MBPB Pattern 12A is characterized by any one of embodiments a-i wherein the XRPD pattern includes a peak at 24.8+/−0.4° 2theta.
o. In certain embodiments S-6-MBPB Pattern 12A is characterized by any one of embodiments a-i wherein the XRPD pattern includes a peak at 27.7+/−0.4°2theta.
p. In certain embodiments S-6-MBPB Pattern 12A is characterized by any one of embodiments a-o wherein the peaks are within +/−0.3°2theta.
q. In certain embodiments S-6-MBPB Pattern 12A is characterized by any one of embodiments a-o wherein the peaks are within +/−0.2°2theta.
r. In certain embodiments S-6-MBPB Pattern 12A is characterized by any one of embodiments a-q wherein at least three of the recited peaks have a relative peak intensity of at least 10%.
s. In certain embodiments S-6-MBPB Pattern 12A is characterized by any one of embodiments a-q wherein at least three of the recited peaks have a relative peak intensity of at least 20%.
t. In certain embodiments S-6-MBPB Pattern 12A is characterized by any one of embodiments a-q wherein at least four of the recited peaks have a relative peak intensity of at least 10%.
u. In certain embodiments S-6-MBPB Pattern 12A is characterized by any one of embodiments a-q wherein at least four of the recited peaks have a relative peak intensity of at least 20%.
v. In certain embodiments S-6-MBPB Pattern 12A is characterized by any one of embodiments a-q wherein at least five of the recited peaks have a relative peak intensity of at least 10%.
w. In certain embodiments S-6-MBPB Pattern 12A is characterized by any one of embodiments a-q wherein at least six of the recited peaks have a relative peak intensity of at least 20%.

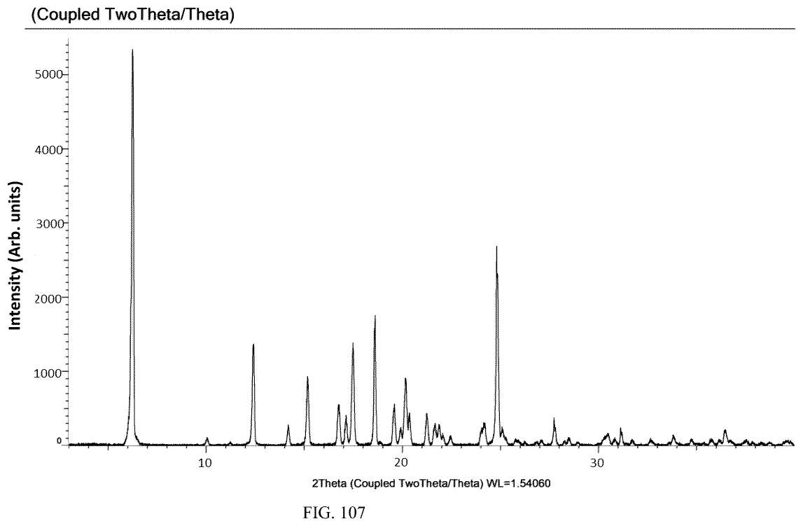

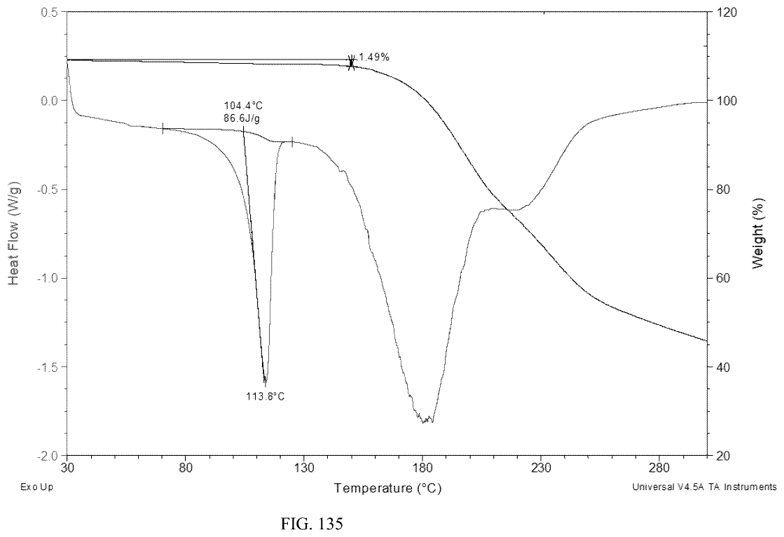

In certain embodiments S-6-MBPB Pattern 12A is characterized by the XRPD diffractogram in FIG. 107 and/or the DSC graph shown in FIG. 135.

### Pattern 13A

In certain aspects the invention provides S-6-MBPB as fumaric salt. In certain embodiments the S-6-MBPB salt is a stable morphic form denoted Pattern 13A.
a. In certain embodiments S-6-MBPB Pattern 13A is characterized by an XRPD pattern with three or more peaks selected from 12.3, 14.8, 17.2, 17.7, 19.2, 21.9, 22.3, 22.5, 24.2, 24.5, 25.2, 25.7, 26.1, 27.5, and 28.9+/−0.4° 2theta.
b. In certain embodiments S-6-MBPB Pattern 13A is characterized by an XRPD pattern with four or more peaks selected from 12.3, 14.8, 17.2, 17.7, 19.2, 21.9, 22.3, 22.5, 24.2, 24.5, 25.2, 25.7, 26.1, 27.5, and 28.9+/−0.4°2theta.
c. In certain embodiments S-6-MBPB Pattern 13A is characterized by an XRPD pattern with five or more peaks selected from 12.3, 14.8, 17.2, 17.7, 19.2, 21.9, 22.3, 22.5, 24.2, 24.5, 25.2, 25.7, 26.1, 27.5, and 28.9+/−0.4° 2theta.
d. In certain embodiments S-6-MBPB Pattern 13A is characterized by an XRPD pattern with six or more peaks selected from 12.3, 14.8, 17.2, 17.7, 19.2, 21.9, 22.3, 22.5, 24.2, 24.5, 25.2, 25.7, 26.1, 27.5, and 28.9+/−0.4° 2theta.
e. In certain embodiments S-6-MBPB Pattern 13A is characterized by an XRPD pattern with seven or more peaks selected from 12.3, 14.8, 17.2, 17.7, 19.2, 21.9, 22.3, 22.5, 24.2, 24.5, 25.2, 25.7, 26.1, 27.5, and 28.9+/−0.4° 2theta.
f. In certain embodiments S-6-MBPB Pattern 13A is characterized by any one of embodiments a-e wherein the XRPD pattern includes a peak at 12.3+/−0.4° 2theta.
g. In certain embodiments S-6-MBPB Pattern 13A is characterized by any one of embodiments a-f wherein the XRPD pattern includes a peak at 14.8+/−0.4°2theta.
h. In certain embodiments S-6-MBPB Pattern 13A is characterized by any one of embodiments a-g wherein the XRPD pattern includes a peak at 17.2+/−0.4°2theta.
i. In certain embodiments S-6-MBPB Pattern 13A is characterized by any one of embodiments a-h wherein the XRPD pattern includes a peak at 17.7+/−0.4°2theta.
j. In certain embodiments S-6-MBPB Pattern 13A is characterized by any one of embodiments a-i wherein the XRPD pattern includes a peak at 21.9+/−0.4° 2theta.
k. In certain embodiments S-6-MBPB Pattern 13A is characterized by any one of embodiments a-i wherein the XRPD pattern includes a peak at 22.3+/−0.4°2theta.
l. In certain embodiments S-6-MBPB Pattern 13A is characterized by any one of embodiments a-i wherein the XRPD pattern includes a peak at 24.5+/−0.4°2theta.
m. In certain embodiments S-6-MBPB Pattern 13A is characterized by any one of embodiments a-i wherein the XRPD pattern includes a peak at 25.7+/−0.4°2theta.
n. In certain embodiments S-6-MBPB Pattern 13A is characterized by any one of embodiments a-i wherein the XRPD pattern includes a peak at 26.1+/−0.4° 2theta.
o. In certain embodiments S-6-MBPB Pattern 13A is characterized by any one of embodiments a-i wherein the XRPD pattern includes a peak at 27.5+/−0.4°2theta.
p. In certain embodiments S-6-MBPB Pattern 13A is characterized by any one of embodiments a-o wherein the peaks are within +/−0.3°2theta.
q. In certain embodiments S-6-MBPB Pattern 13A is characterized by any one of embodiments a-o wherein the peaks are within +/−0.2°2theta.
r. In certain embodiments S-6-MBPB Pattern 13A is characterized by any one of embodiments a-q wherein at least three of the recited peaks have a relative peak intensity of at least 10%.
s. In certain embodiments S-6-MBPB Pattern 13A is characterized by any one of embodiments a-q wherein at least three of the recited peaks have a relative peak intensity of at least 20%.
t. In certain embodiments S-6-MBPB Pattern 13A is characterized by any one of embodiments a-q wherein at least four of the recited peaks have a relative peak intensity of at least 10%.
u. In certain embodiments S-6-MBPB Pattern 13A is characterized by any one of embodiments a-q wherein at least four of the recited peaks have a relative peak intensity of at least 20%.
v. In certain embodiments S-6-MBPB Pattern 13A is characterized by any one of embodiments a-q wherein at least five of the recited peaks have a relative peak intensity of at least 10%.
w. In certain embodiments S-6-MBPB Pattern 13A is characterized by any one of embodiments a-q wherein at least six of the recited peaks have a relative peak intensity of at least 20%.

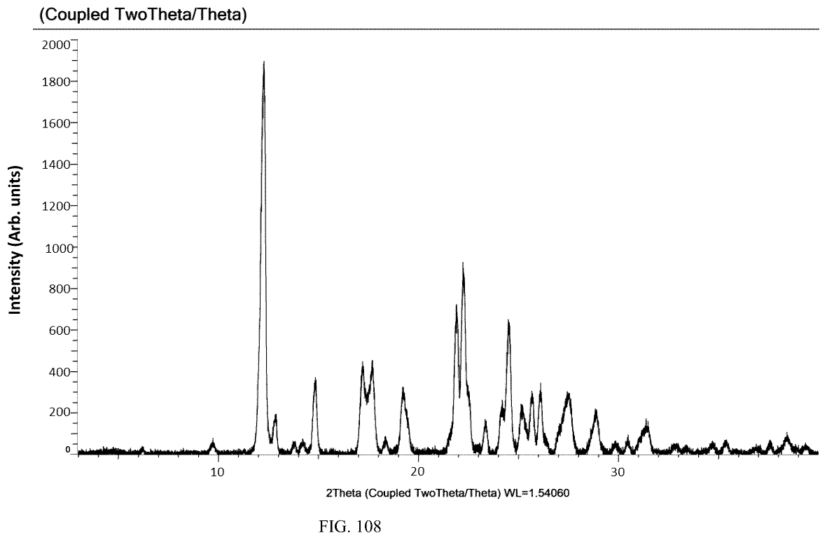

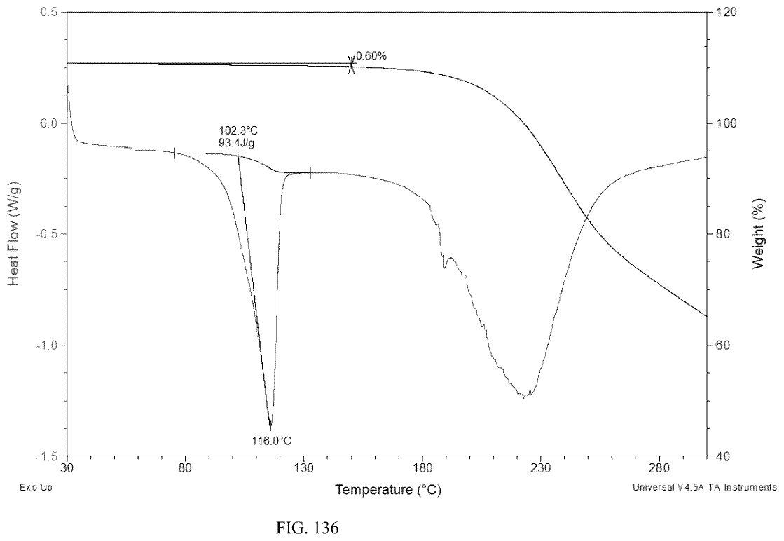

In certain embodiments S-6-MBPB Pattern 13A is characterized by the XRPD diffractogram in FIG. 108 and/or the DSC graph shown in FIG. 136.

### Pattern 13B

In certain aspects the invention provides S-6-MBPB as fumaric salt. In certain embodiments the S-6-MBPB salt is a stable morphic form denoted Pattern 13B.
a. In certain embodiments S-6-MBPB Pattern 13B is characterized by an XRPD pattern with three or more peaks selected from 7.5, 12.2, 13.4, 13.8, 14.4, 14.8, 15.3, 16.7, 17.1, 17.4, 17.7, 18.2, 18.8, 19.2, 20.5, 21.6, 21.9, 22.2, 23.2, 23.8, 24.2, 24.3, 24.6, 25.1, 25.3, 25.7, 26.9, 27.2, and 28.8+/−0.4°2theta.
b. In certain embodiments S-6-MBPB Pattern 13B is characterized by an XRPD pattern with four or more peaks selected from 7.5, 12.2, 13.4, 13.8, 14.4, 14.8, 15.3, 16.7, 17.1, 17.4, 17.7, 18.2, 18.8, 19.2, 20.5, 21.6, 21.9, 22.2, 23.2, 23.8, 24.2, 24.3, 24.6, 25.1, 25.3, 25.7, 26.9, 27.2, and 28.8+/−0.4° 2theta.
c. In certain embodiments S-6-MBPB Pattern 13B is characterized by an XRPD pattern with five or more peaks selected from 7.5, 12.2, 13.4, 13.8, 14.4, 14.8, 15.3, 16.7, 17.1, 17.4, 17.7, 18.2, 18.8, 19.2, 20.5, 21.6, 21.9, 22.2, 23.2, 23.8, 24.2, 24.3, 24.6, 25.1, 25.3, 25.7, 26.9, 27.2, and 28.8+/−0.4° 2theta.
d. In certain embodiments S-6-MBPB Pattern 13B is characterized by an XRPD pattern with six or more peaks selected from 7.5, 12.2, 13.4, 13.8, 14.4, 14.8, 15.3, 16.7, 17.1, 17.4, 17.7, 18.2, 18.8, 19.2, 20.5, 21.6, 21.9, 22.2, 23.2, 23.8, 24.2, 24.3, 24.6, 25.1, 25.3, 25.7, 26.9, 27.2, and 28.8+/−0.4° 2theta.
e. In certain embodiments S-6-MBPB Pattern 13B is characterized by an XRPD pattern with seven or more peaks selected from 7.5, 12.2, 13.4, 13.8, 14.4, 14.8, 15.3, 16.7, 17.1, 17.4, 17.7, 18.2, 18.8, 19.2, 20.5, 21.6, 21.9, 22.2, 23.2, 23.8, 24.2, 24.3, 24.6, 25.1, 25.3, 25.7, 26.9, 27.2, and 28.8+/−0.4° 2theta.
f. In certain embodiments S-6-MBPB Pattern 13B is characterized by any one of embodiments a-e wherein the XRPD pattern includes a peak at 12.2+/−0.4° 2theta.
g. In certain embodiments S-6-MBPB Pattern 13B is characterized by any one of embodiments a-f wherein the XRPD pattern includes a peak at 14.4+/−0.4° 2theta.
h. In certain embodiments S-6-MBPB Pattern 13B is characterized by any one of embodiments a-g wherein the XRPD pattern includes a peak at 15.3+/−0.4° 2theta.
i. In certain embodiments S-6-MBPB Pattern 13B is characterized by any one of embodiments a-h wherein the XRPD pattern includes a peak at 17.7+/−0.4°2theta.
j. In certain embodiments S-6-MBPB Pattern 13B is characterized by any one of embodiments a-i wherein the XRPD pattern includes a peak at 18.8+/−0.4°2theta.
k. In certain embodiments S-6-MBPB Pattern 13B is characterized by any one of embodiments a-i wherein the XRPD pattern includes a peak at 19.2+/−0.4° 2theta.
l. In certain embodiments S-6-MBPB Pattern 13B is characterized by any one of embodiments a-i wherein the XRPD pattern includes a peak at 20.5+/−0.4°2theta.
m. In certain embodiments S-6-MBPB Pattern 13B is characterized by any one of embodiments a-i wherein the XRPD pattern includes a peak at 21.9+/−0.4°2theta.
n. In certain embodiments S-6-MBPB Pattern 13B is characterized by any one of embodiments a-i wherein the XRPD pattern includes a peak at 22.2+/−0.4°2theta.
o. In certain embodiments S-6-MBPB Pattern 13B is characterized by any one of embodiments a-i wherein the XRPD pattern includes a peak at 24.2+/−0.4°2theta.
p. In certain embodiments S-6-MBPB Pattern 13B is characterized by any one of embodiments a-i wherein the XRPD pattern includes a peak at 25.3+/−0.4°2theta.
q. In certain embodiments S-6-MBPB Pattern 13B is characterized by any one of embodiments a-i wherein the XRPD pattern includes a peak at 28.8+/−0.4°2theta.
r. In certain embodiments S-6-MBPB Pattern 13B is characterized by any one of embodiments a-q wherein the peaks are within +/−0.3°2theta.
s. In certain embodiments S-6-MBPB Pattern 13B is characterized by any one of embodiments a-q wherein the peaks are within +/−0.2°2theta.
t. In certain embodiments S-6-MBPB Pattern 13B is characterized by any one of embodiments a-s wherein at least three of the recited peaks have a relative peak intensity of at least 10%.
u. In certain embodiments S-6-MBPB Pattern 13B is characterized by any one of embodiments a-s wherein at least three of the recited peaks have a relative peak intensity of at least 20%.
v. In certain embodiments S-6-MBPB Pattern 13B is characterized by any one of embodiments a-s wherein at least four of the recited peaks have a relative peak intensity of at least 10%.
w. In certain embodiments S-6-MBPB Pattern 13B is characterized by any one of embodiments a-s wherein at least four of the recited peaks have a relative peak intensity of at least 20%.

x. In certain embodiments S-6-MBPB Pattern 13B is characterized by any one of embodiments a-q wherein at least five of the recited peaks have a relative peak intensity of at least 10%.

y. In certain embodiments S-6-MBPB Pattern 13B is characterized by any one of embodiments a-s wherein at least six of the recited peaks have a relative peak intensity of at least 20%.

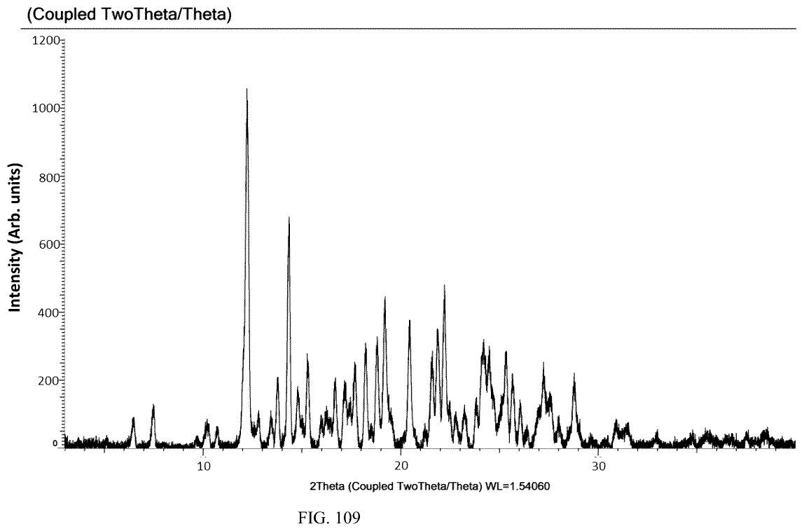

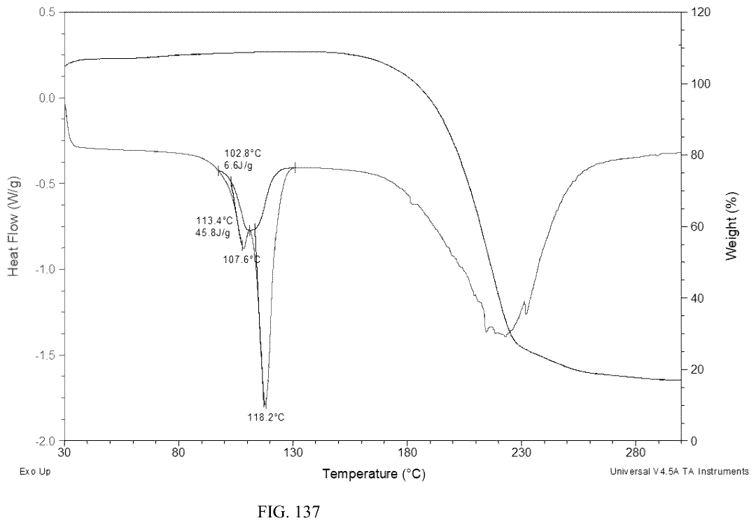

In certain embodiments S-6-MBPB Pattern 13B is characterized by the XRPD diffractogram in FIG. 109 and/or the DSC graph shown in FIG. 137.

### Morphic Forms of S-5-MBPB

### HCl Salt

In certain aspects the invention provides S-5-MBPB as HCl salt. In certain embodiments the S-5-MBPB salt is a stable morphic form denoted Pattern 1A.
a. In certain embodiments S-5-MBPB Pattern 1A is characterized by an XRPD pattern with three or more peaks selected from 5.9, 8.1, 12.7, 13.2, 13.7, 14.5, 16.4, 17.5, 17.9, 18.2, 18.8, 19.2, 19.9, 20.7, 20.9, 21.9, 22.2, 244, 25.4, 25.5, 25.7, 27.5, 27.7, 30.3, and 32.2+/−0.4°2theta.
b. In certain embodiments S-5-MBPB Pattern 1A is characterized by an XRPD pattern with four or more peaks selected from 5.9, 8.1, 12.7, 13.2, 13.7, 14.5, 16.4, 17.5, 17.9, 18.2, 18.8, 19.2, 19.9, 20.7, 20.9, 21.9, 22.2, 244, 25.4, 25.5, 25.7, 27.5, 27.7, 30.3, and 32.2+/−0.4°2theta.
c. In certain embodiments S-5-MBPB Pattern 1A is characterized by an XRPD pattern with five or more peaks selected from 5.9, 8.1, 12.7, 13.2, 13.7, 14.5, 16.4, 17.5, 17.9, 18.2, 18.8, 19.2, 19.9, 20.7, 20.9, 21.9, 22.2, 244, 25.4, 25.5, 25.7, 27.5, 27.7, 30.3, and 32.2+/−0.4°2theta.
d. In certain embodiments S-5-MBPB Pattern 1A is characterized by an XRPD pattern with six or more peaks selected from 5.9, 8.1, 12.7, 13.2, 13.7, 14.5, 16.4, 17.5, 17.9, 18.2, 18.8, 19.2, 19.9, 20.7, 20.9, 21.9, 22.2, 244, 25.4, 25.5, 25.7, 27.5, 27.7, 30.3, and 32.2+/−0.4° 2theta.
e. In certain embodiments S-5-MBPB Pattern 1A is characterized by an XRPD pattern with seven or more peaks selected from 5.9, 8.1, 12.7, 13.2, 13.7, 14.5, 16.4, 17.5, 17.9, 18.2, 18.8, 19.2, 19.9, 20.7, 20.9, 21.9, 22.2, 244, 25.4, 25.5, 25.7, 27.5, 27.7, 30.3, and 32.2+/−0.4°2theta.
f. In certain embodiments S-5-MBPB Pattern 1A is characterized by any one of embodiments a-e wherein the XRPD pattern includes a peak at 5.9+/−0.4° 2theta.
g. In certain embodiments S-5-MBPB Pattern 1A is characterized by any one of embodiments a-f wherein the XRPD pattern includes a peak at 13.7+/−0.4°2theta.
h. In certain embodiments S-5-MBPB Pattern 1A is characterized by any one of embodiments a-g wherein the XRPD pattern includes a peak at 14.5+/−0.4°2theta.
i. In certain embodiments S-5-MBPB Pattern 1A is characterized by any one of embodiments a-h wherein the XRPD pattern includes a peak at 16.4+/−0.4°2theta.
j. In certain embodiments S-5-MBPB Pattern 1A is characterized by any one of embodiments a-i wherein the XRPD pattern includes a peak at 17.5+/−0.4°2theta.
k. In certain embodiments S-5-MBPB Pattern 1A is characterized by any one of embodiments a-i wherein the XRPD pattern includes a peak at 18.2+/−0.4°2theta.
l. In certain embodiments S-5-MBPB Pattern 1A is characterized by any one of embodiments a-i wherein the XRPD pattern includes a peak at 18.8+/−0.4°2theta.
m. In certain embodiments S-5-MBPB Pattern 1A is characterized by any one of embodiments a-i wherein the XRPD pattern includes a peak at 21.9+/−0.4°2theta.
n. In certain embodiments S-5-MBPB Pattern 1A is characterized by any one of embodiments a-i wherein the XRPD pattern includes a peak at 25.4+/−0.4°2theta.
o. In certain embodiments S-5-MBPB Pattern 1A is characterized by any one of embodiments a-i wherein the XRPD pattern includes a peak at 27.7+/−0.4°2theta.
p. In certain embodiments S-5-MBPB Pattern 1A is characterized by any one of embodiments a-o wherein the peaks are within +/−0.3°2theta.
q. In certain embodiments S-5-MBPB Pattern 1A is characterized by any one of embodiments a-o wherein the peaks are within +/−0.2°2theta.
r. In certain embodiments S-5-MBPB Pattern 1A is characterized by any one of embodiments a-q wherein at least three of the recited peaks have a relative peak intensity of at least 10%.
s. In certain embodiments S-5-MBPB Pattern 1A is characterized by any one of embodiments a-q wherein at least three of the recited peaks have a relative peak intensity of at least 20%.
t. In certain embodiments S-5-MBPB Pattern 1A is characterized by any one of embodiments a-q wherein at least four of the recited peaks have a relative peak intensity of at least 10%.
u. In certain embodiments S-5-MBPB Pattern 1A is characterized by any one of embodiments a-q wherein at least four of the recited peaks have a relative peak intensity of at least 20%.
v. In certain embodiments S-5-MBPB Pattern 1A is characterized by any one of embodiments a-q wherein at least five of the recited peaks have a relative peak intensity of at least 10%.
w. In certain embodiments S-5-MBPB Pattern 1A is characterized by any one of embodiments a-q wherein at least six of the recited peaks have a relative peak intensity of at least 20%.

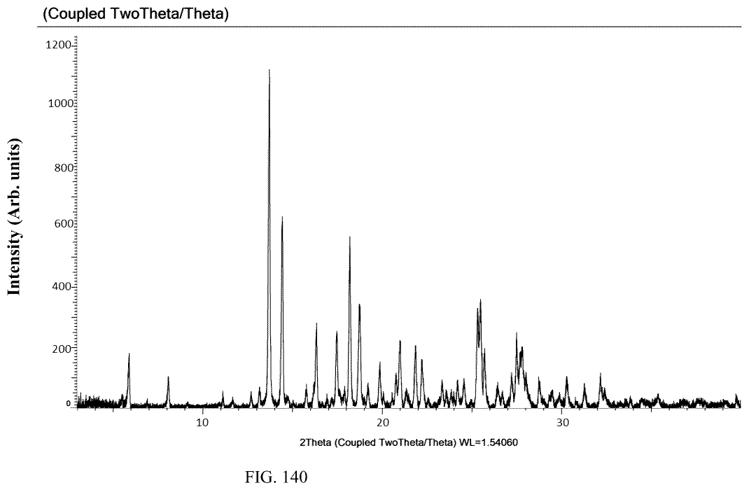

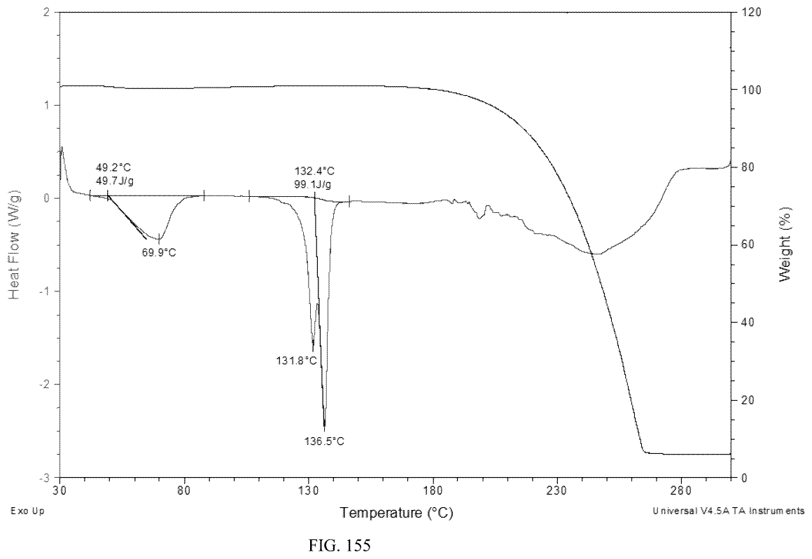

In certain embodiments S-5-MBPB Pattern 1A is characterized by the XRPD diffractogram in FIG. 140 and/or the DSC graph shown in FIG. 155.

### HBr Salt

In certain aspects the invention provides S-5-MBPB as HBr salt. In certain embodiments the S-5-MBPB salt is a stable morphic form denoted Pattern 2B.
a. In certain embodiments S-5-MBPB Pattern 2B is characterized by an XRPD pattern with three or more peaks selected from 18.4, 18.6, 19.8, 19.9, 20.9, 23.7, 24.6, 24.7, 26.4, 26.6, 26.7, 29.0, 31.7, and 32.8+/−0.4° 2theta.
b. In certain embodiments S-5-MBPB Pattern 2B is characterized by an XRPD pattern with four or more peaks selected from 18.4, 18.6, 19.8, 19.9, 20.9, 23.7, 24.6, 24.7, 26.4, 26.6, 26.7, 29.0, 31.7, and 32.8+/−0.4° 2theta.
c. In certain embodiments S-5-MBPB Pattern 2B is characterized by an XRPD pattern with five or more peaks selected from 18.4, 18.6, 19.8, 19.9, 20.9, 23.7, 24.6, 24.7, 26.4, 26.6, 26.7, 29.0, 31.7, and 32.8+/−0.4°2theta.
d. In certain embodiments S-5-MBPB Pattern 2B is characterized by an XRPD pattern with six or more peaks selected from 18.4, 18.6, 19.8, 19.9, 20.9, 23.7, 24.6, 24.7, 26.4, 26.6, 26.7, 29.0, 31.7, and 32.8+/−0.4°2theta.
e. In certain embodiments S-5-MBPB Pattern 2B is characterized by an XRPD pattern with seven or more peaks selected from 18.4, 18.6, 19.8, 19.9, 20.9, 23.7, 24.6, 24.7, 26.4, 26.6, 26.7, 29.0, 31.7, and 32.8+/−0.4° 2theta.
f. In certain embodiments S-5-MBPB Pattern 2B is characterized by any one of embodiments a-e wherein the XRPD pattern includes a peak at 18.4+/−0.4° 2theta.
g. In certain embodiments S-5-MBPB Pattern 2B is characterized by any one of embodiments a-f wherein the XRPD pattern includes a peak at 18.6+/−0.4° 2theta.
h. In certain embodiments S-5-MBPB Pattern 2B is characterized by any one of embodiments a-g wherein the XRPD pattern includes a peak at 19.8+/−0.4°2theta.
i. In certain embodiments S-5-MBPB Pattern 2B is characterized by any one of embodiments a-h wherein the XRPD pattern includes a peak at 23.7+/−0.4° 2theta.
j. In certain embodiments S-5-MBPB Pattern 2B is characterized by any one of embodiments a-i wherein the XRPD pattern includes a peak at 26.4+/−0.4° 2theta.
k. In certain embodiments S-5-MBPB Pattern 2B is characterized by any one of embodiments a-i wherein the XRPD pattern includes a peak at 26.7+/−0.4°2theta.
l. In certain embodiments S-5-MBPB Pattern 2B is characterized by any one of embodiments a-i wherein the XRPD pattern includes a peak at 29.0+/−0.4°2theta.
m. In certain embodiments S-5-MBPB Pattern 2B is characterized by any one of embodiments a-i wherein the XRPD pattern includes a peak at 31.7+/−0.4°2theta.
n. In certain embodiments S-5-MBPB Pattern 2B is characterized by any one of embodiments a-i wherein the XRPD pattern includes a peak at 32.8+/−0.4°2theta.
o. In certain embodiments S-5-MBPB Pattern 2B is characterized by any one of embodiments a-i wherein the XRPD pattern includes a peak at 33.9+/−0.4°2theta.
p. In certain embodiments S-5-MBPB Pattern 2B is characterized by any one of embodiments a-o wherein the peaks are within +/−0.3°2theta.
q. In certain embodiments S-5-MBPB Pattern 2B is characterized by any one of embodiments a-o wherein the peaks are within +/−0.2°2theta.
r. In certain embodiments S-5-MBPB Pattern 2B is characterized by any one of embodiments a-q wherein at least three of the recited peaks have a relative peak intensity of at least 10%.
s. In certain embodiments S-5-MBPB Pattern 2B is characterized by any one of embodiments a-q wherein at least three of the recited peaks have a relative peak intensity of at least 20%.
t. In certain embodiments S-5-MBPB Pattern 2B is characterized by any one of embodiments a-q wherein at least four of the recited peaks have a relative peak intensity of at least 10%.
u. In certain embodiments S-5-MBPB Pattern 2B is characterized by any one of embodiments a-q wherein at least four of the recited peaks have a relative peak intensity of at least 20%.
v. In certain embodiments S-5-MBPB Pattern 2B is characterized by any one of embodiments a-q wherein at least five of the recited peaks have a relative peak intensity of at least 10%.
w. In certain embodiments S-5-MBPB Pattern 2B is characterized by any one of embodiments a-q wherein at least six of the recited peaks have a relative peak intensity of at least 20%.

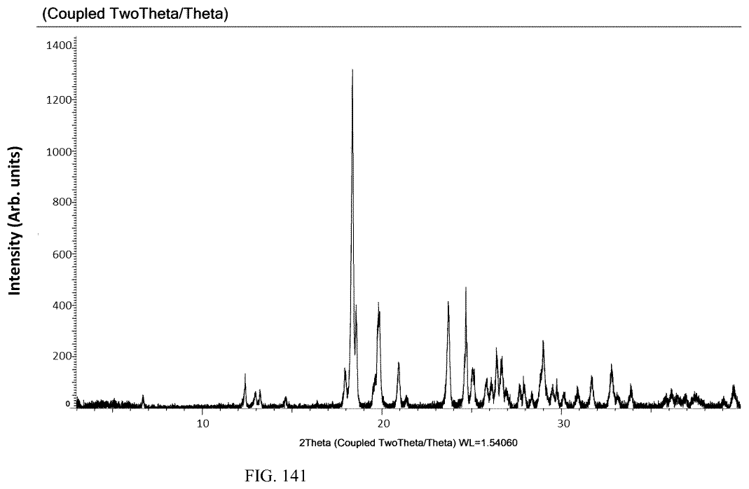

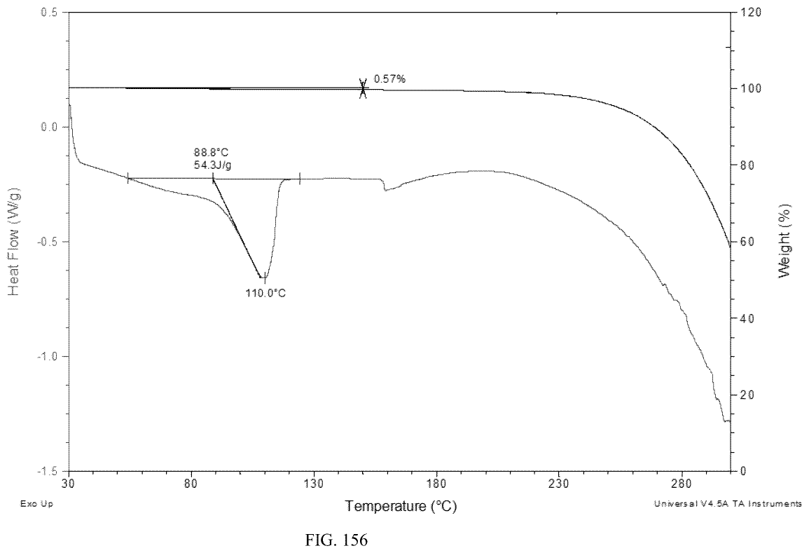

In certain embodiments S-5-MBPB Pattern 2B is characterized by the XRPD diffractogram in FIG. 141 and/or the DSC graph shown in FIG. 156.

H₃PO₄ Salt

In certain aspects the invention provides S-5-MBPB as H₃PO₄ salt. In certain embodiments the S-5-MBPB salt is a stable morphic form denoted Pattern 3A.
a. In certain embodiments S-5-MBPB Pattern 3A is characterized by an XRPD pattern with three or more peaks selected from 6.5, 12.8, 17.1, 19.2, 19.6, 21.0, 21.4, 21.9, and 26.6+/−0.4 02theta.
b. In certain embodiments S-5-MBPB Pattern 3A is characterized by an XRPD pattern with four or more peaks selected from 6.5, 12.8, 17.1, 19.2, 19.6, 21.0, 21.4, 21.9, and 26.6+/−0.4°2theta.
c. In certain embodiments S-5-MBPB Pattern 3A is characterized by an XRPD pattern with five or more peaks selected from 6.5, 12.8, 17.1, 19.2, 19.6, 21.0, 21.4, 21.9, and 26.6+/−0.4 02theta.
d. In certain embodiments S-5-MBPB Pattern 3A is characterized by an XRPD pattern with six or more peaks selected from 6.5, 12.8, 17.1, 19.2, 19.6, 21.0, 21.4, 21.9, and 26.6+/−0.4 02theta.
e. In certain embodiments S-5-MBPB Pattern 3A is characterized by an XRPD pattern with seven or more peaks selected from 6.5, 12.8, 17.1, 19.2, 19.6, 21.0, 21.4, 21.9, and 26.6+/−0.4° 2theta.
f. In certain embodiments S-5-MBPB Pattern 3A is characterized by any one of embodiments a-e wherein the XRPD pattern includes a peak at 6.5+/−0.4° 2theta.
g. In certain embodiments S-5-MBPB Pattern 3A is characterized by any one of embodiments a-f wherein the XRPD pattern includes a peak at 12.8+/−0.4° 2theta.
h. In certain embodiments S-5-MBPB Pattern 3A is characterized by any one of embodiments a-g wherein the XRPD pattern includes a peak at 17.1+/−0.4°2theta.
i. In certain embodiments S-5-MBPB Pattern 3A is characterized by any one of embodiments a-h wherein the XRPD pattern includes a peak at 18.6+/−0.4°2theta.
j. In certain embodiments S-5-MBPB Pattern 3A is characterized by any one of embodiments a-i wherein the XRPD pattern includes a peak at 19.2+/−0.4° 2theta.
k. In certain embodiments S-5-MBPB Pattern 3A is characterized by any one of embodiments a-i wherein the XRPD pattern includes a peak at 19.6+/−0.4°2theta.
l. In certain embodiments S-5-MBPB Pattern 3A is characterized by any one of embodiments a-i wherein the XRPD pattern includes a peak at 21.0+/−0.4°2theta.
m. In certain embodiments S-5-MBPB Pattern 3A is characterized by any one of embodiments a-i wherein the XRPD pattern includes a peak at 21.4+/−0.4°2theta.
n. In certain embodiments S-5-MBPB Pattern 3A is characterized by any one of embodiments a-i wherein the XRPD pattern includes a peak at 21.9+/−0.4°2theta.
o. In certain embodiments S-5-MBPB Pattern 3A is characterized by any one of embodiments a-i wherein the XRPD pattern includes a peak at 26.6+/−0.4° 2theta.
p. In certain embodiments S-5-MBPB Pattern 3A is characterized by any one of embodiments a-o wherein the peaks are within +/−0.3°2theta.
q. In certain embodiments S-5-MBPB Pattern 3A is characterized by any one of embodiments a-o wherein the peaks are within +/−0.2°2theta.
r. In certain embodiments S-5-MBPB Pattern 3A is characterized by any one of embodiments a-q wherein at least three of the recited peaks have a relative peak intensity of at least 10%.
s. In certain embodiments S-5-MBPB Pattern 3A is characterized by any one of embodiments a-q wherein at least three of the recited peaks have a relative peak intensity of at least 20%.
t. In certain embodiments S-5-MBPB Pattern 3A is characterized by any one of embodiments a-q wherein at least four of the recited peaks have a relative peak intensity of at least 10%.
u. In certain embodiments S-5-MBPB Pattern 3A is characterized by any one of embodiments a-q wherein at least four of the recited peaks have a relative peak intensity of at least 20%.
v. In certain embodiments S-5-MBPB Pattern 3A is characterized by any one of embodiments a-q wherein at least five of the recited peaks have a relative peak intensity of at least 10%.
w. In certain embodiments S-5-MBPB Pattern 3A is characterized by any one of embodiments a-q wherein at least six of the recited peaks have a relative peak intensity of at least 20%.

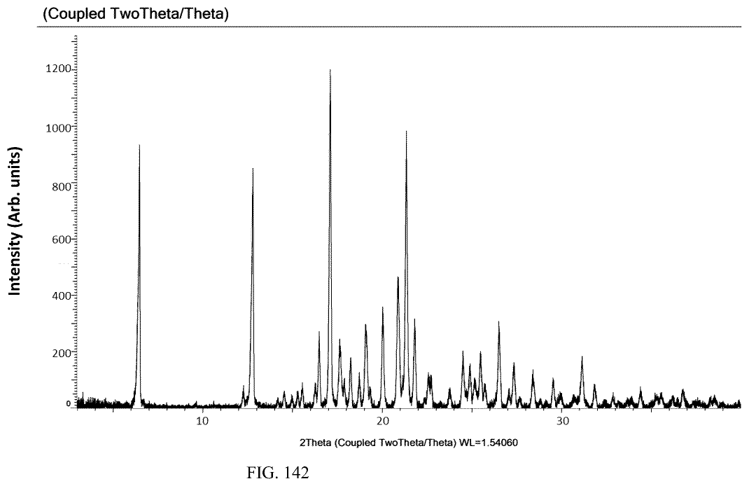

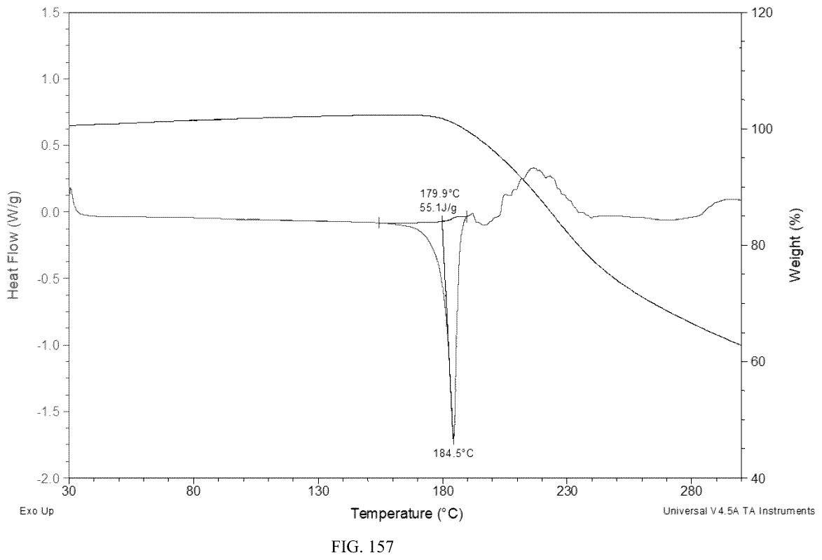

In certain embodiments S-5-MBPB Pattern 3A is characterized by the XRPD diffractogram in FIG. 142 and/or the DSC graph shown in FIG. 157.

### Succinic Salt

In certain aspects the invention provides S-5-MBPB as succinic salt. In certain embodiments the S-5-MBPB salt is a stable morphic form denoted Pattern 6A.
a. In certain embodiments S-5-MBPB Pattern 6A is characterized by an XRPD pattern with three or more peaks selected from 14.8, 15.6, 18.0, 18.6, 18.8, 21.3, 23.8, 24.4, 25.5, and 26.0+/−0.4° 2theta.
b. In certain embodiments S-5-MBPB Pattern 6A is characterized by an XRPD pattern with four or more peaks selected from 14.8, 15.6, 18.0, 18.6, 18.8, 21.3, 23.8, 24.4, 25.5, and 26.0+/−0.4°2theta.
c. In certain embodiments S-5-MBPB Pattern 6A is characterized by an XRPD pattern with five or more peaks selected from 14.8, 15.6, 18.0, 18.6, 18.8, 21.3, 23.8, 24.4, 25.5, and 26.0+/−0.4° 2theta.
d. In certain embodiments S-5-MBPB Pattern 6A is characterized by an XRPD pattern with six or more peaks selected from 14.8, 15.6, 18.0, 18.6, 18.8, 21.3, 23.8, 24.4, 25.5, and 26.0+/−0.4° 2theta.
e. In certain embodiments S-5-MBPB Pattern 6A is characterized by an XRPD pattern with seven or more peaks selected from 14.8, 15.6, 18.0, 18.6, 18.8, 21.3, 23.8, 24.4, 25.5, and 26.0+/−0.4° 2theta.
f. In certain embodiments S-5-MBPB Pattern 6A is characterized by any one of embodiments a-e wherein the XRPD pattern includes a peak at 14.8+/−0.4° 2theta.
g. In certain embodiments S-5-MBPB Pattern 6A is characterized by any one of embodiments a-f wherein the XRPD pattern includes a peak at 15.6+/−0.4° 2theta.
h. In certain embodiments S-5-MBPB Pattern 6A is characterized by any one of embodiments a-g wherein the XRPD pattern includes a peak at 18.0+/−0.4°2theta.
i. In certain embodiments S-5-MBPB Pattern 6A is characterized by any one of embodiments a-h wherein the XRPD pattern includes a peak at 18.6+/−0.4° 2theta.
j. In certain embodiments S-5-MBPB Pattern 6A is characterized by any one of embodiments a-i wherein the XRPD pattern includes a peak at 18.8+/−0.4°2theta.
k. In certain embodiments S-5-MBPB Pattern 6A is characterized by any one of embodiments a-i wherein the XRPD pattern includes a peak at 21.3+/−0.4°2theta.
l. In certain embodiments S-5-MBPB Pattern 6A is characterized by any one of embodiments a-i wherein the XRPD pattern includes a peak at 23.8+/−0.4° 2theta.
m. In certain embodiments S-5-MBPB Pattern 6A is characterized by any one of embodiments a-i wherein the XRPD pattern includes a peak at 24.4+/−0.4° 2theta.
n. In certain embodiments S-5-MBPB Pattern 6A is characterized by any one of embodiments a-i wherein the XRPD pattern includes a peak at 25.5+/−0.4°2theta.
o. In certain embodiments S-5-MBPB Pattern 6A is characterized by any one of embodiments a-i wherein the XRPD pattern includes a peak at 26.0+/−0.4°2theta.
p. In certain embodiments S-5-MBPB Pattern 6A is characterized by any one of embodiments a-o wherein the peaks are within +/−0.3°2theta.
q. In certain embodiments S-5-MBPB Pattern 6A is characterized by any one of embodiments a-o wherein the peaks are within +/−0.2° 2theta.
r. In certain embodiments S-5-MBPB Pattern 6A is characterized by any one of embodiments a-q wherein at least three of the recited peaks have a relative peak intensity of at least 10%.
s. In certain embodiments S-5-MBPB Pattern 6A is characterized by any one of embodiments a-q wherein at least three of the recited peaks have a relative peak intensity of at least 20%.
t. In certain embodiments S-5-MBPB Pattern 6A is characterized by any one of embodiments a-q wherein at least four of the recited peaks have a relative peak intensity of at least 10%.
u. In certain embodiments S-5-MBPB Pattern 6A is characterized by any one of embodiments a-q wherein at least four of the recited peaks have a relative peak intensity of at least 20%.
v. In certain embodiments S-5-MBPB Pattern 6A is characterized by any one of embodiments a-q wherein at least five of the recited peaks have a relative peak intensity of at least 10%.
w. In certain embodiments S-5-MBPB Pattern 6A is characterized by any one of embodiments a-q wherein at least six of the recited peaks have a relative peak intensity of at least 20%.

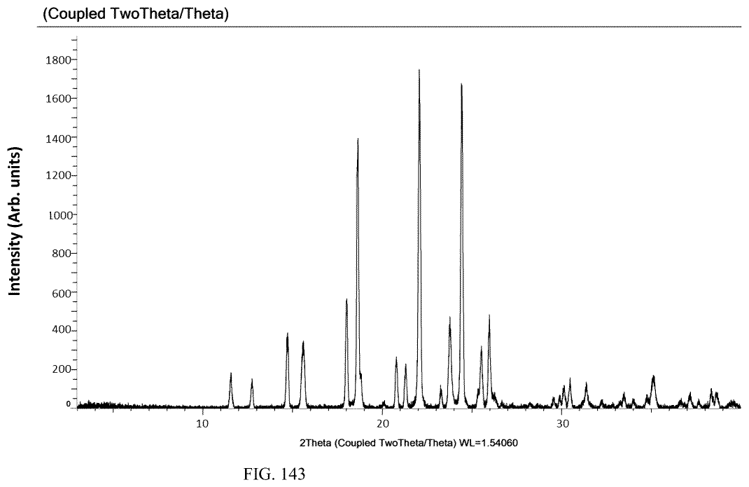

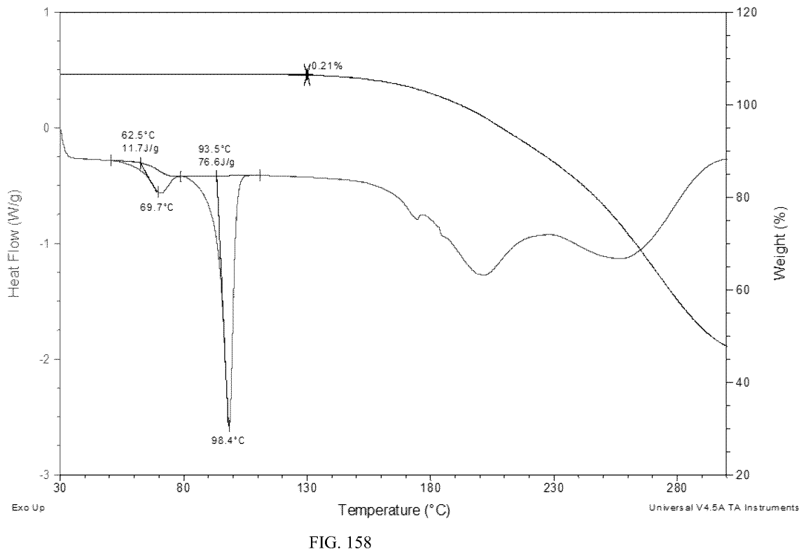

In certain embodiments S-5-MBPB Pattern 6A is characterized by the XRPD diffractogram in FIG. 143 and/or the DSC graph shown in FIG. 158.

### Maleic Salt

In certain aspects the invention provides S-5-MBPB as maleic salt. In certain embodiments the S-5-MBPB salt is a stable morphic form denoted Pattern 8A.
a. In certain embodiments S-5-MBPB Pattern 8A is characterized by an XRPD pattern with three or more peaks selected from 5.7, 9.9, 12.7, 14.8, 17.0, 19.6, 22.0, 22.7, 24.6, 27.4, and 28.5+/−0.4° 2theta.
b. In certain embodiments S-5-MBPB Pattern 8A is characterized by an XRPD pattern with four or more peaks selected from 5.7, 9.9, 12.7, 14.8, 17.0, 19.6, 22.0, 22.7, 24.6, 27.4, and 28.5+/−0.4° 2theta.
c. In certain embodiments S-5-MBPB Pattern 8A is characterized by an XRPD pattern with five or more peaks selected from 5.7, 9.9, 12.7, 14.8, 17.0, 19.6, 22.0, 22.7, 24.6, 27.4, and 28.5+/−0.4° 2theta.
d. In certain embodiments S-5-MBPB Pattern 8A is characterized by an XRPD pattern with six or more peaks selected from 5.7, 9.9, 12.7, 14.8, 17.0, 19.6, 22.0, 22.7, 24.6, 27.4, and 28.5+/−0.4° 2theta.
e. In certain embodiments S-5-MBPB Pattern 8A is characterized by an XRPD pattern with seven or more peaks selected from 5.7, 9.9, 12.7, 14.8, 17.0, 19.6, 22.0, 22.7, 24.6, 27.4, and 28.5+/−0.4° 2theta.
f. In certain embodiments S-5-MBPB Pattern 8A is characterized by any one of embodiments a-e wherein the XRPD pattern includes a peak at 5.7+/−0.4° 2theta.
g. In certain embodiments S-5-MBPB Pattern 8A is characterized by any one of embodiments a-f wherein the XRPD pattern includes a peak at 9.9+/−0.4°2theta.
h. In certain embodiments S-5-MBPB Pattern 8A is characterized by any one of embodiments a-g wherein the XRPD pattern includes a peak at 12.7+/−0.4°2theta.
i. In certain embodiments S-5-MBPB Pattern 8A is characterized by any one of embodiments a-h wherein the XRPD pattern includes a peak at 14.8+/−0.4° 2theta.
j. In certain embodiments S-5-MBPB Pattern 8A is characterized by any one of embodiments a-i wherein the XRPD pattern includes a peak at 17.0+/−0.4°2theta.
k. In certain embodiments S-5-MBPB Pattern 8A is characterized by any one of embodiments a-i wherein the XRPD pattern includes a peak at 19.6+/−0.4°2theta.
l. In certain embodiments S-5-MBPB Pattern 8A is characterized by any one of embodiments a-i wherein the XRPD pattern includes a peak at 22.0+/−0.4°2theta.
m. In certain embodiments S-5-MBPB Pattern 8A is characterized by any one of embodiments a-i wherein the XRPD pattern includes a peak at 22.7+/−0.4°2theta.
n. In certain embodiments S-5-MBPB Pattern 8A is characterized by any one of embodiments a-i wherein the XRPD pattern includes a peak at 24.6+/−0.4°2theta.
o. In certain embodiments S-5-MBPB Pattern 8A is characterized by any one of embodiments a-i wherein the XRPD pattern includes a peak at 28.5+/−0.4°2theta.
p. In certain embodiments S-5-MBPB Pattern 8A is characterized by any one of embodiments a-o wherein the peaks are within +/−0.3°2theta.
q. In certain embodiments S-5-MBPB Pattern 8A is characterized by any one of embodiments a-o wherein the peaks are within +/−0.2°2theta.
r. In certain embodiments S-5-MBPB Pattern 8A is characterized by any one of embodiments a-q wherein at least three of the recited peaks have a relative peak intensity of at least 10%.
s. In certain embodiments S-5-MBPB Pattern 8A is characterized by any one of embodiments a-q wherein at least three of the recited peaks have a relative peak intensity of at least 20%.
t. In certain embodiments S-5-MBPB Pattern 8A is characterized by any one of embodiments a-q wherein at least four of the recited peaks have a relative peak intensity of at least 10%.
u. In certain embodiments S-5-MBPB Pattern 8A is characterized by any one of embodiments a-q wherein at least four of the recited peaks have a relative peak intensity of at least 20%.
v. In certain embodiments S-5-MBPB Pattern 8A is characterized by any one of embodiments a-q wherein at least five of the recited peaks have a relative peak intensity of at least 10%.
w. In certain embodiments S-5-MBPB Pattern 8A is characterized by any one of embodiments a-q wherein at least six of the recited peaks have a relative peak intensity of at least 20%.

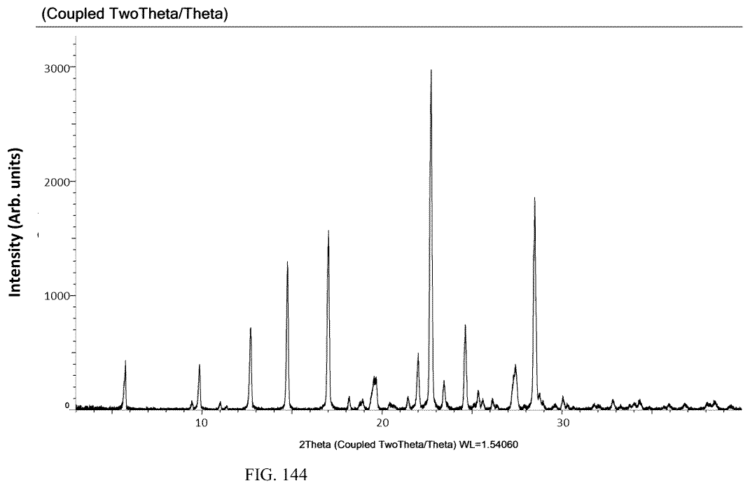

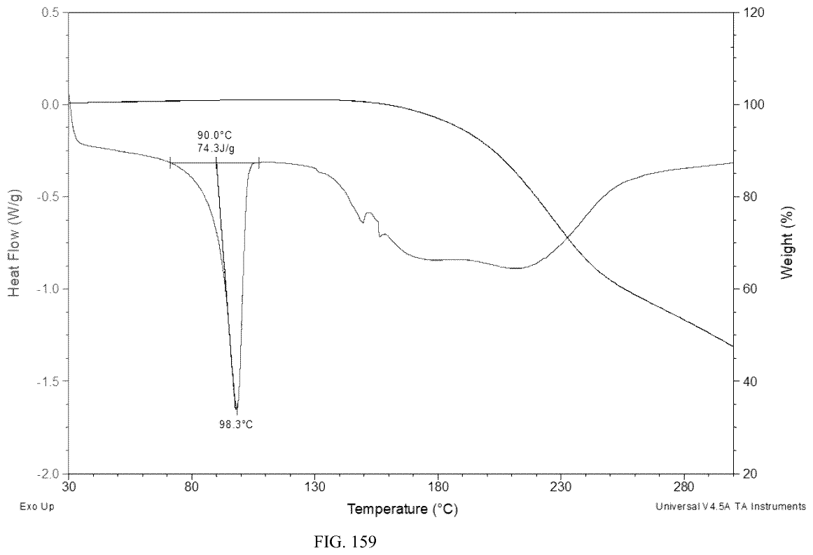

In certain embodiments S-5-MBPB Pattern 8A is characterized by the XRPD diffractogram in FIG. 144 and/or the DSC graph shown in FIG. 159.

### Succinic Salt

In certain aspects the invention provides S-5-MBPB as citric salt. In certain embodiments the S-5-MBPB salt is a stable morphic form denoted Pattern 9A.
a. In certain embodiments S-5-MBPB Pattern 9A is characterized by an XRPD pattern with three or more peaks selected from 6.5, 12.7, 16.4, 17.6, 19.0, 21.2, 21.7, and 25.3+/−0.4°2theta.
b. In certain embodiments S-5-MBPB Pattern 9A is characterized by an XRPD pattern with four or more peaks selected from 6.5, 12.7, 16.4, 17.6, 19.0, 21.2, 21.7, and 25.3+/−0.4°2theta.
c. In certain embodiments S-5-MBPB Pattern 9A is characterized by an XRPD pattern with five or more peaks selected from 6.5, 12.7, 16.4, 17.6, 19.0, 21.2, 21.7, and 25.3+/−0.4 02theta.
d. In certain embodiments S-5-MBPB Pattern 9A is characterized by an XRPD pattern with six or more peaks selected from 6.5, 12.7, 16.4, 17.6, 19.0, 21.2, 21.7, and 25.3+/−0.4°2theta.
e. In certain embodiments S-5-MBPB Pattern 9A is characterized by an XRPD pattern with seven or more peaks selected from 6.5, 12.7, 16.4, 17.6, 19.0, 21.2, 21.7, and 25.3+/−0.4 02theta.
f. In certain embodiments S-5-MBPB Pattern 9A is characterized by any one of embodiments a-e wherein the XRPD pattern includes a peak at 6.5+/−0.4° 2theta.
g. In certain embodiments S-5-MBPB Pattern 9A is characterized by any one of embodiments a-f wherein the XRPD pattern includes a peak at 12.7+/−0.4° 2theta.
h. In certain embodiments S-5-MBPB Pattern 9A is characterized by any one of embodiments a-g wherein the XRPD pattern includes a peak at 16.4+/−0.4° 2theta.
i. In certain embodiments S-5-MBPB Pattern 9A is characterized by any one of embodiments a-h wherein the XRPD pattern includes a peak at 17.6+/−0.4°2theta.
j. In certain embodiments S-5-MBPB Pattern 9A is characterized by any one of embodiments a-i wherein the XRPD pattern includes a peak at 18.5+/−0.4°2theta.
k. In certain embodiments S-5-MBPB Pattern 9A is characterized by any one of embodiments a-i wherein the XRPD pattern includes a peak at 19.0+/−0.4°2theta.
l. In certain embodiments S-5-MBPB Pattern 9A is characterized by any one of embodiments a-i wherein the XRPD pattern includes a peak at 19.5+/−0.4° 2theta.
m. In certain embodiments S-5-MBPB Pattern 9A is characterized by any one of embodiments a-i wherein the XRPD pattern includes a peak at 21.2+/−0.4°2theta.
n. In certain embodiments S-5-MBPB Pattern 9A is characterized by any one of embodiments a-i wherein the XRPD pattern includes a peak at 21.7+/−0.4°2theta.
o. In certain embodiments S-5-MBPB Pattern 9A is characterized by any one of embodiments a-i wherein the XRPD pattern includes a peak at 25.3+/−0.4°2theta.
p. In certain embodiments S-5-MBPB Pattern 9A is characterized by any one of embodiments a-o wherein the peaks are within +/−0.3°2theta.
q. In certain embodiments S-5-MBPB Pattern 9A is characterized by any one of embodiments a-o wherein the peaks are within +/−0.2° 2theta.
r. In certain embodiments S-5-MBPB Pattern 9A is characterized by any one of embodiments a-q wherein at least three of the recited peaks have a relative peak intensity of at least 10%.
s. In certain embodiments S-5-MBPB Pattern 9A is characterized by any one of embodiments a-q wherein at least three of the recited peaks have a relative peak intensity of at
t. In certain embodiments S-5-MBPB Pattern 9A is characterized by any one of embodiments a-q wherein at least four of the recited peaks have a relative peak intensity of at least 10%.
u. In certain embodiments S-5-MBPB Pattern 9A is characterized by any one of embodiments a-q wherein at least four of the recited peaks have a relative peak intensity of at least 20%.
v. In certain embodiments S-5-MBPB Pattern 9A is characterized by any one of embodiments a-q wherein at least five of the recited peaks have a relative peak intensity of at least 10%.
w. In certain embodiments S-5-MBPB Pattern 9A is characterized by any one of embodiments a-q wherein at least six of the recited peaks have a relative peak intensity of at least 20%.

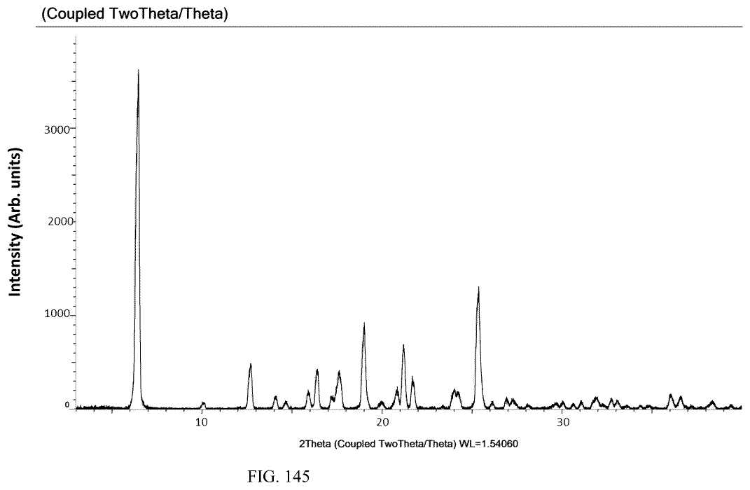

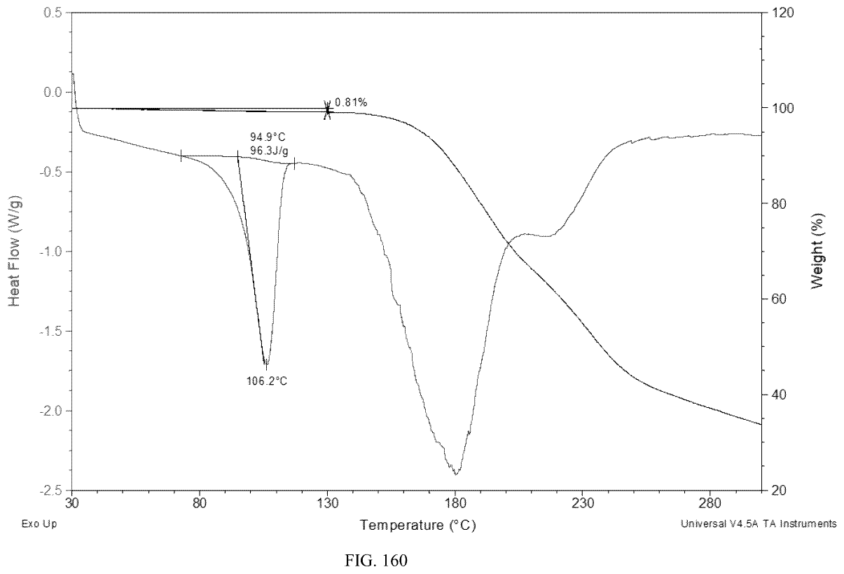

In certain embodiments S-5-MBPB Pattern 9A is characterized by the XRPD diffractogram in FIG. 145 and/or the DSC graph shown in FIG. 160.

### Fumaric Salt

In certain aspects the invention provides S-5-MBPB as fumaric salt. In certain embodiments the S-5-MBPB salt is a stable morphic form denoted Pattern 10A.
a. In certain embodiments S-5-MBPB Pattern 10A is characterized by an XRPD pattern with three or more peaks selected from 11.2, 11.7, 13.0, 13.1, 14.9, 15.8, 17.1, 18.7, 19.7, 20.6, 22.5, 22.7, 24.5, 25.3, 25.6, 26.0, 27.0, and 27.8+/−0.4°2theta.
b. In certain embodiments S-5-MBPB Pattern 10A is characterized by an XRPD pattern with four or more peaks selected from 11.2, 11.7, 13.0, 13.1, 14.9, 15.8, 17.1, 18.7, 19.7, 20.6, 22.5, 22.7, 24.5, 25.3, 25.6, 26.0, 27.0, and 27.8+/−0.4°2theta.
c. In certain embodiments S-5-MBPB Pattern 10A is characterized by an XRPD pattern with five or more peaks selected from 11.2, 11.7, 13.0, 13.1, 14.9, 15.8, 17.1, 18.7, 19.7, 20.6, 22.5, 22.7, 24.5, 25.3, 25.6, 26.0, 27.0, and 27.8+/−0.4° 2theta.
d. In certain embodiments S-5-MBPB Pattern 10A is characterized by an XRPD pattern with six or more peaks selected from 11.2, 11.7, 13.0, 13.1, 14.9, 15.8, 17.1, 18.7, 19.7, 20.6, 22.5, 22.7, 24.5, 25.3, 25.6, 26.0, 27.0, and 27.8+/−0.4°2theta.
e. In certain embodiments S-5-MBPB Pattern 10A is characterized by an XRPD pattern with seven or more peaks selected from 11.2, 11.7, 13.0, 13.1, 14.9, 15.8, 17.1, 18.7, 19.7, 20.6, 22.5, 22.7, 24.5, 25.3, 25.6, 26.0, 27.0, and 27.8+/−0.4°2theta.
f. In certain embodiments S-5-MBPB Pattern 10A is characterized by any one of embodiments a-e wherein the XRPD pattern includes a peak at 11.7+/−0.4°2theta.
g. In certain embodiments S-5-MBPB Pattern 10A is characterized by any one of embodiments a-f wherein the XRPD pattern includes a peak at 13.0+/−0.4°2theta.
h. In certain embodiments S-5-MBPB Pattern 10A is characterized by any one of embodiments a-g wherein the XRPD pattern includes a peak at 13.1+/−0.4° 2theta.
i. In certain embodiments S-5-MBPB Pattern 10A is characterized by any one of embodiments a-h wherein the XRPD pattern includes a peak at 14.9+/−0.4°2theta.
j. In certain embodiments S-5-MBPB Pattern 10A is characterized by any one of embodiments a-i wherein the XRPD pattern includes a peak at 17.1+/−0.4°2theta.
k. In certain embodiments S-5-MBPB Pattern 10A is characterized by any one of embodiments a-i wherein the XRPD pattern includes a peak at 19.7+/−0.4°2theta.
l. In certain embodiments S-5-MBPB Pattern 10A is characterized by any one of embodiments a-i wherein the XRPD pattern includes a peak at 20.6+/−0.4°2theta.
m. In certain embodiments S-5-MBPB Pattern 10A is characterized by any one of embodiments a-i wherein the XRPD pattern includes a peak at 22.7+/−0.4° 2theta.
n. In certain embodiments S-5-MBPB Pattern 10A is characterized by any one of embodiments a-i wherein the XRPD pattern includes a peak at 24.5+/−0.4°2theta.
o. In certain embodiments S-5-MBPB Pattern 10A is characterized by any one of embodiments a-i wherein the XRPD pattern includes a peak at 25.3+/−0.4°2theta.
p. In certain embodiments S-5-MBPB Pattern 10A is characterized by any one of embodiments a-o wherein the peaks are within +/−0.3°2theta.
q. In certain embodiments S-5-MBPB Pattern 10A is characterized by any one of embodiments a-o wherein the peaks are within +/−0.2° 2theta.
r. In certain embodiments S-5-MBPB Pattern 10A is characterized by any one of embodiments a-q wherein at least three of the recited peaks have a relative peak intensity of at least 10%.
s. In certain embodiments S-5-MBPB Pattern 10A is characterized by any one of embodiments a-q wherein at least three of the recited peaks have a relative peak intensity of at
t. In certain embodiments S-5-MBPB Pattern 10A is characterized by any one of embodiments a-q wherein at least four of the recited peaks have a relative peak intensity of at least 10%.
u. In certain embodiments S-5-MBPB Pattern 10A is characterized by any one of embodiments a-q wherein at least four of the recited peaks have a relative peak intensity of at least 20%.
v. In certain embodiments S-5-MBPB Pattern 10A is characterized by any one of embodiments a-q wherein at least five of the recited peaks have a relative peak intensity of at least 10%.
w. In certain embodiments S-5-MBPB Pattern 10A is characterized by any one of embodiments a-q wherein at least six of the recited peaks have a relative peak intensity of at least 20%.

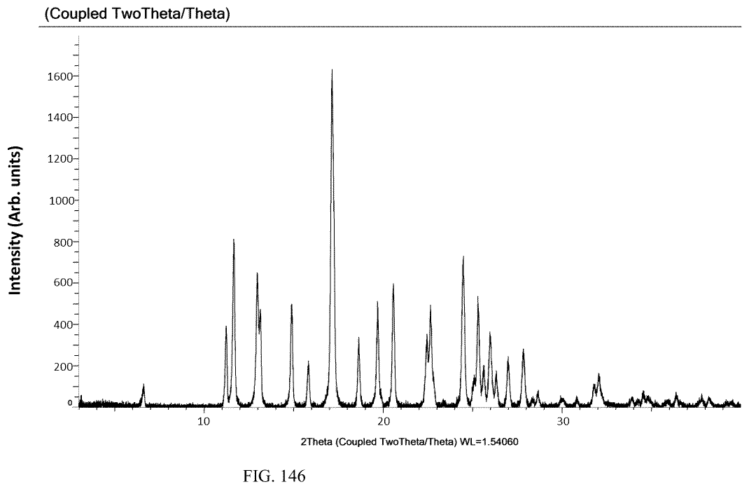

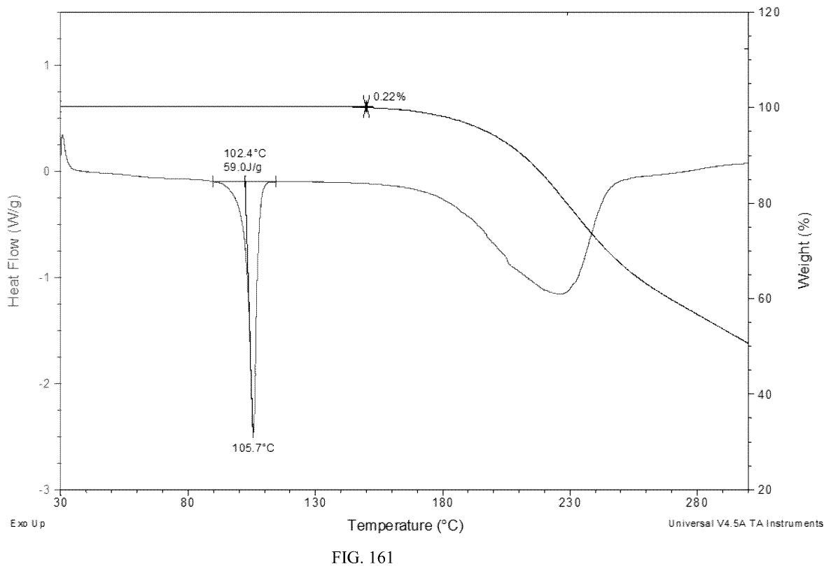

In certain embodiments S-5-MBPB Pattern 10A is characterized by the XRPD diffractogram in FIG. 146 and/or the DSC graph shown in FIG. 161.

### Morphic Forms of R-5-MBPB

### HCl Salt

In certain aspects the invention provides R-5-MBPB as HCl salt. In certain embodiments the R-5-MBPB salt is a stable morphic form denoted Pattern 1A.
a. In certain embodiments R-5-MBPB Pattern 1A is characterized by an XRPD pattern with three or more peaks selected from 5.9, 8.1, 11.7, 13.7, 14.4, 16.3, 17.5, 18.2, 18.8, 19.2, 19.9, 20.8, 21.0, 21.9, 22.3, 23.3, 23.9, 24.6, 25.4, 25.5, 25.7, 27.2, 275, 28.0, 28.8, and 30.3+/−0.4°2theta.
b. In certain embodiments R-5-MBPB Pattern 1A is characterized by an XRPD pattern with four or more peaks selected from 5.9, 8.1, 11.7, 13.7, 14.4, 16.3, 17.5, 18.2, 18.8, 19.2, 19.9, 20.8, 21.0, 21.9, 22.3, 23.3, 23.9, 24.6, 25.4, 25.5, 25.7, 27.2, 275, 28.0, 28.8, and 30.3+/−0.4°2theta.
c. In certain embodiments R-5-MBPB Pattern 1A is characterized by an XRPD pattern with five or more peaks selected from 5.9, 8.1, 11.7, 13.7, 14.4, 16.3, 17.5, 18.2, 18.8, 19.2, 19.9, 20.8, 21.0, 21.9, 22.3, 23.3, 23.9, 24.6, 25.4, 25.5, 25.7, 27.2, 275, 28.0, 28.8, and 30.3+/−0.4°2theta.
d. In certain embodiments R-5-MBPB Pattern 1A is characterized by an XRPD pattern with six or more peaks selected from 5.9, 8.1, 11.7, 13.7, 14.4, 16.3, 17.5, 18.2, 18.8, 19.2, 19.9, 20.8, 21.0, 21.9, 22.3, 23.3, 23.9, 24.6, 25.4, 25.5, 25.7, 27.2, 275, 28.0, 28.8, and 30.3+/−0.4°2theta.
e. In certain embodiments R-5-MBPB Pattern 1A is characterized by an XRPD pattern with seven or more peaks selected from 5.9, 8.1, 11.7, 13.7, 14.4, 16.3, 17.5, 18.2, 18.8, 19.2, 19.9, 20.8, 21.0, 21.9, 22.3, 23.3, 23.9, 24.6, 25.4, 25.5, 25.7, 27.2, 275, 28.0, 28.8, and 30.3+/−0.4° 2theta.
f. In certain embodiments R-5-MBPB Pattern 1A is characterized by any one of embodiments a-e wherein the XRPD pattern includes a peak at 5.9+/−0.4° 2theta.
g. In certain embodiments R-5-MBPB Pattern 1A is characterized by any one of embodiments a-f wherein the XRPD pattern includes a peak at 13.7+/−0.4°2theta.
h. In certain embodiments R-5-MBPB Pattern 1A is characterized by any one of embodiments a-g wherein the XRPD pattern includes a peak at 14.4+/−0.4°2theta.
i. In certain embodiments R-5-MBPB Pattern 1A is characterized by any one of embodiments a-h wherein the XRPD pattern includes a peak at 17.5+/−0.4°2theta.
j. In certain embodiments R-5-MBPB Pattern 1A is characterized by any one of embodiments a-i wherein the XRPD pattern includes a peak at 18.2+/−0.4° 2theta.
k. In certain embodiments R-5-MBPB Pattern 1A is characterized by any one of embodiments a-i wherein the XRPD pattern includes a peak at 18.8+/−0.4°2theta.
l. In certain embodiments R-5-MBPB Pattern 1A is characterized by any one of embodiments a-i wherein the XRPD pattern includes a peak at 19.9+/−0.4°2theta.
m. In certain embodiments R-5-MBPB Pattern 1A is characterized by any one of embodiments a-i wherein the XRPD pattern includes a peak at 21.0+/−0.4°2theta.
n. In certain embodiments R-5-MBPB Pattern 1A is characterized by any one of embodiments a-i wherein the XRPD pattern includes a peak at 23.3+/−0.4°2theta.
o. In certain embodiments R-5-MBPB Pattern 1A is characterized by any one of embodiments a-i wherein the XRPD pattern includes a peak at 25.4+/−0.4° 2theta.
p. In certain embodiments R-5-MBPB Pattern 1A is characterized by any one of embodiments a-o wherein the peaks are within +/−0.3°2theta.
q. In certain embodiments R-5-MBPB Pattern 1A is characterized by any one of embodiments a-o wherein the peaks are within +/−0.2°2theta.
r. In certain embodiments R-5-MBPB Pattern 1A is characterized by any one of embodiments a-q wherein at least three of the recited peaks have a relative peak intensity of at least 10%.
s. In certain embodiments R-5-MBPB Pattern 1A is characterized by any one of embodiments a-q wherein at least three of the recited peaks have a relative peak intensity of at least 20%.
t. In certain embodiments R-5-MBPB Pattern 1A is characterized by any one of embodiments a-q wherein at least four of the recited peaks have a relative peak intensity of at least 10%.
u. In certain embodiments R-5-MBPB Pattern 1A is characterized by any one of embodiments a-q wherein at least four of the recited peaks have a relative peak intensity of at least 20%.
v. In certain embodiments R-5-MBPB Pattern 1A is characterized by any one of embodiments a-q wherein at least five of the recited peaks have a relative peak intensity of at least 10%.
w. In certain embodiments R-5-MBPB Pattern 1A is characterized by any one of embodiments a-q wherein at least six of the recited peaks have a relative peak intensity of at least 20%.

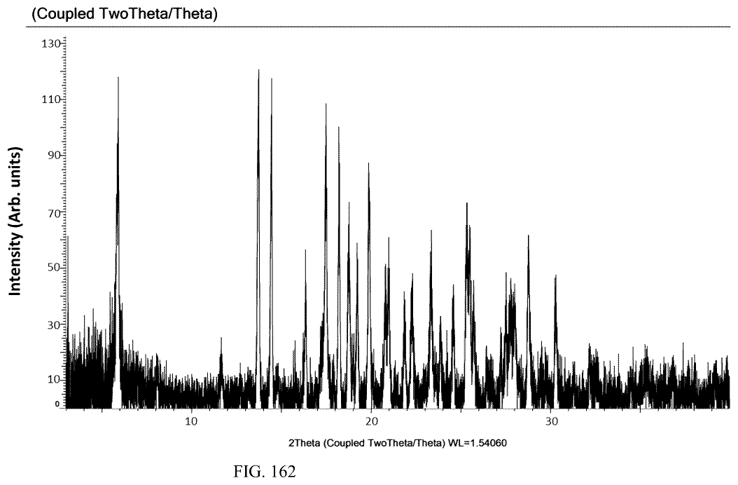

In certain embodiments R-5-MBPB Pattern 1A is characterized by the XRPD diffractogram in FIG. 162.

H₃PO₄ Salt

In certain aspects the invention provides R-5-MBPB as H₃PO₄ salt. In certain embodiments the R-5-MBPB salt is a stable morphic form denoted Pattern 3A.
a. In certain embodiments R-5-MBPB Pattern 3A is characterized by an XRPD pattern with three or more peaks selected from 6.4, 6.5, 12.7, 12.8, 17.1, 19.1, 20.9, 21.3, 21.8, and 26.5+/−0.4° 2theta.
b. In certain embodiments R-5-MBPB Pattern 3A is characterized by an XRPD pattern with four or more peaks selected from 6.4, 6.5, 12.7, 12.8, 17.1, 19.1, 20.9, 21.3, 21.8, and 26.5+/−0.4° 2theta.
c. In certain embodiments R-5-MBPB Pattern 3A is characterized by an XRPD pattern with five or more peaks selected from 6.4, 6.5, 12.7, 12.8, 17.1, 19.1, 20.9, 21.3, 21.8, and 26.5+/−0.4°2theta.
d. In certain embodiments R-5-MBPB Pattern 3A is characterized by an XRPD pattern with six or more peaks selected from 6.4, 6.5, 12.7, 12.8, 17.1, 19.1, 20.9, 21.3, 21.8, and 26.5+/−0.4° 2theta.
e. In certain embodiments R-5-MBPB Pattern 3A is characterized by an XRPD pattern with seven or more peaks selected from 6.4, 6.5, 12.7, 12.8, 17.1, 19.1, 20.9, 21.3, 21.8, and 26.5+/−0.4° 2theta.
f. In certain embodiments R-5-MBPB Pattern 3A is characterized by any one of embodiments a-e wherein the XRPD pattern includes a peak at 6.4+/−0.4° 2theta.
g. In certain embodiments R-5-MBPB Pattern 3A is characterized by any one of embodiments a-f wherein the XRPD pattern includes a peak at 6.7+/−0.4°2theta.
h. In certain embodiments R-5-MBPB Pattern 3A is characterized by any one of embodiments a-g wherein the XRPD pattern includes a peak at 12.7+/−0.4°2theta.
i. In certain embodiments R-5-MBPB Pattern 3A is characterized by any one of embodiments a-h wherein the XRPD pattern includes a peak at 13.1+/−0.4°2theta.
j. In certain embodiments R-5-MBPB Pattern 3A is characterized by any one of embodiments a-i wherein the XRPD pattern includes a peak at 17.1+/−0.4°2theta.
k. In certain embodiments R-5-MBPB Pattern 3A is characterized by any one of embodiments a-i wherein the XRPD pattern includes a peak at 19.1+/−0.4°2theta.
l. In certain embodiments R-5-MBPB Pattern 3A is characterized by any one of embodiments a-i wherein the XRPD pattern includes a peak at 20.9+/−0.4°2theta.
m. In certain embodiments R-5-MBPB Pattern 3A is characterized by any one of embodiments a-i wherein the XRPD pattern includes a peak at 21.3+/−0.4°2theta.
n. In certain embodiments R-5-MBPB Pattern 3A is characterized by any one of embodiments a-i wherein the XRPD pattern includes a peak at 21.8+/−0.4°2theta.
o. In certain embodiments R-5-MBPB Pattern 3A is characterized by any one of embodiments a-i wherein the XRPD pattern includes a peak at 26.5+/−0.4°2theta.
p. In certain embodiments R-5-MBPB Pattern 3A is characterized by any one of embodiments a-o wherein the peaks are within +/−0.3°2theta.
q. In certain embodiments R-5-MBPB Pattern 3A is characterized by any one of embodiments a-o wherein the peaks are within +/−0.2°2theta.
r. In certain embodiments R-5-MBPB Pattern 3A is characterized by any one of embodiments a-q wherein at least three of the recited peaks have a relative peak intensity of at least 10%.
s. In certain embodiments R-5-MBPB Pattern 3A is characterized by any one of embodiments a-q wherein at least three of the recited peaks have a relative peak intensity of at least 20%.
t. In certain embodiments R-5-MBPB Pattern 3A is characterized by any one of embodiments a-q wherein at least four of the recited peaks have a relative peak intensity of at least 10%.
u. In certain embodiments R-5-MBPB Pattern 3A is characterized by any one of embodiments a-q wherein at least four of the recited peaks have a relative peak intensity of at least 20%.
v. In certain embodiments R-5-MBPB Pattern 3A is characterized by any one of embodiments a-q wherein at least five of the recited peaks have a relative peak intensity of at least 10%.
w. In certain embodiments R-5-MBPB Pattern 3A is characterized by any one of embodiments a-q wherein at least six of the recited peaks have a relative peak intensity of at least 20%.

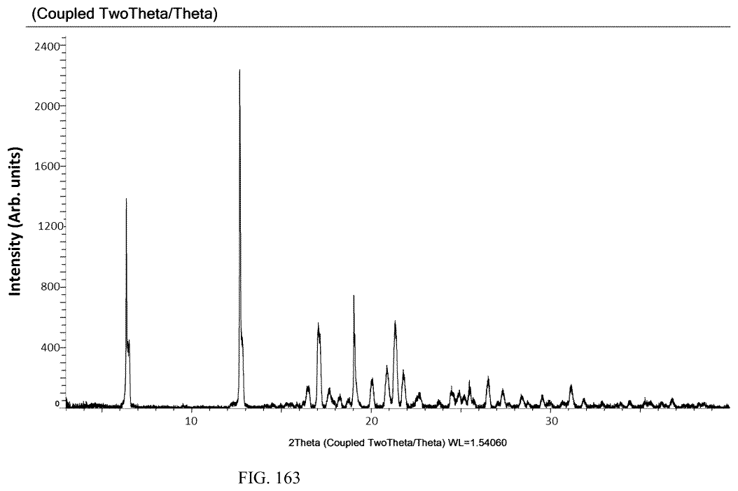

In certain embodiments R-5-MBPB Pattern 3A is characterized by the XRPD diffractogram in FIG. 163.

### Maleic Salt

In certain aspects the invention provides R-5-MBPB as maleic salt. In certain embodiments the R-5-MBPB salt is a stable morphic form denoted Pattern 8A.
a. In certain embodiments R-5-MBPB Pattern 8A is characterized by an XRPD pattern with three or more peaks selected from 5.9, 10.0, 12.8, 14.9, 17.0, 17.1, 19.7, 22.1, 22.7, 22.8, 23.5, 24.7, 25.4, 27.5, 28.5, 28.6, and 28.9+/−0.4°2theta.
b. In certain embodiments R-5-MBPB Pattern 8A is characterized by an XRPD pattern with four or more peaks selected from 5.9, 10.0, 12.8, 14.9, 17.0, 17.1, 19.7, 22.1, 22.7, 22.8, 23.5, 24.7, 25.4, 27.5, 28.5, 28.6, and 28.9+/−0.4°2theta.
c. In certain embodiments R-5-MBPB Pattern 8A is characterized by an XRPD pattern with five or more peaks selected from 5.9, 10.0, 12.8, 14.9, 17.0, 17.1, 19.7, 22.1, 22.7, 22.8, 23.5, 24.7, 25.4, 27.5, 28.5, 28.6, and 28.9+/−0.4°2theta.
d. In certain embodiments R-5-MBPB Pattern 8A is characterized by an XRPD pattern with six or more peaks selected from 5.9, 10.0, 12.8, 14.9, 17.0, 17.1, 19.7, 22.1, 22.7, 22.8, 23.5, 24.7, 25.4, 27.5, 28.5, 28.6, and 28.9+/−0.4°2theta.
e. In certain embodiments R-5-MBPB Pattern 8A is characterized by an XRPD pattern with seven or more peaks selected from 5.9, 10.0, 12.8, 14.9, 17.0, 17.1, 19.7, 22.1, 22.7, 22.8, 23.5, 24.7, 25.4, 27.5, 28.5, 28.6, and 28.9+/−0.4° 2theta.
f. In certain embodiments R-5-MBPB Pattern 8A is characterized by any one of embodiments a-e wherein the XRPD pattern includes a peak at 10.0+/−0.4° 2theta.
g. In certain embodiments R-5-MBPB Pattern 8A is characterized by any one of embodiments a-f wherein the XRPD pattern includes a peak at 12.8+/−0.4° 2theta.
h. In certain embodiments R-5-MBPB Pattern 8A is characterized by any one of embodiments a-g wherein the XRPD pattern includes a peak at 14.9+/−0.4°2theta.
i. In certain embodiments R-5-MBPB Pattern 8A is characterized by any one of embodiments a-h wherein the XRPD pattern includes a peak at 17.0+/−0.4°2theta.
j. In certain embodiments R-5-MBPB Pattern 8A is characterized by any one of embodiments a-i wherein the XRPD pattern includes a peak at 17.4+/−0.4°2theta.
k. In certain embodiments R-5-MBPB Pattern 8A is characterized by any one of embodiments a-i wherein the XRPD pattern includes a peak at 19.7+/−0.4°2theta.
l. In certain embodiments R-5-MBPB Pattern 8A is characterized by any one of embodiments a-i wherein the XRPD pattern includes a peak at 22.1+/−0.4° 2theta.
m. In certain embodiments R-5-MBPB Pattern 8A is characterized by any one of embodiments a-i wherein the XRPD pattern includes a peak at 22.7+/−0.4°2theta.
n. In certain embodiments R-5-MBPB Pattern 8A is characterized by any one of embodiments a-i wherein the XRPD pattern includes a peak at 27.5+/−0.4°2theta.
o. In certain embodiments R-5-MBPB Pattern 8A is characterized by any one of embodiments a-i wherein the XRPD pattern includes a peak at 28.5+/−0.4°2theta.
p. In certain embodiments R-5-MBPB Pattern 8A is characterized by any one of embodiments a-o wherein the peaks are within +/−0.3°2theta.
q. In certain embodiments R-5-MBPB Pattern 8A is characterized by any one of embodiments a-o wherein the peaks are within +/−0.2°2theta.
r. In certain embodiments R-5-MBPB Pattern 8A is characterized by any one of embodiments a-q wherein at least three of the recited peaks have a relative peak intensity of at least 10%.
s. In certain embodiments R-5-MBPB Pattern 8A is characterized by any one of embodiments a-q wherein at least three of the recited peaks have a relative peak intensity of at least 20%.
t. In certain embodiments R-5-MBPB Pattern 8A is characterized by any one of embodiments a-q wherein at least four of the recited peaks have a relative peak intensity of at least 10%.
u. In certain embodiments R-5-MBPB Pattern 8A is characterized by any one of embodiments a-q wherein at least four of the recited peaks have a relative peak intensity of at least 20%.
v. In certain embodiments R-5-MBPB Pattern 8A is characterized by any one of embodiments a-q wherein at least five of the recited peaks have a relative peak intensity of at least 10%.
w. In certain embodiments R-5-MBPB Pattern 8A is characterized by any one of embodiments a-q wherein at least six of the recited peaks have a relative peak intensity of at least 20%.

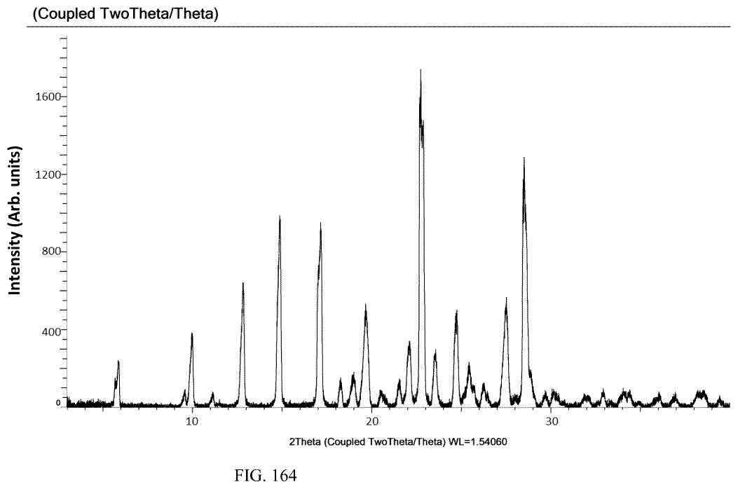

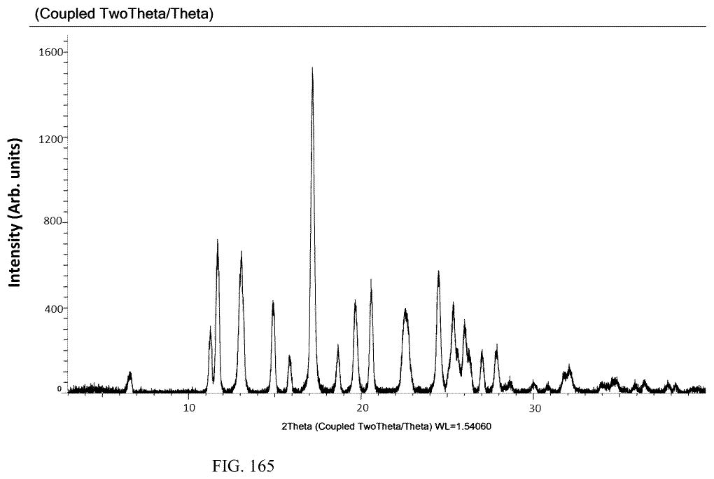

In certain embodiments R-5-MBPB Pattern 8A is characterized by the XRPD diffractogram in FIG. 164.

In certain aspects the invention provides R-5-MBPB as fumaric salt. In certain embodiments the R-5-MBPB salt is a stable morphic form denoted Pattern 10A.
a. In certain embodiments R-5-MBPB Pattern 10A is characterized by an XRPD pattern with three or more peaks selected from 11.3, 11.7, 13.1, 14.9, 15.9, 17.2, 18.7, 19.7, 20.6, 22.6, 24.5, 25.4, 25.6, 26.0, 27.0, and 27.9+/−0.4° 2theta.
b. In certain embodiments R-5-MBPB Pattern 10A is characterized by an XRPD pattern with four or more peaks selected from 11.3, 11.7, 13.1, 14.9, 15.9, 17.2, 18.7, 19.7, 20.6, 22.6, 24.5, 25.4, 25.6, 26.0, 27.0, and 27.9+/−0.4°2theta.
c. In certain embodiments R-5-MBPB Pattern 10A is characterized by an XRPD pattern with five or more peaks selected from 11.3, 11.7, 13.1, 14.9, 15.9, 17.2, 18.7, 19.7, 20.6, 22.6, 24.5, 25.4, 25.6, 26.0, 27.0, and 27.9+/−0.4°2theta.
d. In certain embodiments R-5-MBPB Pattern 10A is characterized by an XRPD pattern with six or more peaks selected from 11.3, 11.7, 13.1, 14.9, 15.9, 17.2, 18.7, 19.7, 20.6, 22.6, 24.5, 25.4, 25.6, 26.0, 27.0, and 27.9+/−0.4° 2theta.
e. In certain embodiments R-5-MBPB Pattern 10A is characterized by an XRPD pattern with seven or more peaks selected from 11.3, 11.7, 13.1, 14.9, 15.9, 17.2, 18.7, 19.7, 20.6, 22.6, 24.5, 25.4, 25.6, 26.0, 27.0, and 27.9+/−0.4°2theta.
f. In certain embodiments R-5-MBPB Pattern 10A is characterized by any one of embodiments a-e wherein the XRPD pattern includes a peak at 11.3+/−0.4° 2theta.
g. In certain embodiments R-5-MBPB Pattern 10A is characterized by any one of embodiments a-f wherein the XRPD pattern includes a peak at 11.7+/−0.4°2theta.
h. In certain embodiments R-5-MBPB 3 Pattern 10A is characterized by any one of embodiments a-g wherein the XRPD pattern includes a peak at 13.1+/−0.4°2theta.
i. In certain embodiments R-5-MBPB Pattern 10A is characterized by any one of embodiments a-h wherein the XRPD pattern includes a peak at 14.9+/−0.4°2theta.
j. In certain embodiments R-5-MBPB Pattern 10A is characterized by any one of embodiments a-i wherein the XRPD pattern includes a peak at 17.2+/−0.4° 2theta.
k. In certain embodiments R-5-MBPB Pattern 10A is characterized by any one of embodiments a-i wherein the XRPD pattern includes a peak at 17.6+/−0.4°2theta.
l. In certain embodiments R-5-MBPB Pattern 10A is characterized by any one of embodiments a-i wherein the XRPD pattern includes a peak at 19.7+/−0.4°2theta.
m. In certain embodiments R-5-MBPB Pattern 10A is characterized by any one of embodiments a-i wherein the XRPD pattern includes a peak at 21.0+/−0.4°2theta.
n. In certain embodiments R-5-MBPB Pattern 10A is characterized by any one of embodiments a-i wherein the XRPD pattern includes a peak at 24.9+/−0.4° 2theta.
o. In certain embodiments R-5-MBPB Pattern 10A is characterized by any one of embodiments a-i wherein the XRPD pattern includes a peak at 25.8+/−0.4° 2theta.
p. In certain embodiments R-5-MBPB Pattern 10A is characterized by any one of embodiments a-o wherein the peaks are within +/−0.3°2theta.
q. In certain embodiments R-5-MBPB Pattern 10A is characterized by any one of embodiments a-o wherein the peaks are within +/−0.2° 2theta.
r. In certain embodiments R-5-MBPB Pattern 10A is characterized by any one of embodiments a-q wherein at least three of the recited peaks have a relative peak intensity of at least 10%.
s. In certain embodiments R-5-MBPB Pattern 10A is characterized by any one of embodiments a-q wherein at least three of the recited peaks have a relative peak intensity of at least 20%.
t. In certain embodiments R-5-MBPB Pattern 10A is characterized by any one of embodiments a-q wherein at least four of the recited peaks have a relative peak intensity of at least 10%.
u. In certain embodiments R-5-MBPB Pattern 10A is characterized by any one of embodiments a-q wherein at least four of the recited peaks have a relative peak intensity of at least 20%.
v. In certain embodiments R-5-MBPB Pattern 10A is characterized by any one of embodiments a-q wherein at least five of the recited peaks have a relative peak intensity of at least 10%.
w. In certain embodiments R-5-MBPB Pattern 10A is characterized by any one of embodiments a-q wherein at least six of the recited peaks have a relative peak intensity of at least 20%.

In certain embodiments R-5-MBPB Pattern 10A is characterized by the XRPD diffractogram in FIG. 165.

### Morphic Forms of R-6-MBPB

### HCl Salt

In certain aspects the invention provides R-6-MBPB as HCl salt. In certain embodiments the R-6-MBPB salt is a stable morphic form denoted Pattern 1A.
a. In certain embodiments R-6-MBPB Pattern 1A is characterized by an XRPD pattern with three or more peaks selected from 13.9, 14.0, 15.6, 16.8, 17.7, 18.4, 184, 20.6, 22.1, 22.2, 23.9, 24.7, 25.0, 26.7, 27.8, 27.9, 28.1, and 28.3+/−0.4° 2theta.
b. In certain embodiments R-6-MBPB Pattern 1A is characterized by an XRPD pattern with four or more peaks selected from 13.9, 14.0, 15.6, 16.8, 17.7, 18.4, 184, 20.6, 22.1, 22.2, 23.9, 24.7, 25.0, 26.7, 27.8, 27.9, 28.1, and 28.3+/−0.4° 2theta.
c. In certain embodiments R-6-MBPB Pattern 1A is characterized by an XRPD pattern with five or more peaks selected from 13.9, 14.0, 15.6, 16.8, 17.7, 18.4, 184, 20.6, 22.1, 22.2, 23.9, 24.7, 25.0, 26.7, 27.8, 27.9, 28.1, and 28.3+/−0.4° 2theta.
d. In certain embodiments R-6-MBPB Pattern 1A is characterized by an XRPD pattern with six or more peaks selected from 13.9, 14.0, 15.6, 16.8, 17.7, 18.4, 184, 20.6, 22.1, 22.2, 23.9, 24.7, 25.0, 26.7, 27.8, 27.9, 28.1, and 28.3+/−0.4°2theta.
e. In certain embodiments R-6-MBPB Pattern 1A is characterized by an XRPD pattern with seven or more peaks selected from 13.9, 14.0, 15.6, 16.8, 17.7, 18.4, 184, 20.6, 22.1, 22.2, 23.9, 24.7, 25.0, 26.7, 27.8, 27.9, 28.1, and 28.3+/−0.4° 2theta.
f. In certain embodiments R-6-MBPB Pattern 1A is characterized by any one of embodiments a-e wherein the XRPD pattern includes a peak at 13.9+/−0.4°2theta.
g. In certain embodiments R-6-MBPB Pattern 1A is characterized by any one of embodiments a-f wherein the XRPD pattern includes a peak at 14.3+/−0.4°2theta.
h. In certain embodiments R-6-MBPB Pattern 1A is characterized by any one of embodiments a-g wherein the XRPD pattern includes a peak at 15.6+/−0.4°2theta.
i. In certain embodiments R-6-MBPB Pattern 1A is characterized by any one of embodiments a-h wherein the XRPD pattern includes a peak at 17.9+/−0.4°2theta.
j. In certain embodiments R-6-MBPB Pattern 1A is characterized by any one of embodiments a-i wherein the XRPD pattern includes a peak at 18.4+/−0.4°2theta.
k. In certain embodiments R-6-MBPB Pattern 1A is characterized by any one of embodiments a-i wherein the XRPD pattern includes a peak at 19.0+/−0.4°2theta.
l. In certain embodiments R-6-MBPB Pattern 1A is characterized by any one of embodiments a-i wherein the XRPD pattern includes a peak at 24.7+/−0.4° 2theta.
m. In certain embodiments R-6-MBPB Pattern 1A is characterized by any one of embodiments a-i wherein the XRPD pattern includes a peak at 25.0+/−0.4°2theta.
n. In certain embodiments R-6-MBPB Pattern 1A is characterized by any one of embodiments a-i wherein the XRPD pattern includes a peak at 27.8+/−0.4°2theta.
o. In certain embodiments R-6-MBPB Pattern 1A is characterized by any one of embodiments a-i wherein the XRPD pattern includes a peak at 28.3+/−0.4°2theta.
p. In certain embodiments R-6-MBPB Pattern 1A is characterized by any one of embodiments a-o wherein the peaks are within +/−0.3°2theta.
q. In certain embodiments R-6-MBPB Pattern 1A is characterized by any one of embodiments a-o wherein the peaks are within +/−0.2°2theta.
r. In certain embodiments R-6-MBPB Pattern 1A is characterized by any one of embodiments a-q wherein at least three of the recited peaks have a relative peak intensity of at least 10%.
s. In certain embodiments R-6-MBPB Pattern 1A is characterized by any one of embodiments a-q wherein at least three of the recited peaks have a relative peak intensity of at least 20%.
t. In certain embodiments R-6-MBPB Pattern 1A is characterized by any one of embodiments a-q wherein at least four of the recited peaks have a relative peak intensity of at least 10%.
u. In certain embodiments R-6-MBPB Pattern 1A is characterized by any one of embodiments a-q wherein at least four of the recited peaks have a relative peak intensity of at least 20%.
v. In certain embodiments R-6-MBPB Pattern 1A is characterized by any one of embodiments a-q wherein at least five of the recited peaks have a relative peak intensity of at least 10%.
w. In certain embodiments R-6-MBPB Pattern 1A is characterized by any one of embodiments a-q wherein at least six of the recited peaks have a relative peak intensity of at least 20%.

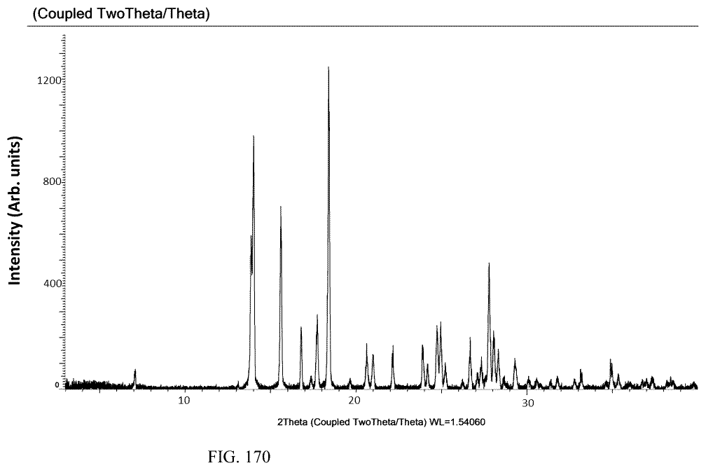

In certain embodiments R-6-MBPB Pattern 1A is characterized by the XRPD diffractogram in FIG. 170.

### HBr Salt

In certain aspects the invention provides R-6-MBPB as HBr salt. In certain embodiments the R-6-MBPB salt is a stable morphic form denoted Pattern 2A.
a. In certain embodiments R-6-MBPB Pattern 2A is characterized by an XRPD pattern with three or more peaks selected from 7.1, 7.3, 13.6, 14.4, 15.8, 18.4, 21.6, 23.1, 23.4, 24.1, 24.2, 24.3, 24.7, 26.3, 26.4, 27.2, 27.3, 28.8, and 33.7+/−0.4°2theta.
b. In certain embodiments R-6-MBPB Pattern 2A is characterized by an XRPD pattern with four or more peaks selected from 7.1, 7.3, 13.6, 14.4, 15.8, 18.4, 21.6, 23.1, 23.4, 24.1, 24.2, 24.3, 24.7, 26.3, 26.4, 27.2, 27.3, 28.8, and 33.7+/−0.4°2theta.
c. In certain embodiments R-6-MBPB Pattern 2A is characterized by an XRPD pattern with five or more peaks selected from 7.1, 7.3, 13.6, 14.4, 15.8, 18.4, 21.6, 23.1, 23.4, 24.1, 24.2, 24.3, 24.7, 26.3, 26.4, 27.2, 27.3, 28.8, and 33.7+/−0.4°2theta.
d. In certain embodiments R-6-MBPB Pattern 2A is characterized by an XRPD pattern with six or more peaks selected from 7.1, 7.3, 13.6, 14.4, 15.8, 18.4, 21.6, 23.1, 23.4, 24.1, 24.2, 24.3, 24.7, 26.3, 26.4, 27.2, 27.3, 28.8, and 33.7+/−0.4°2theta.
e. In certain embodiments R-6-MBPB Pattern 2A is characterized by an XRPD pattern with seven or more peaks selected from 7.1, 7.3, 13.6, 14.4, 15.8, 18.4, 21.6, 23.1, 23.4, 24.1, 24.2, 24.3, 24.7, 26.3, 26.4, 27.2, 27.3, 28.8, and 33.7+/−0.4°2theta.
f. In certain embodiments R-6-MBPB Pattern 2A is characterized by any one of embodiments a-e wherein the XRPD pattern includes a peak at 7.3+/−0.4° 2theta.
g. In certain embodiments R-6-MBPB Pattern 2A is characterized by any one of embodiments a-f wherein the XRPD pattern includes a peak at 13.6+/−0.4° 2theta.
h. In certain embodiments R-6-MBPB Pattern 2A is characterized by any one of embodiments a-g wherein the XRPD pattern includes a peak at 14.4+/−0.4°2theta.
i. In certain embodiments R-6-MBPB Pattern 2A is characterized by any one of embodiments a-h wherein the XRPD pattern includes a peak at 15.8+/−0.4°2theta.
j. In certain embodiments R-6-MBPB Pattern 2A is characterized by any one of embodiments a-i wherein the XRPD pattern includes a peak at 18.4+/−0.4°2theta.
k. In certain embodiments R-6-MBPB Pattern 2A is characterized by any one of embodiments a-i wherein the XRPD pattern includes a peak at 23.4+/−0.4° 2theta.
l. In certain embodiments R-6-MBPB Pattern 2A is characterized by any one of embodiments a-i wherein the XRPD pattern includes a peak at 24.3+/−0.4°2theta.
m. In certain embodiments R-6-MBPB Pattern 2A is characterized by any one of embodiments a-i wherein the XRPD pattern includes a peak at 24.9+/−0.4°2theta.
n. In certain embodiments R-6-MBPB Pattern 2A is characterized by any one of embodiments a-i wherein the XRPD pattern includes a peak at 27.2+/−0.4°2theta.
o. In certain embodiments R-6-MBPB Pattern 2A is characterized by any one of embodiments a-i wherein the XRPD pattern includes a peak at 27.8+/−0.4°2theta.
p. In certain embodiments R-6-MBPB Pattern 2A is characterized by any one of embodiments a-o wherein the peaks are within +/−0.3°2theta.
q. In certain embodiments R-6-MBPB Pattern 2A is characterized by any one of embodiments a-o wherein the peaks are within +/−0.2°2theta.
r. In certain embodiments R-6-MBPB Pattern 2A is characterized by any one of embodiments a-q wherein at least three of the recited peaks have a relative peak intensity of at least 10%.
s. In certain embodiments R-6-MBPB Pattern 2A is characterized by any one of embodiments a-q wherein at least three of the recited peaks have a relative peak intensity of at
t. In certain embodiments R-6-MBPB Pattern 2A is characterized by any one of embodiments a-q wherein at least four of the recited peaks have a relative peak intensity of at least 10%.
u. In certain embodiments R-6-MBPB Pattern 2A is characterized by any one of embodiments a-q wherein at least four of the recited peaks have a relative peak intensity of at least 20%.
v. In certain embodiments R-6-MBPB Pattern 2A is characterized by any one of embodiments a-q wherein at least five of the recited peaks have a relative peak intensity of at least 10%.
w. In certain embodiments R-6-MBPB Pattern 2A is characterized by any one of embodiments a-q wherein at least six of the recited peaks have a relative peak intensity of at least 20%.

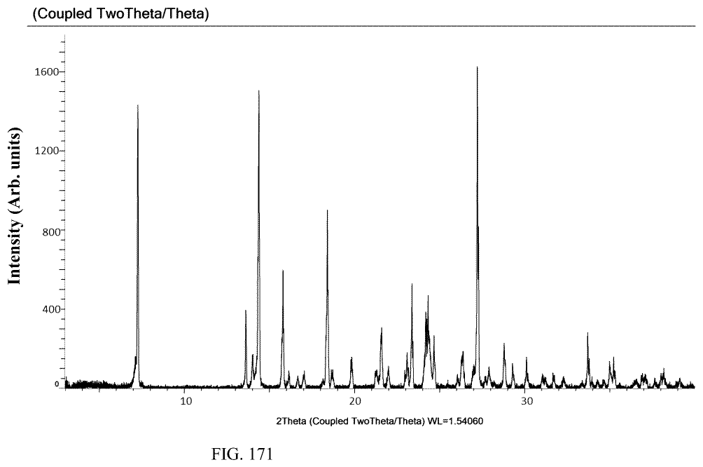

In certain embodiments R-6-MBPB Pattern 2A is characterized by the XRPD diffractogram in FIG. 171.

### Oxalate Salt

In certain aspects the invention provides R-6-MBPB as oxalate salt. In certain embodiments the R-6-MBPB salt is a stable morphic form denoted Pattern 9A.
a. In certain embodiments R-6-MBPB Pattern 9A is characterized by an XRPD pattern with three or more peaks selected from 11.7, 12.4, 15.4, 15.9, 17.0, 19.1, 19.9, 20.9, 21.0, 21.1, 21.6, 25.5, 25.8, 26.7, 32.5, and 32.6+/−0.4° 2theta.
b. In certain embodiments R-6-MBPB Pattern 9A is characterized by an XRPD pattern with four or more peaks selected from 11.7, 12.4, 15.4, 15.9, 17.0, 19.1, 19.9, 20.9, 21.0, 21.1, 21.6, 25.5, 25.8, 26.7, 32.5, and 32.6+/−0.4° 2theta.
c. In certain embodiments R-6-MBPB Pattern 9A is characterized by an XRPD pattern with five or more peaks selected from 11.7, 12.4, 15.4, 15.9, 17.0, 19.1, 19.9, 20.9, 21.0, 21.1, 21.6, 25.5, 25.8, 26.7, 32.5, and 32.6+/−0.4° 2theta.
d. In certain embodiments R-6-MBPB Pattern 9A is characterized by an XRPD pattern with six or more peaks selected from 11.7, 12.4, 15.4, 15.9, 17.0, 19.1, 19.9, 20.9, 21.0, 21.1, 21.6, 25.5, 25.8, 26.7, 32.5, and 32.6+/−0.4°2theta.
e. In certain embodiments R-6-MBPB Pattern 9A is characterized by an XRPD pattern with seven or more peaks selected from 11.7, 12.4, 15.4, 15.9, 17.0, 19.1, 19.9, 20.9, 21.0, 21.1, 21.6, 25.5, 25.8, 26.7, 32.5, and 32.6+/−0.4° 2theta.
f. In certain embodiments R-6-MBPB Pattern 9A is characterized by any one of embodiments a-e wherein the XRPD pattern includes a peak at 11.7+/−0.4° 2theta.
g. In certain embodiments R-6-MBPB Pattern 9A is characterized by any one of embodiments a-f wherein the XRPD pattern includes a peak at 12.4+/−0.4° 2theta.
h. In certain embodiments R-6-MBPB Pattern 9A is characterized by any one of embodiments a-g wherein the XRPD pattern includes a peak at 15.9+/−0.4°2theta.
i. In certain embodiments R-6-MBPB Pattern 9A is characterized by any one of embodiments a-h wherein the XRPD pattern includes a peak at 19.1+/−0.4°2theta.
j. In certain embodiments R-6-MBPB Pattern 9A is characterized by any one of embodiments a-i wherein the XRPD pattern includes a peak at 20.4+/−0.4°2theta.
k. In certain embodiments R-6-MBPB Pattern 9A is characterized by any one of embodiments a-i wherein the XRPD pattern includes a peak at 20.9+/−0.4° 2theta.
l. In certain embodiments R-6-MBPB Pattern 9A is characterized by any one of embodiments a-i wherein the XRPD pattern includes a peak at 21.0+/−0.4°2theta.
m. In certain embodiments R-6-MBPB Pattern 9A is characterized by any one of embodiments a-i wherein the XRPD pattern includes a peak at 21.6+/−0.4°2theta.
n. In certain embodiments R-6-MBPB Pattern 9A is characterized by any one of embodiments a-i wherein the XRPD pattern includes a peak at 26.7+/−0.4° 2theta.
o. In certain embodiments R-6-MBPB Pattern 9A is characterized by any one of embodiments a-i wherein the XRPD pattern includes a peak at 32.5+/−0.4° 2theta.
p. In certain embodiments R-6-MBPB Pattern 9A is characterized by any one of embodiments a-o wherein the peaks are within +/−0.3°2theta.
q. In certain embodiments R-6-MBPB Pattern 9A is characterized by any one of embodiments a-o wherein the peaks are within +/−0.2°2theta.
r. In certain embodiments R-6-MBPB Pattern 9A is characterized by any one of embodiments a-q wherein at least three of the recited peaks have a relative peak intensity of at least 10%.
s. In certain embodiments R-6-MBPB Pattern 9A is characterized by any one of embodiments a-q wherein at least three of the recited peaks have a relative peak intensity of at
t. In certain embodiments R-6-MBPB Pattern 9A is characterized by any one of embodiments a-q wherein at least four of the recited peaks have a relative peak intensity of at least 10%.
u. In certain embodiments R-6-MBPB Pattern 9A is characterized by any one of embodiments a-q wherein at least four of the recited peaks have a relative peak intensity of at least 20%.
v. In certain embodiments R-6-MBPB Pattern 9A is characterized by any one of embodiments a-q wherein at least five of the recited peaks have a relative peak intensity of at least 10%.
w. In certain embodiments R-6-MBPB Pattern 9A is characterized by any one of embodiments a-q wherein at least six of the recited peaks have a relative peak intensity of at least 20%.

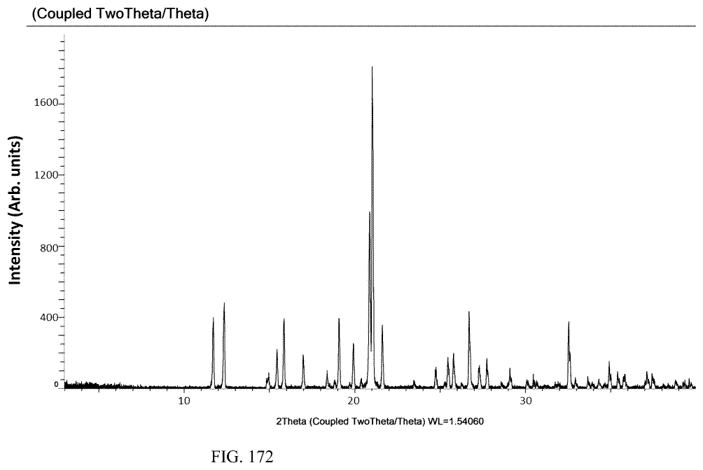

In certain embodiments R-6-MBPB Pattern 9A is characterized by the XRPD diffractogram in FIG. 172.

### Other Compounds

In other embodiments, the invention provides a salt morphic form or a mixture of salts of a benzofuran compound selected from Formula I, Formula II, Formula III, Formula IV, Formula V, Formula VI, Formula VII, Formula VIII, Formula IX, and Formula X:

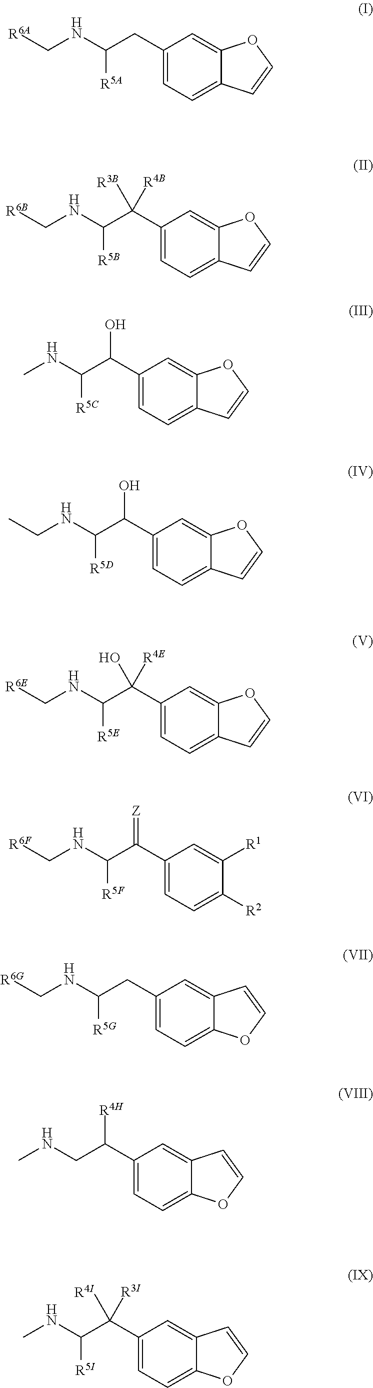

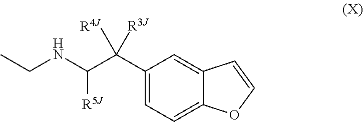

wherein:
   R¹ and R² are taken together as —OCH═CH— or —CH═CHO—;
   R^{3B} and R^{4B} are independently selected from —H, —X, C₁-C₄ alkyl, —CH₂OH, —CH₂X, —CHX₂, and —CX₃, wherein at least one of R^{3B} and R^{4B} is not —H;
   R^{3I} and R^{4I} are independently selected from —H, —X, —OH, —CH₂OH, —CH₂X, —CHX₂, —CX₃, and C₁-C₄ alkyl; wherein at least one of R^{3I} and R^{4I} is not —H;
   R^{3J} and RAJ are independently selected from —H, —X, —OH, C₁-C₄ alkyl, —CH₂OH, —CH₂X, —CHX₂, and —CX₃;
   R^{4E} is selected from C₁-C₄ alkyl, —CH₂OH, —CH₂X, —CHX₂, and —CX₃; R^{4H} is selected from —X, —CH₂CH₂CH₃, —CH₂OH, —CH₂X, and —CHX₂;
   R^{5A} and R^{5G} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl, when R^{5A} is C₂ alkyl or H, R^{6A} is not —H, and when R^{5G} is —H or C₂ alkyl, R^{6G} is not —H;
   R^{5B} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl;
   R^{5C} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
   R^{5D}, R^{5E}, R^{5F}, and R^{5J} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl, when R^{5F} is —H or C₁ alkyl, R^{6F} cannot be —H, and when R^{5J} is C₁ alkyl, at least one of R^{3J} and R^{4J} is not H;
   R^{5I} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; wherein at least one of R^{3I}, R^{4I}, and R^{5I} is not C₁ alkyl;
   R^{6A}, R^{6B}, R^{6E}, R^{6F}, and R^{6G} are independently selected from —H and —CH₃;
   X is independently selected from —F, —C₁, and —Br; and
   Z is selected from O and CH₂.

The salt morphic form, morphic salt mixture, or specified salt mixture described herein of compounds of Formulas I-X can be used as racemic mixtures, enantiomerically or diastereomerically enriched or substantially pure or pure isomers, as desired to achieve the goal of therapy.

In further embodiments, the invention includes salt a morphic form or a mixture of salts of an enantiomerically enriched compound of Formula XI, Formula XII, or Formula XIII or a pharmaceutically acceptable salt or mixed salt thereof:

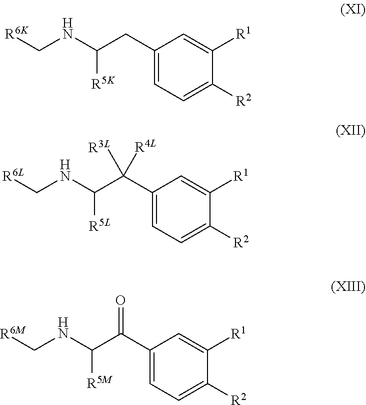

wherein:
   R¹ and R² are taken together as —OCH═CH— or —CH═CHO—;
   R^{3L} and R^{4L} are independently selected from —H, —X, —OH, C₁-C₄ alkyl, —CH₂OH, —CH₂X, —CHX₂, and —CX₃, wherein at least one of R^{3L} and R^{4L} is not —H;
   R^{5K} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
   R^{5L} and R^{5M} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; and
   R^{6K}, R^{6L}, and R^{6M} are selected from —H and —CH₃.

In other embodiments, the present invention provides a salt morphic form, morphic salt mixture, or specified salt mixture of an enantiomerically enriched compound of Formula A, Formula B, Formula C, Formula D, Formula E, or Formula F, for any of the uses described herein by administering to a patient, such as a human, the enantiomerically enriched compound in an effective amount to achieve the desired effect:

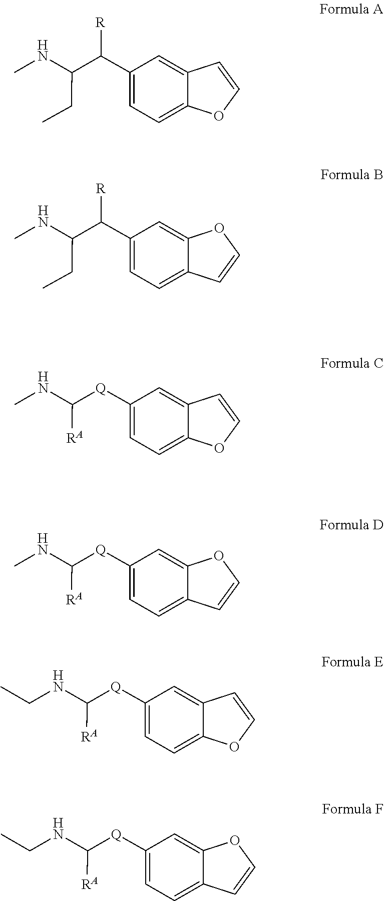

wherein
R is hydrogen or hydroxyl.
R^{A} is —CH₃, —CH₂Y, —CHY₂, —CY₃, —CH₂CH₃, —CH₂CH₂Y, —CH₂CHY₂, —CH₂CY₃, —CH₂OH, or —CH₂CH₂OH;
Q is selected from:

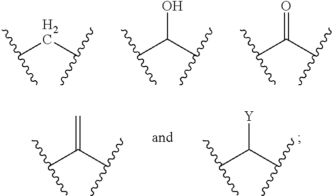

and
Y is halogen.

In certain aspects of these embodiments, one or more selected salt morphic form, morphic salt mixture, or specified salt mixture of compounds of Formulas I-XIII or Formulas A-F can be improved or “tuned” by administering an effective amount to a host such as a human, in need thereof, in a composition of a substantially pure enantiomer (or diastereomer, where relevant), or alternatively, an enantiomerically enriched composition that has an abundance of one enantiomer over the other. In this way, as described above, the enantiomeric forms act differently from each other on various 5-HT receptors, dopamine receptors, nicotinic acetylcholine receptors, and norepinephrine receptors, producing variable effects, and that those effects can be selected for based on desired outcome for the patient.

In certain embodiments, any of the selected salt morphic forms or a salt mixture of compounds or mixtures of the present invention is administered to a patient in an effective amount in conjunction with psychotherapy, cognitive enhancement, or life coaching (pharmacotherapy), or as part of routine medical therapy.

In certain embodiments, compounds of Formula A and Formula B are halogenated, for example by having one or more halogens in place of one or more hydrogens on the ethyl group attached at the alpha carbon.

The present invention also provides salts and salt mixtures that that in certain embodiments can be in methods for the modulation of CNS activity and/or a method for treatment of CNS disorders, including, but not limited to post-traumatic stress and adjustment disorders, comprising administering a salt morphic form, morphic salt mixture, or specified salt mixture described herein of a compound of Formula C or Formula D:

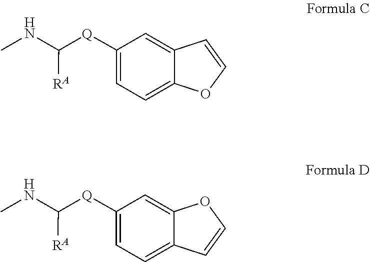

wherein R^{A} is —CH₃, —CH₂Y, —CHY₂, —CY₃, —CH₂CH₃, —CH₂CH₂Y, —CH₂CHY₂, —CH₂CY₃, —CH₂OH, or —CH₂CH₂OH;
Q is selected from:

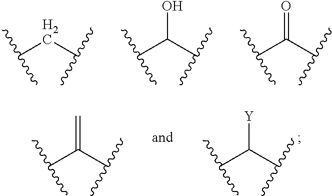

and
Y is halogen.

In one embodiment, compounds of Formula C and Formula D are halogenated, for example by having one or more halogens in place of one or more hydrogens on the alkyl group attached at the alpha carbon, e.g., as defined at position R^{A} (e.g., halogenated alpha-ethyl or alpha-methyl compounds).

The present invention also provides salts and salt mixtures that that in certain embodiments can be in methods for the modulation of CNS activity and/or a method for treatment of CNS disorders, including, but not limited to post-traumatic stress and adjustment disorders, comprising administering a salt morphic form, morphic salt mixture, or specified salt mixture described herein of a compound of Formula E or Formula F:

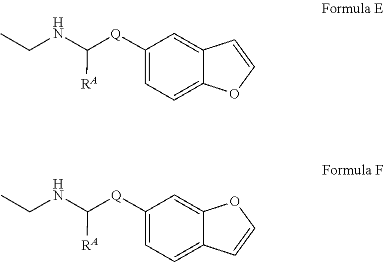

wherein R^{A} is —CH₃, —CH₂Y, —CHY₂, —CY₃, —CH₂CH₃, —CH₂CH₂Y, —CH₂CHY₂, —CH₂CY₃, —CH₂OH, or —CH₂CH₂OH;
Q is selected from:

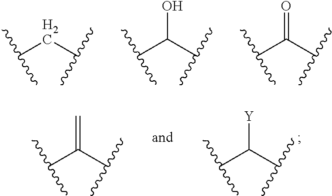

and
Y is halogen.

In one embodiment, compounds of Formula E and Formula F are halogenated, for example by having one or more halogens in place of one or more hydrogens on the alkyl group attached at the alpha carbon, e.g., as defined at position R^{A} (e.g., halogenated alpha-ethyl or alpha-methyl compounds).

In certain embodiments the present invention uses an enantiomerically enriched compounds Bk-5-MAPB and Bk-6-MAPB or a pharmaceutically acceptable salt or mixed salt thereof:

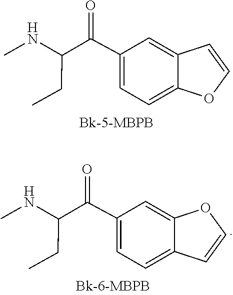 [SMILES: CCC(NC)C(=O)c1ccc2occc2c1 ; CCC(NC)C(=O)c1ccc2ccoc2c1]

The salt morphic form or salt mixture of compounds may be provided in a composition that is enantiomerically enriched, such as a mixture of enantiomers in which one enantiomer is present in excess, in particular to the extent of 60% or more, 70% or more, 75% or more, 80% or more, 90% or more, 95% or more, or 98% or more, including 100%.

In certain embodiments, the salt or mixture of salts of the present invention is of a

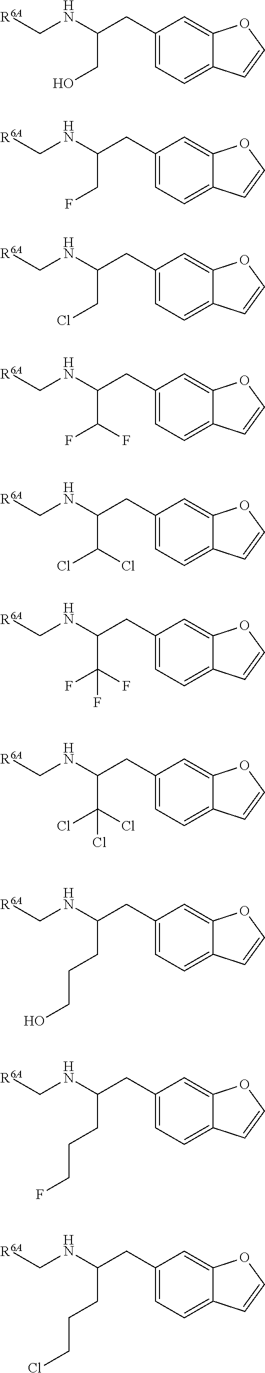

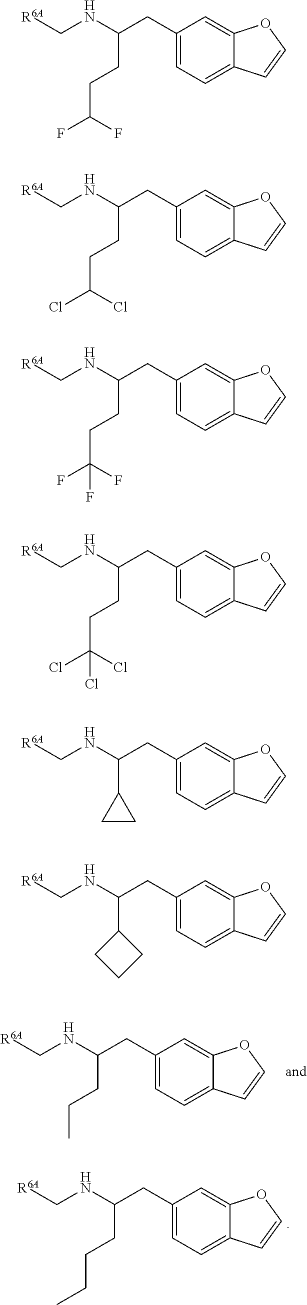

In certain embodiments, the salt or mixture of salts of the present invention is of a

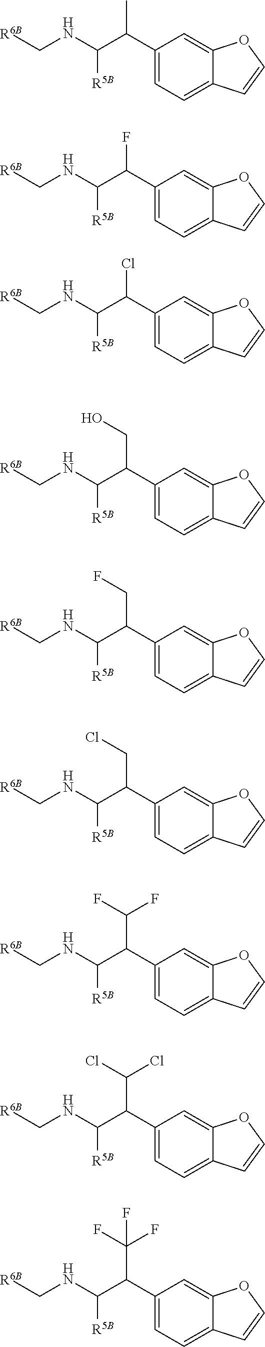

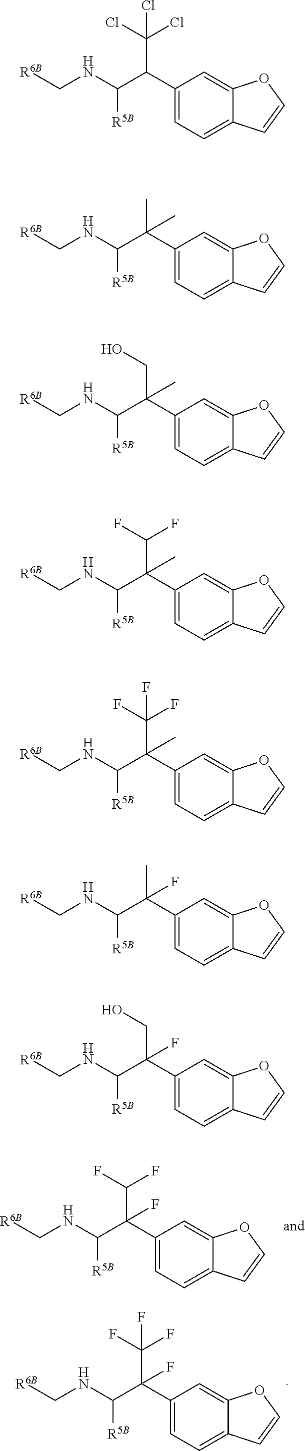

In certain embodiments, the salt or mixture of salts of the present invention is of a

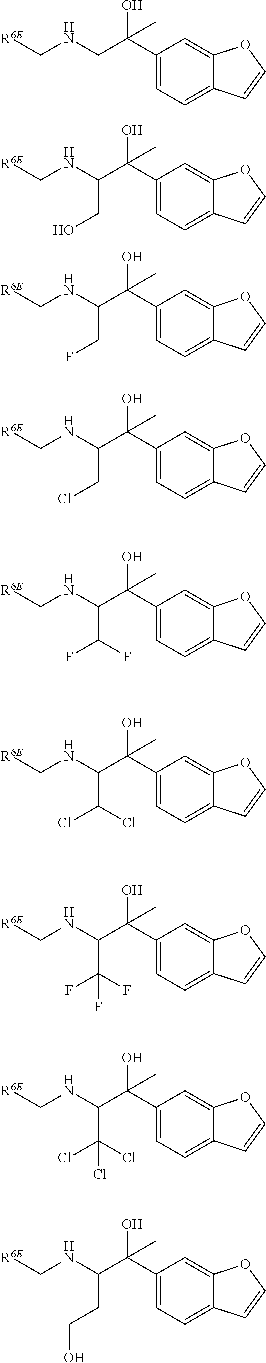

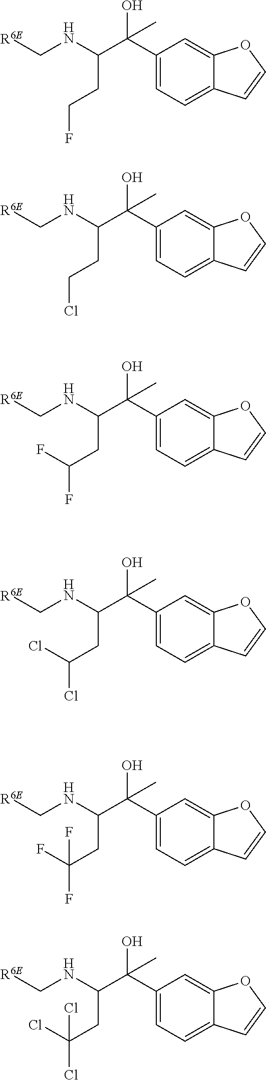

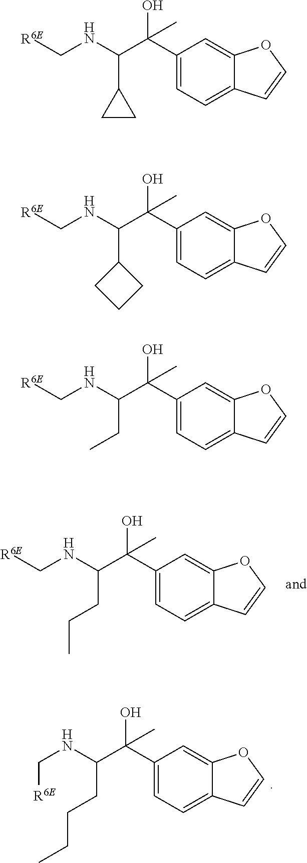

In certain embodiments, the salt or mixture of salts of the present invention is of a

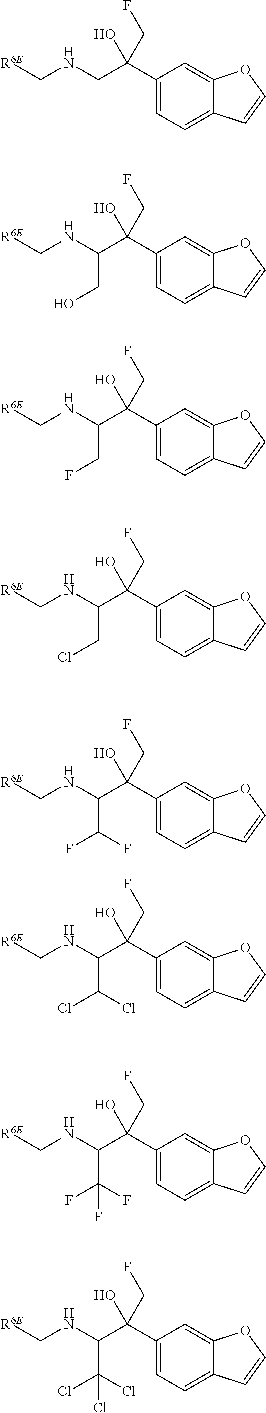

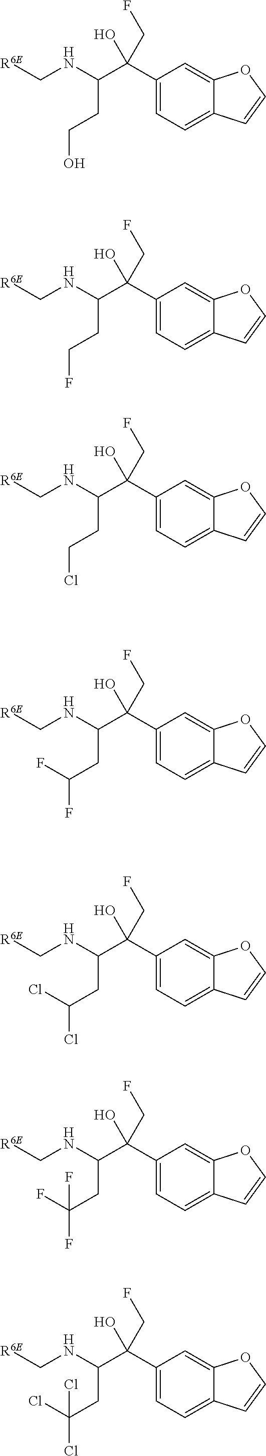

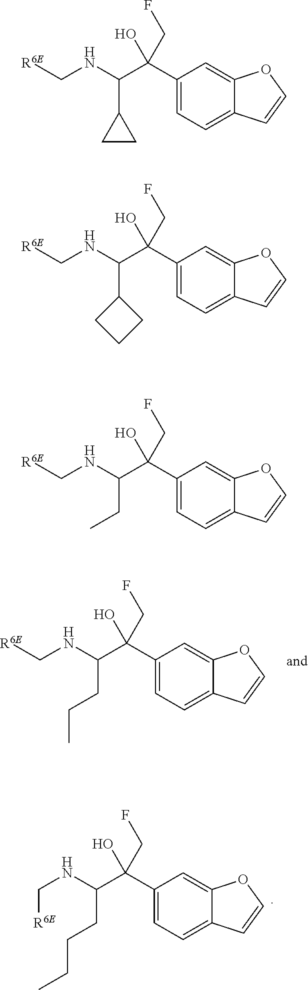

In certain embodiments, the salt or mixture of salts of the present invention is of a

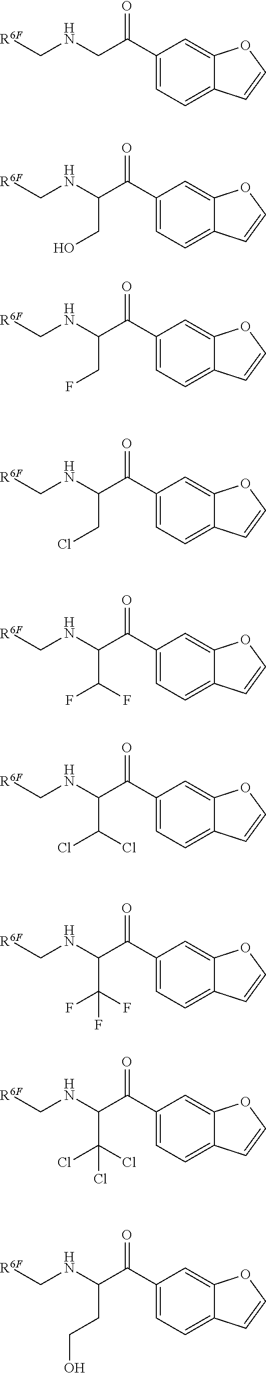

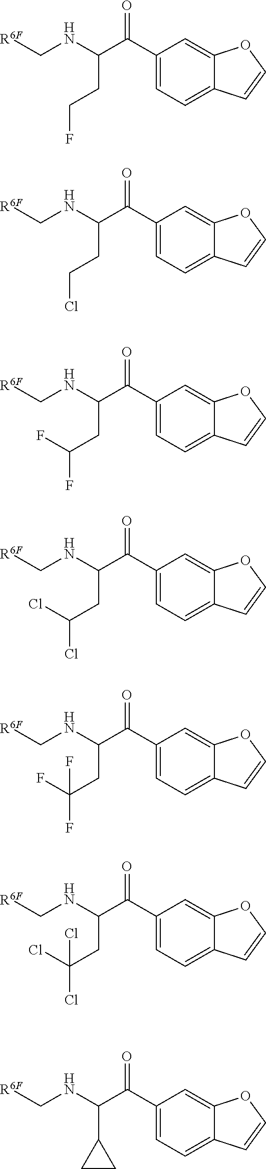

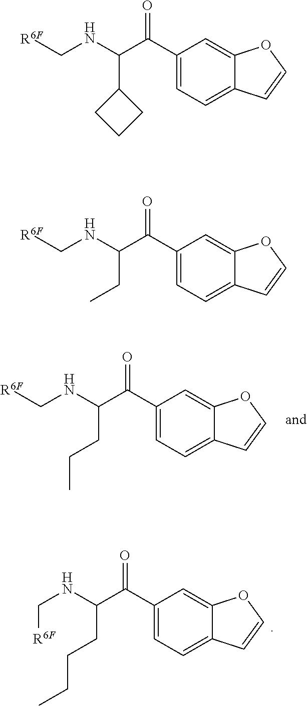

In certain embodiments, the salt or mixture of salts of the present invention is of a

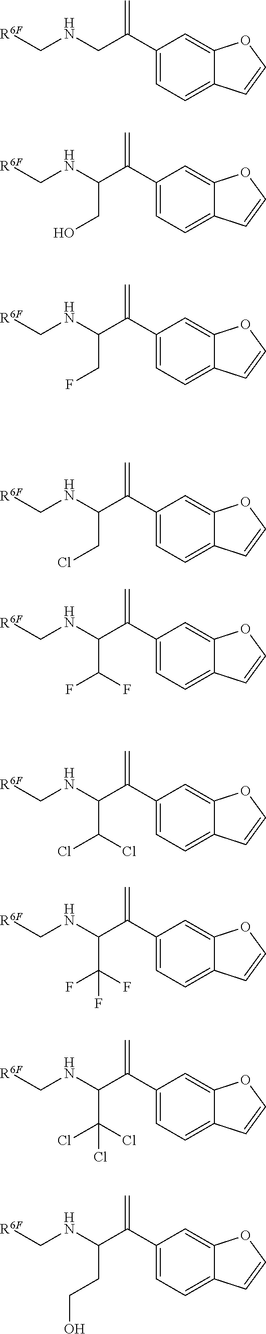

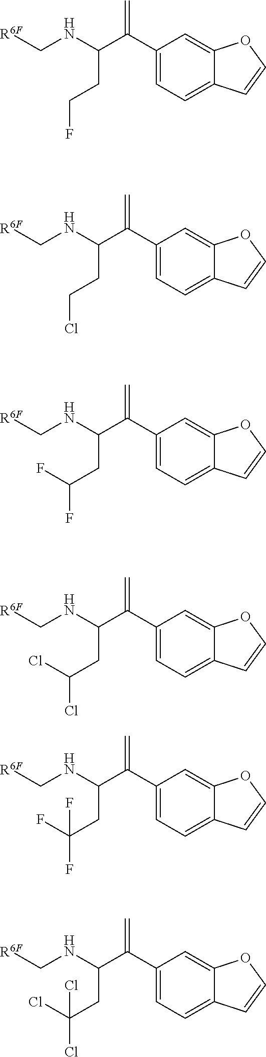

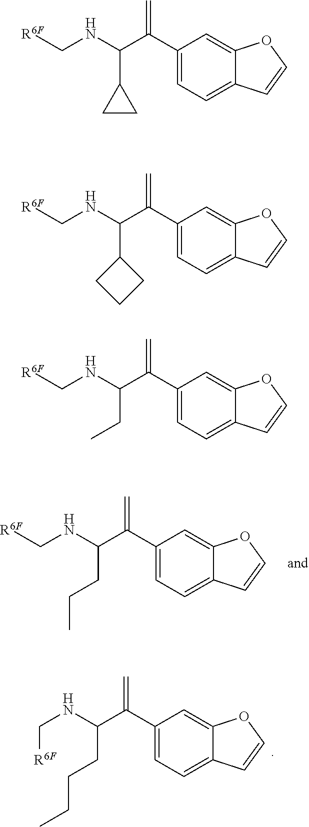

In certain embodiments, the salt or mixture of salts of the present invention is of a

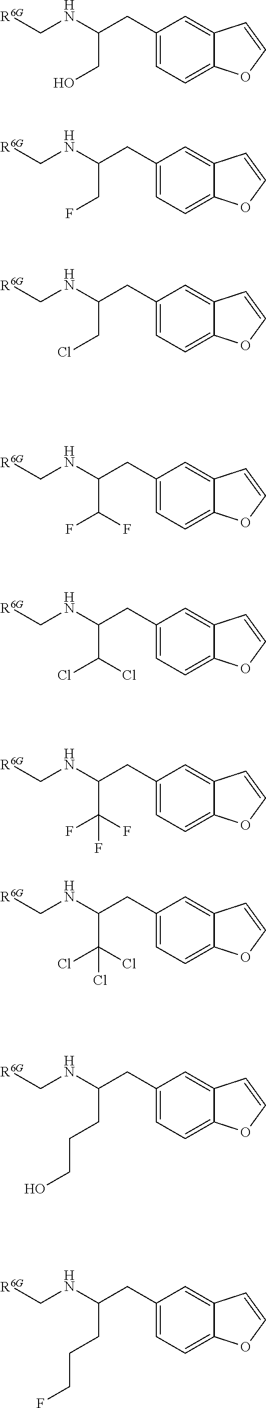

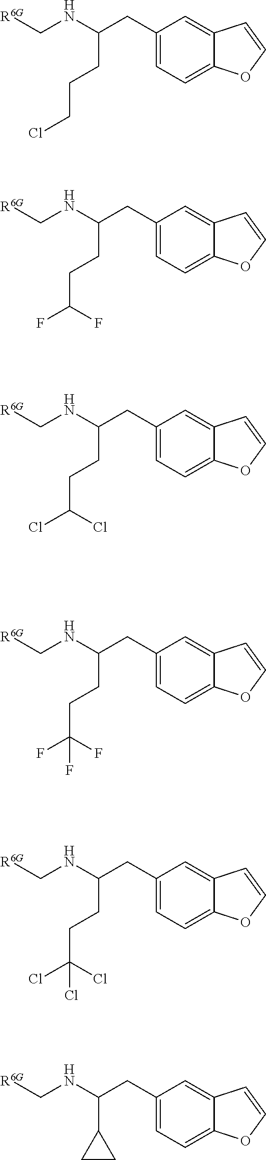

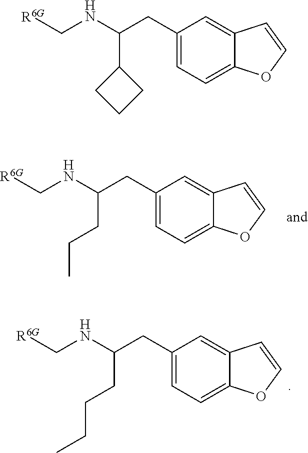

In certain embodiments, the salt or mixture of salts of the present invention is of a compound selected from:

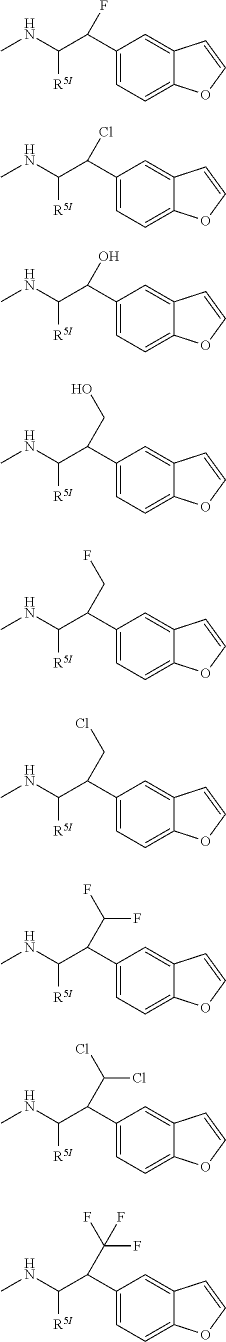

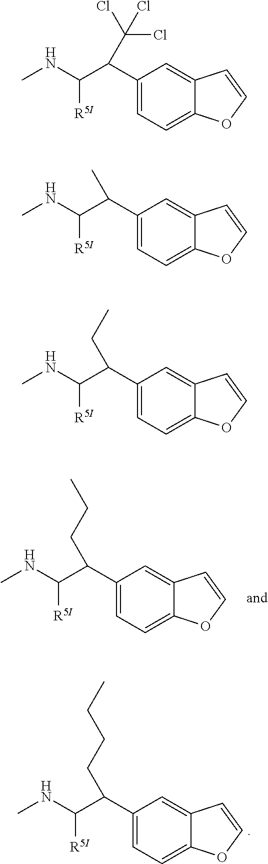

In certain embodiments, the salt or mixture of salts of the present invention is of a

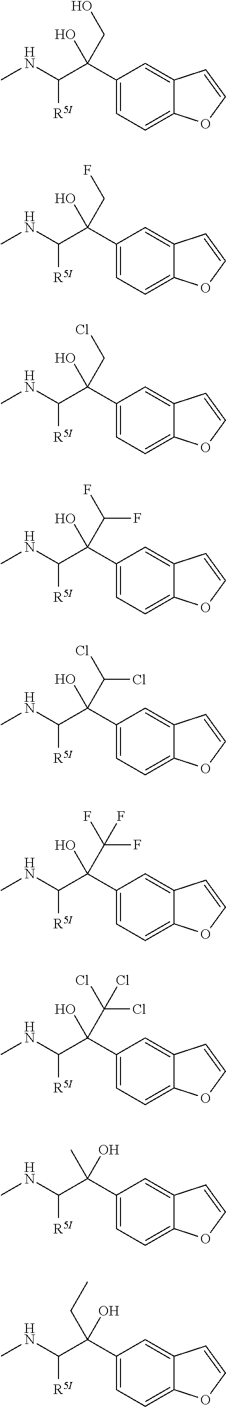

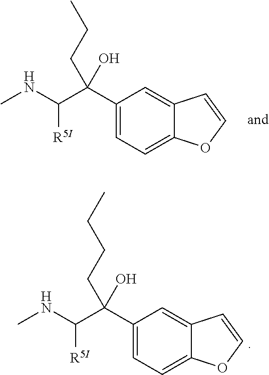

In certain embodiments, the salt or mixture of salts of the present invention is of a

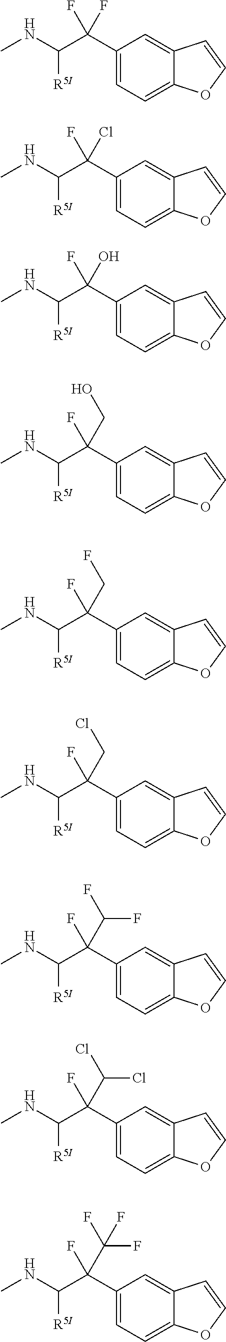

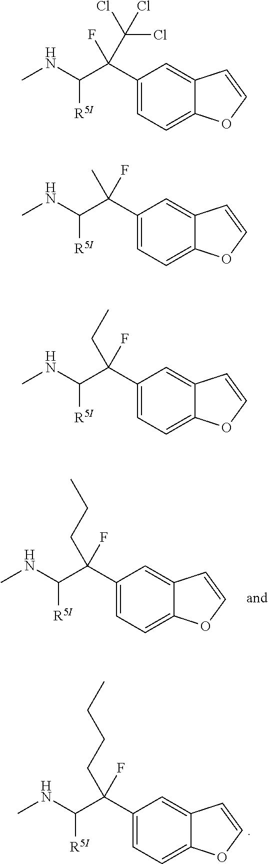

In certain embodiments, the salt or mixture of salts of the present invention is of a

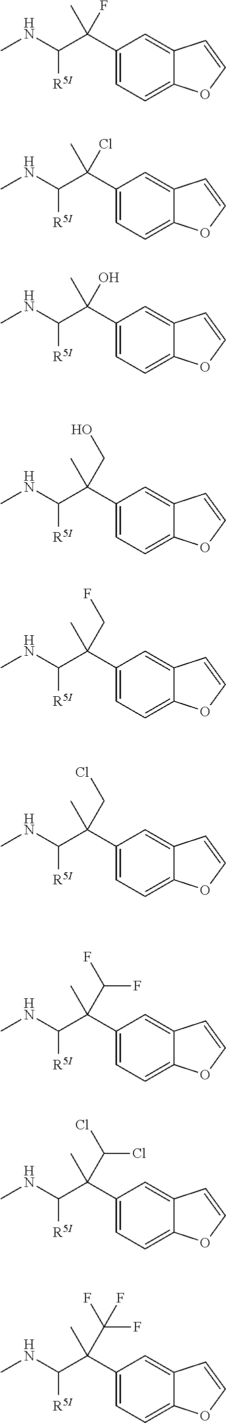

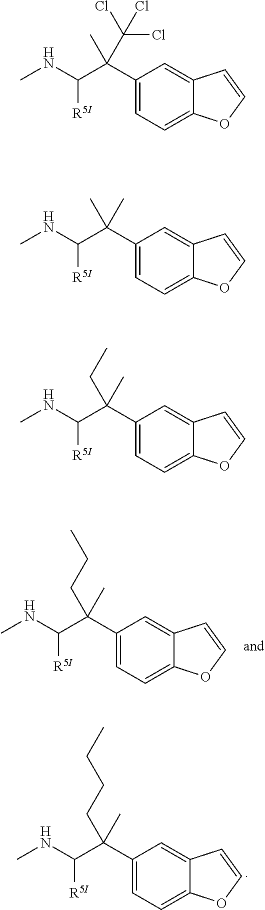

In certain embodiments, the salt or mixture of salts of the present invention is of a

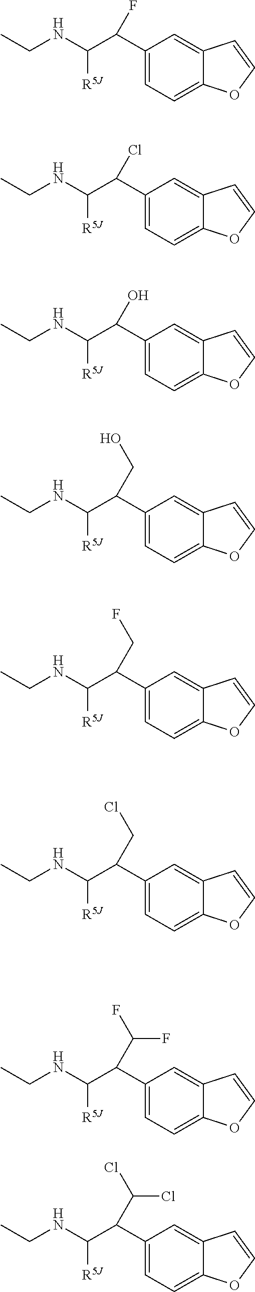

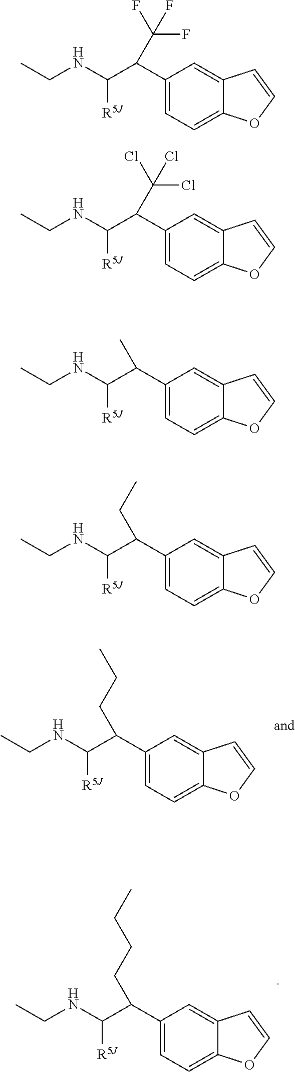

In certain embodiments, the salt or mixture of salts of the present invention is of a

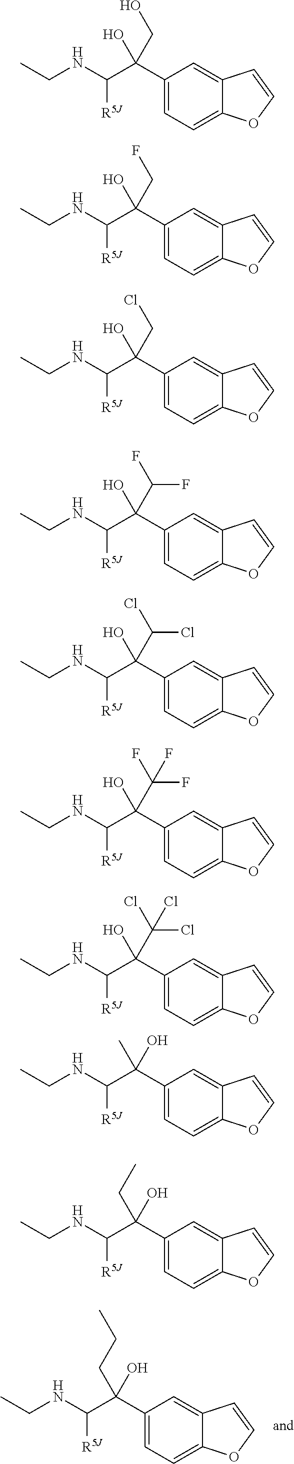

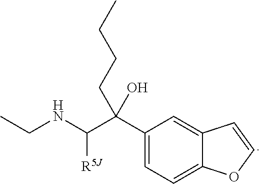

In certain embodiments, the salt or mixture of salts of the present invention is of a compound selected from:

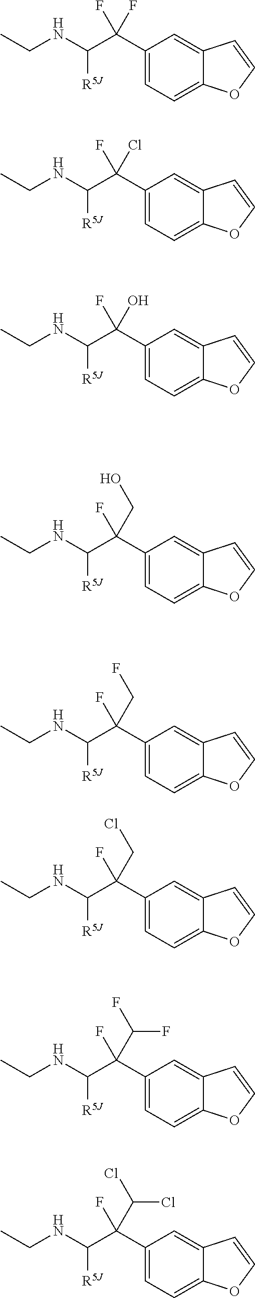

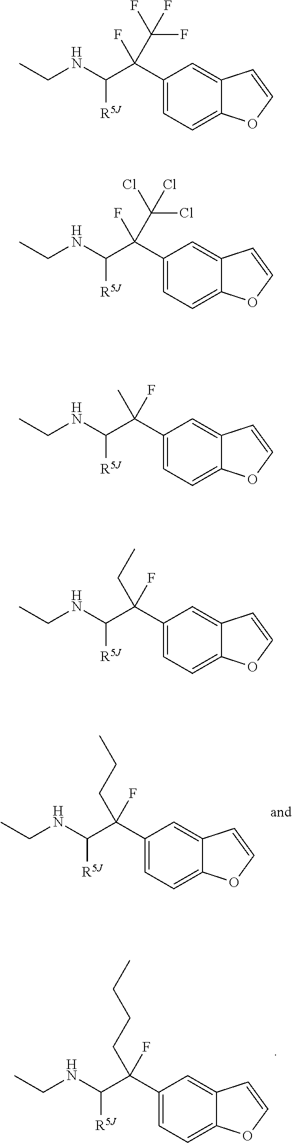

In certain embodiments, the salt or mixture of salts of the present invention is of a compound selected from:

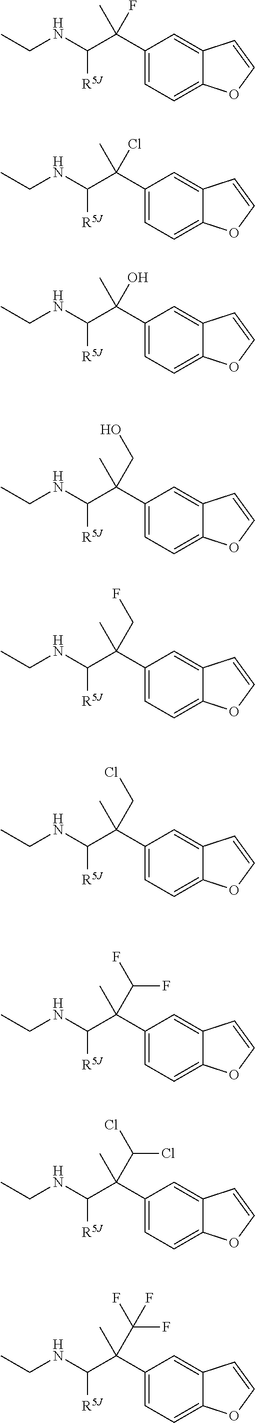

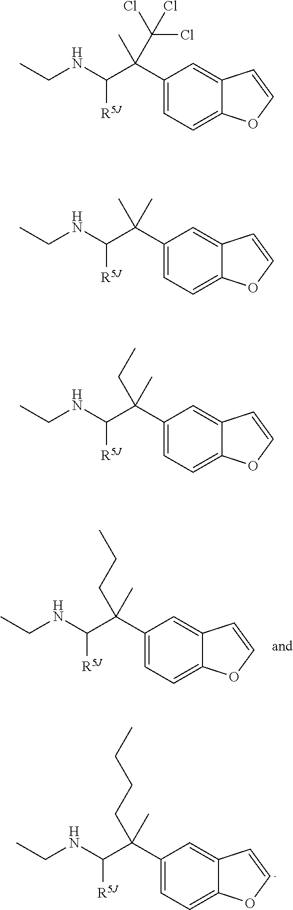

In certain embodiments, the salt or mixture of salts of the present invention is of a

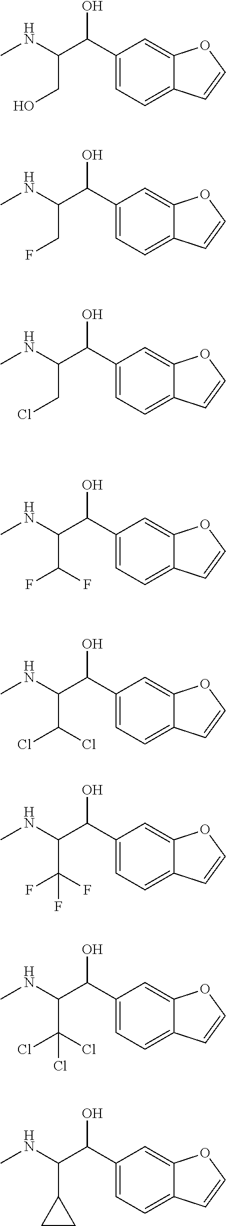

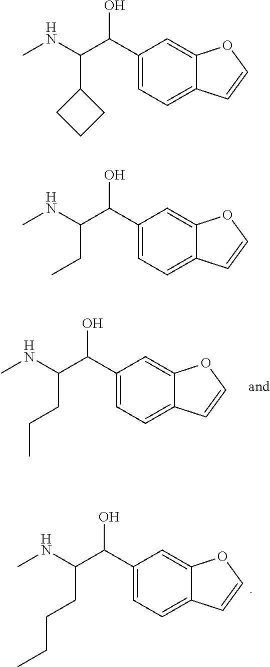 [SMILES: CNC(C1CCC1)C(O)c1ccc2ccoc2c1 ; CCC(NC)C(O)c1ccc2ccoc2c1 ; CCCC(NC)C(O)c1ccc2ccoc2c1 ; CCCCC(NC)C(O)c1ccc2ccoc2c1]

In certain embodiments, the salt or mixture of salts of the present invention is of a compound selected from:

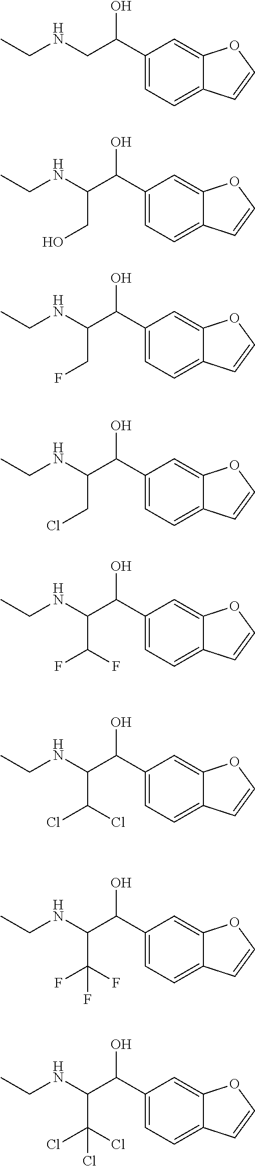

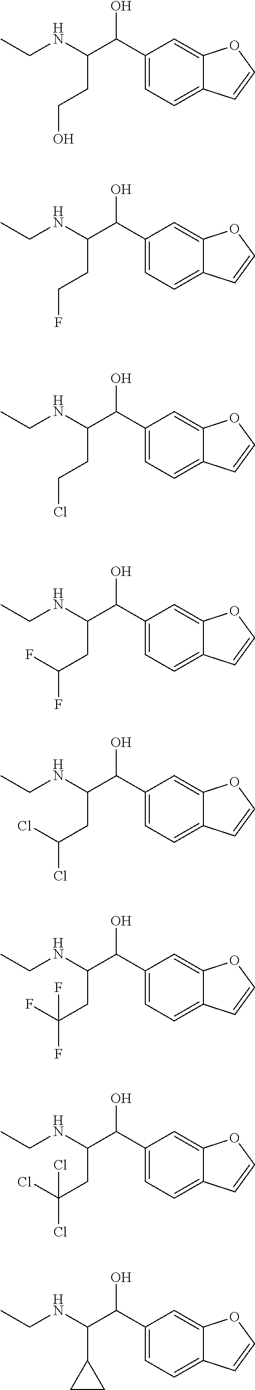

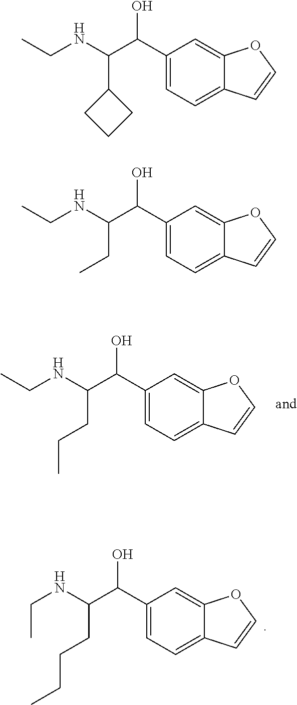

In certain embodiments, the salt or mixture of salts of the present invention is of a

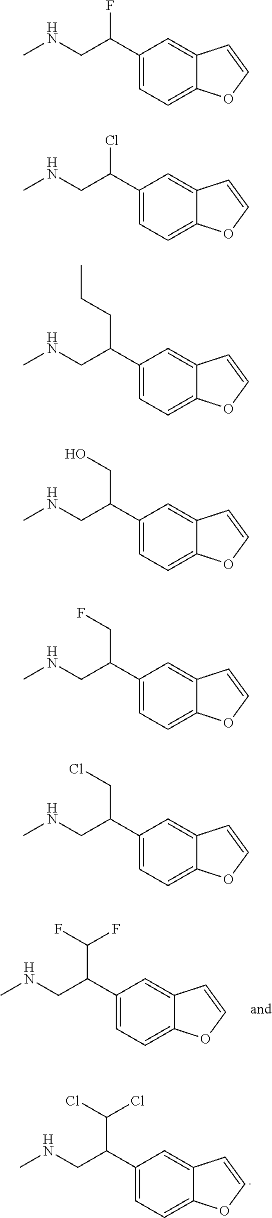

In certain embodiments, the salt or mixture of salts of the present invention is of a

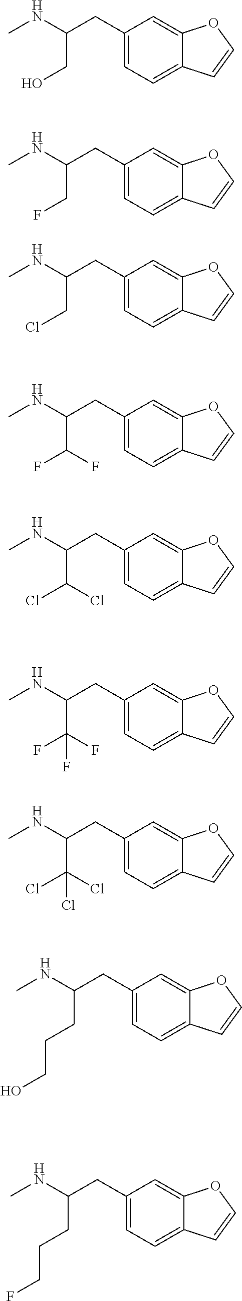

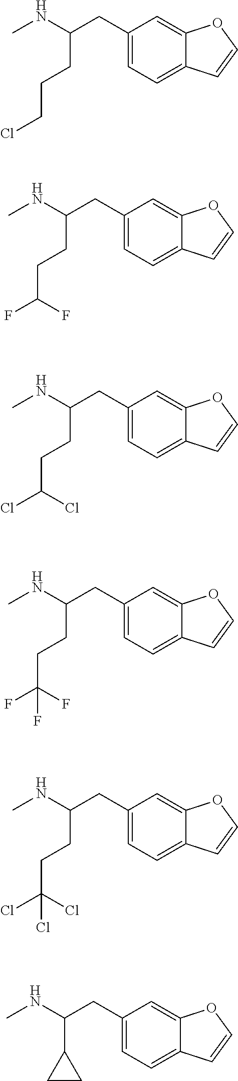 [SMILES: CNC(CCCCl)Cc1ccc2ccoc2c1 ; CNC(CCC(F)F)Cc1ccc2ccoc2c1 ; CNC(CCC(Cl)Cl)Cc1ccc2ccoc2c1 ; CNC(CCC(F)(F)F)Cc1ccc2ccoc2c1 ; CNC(CCC(Cl)(Cl)Cl)Cc1ccc2ccoc2c1 ; CNC(Cc1ccc2ccoc2c1)C1CC1]

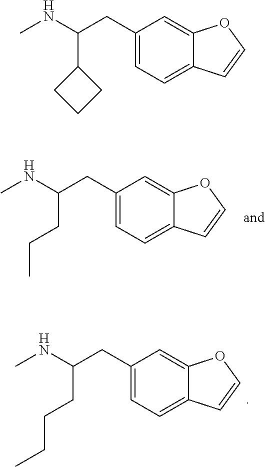 [SMILES: CNC(Cc1ccc2ccoc2c1)C1CCC1 ; CCCC(Cc1ccc2ccoc2c1)NC ; CCCCC(Cc1ccc2ccoc2c1)NC]

In certain embodiments, the salt or mixture of salts of the present invention is of a compound selected from:

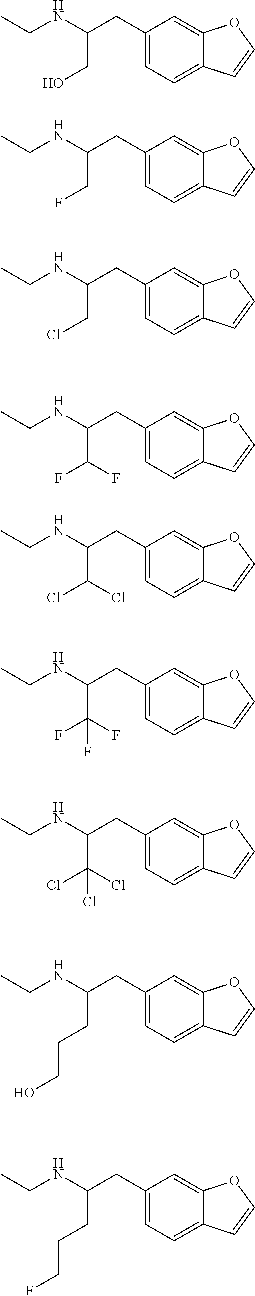

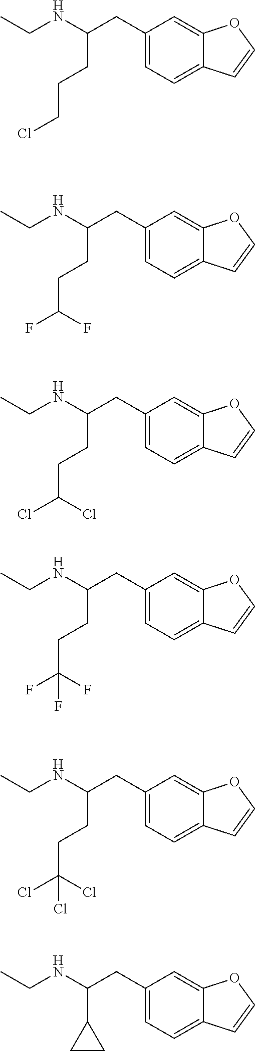 [SMILES: CCNC(CCCCl)Cc1ccc2ccoc2c1 ; CCNC(CCC(F)F)Cc1ccc2ccoc2c1 ; CCNC(CCC(Cl)Cl)Cc1ccc2ccoc2c1 ; CCNC(CCC(F)(F)F)Cc1ccc2ccoc2c1 ; CCNC(CCC(Cl)(Cl)Cl)Cc1ccc2ccoc2c1 ; CCNC(Cc1ccc2ccoc2c1)C1CC1]

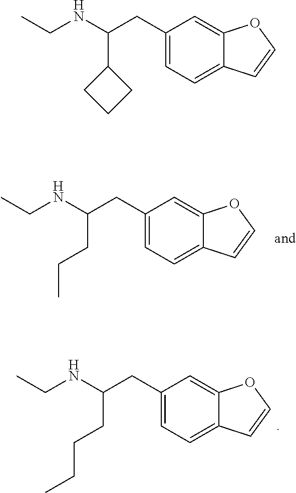 [SMILES: CCNC(Cc1ccc2ccoc2c1)C1CCC1 ; CCCC(Cc1ccc2ccoc2c1)NCC ; CCCCC(Cc1ccc2ccoc2c1)NCC]

In certain embodiments, the salt or mixture of salts of the present invention is of a

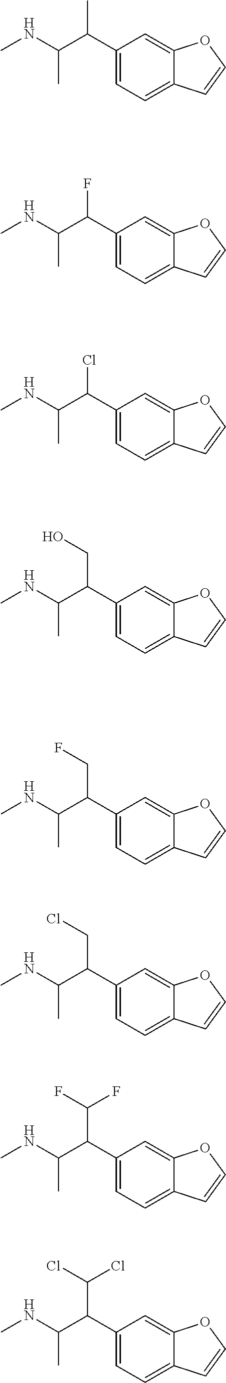

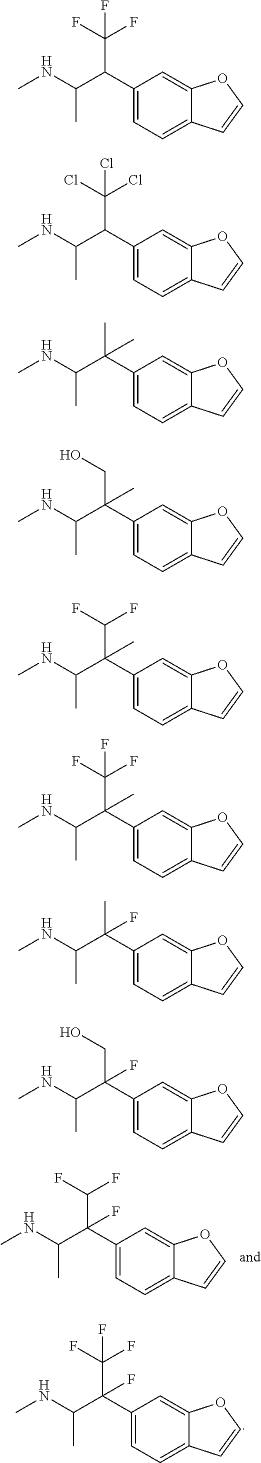

In certain embodiments, the salt or mixture of salts of the present invention is of a

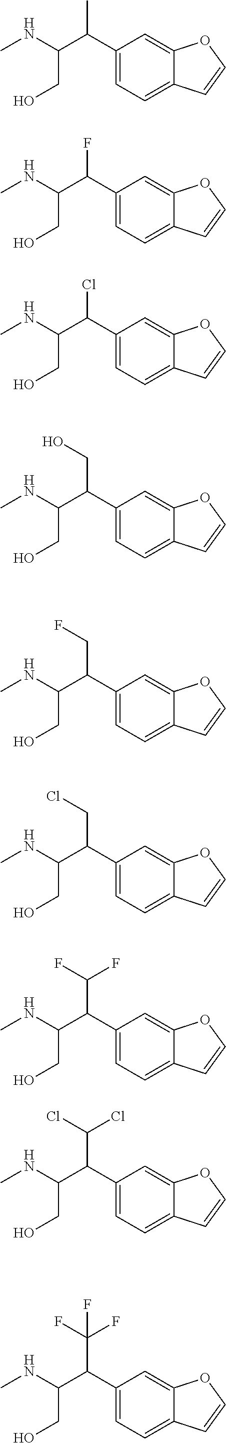

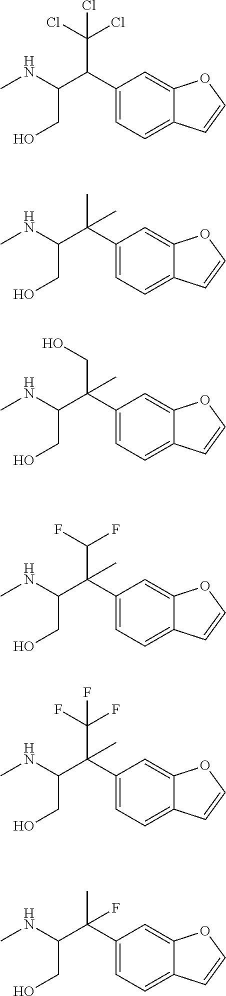 [SMILES: CNC(CO)C(c1ccc2ccoc2c1)C(Cl)(Cl)Cl ; CNC(CO)C(C)(C)c1ccc2ccoc2c1 ; CNC(CO)C(C)(CO)c1ccc2ccoc2c1 ; CNC(CO)C(C)(c1ccc2ccoc2c1)C(F)F ; CNC(CO)C(C)(c1ccc2ccoc2c1)C(F)(F)F ; CNC(CO)C(C)(F)c1ccc2ccoc2c1]

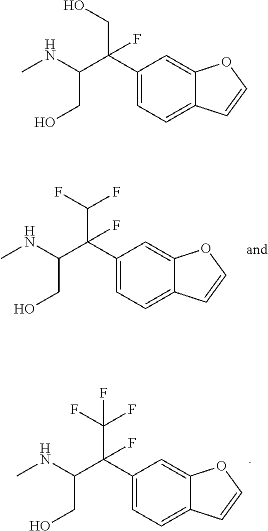 [SMILES: CNC(CO)C(F)(CO)c1ccc2ccoc2c1 ; CNC(CO)C(F)(c1ccc2ccoc2c1)C(F)F ; CNC(CO)C(F)(c1ccc2ccoc2c1)C(F)(F)F]

In certain embodiments, the salt or mixture of salts of the present invention is of a

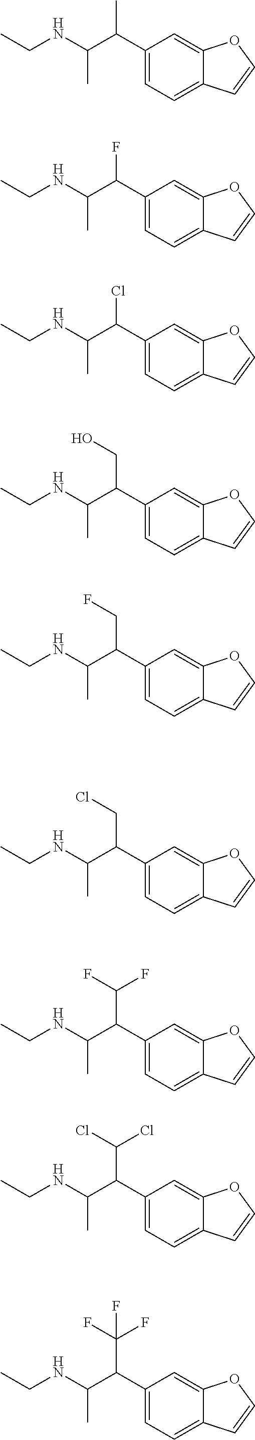

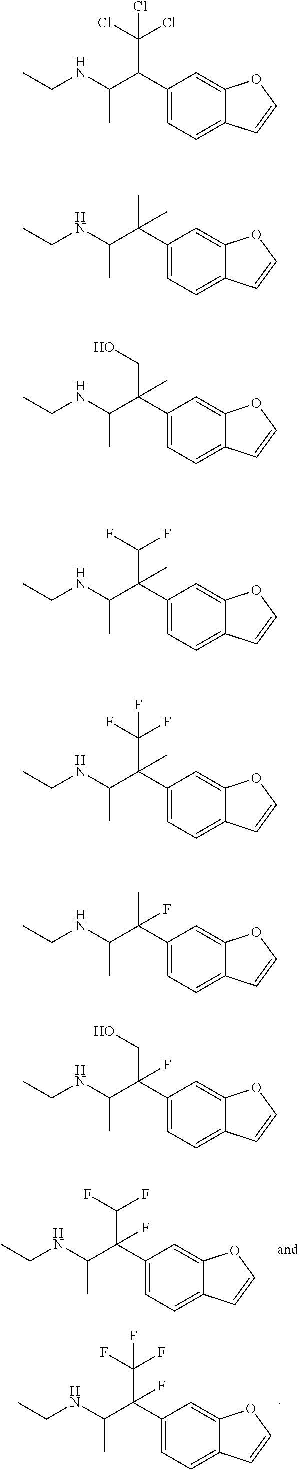

In certain embodiments, the salt or mixture of salts of the present invention is of a

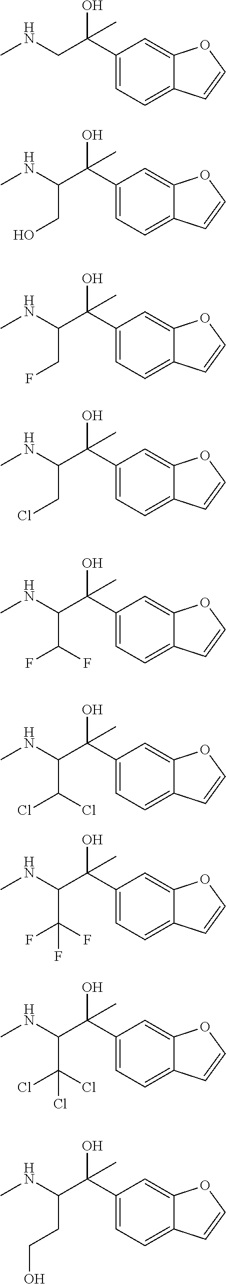

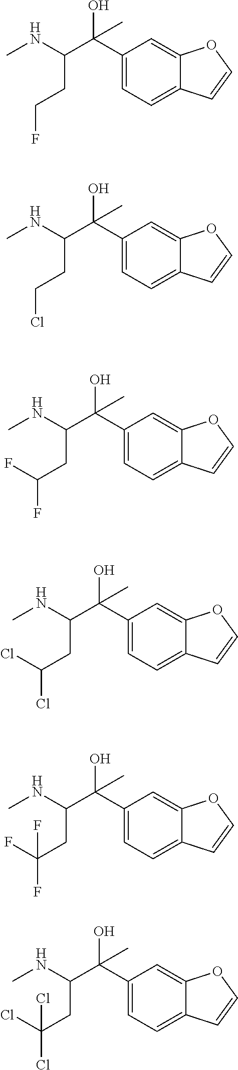 [SMILES: CNC(CCF)C(C)(O)c1ccc2ccoc2c1 ; CNC(CCCl)C(C)(O)c1ccc2ccoc2c1 ; CNC(CC(F)F)C(C)(O)c1ccc2ccoc2c1 ; CNC(CC(Cl)Cl)C(C)(O)c1ccc2ccoc2c1 ; CNC(CC(F)(F)F)C(C)(O)c1ccc2ccoc2c1 ; CNC(CC(Cl)(Cl)Cl)C(C)(O)c1ccc2ccoc2c1]

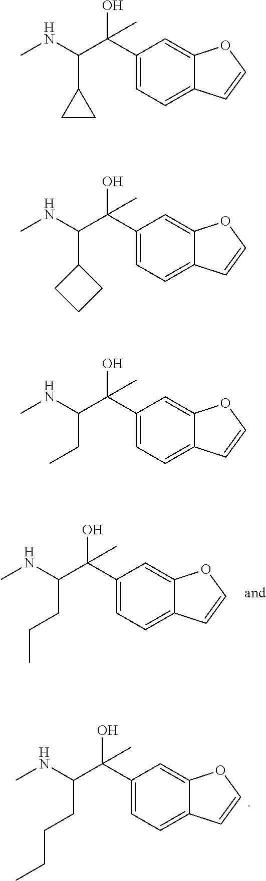 [SMILES: CNC(C1CC1)C(C)(O)c1ccc2ccoc2c1 ; CNC(C1CCC1)C(C)(O)c1ccc2ccoc2c1 ; CCC(NC)C(C)(O)c1ccc2ccoc2c1 ; CCCC(NC)C(C)(O)c1ccc2ccoc2c1 ; CCCCC(NC)C(C)(O)c1ccc2ccoc2c1]

In certain embodiments, the salt or mixture of salts of the present invention is of a

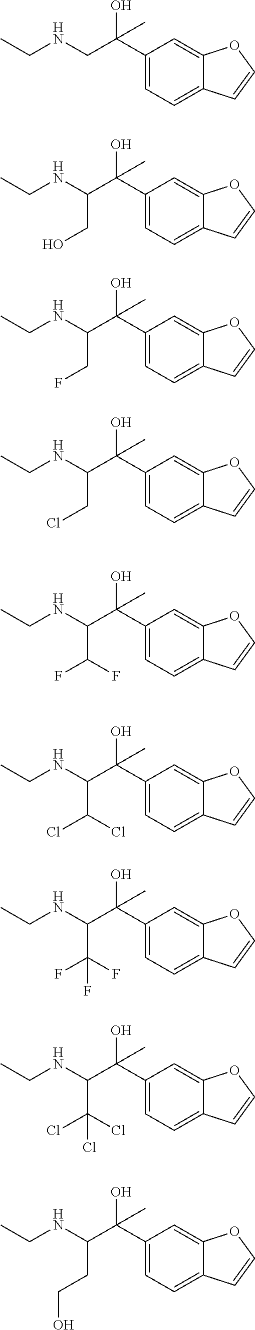

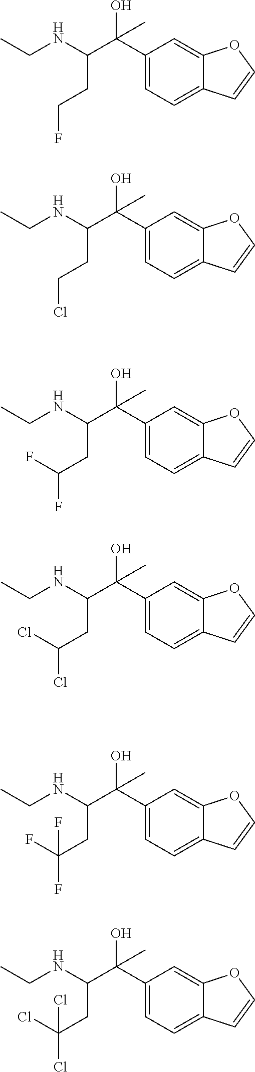 [SMILES: CCNC(CCF)C(C)(O)c1ccc2ccoc2c1 ; CCNC(CCCl)C(C)(O)c1ccc2ccoc2c1 ; CCNC(CC(F)F)C(C)(O)c1ccc2ccoc2c1 ; CCNC(CC(Cl)Cl)C(C)(O)c1ccc2ccoc2c1 ; CCNC(CC(F)(F)F)C(C)(O)c1ccc2ccoc2c1 ; CCNC(CC(Cl)(Cl)Cl)C(C)(O)c1ccc2ccoc2c1]

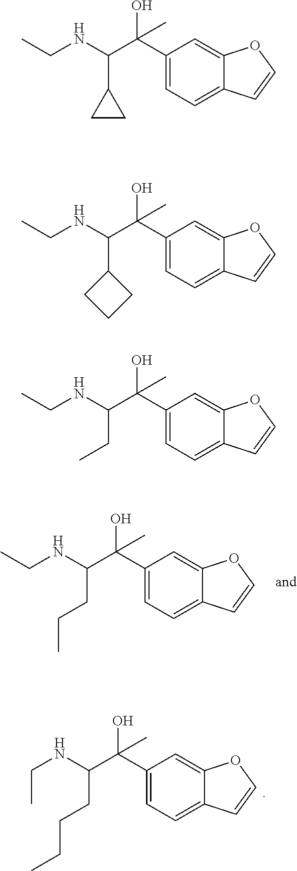 [SMILES: CCNC(C1CC1)C(C)(O)c1ccc2ccoc2c1 ; CCNC(C1CCC1)C(C)(O)c1ccc2ccoc2c1 ; CCNC(CC)C(C)(O)c1ccc2ccoc2c1 ; CCCC(NCC)C(C)(O)c1ccc2ccoc2c1 ; CCCCC(NCC)C(C)(O)c1ccc2ccoc2c1]

In certain embodiments, the salt or mixture of salts of the present invention is of a

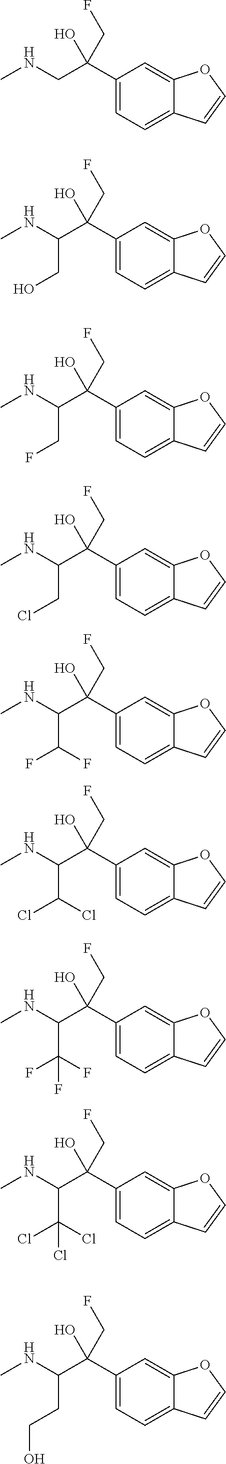

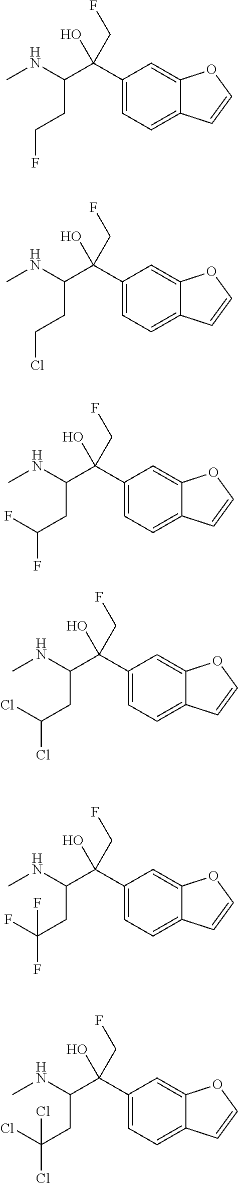 [SMILES: CNC(CCF)C(O)(CF)c1ccc2ccoc2c1 ; CNC(CCCl)C(O)(CF)c1ccc2ccoc2c1 ; CNC(CC(F)F)C(O)(CF)c1ccc2ccoc2c1 ; CNC(CC(Cl)Cl)C(O)(CF)c1ccc2ccoc2c1 ; CNC(CC(F)(F)F)C(O)(CF)c1ccc2ccoc2c1 ; CNC(CC(Cl)(Cl)Cl)C(O)(CF)c1ccc2ccoc2c1]

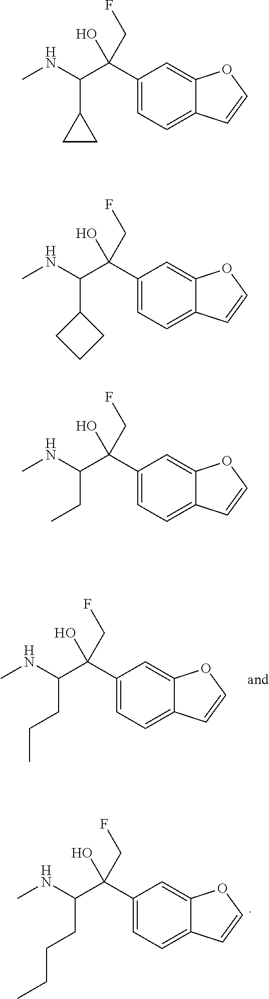 [SMILES: CNC(C1CC1)C(O)(CF)c1ccc2ccoc2c1 ; CNC(C1CCC1)C(O)(CF)c1ccc2ccoc2c1 ; CCC(NC)C(O)(CF)c1ccc2ccoc2c1 ; CCCC(NC)C(O)(CF)c1ccc2ccoc2c1 ; CCCCC(NC)C(O)(CF)c1ccc2ccoc2c1]

In certain embodiments, the salt or mixture of salts of the present invention is of a

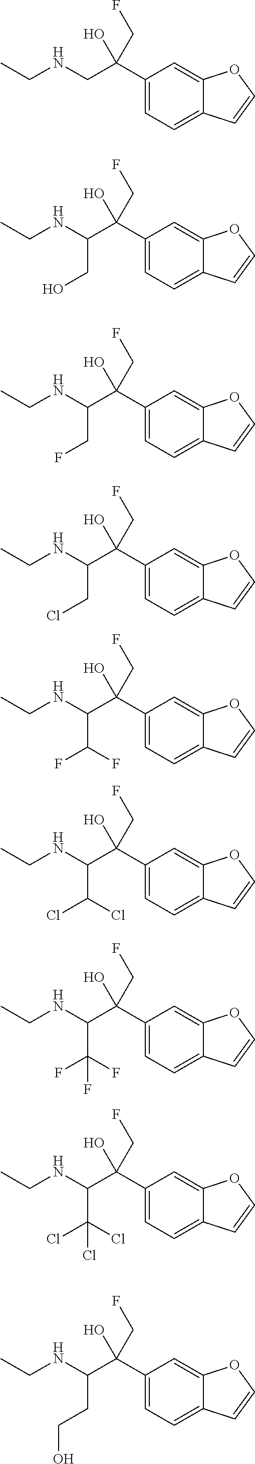

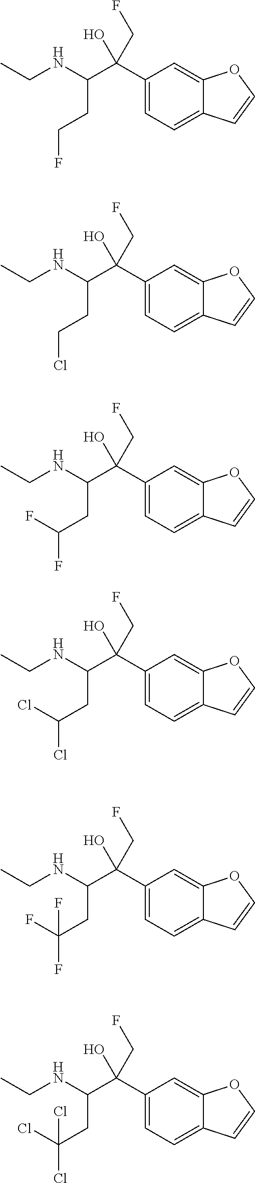 [SMILES: CCNC(CCF)C(O)(CF)c1ccc2ccoc2c1 ; CCNC(CCCl)C(O)(CF)c1ccc2ccoc2c1 ; CCNC(CC(F)F)C(O)(CF)c1ccc2ccoc2c1 ; CCNC(CC(Cl)Cl)C(O)(CF)c1ccc2ccoc2c1 ; CCNC(CC(F)(F)F)C(O)(CF)c1ccc2ccoc2c1 ; CCNC(CC(Cl)(Cl)Cl)C(O)(CF)c1ccc2ccoc2c1]

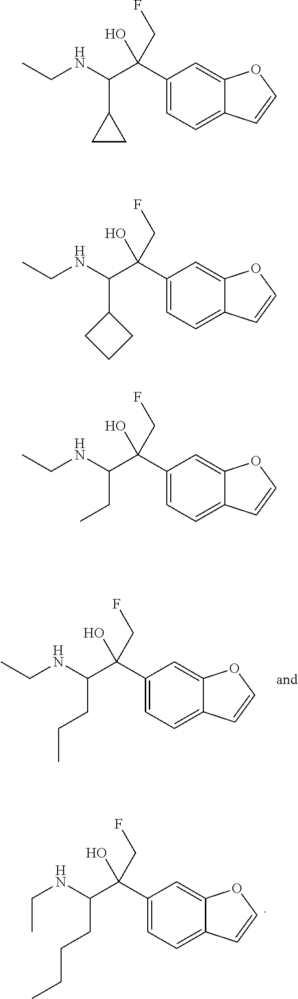 [SMILES: CCNC(C1CC1)C(O)(CF)c1ccc2ccoc2c1 ; CCNC(C1CCC1)C(O)(CF)c1ccc2ccoc2c1 ; CCNC(CC)C(O)(CF)c1ccc2ccoc2c1 ; CCCC(NCC)C(O)(CF)c1ccc2ccoc2c1 ; CCCCC(NCC)C(O)(CF)c1ccc2ccoc2c1]

In certain embodiments, the salt or mixture of salts of the present invention is of a

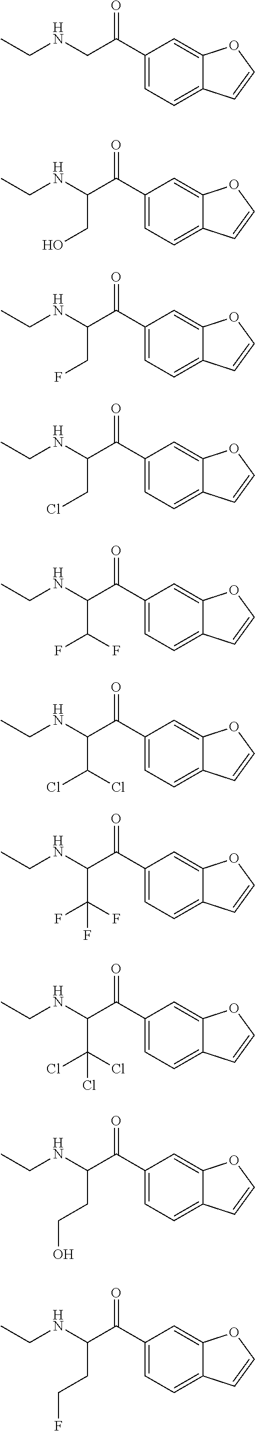

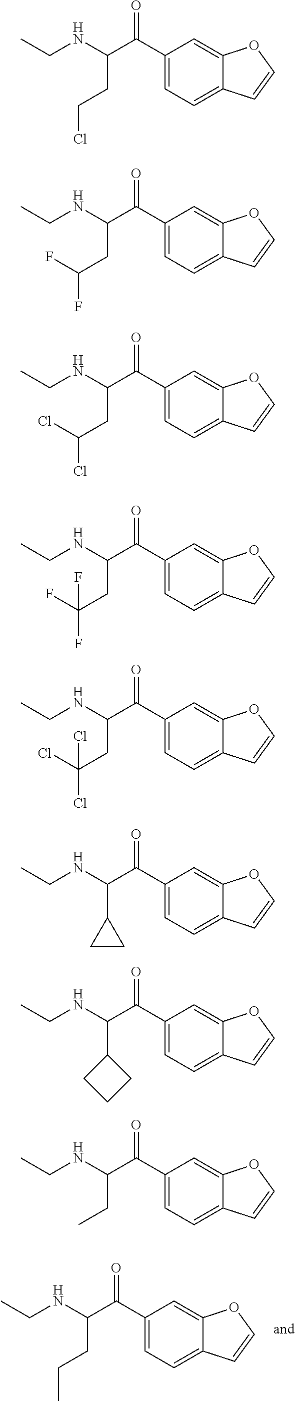

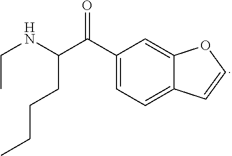 [SMILES: CCCCC(NCC)C(=O)c1ccc2ccoc2c1]

In certain embodiments, the salt or mixture of salts of the present invention is of a

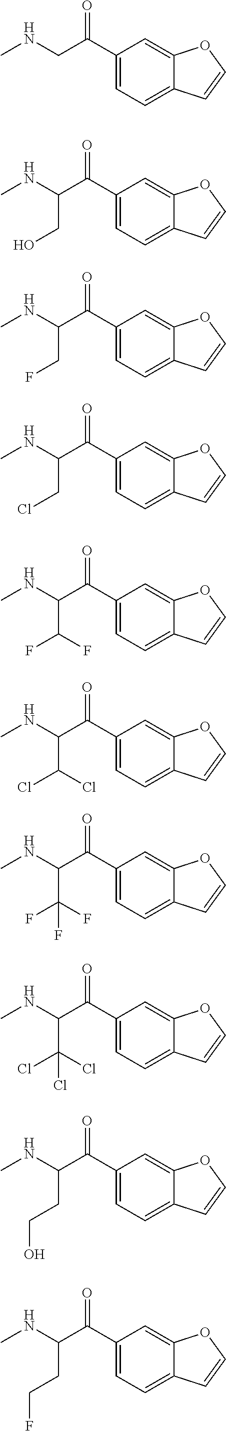

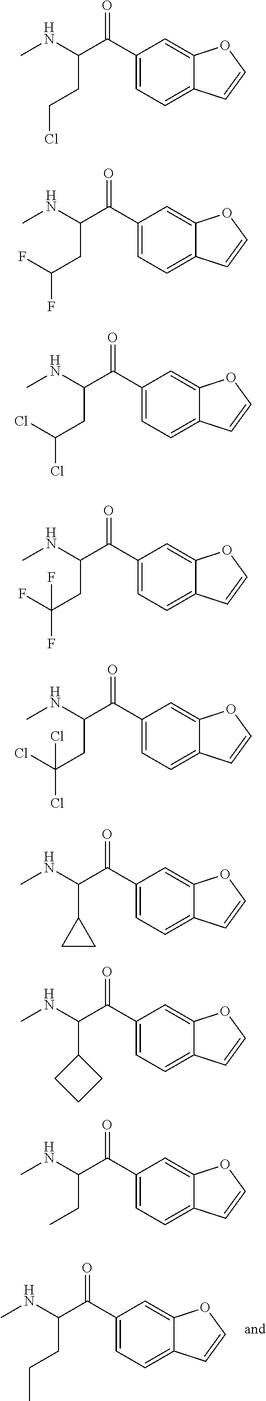

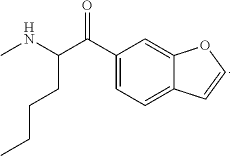 [SMILES: CCCCC(NC)C(=O)c1ccc2ccoc2c1]

In certain embodiments, the salt or mixture of salts of the present invention is of a

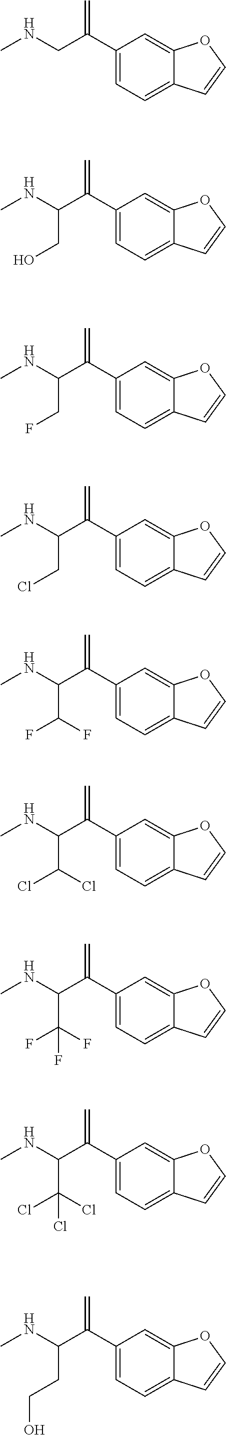

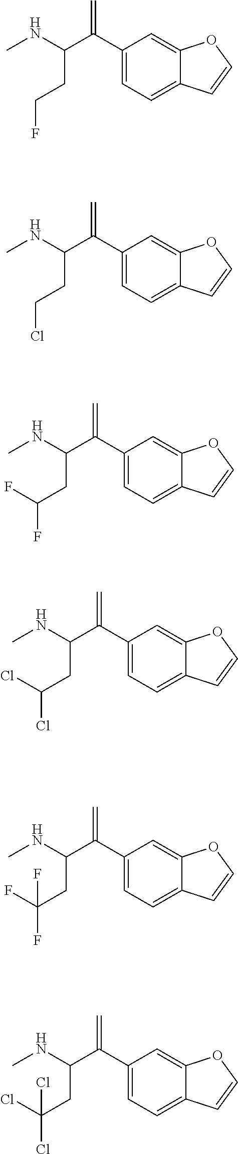 [SMILES: C=C(c1ccc2ccoc2c1)C(CCF)NC ; C=C(c1ccc2ccoc2c1)C(CCCl)NC ; C=C(c1ccc2ccoc2c1)C(CC(F)F)NC ; C=C(c1ccc2ccoc2c1)C(CC(Cl)Cl)NC ; C=C(c1ccc2ccoc2c1)C(CC(F)(F)F)NC ; C=C(c1ccc2ccoc2c1)C(CC(Cl)(Cl)Cl)NC]

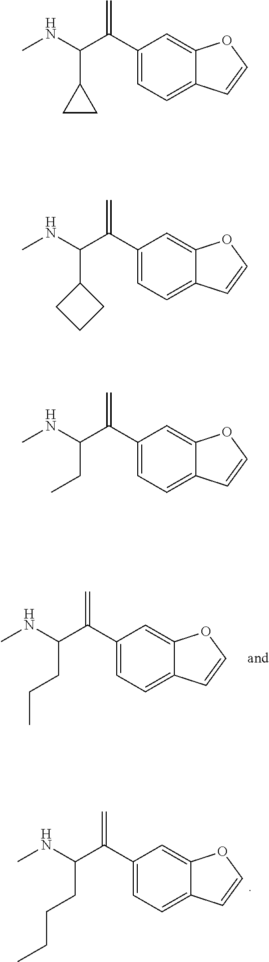 [SMILES: C=C(c1ccc2ccoc2c1)C(NC)C1CC1 ; C=C(c1ccc2ccoc2c1)C(NC)C1CCC1 ; C=C(c1ccc2ccoc2c1)C(CC)NC ; C=C(c1ccc2ccoc2c1)C(CCC)NC ; C=C(c1ccc2ccoc2c1)C(CCCC)NC]

In certain embodiments, the salt or mixture of salts of the present invention is of a compound selected from:

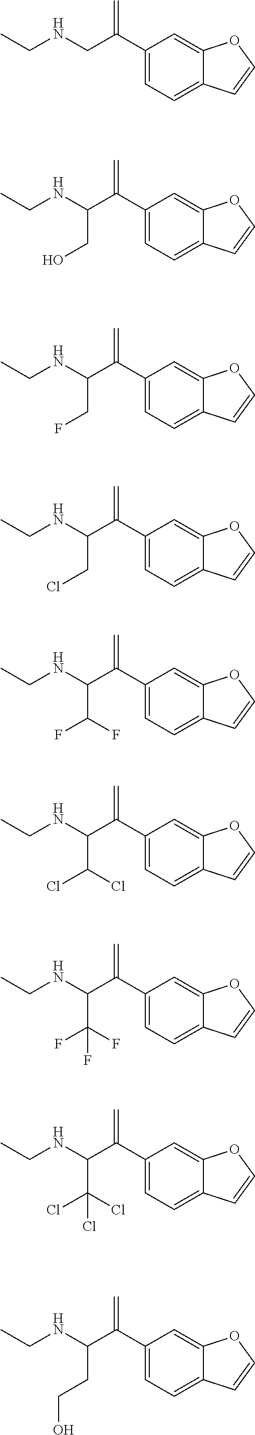

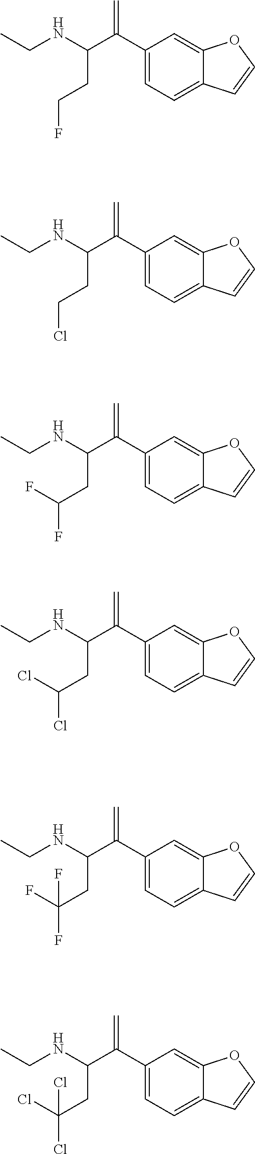 [SMILES: C=C(c1ccc2ccoc2c1)C(CCF)NCC ; C=C(c1ccc2ccoc2c1)C(CCCl)NCC ; C=C(c1ccc2ccoc2c1)C(CC(F)F)NCC ; C=C(c1ccc2ccoc2c1)C(CC(Cl)Cl)NCC ; C=C(c1ccc2ccoc2c1)C(CC(F)(F)F)NCC ; C=C(c1ccc2ccoc2c1)C(CC(Cl)(Cl)Cl)NCC]

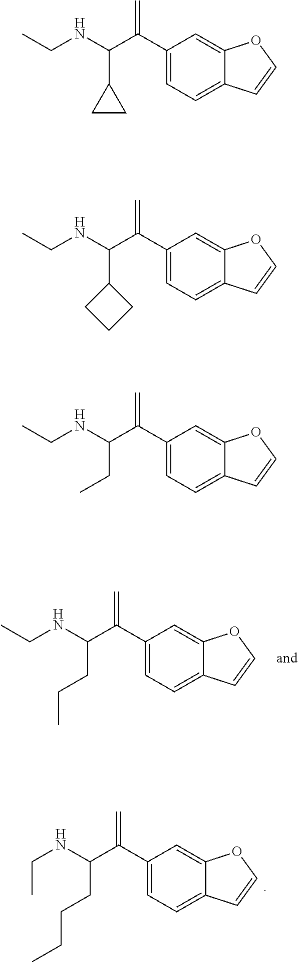 [SMILES: C=C(c1ccc2ccoc2c1)C(NCC)C1CC1 ; C=C(c1ccc2ccoc2c1)C(NCC)C1CCC1 ; C=C(c1ccc2ccoc2c1)C(CC)NCC ; C=C(c1ccc2ccoc2c1)C(CCC)NCC ; C=C(c1ccc2ccoc2c1)C(CCCC)NCC]

In certain embodiments, the salt or mixture of salts of the present invention is of a

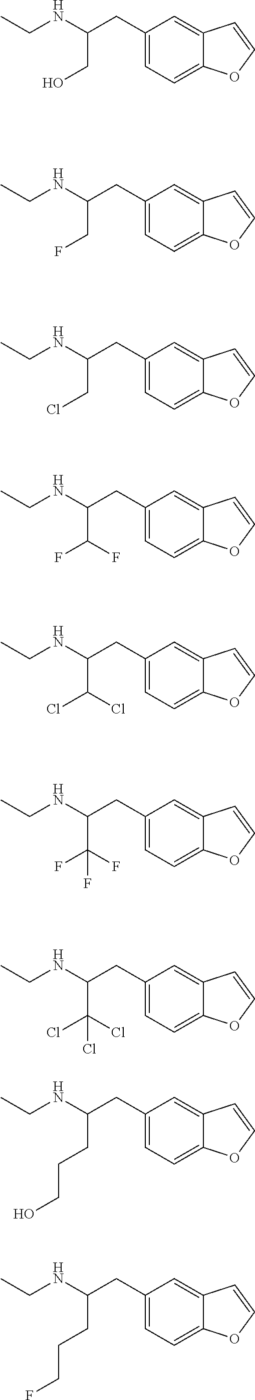

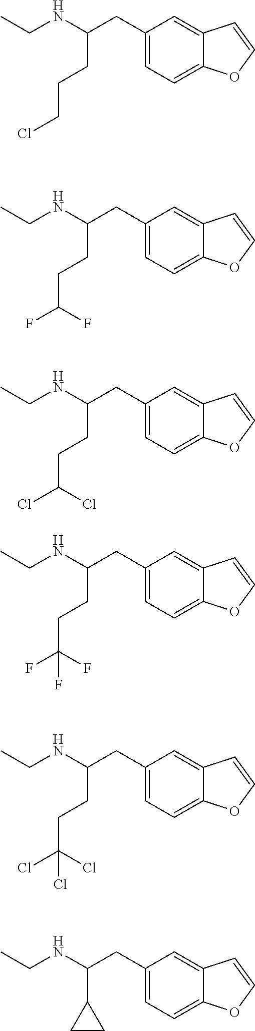 [SMILES: CCNC(CCCCl)Cc1ccc2occc2c1 ; CCNC(CCC(F)F)Cc1ccc2occc2c1 ; CCNC(CCC(Cl)Cl)Cc1ccc2occc2c1 ; CCNC(CCC(F)(F)F)Cc1ccc2occc2c1 ; CCNC(CCC(Cl)(Cl)Cl)Cc1ccc2occc2c1 ; CCNC(Cc1ccc2occc2c1)C1CC1]

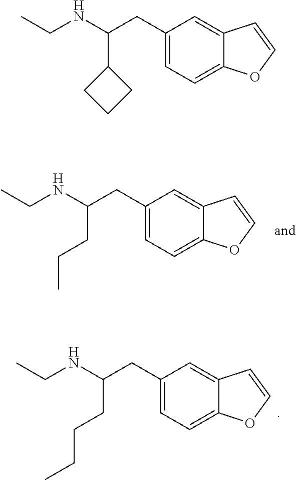 [SMILES: CCNC(Cc1ccc2occc2c1)C1CCC1 ; CCCC(Cc1ccc2occc2c1)NCC ; CCCCC(Cc1ccc2occc2c1)NCC]

In certain embodiments, the salt or mixture of salts of the present invention is of a compound selected from:

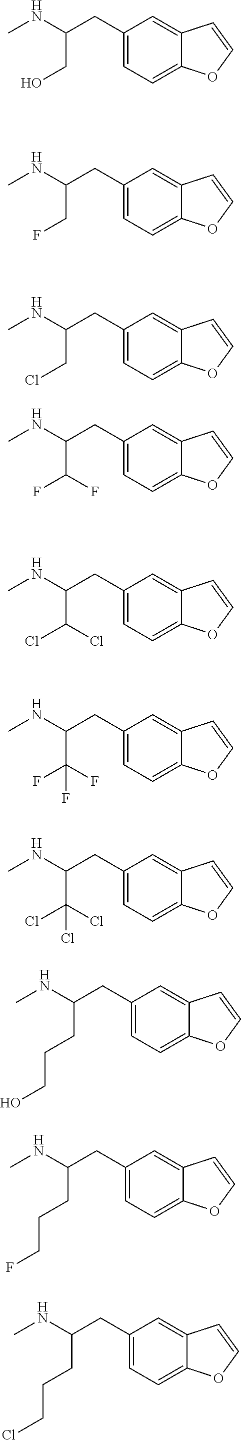

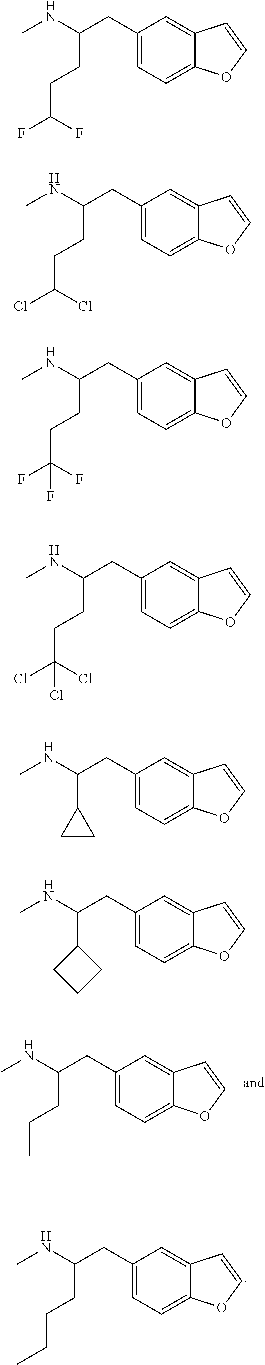

In certain embodiments, the salt or mixture of salts of the present invention is of a

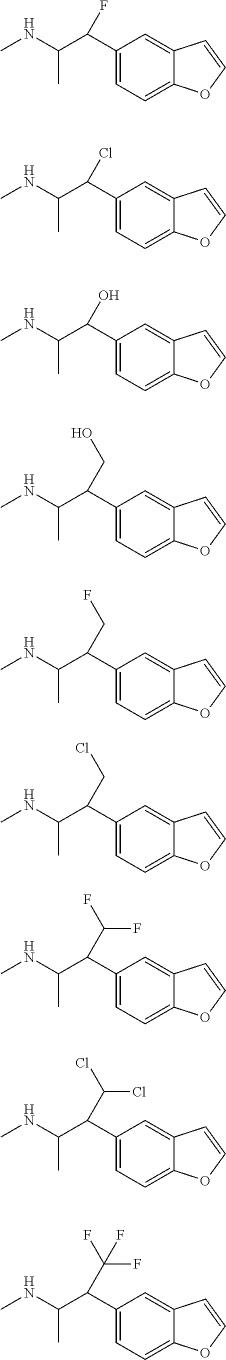

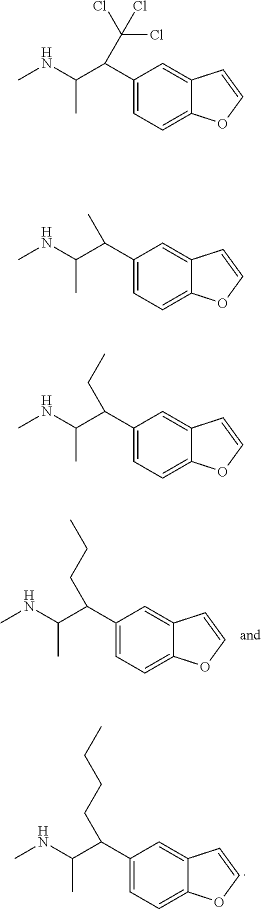 [SMILES: CNC(C)C(c1ccc2occc2c1)C(Cl)(Cl)Cl ; CNC(C)C(C)c1ccc2occc2c1 ; CCC(c1ccc2occc2c1)C(C)NC ; CCCC(c1ccc2occc2c1)C(C)NC ; CCCCC(c1ccc2occc2c1)C(C)NC]

In certain embodiments, the salt or mixture of salts of the present invention is of a compound selected from:

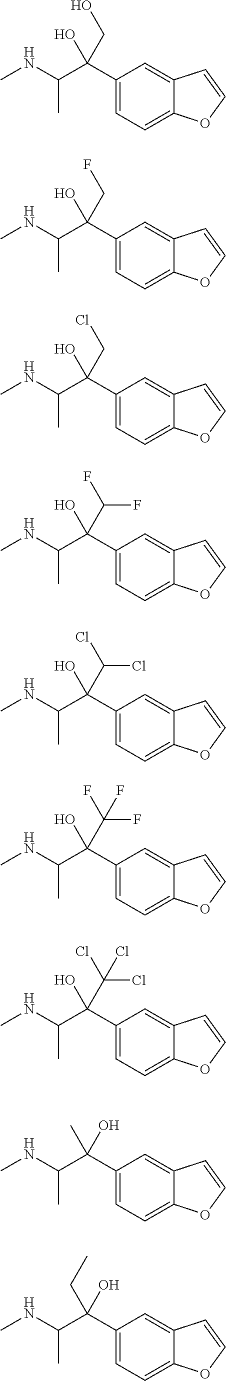

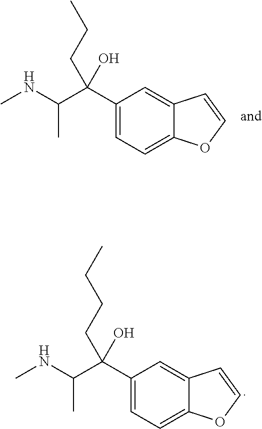 [SMILES: CCCC(O)(c1ccc2occc2c1)C(C)NC ; CCCCC(O)(c1ccc2occc2c1)C(C)NC]

In certain embodiments, the salt or mixture of salts of the present invention is of a

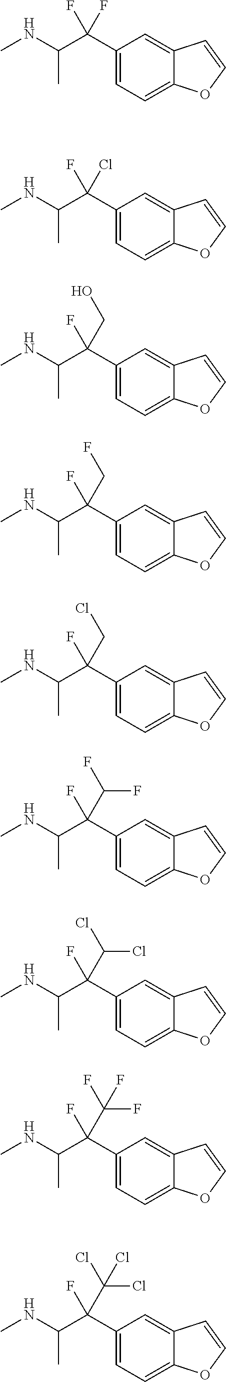

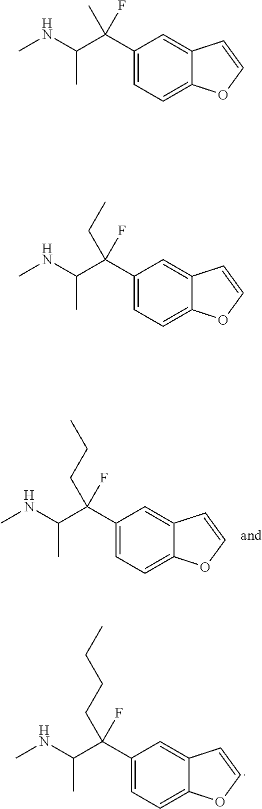 [SMILES: CNC(C)C(C)(F)c1ccc2occc2c1 ; CCC(F)(c1ccc2occc2c1)C(C)NC ; CCCC(F)(c1ccc2occc2c1)C(C)NC ; CCCCC(F)(c1ccc2occc2c1)C(C)NC]

In certain embodiments, the salt or mixture of salts of the present invention is of a

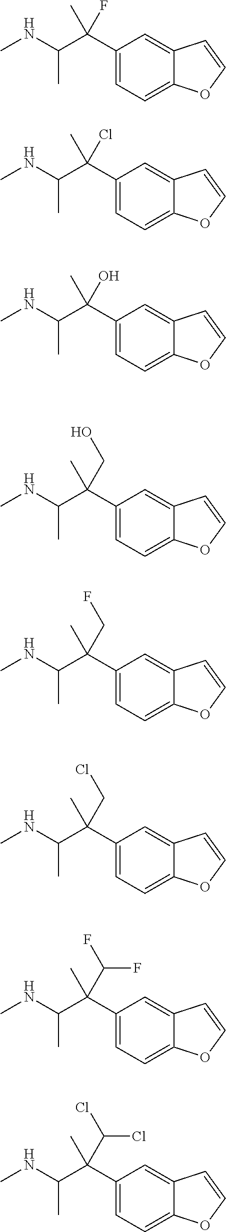

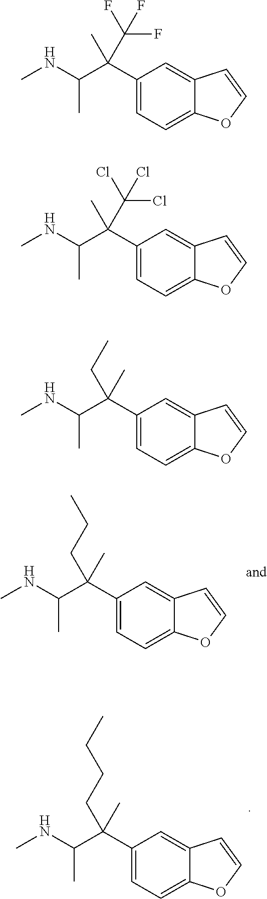 [SMILES: CNC(C)C(C)(c1ccc2occc2c1)C(F)(F)F ; CNC(C)C(C)(c1ccc2occc2c1)C(Cl)(Cl)Cl ; CCC(C)(c1ccc2occc2c1)C(C)NC ; CCCC(C)(c1ccc2occc2c1)C(C)NC ; CCCCC(C)(c1ccc2occc2c1)C(C)NC]

In certain embodiments, the salt or mixture of salts of the present invention is of a

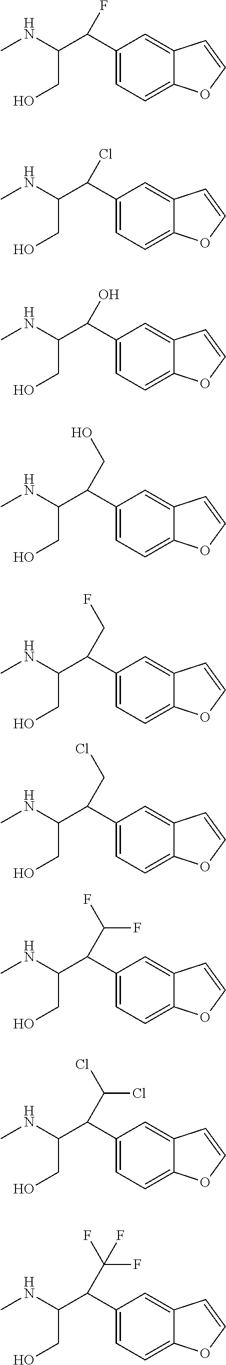

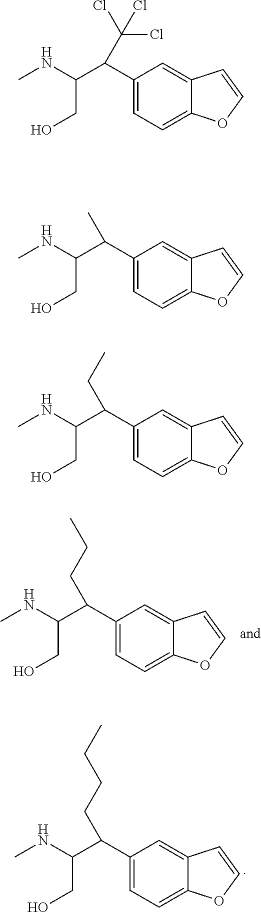 [SMILES: CNC(CO)C(c1ccc2occc2c1)C(Cl)(Cl)Cl ; CNC(CO)C(C)c1ccc2occc2c1 ; CCC(c1ccc2occc2c1)C(CO)NC ; CCCC(c1ccc2occc2c1)C(CO)NC ; CCCCC(c1ccc2occc2c1)C(CO)NC]

In certain embodiments, the salt or mixture of salts of the present invention is of a

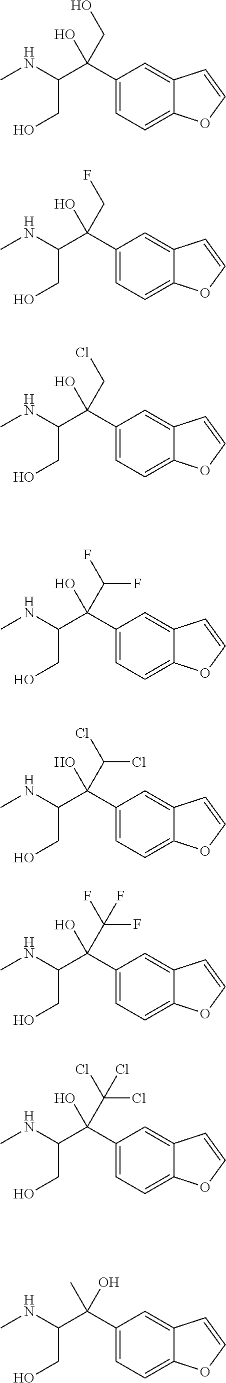

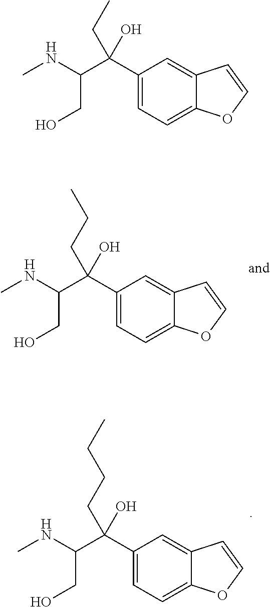 [SMILES: CCC(O)(c1ccc2occc2c1)C(CO)NC ; CCCC(O)(c1ccc2occc2c1)C(CO)NC ; CCCCC(O)(c1ccc2occc2c1)C(CO)NC]

In certain embodiments, the salt or mixture of salts of the present invention is of a

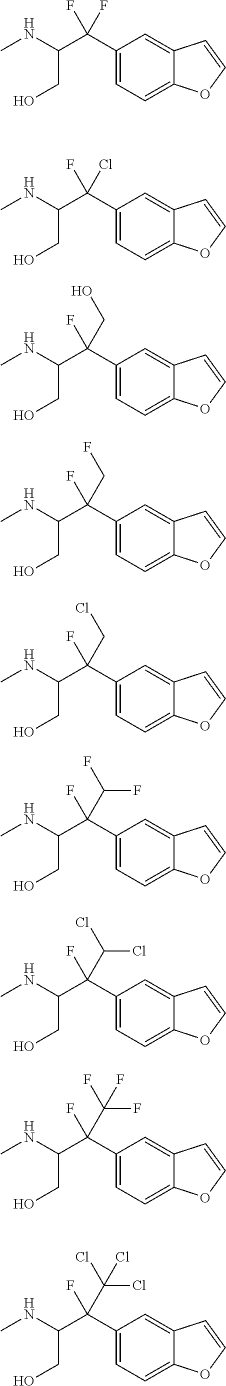

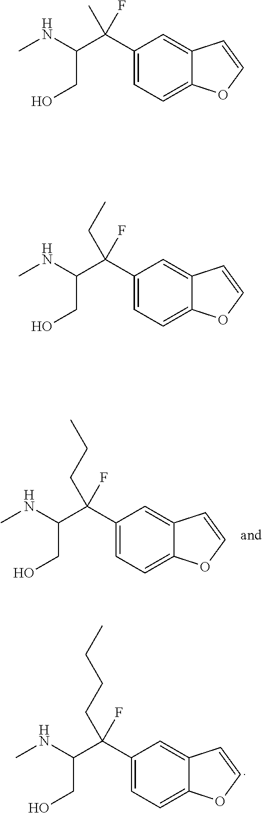 [SMILES: CNC(CO)C(C)(F)c1ccc2occc2c1 ; CCC(F)(c1ccc2occc2c1)C(CO)NC ; CCCC(F)(c1ccc2occc2c1)C(CO)NC ; CCCCC(F)(c1ccc2occc2c1)C(CO)NC]

In certain embodiments, the salt or mixture of salts of the present invention is of a

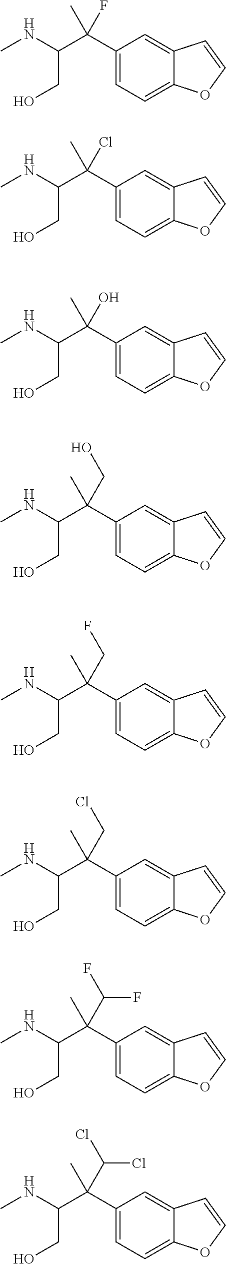

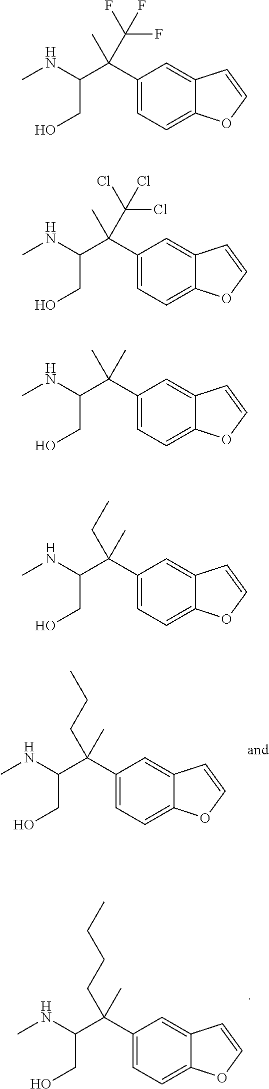 [SMILES: CNC(CO)C(C)(c1ccc2occc2c1)C(F)(F)F ; CNC(CO)C(C)(c1ccc2occc2c1)C(Cl)(Cl)Cl ; CNC(CO)C(C)(C)c1ccc2occc2c1 ; CCC(C)(c1ccc2occc2c1)C(CO)NC ; CCCC(C)(c1ccc2occc2c1)C(CO)NC ; CCCCC(C)(c1ccc2occc2c1)C(CO)NC]

In certain embodiments, the salt or mixture of salts of the present invention is of a

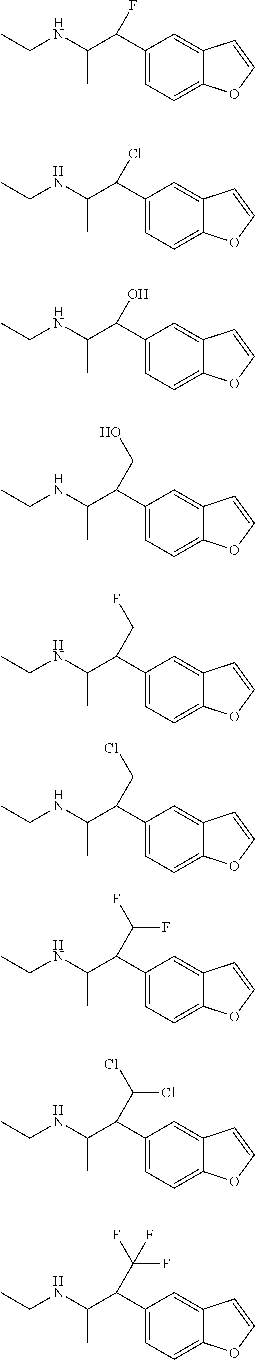

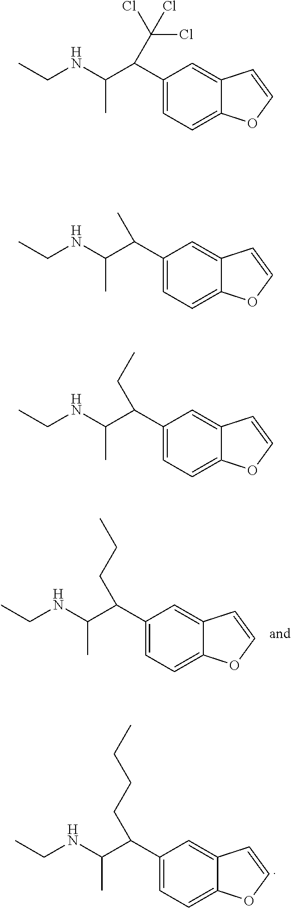 [SMILES: CCNC(C)C(c1ccc2occc2c1)C(Cl)(Cl)Cl ; CCNC(C)C(C)c1ccc2occc2c1 ; CCNC(C)C(CC)c1ccc2occc2c1 ; CCCC(c1ccc2occc2c1)C(C)NCC ; CCCCC(c1ccc2occc2c1)C(C)NCC]

In certain embodiments, the salt or mixture of salts of the present invention is of a

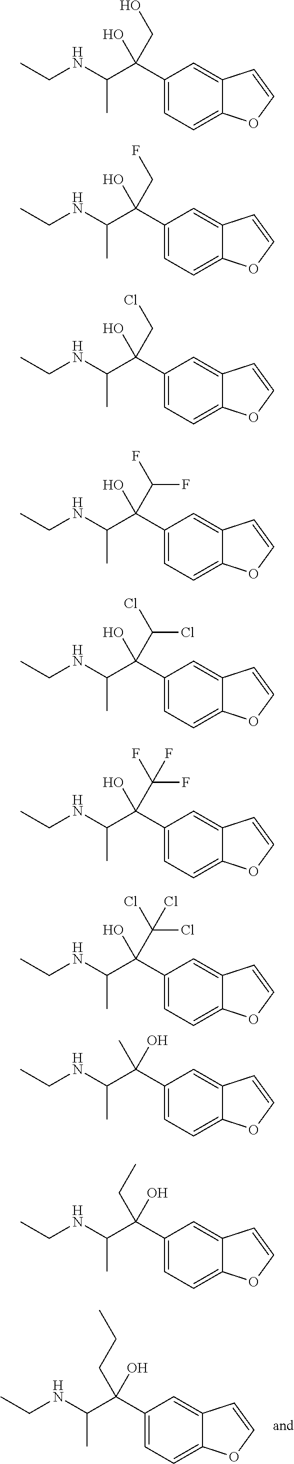

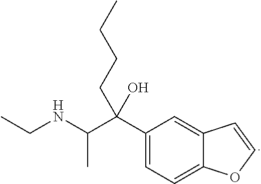 [SMILES: CCCCC(O)(c1ccc2occc2c1)C(C)NCC]

In certain embodiments, the salt or mixture of salts of the present invention is of a

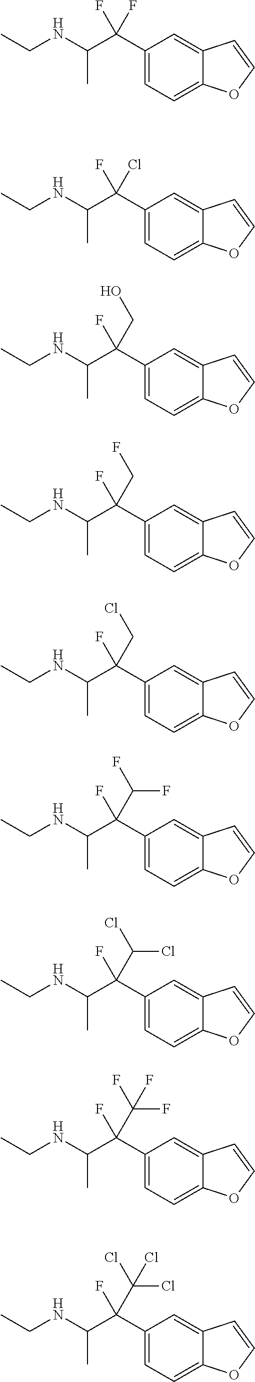

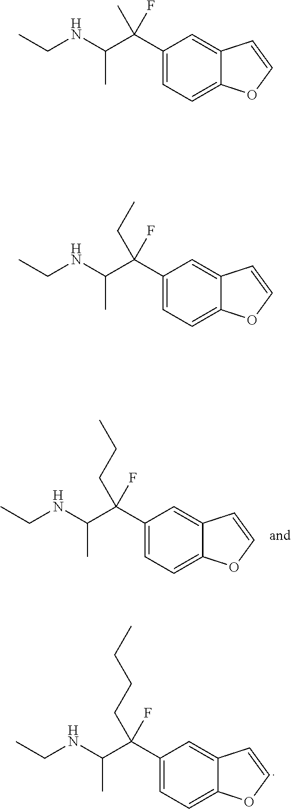 [SMILES: CCNC(C)C(C)(F)c1ccc2occc2c1 ; CCNC(C)C(F)(CC)c1ccc2occc2c1 ; CCCC(F)(c1ccc2occc2c1)C(C)NCC ; CCCCC(F)(c1ccc2occc2c1)C(C)NCC]

In certain embodiments, the salt or mixture of salts of the present invention is of a

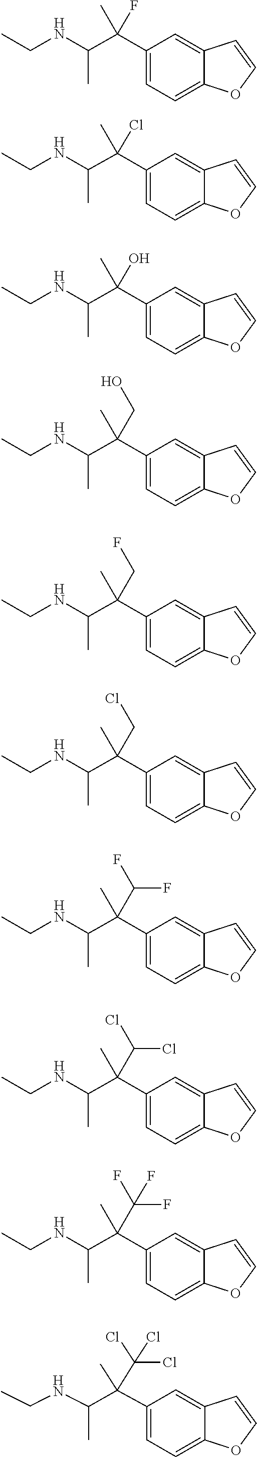

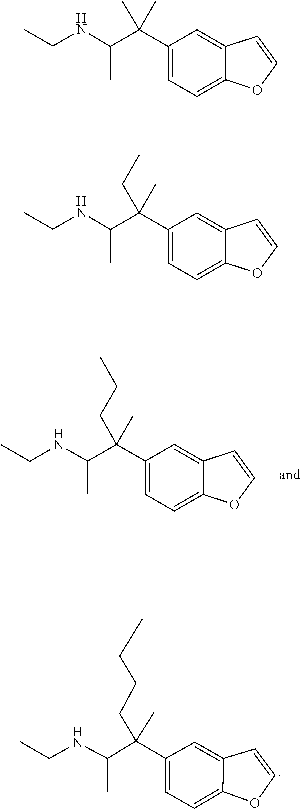 [SMILES: CCNC(C)C(C)(C)c1ccc2occc2c1 ; CCNC(C)C(C)(CC)c1ccc2occc2c1 ; CCCC(C)(c1ccc2occc2c1)C(C)NCC ; CCCCC(C)(c1ccc2occc2c1)C(C)NCC]

In certain embodiments, the salt or mixture of salts of the present invention is of a

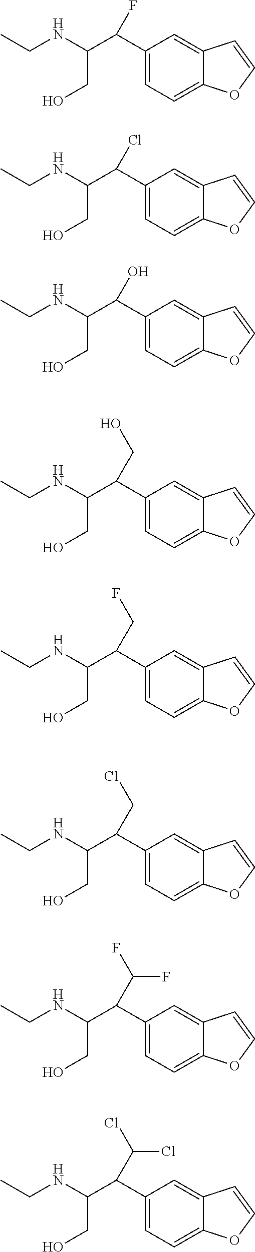

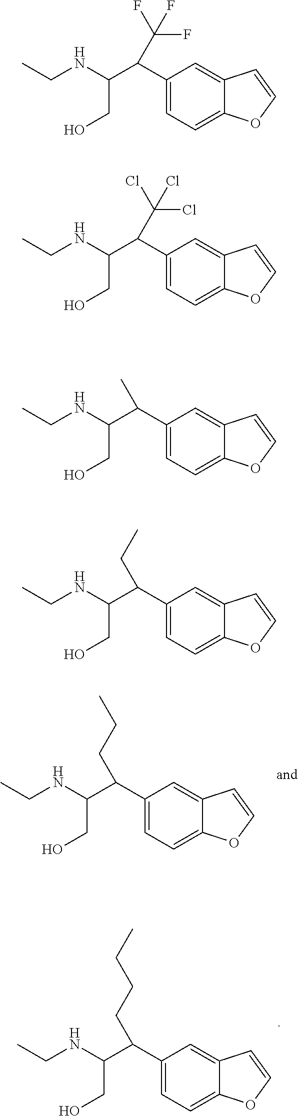

In certain embodiments, the salt or mixture of salts of the present invention is of a

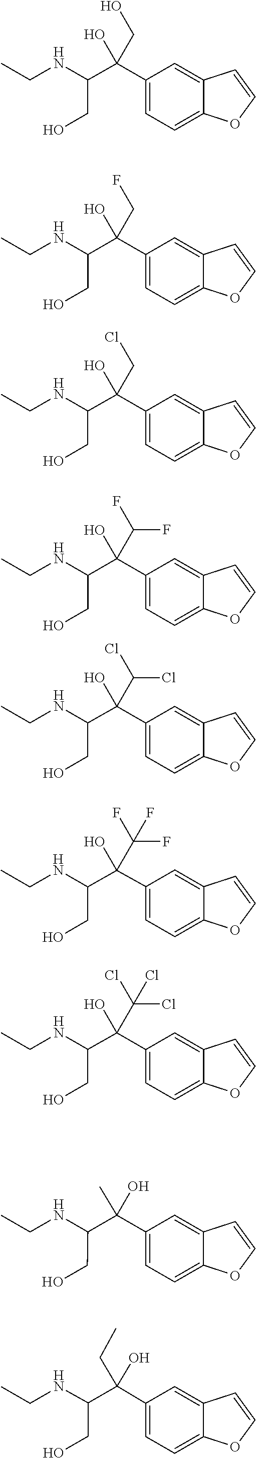

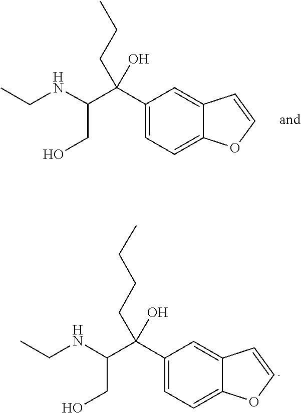 [SMILES: CCCC(O)(c1ccc2occc2c1)C(CO)NCC ; CCCCC(O)(c1ccc2occc2c1)C(CO)NCC]

In certain embodiments, the salt or mixture of salts of the present invention is of a

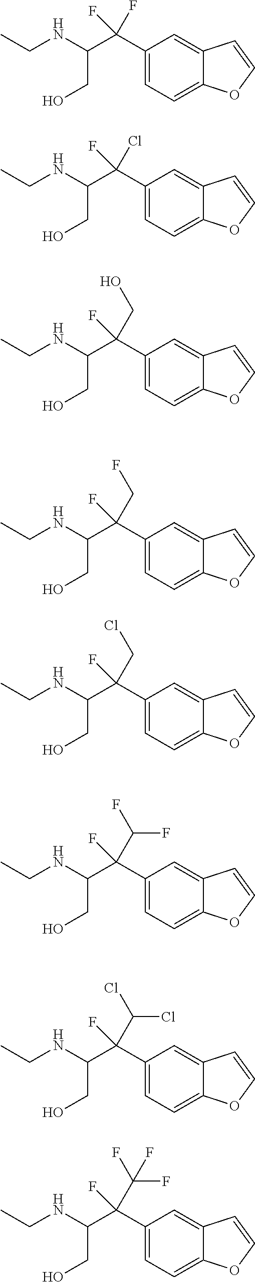

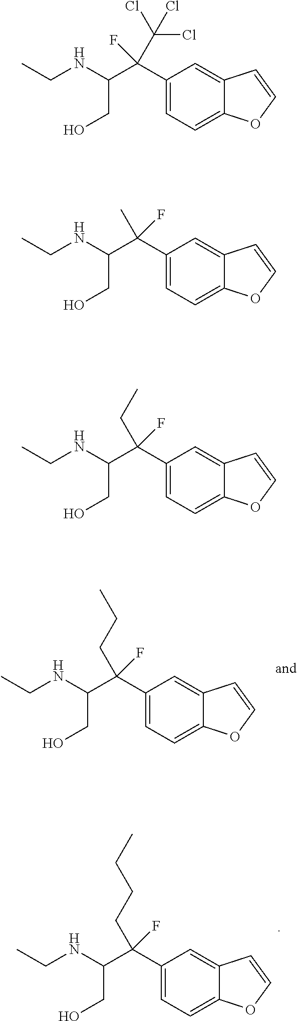 [SMILES: CCNC(CO)C(F)(c1ccc2occc2c1)C(Cl)(Cl)Cl ; CCNC(CO)C(C)(F)c1ccc2occc2c1 ; CCNC(CO)C(F)(CC)c1ccc2occc2c1 ; CCCC(F)(c1ccc2occc2c1)C(CO)NCC ; CCCCC(F)(c1ccc2occc2c1)C(CO)NCC]

In certain embodiments, the salt or mixture of salts of the present invention is of a

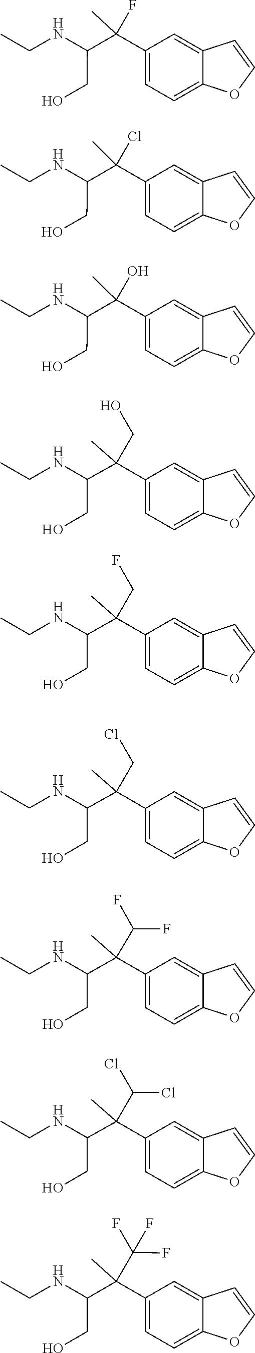

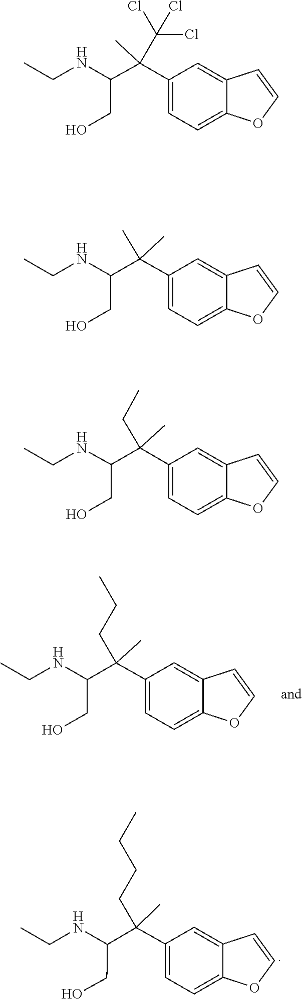 [SMILES: CCNC(CO)C(C)(c1ccc2occc2c1)C(Cl)(Cl)Cl ; CCNC(CO)C(C)(C)c1ccc2occc2c1 ; CCNC(CO)C(C)(CC)c1ccc2occc2c1 ; CCCC(C)(c1ccc2occc2c1)C(CO)NCC ; CCCCC(C)(c1ccc2occc2c1)C(CO)NCC]

Certain compounds of the invention may also exist in several tautomeric forms including the enol form, the keto form, and mixtures thereof. Accordingly, the chemical structures depicted herein encompass all possible tautomeric forms of the illustrated compounds. Keto-enol tautomerism, for example, is the reversible transfer of a hydrogen from the alpha carbon adjacent to a carbonyl group followed by a double bond transfer. In solution, compounds will spontaneously undergo a kinetic transformation from one tautomer to the other until equilibrium is reached, generally strongly favoring the keto tautomer over the enol tautomer, but dependent on factors such as solvent, pH, and temperature. Keto and enol tautomers may have distinguishable physicochemical properties; however, because they will interconvert in solution, reference to a compound in its keto form (e.g., where Q is

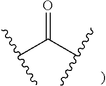 [SMILES: *C(*)=O]

will be understood to refer to and include the compound in its enol form (e.g., where Q is

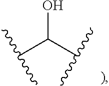 [SMILES: *C(*)O]

unless context clearly indicates otherwise. The compounds may also exist as ring-chain tautomers, as discussed below.

### Preparation of Enantiomeric Compounds

Various methods are known in the art for preparing optically active forms and determining activity. Such methods include standard processes described herein and other similar assays which are well known in the art. Examples of methods that can be used to obtain optical isomers of the compounds according to the present disclosure include but are not limited to the following:
a) physical separation of crystals whereby macroscopic crystals of the individual enantiomers are manually separated. This technique may particularly be used if crystals of the separate enantiomers exist (i.e., the material is a conglomerate), and the crystals are visually distinct;
b) simultaneous crystallization whereby the individual enantiomers are separately crystallized from a solution of the racemate, possible only if the latter is a conglomerate in the solid state;
c) enzymatic resolutions whereby partial or complete separation of a racemate by virtue of differing rates of reaction for the enantiomers with an enzyme;
d) enzymatic asymmetric synthesis, a synthetic technique whereby at least one step of the synthesis uses an enzymatic reaction to obtain an enantiomerically pure or enriched synthetic precursor of the desired enantiomer;
e) chemical asymmetric synthesis whereby the desired enantiomer is synthesized from an achiral precursor under conditions that produce asymmetry (i.e., chirality) in the product, which may be achieved using chiral catalysts or chiral auxiliaries;
f) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
g) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;
h) kinetic resolutions comprising partial or complete resolution of a racemate (or of a further resolution of a partially resolved compound) by virtue of unequal reaction rates of the enantiomers with a chiral, enantiomerically enriched reagent or catalyst under kinetic conditions;
i) enantiospecific synthesis from enantiomerically enriched precursors whereby the desired enantiomer is obtained from non-chiral starting materials and where the stereochemical integrity is not or is only minimally compromised over the course of the synthesis;
j) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;
k) chiral gas chromatography whereby the racemate is volatilized and enantiomers are separated by virtue of their differing interactions in the gaseous mobile phase with a column containing a fixed enantiomerically enriched chiral adsorbent phase;
l) extraction with chiral solvents whereby the enantiomers are separated by virtue of preferential dissolution of one enantiomer into a particular chiral solvent; and
m) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the enantiomerically enriched chiral nature of the membrane, which allows only one enantiomer of the racemate to pass through.

Where diastereomers exist, the compounds can be used in any diastereomeric form or mixture of forms that provides the appropriate therapeutic effect for the patient, as taught herein. Therefore, in one embodiment, the compounds of the present invention can be administered in a racemic mixture, as the R-enantiomer, as the S-enantiomer, or as an enantiomerically enriched mixture, or a diastereomeric form.

The following compounds indicate where primary stereocenters exist when the designated R group is not hydrogen. In certain embodiments, the enantiomers of the present invention include:

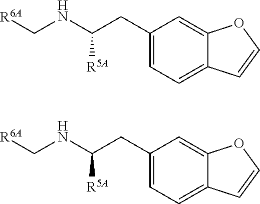

wherein R^{5A} is not hydrogen.

In certain embodiments, the enantiomers of the present invention include:

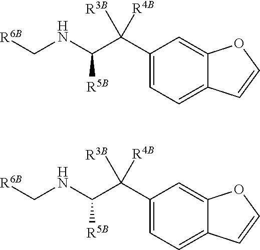

wherein R^{5B} is not hydrogen.

In certain embodiments, the enantiomers of the present invention include:

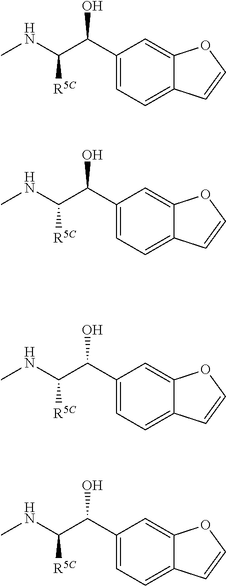

wherein R^{5C} is not hydrogen.

In certain embodiments, the enantiomers of the present invention include:

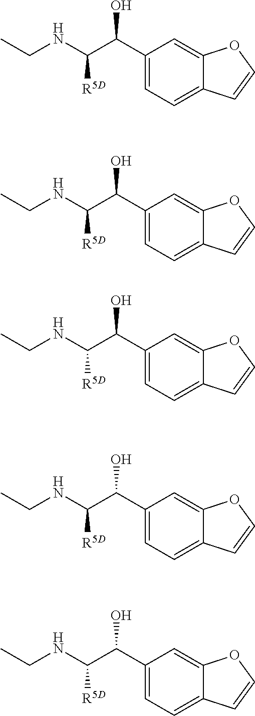

wherein R^{5D} is not hydrogen.

In certain embodiments, the enantiomers of the present invention include:

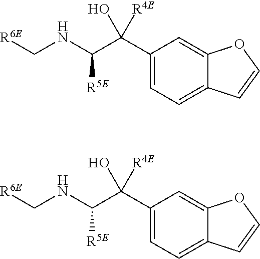

wherein R^{5E} is not hydrogen.

In certain embodiments, the enantiomers of the present invention include:

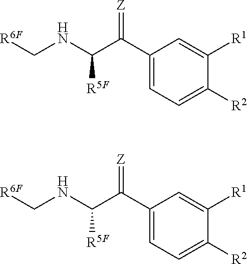

wherein R^{5F} is not hydrogen.

In certain embodiments, the enantiomers of the present invention include:

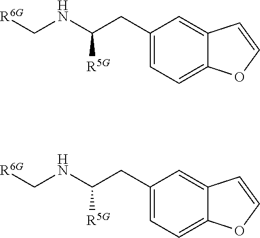

wherein R^{5G} is not hydrogen.

In certain embodiments, the enantiomers of the present invention include:

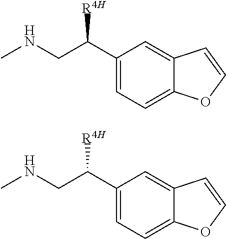

wherein R^{4H} is not hydrogen.

In certain embodiments, the enantiomers of the present invention include:

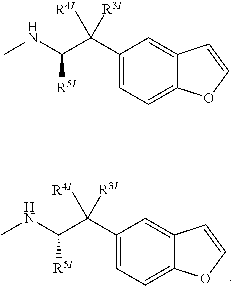

In certain embodiments, the enantiomers of the present invention include:

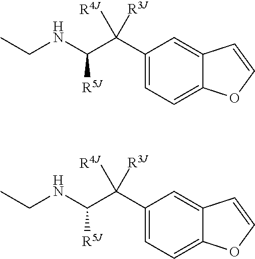

wherein R^{5J} is not hydrogen.

In certain embodiments, the enantiomers of the present invention include:

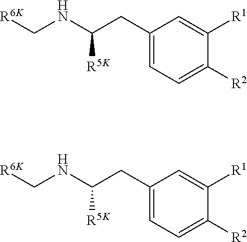

wherein R^{5K} is not hydrogen.

In certain embodiments, the enantiomers of the present invention include:

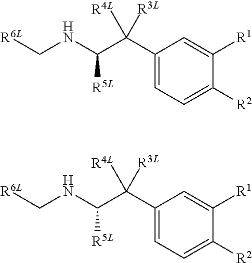

wherein R^{5L} is not hydrogen.

In certain embodiments, the enantiomers of the present invention include:

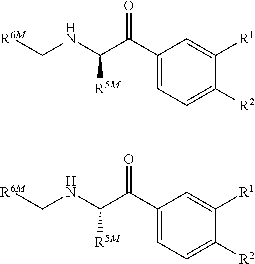

wherein R^{5M} is not hydrogen.

### Enantiomerically Enriched Pharmaceutical Compositions

Chiral compounds of the invention may be prepared by chiral chromatography from the racemic or enantiomerically enriched free amine. Pharmaceutically acceptable salts of chiral compounds may be prepared from fractional crystallization of salts from a racemic or an enantiomerically enriched free amine and a chiral acid. Alternatively, the free amine may be reacted with a chiral auxiliary and the enantiomers separated by chromatography followed by removal of the chiral auxiliary to regenerate the free amine. Furthermore, separation of enantiomers may be performed at any convenient point in the synthesis of the compounds of the invention. The compounds of the invention may also be prepared using a chiral synthesis.

An enantiomerically enriched mixture is a mixture that contains one enantiomer in a greater amount than the other. An enantiomerically enriched mixture of an S-enantiomer contains at least 55% of the S-enantiomer, and more typically at least about 60%, 65%, 70%, 75%, 80%, 85%, 90%, 95% of the S-enantiomer. An enantiomerically enriched mixture of an R-enantiomer contains at least 55% of the R-enantiomer, more typically at least about 55%, 60%, 65%, 70%, 75%, 80%, 85%, 90%, 95% of the R-enantiomer.

In one embodiment, enantiomerically enriched mixtures that have a greater amount of the R-enantiomer maximize nicotinic-receptor-dependent therapeutic effects. In one embodiment, enantiomerically enriched mixtures that have a greater amount of the S-enantiomer maximize serotonin-receptor-dependent therapeutic effects. Accordingly, in one embodiment, an enantiomerically enriched mixture of S-5-MAPB or an enantiomerically enriched mixture of S-6-MAPB maximize serotonin-receptor-dependent therapeutic effects and minimized unwanted nicotinic effects when administered to a host in need thereof, for example a mammal, including a human. In another embodiment, an enantiomerically enriched mixture of R-5-MAPB or an enantiomerically enriched mixture of R-6-MAPB maximize nicotinic-receptor-dependent therapeutic effects while minimizing unwanted effects, when administered to a host in need thereof, including a mammal, for example, a human.

Non-limiting examples of unwanted effects that can be minimized include psychoactive effects (such as excess stimulation or sedation), physiological effects (such as transient hypertension or appetite suppression), toxic effects (such as to the brain or liver), effects contributing to abuse liability (such as euphoria or dopamine release), and other side effects.

One aspect of the present invention is a balanced mixture of S-5-MAPB and R-5-MAPB (not the racemate) or a balanced mixture of S-6-MAPB and R-6-MAPB (not the racemate) that achieves a predetermined combination of serotonin-receptor-dependent therapeutic effects and nicotinic-receptor-dependent therapeutic effects.

In certain embodiments, pharmaceutical compositions of enantiomerically enriched preparations of 5-MAPB or 6-MAPB are provided. In one embodiment, the pharmaceutical composition is enriched with S-5-MAPB. In one embodiment, the pharmaceutical composition is enriched with R-5-MAPB. In one embodiment, the pharmaceutical composition is enriched with S-6-MAPB. In one embodiment, the pharmaceutical composition is enriched with R-6-MAPB.

Example 1 below provides a non-limiting example for the preparation of certain enantiomerically enriched preparations of 5-MAPB (i.e., comprising S-5-MAPB and R-5-MAPB). Enantiomerically enriched preparations of 6-MAPB (i.e., S-6-MAPB, R-6-MAPB) can be similarly produced using racemic 6-MAPB HCl.

Particular embodiments for pharmaceutical compositions, including enantiomerically enriched pharmaceutical compositions, of the present invention include:
a) S-5-MAPB;
b) R-5-MAPB;
c) S-6-MAPB;
d) R-6-MAPB;
e) Embodiments (a)-(d) wherein the compound is a free base;
f) Embodiments (a)-(d) wherein the compound is a salt;
g) Embodiment (f) wherein the compound is the hydrochloride salt;
h) A mixture of S-5-MAPB, R-5-MAPB and there is more S-enantiomer than R-enantiomer;
i) A mixture of S-5-MAPB, R-5-MAPB and there is less S-enantiomer than R-enantiomer;
j) A mixture of S-6-MAPB, R-6-MAPB and there is more S-enantiomer than R-enantiomer;
k) A mixture of S-6-MAPB, R-6-MAPB and there is less S-enantiomer than R-enantiomer;
l) A mixture of S-5-MAPB, R-5-MAPB and about 65% is the S-enantiomer while about 35% is the R-enantiomer;
m) A mixture of S-5-MAPB, R-5-MAPB and greater than 65% is the S-enantiomer while less than 35% is the R-enantiomer;
n) A mixture of S-5-MAPB, R-5-MAPB and greater than 90% is the S-enantiomer while less than 10% is the R-enantiomer;
o) A mixture of S-5-MAPB, R-5-MAPB and about 35% is the S-enantiomer while about 65% is the R-enantiomer;
p) A mixture of S-5-MAPB, R-5-MAPB and less than 35% is the S-enantiomer while greater than 65% is the R-enantiomer;
q) A mixture of S-5-MAPB, R-5-MAPB and less than 10% is the S-enantiomer while greater than 90% is the R-enantiomer;
r) A mixture of S-6-MAPB, R-6-MAPB and about 65% is the S-enantiomer while about 35% is the R-enantiomer;
s) A mixture of S-6-MAPB, R-6-MAPB and greater than 65% is the S-enantiomer while less than 35% is the R-enantiomer;
t) A mixture of S-6-MAPB, R-6-MAPB and greater than 90% is the S-enantiomer while less than 10% is the R-enantiomer;
u) A mixture of S-6-MAPB, R-6-MAPB and 35% or less is the S-enantiomer while 65% or more is the R-enantiomer;
v) A mixture of S-6-MAPB, R-6-MAPB and about 35% is the S-enantiomer while about 65% is the R-enantiomer; and
w) A mixture of S-6-MAPB, R-6-MAPB and less than 10% is the S-enantiomer while greater than 90% is the R-enantiomer.
x) S-5-MBPB;
y) R-5-MBPB;
z) S-6-MBPB;
aa) R-6-MBPB;
bb) Embodiments (x)-(aa) wherein the compound is a free base;
cc) Embodiments (x)-(aa) wherein the compound is a salt;
dd) Embodiment (cc) wherein the compound is the hydrochloride salt;
ee) A mixture of S-5-MBPB, R-5-MBPB and there is more S-enantiomer than R-enantiomer;
ff) A mixture of S-5-MBPB, R-5-MBPB and there is less S-enantiomer than R-enantiomer;
gg) A mixture of S-6-MBPB, R-6-MBPB and there is more S-enantiomer than R-enantiomer;
hh) A mixture of S-6-MBPB, R-6-MBPB and there is less S-enantiomer than R-enantiomer;
ii) A mixture of S-5-MBPB, R-5-MBPB and about 65% is the S-enantiomer while about 35% is the R-enantiomer;
jj) A mixture of S-5-MBPB, R-5-MBPB and greater than about 65% is the S-enantiomer while less than about 35% is the R-enantiomer;
kk) A mixture of S-5-MBPB, R-5-MBPB and greater than about 90% is the S-enantiomer while less than about 10% is the R-enantiomer;
ll) A mixture of S-5-MBPB, R-5-MBPB and about 35% is the S-enantiomer while about 65% is the R-enantiomer;
mm) A mixture of S-5-MBPB, R-5-MBPB and less than about 35% is the S-enantiomer while greater than about 65% is the R-enantiomer;
nn) A mixture of S-5-MBPB, R-5-MBPB and less than about 10% is the S-enantiomer while greater than about 90% is the R-enantiomer;
oo) A mixture of S-6-MBPB, R-6-MBPB and about 65% is the S-enantiomer while about 35% is the R-enantiomer;
pp) A mixture of S-6-MBPB, R-6-MBPB and greater than about 65% is the S-enantiomer while less than about 35% is the R-enantiomer;
qq) A mixture of S-6-MBPB, R-6-MBPB and greater than about 90% is the S-enantiomer while less than about 10% is the R-enantiomer;
rr) A mixture of S-6-MBPB, R-6-MBPB and about 35% or less is the S-enantiomer while about 65% or more is the R-enantiomer;
ss) A mixture of S-6-MBPB, R-6-MBPB and about 35% is the S-enantiomer while about 65% is the R-enantiomer; and
tt) A mixture of S-6-MBPB, R-6-MBPB and less than about 10% is the S-enantiomer while greater than about 90% is the R-enantiomer.
uu) S-Bk-5-MAPB;
vv) R-Bk-5-MAPB;
ww) S-Bk-6-MAPB;
xx) R-Bk-6-MAPB;
yy) Embodiments (uu)-(xx) wherein the compound is a free base;
zz) Embodiments (uu)-(xx) wherein the compound is a salt;
aaa) Embodiment (zz) wherein the compound is the hydrochloride salt;
bbb) A mixture of S-Bk-5-MAPB, R-Bk-5-MAPB and there is more S-enantiomer than R-enantiomer;
ccc) A mixture of S-Bk-5-MAPB, R-Bk-5-MAPB and there is less S-enantiomer than R-enantiomer;
ddd) A mixture of S-Bk-6-MAPB, R-Bk-6-MAPB and there is more S-enantiomer than R-enantiomer;
eee) A mixture of S-Bk-6-MAPB, R-Bk-6-MAPB and there is less S-enantiomer than R-enantiomer;
fff) A mixture of S-Bk-5-MAPB, R-Bk-5-MAPB and about 65% is the S-enantiomer while about 35% is the R-enantiomer;
ggg) A mixture of S-Bk-5-MAPB, R-Bk-5-MAPB and greater than about 65% is the S-enantiomer while less than about 35% is the R-enantiomer;
hhh) A mixture of S-Bk-5-MAPB, R-Bk-5-MAPB and greater than about 90% is the S-enantiomer while less than about 10% is the R-enantiomer;
iii) A mixture of S-Bk-5-MAPB, R-Bk-5-MAPB and about 35% is the S-enantiomer while about 65% is the R-enantiomer;
jjj) A mixture of S-Bk-5-MAPB, R-Bk-5-MAPB and less than about 35% is the S-enantiomer while greater than about 65% is the R-enantiomer;
kkk) A mixture of S-Bk-5-MAPB, R-Bk-5-MAPB and less than about 10% is the S-enantiomer while greater than about 90% is the R-enantiomer;
lll) A mixture of S-Bk-6-MAPB, R-Bk-6-MAPB and about 65% is the S-enantiomer while about 35% is the R-enantiomer;
mmm) A mixture of S-Bk-6-MAPB, R-Bk-6-MAPB and greater than about 65% is the S-enantiomer while less than about 35% is the R-enantiomer;
nnn) A mixture of S-Bk-6-MAPB, R-Bk-6-MAPB and greater than about 90% is the S-enantiomer while less than about 10% is the R-enantiomer;
ooo) A mixture of S-Bk-6-MAPB, R-Bk-6-MAPB and about 35% or less is the S-enantiomer while about 65% or more is the R-enantiomer;
ppp) A mixture of S-Bk-6-MAPB, R-Bk-6-MAPB and about 35% is the S-enantiomer while about 65% is the R-enantiomer; and
qqq) A mixture of S-Bk-6-MAPB, R-Bk-6-MAPB and less than about 10% is the S-enantiomer while greater than about 90% is the R-enantiomer.
rrr) S-Bk-5-MBPB;
sss) R-Bk-5-MBPB;
ttt) S-Bk-6-MBPB;
uuu) R-Bk-6-MBPB;
VVV) Embodiments (rrr)-(uuu) wherein the compound is a free base;
www) Embodiments (rrr)-(uuu) wherein the compound is a salt;
xxx) Embodiment (www) wherein the compound is the hydrochloride salt;
yyy) A mixture of S-Bk-5-MBPB, R-Bk-5-MBPB and there is more S-enantiomer than R-enantiomer;
zzz) A mixture of S-Bk-5-MBPB, R-Bk-5-MBPB and there is less S-enantiomer than R-enantiomer;
aaaa) A mixture of S-Bk-6-MBPB, R-Bk-6-MBPB and there is more S-enantiomer than R-enantiomer;
bbbb) A mixture of S-Bk-6-MBPB, R-Bk-6-MBPB and there is less S-enantiomer than R-enantiomer;
cccc) A mixture of S-Bk-5-MBPB, R-Bk-5-MBPB and about 65% is the S-enantiomer while about 35% is the R-enantiomer;
dddd) A mixture of S-Bk-5-MBPB, R-Bk-5-MBPB and greater than about 65% is the S-enantiomer while less than about 35% is the R-enantiomer;
eeee) A mixture of S-Bk-5-MBPB, R-Bk-5-MBPB and greater than about 90% is the S-enantiomer while less than about 10% is the R-enantiomer;
ffff) A mixture of S-Bk-5-MBPB, R-Bk-5-MBPB and about 35% is the S-enantiomer while about 65% is the R-enantiomer;
gggg) A mixture of S-Bk-5-MBPB, R-Bk-5-MBPB and less than about 35% is the S-enantiomer while greater than about 65% is the R-enantiomer;
hhhh) A mixture of S-Bk-5-MBPB, R-Bk-5-MBPB and less than about 10% is the S-enantiomer while greater than about 90% is the R-enantiomer;
iiii) A mixture of S-Bk-6-MBPB, R-Bk-6-MBPB and about 65% is the S-enantiomer while about 35% is the R-enantiomer;
jjjj) A mixture of S-Bk-6-MBPB, R-Bk-6-MBPB and greater than about 65% is the S-enantiomer while less than about 35% is the R-enantiomer;
kkkk) A mixture of S-Bk-6-MBPB, R-Bk-6-MBPB and greater than about 90% is the S-enantiomer while less than about 10% is the R-enantiomer;
llll) A mixture of S-Bk-6-MBPB, R-Bk-6-MBPB and about 35% or less is the S-enantiomer while about 65% or more is the R-enantiomer;
mmmm) A mixture of S-Bk-6-MBPB, R-Bk-6-MBPB and about 35% is the S-enantiomer while about 65% is the R-enantiomer; and
nnnn) A mixture of S-Bk-6-MBPB, R-Bk-6-MBPB and less than about 10% is the S-enantiomer while greater than about 90% is the R-enantiomer.

It will be understood that the above embodiments and classes of embodiments can be combined to form additional preferred embodiments.

### III. 6-Mbpb Free Base and Salts

The present invention also provides the compound 6-MBPB as a free base or salt, compositions containing it, and methods to treat mental disorders and more generally central nervous disorders, as well as for mental enhancement. 6-MBPB provides unexpected advantageous pharmacological properties that are highly desirable as therapeutics for the treatment of mental disorders, particularly as psychotherapeutics and neurotherapeutics.

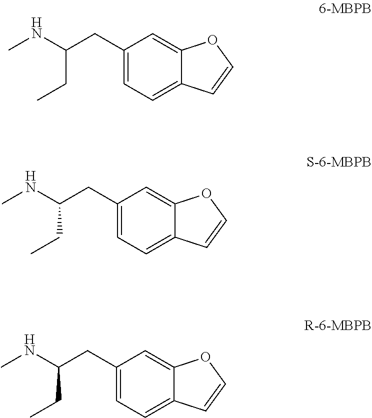

The embodiments of 6-MBPB of the invention are presented to meet the goal of assisting persons with mental disorders, who desire mental enhancement or suffer from other CNS disorders by providing milder therapeutics that are fast acting and that reduce the properties that decrease the patient experience, are counterproductive to the therapy or are undesirably toxic. One goal of the invention is to provide therapeutic compositions that increase empathy, sympathy, openness and acceptance of oneself and others, which can be taken if necessary, as part of therapeutic counseling sessions, or when necessary, episodically, or even consistently, as prescribed by a healthcare provider.

6-MBPB has a beneficial selectivity profile for neurotransmitter transporters compared to MDMA or other known entactogens. 6-MBPB is a 5-HT releaser and is only partial releaser of norepinephrine. Additionally, 6-MBPB enantiomers have differing effects on dopamine. While S-6-MBPB is a partial releaser of dopamine, R-6-MBPB is a dopamine uptake inhibitor. The inhibition of dopamine uptake creates a concentration-dependent ceiling on dopamine release that will lower the DAT to SERT ratio of compound at higher concentrations. This surprising combination is highly desirable. The balance of weakly activating NET (to reduce cardiovascular toxicity risk) and decreasing the DAT to SERT ratio with higher doses compared to typical entacotgens can to increase therapeutic effect relative to addictive liability and is a desirable feature for therapeutic compounds and compositions.

It has been discovered that 6-MBPB is a direct 5-HT_{1B} agonist. Very few substances are known that are 5-HT_{1B} agonists and also 5-HT releasers and these have significant toxicities. For example, meta-chlorophenylpiperazine (mCPP) is one example but is anxiogenic and induces headaches, limiting any clinical use. MDMA itself does not bind directly to the 5-HT_{1B} (Ray. 2010. PloS one, 5 (2), e9019). 5-HT_{1B} agonism is noteworthy because indirect stimulation of these receptors, secondary to elevated extracellular serotonin, has been hypothesized to be required for the prosocial effects of MDMA (Heifets et al. 2019. Science translational medicine, 11 (522)), while other aspects of entactogen effects have been attributed to monoamine release (e.g., Luethi & Liechti. 2020. Archives of toxicology, 94 (4), 1085-1133). Thus, the unique ratios of 5-HT_{1B} stimulation and monoamine release displayed by the disclosed compounds enable different profiles of therapeutic effects that appear not achieved by MDMA or other known entactogens.

Additionally, 6-MBPB shows a 5-HT receptor selectivity pattern that is important to therapeutic use. It has been surprisingly discovered that 6-MBPB is a poor agonist of 5-HT_{2A} but exhibits activity toward 5-HT_{1B}. Agonism of the 5-HT_{2A} receptor can cause labile mood, anxiety, and perceptual distortions, while agonism of 5-HT_{1B} is believed to be tied to the pro-social effects of entactogens.

6-MBPB lacks interactions with hTAAR1 (human trace amine-associated receptor), even at concentrations up to 60 μM. TAAR1 is an intracellular receptor expressed within the presynaptic terminal of monoamine neurons. MDMA and non-cathinone stimulants bind to and stimulate TAAR1 (e.g., Rickli, et al, 2015. British journal of pharmacology, 172 (13), pp. 3412-3425.), which decreases and shortens the acute therapeutic effects of these drugs, at least in part by causing internalization of the monoamine transporter (Underhill, & Amara. 2020. The FASEB Journal, 34 (S1), pp. 1-1). This internalization of monoamine transporter contributes to short-term tolerance (tachyphylaxis) to the entactogen and may further contribute to decreased mood and undesirable effects in the week after drug exposure. It is both advantageous and unexpected for an entactogen to lack effects at TAAR1.

In some embodiments, 6-MBPB, as a racemic mixture, enantiomerically enriched mixture or pure enantiomer has a duration of acute therapeutic effects that is longer than that of MDMA (reported to be 4.2 hours with a standard deviation of 1.3 hours after 75 or 125 mg MDMA by Vizeli & Liechti. 2017. Journal of Psychopharmacology, 31 (5), 576-588). This avoids the need for re-administration of the entactogen and maintains a consistent plasma concentration, which reduces the unwanted effects associated with nonlinear increases in plasma concentrations.

In some embodiments, 6-MBPB, as a racemic mixture, enantiomerically enriched mixture or pure enantiomer produces less acute cardiovascular effects than MDMA. MDMA produces acute tachycardia and hypertension, which requires safety monitoring and may limit its use in those with preexisting cardiovascular disease (Vizeli & Liechti. 2017. Journal of Psychopharmacology, 31 (5), 576-588; MDMA Investigator's Brochure, 14th Edition: Mar. 18, 2022).

In some embodiments, 6-MBPB has favorable pharmacokinetic properties for administration to a mammal, for example a human. These properties include having more reproducible and less variable pharmacokinetic properties than MDMA. In certain embodiments, 6-MBPB has a less variable maximum plasma concentration (Cmax) than MDMA. In certain embodiments, 6-MBPB has a less variable area-under-the-concentration-versus-time-curve (AUC) than MDMA. An additional potential beneficial property of the present invention is reduced inhibition of CYP enzymes compared to MDMA. Inhibition of such enzymes can cause unwanted toxic drug-drug interactions. In certain embodiments, 6-MBPB does not inhibit or shows minimal inhibition of cytochrome p450 isozyme 2D6 (CYP2D6). In certain embodiments, 6-MBPB shows less potent inhibition of CYP2D6 than MDMA.

The present invention also provides a method for the modulation of CNS activity and/or a method for treatment of mental disorders, including, but not limited to post-traumatic stress and adjustment disorders or any other disorder described herein, comprising administering 6-MBPB or a pharmaceutically acceptable salt or mixture of salts thereof, in an effective amount to a patient such as a human, in a racemic, enantiomerically pure, or enantiomerically enriched form to achieve the desired properties.

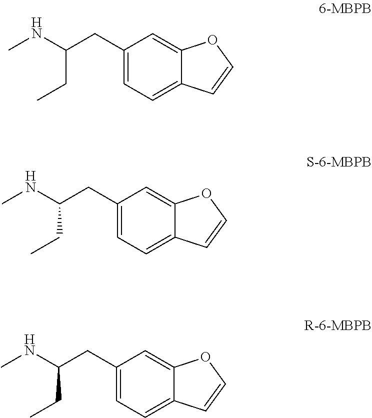

The present invention also provides new medical uses for 6-MBPB, including but not limited to, administration in an effective amount to a host in need thereof such as a human for depression, dysthymia, anxiety, generalized anxiety, social anxiety, panic, adjustment disorders, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism or dissociative disorders or any other disorder described herein, including in the Background.

In other embodiments, 6-MBPB is provided in an effective amount to treat a host, typically a human, with a CNS disorder that can be either a neurological condition (one that is typically treated by a neurologist) or a psychiatric condition (one that is typically treated by a psychiatrist). Neurological disorders are typically those affecting the structure, biochemistry, or normal electrical functions of the brain, spinal cord or other nerves. Psychiatric conditions are more typically thought of as mental disorders, which are primarily abnormalities of thought, feeling or behavior that cause significant distress or impairment of personal functioning.

Thus, 6-MBPB can be used in an effective amount to improve neurological or psychiatric functioning in a patient in need thereof. Neurological indications include, but are not limited to improved neuroplasticity, including treatment of stroke, brain trauma, dementia, and neurodegenerative diseases. MDMA has an EC₅₀ of 7.41 nM for promoting neuritogenesis and an Emax approximately twice that of ketamine, which has fast acting psychiatric benefits that are thought to be mediated by its ability to promote neuroplasticity, including the growth of dendritic spines, increased synthesis of synaptic proteins, and strengthening synaptic responses (Ly et al. Cell reports 23, no. 11 (2018): 3170-3182; Figure S3). 6-MBPB can similarly be considered a psychoplastogen, that is, small molecules that are able to induce rapid neuroplasticity (Olson, 2018, Journal of experimental neuroscience, 12, 1179069518800508. https://doi.org/10.1177% 2F1179069518800508). For example, in certain embodiments, the disclosed compounds and compositions can be used to improve stuttering and other dyspraxias or to treat Parkinson's disease or schizophrenia.

In other embodiments, 6-MBPB may be used in an effective amount to treat a host, typically a human, to modulate an immune or inflammatory response. The compounds disclosed herein alter extracellular serotonin, which is known to alter immune functioning. MDMA produces acute time-dependent increases and decreases in immune response.

In certain embodiments, 6-MBPB is used as described herein in enantiomerically enriched form to achieve the goals of the invention. In other embodiments, the compound is used as a racemate or a pure, including a substantially pure enantiomer.

The invention additionally includes methods to treat a neurological or psychiatric central nervous system disorder as further described herein, including a mental disorder, or to provide a mental enhancement, with 6-MBPB or a pharmaceutically acceptable salt or mixed salt thereof.

In further embodiments, the invention includes methods to treat a neurological or psychiatric central nervous system disorder as further described herein with enantiomerically enriched 6-MBPB.

In certain aspects of these embodiments, 6-MBPB can be improved or “tuned” by administering an effective amount to a host such as a human, in need thereof, in a composition of an enantiomerically enriched composition that has an abundance of one enantiomer over the other or a substantially pure enantiomer (or diastereomer, where relevant), or a mixture thereof. As described above, the enantiomers act differently from each other on various receptors and transporters, producing variable effects, and that those effects can be selected for based on desired outcome for the patient.

In certain embodiments, 6-MBPB or mixtures of the present invention are administered to a human patient in an effective amount in conjunction with psychotherapy, cognitive enhancement, or life coaching (pharmacotherapy), or as part of routine medical therapy.

6-MBPB, including the enantiomerically enriched 6-MBPB, can be used in the form of a pharmaceutically acceptable salt or a mixture of salts. Nonlimiting examples include those wherein the pharmaceutically acceptable salt(s) is selected from HCl, sulfate, aspartate, saccharate, phosphate, oxalate, acetate, amino acid anion, gluconate, maleate, malate, citrate, mesylate, nitrate or tartrate, or a mixture thereof.

### 6-MBPB Properties

6-MBPB has beneficial selectivity profiles for neurotransmitter transporters compared to MDMA or other known entactogens. 6-MBPB is a 5-HT releaser and is only partial releaser of norepinephrine. Additionally, 6-MBPB enantiomers have differing effects on dopamine. While S-6-MBPB is a partial releaser of dopamine, R-6-MBPB is a dopamine uptake inhibitor. The inhibition of dopamine uptake creates a concentration-dependent ceiling on dopamine release that will lower the DAT to SERT ratio of compound at higher concentrations. This surprising combination is highly desirable. The balance of weakly activating NET (to reduce cardiovascular toxicity risk) and decreasing the DAT to SERT ratio over the racemate (to increase therapeutic effect relative to addictive liability) with higher doses is a desirable feature of an entactogenic therapy displayed by the compounds and compositions of the present invention.

In some embodiments, 6-MBPB, as a racemic mixture, enantiomerically enriched mixture or pure enantiomer surprisingly lacks interactions with hTAAR1 (human trace amine-associated receptor), even at concentrations up to 60 μM. TAAR1 is an intracellular receptor expressed within the presynaptic terminal of monoamine neurons. MDMA and non-cathinone stimulants bind to and stimulate TAAR1 (e.g., Rickli, et al, 2015. British journal of pharmacology, 172 (13), pp. 3412-3425.), which decreases and shortens the acute therapeutic effects of these drugs, at least in part by causing internalization of the monoamine transporter (Underhill, & Amara. 2020. The FASEB Journal, 34 (S1), pp. 1-1). This internalization of monoamine transporter contributes to short-term tolerance (tachyphylaxis) to the entactogen and may further contribute to decreased mood and undesirable effects in the week after drug exposure. It is both advantageous and unexpected for an entactogen to lack effects at TAAR1.

The present invention also provides a method for the modulation of CNS activity and/or a method for treatment of mental disorders, including, but not limited to post-traumatic stress and adjustment disorders or any other disorder described herein, comprising administering 6-MBPB or a pharmaceutically acceptable salt or mixture of salts thereof, in an effective amount to a patient such as a human, in a racemic, enantiomerically pure, or enantiomerically enriched form to achieve the desired properties.

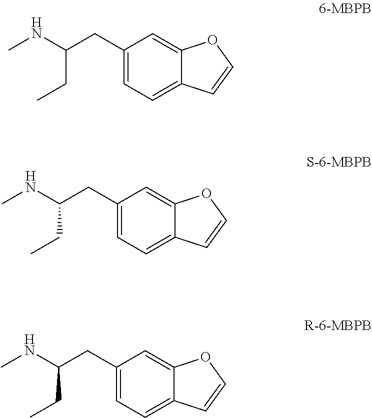

The present invention also provides new medical uses for 6-MBPB, including but not limited to, administration in an effective amount to a host in need thereof such as a human for depression, dysthymia, anxiety, generalized anxiety, social anxiety, panic, adjustment disorders, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism or dissociative disorders or any other disorder described herein, including in the Background.

In other embodiments, 6-MBPB may be used in an effective amount to treat a host, typically a human, to modulate an immune or inflammatory response. The compounds disclosed herein alter extracellular serotonin, which is known to alter immune functioning. MDMA produces acute time-dependent increases and decreases in immune response.

In certain embodiments, 6-MBPB is used as described herein in enantiomerically enriched form to achieve the goals of the invention. In other embodiments, the compound is used as a racemate or a pure, including a substantially pure enantiomer.

The invention additionally includes methods to treat a neurological or psychiatric central nervous system disorder as further described herein, including a mental disorder, or to provide a mental enhancement, with 6-MBPB or a pharmaceutically acceptable salt or mixed salt thereof.

In further embodiments, the invention includes methods to treat a neurological or psychiatric central nervous system disorder as further described herein with enantiomerically enriched 6-MBPB.

The PCT Application entitled “ADVANTAGEOUS BENZOFURAN COMPOSITIONS FOR MENTAL DISORDERS OR ENHANCEMENT,” filed on Jun. 8, 2021 as International Application No. PCT/US2021/036479, and each of the U.S. Provisional Applications from which it draws priority (see id. at lines 5-11, Cross-Reference to Related Applications), are incorporated by reference for all purposes as if fully set forth herein.

### Enantiomers

6-MBPB is an entactogen with unexpected properties that improve upon MDMA and known compounds. 6-MBPB may be in a racemic form, as a pure enantiomer, or as an enantiomerically enriched mixture, or a pharmaceutically acceptable salt or mixed salts thereof.

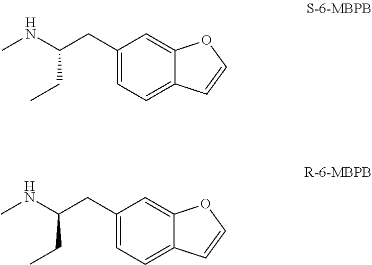

In certain embodiments the 6-MBPB compound is an enantiomerically enriched mixture for example an S-enantiomer contains at least 55% of the S-enantiomer, and, typically at least about 60%, 65%, 70%, 75%, 80%, 85%, 90%, or 95% or more of the S-enantiomer or for example an R-enantiomer contains at least 55% of the R-enantiomer, and typically at least about 60%, 65%, 70%, 75%, 80%, 85%, 90% or 95% of the R-enantiomer. The specific ratio of S or R enantiomer can be selected for the need of the patient according to the health care specialist to balance the desired effect.

Particular embodiments for pharmaceutical compositions with one or more active agents, including enantiomerically enriched pharmaceutical compositions, of the present invention include:
a) RS-6-MBPB
b) S-6-MBPB;
c) R-6-MBPB;
d) A mixture of S-6-MBPB, R-6-MBPB and there is more S-enantiomer than R-enantiomer;
e) A mixture of S-6-MBPB, R-6-MBPB and there is less S-enantiomer than R-enantiomer;
f) A mixture of S-6-MBPB, R-6-MBPB and about 65% is the S-enantiomer while about 35% is the R-enantiomer;
g) A mixture of S-6-MBPB, R-6-MBPB and greater than about 65% is the S-enantiomer while less than about 35% is the R-enantiomer;
h) A mixture of S-6-MBPB, R-6-MBPB and greater than about 90% is the S-enantiomer while less than about 10% is the R-enantiomer;
i) A mixture of S-6-MBPB, R-6-MBPB and about 35% is the S-enantiomer while about 65% is the R-enantiomer;
j) A mixture of S-6-MBPB, R-6-MBPB and less than about 35% is the S-enantiomer while greater than about 65% is the R-enantiomer;
k) A mixture of S-6-MBPB, R-6-MBPB and less than about 10% is the S-enantiomer while greater than about 90% is the R-enantiomer;
It will be understood that the above embodiments and classes of embodiments can be combined to form additional preferred embodiments.
   Methods to Treat CNS Disorders Including Mental Disorders and for Mental Enhancement with 6-MBPB

The present invention provides methods and uses for the treatment of CNS disorders, including, but not limited to, mental disorders as described herein, including post-traumatic stress and adjustment disorders, comprising administering 6-MBPB or a composition or a pharmaceutically acceptable salt or mixture of salts thereof as described herein. It has been surprisingly discovered that these compounds display many pharmacological properties that are beneficial to their use as therapeutics and represent an improvement over existing therapeutics.

The methods described herein can utilize 6-MBPB by itself, as an enantiomerically enriched mixture, or as a composition or pharmaceutically acceptable salt thereof.

The present invention also provides, for example, methods of using 6-MBPB for the treatment of disorders, including, but not limited to depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making.

In other embodiments, 6-MBPB is provided in an effective amount to treat a host, typically a human, with a CNS disorder that can be either a neurological condition (one that is typically treated by a neurologist) or a psychiatric condition (one that is typically treated by a psychiatrist). Neurological disorders are typically those affecting the structure, biochemistry or cause electrical abnormalities of the brain, spinal cord or other nerves. Psychiatric conditions are more typically thought of as mental disorders, which are primarily abnormalities of thought, feeling or behavior that cause significant distress or impairment of personal functioning.

Thus, 6-MBPB can be used in an effective amount to improve neurological or psychiatric functioning in a patient in need thereof. Neurological indications include, but are not limited to improved neuroplasticity, including treatment of stroke, brain trauma, dementia, and neurodegenerative diseases. MDMA has been reported to have an EC50 of 7.41 nM for promoting neuritogenesis and an Emax approximately twice that of ketamine, which has fast acting psychiatric benefits that are thought to be mediated by its ability to promote neuroplasticity, including the growth of dendritic spines, increased synthesis of synaptic proteins, and strengthening synaptic responses. Figure S3. in Ly et al. (Cell reports 23, no. 11 (2018): 3170-3182, https://doi.org/10.1016/j.celrep.2018.05.022). The compounds of the current invention can similarly be considered psychoplastogens, that is, small molecules that are able to induce rapid neuroplasticity (Olson, 2018, Journal of experimental neuroscience, 12, 1179069518800508. https://doi.org/10.1177% 2F1179069518800508). For example, in certain embodiments, the disclosed compounds and compositions can be used to improve stuttering and other dyspraxias or to treat Parkinson's disease or schizophrenia.

In other embodiments, the 6-MBPB may be used in an effective amount to treat a host, typically a human, to modulate an immune or inflammatory response. The compounds disclosed herein alter extracellular serotonin, which is known to alter immune functioning. MDMA produces acute time-dependent increases and decreases in immune response.

The following nonlimiting examples are relevant to any of the disorders, indications, methods of use or dosing regimes described herein.

In certain embodiments, a host, for example a human, is treated with an effective amount of 6-MBPB in a racemic or enantiomerically pure composition,

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 99 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 95 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 90 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 85 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 80 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 75 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 70 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 65 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 60 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 55 percent.

In certain embodiments, a host, for example a human, is treated with an effective amount of 6-MBPB in or an enantiomerically enriched mixture, or a pharmaceutically acceptable salt or mixed salt, isotopic derivative, or prodrug thereof, wherein the percent of S enantiomer is greater than about 55 or 60 percent.

In one embodiment, a method is provided to treat diseases or disorders linked to inadequate functioning of neurotransmission in the CNS comprising administering 6-MBPB or a pharmaceutically acceptable salt thereof in a host in need thereof.

This invention also provides the administration of an effective amount of 6-MBPB or a pharmaceutically acceptable salt or composition to a host, typically a human, to treat a maladaptive response to perceived psychological threats. In one embodiment, 6-MBPB or a pharmaceutically acceptable salt or composition is administered in the context of psychotherapy. In one embodiment, 6-MBPB or a pharmaceutically acceptable salt or composition is administered as a stand-alone treatment.

Non-Limiting Examples of Pharmacotherapeutic Use Psychotherapy, cognitive enhancement, or life coaching conducted with 6-MBPB as an adjunct (hereafter, “pharmacotherapy”) is typically conducted in widely spaced sessions with one, two, or rarely three or more administrations of an entactogen per session. These sessions can be as frequent as weekly, but are more often approximately monthly or even less frequently. In most cases, a small number of pharmacotherapy sessions, on the order of one to three, is needed for the patient to experience significant clinical progress, as indicated, for example, by a reduction in signs and symptoms of mental distress, by improvement in functioning in some domain of life, by arrival at a satisfactory solution to some problem, or by increased feelings of closeness to and understanding of some other person. In some embodiments, the psychotherapy, cognitive enhancement, or life coaching is conducted with RS-6-MBPB, S-6-MBPB, R-6-MBPB, or an effective amount of enantiomerically enriched 6-MBPB or a pharmaceutically acceptable salt thereof.

The following sections provide detailed examples of pharmacotherapy. While common procedures are described, these are intended as illustrative, non-limiting examples. It is anticipated that the prescribing physician and therapy team may wish to specify different procedures than those described here based on their clinical judgment concerning the needs of the patient.

The example methods of treatment can also be modified with very minor changes to treat multiple patients at once, including couples or families. Hence, “patient” should be understood to mean one or more individuals.

Use of 6-MBPB in Conjunction with Conventional Psychotherapy or Coaching

In one embodiment, 6-MBPB is integrated into the patient's ongoing psychotherapy or coaching (hereafter abbreviated as “psychotherapy”). If a patient in need of the pharmacotherapy is not in ongoing psychotherapy, then psychotherapy may be initiated and the pharmacotherapy added later, after the prescribing physician and treating psychotherapist, physician, coach, member of the clergy, or other similar professional or someone acting under the supervision of such a professional (hereafter, “therapist”) agree that the pharmacotherapy is indicated and that there have been sufficient meetings between the patient and therapist to establish an effective therapeutic alliance.

If the patient is not experienced with the pharmacotherapy, a conversation typically occurs in which the therapist or other members of the therapy team addresses the patient's questions and concerns about the medicine and familiarizes the patient with the logistics of pharmacotherapy-assisted session. The therapist describes the kinds of experience that can be expected during the pharmacotherapy session. Optionally, parts of this conversation employ written, recorded, or interactive digital explanations, as might be used in the informed consent process in a clinical trial.

The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

The compounds and compositions of the invention (or alternately herein for convenience, the “medicine”) is administered shortly before or during a scheduled psychotherapy session, with timing optionally selected so that therapeutic effects begin by the time the psychotherapy session begins. Either shortly before or after administration of the medicine, it is common for the therapist to provide some reminder of their mutual commitments and expected events during the session.

The psychotherapy session is carried out by the therapist, who, optionally, may be remote and in communication with the patient using a communication means suitable for telehealth or telemedicine, such as a phone, video, or other remote two-way communication method. Optionally, video or other monitoring of the patient's response or behavior is used to document or measure the session. The therapist uses their clinical judgment and available data to adjust the session to the needs of the patient. Many therapists view their responsibility as being to facilitate rather than direct the patient's experience. This may sometimes involve silent empathic listening, while other times it may include more active support to help the patient arrive at new perspectives on their life.

It is anticipated that the therapeutic effects of the medicine will allow the patient to make more rapid therapeutic progress than would normally be possible. These effects include decreased neuroticism and increased feelings of authenticity. Patients are often able to calmly contemplate actual or possible experiences that would normally be upsetting or even overwhelming. This can facilitate decision making and creativity in addition to mental wellness.

Optionally, the prescribing physician may allow a second or even third administration of the medicine or another psychotherapeutic agent in order to extend the therapeutic effects. Optionally, a pharmaceutical preparation with modified release is employed to make this unnecessary.

Because the duration of the scheduled psychotherapy session may be shorter than the therapeutic effects of the medicine, the therapist may suggest to the patient activities to support further psychotherapeutic progress after the psychotherapy session has ended. Alternatively, the therapist may continue to work with the patient until the therapeutic effects of the medicine have become clinically minimal.

In a subsequent non-pharmacological psychotherapy session, the therapist and patient will typically discuss the patient's experiences from the pharmacotherapy session and the therapist will often aid the patient in recalling the therapeutic effects and help them to incorporate the experiences into their everyday lives.

Pharmacotherapy sessions may be repeated as needed, based on the judgment of the treating physician and therapy team regarding the needs of the patient.

### Use of 6-MBPB Outside of Conventional Psychotherapy

In one embodiment, 6-MBPB (“the medicine” as used in this section) is administered outside of a conventional psychotherapy. This example method is a broader, more flexible approach to pharmacotherapy that is not centered on supervision by a therapist. These pharmacotherapy sessions can take place in many different quiet and safe settings, including the patient's home. The setting is typically chosen to offer a quiet setting, with minimal disruptions, where the patient feels psychologically safe and emotionally relaxed. The setting may be the patient's home but may alternatively be a clinic, retreat center, or hotel room.

In one alternative embodiment, the medicine is taken by the patient regularly to maintain therapeutic concentrations of the active compound in the blood. In another alternative embodiment, the medicine is taken, as needed, for defined psychotherapy sessions.

Optionally, a checklist may be followed to prepare the immediate environment to minimize distractions and maximize therapeutic or decision-making benefits. This checklist can include items such as silencing phones and other communications devices, cleaning and tidying the environment, preparing light refreshments, preparing playlists of appropriate music, and pre-arranging end-of-session transportation if the patient is not undergoing pharmacotherapy at home.

Before the pharmacotherapy session, there may be an initial determination of the therapeutic or other life-related goals (for example, decision-making, increasing creativity, or simply appreciation of life) that will be a focus of the session. These goals can optionally be determined in advance with support from a therapist.

Optionally, the therapist may help the patient select stimuli, such as photographs, videos, augmented or virtual reality scenes, or small objects such as personal possessions, that will help focus the patient's attention on the goals of the session or on the patient's broader life journey. As examples that are intended to be illustrative and not restrictive, these stimuli can include photographs of the patient from when they were young, which can increase self-compassion, or can include stimuli relating to traumatic events or phobias experienced by the patient, which can help the patient reevaluate and change their response to such stimuli. Optionally, the patient selects these stimuli without assistance (e.g., without the involvement of the therapist) or does not employ any stimuli. Optionally, stimuli are selected in real time by the therapist or an algorithm based on the events of the session with the goal of maximizing benefits to the patient.

If the patient is not experienced with the pharmacotherapy, a conversation occurs in which the therapist addresses the patient's questions and concerns about the medicine and familiarizes the patient with the logistics of a pharmacotherapy-assisted session. The therapist describes the kinds of experience that can be expected during the pharmacotherapy-assisted session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

Selected session goals and any commitments or other agreements regarding conduct between the patient and therapy team are reviewed immediately before administration of the medicine. Depending on the pharmaceutical preparation and route of administration, the therapeutic effects of the medicine usually begin within one hour. Typical therapeutic effects include decreased neuroticism and increased feelings of authenticity. Patients are often able to calmly contemplate experiences or possible experiences that would normally be upsetting or even overwhelming. This can facilitate decision making and creativity in addition to mental wellness.

Optionally, sleep shades and earphones with music or soothing noise may be used to reduce distractions from the environment. Optionally, a virtual reality or immersive reality system may be used to provide stimuli that support the therapeutic process. Optionally, these stimuli are preselected; optionally, they are selected in real time by a person or an algorithm based on events in the session with the goal of maximizing benefits to the patient. Optionally, a therapist or other person well-known to the patient is present or available nearby or via phone, video, or other communication method in case the patient wishes to talk, however the patient may optionally undergo a session without the assistance of a therapist. Optionally, the patient may write or create artwork relevant to the selected session goals. Optionally, the patient may practice stretches or other beneficial body movements, such as yoga (“movement activity”).

Optionally, in other embodiments the patient may practice movement activity that includes more vigorous body movements, such as dance or other aerobic activity. Movement activity also may make use of exercise equipment such as a treadmill or bicycle.

In some additional embodiments, the patient may be presented with music, video, auditory messages, or other perceptual stimuli. Optionally, these stimuli may be adjusted based on the movements or other measurable aspects of the patient. Such adjustment may be done by the therapist with or without the aid of a computer, or by a computer alone in response to said patient aspects, including by an algorithm or artificial intelligence, and “computer” broadly meaning any electronic tool suitable for such purposes, whether worn or attached to a patient (e.g., watches, fitness trackers, “wearables,” and other personal devices; biosensors or medical sensors; medical devices), whether directly coupled or wired to a patient or wirelessly connected (and including desktop, laptop, and notebook computers; tablets, smartphones, and other mobile devices; and the like), and whether within the therapy room or remote (e.g., cloud-based systems).

For example, measurable aspects of a patient (e.g., facial expression, eye movements, respiration rate, pulse rate, skin color change, patient voice quality or content, patient responses to questions) from these tools may be individually transformed into scores on standardized scales by subtracting a typical value and then multiplying by a constant and these scores may be further multiplied by constants and added together to create an overall score that can optionally be transformed by multiplication with a link function, such as the logit function, to create an overall score. This score may be used to select or adjust stimuli such as selecting music with higher or lower beats-per-minute or with faster or slower notes, selecting images, audio, or videos with different emotionality or autobiographical meaning, or selecting activities for the patient to engage in (such as specific movements, journaling prompts, or meditation mantras).

It should be readily appreciated that a patient can participate in numerous therapeutically beneficial activities, where such participation follows or is in conjunction with the administration of a compound or composition of the invention, including writing about a preselected topic, engaging in yoga or other movement activity, meditating, creating art, viewing of photographs or videos or emotionally evocative objects, using a virtual reality or augmented reality system, talking with a person, and thinking about a preselected problem or topic, and it should be understood that such participation can occur with or without the participation or guidance of a therapist.

Optionally, the prescribing physician may allow a second or even third administration of the medicine or another psychotherapeutic agent in order to extend the therapeutic effects. Optionally, a pharmaceutical preparation with modified release is employed to make this unnecessary.

The patient typically remains in the immediate environment until the acute therapeutic effects of the medicine are clinically minimal, usually within eight hours. After this point, the session is considered finished.

The treatment plan will often include a follow-up session with a therapist. This follow-up session occurs after the pharmacotherapy session has ended, often the next day but sometimes several days later. In this session, the patient discusses their experiences from the pharmacotherapy session with the therapist, who can aid them in recalling the therapeutic effects and help them to incorporate the experiences into their everyday lives.

Pharmacotherapy sessions may be repeated as needed, based on the judgment of the treating physician and therapy team regarding the needs of the patient.

### Pharmaceutical Compositions Comprising 6-MBPB

The 6-MBPB can be administered in an effective amount as the neat chemical but are more typically administered as a pharmaceutical composition for a host, typically a human, in need of such treatment in an effective amount for any of the disorders described herein. The compounds or compositions disclosed herein may be administered orally, topically, systemically, parenterally, by inhalation, insufflation, or spray, mucosally (e.g., buccal, sublingual), sublingually, transdermally, rectally, intravenous, intra-aortal, intracranial, subdermal, intraperitoneal, intramuscularly, inhaled, intranasal, subcutaneous, transnasal, or by other means, in dosage unit formulations containing conventional pharmaceutically acceptable carriers. Such compositions are prepared in a manner well known in the pharmaceutical art and comprise at least one active compound. (See, e.g., Remington, 2005, Remington: The science and practice of pharmacy, 21st ed., Lippincott Williams & Wilkins.)

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, a suppository, a buccal or sublingual formulation, a parenteral formulation, an ophthalmic solution, or in a medical device. Some dosage forms, such as tablets and capsules, are subdivided into suitably sized unit doses containing appropriate quantities of the active components, e.g., an effective amount to achieve the desired purpose.

A “pharmaceutically acceptable composition” thus refers to at least one compound (which may be a mixture of enantiomers or diastereomers, as fully described herein) of the invention and a pharmaceutically acceptable vehicle, excipient, diluent or other carrier in an effective amount to treat a host, typically a human, who may be a patient.

In certain nonlimiting embodiments the pharmaceutical composition is a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 20, 25, 40, 50, 100, 125, 150, 200, 250, 300, 400, 500, 600, 700, or 750 mg of active compound, or its salt or mixed salt.

The pharmaceutical compositions described herein can be formulated into any suitable dosage form, including tablets, capsules, gelcaps, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, pills, powders, delayed-release formulations, immediate-release formulations, modified release formulations, extended-release formulations, pulsatile release formulations, multi particulate formulations, and mixed immediate release and controlled release formulations. Generally speaking, the composition should be administered in an effective amount to administer an amount of the active agents of the present invention achieves a plasma level commensurate with the concentrations found to be effective in vivo for a period of time effective to elicit a desired therapeutic effect without abuse liability.

In making the compositions employed in the present invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, solutions, slurries, syrups, aerosols (as a solid or in a liquid medium), ointments containing for example up to 10% by weight of the active compound, soft and hard gelatin capsules, suppositories, dry powders for inhalation, liquid preparations for vaporization and inhalation, topical preparations, transdermal patches, sterile injectable solutions, and sterile packaged powders. Compositions may be formulated as immediate release, controlled release, sustained (extended) release or modified release formulations.

The compositions of the present invention can be administered by multiple routes, which may differ in different patients according to their preference, co-morbidities, side effect profile, and other factors (IV, PO, transdermal, etc.). In one embodiment, the pharmaceutical composition includes the presence of other substances with the active drugs, known to those skilled in the art, such as fillers, carriers, gels, skin patches, lozenges, or other modifications in the preparation to facilitate absorption through various routes (such as, but not limited to, gastrointestinal, transdermal, etc.) and/or to extend the effect of the drugs, and/or to attain higher or more stable serum levels or to enhance the therapeutic effect of the active drugs in the combination.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, but are not limited to, lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from at least about 0.05 to about 350 mg or less, more preferably at least about 5.0 to about 180 mg or less, of the active ingredients. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent, or excipient.

The active compounds are effective over a wide dosage range. For example, as-needed dosages normally fall within the range of at least about 0.01 to about 4 mg/kg or less. In the treatment of adult humans, the range of at least about 0.2 to about 3 mg/kg or less, in single dose, is especially preferred.

It will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way.

In some instances, dosage levels below the lower limit of the aforesaid range may be more than adequate, while in other cases still larger doses may be employed without causing any harmful side effects, provided for instance that such larger doses may be first divided into several smaller doses for administration.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

In one embodiment, a powder comprising the active agents of the present invention described herein may be formulated to comprise one or more pharmaceutical excipients and flavors. Such a powder may be prepared, for example, by mixing the active agents of the present invention and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

### Oral Formulations

In certain embodiments, 6-MBPB is formulated in an effective amount in an pharmaceutically acceptable oral dosage form. Oral dosage forms may include, but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. The oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agent of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

Alternatively, the pharmaceutical solid dosage forms described herein can comprise the active agent or agents of the present invention (i.e., the “active agent(s)”; but for convenience herein, both “active agent” and “active agents” shall mean “active agent(s)” unless context clearly indicates that what is intended or would be suitable is only one agent or only two or more agents) and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the active agent of the present invention formulation. In one embodiment, some or all of the active agent of the present invention particles are coated. In another embodiment, some or all of the active agent of the present invention particles are microencapsulated. In yet another embodiment, some or all of the active agent of the present invention is amorphous material coated and/or microencapsulated with inert excipients. In still another embodiment, the active agent of the present invention particles are not microencapsulated and are uncoated.

Suitable carriers for use in the solid dosage forms described herein include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft- or hard-shell capsules and in tablet formulation, binders ensure that the tablet remains intact after compression and help assure blend uniformity prior to a compression or fill step. Materials suitable for use as binders in the solid dosage forms described herein include carboxymethylcellulose, methylcellulose (e.g., Methocel®), hydroxypropylmethylcellulose (e.g., Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are typically used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm3, e.g. Avicel®, powdered cellulose), and talc.

Suitable wetting agents for use in the solid dosage forms described herein include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in the solid dosage forms described here include polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, e.g., gum tragacanth and gum acacia, guar gum, xanthans, including xanthan gum, sugars, cellulosic, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, e.g., butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining superdisintegrants such as Croscarmellose sodium and different grades of microcrystalline cellulose in different ratios. To aid disintegration, sodium starch glycolate will be added.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as ethanol, isopropyl alcohol, hexadecyl alcohol, glycerol, and propylene glycol. Ethers, such as poly(ethylene glycol), petroleum hydrocarbons such as mineral oil and petrolatum, and water may also be used in suspension formulations.

In some embodiments, formulations are provided comprising particles of 6-MBPB and at least one dispersing agent or suspending agent for oral administration to a subject in need thereof.

The formulation may be a powder and/or granules for suspension, and upon admixture with water, a substantially uniform suspension is obtained. As described herein, the aqueous dispersion can comprise amorphous and non-amorphous particles consisting of multiple effective particle sizes such that the drug is absorbed in a controlled manner over time. In certain embodiments, the aqueous dispersion or suspension is an immediate-release formulation. In another embodiment, an aqueous dispersion comprising amorphous particles is formulated such that a portion of the particles of the present invention are absorbed within, e.g., about 0.75 hours after administration and the remaining particles are absorbed 2 to 4 hours after absorption of the earlier particles.

In other embodiments, addition of a complexing agent to the aqueous dispersion results in a larger span of the particles to extend the drug absorption phase of the active agent such that 50-80% of the particles are absorbed in the first hour and about 90% are absorbed by about 4 hours. Dosage forms for oral administration can be aqueous suspensions selected from the group including pharmaceutically acceptable aqueous oral dispersions, emulsions, solutions, and syrups. See, e.g., Singh et al., Encyclopedia of Pharm. Tech., 2nd Ed., 754-757 (2002). In addition to the active agents of the present invention particles, the liquid dosage forms may comprise additives, such as (a) disintegrating agents; (b) dispersing agents; (c) wetting agents; (d) at least one preservative; (e) viscosity enhancing agents; (f) at least one sweetening agent; and (g) at least one flavoring agent.

Examples of disintegrating agents for use in the aqueous suspensions and dispersions include a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corp., Parsippany, N.J.)).

In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; noncrystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908® or Poloxamine 908®).

Wetting agents (including surfactants) suitable for the aqueous suspensions and dispersions described herein are known in the art and include acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one embodiment, the aqueous liquid dispersion can comprise methylparaben and propylparaben in a concentration ranging from at least about 0.01% to about 0.3% or less methylparaben by weight to the weight of the aqueous dispersion and at least about 0.005% to about 0.03% or less propylparaben by weight to the total aqueous dispersion weight. In yet another embodiment, the aqueous liquid dispersion can comprise methylparaben from at least about 0.05 to about 0.1 or less weight % and propylparaben from at least about 0.01 to about 0.02 or less weight % of the aqueous dispersion.

Suitable viscosity enhancing agents for the aqueous suspensions or dispersions described herein include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid formulations of the present invention can also comprise inert diluents commonly used in the art, such as water or other solvents, solubilizing agents, emulsifiers, and/or sweeteners.

In one embodiment, the formulation for oral delivery is an effervescent powder containing 6-MBPB or a pharmaceutically acceptable salt thereof. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

Tablets of the invention described here can be prepared by methods well known in the art. Various methods for the preparation of the immediate release, modified release, controlled release, and extended-release dosage forms (e.g., as matrix tablets, tablets having one or more modified, controlled, or extended-release layers, etc.) and the vehicles therein are well known in the art. Generally recognized compendia of methods include: Remington: The Science and Practice of Pharmacy, Alfonso R. Gennaro, Editor, 20th Edition, Lippincott Williams & Wilkins, Philadelphia, PA; and Sheth et al. (1980), Compressed tablets, in Pharmaceutical dosage forms, Vol. 1, edited by Lieberman and Lachtman, Dekker, NY.

In certain embodiments, solid dosage forms, e.g., tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include, e.g., spray drying, pan coating, melt granulation, granulation, fluidized bed spray drying or coating (e.g., Wurster coating), tangential coating, top spraying, tableting, extruding and the like.

Compressed tablets are solid dosage forms prepared by compacting the bulk blend the active agents of the present invention formulations described above. In various embodiments, compressed tablets which are designed to dissolve in the mouth will comprise one or more flavoring agents. In other embodiments, the compressed tablets will comprise a film surrounding a final compressed tablet. In some embodiments, the film coating can provide a delayed release of the active agents of the present invention formulation. In other embodiments, the film coating aids in patient compliance (e.g., Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, e.g., by placing the bulk blend of the active agents of the present invention formulation, described above, inside of a capsule. In some embodiments, the formulations of the present invention (non-aqueous suspensions and solutions) are placed in a soft gelatin capsule. In other embodiments, the formulations of the present invention are placed in standard gelatin capsules or non-gelatin capsules such as capsules comprising HPMC. In other embodiments, the formulations of the present invention are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the active agents of the present invention is delivered in a capsule form.

In certain embodiments, ingredients (including or not including the active agent) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, e.g., via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include U.S. Pat. Nos. 4,343,789; 5,340,591; 5,456,923; 5,700,485; 5, 723,269; and U.S. Pub. No. 2004/0013734. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet.

### Non-Limiting Examples of Formulations for Oral Delivery

In one non-limiting embodiment, hard gelatin capsules comprising the following ingredients are prepared by mixing the ingredients and filling into hard gelatin capsules in 340 mg quantities.

| Ingredient | Quantity (mg/capsule) |
|---|---|
| S-6-MBPB | 30.0 |
| Starch | 205.0 |
| Alpha lipoic acid | 100.0 |
| Magnesium stearate | 5.0 |

In one non-limiting embodiment, hard gelatin capsules comprising the following ingredients are prepared by mixing the ingredients and filling into hard gelatin capsules in 340 mg quantities.

| Ingredient | Quantity (mg/capsule) |
|---|---|
| 6-MBPB (100% R-enantiomer) | 30.0 |
| Starch | 205.0 |
| Alpha lipoic acid | 100.0 |
| Magnesium stearate | 5.0 |

In one non-limiting embodiment, hard gelatin capsules comprising the following ingredients are prepared by mixing the ingredients and filling into hard gelatin capsules in 340 mg quantities.

| Ingredient | Quantity (mg/capsule) |
|---|---|
| RS-6-MBPB | 30.0 |
| Starch | 205.0 |
| Alpha lipoic acid | 100.0 |
| Magnesium stearate | 5.0 |

In one non-limiting embodiment, hard gelatin capsules comprising the following ingredients are prepared by mixing the ingredients and filling into hard gelatin capsules in 340 mg quantities.

| Ingredient | Quantity (mg/capsule) |
|---|---|
| RS-6-MBPB | 30.0 |
| Starch | 205.0 |
| Alpha lipoic acid | 100.0 |
| Magnesium stearate | 5.0 |

In one non-limiting embodiment, hard gelatin capsules comprising the following ingredients are prepared by mixing the ingredients and filling into hard gelatin capsules in 340 mg quantities.

| Ingredient | Quantity (mg/capsule) |
|---|---|
| 6-MBPB (70% S-enantiomer) | 30.0 |
| Starch | 205.0 |
| Alpha lipoic acid | 100.0 |
| Magnesium stearate | 5.0 |

In one non-limiting embodiment, hard gelatin capsules comprising the following ingredients are prepared by mixing the ingredients and filling into hard gelatin capsules in 340 mg quantities.

| Ingredient | Quantity (mg/capsule) |
|---|---|
| 6-MBPB (70% S-enantiomer) | 30.0 |
| Starch | 205.0 |
| Alpha lipoic acid | 100.0 |
| Magnesium stearate | 5.0 |

In one non-limiting embodiment, a tablet formulation is prepared comprising the ingredients below. The components are blended and compressed to form tablets, each weighing 240 mg.

| Ingredient | Quantity (mg/tablet) |
|---|---|
| R-6-MBPB | 25.0 |
| Cellulose, microcrystalline | 200.0 |
| Colloidal silicon dioxide | 10.0 |
| Stearic acid | 5.0 |

In one non-limiting embodiment, a tablet formulation is prepared comprising the ingredients below. The components are blended and compressed to form tablets, each weighing 240 mg.

| Ingredient | Quantity (mg/tablet) |
|---|---|
| 6-MBPB (70% R-enantiomer) | 25.0 |
| Cellulose, microcrystalline | 200.0 |
| Colloidal silicon dioxide | 10.0 |
| Stearic acid | 5.0 |

In one non-limiting embodiment, a tablet formulation is prepared comprising the ingredients below. The components are blended and compressed to form tablets, each weighing 240 mg.

| Ingredient | Quantity (mg/tablet) |
|---|---|
| 6-MBPB (60% S-enantiomer) | 25.0 |
| Cellulose, microcrystalline | 200.0 |
| Colloidal silicon dioxide | 10.0 |
| Stearic acid | 5.0 |

In one non-limiting embodiment, a tablet formulation is prepared comprising the ingredients below. The components are blended and compressed to form tablets, each weighing 240 mg.

| Ingredient | Quantity (mg/tablet) |
|---|---|
| 6-MBPB (70% S-enantiomer) | 25.0 |
| Cellulose, microcrystalline | 200.0 |
| Colloidal silicon dioxide | 10.0 |
| Stearic acid | 5.0 |

In one non-limiting embodiment, a tablet formulation is prepared comprising the ingredients below. The components are blended and compressed to form tablets, each weighing 240 mg.

| Ingredient | Quantity (mg/tablet) |
|---|---|
| 6-MBPB (70% S-enantiomer) | 25.0 |
| Cellulose, microcrystalline | 200.0 |
| Colloidal silicon dioxide | 10.0 |
| Stearic acid | 5.0 |

In one non-limiting embodiment, a tablet formulation is prepared comprising the ingredients below. The components are blended and compressed to form tablets, each weighing 240 mg.

| Ingredient | Quantity (mg/tablet) |
|---|---|
| RS-6-MBPB | 25.0 |
| Cellulose, microcrystalline | 200.0 |
| Colloidal silicon dioxide | 10.0 |
| Stearic acid | 5.0 |

In one non-limiting embodiment, a tablet, comprising the components below, including R-6-MBPB and S-6-MBPB, is prepared. The active ingredients, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

| Ingredient | Quantity (mg/tablet) |
|---|---|
| R-6-MBPB | 20.0 |
| S-6-MBPB | 10.0 |
| Starch | 45.0 |
| Microcrystalline cellulose | 35.0 |
| Polyvinylpyrrolidone (as 10% solution in water) | 4.0 |
| Sodium carboxymethyl starch | 4.5 |
| Magnesium stearate | 0.5 |
| Talc | 1.0 |

In one non-limiting embodiment, a tablet, comprising the components below, including R-6-MBPB and MDMA, is prepared. The active ingredients, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

| Ingredient | Quantity (mg/tablet) |
|---|---|
| 6-MBPB (R-enantiomer) | 20.0 |
| MDMA | 10.0 |
| Starch | 45.0 |
| Microcrystalline cellulose | 35.0 |
| Polyvinylpyrrolidone (as 10% solution in water) | 4.0 |
| Sodium carboxymethyl starch | 4.5 |
| Magnesium stearate | 0.5 |
| Talc | 1.0 |

In one non-limiting embodiment, a tablet, comprising the components below, including an R-6-MBPB and dextroamphetamine, is prepared. The active ingredients, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

| Ingredient | Quantity (mg/tablet) |
|---|---|
| R-6-MBPB | 20.0 |
| Dextroamphetamine | 10.0 |
| Starch | 45.0 |
| Microcrystalline cellulose | 35.0 |
| Polyvinylpyrrolidone (as 10% solution in water) | 4.0 |
| Sodium carboxymethyl starch | 4.5 |
| Magnesium stearate | 0.5 |
| Talc | 1.0 |

In one non-limiting embodiment, a tablet, comprising the components below, including the R-enantiomer of 6-MBPB, is prepared. The active ingredients, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

| Ingredient | Quantity (mg/tablet) |
|---|---|
| 6-MBPB (70% S-enantiomer) | 20.0 |
| Starch | 45.0 |
| Microcrystalline cellulose | 35.0 |
| Polyvinylpyrrolidone (as 10% solution in water) | 4.0 |
| Sodium carboxymethyl starch | 4.5 |
| Magnesium stearate | 0.5 |
| Talc | 1.0 |

### 6-MBPB Extended-Release Formulations

Depending on the desired release profile, the pharmaceutical formulation, for example, an oral solid dosage form, may contain a suitable amount of controlled-release agents, extended-release agents, and/or modified-release agents (e.g., delayed-release agents). The pharmaceutical solid oral dosage forms comprising the active agents of the present invention described herein can be further formulated to provide a modified or controlled release of the active agents of the present invention. In some embodiments, the solid dosage forms described herein can be formulated as a delayed release dosage form such as an enteric-coated delayed release oral dosage forms, i.e., as an oral dosage form of a pharmaceutical composition as described herein which utilizes an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agents of the formulations described herein are delivered using a pulsatile dosage form. Pulsatile dosage forms comprising the active agents of the present invention described herein may be administered using a variety of formulations known in the art. For example, such formulations include those described in U.S. Pat. Nos. 5,011,692; 5,017,381; 5,229,135; and 5,840,329. Other dosage forms suitable for use with the active agents of the present invention are described in, for example, U.S. Pat. Nos. 4,871,549; 5,260,068; 5,260,069; 5,508,040; 5,567,441; and 5,837,284.

In one embodiment, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from at least about 2% to about 75% or less, preferably from at least about 2.5% to about 70% or less, or from at least about 40% to about 70% or less, by weight of the total dose of the active agents of the present invention in said formulation, in admixture with one or more binders.

In one embodiment, a coating for providing a controlled, delayed, or extended-release is applied to 6-MBPB or to a core containing 6-MBPB. The coating may comprise a pharmaceutically acceptable ingredient in an amount sufficient, e.g., to provide an extended release from e.g., about 1 hours to about 7 hours following ingestion before release of the active agent. Suitable coatings include one or more differentially degradable coatings such as, by way of example only, pH-sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the active agents of the present invention formulation.

Many other types of controlled/delayed/extended-release systems known to those of ordinary skill in the art and are suitable for use with the active agents of the present invention formulations described herein. Examples of such delivery systems include polymer-based systems, such as polylactic and polyglycolic acid, polyanhydrides and polycaprolactone, cellulose derivatives (e.g., ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725; 4,624,848; 4,968,509; 5,461,140; 5,456,923, 5,516,527; 5,622,721, 5,686,105; 5,700,410; 5,977,175; 6,465,014 and 6,932,983.

In certain embodiments, the controlled release systems may comprise the controlled/delayed/extended-release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric.

The controlled/delayed/extended-release systems may utilize a hydrophilic polymer, including a water-swellable polymer (e.g., a natural or synthetic gum). The hydrophilic polymer may be any pharmaceutically acceptable polymer which swells and expands in the presence of water to slowly release the active agents of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous-based system and is a spray-dried CAP psuedolatex with particles <1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include AS-LG (LF), which dissolves at pH 5, AS-MG (MF), which dissolves at pH 5.5, and AS-HG (HF), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Multilayer tablet delivery (e.g., such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (e.g., such as that used in the GeoMatrix™ technology) modifies the hydration/swelling rates of the core and reduces the surface area available for drug release. These partial coatings provide modulation of the drug dissolution profile: they reduce the release rate from the device and shift the typical time-dependent release rate toward constant release. This technology enables customized levels of controlled release of specific active agents and/or simultaneous release of two different active agents at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different active agents or dual release rate of the same drug in a single dosage form may be prepared by using hydrophilic and hydrophobic polymer matrices. Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In one embodiment, the dosage form is a solid oral dosage form which is an immediate release dosage form whereby >80% of the active agents of the present invention are released within 2 hours after administration. In other embodiments, the invention provides an (e.g., solid oral) dosage form that is a controlled release or pulsatile release dosage form. In such instances, the release may be, e.g., 30 to 60% of the active agents of the present invention particles by weight are released from the dosage form within about 2 hours after administration and about 90% by weight of the active agents of the present invention released from the dosage form, e.g., within about 4 hours after administration. In yet other embodiments, the dosage form includes at least one active agent in an immediate-release form and at least one active agent in the delayed-release form or sustained-release form. In yet other embodiments, the dosage form includes at least two active agents that are released at different rates as determined by in-vitro dissolution testing or via oral administration.

The various release dosage formulations discussed above, and others known to those skilled in the art can be characterized by their disintegration profile. A profile is characterized by the test conditions selected. Thus, the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cmax, Tmax, and AUC (0-t).

In certain embodiments, the controlled, delayed or extended-release of one or more of the active agents of the fixed-dose combinations of the invention may be in the form of a capsule having a shell comprising the material of the rate-limiting membrane, including any of the coating materials previously discussed, and filled with the active agents of the present invention particles. A particular advantage of this configuration is that the capsule may be prepared independently of the active agent of the present invention particles; thus, process conditions that would adversely affect the drug can be used to prepare the capsule.

Alternatively, the formulation may comprise a capsule having a shell made of a porous or a pH-sensitive polymer made by a thermal forming process. Another alternative is a capsule shell in the form of an asymmetric membrane, i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase separate by exchanging the solvent with a miscible non-solvent. In another embodiment, spray layered active agents of the present invention particles are filled in a capsule.

An exemplary process for manufacturing the spray layered the active agents of the present invention is the fluidized bed spraying process. The active agents of the present invention suspensions or the active agents of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agents of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agents of the present invention particles or the active agents of the present invention complex particles comprise about 30 to 70 wt % of the active agents of the present invention based on the total weight of the particles.

In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes at least 40 mg (or at least 100 mg or at least 200 mg) of the active agents of the present invention and has a total weight of less than 800 mg (or less than 700 mg). The capsule may contain a plurality of the active agents of the present invention-containing beads, for example, spray layered beads. In some embodiments, the beads are 12-25% the active agents of the present invention by weight. In some embodiments, some or all of the active agents of the present invention containing beads are coated with a coating comprising 6 to 15% (or 8 to 12%) of the total bead weight. Optimization work typically involves lower loading levels and the beads constitute 30 to 60% of the finished bead weight. The capsule may contain a granulated composition, wherein the granulated composition comprises the active agents of the present invention.

The capsule may provide pulsatile release of the active agents of the present invention oral dosage form. In one embodiment, the formulations comprise: (a) a first dosage unit comprising 6-MBPB that is released substantially immediately following oral administration of the dosage form to a patient; (b) a second dosage unit comprising 6-MBPB that is released approximately 2 to 6 hours following administration of the dosage form to a patient.

For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In other embodiments, the pulsatile release capsule contains a plurality of beads formulated for modified release and the at least one agent of the present invention is, for example, spray granulated for immediate release.

In some embodiments, the release of the active agents of the present invention particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, e.g., the spray layered particles. In one embodiment, the spray layered particles coated with the delayed and/or sustained release coatings can be filled in a modified release capsule in which both enteric-coated particles and immediate release particles of the present invention beads are filled into a soft gelatin capsule. Additional suitable excipients may also be filled with the coated particles in the capsule. The uncoated particles release the active agent of the present invention immediately upon administration while the coated particles do not release the active agent of the present invention until these particles reach the intestine. By controlling the ratios of the coated and uncoated particles, desirable pulsatile release profiles also may be obtained. In some embodiments, the ratios between the uncoated and the coated particles are e.g., 20/80, or 30/70, or 40/60, or 50/50, w/w to obtain desirable release.

In certain embodiments, spray layered active agents of the present invention can be compressed into tablets with commonly used pharmaceutical excipients. Any appropriate apparatus for forming the coating can be used to make the enteric coated tablets, e.g., fluidized bed coating using a Wurster column, powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms,” 1990.

In certain embodiments, the spray layered active agents of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agents of the present invention formulation into the gastrointestinal fluid. In other embodiments, the spray layered active agents of the present invention particles or spray layered active agents complex particles with enteric coatings described above and one or more excipients are dry blended and compressed into a mass, such as a tablet.

In certain embodiments, a pulsatile release of the active agent of the present invention formulation comprises a first dosage unit comprising a formulation made from the active agent of the present invention containing granules made from a spray drying or spray granulated procedure or a formulation made from the active agent of the present invention complex containing granules made from a spray drying or spray granulated procedure without enteric or sustained-release coatings and a second dosage unit comprising spray layered the active agent of the present invention particles or spray layered the active agent of the present invention complex particles with enteric or sustained-release coatings. In one embodiment, the active agent is wet or dry blended and compressed into a mass to make a pulsatile release tablet.

In certain embodiments, binding, lubricating and disintegrating agents are blended (wet or dry) to the spray layered active agent of the present invention to make a compressible blend. In one embodiment, the dosage unit containing 6-MBPB and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing 6-MBPB. In yet another embodiment, the dosage unit containing 6-MBPB is in the form of an overcoat and completely covers the second dosage unit containing the other pharmacological agent.

In one embodiment, the dosage unit containing 6-MBPB and the dosage unit containing another pharmacological agent are compressed separately and then compressed together to form a bilayer tablet. In yet another embodiment, the dosage unit containing the other pharmacological agent is in the form of an overcoat and completely covers the second dosage unit containing 6-MBPB. In yet another embodiment, the dosage unit containing 6-MBPB is in the form of an overcoat and completely covers the second dosage unit containing the other pharmacological agent.

### Systemic Formulations

The formulations of the present invention can include 6-MBPB for any of the disclosed indications in a form suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agents of the present invention can be dissolved at concentrations of greater than about 1 mg/ml using water-soluble beta cyclodextrins (e.g., beta-sulfobutyl-cyclodextrin and 2-hydroxypropyl-beta-cyclodextrin. Proper fluidity can be maintained, for example, by the use of a coating such as a lecithin, by the maintenance of the required particle size in the case of dispersions, and by the use of surfactants.

The formulations of the present invention suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The formulations of the present invention designed for extended-release via subcutaneous or intramuscular injection can avoid first-pass metabolism and lower dosages of the active agents of the present invention will be necessary to maintain plasma levels of about 50 ng/ml. In such formulations, the particle size of the active agents of the present invention and the range of the particle sizes of the active agents of the present invention particles can be used to control the release of the drug by controlling the rate of dissolution in fat or muscle.

In one embodiment, a pharmaceutical composition containing 6-MBPB or a pharmaceutically acceptable salt thereof is formulated into a dosage form suitable for parenteral use The dosage form may be selected from, but not limited to, a lyophilized powder, a solution, or a suspension (e.g., a depot suspension).

In one embodiment, a pharmaceutical composition containing 6-MBPB or a pharmaceutically acceptable salt thereof is formulated into a topical dosage form. The topical dosage form is selected from, but not limited to, a patch, a gel, a paste, a cream, an emulsion, a liniment, a balm, a lotion, and an ointment.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. Indirect techniques, which are generally preferred, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

### Non-Limiting Examples of Formulations for Systemic Delivery

In one non-limiting embodiment, a suppository, comprising 25 mg of S-6-MBPB, is prepared. The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

| Ingredient | Quantity (mg) |
|---|---|
| S-6-MBPB | 25.0 |
| Saturated fatty acid glycerides | 2000.0 |

In one non-limiting embodiment, a suppository, comprising 25 mg of R-6-MBPB, is prepared. The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

| Ingredient | Quantity (mg) |
|---|---|
| R-6-MBPB | 25.0 |
| Saturated fatty acid glycerides | 2000.0 |

In one non-limiting embodiment, a suppository, comprising 25 mg of 6-MBPB, is prepared. The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

| Ingredient | Quantity (mg) |
|---|---|
| 6-MBPB | 25.0 |
| Saturated fatty acid glycerides | 2000.0 |

In one non-limiting embodiment, a suspension comprising 50 mg of S-6-MBPB per 5.0 ml dose is prepared using the ingredients below. The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

| Ingredient | Amount | |
|---|---|---|
| S-5-MBPB | 50.0 | mg |
| Xanthan gum | 4.0 | mg |
| Sodium carboxymethyl cellulose (11%) | 50.0 | mg |
| Microcrystalline cellulose (89%) | 50 | mg |
| Sucrose | 1.75 | g |
| Sodium benzoate | 10.0 | mg |
| Flavor and Color | q.v. | |
| Purified water | To 5.0 | ml |

In one non-limiting embodiment, an intravenous formulation is prepared using the following ingredients:

| Ingredient | Amount | |
|---|---|---|
| R-6-MBPB | 250.0 | mg |
| Isotonic saline | 1000 | ml |

In one non-limiting embodiment, a topical formulation is prepared using the ingredients below. The white soft paraffin is heated until molten. The liquid paraffin and emulsifying wax are incorporated and stirred until dissolved. The active ingredient is added and stirring is continued until dispersed. The mixture is then cooled until solid.

| Ingredient | Amount (g) |
|---|---|
| 6-MBPB | 10.0 |
| Emulsifying Wax | 30.0 |
| Liquid Paraffin | 20.0 |
| White Soft Paraffin | To 100 |

In one non-limiting embodiment, a topical formulation is prepared using the ingredients below. The white soft paraffin is heated until molten. The liquid paraffin and emulsifying wax are incorporated and stirred until dissolved. The active ingredient is added and stirring is continued until dispersed. The mixture is then cooled until solid.

| Ingredient | Amount (g) |
|---|---|
| 6-MBPB (S-enantiomer) | 10.0 |
| Emulsifying Wax | 30.0 |
| Liquid Paraffin | 20.0 |
| White Soft Paraffin | To 100 |

In one embodiment, a sublingual or buccal tablet, comprising 10 mg of S-6-MBPB, is prepared using the following ingredients. The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

| Ingredient | Amount (mg/tablet) |
|---|---|
| S-6-MBPB | 10.0 |
| Glycerol | 210.5 |
| Water | 143.0 |
| Sodium Citrate | 4.5 |
| Polyvinyl Alcohol | 26.5 |
| Polyvinylpyrrolidone | 15.5 |

In one non-limiting embodiment, a liquid formulation for vaporization comprising R-6-MPBP, is prepared using the ingredients below. The active mixture is mixed and added to a liquid vaporization appliance.

| Ingredient | Quantity (units) | |
|---|---|---|
| 6-MPBP (R-enantiomer) | 500 | mg |
| Propylene Glycol | 2 | ml |
| Glycerin | 2 | ml |

In one non-limiting embodiment, a formulation of dry powder for insufflation is prepared comprising the components below. The active mixture is mixed with the lactose and the mixture is added to a dry powder inhaling appliance.

| Ingredient | Weight % |
|---|---|
| RS-6-MBPB | 5 |
| Lactose | 95 |

### 6-MBPB Prodrugs

In certain aspects, 6-MBPB is administered as a prodrug. Prodrugs are compounds that are metabolized or otherwise transformed inside the body to the active pharmacologic agent(s) of interest. Thus, prodrug will contain the “active” component (for example, 6-MBPB and a prodrug moiety). Examples include addition of amino acids to the amine, which can be removed within the body by esterases or similar enzymes, and reactions at the keto-group to form enol ethers, enol esters, and imines. Prodrugs are frequently (though not necessarily) pharmacologically less active or inactive until converted to the parent drug. This is done in the body by a chemical or biological reaction. In some cases, the moiety or chemicals formed from it may also have beneficial effects, including increasing therapeutic effects, decreasing undesirable side effects, or otherwise altering the pharmacokinetics or pharmacodynamics of the active drug. When the chemical formed from the prodrug moiety has beneficial effects that contribute to the overall beneficial effects of administering the prodrug, then the formed chemical is considered a “codrug.”

Types of prodrugs contemplated to be within the scope of the invention include compounds that are transformed in various organs or locations in the body (e.g., liver, kidney, G.I., lung, tissue) to release the active compound. For example, liver prodrugs will include active compounds conjugated with a polymer or chemical moiety that is not released until acted upon by liver cytochrome enzymes and CYP metabolism includes dealkylation, dehydrogenation, reduction, hydrolysis, oxidation, and the breakdown of aromatic rings. Kidney prodrugs will include active compounds conjugated to L-gamma-glutamyl or N-acetyl-L-gamma glutamic moieties so that they are metabolized by gamma-glutamyl transpeptidase before they are bioactive. Alternatively, the compounds may be conjugated to alkylglucoside moieties to create glycosylation-based prodrugs. Digestive or G.I. prodrugs will include those where an active compound is, e.g., formulated into microspheres or nanospheres that do not degrade until the spheres are subjected to an acidic pH; formulated with an amide that will resist biochemical degradation until colonic pH is achieved; or, conjugated with a linear polysaccharide such as pectin that will delay activation until the combination reaches the bacteria in the colon. Besides these exemplary prodrug forms, many others will be known to those of ordinary skill.

### 6-MBPB Combination Therapy

In certain embodiments an effective amount of 6-MBPB and one or more other active compounds can be provided to treat a host in need thereof.

In some aspects, 6-MBPB is formulated in a pharmaceutical preparation with other active compounds to increase therapeutic efficacy, decrease unwanted effects, increase stability/shelf-life, and/or alter pharmacokinetics. Such other active compounds include, but are not limited to antioxidants (such alpha-lipoate in acid or salt form, ascorbate in acid or salt form, selenium, or N-acetylcysteine); H2-receptor agonists or antagonists (such as famotidine); stimulants (such as dextroamphetamine, amphetamine, lisdexamphetamine, or methamphetamine); entactogens (such as MDMA); anti-inflammatories as ibuprofen or ketoprofen); matrix metalloproteinase inhibitors (such as doxycycline); NOS inhibitors (such as S-methyl-L-thiocitrulline); proton pump inhibitors (such as omeprazole); phosphodiesterase 5 inhibitors (such as sildenafil); drugs with cardiovascular effects (beta antagonists such as propranolol, mixed alpha and beta antagonists such as carvedilol, alpha antagonists such as prazosin, imidazoline receptor agonists such as rilmenidine or moxonidine; serotonin antagonists such as ketanserin or lisuride); norepinephrine transporter blockers (such as reboxetine); acetylcholine nicotinic receptor modulators (such as bupropion, hydroxybupropion, methyllycaconitine, memantine, or mecamylamine); gastrointestinal acidifying agents (such as ascorbic acid or glutamic acid hydrochloride); alkalinizing agents (such as sodium bicarbonate), NMDA receptor antagonists (such as ketamine); or serotonin receptor agonists (such as 5-methoxy-N-methyl-N-isopropyltryptamine, psilocin, or psilocybin). The ingredients may be in ion, freebase, or salt form and may be isomers or prodrugs.

The pharmacological agents that make up the combination therapy disclosed herein may be a combined dosage form or in separate dosage forms intended for substantially simultaneous administration. The pharmacological agents that make up the combination therapy may also be administered sequentially, with either therapeutic compound being administered by a regimen calling for two-step administration. The two-step administration regimen may call for sequential administration of the active agents or spaced-apart administration of the separate active agents.

The time period between the multiple administration steps may range from, a few minutes to several hours, depending upon the properties of each pharmacological agent, such as potency, solubility, bioavailability, plasma half-life and kinetic profile of the pharmacological agent. Circadian variation of the target molecule concentration may also determine the optimal dose interval. For example, a compound of the present invention may be administered while the other pharmacological agent is being administered (concurrent administration) or may be administered before or after other pharmacological agent is administered (sequential administration).

In cases where the two (or more) drugs are included in the fixed-dose combinations of the present invention are incompatible, cross-contamination can be avoided, e.g., by incorporation of the drugs in different drug layers in the oral dosage form with the inclusion of a barrier layer(s) between the different drug layers, wherein the barrier layer(s) comprise one or more inert/non-functional materials.

In certain preferred embodiments, the formulations of the present invention are fixed-dose combinations of a compound of the present invention or a pharmaceutically acceptable salt thereof and at least one other pharmacological agent. Fixed-dose combination formulations may contain, but are not limited to, the following combinations in the form of single-layer monolithic tablet or multi-layered monolithic tablet or in the form of a core tablet-in-tablet or multi-layered multi-disk tablet or beads inside a capsule or tablets inside a capsule.

In one embodiment, the fixed-dose combination is a therapeutically efficacious fixed-dose combinations of immediate-release formulations of compounds of 6-MBPB and other pharmacological agents.

In one embodiment, extended-release multi-layered matrix tablets are prepared using fixed-dose combinations of 6-MBPB with another pharmacological agent. For example, a hydrophilic polymer may comprise guar gum, hydroxypropylmethylcellulose, and xanthan gum as matrix formers. Lubricated formulations may be compressed by a wet granulation method.

Another embodiment of the invention includes multiple variations in the pharmaceutical dosages of each drug in the combination as further outlined below. Another embodiment of the invention includes various forms of preparations including using solids, liquids, immediate or delayed or extended-release forms. Many types of variations are possible as known to those skilled in the art.

Pharmaceutical Combinations with Dextroamphetamine

In one embodiment, 6-MBPB or a pharmaceutically acceptable salt thereof is formulated in a pharmaceutical composition that also contains dextroamphetamine or a pharmaceutically acceptable salt thereof in the amount of at least about 2 mg, 4 mg, 5 mg, 7 mg, 10 mg, 15 mg, 20 mg, or 25 mg. The required amount of dextroamphetamine will vary depending on the needs of the patient. The compound of 6-MBPB can be a racemic compound, an R- or S-enantiomer, or an enantiomerically enriched mixture of R- or S-enantiomers. In one embodiment, the compound of 6-MBPB is deuterated wherein one to five hydrogens have been replaced with deuterium.

In one embodiment, the ratio of dextroamphetamine (with or without salt) to 6-MBPB (with or without salt) is about 1:2, about 1:3, about 1:4, or about 1:5 by weight.

Pharmaceutical Combinations with MDMA

In one embodiment, 6-MBPB is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The required amount of MDMA will vary depending on the needs of the patient. The compound of 6-MBPB can be a racemic compound, an R- or S-enantiomer, or an enantiomerically enriched mixture of R- or S-enantiomers. In one embodiment, the compound of 6-MBPB is deuterated wherein one to five hydrogens have been replaced with deuterium.

In one embodiment, the ratio of MDMA (with or without salt) to 6-MBPB (with or without salt) is at least about 3:1, about 2:1, about 1:1, about 1:2, about 1:3, about 1:4, or about 1:5 by weight.

Pharmaceutical Combinations with Psilocybin

In one embodiment, 6-MBPB or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of 6-MBPB can be a racemic compound, an R- or S-enantiomer, or an enantiomerically enriched mixture of R- or S-enantiomers. In one embodiment, the compound of 6-MBPB is deuterated wherein one to five hydrogens have been replaced with deuterium.

### Non-Limiting Examples of Combination Formulations

In one non-limiting embodiment, a capsule comprising S-6-MBPB, R-6-MBPB, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| S-6-MBPB | 30.0 |
| R-6-MBPB | 10.0 |
| Amphetamine sulfate | 5.0 |
| Starch | 109.0 |
| Magnesium stearate | 1.0 |

In one non-limiting embodiment, a capsule comprising deuterated S-6-MBPB, R-6-MBPB, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| 6-MBPB (S-enantiomer, | 10.0 |
| D3-N-Deuterated) | |
| 6-MBPB (R-enantiomer, | 30.0 |
| D3-N-Deuterated) | |
| Amphetamine sulfate | 5.0 |
| Starch | 109.0 |
| Magnesium stearate | 1.0 |

In one non-limiting embodiment, a capsule, comprising R-6-MBPB, S-6-MBPB, and psilocybin hydrochloride, is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| R-6-MBPB | 10.0 |
| S-6-MBPB | 30.0 |
| Psilocybin hydrochloride | 2.0 |
| Alpha lipoic acid | 40.0 |
| Starch | 72.0 |
| Magnesium stearate | 1.0 |

It should be readily appreciated that the above formulation examples are illustrative only. Accordingly, it should be understood that reference to particular compounds(s) is likewise illustrative, and the compounds(s) in any of the non-limiting examples of combination formulations may be substituted by other compounds(s) of the invention. Likewise, any of the other active compounds (e.g., amphetamine sulfate or psilocybin hydrochloride as described above) may be substituted by a different other active compound, as may the inactive compounds.

Moreover, substitution of the compound by its prodrug, free base, salt, or hydrochloride salt shall be understood to provide merely an alternative embodiment still within the scope of the invention. Further, compositions within the scope of the invention should be understood to be open-ended and may include additional active or inactive compounds and ingredients.

The type of formulation employed for the administration of the compounds employed in the methods of the present invention generally may be dictated by the compound(s) employed, the type of pharmacokinetic profile desired from the route of administration and the compound(s), and the state of the patient.

### Dosage Regimes

6-MBPB or a pharmaceutically acceptable formulations of the present invention can be administered to the host in any amount, and with any frequency, that achieves the goals of the invention as used by the healthcare provider, or otherwise by the host in need thereof, typically a human, as necessary or desired.

In certain embodiments, the composition as described herein is provided only in a controlled counseling session, and administered only once, or perhaps 2, 3, 4, or 5 or more times in repeated counseling sessions to address a mental disorder as described herein.

In other embodiments, the composition as described herein is provided outside of a controlled counseling session, and perhaps self-administered, as needed to perhaps 2, 3, 4, or 5 or more times in to address a mental disorder as described herein.

In other embodiments, the composition of the present invention may be administered on a routine basis for mental wellbeing or for entactogenic treatment.

The compounds of the current invention can be administered in a variety of doses, routes of administration, and dosing regimens, based on the indication and needs of the patient. Non-limiting examples of therapeutic use include discrete psychotherapeutic sessions, ad libitum use for treatment of episodic disorders, and ongoing use for treatment of subchronic and chronic disorders.

### Psychotherapeutic Sessions

For some indications, the medicine is taken in discrete psychotherapy or other beneficial sessions. It is anticipated that these sessions will typically be separated by more than 5 half-lifes of the medicine and, for most patients, will typically occur only 1 to 5 times each year.

For these sessions, it will typically be desirable to induce clearly perceptible entactogenic effects that will facilitate fast therapeutic progress. Non-exhaustive examples of oral doses of medicine that produce clearly perceptible entactogenic effects include about 25 to about 100 mg of 6-MBPB.

It is anticipated that the medicine would be taken once or, more rarely, two or three times in a single therapeutic session. In these cases, it is common for each subsequent dose to be half of the previous dose or lower. Multiple doses within a session typically occur because either the patient's sensitivity to the medicine was unknown and too low of an initial dose was employed or because the patient is experiencing a productive session and it is desirable to extend the duration of therapeutic effects. Controlled release preparations may be used to lengthen the duration of therapeutic effects from a single administration of the medicine. In cases where multiple administrations are used in a session, it is anticipated that individual doses will be lower so that plasma concentrations remain within a desired therapeutic range.

Non-limiting, non-exhaustive examples of indications that may benefit from psychotherapeutic sessions include depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders. Also included as exemplary situations where an individual would benefit from a psychotherapeutic session are situations from a reduction of neuroticism or psychological defensiveness, an increase in openness to experience, an increase in creativity, or an increase in decision-making ability.

### Ad Libitum Use for Treatment of Episodic Disorders

For some indications, such as social anxiety, where the patient has need for relief from episodic occurrence of a disorder, it is anticipated that the medicine would be taken as needed but that uses should be separated by more than 5 half-lifes of the medicine to avoid bioaccumulation and formation of tolerance.

For treating episodic disorders, clearly perceptible entactogenic effects are often not desirable, as they can impair some aspects of functioning. Non-exhaustive examples of oral doses of medicine that produce subtle, barely perceptible therapeutic effects include about 5 to about 24 mg of 6-MBPB.

Non-limiting, non-exhaustive examples of indications that may benefit from episodic treatment are the same as those listed in the previous section provided that clinically significant signs and symptoms worsen episodically or in predictable contexts.

### Ongoing Use for Treatment of Subchronic and Chronic Disorders

For some indications, such as substance use disorders, inflammatory conditions, and neurological indications, including treatment of stroke, brain trauma, dementia, and neurodegenerative diseases, where the patient has need for ongoing treatment, it is anticipated that the medicine would be taken daily, twice daily, or three times per day. With some indications (subchronic disorders), such as treatment of stroke or traumatic brain injury, it is anticipated that treatment duration will be time-limited and dosing will be tapered when the patient has recovered. An example dose taper regimen is a reduction in dose of 10% of the original dose per week for nine weeks. With other, chronic disorders, such as dementia, it is anticipated that treatment will be continued as long as the patient continues to receive clinically significant benefits.

For treating subchronic and chronic disorders, clearly perceptible entactogenic effects are often not desirable. Non-exhaustive examples of oral doses of medicine that produce subtle, barely perceptible therapeutic effects with ongoing dosing include about 1 to about 9 mg of 6-MBPB.

Non-limiting, non-exhaustive examples of subchronic and chronic disorders that may benefit from regular treatment include migraine, headaches (e.g., cluster headache), neurodegenerative disorders, Alzheimer's disease, Parkinson's disease, schizophrenia, stroke, traumatic brain injury, phantom limb syndrome, and other conditions where increasing neuronal plasticity is desirable.

### IV. Methods to Treat Cns Disorders Including Mental Disorders and for Mental Enhancement with Adventageous Combinations

The present invention provides methods and uses for the treatment of CNS disorders, including, but not limited to, mental disorders as described herein, including post-traumatic stress and adjustment disorders, comprising administering a salt morphic form, morphic salt mixture, or specified salt mixture of a benzofuran compound described herein or a pharmaceutical composition prepared from a salt morphic form, morphic salt mixture, or specified salt mixture of a benzofuran compound described herein. These compounds display many pharmacological properties that are beneficial to their use as therapeutics and represent an improvement over existing therapeutics.

The present invention also provides, for example, methods for the treatment of disorders, including, but not limited to depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making.

In other embodiments, a salt morphic form, morphic salt mixture, or specified salt mixture described herein of a compound or composition of the present invention is provided in an effective amount to treat a host, typically a human, with a CNS disorder that can be either a neurological condition (one that is typically treated by a neurologist) or a psychiatric condition (one that is typically treated by a psychiatrist). Neurological disorders are typically those affecting the structure, biochemistry or cause electrical abnormalities of the brain, spinal cord or other nerves. Psychiatric conditions are more typically thought of as mental disorders, which are primarily abnormalities of thought, feeling or behavior that cause significant distress or impairment of personal functioning.

Thus, the disclosed compounds can be used in an effective amount to improve neurological or psychiatric functioning in a patient in need thereof. Neurological indications include, but are not limited to improved neuroplasticity, including treatment of stroke, brain trauma, dementia, and neurodegenerative diseases. MDMA has been reported to have an EC50 of 7.41 nM for promoting neuritogenesis and an Emax approximately twice that of ketamine, which has fast acting psychiatric benefits that are thought to be mediated by its ability to promote neuroplasticity, including the growth of dendritic spines, increased synthesis of synaptic proteins, and strengthening synaptic responses. FIG. S3. in Ly et al. (Cell reports 23, no. 11 (2018): 3170-3182, https://doi.org/10.1016/j.celrep.2018.05.022). The compounds of the current invention can similarly be considered psychoplastogens, that is, small molecules that are able to induce rapid neuroplasticity (Olson, 2018, Journal of experimental neuroscience, 12, 1179069518800508. https://doi.org/10.1177% 2F1179069518800508). For example, in certain embodiments, the disclosed compounds and compositions can be used to improve stuttering and other dyspraxias or to treat Parkinson's disease or schizophrenia.

In other embodiments, the compositions and salt morphic form, morphic salt mixture, or specified salt mixture described herein of compounds of the present invention may be used in an effective amount to treat a host, typically a human, to modulate an immune or inflammatory response. The salt morphic form, morphic salt mixture, or specified salt mixture of compounds disclosed herein alter extracellular serotonin, which is known to alter immune functioning. MDMA produces acute time-dependent increases and decreases in immune response.

The following nonlimiting examples are relevant to any of the disorders, indications, methods of use or dosing regimens described herein.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of S enantiomer is greater than about 99 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of S enantiomer is greater than about 95 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of S enantiomer is greater than about 90 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of S enantiomer is greater than about 85 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of S enantiomer is greater than about 80 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of S enantiomer is greater than about 75 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of S enantiomer is greater than about 70 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of S enantiomer is greater than about 65 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of S enantiomer is greater than about 60 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of S enantiomer is greater than about 55 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of S enantiomer is greater than about 55 or 60 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of R enantiomer is greater than about 95 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt mixed salt, isotopic derivative, or prodrug thereof, wherein the percent of R enantiomer is greater than about 90 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of R enantiomer is greater than about 85 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of R enantiomer is greater than about 80 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt or mixed salt, isotopic derivative, or prodrug thereof, wherein the percent of R enantiomer is greater than about 75 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of R enantiomer is greater than about 70 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of R enantiomer is greater than about 65 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of R enantiomer is greater than about 60 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of R enantiomer is greater than about 55 percent.

In certain embodiments, a host, for example a human, is treated with an effective amount of a salt morphic form, morphic salt mixture, or specified salt mixture described herein of an enantiomerically enriched mixture of enantiomers of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt, mixed salt, isotopic derivative, or prodrug thereof, wherein the percent of R enantiomer is greater than about 55 or 60 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of S enantiomer is greater than about 95 percent.

In certain embodiments, a host, for example a human, is treated with an effective amount of a salt morphic form, morphic salt mixture, or specified salt mixture described herein of an salt, isotopic derivative, or prodrug thereof, wherein the percent of S enantiomer is greater than about 90 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of S enantiomer is greater than about 85 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of S enantiomer is greater than about 80 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of S enantiomer is greater than about 75 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of S enantiomer is greater than about 70 percent.

In certain embodiments, a host, for example a human, is treated with an effective amount of a salt morphic form, morphic salt mixture, or specified salt mixture described herein of an salt, isotopic derivative, or prodrug thereof, wherein the percent of S enantiomer is greater than about 65 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of S enantiomer is greater than about 60 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of S enantiomer is greater than about 55 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of S enantiomer is greater than about 55 or 60 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of R enantiomer is greater than about 99 percent.

In certain embodiments, a host, for example a human, is treated with an effective amount of a salt morphic form, morphic salt mixture, or specified salt mixture described herein of an salt, isotopic derivative, or prodrug thereof, wherein the percent of R enantiomer is greater than about 95 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of R enantiomer is greater than about 90 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of R enantiomer is greater than about 85 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of R enantiomer is greater than about 80 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of R enantiomer is greater than about 75 percent.

In certain embodiments, a host, for example a human, is treated with an effective amount of a salt morphic form, morphic salt mixture, or specified salt mixture described herein of an salt, isotopic derivative, or prodrug thereof, wherein the percent of R enantiomer is greater than about 70 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of R enantiomer is greater than about 65 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of R enantiomer is greater than about 60 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, mixed salt, isotopic derivative, or prodrug thereof, wherein the percent of R enantiomer is greater than about 55 percent.

In certain embodiments, a host, for example a human, is treated with an effective amount of a salt morphic form, morphic salt mixture, or specified salt mixture described herein of an enantiomerically enriched mixture of enantiomers of compounds of Formula A, Formula B, Formula C, Formula D, Formula E, or Formula F, or a pharmaceutically acceptable salt, mixed salt, isotopic derivative, or prodrug thereof, wherein the percent of R enantiomer is greater than about 55 or 60 percent.

In certain embodiments, a host, for example a human, is treated with an effective amount of a salt morphic form, morphic salt mixture, or specified salt mixture described herein of an mixed salt, isotopic derivative, or prodrug thereof, wherein the percent of R enantiomer is greater than about 99 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 95 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 90 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 85 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 80 percent.

In certain embodiments, a host, for example a human, is treated with an effective amount of a salt morphic form, morphic salt mixture, or specified salt mixture described herein of an mixed salt, isotopic derivative, or prodrug thereof, wherein the percent of R enantiomer is greater than about 75 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 70 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 65 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 60 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 55 percent.

In certain embodiments, a host, for example a human, is treated with an effective amount of a salt morphic form, morphic salt mixture, or specified salt mixture described herein of an mixed salt, isotopic derivative, or prodrug thereof, wherein the percent of R enantiomer is greater than about 55 or 60 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 99 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 95 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 90 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 85 percent.

In certain embodiments, a host, for example a human, is treated with an effective amount of a salt morphic form, morphic salt mixture, or specified salt mixture described herein of an mixed salt, isotopic derivative, or prodrug thereof, wherein the percent of S enantiomer is greater than about 80 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 75 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 70 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 65 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 60 percent.

In certain embodiments, a host, for example a human, is treated with an effective amount of a salt morphic form, morphic salt mixture, or specified salt mixture described herein of an mixed salt, isotopic derivative, or prodrug thereof, wherein the percent of S enantiomer is greater than about 55 percent.

In certain embodiments, a host, for example a human, is treated with an effective amount of a salt morphic form, morphic salt mixture, or specified salt mixture described herein of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 55 or 60 percent.

The present invention also provides methods for modulating the CNS in a mammal in need thereof, including a human, by administering a pharmaceutically effective amount of a salt morphic form, morphic salt mixture, or specified salt mixture described herein of a compound of the present invention, including S-5-MAPB, R-5-MAPB, S-6-MAPB, and/or R-6-MAPB or a pharmaceutically acceptable salt or mixed salt thereof.

In some embodiments, a method is provided for modulating the CNS in a mammal in need thereof, including a human, by administering a pharmaceutically effective amount of 5-MBPB and/or 6-MBPB or a pharmaceutically acceptable salt thereof. In one embodiment, a method is provided for modulating the CNS in a mammal in need thereof, including a human, by administering a pharmaceutically effective amount of Formula A and/or Formula B or a pharmaceutically acceptable salt thereof. In one embodiment, a method is provided for modulating the CNS in a mammal in need thereof, including a human, by administering a pharmaceutically effective amount of Formula C and/or Formula D or a pharmaceutically acceptable salt thereof. In one embodiment, a method is provided for modulating the CNS in a mammal in need thereof, including a human, by administering a pharmaceutically effective amount of Formula E and/or

Formula F or a pharmaceutically acceptable salt thereof. In one embodiment, a method is provided for modulating the CNS in a mammal in need thereof, including a human, by administering a pharmaceutically effective amount of a compound of Formula I, Formula II, Formula III, Formula IV, Formula V, Formula VI, Formula VII, Formula VIII, Formula IX, Formula X, Formula XI, Formula XII, or Formula XIII or a pharmaceutically acceptable salt thereof. In one embodiment, a method is provided for modulating the CNS in a mammal in need thereof, including a human, by administering a pharmaceutically effective amount of a compound of Formula XI, Formula XII, and/or Formula XIII or a pharmaceutically acceptable salt thereof.

In one embodiment, a method is provided to treat diseases or disorders linked to inadequate functioning of neurotransmission in the CNS comprising administering 5-MBPB and 6-MBPB or a pharmaceutically acceptable salt thereof in a host in need thereof.

In one embodiment, a method is provided to treat diseases or disorders linked to inadequate functioning of neurotransmission in the CNS comprising administering 5-MBPB and 6-MBPB or a pharmaceutically acceptable salt thereof in a host in need thereof.

In one embodiment, a method is provided to treat diseases or disorders linked to inadequate functioning of neurotransmission in the CNS comprising administering Bk-5-MAPB and Bk-6-MAPB or a pharmaceutically acceptable salt thereof in a host in need thereof.

In one embodiment, a method is provided to treat diseases or disorders linked to inadequate functioning of neurotransmission in the CNS comprising administering Bk-5-MBPB and Bk-6-MBPB or a pharmaceutically acceptable salt thereof in a host in need thereof.

In one embodiment, a method is provided to treat diseases or disorders linked to inadequate functioning of neurotransmission in the CNS comprising administering Formula A and Formula B or a pharmaceutically acceptable salt thereof in a host in need thereof.

In one embodiment, a method is provided to treat diseases or disorders linked to inadequate functioning of neurotransmission in the CNS comprising administering Formula C and Formula D or a pharmaceutically acceptable salt thereof in a host in need thereof.

In one embodiment, a method is provided to treat diseases or disorders linked to inadequate functioning of neurotransmission in the CNS comprising administering Formula E and Formula F or a pharmaceutically acceptable salt thereof in a host in need thereof.

In one embodiment, a method is provided to treat diseases or disorders linked to inadequate functioning of neurotransmission in the CNS comprising administering a compound of Formula I, Formula II, Formula III, Formula IV, Formula V, Formula VI, Formula VII, Formula VIII, Formula IX, Formula X, Formula XI, Formula XII, or Formula XIII or a pharmaceutically acceptable salt thereof in a host in need thereof.

In one embodiment, a method is provided to treat diseases or disorders linked to inadequate functioning of neurotransmission in the CNS comprising administering a compound of Formula I, Formula II, Formula III, Formula IV, Formula V, Formula VI, Formula VII, Formula VIII, Formula IX, Formula X, Formula XI, Formula XII, or Formula XIII or a pharmaceutically acceptable salt thereof in a host in need thereof.

This invention also provides the use S-5-MAPB, R-5-MAPB, S-6-MAPB, and/or R-6-MAPB or a pharmaceutically acceptable salt or composition to treat a maladaptive response to perceived psychological threats. In one embodiment, S-5-MAPB, R-5-MAPB, S-6-MAPB, and/or R-6-MAPB or a pharmaceutically acceptable salt or composition is administered in the context of psychotherapy. In one embodiment, S-5-MAPB, R-5-MAPB, S-6-MAPB, and/or R-6-MAPB or a pharmaceutically acceptable salt or composition is administered as a stand-alone treatment.

This invention also provides the administration of an effective amount of 5-MBPB and/or 6-MBPB or a pharmaceutically acceptable salt or composition to a host, typically a human, to treat a maladaptive response to perceived psychological threats. In one embodiment, 5-MBPB and/or 6-MBPB or a pharmaceutically acceptable salt or composition is administered in the context of psychotherapy. In one embodiment, 5-MBPB and/or 6-MBPB or a pharmaceutically acceptable salt or composition is administered as a stand-alone treatment.

This invention also provides the use Formula A or Formula B or a pharmaceutically acceptable salt or composition in an effective amount to treat a maladaptive response to perceived psychological threats. In one embodiment, Formula A or Formula B or a pharmaceutically acceptable salt or composition is administered in the context of psychotherapy. In one embodiment, Formula A or Formula B or a pharmaceutically acceptable salt or composition is administered as a stand-alone treatment.

This invention also provides the use Formula C or Formula D or a pharmaceutically acceptable salt or composition to treat a maladaptive response to perceived psychological threats. In one embodiment, Formula C or Formula D or a pharmaceutically acceptable salt or composition is administered in the context of psychotherapy. In one embodiment, Formula C or Formula D or a pharmaceutically acceptable salt or composition is administered as a stand-alone treatment.

This invention also provides the use Formula E and/or Formula F or a pharmaceutically acceptable salt or composition to treat a maladaptive response to perceived psychological threats. In one embodiment, Formula E and/or Formula F or a pharmaceutically acceptable salt or composition is administered in the context of psychotherapy. In one embodiment, Formula E and/or Formula F or a pharmaceutically acceptable salt or composition is administered as a stand-alone treatment.

This invention also provides the use Bk-5-MAPB and/or Bk-6-MAPB or a pharmaceutically acceptable salt or composition to treat a maladaptive response to perceived psychological threats. In one embodiment, Bk-5-MAPB and/or Bk-6-MAPB or a pharmaceutically acceptable salt or composition is administered in the context of psychotherapy. In one embodiment, Bk-5-MAPB and/or Bk-6-MAPB or a pharmaceutically acceptable salt or composition is administered as a stand-alone treatment.

This invention also provides the use Bk-5-MBPB and/or Bk-6-MBPB or a pharmaceutically acceptable salt or composition to treat a maladaptive response to perceived psychological threats. In one embodiment, Bk-5-MBPB and/or Bk-6-MBPB or a pharmaceutically acceptable salt or composition is administered in the context of psychotherapy. In one embodiment, Bk-5-MBPB and/or Bk-6-MBPB or a pharmaceutically acceptable salt or composition is administered as a stand-alone treatment.

### Non-Limiting Examples of Pharmacotherapeutic Use

Psychotherapy, cognitive enhancement, or life coaching conducted with the salt morphic form, morphic salt mixture, or specified salt mixture described herein of compounds described herein employed as an adjunct (hereafter, “pharmacotherapy”) is typically conducted in widely spaced sessions with one, two, or rarely three or more administrations of an entactogen per session. These sessions can be as frequent as weekly, but are more often approximately monthly or even less frequently. In most cases, a small number of pharmacotherapy sessions, on the order of one to three, is needed for the patient to experience significant clinical progress, as indicated, for example, by a reduction in signs and symptoms of mental distress, by improvement in functioning in some domain of life, by arrival at a satisfactory solution to some problem, or by increased feelings of closeness to and understanding of some other person. In some embodiments, the psychotherapy, cognitive enhancement, or life coaching is conducted with an effective amount of enantiomerically enriched S-5-MAPB, R-5-MAPB, S-6-MAPB, and/or R-6-MAPB or a pharmaceutically acceptable salt thereof. In some embodiments, the psychotherapy, cognitive enhancement, or life coaching is conducted with an effective amount of enantiomerically enriched 5-MBPB and/or 6-MBPB or a pharmaceutically acceptable salt thereof. In some embodiments, the psychotherapy, cognitive enhancement, or life coaching is conducted with an effective amount of enantiomerically enriched Bk-5-MAPB and/or Bk-6-MAPB or a pharmaceutically acceptable salt thereof. Alternatively, the psychotherapy, cognitive enhancement, or life coaching is conducted with an effective amount of enantiomerically enriched Bk-5-MBPB and/or Bk-6-MBPB or a pharmaceutically acceptable salt thereof. In one embodiment, the psychotherapy, cognitive enhancement, or life coaching is conducted with an effective amount of enantiomerically enriched Formula A and/or Formula B or a pharmaceutically acceptable salt thereof. In one embodiment, the psychotherapy, cognitive enhancement, or life coaching is conducted with an effective amount of enantiomerically enriched Formula C and/or Formula D or a pharmaceutically acceptable salt thereof. In one embodiment, the psychotherapy, cognitive enhancement, or life coaching is conducted with an effective amount of enantiomerically enriched Formula E and/or Formula F or a pharmaceutically acceptable salt thereof.

In one embodiment, the psychotherapy, cognitive enhancement, or life coaching is conducted with an effective amount of Formula I, Formula II, Formula III, Formula IV, Formula V, Formula VI, Formula VII, Formula VIII, Formula IX, Formula X, Formula XI, Formula XII, and/or Formula XIII or a pharmaceutically acceptable salt thereof.

In one embodiment, the psychotherapy, cognitive enhancement, or life coaching is conducted with an effective amount of Formula XI, Formula XII, and/or Formula XIII or a pharmaceutically acceptable salt thereof.

The following sections provide detailed examples of pharmacotherapy. While common procedures are described, these are intended as illustrative, non-limiting examples. It is anticipated that the prescribing physician and therapy team may wish to specify different procedures than those described here based on their clinical judgment concerning the needs of the patient.

The example methods of treatment can also be modified with very minor changes to treat multiple patients at once, including couples or families. Hence, “patient” should be understood to mean one or more individuals.

Use of a Salt, Salt Mixtures, or Salt Morphic Form of a Compound or Composition of the Present Invention in Conjunction with Conventional Psychotherapy or Coaching

In one embodiment, the use of a salt morphic form, morphic salt mixture, or specified salt mixture described herein of a compound or composition of the present invention as pharmacotherapy is integrated into the patient's ongoing psychotherapy or coaching (hereafter abbreviated as “psychotherapy”). If a patient in need of the pharmacotherapy is not in ongoing psychotherapy, then psychotherapy may be initiated and the pharmacotherapy added later, after the prescribing physician and treating psychotherapist, physician, coach, member of the clergy, or other similar professional or someone acting under the supervision of such a professional (hereafter, “therapist”) agree that the pharmacotherapy is indicated and that there have been sufficient meetings between the patient and therapist to establish an effective therapeutic alliance.

If the patient is not experienced with the pharmacotherapy, a conversation typically occurs in which the therapist or other members of the therapy team addresses the patient's questions and concerns about the medicine and familiarizes the patient with the logistics of pharmacotherapy-assisted session. The therapist describes the kinds of experience that can be expected during the pharmacotherapy session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

The salt morphic form, morphic salt mixture, or specified salt mixture of compounds and compositions of the invention (or alternately herein for convenience, the “medicine”) is administered shortly before or during a scheduled psychotherapy session, with timing optionally selected so that therapeutic effects begin by the time the psychotherapy session begins. Either shortly before or after administration of the medicine, it is common for the therapist to provide some reminder of their mutual commitments and expected events during the session.

The psychotherapy session is carried out by the therapist, who, optionally, may be remote and in communication with the patient using a communication means suitable for telehealth or telemedicine, such as a phone, video, or other remote two-way communication method. Optionally, video or other monitoring of the patient's response or behavior is used to document or measure the session. The therapist uses their clinical judgment and available data to adjust the session to the needs of the patient. Many therapists view their responsibility as being to facilitate rather than direct the patient's experience. This may sometimes involve silent empathic listening, while other times it may include more active support to help the patient arrive at new perspectives on their life.

It is anticipated that the therapeutic effects of the medicine will allow the patient to make more rapid therapeutic progress than would normally be possible. These effects include decreased neuroticism and increased feelings of authenticity. Patients are often able to calmly contemplate actual or possible experiences that would normally be upsetting or even overwhelming. This can facilitate decision making and creativity in addition to mental wellness.

Optionally, the prescribing physician may allow a second or even third administration of the medicine or another psychotherapeutic agent in order to extend the therapeutic effects. Optionally, a pharmaceutical preparation with modified release is employed to make this unnecessary.

Because the duration of the scheduled psychotherapy session may be shorter than the therapeutic effects of the medicine, the therapist may suggest to the patient activities to support further psychotherapeutic progress after the psychotherapy session has ended. Alternatively, the therapist may continue to work with the patient until the therapeutic effects of the medicine have become clinically minimal.

In a subsequent non-pharmacological psychotherapy session, the therapist and patient will typically discuss the patient's experiences from the pharmacotherapy session and the therapist will often aid the patient in recalling the therapeutic effects and help them to incorporate the experiences into their everyday lives.

Pharmacotherapy sessions may be repeated as needed, based on the judgment of the treating physician and therapy team regarding the needs of the patient.

### Use of a Salt Morphic Form, Morphic Salt Mixture, or Specified Salt Mixture Described Herein of a Compound or Composition of the Present Invention Outside of Conventional Psychotherapy

In one embodiment, a salt morphic form, morphic salt mixture, or specified salt mixture of a compound or composition of the present invention is administered outside of a conventional psychotherapy. This example method is a broader, more flexible approach to pharmacotherapy that is not centered on supervision by a therapist. These pharmacotherapy sessions can take place in many different quiet and safe settings, including the patient's home. The setting is typically chosen to offer a quiet setting, with minimal disruptions, where the patient feels psychologically safe and emotionally relaxed. The setting may be the patient's home but may alternatively be a clinic, retreat center, or hotel room.

In one alternative embodiment, the medicine is taken by the patient regularly to maintain therapeutic concentrations of the active a salt morphic form, morphic salt mixture, or specified salt mixture of a compound in the blood. In another alternative embodiment, the medicine is taken, as needed, for defined psychotherapy sessions.

Optionally, a checklist may be followed to prepare the immediate environment to minimize distractions and maximize therapeutic or decision-making benefits. This checklist can include items such as silencing phones and other communications devices, cleaning and tidying the environment, preparing light refreshments, preparing playlists of appropriate music, and pre-arranging end-of-session transportation if the patient is not undergoing pharmacotherapy at home.

Before the pharmacotherapy session, there may be an initial determination of the therapeutic or other life-related goals (for example, decision-making, increasing creativity, or simply appreciation of life) that will be a focus of the session. These goals can optionally be determined in advance with support from a therapist.

Optionally, the therapist may help the patient select stimuli, such as photographs, videos, augmented or virtual reality scenes, or small objects such as personal possessions, that will help focus the patient's attention on the goals of the session or on the patient's broader life journey. As examples that are intended to be illustrative and not restrictive, these stimuli can include photographs of the patient from when they were young, which can increase self-compassion, or can include stimuli relating to traumatic events or phobias experienced by the patient, which can help the patient reevaluate and change their response to such stimuli. Optionally, the patient selects these stimuli without assistance (e.g., without the involvement of the therapist) or does not employ any stimuli. Optionally, stimuli are selected in real time by the therapist or an algorithm based on the events of the session with the goal of maximizing benefits to the patient.

If the patient is not experienced with the pharmacotherapy, a conversation occurs in which the therapist addresses the patient's questions and concerns about the medicine and familiarizes the patient with the logistics of a pharmacotherapy-assisted session. The therapist describes the kinds of experience that can be expected during the pharmacotherapy-assisted session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

Selected session goals and any commitments or other agreements regarding conduct between the patient and therapy team are reviewed immediately before administration of the medicine. Depending on the pharmaceutical preparation and route of administration, the therapeutic effects of the medicine usually begin within one hour. Typical therapeutic effects include decreased neuroticism and increased feelings of authenticity. Patients are often able to calmly contemplate experiences or possible experiences that would normally be upsetting or even overwhelming. This can facilitate decision making and creativity in addition to mental wellness.

Optionally, sleep shades and earphones with music or soothing noise may be used to reduce distractions from the environment. Optionally, a virtual reality or immersive reality system may be used to provide stimuli that support the therapeutic process. Optionally, these stimuli are preselected; optionally, they are selected in real time by a person or an algorithm based on events in the session with the goal of maximizing benefits to the patient. Optionally, a therapist or other person well-known to the patient is present or available nearby or via phone, video, or other communication method in case the patient wishes to talk, however the patient may optionally undergo a session without the assistance of a therapist. Optionally, the patient may write or create artwork relevant to the selected session goals. Optionally, the patient may practice stretches or other beneficial body movements, such as yoga (“movement activity”).

Optionally, in other embodiments the patient may practice movement activity that includes more vigorous body movements, such as dance or other aerobic activity. Movement activity also may make use of exercise equipment such as a treadmill or bicycle.

In some additional embodiments, the patient may be presented with music, video, auditory messages, or other perceptual stimuli. Optionally, these stimuli may be adjusted based on the movements or other measurable aspects of the patient. Such adjustment may be done by the therapist with or without the aid of a computer, or by a computer alone in response to said patient aspects, including by an algorithm or artificial intelligence, and “computer” broadly meaning any electronic tool suitable for such purposes, whether worn or attached to a patient (e.g., watches, fitness trackers, “wearables,” and other personal devices; biosensors or medical sensors; medical devices), whether directly coupled or wired to a patient or wirelessly connected (and including desktop, laptop, and notebook computers; tablets, smartphones, and other mobile devices; and the like), and whether within the therapy room or remote (e.g., cloud-based systems).

For example, measurable aspects of a patient (e.g., facial expression, eye movements, respiration rate, pulse rate, skin color change, patient voice quality or content, patient responses to questions) from these tools may be individually transformed into scores on standardized scales by subtracting a typical value and then multiplying by a constant and these scores may be further multiplied by constants and added together to create an overall score that can optionally be transformed by multiplication with a link function, such as the logit function, to create an overall score. This score may be used to select or adjust stimuli such as selecting music with higher or lower beats-per-minute or with faster or slower notes, selecting images, audio, or videos with different emotionality or autobiographical meaning, or selecting activities for the patient to engage in (such as specific movements, journaling prompts, or meditation mantras).

It should be readily appreciated that a patient can participate in numerous therapeutically beneficial activities, where such participation follows or is in conjunction with the administration of a salt morphic form, morphic salt mixture, or specified salt mixture of a compound or composition of the invention, including writing about a preselected topic, engaging in yoga or other movement activity, meditating, creating art, viewing of photographs or videos or emotionally evocative objects, using a virtual reality or augmented reality system, talking with a person, and thinking about a preselected problem or topic, and it should be understood that such participation can occur with or without the participation or guidance of a therapist.

Optionally, the prescribing physician may allow a second or even third administration of the medicine or another psychotherapeutic agent in order to extend the therapeutic effects. Optionally, a pharmaceutical preparation with modified release is employed to make this unnecessary.

The patient typically remains in the immediate environment until the acute therapeutic effects of the medicine are clinically minimal, usually within eight hours. After this point, the session is considered finished.

The treatment plan will often include a follow-up session with a therapist. This follow-up session occurs after the pharmacotherapy session has ended, often the next day but sometimes several days later. In this session, the patient discusses their experiences from the pharmacotherapy session with the therapist, who can aid them in recalling the therapeutic effects and help them to incorporate the experiences into their everyday lives.

Pharmacotherapy sessions may be repeated as needed, based on the judgment of the treating physician and therapy team regarding the needs of the patient.

### V. Pharmacological Properties of Morphic Forms, Salts, and Salt Mixtures of the Present Invention

Non-limiting examples of unwanted effects that can be minimized by carefully selecting the balance of enantiomers, morphic form, salt, or salt mixture include hallucinogenic effects, psychoactive effects (such as excess stimulation or sedation), physiological effects (such as transient hypertension or appetite suppression), toxic effects (such as to the brain or liver), effects contributing to abuse liability (such as euphoria or dopamine release), and/or other side effects.

The present invention includes compounds with beneficial selectivity profiles for neurotransmitter transporters. The balance of weakly activating NET (to reduce cardiovascular toxicity risk) and decreasing the DAT to SERT ratio over the racemate (to increase therapeutic effect relative to addictive liability) is a desirable feature of an entactogenic therapy displayed by the compounds and compositions of the present invention.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, 95% of the S-enantiomer. An enantiomerically enriched mixture of an R-enantiomer contains at least 55% of the R-enantiomer, and typically at least about 60%, 65%, 70%, 75%, 80%, 85%, 90%, 95% of the R-enantiomer. The specific ratio of S or R enantiomer can be selected for the need of the patient according to the health care specialist to balance the desired effect.

The term enantiomerically enriched mixture as used herein does not include either a racemic mixture or a pure or substantially pure enantiomer.

The present invention also provides new medical uses for the described compounds, including but not limited to, administration in an effective amount to a host in need thereof such as a human for depression, dysthymia, anxiety, generalized anxiety, social anxiety, panic, adjustment disorders, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism or dissociative disorders or any other disorder described herein, including in the Background. One particular treatment is for adjustment disorder, which is highly prevalent in society and currently insufficiently addressed. In nonlimiting aspects, the compound used in the treatment includes, for example, an enantiomerically enriched composition or substantially pure R- or S-enantiomer of 5-MAPB, 6-MAPB, 5-MBPB, 6-MBPB, 5-Bk-5-MAPB, 6-Bk-MAPB, Bk-5-MBPB, Bk-6-MBPB, or a combination thereof.

In certain embodiments a benzofuran derivatives of the current invention are direct 5-HT_{1B} agonists. Very few substances are known that are 5-HT_{1B} agonists and also 5-HT releasers and these have significant toxicities. For example, meta-chlorophenylpiperazine (mCPP) is one example but is anxiogenic and induces headaches, limiting any clinical use.

However, MDMA itself does not bind directly to the 5-HT_{1B} (Ray. 2010. PloS one, 5 (2), e9019). 5-HT_{1B} agonism is noteworthy because indirect stimulation of these receptors, secondary to elevated extracellular serotonin, has been hypothesized to be required for the prosocial effects of MDMA (Heifets et al. 2019. Science translational medicine, 11 (522)), while other aspects of entactogen effects have been attributed to monoamine release (e.g., Luethi & Liechti. 2020. Archives of toxicology, 94 (4), 1085-1133). Thus, the unique ratios of 5-HT_{1B} stimulation and monoamine release displayed by the disclosed compounds enable different profiles of therapeutic effects that appear not achieved by MDMA or other known entactogens.

The general pharmacology of entactogen enantiomers and enantiomeric compositions has been poorly understood to date. They have been difficult to separate, and it is not currently easily predicted what the therapeutic effects of individual enantiomers or enantiomerically enriched compositions might be based on individual complex receptor binding. Further, trends in the contribution of individual enantiomers often do not translate to other members of the same class of compounds. For example, the S-(+)-enantiomer of MDMA is more psychoactive than the R-(−)-enantiomer, but in 3,4-methylenedioxyamphetamine (MDA, differing from MDMA only by the absence of an N-methyl group), the S-(+)-enantiomer is less active than its corresponding R-(−)-enantiomer (Anderson et al., NIDA Res Monogr, 1978, 22:8-15; Nichols. J. Psychoactive Drugs, 1986, 18:305-13).

In the case of amphetamine, a non-entactogenic stimulant, it has been observed that an enantiomerically enriched mixture of enantiomers displays properties superior to the racemic mix or either enantiomer alone (Joyce et al., Psychopharmacology, 2007, 191:669-677). The drug Adderall is a paradigm example of a mixture of enantiomers of amphetamine. The mixture has equal parts racemic amphetamine and dextroamphetamine mixed salts (sulfate, aspartate, and saccharate) which results in an approximately 3:1 ratio between the dextroamphetamine and levoamphetamine. The two enantiomers are different enough to give Adderall an effect profile different from the racemate or the d-enantiomer. However, to date, it has not been reported or predictable what properties a mixture of enantiomers of the entactogenic compounds described herein would produce or how to use the mixture in therapy.

Understanding the pharmacology of the entactogen enantiomers was further complicated by the fact that the therapeutic effects of entactogens are not identical to the more readily identifiable psychoactive effects. Moreover, different enantiomers may differ in potency and activity in dissimilar and unpredictable ways. For instance, when the enantiomers of 3,4-methylenedioxy-N-ethylamphetamine (MDE) were compared in humans, it was concluded that the therapeutic effects of MDE were due to the S-(+)-enantiomer while the R-(−)-enantiomer primarily contributed to unwanted and toxic effects (Spitzer et al., Neuropharmacology, 2001, 41.2:263-271). In contrast, it has been argued that the R-(−)-enantiomer of MDMA may maintain the therapeutic effects of (+)-MDMA with a reduced side effect profile (Pitts et al., Psychopharmacology, 2018, 235.2:377-392). Thus, it is not possible to predict which enantiomers will best retain or provide therapeutic activity. While the enantiomers of 5-MAPB have been at least partially separated (Kadkhodaei et al. Journal of Separation Science, 2018, 41 (6): 1274-1286), to the inventor's knowledge, there have not yet been any studies characterizing the pharmacological effects of the isolated enantiomers of a benzofuran entactogen before this invention.

As described in the non-limiting illustrative Example 9, in one embodiment, the compounds of the present invention are rapid releasers of serotonin. This mechanism of action works in parallel with the inhibition of serotonin reuptake. The combination of inhibiting reuptake and increasing release significantly raises levels of serotonin and enhances therapeutic effect.

Further, select compounds of the present invention retain antagonism of the serotonin transporter (SERT), which is believed to be the principal mechanism of action for SSRIs. In this way the present invention provides compounds and methods that act in a similar way to the current standard of care for many CNS disorders including mental disorders, but do not present the crucial drawback of delayed onset.

Finally, the compounds of the present invention show a 5-HT selectivity pattern that is important to therapeutic use. Agonism of the 5-HT_{2A} receptor can cause feelings of fear and hallucinations, but agonism of 5-HT_{1B} is believed to be tied to the pro-social effects of entactogens.

Enantiomerically enriched compositions of the present invention can be selected to be poor agonists of 5-HT_{2A}, but exhibit activity toward 5-HT_{1B}. For example, as described in the non-limiting illustrative Example 6, the majority of the compounds do not exhibit 5-HT_{2A} agonist activity but do exhibit 5-HT_{1B} agonist activity in the nonlimiting range of approximately 5 to 0.05 μM, or even 3 to 0.10 μM. Importantly, 5-HT_{1B} agonist activity effect occurs through direct action on the receptor, rather than as an indirect consequence of serotonin release. This is an unexpected because this property has not been observed in an entactogen, including MDMA, before. In one embodiment, the selectivity of the 5-HT_{1B} receptor over 5-HT_{2A} receptor allows for a more relaxed and therapeutically productive experience for the patient undergoing treatment with a compound of the present invention.

In other embodiments, a compound or composition of the present invention is provided in an effective amount to treat a host, typically a human, with a CNS disorder that can be either a neurological condition (one that is typically treated by a neurologist) or a psychiatric condition (one that is typically treated by a psychiatrist). Neurological disorders are typically those affecting the structure, biochemistry, or normal electrical functions of the brain, spinal cord or other nerves. Psychiatric conditions are more typically thought of as mental disorders, which are primarily abnormalities of thought, feeling or behavior that cause significant distress or impairment of personal functioning.

Thus, the disclosed compounds can be used in an effective amount to improve neurological or psychiatric functioning in a patient in need thereof. Neurological indications include, but are not limited to improved neuroplasticity, including treatment of stroke, brain trauma, dementia, and neurodegenerative diseases. MDMA has an EC₅₀ of 7.41 nM for promoting neuritogenesis and an Emax approximately twice that of ketamine, which has fast acting psychiatric benefits that are thought to be mediated by its ability to promote neuroplasticity, including the growth of dendritic spines, increased synthesis of synaptic proteins, and strengthening synaptic responses (Ly et al. Cell reports 23, no. 11 (2018): 3170-3182; FIG. S3). The compounds of the current invention can similarly be considered psychoplastogens, that is, small molecules that are able to induce rapid neuroplasticity (Olson, 2018, Journal of experimental neuroscience, 12, 1179069518800508. https://doi.org/10.1177% 2F1179069518800508). For example, in certain embodiments, the disclosed compounds and compositions can be used to improve stuttering and other dyspraxias or to treat Parkinson's disease or schizophrenia.

In other embodiments, the compositions and compounds of the present invention may be used in an effective amount to treat a host, typically a human, to modulate an immune or inflammatory response. The compounds disclosed herein alter extracellular serotonin, which is known to alter immune functioning. MDMA produces acute time-dependent increases and decreases in immune response.

### VI. Pharmaceutical Compositions and Salts

The salts, salt mixtures, and morphic forms of compounds described herein can be administered in an effective amount as the neat chemical but are more typically administered as a pharmaceutical composition for a host, typically a human, in need of such treatment in an effective amount for any of the disorders described herein. The compounds or compositions disclosed herein may be administered orally, topically, systemically, parenterally, by inhalation, insufflation, or spray, mucosally (e.g., buccal, sublingual), sublingually, transdermally, rectally, intravenous, intra-aortal, intracranial, subdermal, intraperitoneal, intramuscularly, inhaled, intranasal, subcutaneous, transnasal, or by other means, in dosage unit formulations containing conventional pharmaceutically acceptable carriers. Such compositions are prepared in a manner well known in the pharmaceutical art and comprise at least one active compound. (See, e.g., Remington, 2005, Remington: The science and practice of pharmacy, 21st ed., Lippincott Williams & Wilkins.)

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, a suppository, a buccal or sublingual formulation, a parenteral formulation, an ophthalmic solution, or in a medical device. Some dosage forms, such as tablets and capsules, are subdivided into suitably sized unit doses containing appropriate quantities of the active components, e.g., an effective amount to achieve the desired purpose.

A “pharmaceutically acceptable composition” thus refers to at least one compound (which may be a mixture of enantiomers or diastereomers, as fully described herein) of the invention and a pharmaceutically acceptable vehicle, excipient, diluent or other carrier in an effective amount to treat a host, typically a human, who may be a patient.

In certain nonlimiting embodiments the pharmaceutical composition is a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 20, 25, 40, 50, 100, 125, 150, 200, 250, 300, 400, 500, 600, 700, or 750 mg of active compound, or its salt or mixed salt.

The pharmaceutical compositions described herein can be formulated into any suitable dosage form, including tablets, capsules, gelcaps, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, pills, powders, delayed-release formulations, immediate-release formulations, modified release formulations, extended-release formulations, pulsatile release formulations, multi particulate formulations, and mixed immediate release and controlled release formulations. Generally speaking, the composition should be administered in an effective amount to administer an amount of the active agents of the present invention achieves a plasma level commensurate with the concentrations found to be effective in vivo for a period of time effective to elicit a desired therapeutic effect without abuse liability.

In making the compositions employed in the present invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, solutions, slurries, syrups, aerosols (as a solid or in a liquid medium), ointments containing for example up to 10% by weight of the active compound, soft and hard gelatin capsules, suppositories, dry powders for inhalation, liquid preparations for vaporization and inhalation, topical preparations, transdermal patches, sterile injectable solutions, and sterile packaged powders. Compositions may be formulated as immediate release, controlled release, sustained (extended) release or modified release formulations.

The compositions of the present invention can be administered by multiple routes, which may differ in different patients according to their preference, co-morbidities, side effect profile, and other factors (IV, PO, transdermal, etc.). In one embodiment, the pharmaceutical composition includes the presence of other substances with the active drugs, known to those skilled in the art, such as fillers, carriers, gels, skin patches, lozenges, or other modifications in the preparation to facilitate absorption through various routes (such as, but not limited to, gastrointestinal, transdermal, etc.) and/or to extend the effect of the drugs, and/or to attain higher or more stable serum levels or to enhance the therapeutic effect of the active drugs in the combination.

In preparing a formulation, it may be necessary to mill the salt morphic form, morphic salt mixture, or specified salt mixture of an active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, but are not limited to, lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from at least about 0.05 to about 350 mg or less, more preferably at least about 5.0 to about 180 mg or less, of the active ingredients. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent, or excipient.

The active compounds are effective over a wide dosage range. For example, as-needed dosages normally fall within the range of at least about 0.01 to about 4 mg/kg or less. In the treatment of adult humans, the range of at least about 0.2 to about 3 mg/kg or less, in single dose, is especially preferred.

It will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way.

In some instances, dosage levels below the lower limit of the aforesaid range may be more than adequate, while in other cases still larger doses may be employed without causing any harmful side effects, provided for instance that such larger doses may be first divided into several smaller doses for administration.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

In one embodiment, a powder comprising the active agents of the present invention described herein may be formulated to comprise one or more pharmaceutical excipients and flavors. Such a powder may be prepared, for example, by mixing the active agents of the present invention and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

### Oral Formulations

In certain embodiments, any selected a salt morphic form, morphic salt mixture, or specified salt mixture of a compound(s) of the present invention is formulated in an effective amount in an pharmaceutically acceptable oral dosage form. In one embodiment, the compound(s) is 5-MAPB and/or 6-MAPB or a pharmaceutically acceptable salt thereof. In one embodiment, the compound(s) is 5-MBPB and/or 6-MBPB or a pharmaceutically acceptable salt thereof. In one embodiment, the compound(s) is Bk-5-MAPB and/or Bk-6-MAPB or a pharmaceutically acceptable salt thereof. In one embodiment, the compound(s) is Bk-5-MBPB and/or Bk-6-MBPB or a pharmaceutically acceptable salt thereof. In one embodiment, the compound(s) is Formula A and/or Formula B or a pharmaceutically acceptable salt thereof. In one embodiment, the compound(s) is Formula C and/or Formula D or a pharmaceutically acceptable salt thereof. In one embodiment, the compound(s) is Formula E and/or Formula F or a pharmaceutically acceptable salt thereof. In one embodiment, the compound(s) is a compound of Formula I, Formula II, Formula III, Formula IV, Formula V, Formula VI, Formula VII, Formula VIII, Formula IX, Formula X, Formula XI, Formula XII, or Formula XIII or a pharmaceutically acceptable salt thereof. Oral dosage forms may include, but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. The oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agent of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

Alternatively, the pharmaceutical solid dosage forms described herein can comprise the active agent or agents of the present invention (i.e., the “active agent(s)”; but for convenience herein, both “active agent” and “active agents” shall mean “active agent(s)” unless context clearly indicates that what is intended or would be suitable is only one agent or only two or more agents) and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the active agent of the present invention formulation. In one embodiment, some or all of the active agent of the present invention particles are coated. In another embodiment, some or all of the active agent of the present invention particles are microencapsulated. In yet another embodiment, some or all of the active agent of the present invention is amorphous material coated and/or microencapsulated with inert excipients. In still another embodiment, the active agent of the present invention particles are not microencapsulated and are uncoated.

Suitable carriers for use in the solid dosage forms described herein include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft- or hard-shell capsules and in tablet formulation, binders ensure that the tablet remains intact after compression and help assure blend uniformity prior to a compression or fill step. Materials suitable for use as binders in the solid dosage forms described herein include carboxymethylcellulose, methylcellulose (e.g., Methocel®), hydroxypropylmethylcellulose (e.g., Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are typically used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm3, e.g. Avicel®, powdered cellulose), and talc.

Suitable wetting agents for use in the solid dosage forms described herein include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in the solid dosage forms described here include polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, e.g., gum tragacanth and gum acacia, guar gum, xanthans, including xanthan gum, sugars, cellulosic, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, e.g., butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining superdisintegrants such as Croscarmellose sodium and different grades of microcrystalline cellulose in different ratios. To aid disintegration, sodium starch glycolate will be added.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as ethanol, isopropyl alcohol, hexadecyl alcohol, glycerol, and propylene glycol. Ethers, such as poly(ethylene glycol), petroleum hydrocarbons such as mineral oil and petrolatum, and water may also be used in suspension formulations.

In some embodiments, formulations are provided comprising particles of 5-MAPB and/or 6-MAPB and at least one dispersing agent or suspending agent for oral administration to a subject in need thereof. In some embodiments, formulations are provided comprising particles of 5-MBPB and/or 6-MBPB and at least one dispersing agent or suspending agent for oral administration to a subject in need thereof. In some embodiments, formulations are provided comprising particles of Bk-5-MAPB and/or Bk-6-MAPB and at least one dispersing agent or suspending agent for oral administration to a subject in need thereof. In some embodiments, formulations are provided comprising particles of Bk-5-MBPB and/or Bk-6-MBPB and at least one dispersing agent or suspending agent for oral administration to a subject in need thereof. In some embodiments, formulations are provided comprising particles of compounds of Formula A and/or Formula B and at least one dispersing agent or suspending agent for oral administration to a subject in need thereof. In some embodiments, formulations are provided comprising particles of compounds of Formula C and/or Formula D and at least one dispersing agent or suspending agent for oral administration to a subject in need thereof. In some embodiments, formulations are provided comprising particles of compounds of Formula E and/or Formula F and at least one dispersing agent or suspending agent for oral administration to a subject in need thereof. In some embodiments, formulations are provided comprising particles of Formula I, Formula II, Formula III, Formula IV, Formula V, Formula VI, Formula VII, Formula VIII, Formula IX, or Formula X or a pharmaceutically acceptable salt thereof and at least one dispersing agent or suspending agent for oral administration to a subject in need thereof. In some embodiments, formulations are provided comprising particles of Formula XI, Formula XII, or Formula XIII, or a pharmaceutically acceptable salt thereof and at least one dispersing agent or suspending agent for oral administration to a subject in need thereof.

The formulation may be a powder and/or granules for suspension, and upon admixture with water, a substantially uniform suspension is obtained. As described herein, the aqueous dispersion can comprise amorphous and non-amorphous particles consisting of multiple effective particle sizes such that the drug is absorbed in a controlled manner over time. In certain embodiments, the aqueous dispersion or suspension is an immediate-release formulation. In another embodiment, an aqueous dispersion comprising amorphous particles is formulated such that a portion of the particles of the present invention are absorbed within, e.g., about 0.75 hours after administration and the remaining particles are absorbed 2 to 4 hours after absorption of the earlier particles.

In other embodiments, addition of a complexing agent to the aqueous dispersion results in a larger span of the particles to extend the drug absorption phase of the active agent such that 50-80% of the particles are absorbed in the first hour and about 90% are absorbed by about 4 hours. Dosage forms for oral administration can be aqueous suspensions selected from the group including pharmaceutically acceptable aqueous oral dispersions, emulsions, solutions, and syrups. See, e.g., Singh et al., Encyclopedia of Pharm. Tech., 2nd Ed., 754-757 (2002). In addition to the active agents of the present invention particles, the liquid dosage forms may comprise additives, such as (a) disintegrating agents; (b) dispersing agents; (c) wetting agents; (d) at least one preservative; (e) viscosity enhancing agents; (f) at least one sweetening agent; and (g) at least one flavoring agent.

Examples of disintegrating agents for use in the aqueous suspensions and dispersions include a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corp., Parsippany, N.J.)).

In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; noncrystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908® or Poloxamine 908®).

Wetting agents (including surfactants) suitable for the aqueous suspensions and dispersions described herein are known in the art and include acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one embodiment, the aqueous liquid dispersion can comprise methylparaben and propylparaben in a concentration ranging from at least about 0.01% to about 0.3% or less methylparaben by weight to the weight of the aqueous dispersion and at least about 0.005% to about 0.03% or less propylparaben by weight to the total aqueous dispersion weight. In yet another embodiment, the aqueous liquid dispersion can comprise methylparaben from at least about 0.05 to about 0.1 or less weight % and propylparaben from at least about 0.01 to about 0.02 or less weight % of the aqueous dispersion.

Suitable viscosity enhancing agents for the aqueous suspensions or dispersions described herein include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid formulations of the present invention can also comprise inert diluents commonly used in the art, such as water or other solvents, solubilizing agents, emulsifiers, and/or sweeteners.

In one embodiment, the formulation for oral delivery is an effervescent powder containing 5-MAPB and/or 6-MAPB or a pharmaceutically acceptable salt thereof. In one embodiment, the formulation for oral delivery is an effervescent powder containing 5-MBPB and/or 6-MBPB or a pharmaceutically acceptable salt thereof. In one embodiment, the formulation for oral delivery is an effervescent powder containing Bk-5-MAPB and/or Bk-6-MAPB or a pharmaceutically acceptable salt thereof. In one embodiment, the formulation for oral delivery is an effervescent powder containing Bk-5-MBPB and/or Bk-6-MBPB or a pharmaceutically acceptable salt thereof. In one embodiment, the formulation for oral delivery is an effervescent powder containing Formula A and/or Formula B or a pharmaceutically acceptable salt thereof. In one embodiment, the formulation for oral delivery is an effervescent powder containing Formula C and/or Formula D or a pharmaceutically acceptable salt thereof. Effervescent salts have been used to disperse medicines in water for oral administration. In one embodiment, the formulation for oral delivery is an effervescent powder containing Formula E and/or Formula F or a pharmaceutically acceptable salt thereof. In one embodiment, the formulation for oral delivery is an effervescent powder containing a salt morphic form, morphic salt mixture, or specified salt mixture of a compound of Formula I, Formula II, Formula III, Formula IV, Formula V, Formula VI, Formula VII, Formula VIII, Formula IX, Formula X, Formula XI, Formula XII, or Formula XIII. Effervescent salts have been used to disperse medicines in water for oral administration. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

Tablets of the invention described here can be prepared by methods well known in the art. Various methods for the preparation of the immediate release, modified release, controlled release, and extended-release dosage forms (e.g., as matrix tablets, tablets having one or more modified, controlled, or extended-release layers, etc.) and the vehicles therein are well known in the art. Generally recognized compendia of methods include: Remington: The Science and Practice of Pharmacy, Alfonso R. Gennaro, Editor, 20th Edition, Lippincott Williams & Wilkins, Philadelphia, PA; and Sheth et al. (1980), Compressed tablets, in Pharmaceutical dosage forms, Vol. 1, edited by Lieberman and Lachtman, Dekker, NY.

In certain embodiments, solid dosage forms, e.g., tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include, e.g., spray drying, pan coating, melt granulation, granulation, fluidized bed spray drying or coating (e.g., Wurster coating), tangential coating, top spraying, tableting, extruding and the like.

Compressed tablets are solid dosage forms prepared by compacting the bulk blend the active agents of the present invention formulations described above. In various embodiments, compressed tablets which are designed to dissolve in the mouth will comprise one or more flavoring agents. In other embodiments, the compressed tablets will comprise a film surrounding a final compressed tablet. In some embodiments, the film coating can provide a delayed release of the active agents of the present invention formulation. In other embodiments, the film coating aids in patient compliance (e.g., Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, e.g., by placing the bulk blend of the active agents of the present invention formulation, described above, inside of a capsule. In some embodiments, the formulations of the present invention (non-aqueous suspensions and solutions) are placed in a soft gelatin capsule. In other embodiments, the formulations of the present invention are placed in standard gelatin capsules or non-gelatin capsules such as capsules comprising HPMC. In other embodiments, the formulations of the present invention are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the active agents of the present invention is delivered in a capsule form.

In certain embodiments, ingredients (including or not including the active agent) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, e.g., via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include U.S. Pat. Nos. 4,343,789; 5,340,591; 5,456,923; 5,700,485; 5,723,269; and U.S. Pub. No. 2004/0013734. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet.

### Preparation of Pharmaceutical Compositions

In certain aspects a salt, salt mixture, or morphic form of the present invention is used to prepare a pharmaceutical composition for administration to a patient in need thereof wherein the pharmaceutical composition is not the same salt, salt mixture, or morphic form, however, the salt, salt mixture, or morphic form imparted improved manufacturing properties or purity on the pharmaceutical composition. For example, a shelf stable morphic for m described herein can be reconstituted in a liquid and then used in the treatment of a patient or alternatively the liquid can then be dried or spray dried to afford another pharmaceutical composition.

### Extended-Release Formulations

Depending on the desired release profile, the pharmaceutical formulation, for example, an oral solid dosage form, may contain a suitable amount of controlled-release agents, extended-release agents, and/or modified-release agents (e.g., delayed-release agents). The pharmaceutical solid oral dosage forms comprising the active agents of the present invention described herein can be further formulated to provide a modified or controlled release of the active agents of the present invention. In some embodiments, the solid dosage forms described herein can be formulated as a delayed release dosage form such as an enteric-coated delayed release oral dosage forms, i.e., as an oral dosage form of a pharmaceutical composition as described herein which utilizes an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agents of the formulations described herein are delivered using a pulsatile dosage form. Pulsatile dosage forms comprising the active agents of the present invention described herein may be administered using a variety of formulations known in the art. For example, such formulations include those described in U.S. Pat. Nos. 5,011,692; 5,017,381; 5,229,135; and 5,840,329. Other dosage forms suitable for use with the active agents of the present invention are described in, for example, U.S. Pat. Nos. 4,871,549; 5,260,068; 5,260,069; 5,508,040; 5,567,441; and 5,837,284.

In one embodiment, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from at least about 2% to about 75% or less, preferably from at least about 2.5% to about 70% or less, or from at least about 40% to about 70% or less, by weight of the total dose of the active agents of the present invention in said formulation, in admixture with one or more binders.

In one embodiment, a coating for providing a controlled, delayed, or extended-release is applied to 5-MAPB and/or 6-MAPB or to a core containing 5-MAPB and/or 6-MAPB. In one embodiment, a coating for providing a controlled, delayed, or extended-release is applied to 5-MBPB and/or 6-MBPB or to a core containing 5-MBPB and/or 6-MBPB. In one embodiment, a coating for providing a controlled, delayed, or extended-release is applied to Bk-5-MAPB and/or Bk-6-MAPB or to a core containing Bk-5-MAPB and/or Bk-6-MAPB. In one embodiment, a coating for providing a controlled, delayed, or extended-release is applied to Bk-5-MBPB and/or Bk-6-MBPB or to a core containing Bk-5-MBPB and/or Bk-6-MBPB. In one embodiment, a coating for providing a controlled, delayed, or extended-release is applied to Formula A and/or Formula B or to a core containing Formula A and/or Formula B. In one embodiment, a coating for providing a controlled, delayed, or extended-release is applied to Formula C and/or Formula D or to a core containing Formula C and/or Formula D. In one embodiment, a coating for providing a controlled, delayed, or extended-release is applied to Formula E and/or Formula For to a core containing Formula E and/or Formula F. In one embodiment, a coating for providing a controlled, delayed, or extended-release is applied to Formula I, Formula II, Formula III, Formula IV, Formula V, Formula VI, Formula VII, Formula VIII, Formula IX, Formula X, Formula XI, Formula XII, or Formula XIII or to a core containing Formula I, Formula II, Formula III, Formula IV, Formula V, Formula VI, Formula VII, Formula VIII, Formula IX, Formula X, Formula XI, Formula XII, or Formula XIII.

The coating may comprise a pharmaceutically acceptable ingredient in an amount sufficient, e.g., to provide an extended release from e.g., about 1 hours to about 7 hours following ingestion before release of the active agent. Suitable coatings include one or more differentially degradable coatings such as, by way of example only, pH-sensitive coatings (enteric coatings) such as acrylic resins (e.g., Eudragit® EPO, Eudragit® L30D-55, Eudragit® FS 30D Eudragit® L100-55, Eudragit® L100, Eudragit® $100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the active agents of the present invention formulation.

Many other types of controlled/delayed/extended-release systems known to those of ordinary skill in the art and are suitable for use with the active agents of the present invention formulations described herein. Examples of such delivery systems include polymer-based systems, such as polylactic and polyglycolic acid, polyanhydrides and polycaprolactone, cellulose derivatives (e.g., ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725; 4,624,848; 4,968,509; 5,461,140; 5,456,923, 5,516,527; 5,622,721, 5,686,105; 5,700,410; 5,977,175; 6,465,014 and 6,932,983.

In certain embodiments, the controlled release systems may comprise the controlled/delayed/extended-release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate cellulose, trimellitate, ethyl hydroxypropylmethylcellulose phthalate, hydroxypropylmethylcellulose acetate succinate, polyvinyl acetate phthalate, and Cotteric.

The controlled/delayed/extended-release systems may utilize a hydrophilic polymer, including a water-swellable polymer (e.g., a natural or synthetic gum). The hydrophilic polymer may be any pharmaceutically acceptable polymer which swells and expands in the presence of water to slowly release the active agents of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous-based system and is a spray-dried CAP psuedolatex with particles <1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include AS-LG (LF), which dissolves at pH 5, AS-MG (MF), which dissolves at pH 5.5, and AS-HG (HF), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Multilayer tablet delivery (e.g., such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (e.g., such as that used in the GeoMatrix™ technology) modifies the hydration/swelling rates of the core and reduces the surface area available for drug release. These partial coatings provide modulation of the drug dissolution profile: they reduce the release rate from the device and shift the typical time-dependent release rate toward constant release. This technology enables customized levels of controlled release of specific active agents and/or simultaneous release of two different active agents at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different active agents or dual release rate of the same drug in a single dosage form may be prepared by using hydrophilic and hydrophobic polymer matrices. Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In one embodiment, the dosage form is a solid oral dosage form which is an immediate release dosage form whereby >80% of the active agents of the present invention are released within 2 hours after administration. In other embodiments, the invention provides an (e.g., solid oral) dosage form that is a controlled release or pulsatile release dosage form. In such instances, the release may be, e.g., 30 to 60% of the active agents of the present invention particles by weight are released from the dosage form within about 2 hours after administration and about 90% by weight of the active agents of the present invention released from the dosage form, e.g., within about 4 hours after administration. In yet other embodiments, the dosage form includes at least one active agent in an immediate-release form and at least one active agent in the delayed-release form or sustained-release form. In yet other embodiments, the dosage form includes at least two active agents that are released at different rates as determined by in-vitro dissolution testing or via oral administration.

The various release dosage formulations discussed above, and others known to those skilled in the art can be characterized by their disintegration profile. A profile is characterized by the test conditions selected. Thus, the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cmax, Tmax, and AUC (0-1).

In certain embodiments, the controlled, delayed or extended-release of one or more of the active agents of the fixed-dose combinations of the invention may be in the form of a capsule having a shell comprising the material of the rate-limiting membrane, including any of the coating materials previously discussed, and filled with the active agents of the present invention particles. A particular advantage of this configuration is that the capsule may be prepared independently of the active agent of the present invention particles; thus, process conditions that would adversely affect the drug can be used to prepare the capsule.

Alternatively, the formulation may comprise a capsule having a shell made of a porous or a pH-sensitive polymer made by a thermal forming process. Another alternative is a capsule shell in the form of an asymmetric membrane, i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase separate by exchanging the solvent with a miscible non-solvent. In another embodiment, spray layered active agents of the present invention particles are filled in a capsule.

An exemplary process for manufacturing the spray layered the active agents of the present invention is the fluidized bed spraying process. The active agents of the present invention suspensions or the active agents of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agents of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agents of the present invention particles, or the active agents of the present invention complex particles comprise about 30 to 70 wt % of the active agents of the present invention based on the total weight of the particles.

In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes at least 40 mg (or at least 100 mg or at least 200 mg) of the active agents of the present invention and has a total weight of less than 800 mg (or less than 700 mg). The capsule may contain a plurality of the active agents of the present invention-containing beads, for example, spray layered beads. In some embodiments, the beads are 12-25% the active agents of the present invention by weight. In some embodiments, some or all of the active agents of the present invention containing beads are coated with a coating comprising 6 to 15% (or 8 to 12%) of the total bead weight. Optimization work typically involves lower loading levels, and the beads constitute 30 to 60% of the finished bead weight. The capsule may contain a granulated composition, wherein the granulated composition comprises the active agents of the present invention.

The capsule may provide pulsatile release of the active agents of the present invention oral dosage form. In one embodiment, the formulations comprise: (a) a first dosage unit comprising 5-MBPB and/or 6-MBPB that is released substantially immediately following oral administration of the dosage form to a patient; (b) a second dosage unit comprising 5-MBPB and/or 6-MBPB that is released approximately 2 to 6 hours following administration of the dosage form to a patient.

The capsule may provide pulsatile release of the active agents of the present invention oral dosage form. In one embodiment, the formulations comprise: (a) a first dosage unit comprising 5-MAPB and/or 6-MAPB that is released substantially immediately following oral administration of the dosage form to a patient; (b) a second dosage unit comprising 5-MAPB and/or 6-MAPB that is released approximately 2 to 6 hours following administration of the dosage form to a patient.

In one embodiment, the formulation comprises: (a) a first dosage unit comprising a salt, a salt mixture, or salt morphic form of a compounds of Formula A and/or Formula B that is released substantially immediately following oral administration of the dosage form to a patient; (b) a second dosage unit comprising compounds of Formula A and/or Formula B that is released approximately 2 to 6 hours following administration of the dosage form to a patient.

In one embodiment, the formulations comprises: (a) a first dosage unit comprising a salt morphic form, morphic salt mixture, or specified salt mixture of a compounds of Formula C and/or Formula D that is released substantially immediately following oral administration of the dosage form to a patient; (b) a second dosage unit comprising compounds of Formula C and/or Formula D that is released approximately 2 to 6 hours following administration of the dosage form to a patient.

In one embodiment, the formulation comprises: (a) a first dosage unit comprising a salt morphic form, morphic salt mixture, or specified salt mixture of a compounds of Formula E and/or Formula F that is released substantially immediately following oral administration of the dosage form to a patient; (b) a second dosage unit comprising compounds of Formula E and/or Formula F that is released approximately 2 to 6 hours following administration of the dosage form to a patient.

In one embodiment, the formulation comprises: (a) a first dosage unit comprising Bk-5-MAPB and/or Bk-6-MAPB that is released substantially immediately following oral administration of the dosage form to a patient; (b) a second dosage unit comprising Bk-5-MAPB and/or Bk-6-MAPB that is released approximately 2 to 6 hours following administration of the dosage form to a patient.

In one embodiment, the formulation comprises: (a) a first dosage unit comprising Bk-5-MBPB and/or Bk-6-MBPB that is released substantially immediately following oral administration of the dosage form to a patient; (b) a second dosage unit comprising Bk-5-MBPB and/or Bk-6-MBPB that is released approximately 2 to 6 hours following administration of the dosage form to a patient.

In one embodiment, the formulation comprises: (a) a first dosage unit comprising a compound of Formula I, Formula II, Formula III, Formula IV, Formula V, Formula VI, Formula VII, Formula VIII, Formula IX, Formula X, Formula XI, Formula XII, or Formula XIII or a pharmaceutically acceptable salt thereof that is released substantially immediately following oral administration of the dosage form to a patient; (b) a second dosage unit comprising a compound of Formula I, Formula II, Formula III, Formula IV, Formula V, Formula VI, Formula VII, Formula VIII, Formula IX, Formula X, Formula XI, Formula XII, or Formula XIII or a pharmaceutically acceptable salt thereof that is released approximately 2 to 6 hours following administration of the dosage form to a patient.

For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In other embodiments, the pulsatile release capsule contains a plurality of beads formulated for modified release and the at least one agent of the present invention is, for example, spray granulated for immediate release.

In some embodiments, the release of the active agents of the present invention particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, e.g., the spray layered particles. In one embodiment, the spray layered particles coated with the delayed and/or sustained release coatings can be filled in a modified release capsule in which both enteric-coated particles and immediate release particles of the present invention beads are filled into a soft gelatin capsule. Additional suitable excipients may also be filled with the coated particles in the capsule. The uncoated particles release the active agent of the present invention immediately upon administration while the coated particles do not release the active agent of the present invention until these particles reach the intestine. By controlling the ratios of the coated and uncoated particles, desirable pulsatile release profiles also may be obtained. In some embodiments, the ratios between the uncoated and the coated particles are e.g., 20/80, or 30/70, or 40/60, or 50/50, w/w to obtain desirable release.

In certain embodiments, spray layered active agents of the present invention can be compressed into tablets with commonly used pharmaceutical excipients. Any appropriate apparatus for forming the coating can be used to make the enteric coated tablets, e.g., fluidized bed coating using a Wurster column, powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms,” 1990.

In certain embodiments, the spray layered active agents of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agents of the present invention formulation into the gastrointestinal fluid. In other embodiments, the spray layered active agents of the present invention particles or spray layered active agents complex particles with enteric coatings described above and one or more excipients are dry blended and compressed into a mass, such as a tablet.

In certain embodiments, a pulsatile release of the active agent of the present invention formulation comprises a first dosage unit comprising a formulation made from the active agent of the present invention containing granules made from a spray drying or spray granulated procedure or a formulation made from the active agent of the present invention complex containing granules made from a spray drying or spray granulated procedure without enteric or sustained-release coatings and a second dosage unit comprising spray layered the active agent of the present invention particles or spray layered the active agent of the present invention complex particles with enteric or sustained-release coatings. In one embodiment, the active agent is wet or dry blended and compressed into a mass to make a pulsatile release tablet.

In certain embodiments, binding, lubricating and disintegrating agents are blended (wet or dry) to the spray layered active agent of the present invention to make a compressible blend. In one embodiment, the dosage unit containing 5-MBPB and/or 6-MBPB and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing 5-MBPB and/or 6-MBPB. In yet another embodiment, the dosage unit containing 5-MBPB and/or 6-MBPB is in the form of an overcoat and completely covers the second dosage unit containing the other pharmacological agent.

In certain embodiments, binding, lubricating and disintegrating agents are blended (wet or dry) to the spray layered active agent of the present invention to make a compressible blend. In one embodiment, the dosage unit containing 5-MAPB and/or 6-MAPB and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing 5-MAPB and/or 6-MAPB. In yet another embodiment, the dosage unit containing 5-MAPB and/or 6-MAPB is in the form of an overcoat and completely covers the second dosage unit containing the other pharmacological agent.

In one embodiment, the dosage unit containing Bk-5-MAPB and/or Bk-6-MAPB and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing Bk-5-MAPB and/or Bk-6-MAPB. In yet another embodiment, the dosage unit containing Bk-5-MAPB and/or Bk-6-MAPB is in the form of an overcoat and completely covers the second dosage unit containing the other pharmacological agent.

In one embodiment, the dosage unit containing Bk-5-MBPB and/or Bk-6-MBPB and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing Bk-5-MBPB and/or Bk-6-MBPB. In yet another embodiment, the dosage unit containing Bk-5-MBPB and/or Bk-6-MBPB is in the form of an overcoat and completely covers the second dosage unit containing the other pharmacological agent.

In one embodiment, the dosage unit containing Formula A and/or Formula B and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing Formula A and/or Formula B. In yet another embodiment, the dosage unit containing Formula A and/or Formula B is in the form of an overcoat and completely covers the second dosage unit containing the other pharmacological agent.

In one embodiment, the dosage unit containing Formula C and/or Formula D and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing Formula C and/or Formula D. In yet another embodiment, the dosage unit containing Formula C and/or Formula D is in the form of an overcoat and completely covers the second dosage unit containing the other pharmacological agent.

In one embodiment, the dosage unit containing Formula E and/or Formula F and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing Formula E and/or Formula F. In yet another embodiment, the dosage unit containing Formula E and/or Formula F is in the form of an overcoat and completely covers the second dosage unit containing the other pharmacological agent.

In one embodiment, the dosage unit containing a compound of Formula I, Formula II, Formula III, Formula IV, Formula V, Formula VI, Formula VII, Formula VIII, Formula IX, Formula X, Formula XI, Formula XII, or Formula XIII and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing a compound of Formula I, Formula II, Formula III, Formula IV, Formula V, Formula VI, Formula VII, Formula VIII, Formula IX, Formula X, Formula XI, Formula XII, or Formula XIII. In yet another embodiment, the dosage unit containing a compound of Formula I, Formula II, Formula III, Formula IV, Formula V, Formula VI, Formula VII, Formula VIII, Formula IX, Formula X, Formula XI, Formula XII, or Formula XIII is in the form of an overcoat and completely covers the second dosage unit containing the other pharmacological agent.

### Systemic Formulations

The formulations of the present invention can include any selected compound of the present invention for any of the disclosed indications in a form suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agents of the present invention can be dissolved at concentrations of greater than about 1 mg/ml using water-soluble beta cyclodextrins (e.g., beta-sulfobutyl-cyclodextrin and 2-hydroxypropyl-beta-cyclodextrin. Proper fluidity can be maintained, for example, by the use of a coating such as a lecithin, by the maintenance of the required particle size in the case of dispersions, and by the use of surfactants.

The formulations of the present invention suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The formulations of the present invention designed for extended-release via subcutaneous or intramuscular injection can avoid first-pass metabolism and lower dosages of the active agents of the present invention will be necessary to maintain plasma levels of about 50 ng/ml. In such formulations, the particle size of the active agents of the present invention and the range of the particle sizes of the active agents of the present invention particles can be used to control the release of the drug by controlling the rate of dissolution in fat or muscle.

In one embodiment, a pharmaceutical composition containing 5-MAPB and/or 6-MAPB or a pharmaceutically acceptable salt thereof is formulated into a dosage form suitable for parenteral use. In one embodiment, a pharmaceutical composition containing 5-MBPB and/or 6-MBPB or a pharmaceutically acceptable salt thereof is formulated into a dosage form suitable for parenteral use. In one embodiment, pharmaceutical compositions containing compounds of Formula A and/or Formula B or a pharmaceutically acceptable salt thereof is formulated into a dosage form suitable for parenteral use. In one embodiment, pharmaceutical compositions containing compounds of Formula C and/or Formula D or a pharmaceutically acceptable salt thereof is formulated into a dosage form suitable for parenteral use. In one embodiment, pharmaceutical compositions containing Bk-5-MAPB and/or Bk-6-MAPB or a pharmaceutically acceptable salt thereof is formulated into a dosage form suitable for parenteral use. In one embodiment, pharmaceutical compositions containing Bk-5-MBPB and/or Bk-6-MBPB or a pharmaceutically acceptable salt thereof is formulated into a dosage form suitable for parenteral use. In one embodiment, pharmaceutical compositions containing compounds of Formula E and/or Formula F or a pharmaceutically acceptable salt thereof is formulated into a dosage form suitable for parenteral use. In one embodiment, pharmaceutical compositions containing a compound of Formula I, Formula II, Formula III, Formula IV, Formula V, Formula VI, Formula VII, Formula VIII, Formula IX, Formula X, Formula XI, Formula XII, or Formula XIII or a pharmaceutically acceptable salt thereof is formulated into a dosage form suitable for parenteral use. The dosage form may be selected from, but not limited to, a lyophilized powder, a solution, or a suspension (e.g., a depot suspension).

In one embodiment, a pharmaceutical composition containing 5-MBPB and/or 6-MBPB or a pharmaceutically acceptable salt thereof is formulated into a topical dosage form. In one embodiment, a pharmaceutical composition containing Bk-5-MAPB and/or Bk-6-MAPB or a pharmaceutically acceptable salt thereof is formulated into a topical dosage form. In one embodiment, a pharmaceutical composition containing 5-MAPB and/or 6-MAPB or a pharmaceutically acceptable salt thereof is formulated into a topical dosage form. In one embodiment, a pharmaceutical composition containing Bk-5-MBPB and/or Bk-6-MBPB or a pharmaceutically acceptable salt thereof is formulated into a topical dosage form. In one embodiment, a pharmaceutical composition containing Formula A and/or Formula B or a pharmaceutically acceptable salt thereof is formulated into a topical dosage form. In one embodiment, a pharmaceutical composition containing Formula C and/or Formula D or a pharmaceutically acceptable salt thereof is formulated into a topical dosage form. In one embodiment, a pharmaceutical composition containing Formula E and/or Formula F or a pharmaceutically acceptable salt thereof is formulated into a topical dosage form. In one embodiment, a pharmaceutical composition containing a compound of Formula I, Formula II, Formula III, Formula IV, Formula V, Formula VI, Formula VII, Formula VIII, Formula IX, Formula X, Formula XI, Formula XII, or Formula XIII or a pharmaceutically acceptable salt thereof is formulated into a topical dosage form. The topical dosage form is selected from, but not limited to, a patch, a gel, a paste, a cream, an emulsion, a liniment, a balm, a lotion, and an ointment.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. Indirect techniques, which are generally preferred, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

### Pharmaceutically Acceptable Salts

The compounds described herein, including enantiomerically enriched mixtures, can be administered if desired as a pharmaceutically acceptable salt or a mixed salt. A mixed salt may be useful to increase solubility of the active substances, to alter pharmacokinetics, or for controlled release or other objective. In other embodiments the salt is used as an intermediate in the manufacturing of a benzofuran compound for administration to a patient in need thereof.

In certain embodiments a 6-MBPB salt or salt mixture is provided.

The compounds of the present invention are amines and thus basic, and therefore, react with inorganic and organic acids to form pharmaceutically acceptable acid addition salts. In some embodiments, the compounds of the present invention as free amines are oily and have decreased stability at room temperature. In this case it may be preferable to convert the free amines to their pharmaceutically acceptable acid addition salts for ease of handling and administration because in some embodiments, the pharmaceutically acceptable salt is solid at room temperature.

Acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like. In one embodiment, the compounds of the present invention are administered as oxalate salts. In one embodiment of the present invention, the compounds are administered as phosphate salts.

Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, sethionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like.

Alternatively, exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, finnarate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, sethiona, hybenzate, hydrabamine, hydrobromide, hydrobromide/bromide, hydrochloride, hydroiodide, hydroxide, hydroxybenzoate, hydroxynaphthoate, iodide, isethionate, sethionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like. (See Berge et al. (1977) “Pharmaceutical Salts,” J. Pharm. Sci. 66:1-19.) Preferred pharmaceutically acceptable salts are those employing a hydrochloride anion.

In certain embodiments the pharmaceutically acceptable salt is selected from HCl, sulfate, aspartate, saccharate, phosphate, oxalate, acetate, gluconate, maleate, malate, citrate, mesylate, nitrate, tartrate, amino acid anion, benzenesulfonate, benzoate, besylate, bicarbonate, bitartrate, camsylate, carbonate, decanoate, edetate, esylate, fumarate, gluceptate, cluconate, clutamate, glycolate, hexanoate, hydroxynapthtoate, HI, isethionate, lactate, lactobionate, mandelate, methylsulfate, mucate, napsylate, octanoate, oleate, pamoate, pantothenate, phosphate, polycalacturonate, propionate, salicylate, stearate, sulfate, teoclate, tosylate, or a mixture thereof.

In certain embodiments the salt morphic form, morphic salt mixture, or specified salt mixture described herein is selected from HCl, HBr, H₂SO₄, H₃PO₄, HNO₃, methanesulfonic, succinic, oxalic, maleic, fumaric, saccharate, aspartate, L-Arginine, and L-Lysine or a mixture thereof.

In certain embodiments a benzofuran compound described herein is provided as a salt mixture wherein the salt mixture comprises HCl and HBr; HCl and H₂SO₄; HCl and H₃PO₄; HCl and HNO₃; HCl and methanesulfonic; HCl and succinic; HCl and oxalic; HCl and maleic; HCl and fumaric; HCl and saccharate; HCl and aspartate; HCl and L-Arginine; or HCl and L-Lysine.

In certain embodiments a benzofuran compound described herein is provided as a salt mixture wherein the salt mixture comprises HBr and H₂SO₄; HBr and H₃PO₄; HBr and HNO₃; HBr and methanesulfonic; HBr and succinic; HBr and oxalic; HBr and maleic; HBr and fumaric; HBr and saccharate; HBr and aspartate; HBr and L-Arginine; or HBr and L-Lysine.

In certain embodiments a benzofuran compound described herein is provided as a salt mixture wherein the salt mixture comprises H₂SO₄ and H₃PO₄; H₂SO₄ and HNO₃; H₂SO₄ and methanesulfonic; H₂SO₄ and succinic; H₂SO₄ and oxalic; H₂SO₄ and maleic; H₂SO₄ and fumaric; H₂SO₄ and saccharate; H₂SO₄ and aspartate; H₂SO₄ and L-Arginine; or H₂SO₄ and L-Lysine.

In certain embodiments a benzofuran compound described herein is provided as a salt mixture wherein the salt mixture comprises H₃PO₄ and HNO₃; H₃PO₄ and methanesulfonic; H₃PO₄ and succinic; H₃PO₄ and oxalic; H₃PO₄ and maleic; H₃PO₄ and fumaric; H₃PO₄ and saccharate; H₃PO₄ and aspartate; H₃PO₄ and L-Arginine; or H₃PO₄ and L-Lysine.

In certain embodiments a benzofuran compound described herein is provided as a salt mixture wherein the salt mixture comprises HNO₃ and methanesulfonic; HNO₃ and succinic; HNO₃ and oxalic; HNO₃ and maleic; HNO₃ and fumaric; HNO₃ and saccharate; HNO₃ and aspartate; HNO₃ and L-Arginine; or HNO₃ and L-Lysine.

In certain embodiments a benzofuran compound described herein is provided as a salt mixture wherein the salt mixture comprises methanesulfonic and succinic; methanesulfonic and oxalic; methanesulfonic and maleic; methanesulfonic and fumaric; methanesulfonic and saccharate; methanesulfonic and aspartate; methanesulfonic and L-Arginine; or methanesulfonic and L-Lysine.

In certain embodiments a benzofuran compound described herein is provided as a salt mixture wherein the salt mixture comprises succinic and oxalic; succinic and maleic; succinic and fumaric; succinic and saccharate; succinic and aspartate; succinic and L-Arginine; or succinic and L-Lysine.

In certain embodiments a benzofuran compound described herein is provided as a salt mixture wherein the salt mixture comprises oxalic and maleic; oxalic and fumaric; oxalic and saccharate; oxalic and aspartate; oxalic and L-Arginine; or oxalic and L-Lysine.

In certain embodiments a benzofuran compound described herein is provided as a salt mixture wherein the salt mixture comprises maleic and fumaric; maleic and saccharate; maleic and aspartate; maleic and L-Arginine; or maleic and L-Lysine.

In certain embodiments a benzofuran compound described herein is provided as a salt mixture wherein the salt mixture comprises fumaric and saccharate; fumaric and aspartate; fumaric and L-Arginine; or fumaric and L-Lysine.

In certain embodiments a benzofuran compound described herein is provided as a salt mixture wherein the salt mixture comprises saccharate and aspartate; saccharate and L-Arginine; or saccharate and L-Lysine.

In certain embodiments a benzofuran compound described herein is provided as a salt mixture wherein the salt mixture comprises aspartate and L-Arginine; or aspartate and L-Lysine.

In certain embodiments a benzofuran compound described herein is provided as a salt mixture wherein the salt mixture comprises L-Arginine and L-Lysine.
1. In certain embodiment a benzofuran compound of the present invention is provided as a HCl salt.
2. In certain embodiment a benzofuran compound of the present invention is provided as a HBr salt or a HBr salt mixed with a salt of embodiment 1.
3. In certain embodiment a benzofuran compound of the present invention is provided as a H₂SO₄ salt or a H₂SO₄ salt mixed with a salt of any one of embodiments 1 or 2.
4. In certain embodiment a benzofuran compound of the present invention is provided as a H₃PO₄ salt or a H₃PO₄ salt mixed with a salt of any one of embodiments 1 to 3.
5. In certain embodiment a benzofuran compound of the present invention is provided as a HNO₃ salt or a HNO₃ salt mixed with a salt of any one of embodiments 1 to 4.
6. In certain embodiment a benzofuran compound of the present invention is provided as a methanesulfonic salt or a methanesulfonic salt mixed with a salt of any one of embodiments 1 to 5.
7. In certain embodiment a benzofuran compound of the present invention is provided as a succinic salt or a succinic salt mixed with a salt of any one of embodiments 1 to 6.
8. In certain embodiment a benzofuran compound of the present invention is provided as an oxalic salt or an oxalic salt mixed with a salt of any one of embodiments 1 to 7.
9. In certain embodiment a benzofuran compound of the present invention is provided as a maleic salt or a maleic salt mixed with a salt of any one of embodiments 1 to 8.
10. In certain embodiment a benzofuran compound of the present invention is provided as a fumaric salt or a fumaric salt mixed with a salt of any one of embodiments 1 to 9.
11. In certain embodiment a benzofuran compound of the present invention is provided as a saccharate salt or a saccharate salt mixed with a salt of any one of embodiments 1 to 10.
12. In certain embodiment a benzofuran compound of the present invention is provided as an aspartate salt or an aspartate salt mixed with a salt of any one of embodiments 1 to 11.
13. In certain embodiment a benzofuran compound of the present invention is provided as a L-Arginine salt or a L-Arginine salt mixed with a salt of any one of embodiments 1 to 12.
14. In certain embodiment a benzofuran compound of the present invention is provided as a L-Lysine salt or a L-Lysine salt mixed with a salt of any one of embodiments 1 to 13.
15. The benzofuran compound of any one of embodiments 1-14, wherein the benzofuran compound is racemic.
16. The benzofuran compound of any one of embodiments 1-14, wherein the benzofuran compound is enantiomerically enriched as an R-enantiomer.
17. The benzofuran compound of any one of embodiments 1-14, wherein the benzofuran compound is enantiomerically enriched as an S-enantiomer.
18. The benzofuran compound of any one of embodiments 1-14, wherein the benzofuran compound is a substantially pure R-enantiomer.
19. The benzofuran compound of any one of embodiments 1-14, wherein the benzofuran compound is a substantially pure S-enantiomer.
20. A mixture of compounds of any one of embodiments 1-19 further comprising an additional benzofuran compound described herein.
21. The mixture of embodiment 20 wherein the additional benzofuran compound is a racemate.
22. The mixture of embodiment 20 wherein the additional benzofuran compound is enantiomerically enriched as an R-enantiomer.
23. The mixture of embodiment 20 wherein the additional benzofuran compound is enantiomerically enriched as an S-enantiomer.
24. The mixture of embodiment 20 wherein the additional benzofuran compound is a substantially pure R-enantiomer.
25. The mixture of embodiment 20 wherein the additional benzofuran compound is a substantially pure S-enantiomer.
26. The benzofuran compound or mixture of compounds of any one of embodiments 1-26, wherein the compound or mixture of compounds is selected from 5-MAPB, 6-MAPB, 5-MBPB, 6-MBPB, Bk-5-MAPB, Bk-6-MAPB, Bk-5-MBPB and Bk-6-MBPB.

In certain embodiments a mixture of salts is provided wherein there is about 5, 10, 15, 20, 25, 30, 35, 40, 45, 50, 55, 60, 65, 70, 75, 80, 85, 90, or 95% by mole of one salt and the remainder of the mixture is another salt (for example HCl and Oxalate). In other embodiments there is independently about 5, 10, 15, 20, 25, 30, 35, 40, 45, 50, 55, 60, 65, 70, 75, 80, 85, 90, or 95% of three different salts. In other embodiments there is independently about 5, 10, 15, 20, 25, 30, 35, 40, 45, 50, 55, 60, 65, 70, 75, 80, 85, 90, or 95% of four different salts. In other embodiments there is independently about 5, 10, 15, 20, 25, 30, 35, 40, 45, 50, 55, 60, 65, 70, 75, 80, 85, 90, or 95% of five different salts. In other embodiments there is independently about 5, 10, 15, 20, 25, 30, 35, 40, 45, 50, 55, 60, 65, 70, 75, 80, 85, 90, or 95% of six different salts.

In certain embodiments a tryptophan salt of a compound or salt mixture described herein is provided. For example in certain embodiments a L-tryptophan salt of a compound or salt mixture described herein is provided.

In certain embodiments a composition is provided comprising a mixture of enantiomers as described herein as a salt. For example, a composition comprising about 25% S-6-MAPB HCl Pattern 1A and about 75% R-6-MAPB HCl Pattern 1A or about 25% R-6-MAPB HCl Pattern 1A and about 75% S-6-MAPB HCl Pattern 1A. In other embodiments a composition is provided that contains a mixture of enantiomers and a mixture of salts. For example, a composition comprising about 75% S-6-MAPB HCl and about 25% R-6-MAPB oxalate. In other embodiments a composition is provided that contains a mixture of different enantiomers described herein and a mixture of salts. For example, a composition comprising about 25% S-6-MAPB sulfate, about 25% S-6-MAPB saccharate, about 25% R/S-6-MAPB sulfate, and about 25% R/S-6-MAPB aspartate. Or in another non-limiting example, a composition comprising about 25% R-6-MAPB sulfate, about 25% R-6-MAPB saccharate, about 25% R/S-6-MAPB sulfate, and about 25% R/S-6-MAPB aspartate.

Working Examples 12-15, 17-19, 21-24, and 26 provide nonlimiting examples of salts of exemplary compounds of the present invention or for use in the methods of the present invention. While salts of 5-MAPB or 6-MAPB are illustrated, any of the compounds described herein can be substituted, including but not limited to 5-MBPB, 6-MBPB, Bk-5-MAPB, Bk-6-MAPB, Bk-5-MBPB or Bk-6-MBPB. The compounds can be used as salts or mixed salts in enantiomerically enriched form, or in substantially pure enantiomeric form Nonlimiting examples are the oxalate and phosphate salts (and wherein MAPB is used solely for exemplary purposes for ease of drafting, but can be substituted for any of the other compounds herein):

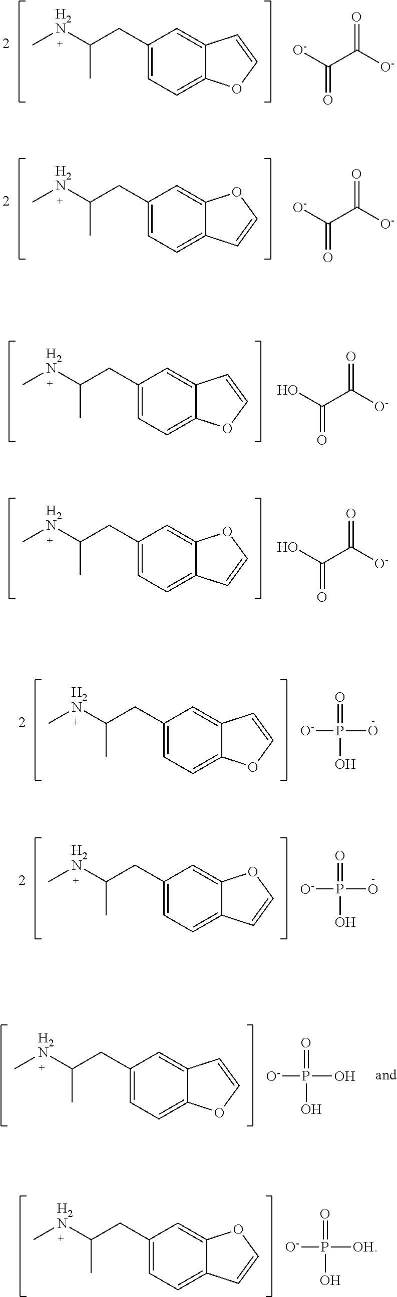

In certain illustrative nonlimiting embodiments, the pharmaceutically acceptable salt of 5-MAPB or 6-MAPB, including enantiomerically enriched 5-MAPB or 6-MAPB, is selected from:

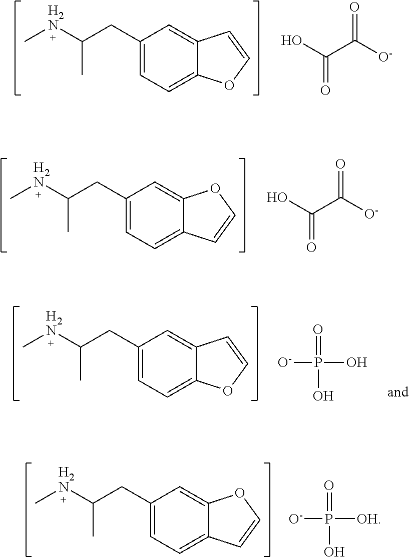

In certain illustrative nonlimiting embodiments, the pharmaceutically acceptable salt of 5-MAPB or 6-MAPB, including enantiomerically enriched 5-MAPB or 6-MAPB, is selected from:

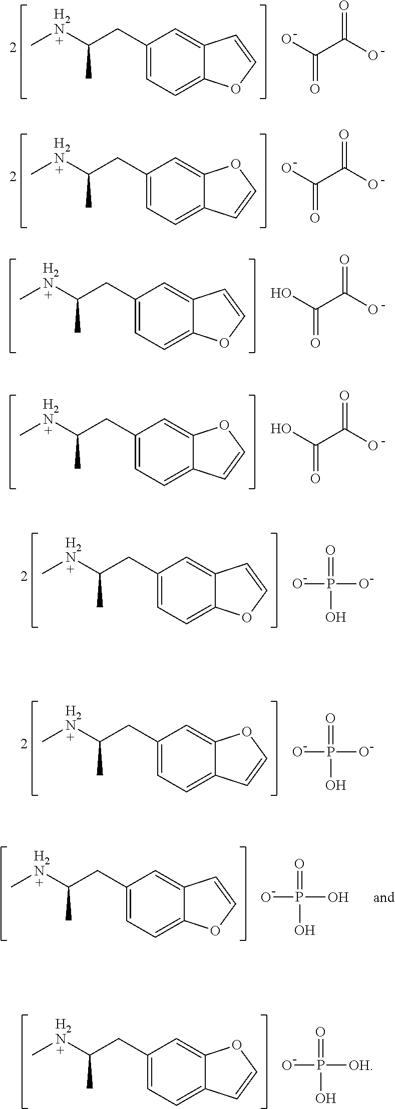

In certain illustrative nonlimiting embodiments, the pharmaceutically acceptable salt of 5-MAPB or 6-MAPB, including enantiomerically enriched 5-MAPB or 6-MAPB, is selected from:

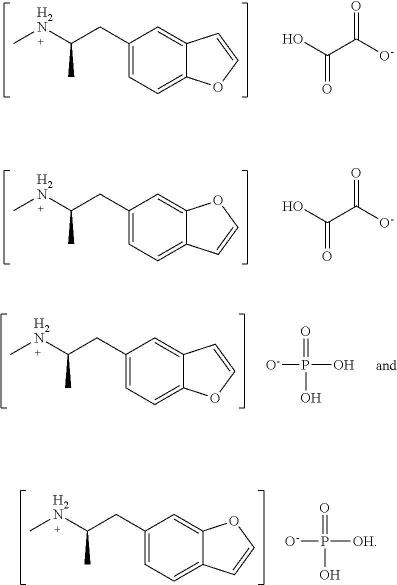

In certain illustrative nonlimiting embodiments, the pharmaceutically acceptable salt of 5-MAPB or 6-MAPB, including enantiomerically enriched 5-MAPB or 6-MAPB, is selected from:

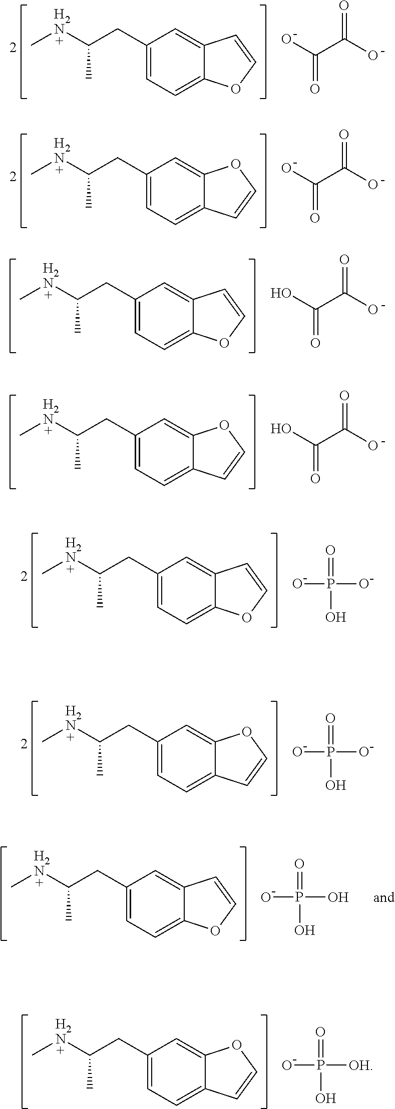

In certain illustrative nonlimiting embodiments, the pharmaceutically acceptable salt of 5-MAPB or 6-MAPB, including enantiomerically enriched 5-MAPB or 6-MAPB, is selected from:

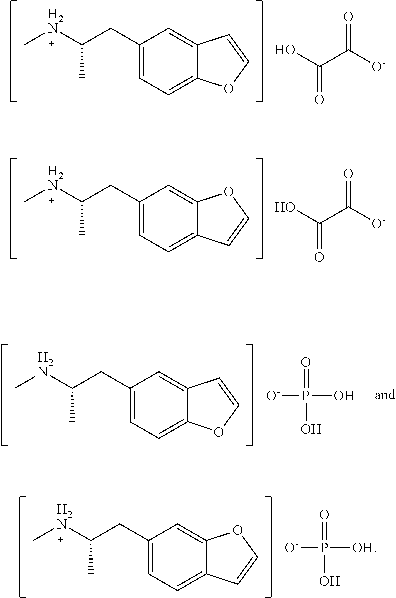

### VII. Combination Therapy

In certain embodiments, a pharmaceutical composition can be provided to the host, for example a human who can be a patient, with an effective amount of one or more other compounds either of the present invention or other active compounds, in combination, together with one or more other active compounds, and one or more pharmaceutically acceptable carriers, diluents, and/or excipients.

In some aspects, a compound of the present invention is formulated in a pharmaceutical preparation with other active compounds to increase therapeutic efficacy, decrease unwanted effects, increase stability/shelf-life, and/or alter pharmacokinetics. Such other active compounds include, but are not limited to antioxidants (such alpha-lipoate in acid or salt form, ascorbate in acid or salt form, selenium, or N-acetylcysteine); substrates or inhibitors of cytochrome p450 2D6 (such as dextromethorphan, fluoxetine, paroxetine, bupropion, duloxetine, or quinidine), H₂-receptor agonists or antagonists (such as famotidine); stimulants (such as dextroamphetamine, amphetamine, lisdexamphetamine, methylphenidate, or methamphetamine); entactogens (such as MDMA, 3,4-methylenedioxy-N-ethylamphetamine, [1-(2H-1,3-benzodioxol-5-yl) butan-2-yl](methyl)amine, 1-(1-benzofuran-6-yl) propan-2-amine, or [1-(1-benzofuran-5-yl) propan-2-yl](methyl)amine); anti-inflammatories (such as ibuprofen or ketoprofen); matrix metalloproteinase inhibitors (such as doxycycline); NOS inhibitors (such as S-methyl-L-thiocitrulline); proton pump inhibitors (such as omeprazole); phosphodiesterase 5 inhibitors (such as sildenafil); drugs with cardiovascular effects (beta antagonists such as propranolol, mixed alpha and beta antagonists such as carvedilol, alpha antagonists such as prazosin, imidazoline receptor agonists such as rilmenidine or moxonidine; serotonin antagonists such as ketanserin or lisuride);

norepinephrine transporter blockers (such as reboxetine); acetylcholine nicotinic receptor modulators (such as bupropion, hydroxybupropion, methyllycaconitine, memantine, or mecamylamine); gastrointestinal acidifying agents (such as ascorbic acid or glutamic acid hydrochloride); alkalinizing agents (such as sodium bicarbonate), NMDA receptor antagonists (such as ketamine); TrkB agonists (such as 7,8-dihydroxyflavone, 7,8,3′-trihydroxyflavone, or N-acetylserotonin), or serotonin receptor agonists (such as 5-methoxy-N-methyl-N-isopropyltryptamine, N,N-Dimethyl-2-(2-methyl-1H-indol-1-yl) ethan-1-amine, psilocin, or psilocybin). The ingredients may be in ion, freebase, or salt form and may be isomers or prodrugs.

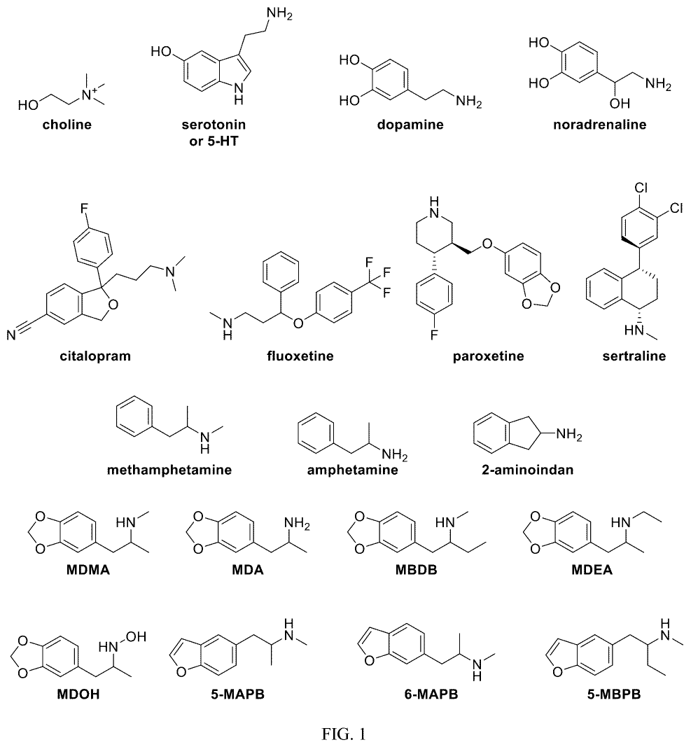

In certain embodiments the combination includes a compound selected from 5-MAPB, 6-MAPB, Bk-5-MAPB, Bk-6-MAPB, 5-MBPB, 6-MBPB, or a compound in FIG. 1, or a pharmaceutically acceptable salt or mixture there, optionally as a racemate, pure enantiomer, or an enantiomerically enriched mixture.

In certain embodiments the combination includes a compound selected from R-5-MAPB, S-5-MAPB, R-6-MAPB, S-6-MAPB, R-Bk-5-MAPB, S-Bk-5-MAPB, R-Bk-6-MAPB, S-Bk-6-MAPB, R-5-MBPB, S-5-MBPB, R-6-MBPB, or S-6-MBPB.

The pharmacological agents that make up the combination therapy disclosed herein may be a combined dosage form or in separate dosage forms intended for substantially simultaneous administration. The pharmacological agents that make up the combination therapy may also be administered sequentially, with either therapeutic compound being administered by a regimen calling for two-step administration. The two-step administration regimen may call for sequential administration of the active agents or spaced-apart administration of the separate active agents.

The time period between the multiple administration steps may range from, a few minutes to several hours, depending upon the properties of each pharmacological agent, such as potency, solubility, bioavailability, plasma half-life and kinetic profile of the pharmacological agent. Circadian variation of the target molecule concentration may also determine the optimal dose interval. For example, a compound of the present invention may be administered while the other pharmacological agent is being administered (concurrent administration) or may be administered before or after other pharmacological agent is administered (sequential administration).

In cases where the two (or more) drugs are included in the fixed-dose combinations of the present invention are incompatible, cross-contamination can be avoided, e.g., by incorporation of the drugs in different drug layers in the oral dosage form with the inclusion of a barrier layer(s) between the different drug layers, wherein the barrier layer(s) comprise one or more inert/non-functional materials.

In certain preferred embodiments, the formulations of the present invention are fixed-dose combinations of a compound of the present invention or a pharmaceutically acceptable salt thereof and at least one other pharmacological agent. Fixed-dose combination formulations may contain, but are not limited to, the following combinations in the form of single-layer monolithic tablet or multi-layered monolithic tablet or in the form of a core tablet-in-tablet or multi-layered multi-disk tablet or beads inside a capsule or tablets inside a capsule.

In one embodiment, the fixed-dose combination is a therapeutically efficacious fixed-dose combinations of immediate-release formulations of compounds of 5-MBPB and/or 6-MBPB and other pharmacological agents.

In one embodiment, the fixed-dose combination is a therapeutically efficacious fixed-dose combinations of extended-release compounds of 5-MBPB and/or 6-MBPB and delayed and/or extended-release other pharmacological agents contained in a single dosage form.

In one embodiment, the fixed-dose combination is a therapeutically efficacious fixed-dose combinations of immediate-release formulations of compounds of 5-MAPB and/or 6-MAPB and other pharmacological agents.

In one embodiment, the fixed-dose combination is a therapeutically efficacious fixed-dose combinations of extended-release compounds of 5-MAPB and/or 6-MAPB and delayed and/or extended-release other pharmacological agents contained in a single dosage form.

In one embodiment, the fixed-dose combination is a therapeutically efficacious fixed-dose combinations of immediate-release formulations of compounds of Formula A and/or Formula B and other pharmacological agents.

In one embodiment, the fixed-dose combination is a therapeutically efficacious fixed-dose combinations of extended-release compounds of Formula A and/or Formula B and delayed and/or extended-release other pharmacological agents contained in a single dosage form.

In one embodiment, the fixed-dose combination is a therapeutically efficacious fixed-dose combinations of immediate-release formulations of compounds of Formula C and/or Formula D and other pharmacological agents.

In one embodiment, the fixed-dose combination is a therapeutically efficacious fixed-dose combinations of extended-release compounds of Formula C and/or Formula D and delayed and/or extended-release other pharmacological agents contained in a single dosage form.

In one embodiment, the fixed-dose combination is a therapeutically efficacious fixed-dose combinations of immediate-release formulations of compounds of Bk-5-MAPB and/or Bk-6-MAPB and other pharmacological agents.

In one embodiment, the fixed-dose combination is a therapeutically efficacious fixed-dose combinations of extended-release compounds of Bk-5-MAPB and/or Bk-6-MAPB and delayed and/or extended-release other pharmacological agents contained in a single dosage form.

In one embodiment, the fixed-dose combination is a therapeutically efficacious fixed-dose combinations of immediate-release formulations of compounds of Bk-5-MBPB and/or Bk-6-MBPB and other pharmacological agents.

In one embodiment, the fixed-dose combination is a therapeutically efficacious fixed-dose combinations of extended-release compounds of Bk-5-MBPB and/or Bk-6-MBPB and delayed and/or extended-release other pharmacological agents contained in a single dosage form.

In one embodiment, the fixed-dose combination is a therapeutically efficacious fixed-dose combinations of immediate-release formulations of compounds of Formula E and/or Formula F and other pharmacological agents.

In one embodiment, the fixed-dose combination is a therapeutically efficacious fixed-dose combinations of extended-release compounds of Formula E and/or Formula F and delayed and/or extended-release other pharmacological agents contained in a single dosage form.

In one embodiment, the fixed-dose combination is a therapeutically efficacious fixed-dose combinations of immediate-release formulations of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof, and other pharmacological agents.

In one embodiment, the fixed-dose combination is a therapeutically efficacious fixed-dose combinations of immediate-release formulations of compounds of Formula XI, Formula XII, or Formula XIII, or a pharmaceutically acceptable salt thereof, and other pharmacological agents.

In one embodiment, the fixed-dose combination is a therapeutically efficacious fixed-dose combinations of extended-release formulations of compounds of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof, and other pharmacological agents.

In one embodiment, the fixed-dose combination is a therapeutically efficacious fixed-dose combinations of extended-release formulations of compounds of Formula XI, Formula XII, or Formula XIII, or a pharmaceutically acceptable salt thereof, and other pharmacological agents.

In one embodiment, the fixed-dose combination is a therapeutically efficacious fixed-dose combinations of immediate-release formulations of compounds of 5-MAPB and/or 6-MAPB and other pharmacological agents.

In one embodiment, extended-release multi-layered matrix tablets are prepared using fixed-dose combinations of 5-MAPB and/or 6-MAPB with another pharmacological agent. In one embodiment, extended-release multi-layered matrix tablets are prepared using fixed-dose combinations of 5-MBPB and/or 6-MBPB with another pharmacological agent. In one embodiment, extended-release multi-layered matrix tablets are prepared using fixed-dose combinations of Formula A and/or Formula B with another pharmacological agent. In one embodiment, extended-release multi-layered matrix tablets are prepared using fixed-dose combinations of Formula C and/or Formula D with another pharmacological agent. In one embodiment, extended-release multi-layered matrix tablets are prepared using fixed-dose combinations of Formula E and/or Formula F with another pharmacological agent. In one embodiment, extended-release multi-layered matrix tablets are prepared using fixed-dose combinations of Bk-5-MAPB and/or Bk-6-MAPB with another pharmacological agent. In one embodiment, extended-release multi-layered matrix tablets are prepared using fixed-dose combinations of Bk-5-MBPB and/or Bk-6-MBPB with another pharmacological agent. Such formulations may comprise one or more of the active agents within a hydrophilic or hydrophobic polymer matrix. In one embodiment, extended-release multi-layered matrix tablets are prepared using fixed-dose combinations of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof, with another pharmacological agent. In one embodiment, extended-release multi-layered matrix tablets are prepared using fixed-dose combinations of Formula XI, Formula XII, or Formula XIII, or a pharmaceutically acceptable salt thereof, with another pharmacological agent. In one embodiment, extended-release multi-layered matrix tablets are prepared using fixed-dose combinations of 5-MAPB and/or 6-MAPB with another pharmacological agent. For example, a hydrophilic polymer may comprise guar gum, hydroxypropylmethylcellulose, and xanthan gum as matrix formers. Lubricated formulations may be compressed by a wet granulation method.

Another embodiment of the invention includes multiple variations in the pharmaceutical dosages of each drug in the combination as further outlined below. Another embodiment of the invention includes various forms of preparations including using solids, liquids, immediate or delayed or extended-release forms. Many types of variations are possible as known to those skilled in the art.

In certain embodiments a morphic form, salt, or salt mixture of the present invention is administered in combination or alteration with L-tryptophan and/or 5-HTP.

Pharmaceutical Combinations with Dextroamphetamine

In one embodiment, 5-MBPB and/or 6-MBPB or a pharmaceutically acceptable salt thereof is formulated in a pharmaceutical composition that also contains dextroamphetamine or a pharmaceutically acceptable salt thereof in the amount of at least about 2 mg, 4 mg, 5 mg, 7 mg, 10 mg, 15 mg, 20 mg, or 25 mg. The required amount of dextroamphetamine will vary depending on the needs of the patient. The compound of 5-MBPB and/or 6-MBPB can be a racemic compound, an R- or S-enantiomer, or an enantiomerically enriched mixture of R- or S-enantiomers. In one embodiment, the compound of 5-MBPB and/or 6-MBPB is deuterated wherein one to five hydrogens have been replaced with deuterium.

In one embodiment, the ratio of dextroamphetamine (with or without salt) to 5-MBPB and/or 6-MBPB (with or without salt) is about 1:2, about 1:3, about 1:4, or about 1:5 by weight.

In one embodiment, 5-MAPB and/or 6-MAPB or a pharmaceutically acceptable salt thereof is formulated in a pharmaceutical composition that also contains dextroamphetamine or a pharmaceutically acceptable salt thereof in the amount of at least about 2 mg, 4 mg, 5 mg, 7 mg, 10 mg, 15 mg, 20 mg, or 25 mg. The required amount of dextroamphetamine will vary depending on the needs of the patient. The compound of 5-MAPB and/or 6-MAPB can be a racemic compound, an R- or S-enantiomer, or an enantiomerically enriched mixture of R- or S-enantiomers. In one embodiment, the compound of 5-MAPB and/or 6-MAPB is deuterated wherein one to five hydrogens have been replaced with deuterium.

In one embodiment, the ratio of dextroamphetamine (with or without salt) to 5-MAPB and/or 6-MAPB (with or without salt) is about 1:2, about 1:3, about 1:4, or about 1:5 by weight.

In one embodiment, a compound of Formula A and/or Formula B or a pharmaceutically acceptable salt thereof is formulated in a pharmaceutical composition that also contains dextroamphetamine or a pharmaceutically acceptable salt thereof in the amount of at least about 2 mg, 4 mg, 5 mg, 7 mg, 10 mg, 15 mg, 20 mg, or 25 mg. The required amount of dextroamphetamine will vary depending on the needs of the patient. The compound of Formula A and/or Formula B can be a racemic compound, an R- or S-enantiomer, or an enantiomerically enriched mixture of R- or S-enantiomers. In one embodiment, the compound of Formula A and/or B is deuterated wherein one to five hydrogens have been replaced with deuterium.

In one embodiment, the ratio of dextroamphetamine (with or without salt) to the compound of Formula A and/or Formula B (with or without salt) is about 1:2, about 1:3, about 1:4, or about 1:5 by weight.

In one embodiment, a compound of Formula C and/or Formula D or a pharmaceutically acceptable salt thereof is formulated in a pharmaceutical composition that also contains dextroamphetamine or a pharmaceutically acceptable salt of in the amount of at least about 2 mg, 4 mg, 5 mg, 7 mg, 10 mg, 15 mg, 20 mg, or 25 mg. The required amount of dextroamphetamine will vary depending on the needs of the patient. The compound of Formula C and/or Formula D can be a racemic compound, an R- or S-enantiomer, or an enantiomerically enriched mixture of R- or S-enantiomers. In one embodiment, the compound of Formula C and/or D is deuterated wherein one to five hydrogens have been replaced with deuterium.

In one embodiment, the ratio of dextroamphetamine (with or without salt) to the compound of Formula C and/or Formula D (with or without salt) is about 1:2, about 1:3, about 1:4, or about 1:5 by weight.

In one embodiment, Bk-5-MAPB and/or Bk-6-MAPB is formulated in a pharmaceutical composition that also contains dextroamphetamine or a pharmaceutically acceptable salt of in the amount of at least about 2 mg, 4 mg, 5 mg, 7 mg, 10 mg, 15 mg, 20 mg, or 25 mg. The required amount of dextroamphetamine will vary depending on the needs of the patient. The compound of Bk-5-MAPB and/or Bk-6-MAPB can be a racemic compound, an R- or S-enantiomer, or an enantiomerically enriched mixture of R- or S-enantiomers. In one embodiment, the compound of Bk-5-MAPB and/or Bk-6-MAPB is deuterated wherein one to five hydrogens have been replaced with deuterium.

In one embodiment, the ratio of dextroamphetamine (with or without salt) to Bk-5-MAPB and/or Bk-6-MAPB (with or without salt) is at least about 1:2, about 1:3, about 1:4, about 1:5, about 1:6, about 1:7, about 1:8, about 1:9, or about 1:10 by weight.

In one embodiment, Bk-5-MBPB and/or Bk-6-MBPB is formulated in a pharmaceutical composition that also contains dextroamphetamine or a pharmaceutically acceptable salt of in the amount of at least about 2 mg, 4 mg, 5 mg, 7 mg, 10 mg, 15 mg, 20 mg, or 25 mg. The required amount of dextroamphetamine will vary depending on the needs of the patient. The compound of Bk-5-MBPB and/or Bk-6-MBPB can be a racemic compound, an R- or S-enantiomer, or an enantiomerically enriched mixture of R- or S-enantiomers. In one embodiment, the compound of Bk-5-MBPB and/or Bk-6-MBPB is deuterated wherein one to five hydrogens have been replaced with deuterium.

In one embodiment, the ratio of dextroamphetamine (with or without salt) to Bk-5-MBPB and/or Bk-6-MBPB (with or without salt) is at least about 1:2, about 1:3, about 1:4, about 1:5, about 1:6, about 1:7, about 1:8, about 1:9, or about 1:10 by weight.

In one embodiment, a compound of Formula E and/or Formula F is formulated in a pharmaceutical composition that also contains dextroamphetamine or a pharmaceutically acceptable salt of in the amount of at least about 2 mg, 4 mg, 5 mg, 7 mg, 10 mg, 15 mg, 20 mg, or 25 mg. The required amount of dextroamphetamine will vary depending on the needs of the patient. The compound of Formula E and/or Formula F can be a racemic compound, an R- or S-enantiomer, or an enantiomerically enriched mixture of R- or S-enantiomers. In one embodiment, the compound of Formula E and/or F is deuterated wherein one to five hydrogens have been replaced with deuterium.

In one embodiment, the ratio of dextroamphetamine (with or without salt) to the compound of Formula E and/or Formula F (with or without salt) is at least about 1:2, about 1:3, about 1:4, about 1:5, about 1:6, about 1:7, about 1:8, about 1:9, or about 1:10 by weight.

In one embodiment, a compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof is formulated in a pharmaceutical composition that also contains dextroamphetamine or a pharmaceutically acceptable salt of in the amount of at least about 2 mg, 4 mg, 5 mg, 7 mg, 10 mg, 15 mg, 20 mg, or 25 mg. The required amount of dextroamphetamine will vary depending on the needs of the patient. The compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof, can be a racemic compound, an R- or S-enantiomer, or an enantiomerically enriched mixture of R- or S-enantiomers. In one embodiment, the compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof is deuterated wherein one to five hydrogens have been replaced with deuterium.

In one embodiment, the ratio of dextroamphetamine (with or without salt) to the compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof is at least about 1:2, about 1:3, about 1:4, about 1:5, about 1:6, about 1:7, about 1:8, about 1:9, or about 1:10 by weight.

In one embodiment, a compound of Formula XI, Formula XII, or Formula XIII, or a pharmaceutically acceptable salt thereof is formulated in a pharmaceutical composition that also contains dextroamphetamine or a pharmaceutically acceptable salt of in the amount of at least about 2 mg, 4 mg, 5 mg, 7 mg, 10 mg, 15 mg, 20 mg, or 25 mg. The required amount of dextroamphetamine will vary depending on the needs of the patient. The compound of Formula XI, Formula XII, or Formula XIII, or a pharmaceutically acceptable salt thereof, can be a racemic compound, an R- or S-enantiomer, or an enantiomerically enriched mixture of R- or S-enantiomers. In one embodiment, the compound of Formula X, Formula XI, Formula XII, or Formula XIII, or a pharmaceutically acceptable salt thereof is deuterated wherein one to five hydrogens have been replaced with deuterium.

In one embodiment, the ratio of dextroamphetamine (with or without salt) to the compound of Formula XI, Formula XII, or Formula XIII, or a pharmaceutically acceptable salt thereof is at least about 1:2, about 1:3, about 1:4, about 1:5, about 1:6, about 1:7, about 1:8, about 1:9, or about 1:10 by weight.

In one embodiment, 5-MAPB and/or 6-MAPB or a pharmaceutically acceptable salt thereof is formulated in a pharmaceutical composition that also contains dextroamphetamine or a pharmaceutically acceptable salt thereof in the amount of at least about 2 mg, 4 mg, 5 mg, 7 mg, 10 mg, 15 mg, 20 mg, or 25 mg. The required amount of dextroamphetamine will vary depending on the needs of the patient. The compound of 5-MAPB and/or 6-MAPB can be a racemic compound, an R- or S-enantiomer, or an enantiomerically enriched mixture of R- or S-enantiomers. In one embodiment, the compound of 5-MAPB and/or 6-MAPB is deuterated wherein one to five hydrogens have been replaced with deuterium.

In one embodiment, the ratio of dextroamphetamine (with or without salt) to 5-MAPB and/or 6-MAPB (with or without salt) is at least about 1:2, about 1:3, about 1:4, about 1:5, about 1:6, about 1:7, about 1:8, about 1:9, or about 1:10 by weight.

Pharmaceutical Combinations with MDMA

In one embodiment, 5-MBPB and/or 6-MBPB is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The required amount of MDMA will vary depending on the needs of the patient. The compound of 5-MBPB and/or 6-MBPB can be a racemic compound, an R- or S-enantiomer, or an enantiomerically enriched mixture of R- or S-enantiomers. In one embodiment, the compound of 5-MBPB and/or 6-MBPB is deuterated wherein one to five hydrogens have been replaced with deuterium.

In one embodiment, the ratio of MDMA (with or without salt) to 5-MBPB and/or 6-MBPB (with or without salt) is at least about 3:1, about 2:1, about 1:1, about 1:2, about 1:3, about 1:4, or about 1:5 by weight.

In one embodiment, 5-MAPB and/or 6-MAPB is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The required amount of MDMA will vary depending on the needs of the patient. The compound of 5-MAPB and/or 6-MAPB can be a racemic compound, an R- or S-enantiomer, or an enantiomerically enriched mixture of R- or S-enantiomers. In one embodiment, the compound of 5-MAPB and/or 6-MAPB is deuterated wherein one to five hydrogens have been replaced with deuterium.

In one embodiment, the ratio of MDMA (with or without salt) to 5-MAPB and/or 6-MAPB (with or without salt) is at least about 3:1, about 2:1, about 1:1, about 1:2, about 1:3, about 1:4, or about 1:5 by weight.

In one embodiment, Formula A and/or Formula B is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The compound of Formula A and/or Formula B can be a racemic compound, an R- or S-enantiomer, or an enantiomerically enriched mixture of R- or S-enantiomers. In one embodiment, the compound of Formula A and/or Formula B is deuterated wherein one to five hydrogens have been replaced with deuterium.

In one embodiment, the ratio of MDMA (with or without salt) to Formula A and/or Formula B (with or without salt) is at least about 3:1, about 2:1, about 1:1, about 1:2, about 1:3, about 1:4, or about 1:5 by weight.

In one embodiment, Formula C and/or Formula D is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The compound of Formula C and/or Formula D can be a racemic compound, an R- or S-enantiomer, or an enantiomerically enriched mixture of R- or S-enantiomers. In one embodiment, the compound of Formula C and/or Formula D is deuterated wherein one to five hydrogens have been replaced with deuterium.

In one embodiment, the ratio of MDMA (with or without salt) to Formula C and/or Formula D (with or without salt) is at least about 3:1, about 2:1, about 1:1, about 1:2, about 1:3, about 1:4, or about 1:5 by weight.

In one embodiment, Bk-5-MAPB and/or Bk-6-MAPB is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The compound of Bk-5-MAPB and/or Bk-6-MAPB can be a racemic compound, an R- or S-enantiomer, or an enantiomerically enriched mixture of R- or S-enantiomers. In one embodiment, the compound of Bk-5-MAPB and/or Bk-6-MAPB is deuterated wherein one to five hydrogens have been replaced with deuterium.

In one embodiment, the ratio of MDMA (with or without salt) to Bk-5-MAPB and/or Bk-6-MAPB (with or without salt) is at least about 3:1, about 2:1, about 1:1, about 1:2, about 1:3, about 1:4, or about 1:5 by weight.

In one embodiment, Bk-5-MBPB and/or Bk-6-MBPB is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The compound of Bk-5-MBPB and/or Bk-6-MBPB can be a racemic compound, an R- or S-enantiomer, or an enantiomerically enriched mixture of R- or S-enantiomers. In one embodiment, the compound of Bk-5-MBPB and/or Bk-6-MBPB is deuterated wherein one to five hydrogens have been replaced with deuterium.

In one embodiment, the ratio of MDMA (with or without salt) to Bk-5-MBPB and/or Bk-6-MBPB (with or without salt) is at least about 3:1, about 2:1, about 1:1, about 1:2, about 1:3, about 1:4, or about 1:5 by weight.

In one embodiment, Formula E and/or Formula F is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The compound of Formula E and/or Formula F can be a racemic compound, an R- or S-enantiomer, or an enantiomerically enriched mixture of R- or S-enantiomers. In one embodiment, the compound of Formula E and/or Formula F is deuterated wherein one to five hydrogens have been replaced with deuterium.

In one embodiment, the ratio of MDMA (with or without salt) to Formula E and/or Formula F (with or without salt) is at least about 3:1, about 2:1, about 1:1, about 1:2, about 1:3, about 1:4, or about 1:5 by weight.

In one embodiment, Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof can be a racemic compound, an R- or S-enantiomer, or an enantiomerically enriched mixture of R- or S-enantiomers. In one embodiment, the compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof is deuterated wherein one to five hydrogens have been replaced with deuterium.

In one embodiment, the ratio of MDMA (with or without salt) to Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof (with or without salt) is at least about 3:1, about 2:1, about 1:1, about 1:2, about 1:3, about 1:4, or about 1:5 by weight.

In one embodiment, Formula XI, Formula XII, or Formula XIII, or a pharmaceutically acceptable salt thereof is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The compound of Formula XI, Formula XII, or Formula XIII, or a pharmaceutically acceptable salt thereof can be a racemic compound, an R- or S-enantiomer, or an enantiomerically enriched mixture of R- or S-enantiomers. In one embodiment, the compound of Formula XI, Formula XII, or Formula XIII, or a pharmaceutically acceptable salt thereof is deuterated wherein one to five hydrogens have been replaced with deuterium.

In one embodiment, the ratio of MDMA (with or without salt) to Formula XI, Formula XII, or Formula XIII, or a pharmaceutically acceptable salt thereof (with or without salt) is at least about 3:1, about 2:1, about 1:1, about 1:2, about 1:3, about 1:4, or about 1:5 by weight.

Pharmaceutical Combinations with Psilocybin

In one embodiment, 5-MBPB and/or 6-MBPB or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of 5-MBPB and/or 6-MBPB can be a racemic compound, an R- or S-enantiomer, or an enantiomerically enriched mixture of R- or S-enantiomers. In one embodiment, the compound of 5-MBPB and/or 6-MBPB is deuterated wherein one to five hydrogens have been replaced with deuterium.

In one embodiment, a compound of Formula A and/or Formula B or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of Formula A and/or Formula B can be a racemic compound, an R- or S-enantiomer, or an enantiomerically enriched mixture of R- or S-enantiomers. In one embodiment, the compound of Formula A and/or B is deuterated wherein one to five hydrogens have been replaced with deuterium.

In one embodiment, a compound of Formula C and/or Formula D or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of Formula C and/or Formula D can be a racemic compound, an R- or S-enantiomer, or an enantiomerically enriched mixture of R- or S-enantiomers. In one embodiment, the compound of Formula C and/or D is deuterated wherein one to five hydrogens have been replaced with deuterium.

In one embodiment, Bk-5-MAPB and/or Bk-6-MAPB is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of Bk-5-MAPB and/or Bk-6-MAPB can be a racemic compound, an R- or S-enantiomer, or an enantiomerically enriched mixture of R- or S-enantiomers. In one embodiment, the compound of Bk-5-MAPB and/or Bk-6-MAPB is deuterated wherein one to five hydrogens have been replaced with deuterium.

In one embodiment, a compound of Formula E and/or Formula F is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of Formula E and/or Formula F can be a racemic compound, an R- or S-enantiomer, or an enantiomerically enriched mixture of R- or S-enantiomers. In one embodiment, the compound of Formula E and/or F is deuterated wherein one to five hydrogens have been replaced with deuterium.

In one embodiment, a compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof, can be a racemic compound, an R- or S-enantiomer, or an enantiomerically enriched mixture of R- or S-enantiomers. In one embodiment, the compound of Formula I, Formula II, Formula III, Formula IV, Formula V, Formula VI, Formula VII, Formula VIII, Formula IX, Formula X, Formula XI, Formula XII, or Formula XIII, or a pharmaceutically acceptable salt thereof is deuterated wherein one to five hydrogens have been replaced with deuterium.

In one embodiment, a compound of Formula XI, Formula XII, or Formula XIII, or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of Formula XI, Formula XII, or Formula XIII, or a pharmaceutically acceptable salt thereof, can be a racemic compound, an R- or S-enantiomer, or an enantiomerically enriched mixture of R- or S-enantiomers. In one embodiment, the compound of Formula X, Formula XI, Formula XII, or Formula XIII, or a pharmaceutically acceptable salt thereof is deuterated wherein one to five hydrogens have been replaced with deuterium.

In one embodiment, 5-MAPB and/or 6-MAPB or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of 5-MAPB and/or 6-MAPB can be a racemic compound, an R- or S-enantiomer, or an enantiomerically enriched mixture of R- or S-enantiomers. In one embodiment, the compound of 5-MAPB and/or 6-MAPB is deuterated wherein one to five hydrogens have been replaced with deuterium.

In one embodiment, Bk-5-MBPB and/or Bk-6-MBPB is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of Bk-5-MBPB and/or Bk-6-MBPB can be a racemic compound, an R- or S-enantiomer, or an enantiomerically enriched mixture of R- or S-enantiomers. In one embodiment, the compound of Bk-5-MBPB and/or Bk-6-MBPB is deuterated wherein one to five hydrogens have been replaced with deuterium.

### Non-Limiting Examples of Combination Formulations

In one non-limiting embodiment, a capsule comprising S-5-MAPB, R-5-MAPB, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| S-5-MAPB salt | 30.0 |
| R-5-MAPB salt | 10.0 |
| Amphetamine sulfate | 5.0 |
| Starch | 109.0 |
| Magnesium stearate | 1.0 |

In one non-limiting embodiment, a capsule comprising deuterated R-5-MBPB, R-6-MBPB, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| 5-MBPB (R-enantiomer, | 10.0 |
| D3-N-Deuterated) salt | |
| 6-MBPB (R-enantiomer, | 30.0 |
| D3-N-Deuterated) salt | |
| Amphetamine sulfate | 5.0 |
| Starch | 109.0 |
| Magnesium stearate | 1.0 |

In one non-limiting embodiment, a capsule, comprising a deuterated compound of Formula A, a deuterated compound of Formula B, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| Compound of Formula A (R-enantiomer, | 10.0 |
| D3-N-Deuterated) salt | |
| Compound of Formula B (R-enantiomer, | 30.0 |
| D3-N-Deuterated) salt | |
| Amphetamine sulfate | 5.0 |
| Starch | 109.0 |
| Magnesium stearate | 1.0 |

In one non-limiting embodiment, a capsule, comprising a deuterated compound of Formula C, a deuterated compound of Formula D, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| compound of Formula C (R-enantiomer, | 10.0 |
| D3-N-Deuterated) salt | |
| compound of Formula D (R-enantiomer, | 30.0 |
| D3-N-Deuterated) salt | |
| Amphetamine sulfate | 5.0 |
| Starch | 109.0 |
| Magnesium stearate | 1.0 |

In one non-limiting embodiment, a capsule, comprising deuterated R-Bk-5-MAPB, deuterated R-Bk-6-MAPB, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| Bk-5-MAPB (R-enantiomer, | 10.0 |
| D3-N-Deuterated) salt | |
| Bk-6-MAPB (R-enantiomer, | 30.0 |
| D3-N-Deuterated) salt | |
| Amphetamine sulfate | 5.0 |
| Starch | 109.0 |
| Magnesium stearate | 1.0 |

In one non-limiting embodiment, a capsule, comprising a deuterated compound of Formula E, a deuterated compound of Formula F, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| Compound of Formula E (R-enantiomer, | 10.0 |
| D3-N-Deuterated) salt | |
| Compound of Formula F (R-enantiomer, | 30.0 |
| D3-N-Deuterated) salt | |
| Amphetamine sulfate | 5.0 |
| Starch | 109.0 |
| Magnesium stearate | 1.0 |

In one non-limiting embodiment, a capsule, comprising deuterated R-6-MBPB and amphetamine sulfate, is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| 6-MBPB (R-enantiomer, | 40.0 |
| D3-N-Deuterated) salt | |
| Amphetamine sulfate | 5.0 |
| Starch | 109.0 |
| Magnesium stearate | 1.0 |

In one non-limiting embodiment, a capsule, comprising R-6-MAPB, S-6-MAPB, and psilocybin hydrochloride, is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| R-6-MAPB salt | 30.0 |
| S-6-MAPB salt | 10.0 |
| Psilocybin hydrochloride | 2.0 |
| Alpha lipoic acid | 40.0 |
| Starch | 72.0 |
| Magnesium stearate | 1.0 |

In one non-limiting embodiment, a capsule, comprising enantiomerically enriched 5-MBPB, enantiomerically enriched 6-MBPB, and psilocybin hydrochloride, is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| 5-MBPB (70% R-enantiomer) salt | 30.0 |
| 6-MBPB (70% S-enantiomer) salt | 10.0 |
| Psilocybin hydrochloride | 2.0 |
| Alpha lipoic acid | 40.0 |
| Starch | 72.0 |
| Magnesium stearate | 1.0 |

In one non-limiting embodiment, a capsule, comprising an enantiomerically enriched compound of Formula A, an enantiomerically enriched compound of Formula B, and psilocybin hydrochloride, is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| Compound of Formula A (70% R-enantiomer) | 30.0 |
| salt | |
| Compound of Formula B (70% S-enantiomer) | 10.0 |
| salt | |
| Psilocybin hydrochloride | 2.0 |
| Alpha lipoic acid | 40.0 |
| Starch | 72.0 |
| Magnesium stearate | 1.0 |

In one non-limiting embodiment, a capsule, comprising an enantiomerically enriched compound of Formula C, an enantiomerically enriched compound of Formula D, and psilocybin hydrochloride, is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| compound of Formula C (70% R-enantiomer) | 30.0 |
| salt | |
| compound of Formula D (70% S-enantiomer) | 10.0 |
| salt | |
| Psilocybin hydrochloride | 2.0 |
| Alpha lipoic acid | 40.0 |
| Starch | 72.0 |
| Magnesium stearate | 1.0 |

In one non-limiting embodiment, a capsule, comprising enantiomerically enriched Bk-5-MAPB, enantiomerically enriched Bk-6-MAPB, and psilocybin hydrochloride, is prepared using the ingredients below. The active ingredients, cellulose, starch, and magnesium stearate are blended, passed through a No. 20 mesh U.S. sieve, and filled into hard gelatin capsules in 155 mg 5 quantities.

| Ingredient | Quantity (mg/capsule) |
|---|---|
| Bk-5-MAPB (70% R-enantiomer) salt | 30.0 |
| Bk-6-MAPB (70% S-enantiomer) salt | 10.0 |
| Psilocybin hydrochloride | 2.0 |
| Alpha lipoic acid | 40.0 |
| Starch | 72.0 |
| Magnesium stearate | 1.0 |

In one non-limiting embodiment, a capsule, comprising an enantiomerically enriched compound of Formula E, an enantiomerically enriched compound of Formula F, and psilocybin hydrochloride, is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| Compound of Formula E (70% R-enantiomer) | 30.0 |
| salt | |
| Compound of Formula F (70% S-enantiomer) | 10.0 |
| salt | |
| Psilocybin hydrochloride | 2.0 |
| Alpha lipoic acid | 40.0 |
| Starch | 72.0 |
| Magnesium stearate | 1.0 |

It should be readily appreciated that the above formulation examples are illustrative only. Accordingly, it should be understood that reference to particular compounds(s) is likewise illustrative, and the compounds(s) in any of the non-limiting examples of combination formulations may be substituted by other compounds(s) of the invention. Likewise, any of the other active compounds (e.g., amphetamine sulfate or psilocybin hydrochloride as described above) may be substituted by a different other active compound, as may the inactive compounds.

Moreover, for any of S-5-MAPB, R-5-MAPB, S-6-MAPB, R-6-MAPB, 5-MBPB, 6-MBPB, Bk-5-MAPB, Bk-6-MAPB Formula A, Formula B, Formula C, Formula D, Formula E, and Formula F, or for any other active compounds of the invention, substitution of the compound by its prodrug, free base, salt, or hydrochloride salt shall be understood to provide merely an alternative embodiment still within the scope of the invention. Further, compositions within the scope of the invention should be understood to be open-ended and may include additional active or inactive compounds and ingredients.

The type of formulation employed for the administration of the compounds employed in the methods of the present invention generally may be dictated by the compound(s) employed, the type of pharmacokinetic profile desired from the route of administration and the compound(s), and the state of the patient.

### VIII. Additional Embodiments of the Present Invention

1. In certain embodiments a salt morphic form or morphic salt mixture of a benzofuran compound or an enantiomer or an enantiomerically enriched mixture of formula:

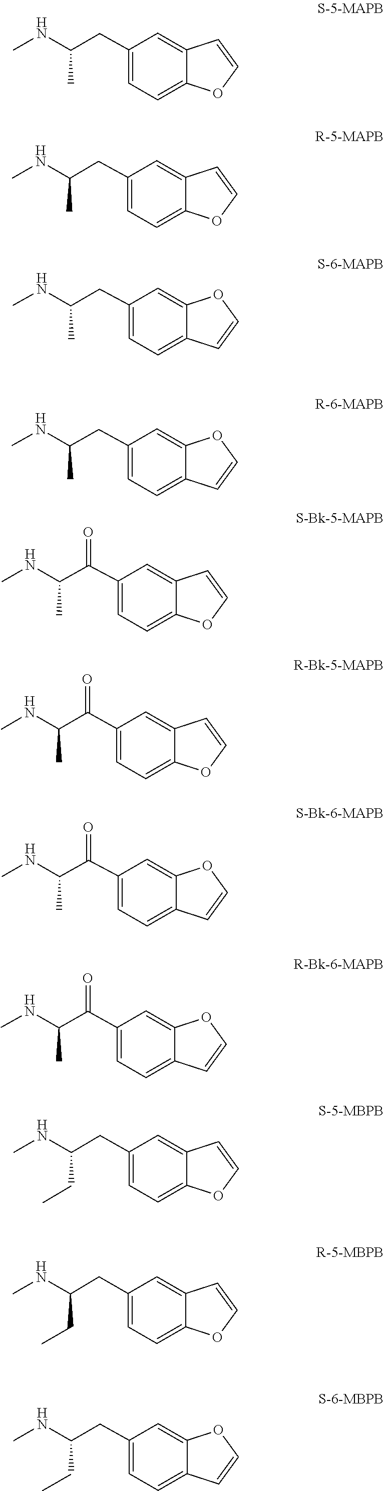

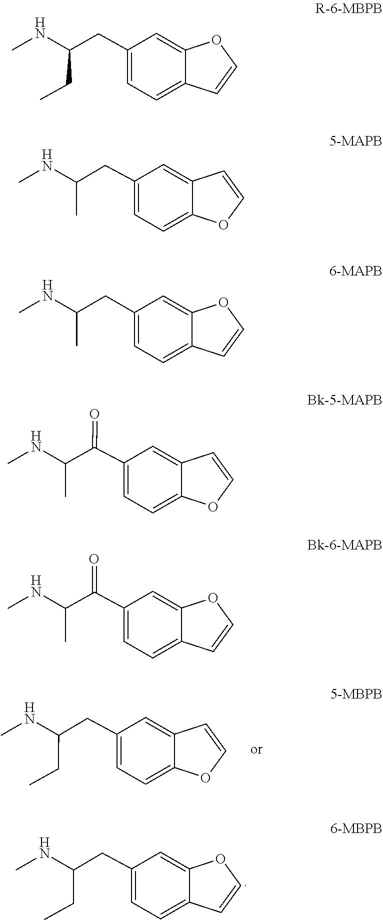

2. In certain embodiments salt morphic form or morphic salt mixture of a benzofuran compound or an enantiomer or an enantiomerically enriched mixture of formula:

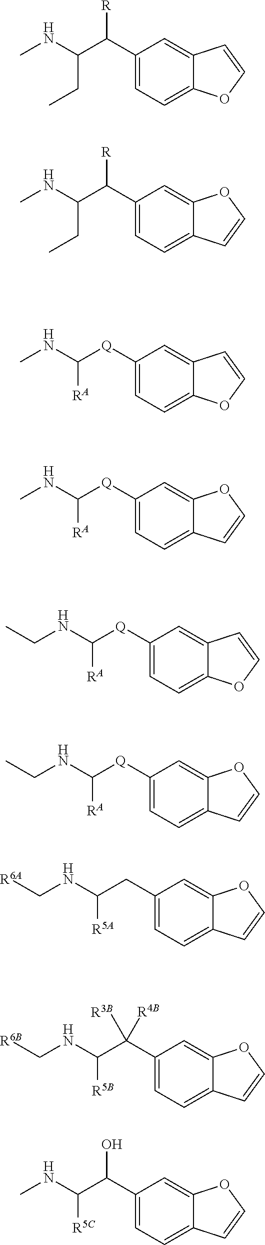

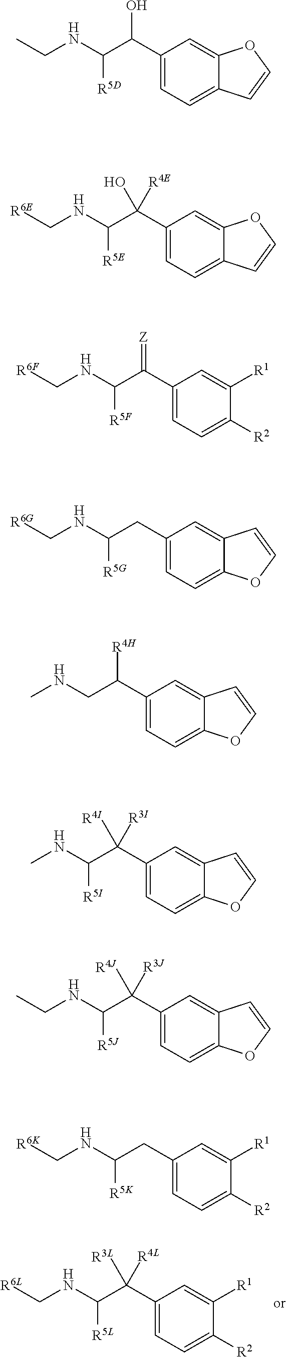

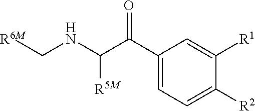

wherein:
   R is hydrogen or hydroxyl.
   R^{A} is —CH₃, —CH₂Y, —CHY₂, —CY₃, —CH₂CH₃, —CH₂CH₂Y, —CH₂CHY₂, —CH₂CY₃, —CH₂OH, or —CH₂CH₂OH;
   Q is selected from:

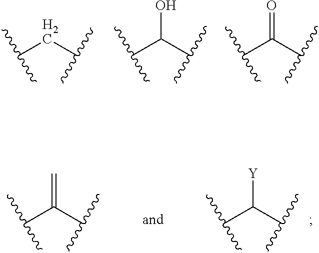

Y is halogen;
R¹ and R² are taken together as —OCH═CH— or —CH═CHO—;
R^{3B} and R^{4B} are independently selected from —H, —X, C₁-C₄ alkyl, —CH₂OH, —CH₂X, —CHX₂, and —CX₃, wherein at least one of R^{3B} and R^{4B} is not —H;
R^{3L} and R^{4L} are independently selected from —H, —X, —OH, C₁-C₄ alkyl, —CH₂OH, —CH₂X, —CHX₂, and —CX₃, wherein at least one of R^{3L} and R^{4L} is not —H;
R^{3I} and R^{4I} are independently selected from —H, —X, —OH, —CH₂OH, —CH₂X, —CHX₂, —CX₃, and C₁-C₄ alkyl; wherein at least one of R^{3I} and R^{4I} is not —H;
R^{3J} and R^{4J} are independently selected from —H, —X, —OH, C₁-C₄ alkyl, —CH₂OH, —CH₂X, —CHX₂, and —CX₃;
R^{4E} is selected from C₁-C₄ alkyl, —CH₂OH, —CH₂X, —CHX₂, and —CX₃;
R^{4H} is selected from —X, —CH₂CH₂CH₃, —CH₂OH, —CH₂X, and —CHX₂;
R^{5A} and R^{5G} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl, when R^{5A} is C₂ alkyl or H, R^{6A} is not —H, and when R^{5G} is —H or C₂ alkyl, R^{6G} is not —H;
R^{5B} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl;
R^{5C} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
R^{5D}, R^{5E}, R^{5F}, and R^{5J} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl, when R^{5F} is —H or C₁ alkyl, R^{6F} cannot be —H, and when R^{5J} is C₁ alkyl, at least one of R^{3J} and R^{4J} is not H; R^{5K} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
R^{5L} and R^{5M} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; and
R^{5I} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; wherein at least one of R^{3I}, R^{4I}, and R^{5I is not C}₁ alkyl;
R^{6A}, R^{6B}, R^{6E}, R^{6F}, and R^{6G} are independently selected from —H and —CH₃;
R^{6K}, R^{6L}, and R^{6M} are independently selected from —H and —CH₃;
X is independently selected from —F, —Cl, and —Br; and
Z is selected from O and CH₂.

3. The salt morphic form or morphic salt mixture of embodiment 1 or embodiment 2 comprising a benzofuran HCl salt.

4. The salt morphic form or morphic salt mixture of embodiment 1 comprising a racemic or enantioenriched 5-MAPB HCl Pattern 1A, wherein Pattern 1A is characterized by three or more peaks selected from 13.2, 15.1, 16.9, 17.9, 19.2, 20.8, 22.5, 24.4, 25.4, 25.8, 26.2, 27.6, and 31.6+/−0.4° 2theta.

5. The salt morphic form or morphic salt mixture of embodiment 4, wherein the XRPD pattern includes a peak at 19.2+/−0.4° 2theta.

6. The salt morphic form or morphic salt mixture of embodiment 4 or 5, wherein the XRPD pattern includes a peak at 27.6+/−0.4° 2theta.

7. The salt morphic form or morphic salt mixture of embodiment 4, 5, or 6, wherein the XRPD pattern includes a peak at 15.1+/−0.4° 2theta.

8. The salt morphic form or morphic salt mixture of embodiment 1 comprising S-5-MAPB HCl Pattern 1A, wherein Pattern 1A is characterized by three or more peaks selected from 6.7, 12.7, 13.4, 15.8, 19.0, 19.6, 21.2, 24.7, 25.1, 26.1, 26.8, 28.1, 29.0, 30.4, 31.1, and 39.7+/−0.4° 2theta.

9. The salt morphic form or morphic salt mixture of embodiment 8, wherein the XRPD pattern includes a peak at 26.8+/−0.4° 2theta.

10. The salt morphic form or morphic salt mixture of embodiment 8 or 9, wherein the XRPD pattern includes a peak at 19.0+/−0.4° 2theta.

11. The salt morphic form or morphic salt mixture of embodiment 8, 9, or 10, wherein the XRPD pattern includes a peak at 24.7+/−0.4° 2theta.

12. The salt morphic form or morphic salt mixture of embodiment 1 comprising S-6-MAPB HCl Pattern 1A, wherein Pattern 1A is characterized by three or more peaks selected from 17.7, 15.8, 21.3, 24.4, 25.6, 28.8, 31.7, and 35.0+/−0.4° 2theta.

13. The salt morphic form or morphic salt mixture of embodiment 12, wherein the XRPD pattern includes a peak at 17.7+/−0.4° 2theta.

14. The salt morphic form or morphic salt mixture of embodiment 12 or 13, wherein the XRPD pattern includes a peak at 21.3+/−0.4° 2theta.

15. The salt morphic form or morphic salt mixture of embodiment 12, 13, or 14, wherein the XRPD pattern includes a peak at 15.8+/−0.4° 2theta.

16. The salt morphic form or morphic salt mixture of any one of embodiments 1-15, comprising a HBr salt.

17. The salt morphic form or morphic salt mixture of embodiment 16 comprising a racemic or enantioenriched 5-MAPB HBr Pattern 2A, wherein Pattern 2A is characterized by three or more peaks selected from 11.9, 14.1, 16.2, 17.1, 22.2, 23.2, 23.7, 24.3, 26.9, 28.2, and 35.6+/−0.4° 2theta.

18. The salt morphic form or morphic salt mixture of embodiment 17, wherein the XRPD pattern includes a peak at 23.7+/−0.4° 2theta.

19. The salt morphic form or morphic salt mixture of embodiment 17 or 18, wherein the XRPD pattern includes a peak at 28.2+/−0.4° 2theta.

20. The salt morphic form or morphic salt mixture of embodiment 17, 18, or 19, wherein the XRPD pattern includes a peak at 16.2+/−0.4° 2theta.

21. The salt morphic form or morphic salt mixture of embodiment 16 comprising S-5-MAPB HBr Pattern 2A, wherein Pattern 2A is characterized by three or more peaks selected from 13.3, 19.0, 19.8, 20.3, 24.6, 26.0, 26.4, 27.2, 28.6, 30.1, 30.9, 33.1, and 35.3+/−0.4° 2theta.

22. The salt morphic form or morphic salt mixture of embodiment 21, wherein the XRPD pattern includes a peak at 26.4+/−0.4° 2theta.

23. The salt morphic form or morphic salt mixture of embodiment 21 or 22, wherein the XRPD pattern includes a peak at 26.0+/−0.4° 2theta.

24. The salt morphic form or morphic salt mixture of embodiment 21, 22, or 23, wherein the XRPD pattern includes a peak at 13.3+/−0.4° 2theta.

25. The salt morphic form or morphic salt mixture of embodiment 16 comprising S-6-MAPB HBr Pattern 2A, wherein Pattern 2A is characterized by three or more peaks selected from 16.3, 17.7, 18.2, 21.1, 21.4, 22.0, 22.6, 24.1, 25.2, 26.6, 27.1, 28.2, 28.5, 28.8, 29.2, 30.0, 30.5, 31.2, 31.3, 32.2, 32.4, 33.0, 33.5, 33.9, 35.1, 36.2, 38.0, 38.6, and 38.8+/−0.4° 2theta.

26. The salt morphic form or morphic salt mixture of embodiment 25, wherein the XRPD pattern includes a peak at 21.4+/−0.4° 2theta.

27. The salt morphic form or morphic salt mixture of embodiment 25 or 26, wherein the XRPD pattern includes a peak at 25.2+/−0.4° 2theta.

28. The salt morphic form or morphic salt mixture of embodiment 25, 26, or 27, wherein the XRPD pattern includes a peak at 32.2+/−0.4° 2theta.

29. The salt morphic form or morphic salt mixture of any one of embodiments 1-28, comprising a H₂SO₄ salt.

30. The salt morphic form or morphic salt mixture of any one of embodiments 1-29, comprising a H₃PO₄ salt.

31. The salt morphic form or morphic salt mixture of embodiment 30 comprising a racemic or enantioenriched 5-MAPB H₃PO₄ Pattern 4A, wherein Pattern 4A is characterized by three or more peaks selected from 13.3, 13.6, 17.7, 18.1, 19.5, 20.1, 21.6, 22.3, 24.1, 25.2, 26.0, 26.9, 27.8, 30.4, 34.7, and 37.7+/−0.4° 2theta.

32. The salt morphic form or morphic salt mixture of embodiment 30 comprising a racemic or enantioenriched 5-MAPB H₃PO₄ Pattern 4B, wherein Pattern 4B is characterized by three or more peaks selected from 12.3, 13.8, 17.0, 18.7, 20.9, 21.8, 23.4, 24.5, 27.1, and 28.2+/−0.4° 2theta.

33. The salt morphic form or morphic salt mixture of embodiment 30 comprising a racemic or enantioenriched 5-MAPB H₃PO₄ Pattern 4C, wherein Pattern 4C is characterized by three or more peaks selected from 12.9, 14.5, 16.3, 17.6, 18.1, 21.3, 22.0, 24.7, 25.5, 25.9, 26.6, 27.4, 28.5, 29.3, 30.6, and 35.7+/−0.4° 2theta.

34. The salt morphic form or morphic salt mixture of embodiment 30 comprising S-5-MAPB H₃PO₄ Pattern 4A, wherein Pattern 4A is characterized by three or more peaks selected from 13.3, 16.4, 17.5, 19.2, 20.0, 21.9, 22.6, 23.9, 24.9, 26.1, and 27.3+/−0.4° 2theta.

35. The salt morphic form or morphic salt mixture of embodiment 34, wherein the XRPD pattern includes a peak at 13.3+/−0.4° 2theta.

36. The salt morphic form or morphic salt mixture of embodiment 34 or 35, wherein the XRPD pattern includes a peak at 21.9+/−0.4° 2theta.

37. The salt morphic form or morphic salt mixture of embodiment 34, 35, or 36, wherein the XRPD pattern includes a peak at 17.5+/−0.4° 2theta.

38. The salt morphic form or morphic salt mixture of embodiment 30 comprising S-6-MAPB H₃PO₄ Pattern 3A, wherein Pattern 3A is characterized by three or more peaks selected from 13.5, 15.1, 17.0, 17.8, 18.4, 19.3, 19.8, 20.1, 20.6, 21.5, 22.2, 22.6, 24.5, 25.6, 26.6, 26.8, 27.2, 27.6, 29.5, 32.9, 35.1, 35.3, 37.8, and 39.6+/−0.4° 2theta.

39. The salt morphic form or morphic salt mixture of embodiment 38, wherein the XRPD pattern includes a peak at 22.2+/−0.4° 2theta.

40. The salt morphic form or morphic salt mixture of embodiment 38 or 39, wherein the XRPD pattern includes a peak at 13.5+/−0.4° 2theta.

41. The salt morphic form or morphic salt mixture of embodiment 30 comprising S-6-MAPB H₃PO₄ Pattern 3B, wherein Pattern 3B is characterized by three or more peaks selected from 10.8, 13.1, 16.5, 17.3, 17.6, 18.6, 18.7, 19.6, 21.2, 21.6, 22.1, 22.7, 24.6, 25.4, 25.5, 26.0, 26.1, 26.6, 26.7, 26.8, 27.3, 27.9, 28.4, 28.7, 29.2, 29.4, 30.0, 30.2, 30.7, 31.2, 32.6, 32.7, 34.0, 34.4, 34.6, 34.7, 35.5, 35.9, 36.0, 36.8, 37.6, 39.4, and 39.7+/−0.4° 2theta.

42. The salt morphic form or morphic salt mixture of any one of embodiments 1-41, comprising a HNO₃ salt.

43. The salt morphic form or morphic salt mixture of any one of embodiments 1-42, comprising a methanesulfonic salt.

44. The salt morphic form or morphic salt mixture of any one of embodiments 1-43, comprising a succinic salt.

45. The salt morphic form or morphic salt mixture of any one of embodiments 1-44, comprising an oxalic salt.

46. The salt morphic form or morphic salt mixture of embodiment 45 comprising a racemic or enantioenriched 5-MAPB oxalic Pattern 9A, wherein Pattern 9A is characterized by three or more peaks selected from 10.6, 13.2, 14.1, 18.9, 19.9, 21.0, 22.2, 22.9, 23.8, 24.7, 25.7, 26.4, 28.2, 31.1, 33.0, 35.3, 36.8, and 37.9+/−0.4° 2theta.

47. The salt morphic form or morphic salt mixture of embodiment 46, wherein the XRPD pattern includes a peak at 19.9+/−0.4° 2theta.

48. The salt morphic form or morphic salt mixture of embodiment 46 or 47, wherein the XRPD pattern includes a peak at 22.2+/−0.4° 2theta.

49. The salt morphic form or morphic salt mixture of embodiment 46, 47, or 48, wherein the XRPD pattern includes a peak at 25.7+/−0.4° 2theta.

50. The salt morphic form or morphic salt mixture of embodiment 45 comprising S-5-MAPB oxalic Pattern 8A, wherein Pattern 8A is characterized by three or more peaks selected from 10.6, 12.9, 14.0, 18.9, 20.3, 20.6, 21.2, 21.8, 22.5, 24.8, 25.9, 26.5, 27.8, 30.4, 30.9, 32.4, 33.2, 34.7, 35.7, and 37.1+/−0.4° 2theta.

51. The salt morphic form or morphic salt mixture of embodiment 50, wherein the XRPD pattern includes a peak at 22.5+/−0.4° 2theta.

52. The salt morphic form or morphic salt mixture of embodiment 50 or 51, wherein the XRPD pattern includes a peak at 25.9+/−0.4° 2theta.

53. The salt morphic form or morphic salt mixture of embodiment 50, 51, or 52, wherein the XRPD pattern includes a peak at 20.6+/−0.4° 2theta.

54. The salt morphic form or morphic salt mixture of embodiment 45 comprising S-6-MAPB oxalic Pattern 5A, wherein Pattern 5A is characterized by three or more peaks selected from 10.3, 12.4, 12.8, 17.0, 18.8, 19.7, 20.5, 21.1, 21.9, 22.3, 23.1, 23.5, 24.9, 25.5, 25.9, 26.4, 27.0, 28.2, 29.1, 29.5, 30.1, 32.0, 32.3, 34.2, 34.8, 35.8, 37.2, and 39.0+/−0.4° 2theta.

55. The salt morphic form or morphic salt mixture of embodiment 54, wherein the XRPD pattern includes a peak at 37.2+/−0.4° 2theta.

56. The salt morphic form or morphic salt mixture of embodiment 54 or 55, wherein the XRPD pattern includes a peak at 24.9+/−0.4° 2theta.

57. The salt morphic form or morphic salt mixture of embodiment 54, 55, or 56, wherein the XRPD pattern includes a peak at 20.5+/−0.4° 2theta.

58. The salt morphic form or morphic salt mixture of any one of embodiments 1-57, comprising a maleic salt.

59. The salt morphic form or morphic salt mixture of embodiment 58 comprising a racemic or enantioenriched 5-MAPB maleic Pattern 10A, wherein Pattern 10A is characterized by three or more peaks selected from 16.1, 17.5, 17.7, 18.7, 19.3, 19.7, 21.7, 22.5, 22.8, 23.4, 23.5, 24.8, 26.1, and 29.4+/−0.4° 2theta.

60. The salt morphic form or morphic salt mixture of embodiment 59, wherein the XRPD pattern includes a peak at 23.5+/−0.4° 2theta.

61. The salt morphic form or morphic salt mixture of embodiment 59 or 60, wherein the XRPD pattern includes a peak at 23.4+/−0.4° 2theta.

62. The salt morphic form or morphic salt mixture of embodiment 59, 60, or 61, wherein the XRPD pattern includes a peak at 17.7+/−0.4° 2theta.

63. The salt morphic form or morphic salt mixture of any one of embodiments 1-62, comprising a fumaric salt.

64. The salt morphic form or morphic salt mixture of embodiment 63 comprising S-5-MAPB fumaric Pattern 10A, wherein Pattern 10A is characterized by three or more peaks selected from 8.5, 16.0, 17.6, 18.1, 20.1, 20.9, 21.7, 23.1, 23.6, 24.0, 25.2, 26.2, 28.5, 29.5, 30.4, and 30.7+/−0.4° 2theta.

65. The salt morphic form or morphic salt mixture of embodiment 64, wherein the XRPD pattern includes a peak at 23.6+/−0.4° 2theta.

66. The salt morphic form or morphic salt mixture of embodiment 64 or 65, wherein the XRPD pattern includes a peak at 18.1+/−0.4° 2theta.

67. The salt morphic form or morphic salt mixture of embodiment 64, 65, or 66, wherein the XRPD pattern includes a peak at 17.6+/−0.4° 2theta.

68. The salt morphic form or morphic salt mixture of any one of embodiments 1-67, comprising a saccharate salt.

69. The salt morphic form or morphic salt mixture of any one of embodiments 1-68, comprising an aspartate salt.

70. The salt morphic form or morphic salt mixture of any one of embodiments 1-69, comprising a L-Arginine salt.

71. The salt morphic form or morphic salt mixture of any one of embodiments 1-70, comprising a L-Lysine salt.

72. The salt morphic form or morphic salt mixture of any one of embodiments 1-71, comprising a salt selected from 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, finnarate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, sethiona, hybenzate, hydrabamine, hydrobromide, hydrobromide/bromide, hydrochloride, hydroiodide, hydroxide, hydroxybenzoate, hydroxynaphthoate, iodide, isethionate, sethionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, and xinafoate.

73. The salt morphic form or morphic salt mixture of any one of embodiments 1-72, wherein there is only one salt present.

74. The salt morphic form or morphic salt mixture of any one of embodiments 1-72, wherein there are two salts present.

75. The salt morphic form or morphic salt mixture of any one of embodiments 1-72, wherein there are three salts present.

76. The salt morphic form or morphic salt mixture of any one of embodiments 1-72, wherein there are four salts present.

77. The salt morphic form or morphic salt mixture of any one of embodiments 1-76, wherein the benzofuran compound is racemic.

78. The salt morphic form or morphic salt mixture of any one of embodiments 1-76, wherein the benzofuran compound is enantiomerically enriched as an R-enantiomer.

79. The salt morphic form or morphic salt mixture of any one of embodiments 1-76, wherein the benzofuran compound is enantiomerically enriched as an S-enantiomer.

80. The salt morphic form or morphic salt mixture of any one of embodiments 1-76, wherein the benzofuran compound is a substantially pure R-enantiomer.

81. The salt morphic form or morphic salt mixture of any one of embodiments 1-76, wherein the benzofuran compound is a substantially pure S-enantiomer.

82. The salt morphic form or morphic salt mixture of any one of embodiments 1-81, further comprising an additional benzofuran compound described in embodiment 1 or embodiment 2.

83. The salt morphic form or morphic salt mixture of embodiment 82 wherein the additional benzofuran compound is a racemate.

84. The salt morphic form or morphic salt mixture of embodiment 82 wherein the additional benzofuran compound is enantiomerically enriched as an R-enantiomer.

85. The salt morphic form or morphic salt mixture of embodiment 82 wherein the additional benzofuran compound is enantiomerically enriched as an S-enantiomer.

86. The salt morphic form or morphic salt mixture of embodiment 82 wherein the additional benzofuran compound is a substantially pure R-enantiomer.

87. The salt morphic form or morphic salt mixture of embodiment 82 wherein the additional benzofuran compound is a substantially pure S-enantiomer.

88. The salt morphic form or morphic salt mixture of any one of embodiments 1-87, wherein the compound or mixture of compounds is selected from 5-MAPB, 6-MAPB, 5-MBPB, 6-MBPB, Bk-5-MAPB, Bk-6-MAPB, Bk-5-MBPB and Bk-6-MBPB.

89. The salt morphic form or morphic salt mixture of any one of embodiments 1-88, wherein the salt morphic form or morphic salt mixture has entactogenic properties.

90. The salt morphic form or morphic salt mixture of any one of embodiments 1-89, wherein the salt morphic form or morphic salt mixture has serotonin-receptor-dependent properties.

91. The salt morphic form or morphic salt mixture of any one of embodiments 1-90, wherein the salt morphic form or morphic salt mixture has decreased hallucinogenic effects relative to MDMA.

92. The salt morphic form or morphic salt mixture of any one of embodiments 1-91, wherein the salt morphic form or morphic salt mixture has decreased unwanted psychoactive effects relative to MDMA.

93. The salt morphic form or morphic salt mixture of any one of embodiments 1-92, wherein the salt morphic form or morphic salt mixture has decreased physiological effects relative to MDMA.

94. The salt morphic form or morphic salt mixture of any one of embodiments 1-93, wherein the salt morphic form or morphic salt mixture has decreased abuse potential relative to MDMA.

95. The salt morphic form or morphic salt mixture of any one of embodiments 1-94, wherein the salt morphic form or morphic salt mixture has decreased hallucinogenic effects relative to a clinically used 5-HT_{2A} agonist.

96. The salt morphic form or morphic salt mixture of any one of embodiments 1-95, wherein the salt morphic form or morphic salt mixture has decreased unwanted psychoactive effects relative to a clinically used 5-HT_{2A} agonist.

97. The salt morphic form or morphic salt mixture of any one of embodiments 1-96, wherein the salt morphic form or morphic salt mixture has decreased physiological effects relative to a clinically used 5-HT_{2A} agonist.

98. The salt morphic form or morphic salt mixture of any one of embodiments 1-97, wherein the salt morphic form or morphic salt mixture that shows the therapeutic effect of emotional openness.

99. The salt morphic form or morphic salt mixture of any one of embodiments 1-98, wherein the benzofuran compound is:

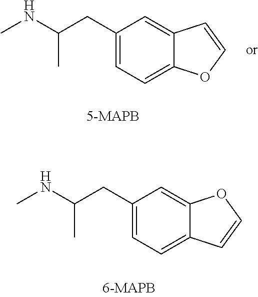

or an enantiomerically enriched mixture thereof.

100. The salt morphic form or morphic salt mixture of any one of embodiments 1-98,
wherein the benzofuran compound is:

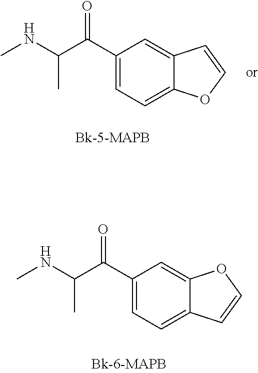

or an enantiomerically enriched mixture thereof.

101. The salt morphic form or morphic salt mixture of any one of embodiments 1-98,
wherein the benzofuran compound is:

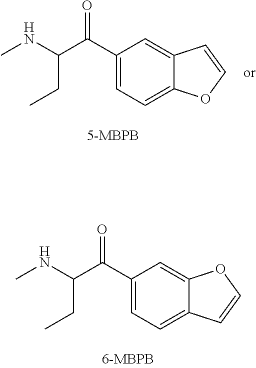 [SMILES: CCC(NC)C(=O)c1ccc2occc2c1 ; CCC(NC)C(=O)c1ccc2ccoc2c1]

or an enantiomerically enriched mixture thereof.

102. The salt morphic form or morphic salt mixture of any one of embodiments 1-98,
wherein the benzofuran compound is:

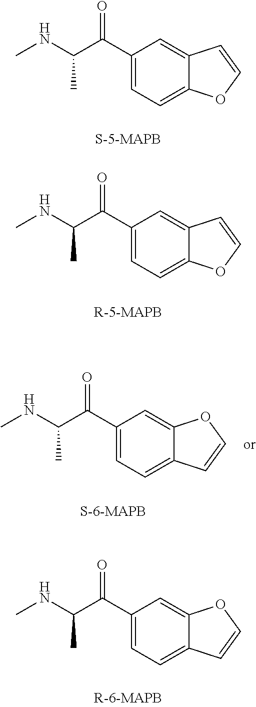

103. The salt morphic form or morphic salt mixture of any one of embodiments 1-98,
wherein the benzofuran compound is:

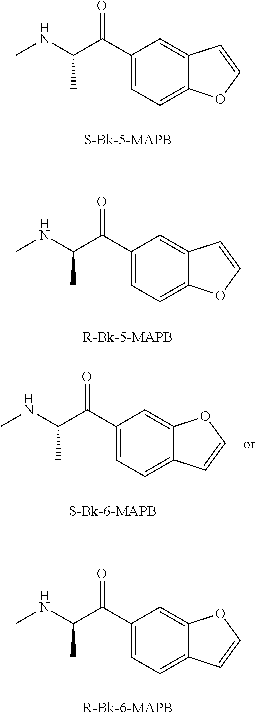

104. The salt morphic form or morphic salt mixture of any one of embodiments 1-98,
wherein the benzofuran compound is:

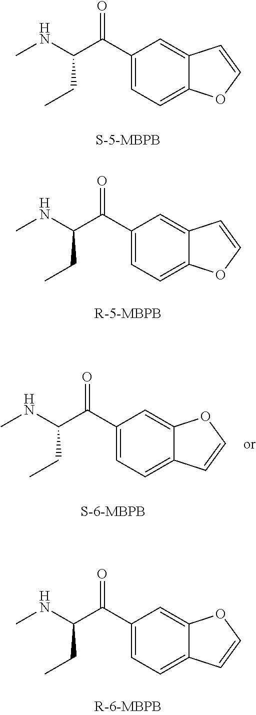 [SMILES: CC[C@H](NC)C(=O)c1ccc2occc2c1 ; CC[C@@H](NC)C(=O)c1ccc2occc2c1 ; CC[C@H](NC)C(=O)c1ccc2ccoc2c1 ; CC[C@@H](NC)C(=O)c1ccc2ccoc2c1]

105. In certain embodiments a pharmaceutical composition comprising a salt morphic form or morphic salt mixture of any one of embodiments 1-104 and a pharmaceutically acceptable excipient.

106. In certain embodiments a pharmaceutical composition prepared from a salt morphic form or morphic salt mixture of any one of embodiments 1-104.

107. The pharmaceutical composition of embodiment 105 or 106 wherein the composition is administered systemically.

108. The pharmaceutical composition of embodiment 105 or 106 wherein the composition is administered orally.

109. The pharmaceutical composition of embodiment 105 or 106 wherein the composition is administered to mucosal tissue.

110. The pharmaceutical composition of embodiment 105 or 106 wherein the composition is administered rectally.

111. The pharmaceutical composition of embodiment 105 or 106 wherein the composition is administered topically.

112. The pharmaceutical composition of embodiment 105 or 106 wherein the composition is administered subcutaneously.

113. The pharmaceutical composition of embodiment 105 or 106 wherein the composition is administered intravenously.

114. The pharmaceutical composition of embodiment 105 or 106 wherein the composition is administered intramuscularly.

115. The pharmaceutical composition of embodiment 105 or 106 wherein the composition is administered via inhalation.

116. The pharmaceutical composition of embodiment 108 wherein the composition is administered as a tablet.

117. The pharmaceutical composition of embodiment 108 wherein the composition is administered as a gelcap.

118. The pharmaceutical composition of embodiment 108 wherein the composition is administered as a capsule.

119. The pharmaceutical composition of embodiment 108 wherein the composition is administered as an aqueous emulsion.

120. The pharmaceutical composition of embodiment 108 wherein the composition is administered as an aqueous solution.

121. The pharmaceutical composition of embodiment 108 wherein the composition is administered as a pill.

122. The pharmaceutical composition of embodiment 108 wherein the composition is administered as a buccal tablet.

123. The pharmaceutical composition of embodiment 108 wherein the composition is administered as a sublingual tablet.

124. The pharmaceutical composition of embodiment 108 wherein the composition is administered as a sublingual strip.

125. The pharmaceutical composition of embodiment 109 wherein the composition is administered as a sublingual liquid.

126. The pharmaceutical composition of embodiment 109 wherein the composition is administered as a sublingual spray.

127. The pharmaceutical composition of embodiment 109 wherein the composition is administered as a sublingual gel.

128. The pharmaceutical composition of embodiment 111 wherein the composition is administered as a cream.

129. The pharmaceutical composition of embodiment 111 wherein the composition is administered as a topical solution.

130. The pharmaceutical composition of embodiment 113 wherein the composition is administered as an aqueous solution.

131. The pharmaceutical composition of embodiment 115 wherein the composition is administered as a powder.

132. The pharmaceutical composition of embodiment 115 wherein the composition is administered as an aerosol.

133. In certain embodiments a morphic form selected from Pattern 1A RS-5-MAPB HCl, Pattern 2A RS-5-MAPB HBr, Pattern 4A RS-5-MAPB H₃PO₄, Pattern 4B RS-5-MAPB H₃PO₄, Pattern 9A RS-5-MAPB oxalic acid, Pattern 10A RS-5-MAPB maleic acid, Pattern 1A S-5-MAPB HCl, Pattern 2A S-5-MAPB HBr, Pattern 4A S-5-MAPB H₃PO₄, Pattern 8A S-5-MAPB oxalic acid, Pattern 10A S-5-MAPB fumaric acid, Pattern 1A R-5-MAPB HCl, Pattern 1A S-6-MAPB HCl, Pattern 2A S-6-MAPB HBr, Pattern 3A S-6-MAPB H₃PO₄, and Pattern 5A S-6-MAPB oxalic acid, Pattern 1A S-BK-5-MAPB HCl, Pattern 1B S-BK-5-MAPB HCl, S-BK-5-MAPB HBr, Pattern 3A S-BK-5-MAPB H₂SO₄, S-BK-S-MAPB H₃PO₄, S-BK-5-MAPB HNO₃, S-BK-5-MAPB methane sulfonic acid, S-BK-5-MAPB tartaric acid, S-BK-5-MAPB succinic acid, Pattern 9A S-BK-5-MAPB oxalic acid, Pattern 10A S-BK-5-MAPB maleic acid, Pattern 11A S-BK-5-MAPB malic acid, S-BK-5-MAPB citric acid, Pattern 13A S-BK-5-MAPB fumaric acid, Pattern 14A S-BK-5-MAPB benzoic acid, Pattern 15A S-BK-5-MAPB salicylic acid, Pattern 15B S-BK-S-MAPB salicylic acid, Pattern 1A S-6-MBPB HCl, Pattern 2A S-6-MBPB HBr, S-6-MBPB H₂SO₄, Pattern 4A S-6-MBPB H₃PO₄, Pattern 5A S-6-MBPB HNO₃, S-6-MBPB methane sulfonic acid, Pattern 7A S-6-MBPB tartaric acid, Pattern 8A S-6-MBPB succinic acid, Pattern 9A S-6-MBPB oxalic acid, Pattern 10A S-6-MBPB maleic acid, S-6-MBPB malic acid, Pattern 12A S-6-MBPB citric acid, Pattern 13A S-6-MBPB fumaric acid, Pattern 13B S-6-MBPB fumaric acid, S-6-MBPB benzoic acid, S-6-MBPB salicylic acid, Pattern 1A S-5-MBPB HCl, Pattern 2B S-S-MBPB HBr, Pattern 3A S-5-MBPB H₃PO₄, S-5-MBPB HNO₃, S-5-MBPB tartaric acid, Pattern 6A S-S-MBPB succinic acid, S-5-MBPB B oxalic acid, Pattern 8A S-S-MBPB maleic acid, Pattern 9A S-5-MBPB citric acid, Pattern 10A S-5-MBPB fumaric acid, Pattern 1A R-S-MBPB HCl, Pattern 3A R-S-MBPB H₃PO₄, Pattern 8A R-5-MBPB maleic acid, R-5-MBPB fumaric acid, Pattern 1A R-6-MBPB HCl, Pattern 2A R-6-MBPB HBr, and Pattern 9A R-6-MBPB oxalate.

134. In certain embodiments a method for treating a central nervous system disorder comprising administering an effective amount of a salt morphic form, morphic salt mixture, or pharmaceutical composition of any one of embodiments 1-133 to a host in need thereof.

135. The method of embodiment 134 wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism and dissociative disorders.

136. The method of embodiment 134 or 135 wherein the host is a human.

137. The method of any one of embodiments 134-136 wherein the central nervous system disorder is post-traumatic stress disorder.

138. The method of any one of embodiments 134-136 wherein the central nervous system disorder is adjustment disorder.

139. The method of any one of embodiments 134-136 wherein the central nervous system disorder is generalized anxiety.

140. The method of any one of embodiments 134-136 wherein the central nervous system disorder is social anxiety.

141. The method of any one of embodiments 134-136 wherein the central nervous system disorder is depression.

142. The method of any one of embodiments 134-136 wherein the central nervous system disorder is a substance use disorder.

143. The method of any one of embodiments 134-136 wherein the central nervous system disorder is an attachment disorder.

144. The method of any one of embodiments 134-136 wherein the central nervous system disorder is schizophrenia.

145. The method of any one of embodiments 134-136 wherein the central nervous system disorder is an eating disorder.

146. The method of embodiment 145 wherein the eating disorder is bulimia.

147. The method of embodiment 145 wherein the eating disorder is binge eating.

148. The method of embodiment 145 wherein the eating disorder is anorexia.

149. The method of any one of embodiments 134-136 wherein there are multiple central nervous system disorders.

150. The method of any one of embodiments 134-136 wherein the central nervous system disorder is a neurological disorder.

151. The method of embodiment 150 wherein the neurological disorder is stroke.

152. The method of embodiment 150 wherein the neurological disorder is brain trauma.

153. The method of embodiment 150 wherein the neurological disorder is dementia.

154. The method of embodiment 150 wherein the neurological disorder is a neurodegenerative disease or disorder.

155. The method of embodiment 154 wherein the neurodegenerative disease or disorder is selected from: Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.

156. The method of any one of embodiments 134-155 wherein the salt morphic form or morphic salt mixture is administered in a clinical setting.

157. The method of any one of embodiments 134-155 wherein the salt morphic form or morphic salt mixture is administered in an at-home setting.

158. The method of any one of embodiments 134-155 wherein the salt morphic form or morphic salt mixture is administered during a psychotherapy session.

159. The method of any one of embodiments 134-155 wherein the salt morphic form or morphic salt mixture is administered during a counseling session.

160. In certain embodiments a salt morphic form, morphic salt mixture, or pharmaceutical composition of any one of embodiments 1-133 for use in the treatment of a central nervous system disorder in a host.

161. Use of a salt morphic form, morphic salt mixture, or pharmaceutical composition of any one of embodiments 1-133 in the treatment of a central nervous system disorder in a host.

162. Use of a salt morphic form, morphic salt mixture, or pharmaceutical composition of any one of embodiments 1-133 in the manufacture of a medicament for the treatment of a central nervous system disorder in a host.

163. Pharmaceutical composition comprising a salt morphic form, morphic salt mixture, or pharmaceutical composition of any one of embodiments 1-133 for use in the treatment of a central nervous system disorder in a host.

164. In certain embodiments a racemic, enantiomerically pure, or an enantiomerically enriched mixture of the S-enantiomer and R-enantiomer of 6-MBPB:

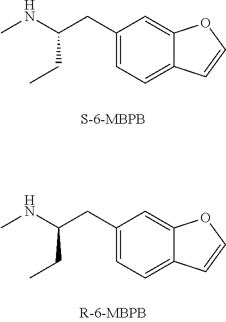

or a pharmaceutically acceptable salt or mixed salt thereof.

165. In certain embodiments a method for treating a central nervous system disorder selected from: depression, dysthymia, anxiety, generalized anxiety, social anxiety, panic, adjustment disorders, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism and dissociative disorders in a host in need thereof comprising administering 6-MBPB or a pharmaceutically acceptable salt or mixed salt thereof.

166. In certain embodiments a use of 6-MBPB or a pharmaceutically acceptable salt thereof in the manufacture of a medicament for the treatment of a central nervous system disorder selected from: depression, dysthymia, anxiety, generalized anxiety, social anxiety, panic, adjustment disorders, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism and dissociative disorders in a host.

167. In certain embodiments a pharmaceutical composition comprising 6-MBPB or a pharmaceutically acceptable salt or mixed salt thereof for use in the treatment of a central nervous system disorder selected from: depression, dysthymia, anxiety, generalized anxiety, social anxiety, panic, adjustment disorders, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism and dissociative disorders in a host is provided.

### IX. Dosage Regimes

The salts, salt mixtures, and morphic forms of compounds or pharmaceutically acceptable formulations of the present invention can be administered to the host in any amount, and with any frequency, that achieves the goals of the invention as used by the healthcare provider, or otherwise by the host in need thereof, typically a human, as necessary or desired.

In certain embodiments, the composition as described herein is provided only in a controlled counseling session, and administered only once, or perhaps 2, 3, 4, or 5 or more times in repeated counseling sessions to address a mental disorder as described herein.

In other embodiments, the composition as described herein is provided outside of a controlled counseling session, and perhaps self-administered, as needed to perhaps 2, 3, 4, or 5 or more times in to address a mental disorder as described herein.

In other embodiments, the composition of the present invention may be administered on a routine basis for mental wellbeing or for entactogenic treatment.

The compounds of the current invention can be administered in a variety of doses, routes of administration, and dosing regimens, based on the indication and needs of the patient. Non-limiting examples of therapeutic use include discrete psychotherapeutic sessions, ad libitum use for treatment of episodic disorders, and ongoing use for treatment of subchronic and chronic disorders.

### Psychotherapeutic Sessions

For some indications, the medicine is taken in discrete psychotherapy or other beneficial sessions. It is anticipated that these sessions will typically be separated by more than 5 half-lives of the medicine and, for most patients, will typically occur only 1 to 5 times each year.

For these sessions, it will typically be desirable to induce clearly perceptible entactogenic effects that will facilitate fast therapeutic progress. Non-exhaustive examples of oral doses of medicine that produce clearly perceptible entactogenic effects include: about 40 to about 120 mg of non-racemic 5-MAPB, about 40 to about 120 mg of non-racemic 6-MAPB, about 25 to about 300 mg of 5-MBPB, about 25 to about 300 mg of 6-MBPB, about 75 to about 500 mg of BK-5-MAPB, about 75 to about 500 mg of BK-6-MAPB, about 75 to about 800 mg of BK-5-MBPB, about 75 to about 800 mg of BK-6-MBPB.

It is anticipated that the medicine would be taken once or, more rarely, two or three times in a single therapeutic session. In these cases, it is common for each subsequent dose to be half of the previous dose or lower. Multiple doses within a session typically occur because either the patient's sensitivity to the medicine was unknown and too low of an initial dose was employed or because the patient is experiencing a productive session and it is desirable to extend the duration of therapeutic effects. Controlled release preparations may be used to lengthen the duration of therapeutic effects from a single administration of the medicine. In cases where multiple administrations are used in a session, it is anticipated that individual doses will be lower so that plasma concentrations remain within a desired therapeutic range.

Non-limiting, non-exhaustive examples of indications that may benefit from psychotherapeutic sessions include depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders. Also included as exemplary situations where an individual would benefit from a psychotherapeutic session are situations from a reduction of neuroticism or psychological defensiveness, an increase in openness to experience, an increase in creativity, or an increase in decision-making ability.

### Ad Libitum Use for Treatment of Episodic Disorders

For some indications, such as social anxiety, where the patient has need for relief from episodic occurrence of a disorder, it is anticipated that the medicine would be taken as needed but that uses should be separated by more than 5 half-lifes of the medicine to avoid bioaccumulation and formation of tolerance.

For treating episodic disorders, clearly perceptible entactogenic effects are often not desirable, as they can impair some aspects of functioning. Non-exhaustive examples of oral doses of medicine that produce subtle, barely perceptible therapeutic effects include: about 10 to about 60 mg of non-racemic 5-MAPB, about 10 to about 60 mg of non-racemic 6-MAPB, about 10 to about 35 mg of 5-MBPB, about 10 to about 35 mg of 6-MBPB, about 20 to about 150 mg of BK-5-MAPB, about 20 to about 150 mg of BK-6-MAPB, about 20 to about 200 mg of BK-5-MBPB, and about 20 to about 200 mg of BK-6-MBPB.

Non-limiting, non-exhaustive examples of indications that may benefit from episodic treatment are the same as those listed in the previous section provided that clinically significant signs and symptoms worsen episodically or in predictable contexts.

### Ongoing Use for Treatment of Subchronic and Chronic Disorders

For some indications, such as substance use disorders, inflammatory conditions, and neurological indications, including treatment of stroke, brain trauma, dementia, and neurodegenerative diseases, where the patient has need for ongoing treatment, it is anticipated that the medicine would be taken daily, twice daily, or three times per day. With some indications (subchronic disorders), such as treatment of stroke or traumatic brain injury, it is anticipated that treatment duration will be time-limited and dosing will be tapered when the patient has recovered. An example dose taper regimen is a reduction in dose of 10% of the original dose per week for nine weeks. With other, chronic disorders, such as dementia, it is anticipated that treatment will be continued as long as the patient continues to receive clinically significant benefits.

For treating subchronic and chronic disorders, clearly perceptible entactogenic effects are often not desirable. Non-exhaustive examples of oral doses of medicine that produce subtle, barely perceptible therapeutic effects with ongoing dosing include: about 5 to about 60 mg of non-racemic 5-MAPB, about 5 to about 60 mg of non-racemic 6-MAPB, about 5 to about 30 mg of 5-MBPB, about 5 to about 30 mg of 6-MBPB, about 10 to about 150 mg of BK-5-MAPB, about 10 to about 150 mg of BK-6-MAPB, about 10 to about 200 mg of BK-5-MBPB, and about 10 to about 200 mg of BK-6-MBPB.

Non-limiting, non-exhaustive examples of subchronic and chronic disorders that may benefit from regular treatment include migraine, headaches (e.g., cluster headache), neurodegenerative disorders, Alzheimer's disease, Parkinson's disease, schizophrenia, stroke, traumatic brain injury, phantom limb syndrome, and other conditions where increasing neuronal plasticity is desirable.

### X. EXAMPLES

### EXAMPLE 1: Production of Enantiomerically Enriched Preparations

Racemic 5-MAPB HCl (not less than 99.9% pure) was purchased (Chemical Collective, Netherlands). Enantiomeric enrichment of 2 g of 5-MAPB HCl was performed using supercritical fluid chromatography (SFC), with details listed below:

### Preparative SFC Method

Column: 2.1×25.0 cm Chiralpak AD-H (Chiral Technologies, West Chester, PA)
CO₂ Co-solvent (Solvent B): Isopropanol with 0.25% Isopropylamine
Isocratic Method: 15% Co-solvent at 90 g/min
System Pressure: 100 bar
Column Temperature: 25 degrees C.
Sample Diluent: 3:2 Isopropanol/Methanol

### Analytical SFC Method

Column: 4.6×250 mm 3 μm Chiralpak AD-H from Chiral Technologies (West Chester, PA)
CO₂ Co-solvent (Solvent B): Isopropanol with 0.1% Isopropylamine
Isocratic Method: 10% Co-solvent at 3 mL/min System Pressure: 125 bar
Column Temperature: 40 degrees C.
Sample Diluent: Isopropanol

Because the close retention times of the enantiomers led to overlapping peaks, complete enantiomeric separation did not occur. Collection of three isolates allowed isolation of two enriched samples and a “valley.” The collected fractions were dried in a rotary evaporator at 40° C., rinsed with acetonitrile, and transferred to their final containers using methanol. Isolate one had an enantiomeric excess of 30%, chemical purity of 99.1%, and a dried weight of 227 mg as the freebase. Isolate two had an enantiomeric excess of 33.2%, chemical purity of 98.5%, and a dried weight of 250 mg as the freebase.

### Separation of R-5-MAPB and S-5-MAPB

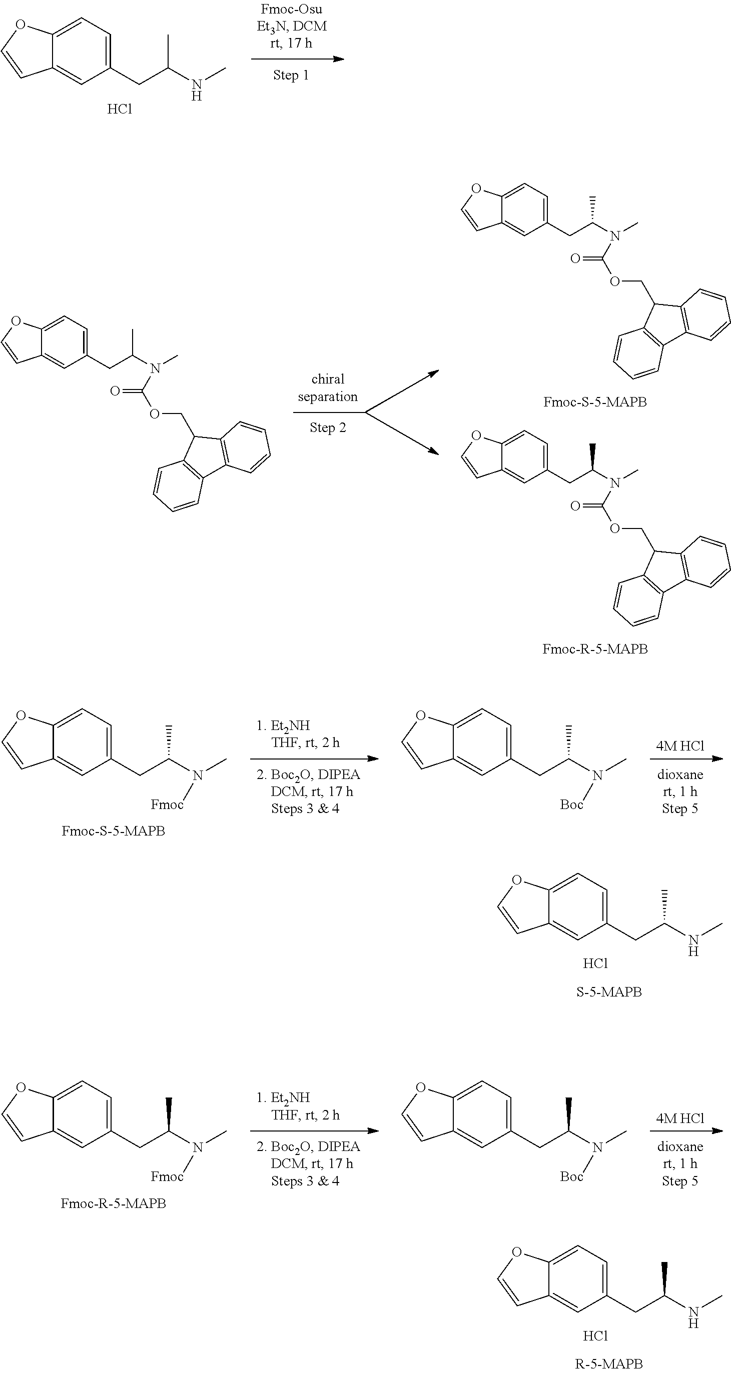

The chiral separation of Step 2 was accomplished with the following method:

### Separation SFC Method

Column: 30.0×250 mm Regis Reflect C-Amylose A, 5 (Regis Technologies, Morton Grove, IL)
Mobile Phase: 30% CO₂+70% MeOH
Flow: 30 g/min
System Pressure: 140 bar
Column Temperature: 35 degrees C.
UV: 240 nm
Diluent: Methanol

Identity of the enantiomers was confirmed with 1H NMR and LC/MS. Chromatography was used to estimate purity. The S-5-MAPB had a chemical purity of 98.49% and an enantiomeric excess of 99.46. The R-5-MAPB had a chemical purity of 88.13% and an enantiomeric excess of 99.46.

R-6-MAPB and S-6-MAPB were prepared using 6-bromobenzofuran as a starting material, as shown in the following scheme:

### Synthesis and Separation of R-6-MAPB and S-6-MAPB

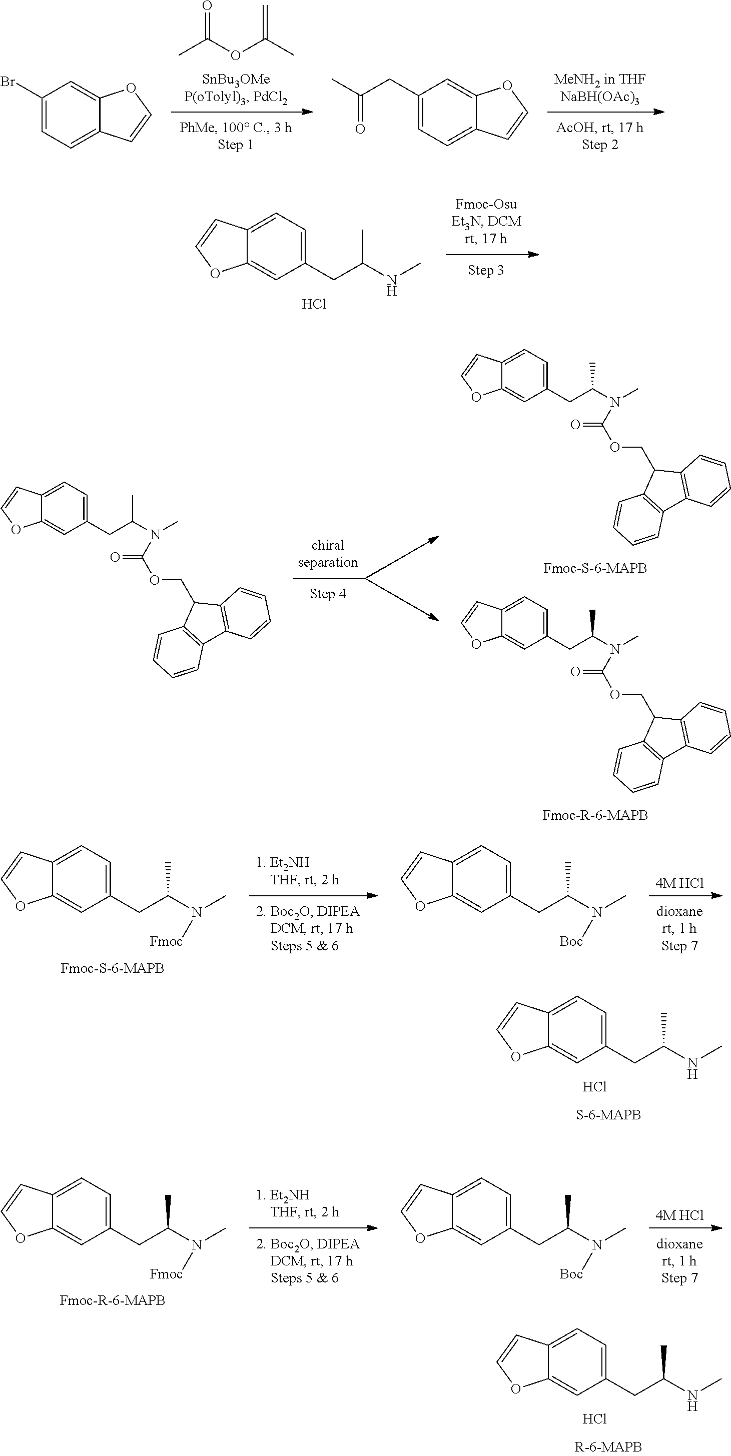

The chiral separation method used in Step 4 was the same as for S-5-MAPB and R-5-MAPB. This resulted in a sample of S-6-MAPB that had chemical purity of 98.86% and an enantiomeric excess of 100, and a sample of R-6-MAPB that had a chemical purity of 96.34% and an enantiomeric excess of 100.

### Separation of S-5-MAPB and R-5-MAPB

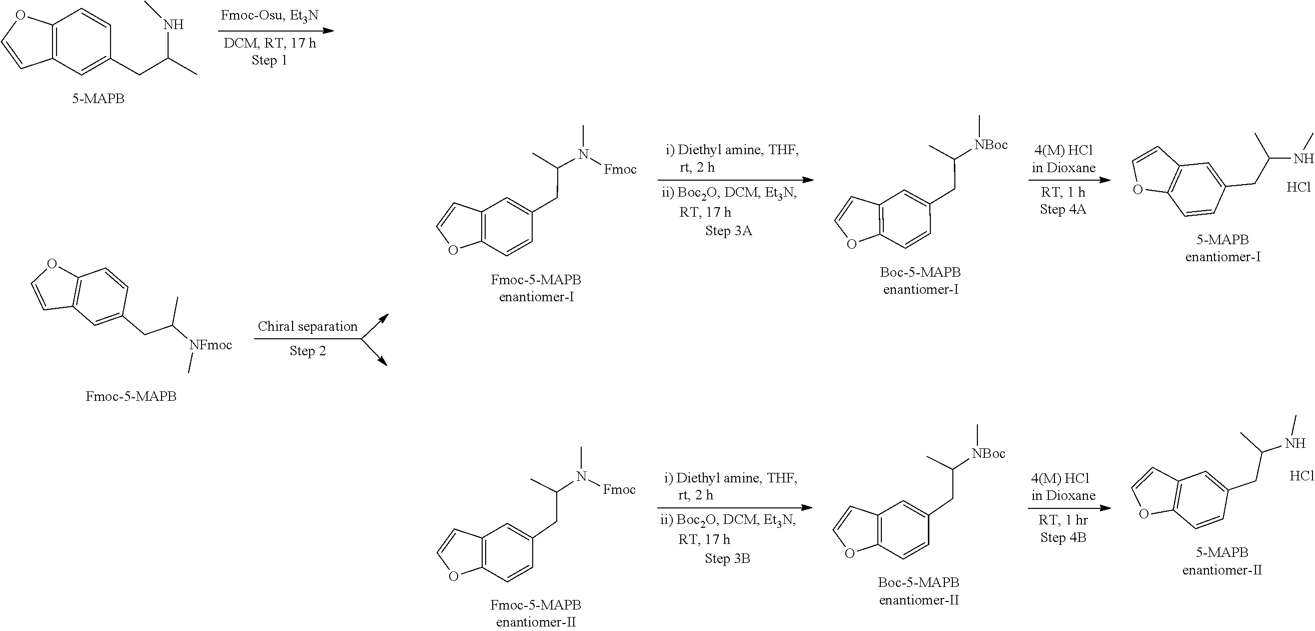

Step 1: To a stirred solution of crude 1-(benzofuran-5-yl)-N-methylpropan-2-amine (5-MAPB) (2.0 g, 10.56 mmol, 1.0 eq.) in DCM (20.0 mL) was added Et₃N (2.94 m 1, 21.13 mmol, 2.0 eq.) and Fmoc-osu (5.34 g, 15.85 mmol, 1.5 eq.) at RT and continue to stir at same temperature for 1 h. After completion of reaction (monitoring by LCMS), water (20 mL) was added to the reaction mixture, organic part was extracted with DCM (20 ml), dried over sodium sulphate, evaporated under reduced pressure to get crude, which was purified by column-chromatography using (0-10%) EA/HEX to get (9H-fluoren-9-yl)methyl (1-(benzofuran-5-yl)propan-2-yl)(methyl)carbamate (Fmoc-5-MAPB) (3.6 g, 83%) as sticky liquid.

Step 2: After chiral (SFC) separation got Fmoc-5-MAPB-enantiomer-I (1.5 g) and Fmoc-5-MAPB-enantiomer-II (1.7 g) as sticky liquid.

Fmoc-5-MAPB-enantiomer-I 1H NMR(400 MHz, DMSO-d₆) δ 7.92-7.88 (m, 2H), 7.60 (s, 1H), 7.50 (s, 1H), 7.42-7.38 (m, 4H), 7.27-7.22 (m, 2H), 7.14-7.12 (m, 1H), 6.85 (s, 2H), 4.40 (s, 1H), 4.26 (s, 1H), 4.15 (m, 1H), 3.91 (s, 1H), 2.79 (s, 1H), 2.64 (d, J=19.96 Hz, 3H), 1.23-0.76 (m, 3H), LCMS: (ES) C27H25NO3 requires 411, found 412.4 [M+H]⁺.

Fmoc-5-MAPB-enantiomer-II 1H NMR (400 MHz, DMSO-d₆) δ 7.92-7.83 (m, 2H), 7.60-7.25 (m, 8H), 7.12 (m, 1H), 6.88-6.79 (m, 2H), 4.40 (s, 1H), 4.31 (m, 1H), 4.15 (s, 1H), 3.91 (s, 1H), 2.79 (m, 1H), 2.64 (d, J=19.36 Hz, 3H) 1.28-1.06 (m, 3H). LCMS: (ES) C₂₇H₂₅NO₃ requires 411, found 412.50 [M+H]⁺.

Step 3A: To stirred solution of (9H-fluoren-9-yl)methyl (1-(benzofuran-5-yl)propan-2-yl)(methyl)carbamate (600 mg, 1.46 mmol, 1.0 eq.) in THE (20 mL) was added diethyl amine (1.52 mL, 14.59 mmol, 10.0 eq.) at RT and reaction was stir at room temperature for 16 h. After completion of reaction, solvent was evaporated, residue was re-dissolved in DCM (20 mL) and Boc-anhydride (0.67 mL, 2.92 mmol, 2.0 eq.) and Et₃N (0.82 mL, 5.839 mmol, 4.0 eq.) was added to it and stirred at room temperature for 12 h. After completion, organic part was washed with water (20 mL), dried over anhydrous sodium sulfate, evaporated under reduced pressure to get the crude which was purified with silica gel (100-200 mesh) eluted with 0-5% ethyl acetate in hexane to afford tert-butyl (1-(benzofuran-5-yl)propan-2-yl)(methyl)carbamate (7-enantiomer-I) (400 mg, 94%) as sticky colorless liquid. ¹HNMR(400 MHz, DMSO-d₆) δ 7.92 (s, 1H), 7.48 (d, J=8.32 Hz, 1H), 7.40 (s, 1H), 7.11 (d, J=8.16 Hz, 1H), 6.88 (s, 1H), 4.36-4.30 (m, 1H), 2.77 (d, J=5.6 Hz, 2H), 2.66 (s, 3H), 1.25 (s, 3H), 1.11 (s, 9H), LCMS: (ES) C₁₇H₂₃NO₃ requires 289, found 234 [M−tertbutyl]⁺.

Step 3B: To stirred solution of (9H-fluoren-9-yl)methyl (1-(benzofuran-5-yl)propan-2-yl)(methyl)carbamate (1 g, 2.43 mmol, 1.0 eq.) in THE (20 mL) was added diethyl amine (2.5 mL, 24.30 mmol, 10.0 eq.) at RT and the resulting reaction mixture was stirred at room temperature for 16 h. After completion, solvent was evaporated, residue was re-dissolved in DCM (20 mL) and Boc-anhydride (1.1 mL, 4.86 mmol, 2.0 eq.) and Et₃N (1.4 mL, 9.72 mmol, 4.0 eq.) was added to it and continue to stir at RT for 12 h. After completion, organic part was washed with water (30 mL), dried over anhydrous sodium sulfate, evaporated under reduced pressure to get the crude which was purified b column chromatography eluted with 0-5% ethyl acetate in hexane to afford pure tert-butyl (1-(benzofuran-5-yl)propan-2-yl)(methyl)carbamate (600 mg, 79%) as sticky colorless liquid. ¹HNMR(400 MHz, DMSO-d₆) δ 7.92 (d, J=1.68 HZ, 1H), 7.48 (d, J=8.04 Hz, 1H), 7.40 (s, 1H), 7.11 (d, J=8.28 Hz, 1H), 6.88 (s, 1H), 4.38-4.30 (m, 1H), 2.77 (d, J=5.8 Hz, 2H), 2.64 (s, 3H), 1.25 (s, 3H), 1.11 (s, 9H), LCMS: (ES) C₁₇H₂₃NO₃ requires 289, found 234 [M−tertbutyl]⁺.

Step 4A: To a stirred solution tert-butyl (1-(benzofuran-5-yl)propan-2-yl)(methyl)carbamate (7-enantiomer-1) (1.8 g, 6.228 mmol, 1 eq.) in 1,4 dioxane (10 ml) was added 4(M) HCl in 1,4 dioxane (15 mL) at 0° C. and the resulting reaction mixture was allowed to stir at room temperature for 1h. Upon completion of reaction (monitored by TLC, 30% EA in Hexane), the solvent were evaporated and the crude was washed twice with diethyl ether (2×30 mL) and pentane finally dried under vacuum to afford 1-(benzofuran-5-yl)-N-methylpropan-2-amine hydrochloride (1.1 g, 93%) as white solid. ¹HNMR(400 MHz, DMSO-d₆) δ 8.87-8.82 (bs, 2H), 7.99 (s, 1H), 7.57 (m, 2H), 7.21 (d, J=8.28 Hz, 1H), 6.93 (S, 1H), 3.39 (bs, 1H), 3.26 (q, 1H), 2.77 (q, 3H), 2.57 (s, 3H), 1.11 (d, J=6.4 Hz, 3H). LCMS: (ES) C₁₂H₁₅NO requires 189, found 190 [M+H]⁺. HPLC: Purity (λ250 nm): 99.64%. Absolute configuration determined by comparison to authentic samples.

Step 4B: To a stirred solution of tert-butyl (1-(benzofuran-5-yl)propan-2-yl)(methyl)carbamate (7-enantiomer-II) (1.7 g, 5.87 mmol, 1 eq.) in 1,4 dioxane (15 mL) was added 4(M) HCl in 1,4 dioxane (10 mL) at 0° C. and the resulting reaction mixture was allowed to stir at room temperature for 1h. Upon completion of reaction (monitored by TLC, 30% EA in Hexane), the solvent were evaporated and the crude was washed twice with diethyl ether (2×30 mL) and pentane and dried under vacuum to afford (R)-1-(benzofuran-5-yl)-N-methylpropan-2-amine hydrochloride (Compound-9-enantiomer-II) (1 g, 99%) as white solid. ¹HNMR(400 MHz, DMSO-d₆) δ 8.82 (bs, 2H), 7.99 (d, J=2.12 HZ, 1H), 7.55 (t, J=8.44 HZ, 6.56 Hz, 2H), 7.21 (dd, J=1.08 Hz, 8.32 Hz, 1H), 6.93 (d, J=1.44 HZ, 1H), 3.39 (bs, 1H), 3.25-3.21 (q, 1H), 2.77-2.71 (q, 1H), 2.57 (s, 3H), 1.10 (t, J=6.48 Hz, 12.12 Hz, 3H). LCMS: (ES) C₁₂H₁₅NO requires 189, found 190.1 [M+H]⁺. HPLC: Purity (λ210 nm): 99.84%. Absolute configuration determined by comparison to authentic samples.

### Separation of S-6-MAPB and R-6-MAPB

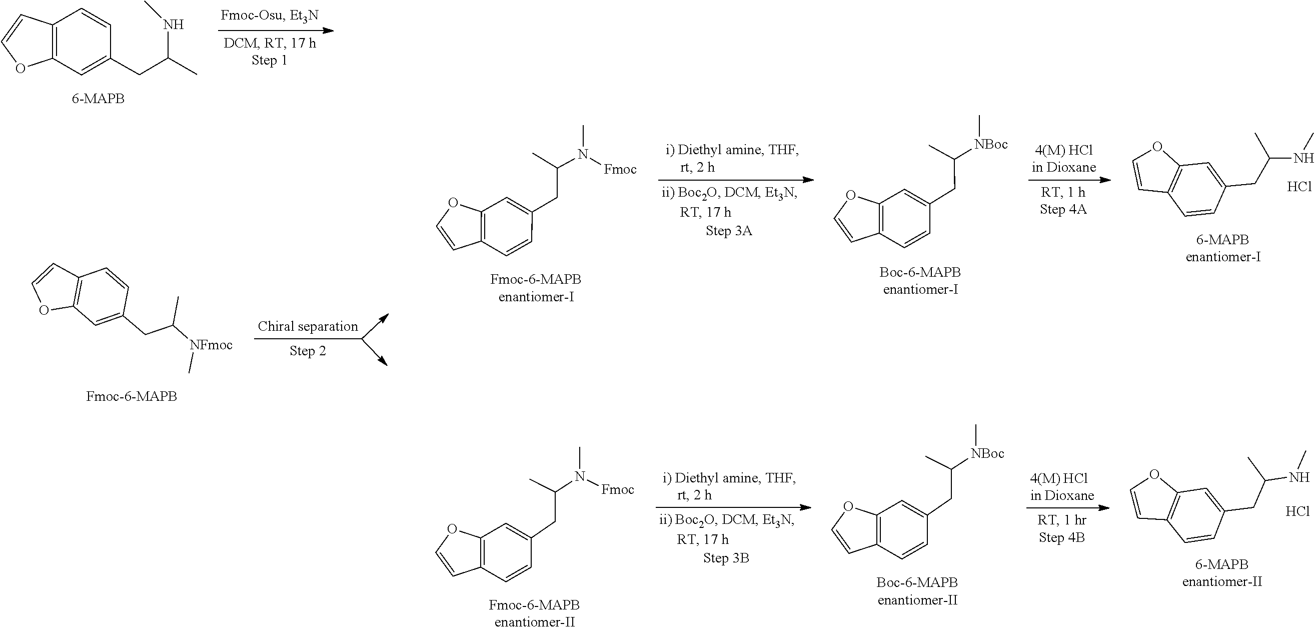

Step 1: To a stirred solution of crude 1-(benzofuran-6-yl)-N-methylpropan-2-amine (6-MAPB) (2.5 g, 13.22 mmol) in DCM (30 mL) was added Et₃N (9.27 mL, 66.13 mmol) and Fmoc-Osu (5.34 gm, 15.87 mmol). Resultant reaction mixture was stirred at room temperature for 17h. After completion, reaction mixture was washed water (10 mL) and organic layer was concentrated under reduced pressure to get the crude which was purified by silica gel (100-200 mesh) column chromatography eluted with 10-20% ethyl acetate in hexane to get (9H-fluoren-9-yl)methyl (1-(benzofuran-6-yl)propan-2-yl)(methyl)carbamate (4.5 g, 82.6%) as a colorless sticky liquid.

Step 2: Isomer separation of Int-3 was done by SFC.

The method of SFC separation was given below
Column: REGIS REFLECT C-Amylose A (30.0×250 mm), 5p
Flow: 30 g/min
Mobile Phase:30% CO₂+70% MeOH
ABPR: 140 bar
Temp:35° C.
UV:240 nm
DILUENT: MeOH

6.0 g crude was separated by SFC and ˜2.5 g of each fraction (Peak-1 and Peak-2) was obtained.

Peak 1 was obtained at 4.83 min and Peak 2 was obtained at 5.63 min. We observed Fmoc group was removed during chiral separation and generated impurities along with desired compound.

### Peak-1 (6-MAPB Enantiomer-I)

¹H NMR (DMSO-d₆): δ 7.91 (m, 2H), 7.60-7.12 (m, 6H), 6.85 (bs, 1H), 4.40-4.16 (m, 1H), 4.31 (s, 3H), 2.81 (d, J=7.0 Hz, 1H), 2.64 (d, J=17.92 Hz, 1H), 1.14-0.81 (m, 3H). LCMS: (ES) C₂₇H₂₅NO₃ requires 411.18, found 412.3 [M+H]⁺.

### Peak-2 (6-MAPB Enantiomer-II)

¹H NMR (DMSO-d₆): δ 7.92-7.83 (m, 2H), 7.58-7.32 (m, 4H), 7.27 (bs, 1H), 7.12 (m, 1H), 6.85 (s, 1H), 4.40-4.16 (m, 3H), 2.81 (d, J=7.0 Hz, 1H), 2.64 (d, J=17.18 Hz, 2H), 1.08-0.81 (m, 3H). LCMS: (ES) C₂₇H₂₅NO₃ requires 411, found 412 [M+H]⁺.

Step 3A and 3B: Each Fmoc protected enantiomer of 6-MAPB (2.5 g, 6 mmol, not fully pure) in THE (20 mL) was treated with diethyl amine (4 0.4 mL, 60 mmol) and stirred at room temperature for 4 h. After completion, [Monitored with TLC, Mobile Phase 10% EtOAc-hexane], solvent was evaporated, residue was re-dissolved in DCM (30 mL) and then Boc-anhydride (2.7 mL, 11.84 mmol) and Et₃N (3.3 mL, 23.68 mmol) was added to it and stirred at room temperature for 17 h. After completion, organic part was washed with water (10 mL), dried over anhydrous sodium sulfate, evaporated under reduced pressure to get the crude which was purified with silica gel (100-200 mesh) elute with 0-5% ethyl acetate hexane to afford boc-6-MAPB enantiomer I and II (1.5 g, 85%) as a sticky colorless liquid.

boc-6-MAPB enantiomer I ¹H NMR (400 MHz, DMSO-d₆): δ 7.90 (d, J=1.68 Hz, 1H), 7.53 (d, J=7.92 Hz, 1H), 7.36 (s, 1H), 7.07 (d, J=7.84 Hz, 1H), 6.88 (s, 1H), 4.33 (s, 1H), 2.79 (s, 2H), 2.63 (s, 3H), 1.24 (s, 3H), 1.10 (s, 9H). LCMS: (ES) C17H23NO3 requires 289.17, found 290.3 [M+H]+.

boc-6-MAPB enantiomer II ¹H NMR (400 MHz, DMSO-d₆): δ 7.90 (d, J=1.8 Hz, 1H), 7.53 (d, J=7.8 Hz, 1H), 7.36 (s, 1H), 7.07 (d, J=7.84 Hz, 1H), 6.88 (s, 1H), 4.33 (bs, 1H), 2.79 (s, 2H), 2.63 (s, 3H), 1.26 (d, J=6.32 Hz, 3H), 1.10 (s, 9H). LCMS: (ES) C17H23NO3 requires 289.17, found 290.1 [M+H]+.

Step 4A and 4B: (2.0 g, 6.92 mmol, 1 eq.) were separately dissolved in 1, 4 dioxane (5 mL) and 4M HCl in 1, 4 dioxane (20 mL) was added to it. Resultant solution was stirred at room temperature for 3 h. After completion, solvent was evaporated; residue was triturated with hexane to get the pure desired amine HCl salt as a white solid 6-MAPB enantiomer I (1.24 g, 94%) and 6-MAPB enantiomer II (1.25 g, 95.44%).

6-MAPB enantiomer I ¹H NMR (400 MHz, DMSO-d₆): δ 9.05 (s, 2H), 7.96 (d, J=1.96 Hz, 1H), 7.62 (d, J=7.92 Hz, 1H), 7.53 (s, 1H), 7.16 (d, J=7.84 Hz, 1H), 6.93 (s, 1H), 3.41-3.37 (m, 1H), 3.30-3.26 (m, 1H), 2.80-2.75 (q, 1H), 2.56 (t, J=5.16 Hz, 5.24 Hz, 3H), 1.12 (d, J=6.44 Hz, 3H). LCMS: (ES) C12H15NO requires 189.12, found 190.38 [M+H]+. HPLC: Purity (λ210 nm): 98.86%. Chiral HPLC: Purity (λ 250 nm): 100%. Absolute stereochemistry assigned by comparison to authentic sample.

6-MAPB enantiomer II ¹H NMR (400 MHz, DMSO-d₆): δ 9.01 (bs, 2H), 7.96 (d, J=2.2 Hz, 1H), 7.62 (d, J=7.92 Hz, 1H), 7.53 (s, 1H), 7.16 (d, J=7.96 Hz, 1H), 6.93 (t, J=1.48 Hz, J=0.6 Hz, 1H), 3.30-3.25 (m, 1H), 2.80-2.75 (q, 1H), 2.57-2.55 (m, 3H), 1.12 (d, J=6.48 Hz, 3H). LCMS: (ES) C12H15NO requires 189.12, found 190.29 [M+H]⁺. HPLC:Purity (λ 260 nm): 96.34%. Chiral HPLC: Purity (λ 250 nm): 99.93%. Absolute stereochemistry assigned by comparison to authentic sample.

### Separation of S-5-MBPB and R-5-MBPB

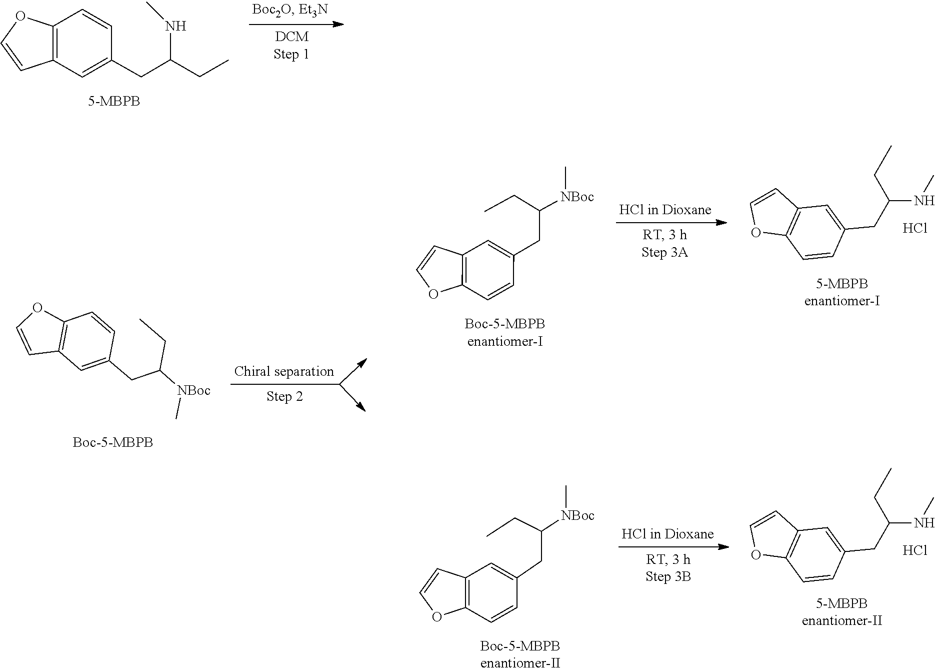

Step 1: To a stirred solution of 1-(benzofuran-5-yl)-N-methylbutan-2-amine (5-MBPB) (3.3 g, 17.55 mmol, 1.0 eq) in DCM (20 mL) was added TEA (7.38 mL, 52.66 mmol, 3.0 eq). Then Boc anhydride (6.04 mL, 26.33 mmol, 1.5 eq) was added to the reaction mixture at 0° C. and stirred at RT for overnight. After the completion [Monitored with TLC, Mobile Phase 5% EtOAc-hexane, Rf-0.5], reaction mixture was diluted with DCM (100 mL) and washed with water (20 mL), and finally NaCl solution. DCM part was dried over magnesium sulphate and concentrated under reduced pressure to afford tert-butyl (1-(benzofuran-5-yl)butan-2-yl)(methyl)carbamate (Boc-5-MBPB) (4.8 g, 90%) as a light yellow liquid. ¹H NMR (400 MHz, DMSO-d₆): δ 7.95 (d, J=15.16 Hz, 1H), 7.47-7.40 (m, 2H), 7.10 (d, J=8.2 Hz, 1H), 6.87 (s, 1H), 4.20-4.09 (m, 1H), 2.79-2.69 (m, 2H), 2.59 (s, 3H), 1.51-1.46 (m, 2H), 1.23 (s, 3H), 1.10 (s, 6H), 0.91-0.77 (m, 3H).

Step 2: Isomer separation of Boc-5-MBPB was done by SFC.
The method of SFC separation was given below

### Method of SFC:

Column Name. Chiralpak AY-H (250×21 mm) 5p
Flow rate 21.0 ml/min
Mobile phase Hexane/EtOH/IPAmine-80/20/0.1
Solubility MeOH
Wave length 246 nm
Run time 25 min

4.8 g crude was submitted and after separation ˜1.8 g of enantiomer I and enantiomer II was obtained.
Enantiomer I was obtained at −4.13 min
Enantiomer II was obtained at −5.57 min

Boc-5-MBPB enantiomer I ¹H NMR (400 MHz, DMSO-d₆): δ 7.91 (s, 1H), 7.47-7.40 (m, 2H), 7.10 (d, J=8.68 Hz, 1H), 6.87 (s, 1H), 4.21-4.09 (m, 1H), 2.79-2.69 (m, 2H), 2.59 (s, 3H), 1.51 (m, 2H), 1.23-1.10 (m, 9H), 0.85-0.79 (m, 3H). Rotamers observed. LCMS: (ES) C18H25NO3 requires 303, found 204 [M-Boc+H]⁺.

Boc-5-MBPB enantiomer II ¹H NMR (400 MHz, DMSO-d₆): δ 7.91 (s, 1H), 7.48-7.40 (m, 2H), 7.10 (d, J=8.12 Hz, 1H), 6.87 (s, 1H), 4.29-4.08 (m, 1H), 2.79-2.69 (m, 2H), 2.59 (s, 3H), 1.51 (m, 2H), 1.23-1.10 (m, 9H), 0.85-0.79 (m, 3H). LCMS: (ES) C18H25NO3 requires 303, found 204 [M-Boc+H]⁺.

Step 3A and 3B: After chiral separation Boc-5-MBPB enantiomer I and Boc-5-MBPB enantiomer II (1.7 g, 5.6 mmol, 1.0 eq.) were separately dissolved in 1, 4 dioxane (5 mL) and 4M HCl in 1, 4 dioxane (20 mL) was added to it. Resultant solution was stirred at room temperature for 3 h. After completion, solvent was evaporated; residue was triturated with hexane to get the pure desired amine HCl salt as a white solid 5-MBPB enantiomer I (1.3 g, ˜100%) and 5-MBPB enantiomer I (1.1 g, 96%).

5-MBPB enantiomer I ¹H NMR (400 MHz, DMSO-d₆): δ 8.97 (s, 1H), 8.82 (s, 1H), 7.99 (d, J=2 Hz, 1H), 7.57 (d, J=8.8 Hz, 2H), 7.24 (d, J=8.36 Hz, 1H), 6.93 (d, J=1.24 Hz, 1H), 3.33 (s, 1H), 3.19-3.14 (m, 1H), 2.91-2.85 (m, 1H), 2.55 (s, 3H), 1.59-1.51 (m, 2H), 0.89 (t, J=7.44 Hz, J=7.48 Hz, 3H). LCMS: (ES) C13H17NO requires 203, found 204 [M+H]⁺. HPLC: Purity (λ 240 nm): 99.65%.

5-MBPB enantiomer II ¹H NMR (400 MHz, DMSO-d₆): δ 8.93 (s, 1H), 8.79 (s, 1H), 7.99 (d, J=2 Hz, 1H), 7.57 (d, J=8.76 Hz, 2H), 7.24 (d, J=8.36 Hz, 1H), 6.93 (d, J=1.4 Hz, 1H), 3.33 (s, 1H), 3.19-3.14 (m, 1H), 2.91-2.85 (m, 1H), 2.55 (s, 3H), 1.59-1.49 (m, 2H), 0.89 (t, J=7.44 Hz, J=7.48 Hz, 3H). LCMS: (ES) C13H17NO requires 203, found 204.1 [M+H]⁺. HPLC: Purity (λ 250 nm): 99.81%.

### Separation of S-6-MBPB and R-6-MBPB

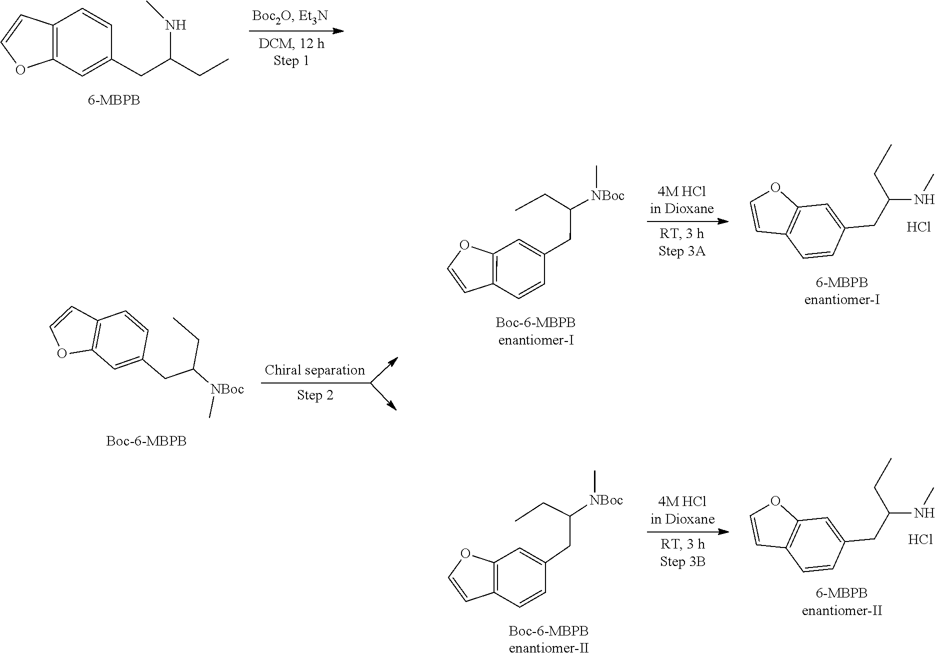

Step 1: To a stirred solution of 1-(benzofuran-6-yl)-N-methylbutan-2-amine (6-MBPB) (5.0 g, 19.70 mmol, 1.0 eq.) in DCM (15 mL) was added TEA (8.29 mL, 59.11 mmol, 3.0 eq.). Then Boc anhydride (6.78 mL, 29.55 mmol, 1.5 eq.) was added to the reaction mixture at 0° C. and stirred at RT for 12 h. After completion [monitored by TLC, mobile Phase 5% EtOAc-hexane]reaction mixture was diluted with DCM (100 mL) and washed with water (20 mL), followed by NaCl solution. Organic layer was dried over magnesium sulphate and concentrated under reduced pressure to afford tert-butyl (1-(benzofuran-6-yl)butan-2-yl)(methyl)carbamate (Boc-6-MPBP) (6.7 g, 83%) as a light yellow liquid. ¹H NMR (400 MHz, DMSO-d₆): δ 7.89 (s, 1H), 7.51 (t, J=7.72 Hz, J=7.32 Hz, 1H), 7.35 (s, 1H), 7.06 (d, J=7.96 Hz, 1H), 6.88 (s, 1H), 4.22-4.12 (m, 1H), 2.86-2.71 (m, 2H), 2.59 (s, 3H), 1.54-1.48 (m, 2H), 1.26-1.10 (m, 9H), 0.82-0.76 (m, 3H). Rotamers observed. LCMS: (ES) C18H25NO3 requires 303, found 304.14 [M+H]⁺. HPLC: Purity (λ 210 nm): 99.70%. Chiral HPLC: Purity (λ 250 nm): 52.87% and Purity (λ 250 nm): 47.13%.

Step 2: Isomer separation of Int-9 was done by SFC.

The method of SFC separation was given below
Column Name:Chiralpak AY-H (250×21 mm) 5p
Flow rate:21.0 ml/min
Mobile phase:Hexane/EtOH/IPAmine-80/20/0.1
Solubility:MeOH
Wave length:246 nm
Run time:25 min

5.0 g crude was separated by SFC and ˜2.2 g of each fraction (enantiomer I and enantiomer II) was obtained.

Enantiomer I was obtained at −3.78 min and enantiomer II was obtained at −9.29 min.

Boc-6-MPBP enantiomer I ¹H NMR (400 MHz, DMSO-d₆): δ 7.89 (s, 1H), 7.51 (t, J=7.72 Hz, J=7.32 Hz, 1H), 7.36 (s, 1H), 7.06 (d, J=7.96 Hz, 1H), 6.88 (bs, 1H), 4.22-4.12 (m, 1H), 3.01-2.71 (m, 2H), 2.59 (s, 3H), 1.54-1.42 (m, 2H), 1.26-1.10 (m, 9H), 0.82 (d, J=7.32 Hz, 3H). Rotamers observed. LCMS: (ES) C18H25NO3 requires 303, found 204.12 [M −Boc H]⁺. HPLC: Purity (λ 220 nm): 95.08%. Chiral HPLC: Purity (λ 250 nm): 100%.

Boc-6-MPBP enantiomer II ¹H NMR (400 MHz, DMSO-d₆): δ 7.89 (s, 1H), 7.51 (t, J=7.72 Hz, J=7.32 Hz, 1H), 7.36 (s, 1H), 7.06 (d, J=7.96 Hz, 1H), 6.88 (s, 1H), 4.22-4.12 (m, 1H), 2.84-2.71 (m, 2H), 2.59 (s, 3H), 1.54-1.48 (m, 2H), 1.22-1.10 (m, 9H), 0.82-0.75 (m, 3H). Rotamers observed. LCMS: (ES) C18H25NO3 requires 303, found 204.12 [M −Boc H]⁺. HPLC: Purity (λ 210 nm): 99.68%. Chiral HPLC: Purity (λ 250 nm): 99.95%.

Step 3A and 3B: After chiral separation Boc-6-MPBP enantiomer I and Boc-6-MPBP enantiomer II (2.2 g, 7.26 mmol, 1 eq.) were separately dissolved in 1, 4 dioxane (5 mL) and to it was added 4M HCl in 1, 4 dioxane (20 mL). Resultant solution was stirred at room temperature for 3 h. After completion, solvent was evaporated; residue was triturated with hexane to get the pure desired amine HCl salt as a white solid 6-MPBP enantiomer I (2.05 g, 95.49%) and 6-MPBP enantiomer II (2.02 g, 94.09%).

6-MPBP enantiomer I ¹H NMR (400 MHz, DMSO-d₆): δ 9.08-8.91 (m, 2H), 7.96 (d, J=2.08 Hz, 1H), 7.62 (d, J=7.92 Hz, 1H), 7.57 (s, 1H), 7.20 (d, J=7.88 Hz, 1H), 6.93 (d, J=1.8 Hz, 1H), 3.23-3.18 (m, 1H), 2.94-2.88 (m, 1H), 2.55-2.53 (m, 3H), 1.62-1.52 (m, 2H), 0.903 (t, J=7.44 Hz, 7.56 Hz, 3H). LCMS: (ES) C13H17NO requires 203, found 204 [M+H]⁺. HPLC: Purity (λ 210 nm): 97.42%. Chiral HPLC: Purity (λ 250 nm): 100%.

6-MPBP enantiomer II ¹H NMR (400 MHz, DMSO-d₆): δ 8.98-8.83 (m, 2H), 7.96 (d, J=2.16 Hz, 1H), 7.62 (d, J=7.92 Hz, 1H), 7.57 (s, 1H), 7.19 (d, J=7.84 Hz, 1H), 6.93 (d, J=1.84 Hz, 1H), 3.56 (s, 1H), 3.38-3.16 (m, 1H), 2.94-2.88 (m, 1H), 2.55 (t, J=3.2 Hz, J=5.28 Hz, 3H), 1.62-1.50 (m, 2H), 0.92 (t, J=7.44 Hz, 3H). LCMS: (ES) C13H17NO requires 203, found 204 [M+H]⁺. HPLC: Purity (λ 240 nm): 99.59%. Chiral HPLC: Purity (λ 250 nm): 100%.

### Determination of Specific Rotation

Specific rotation was determined for individual enantiomers using a Jasco P-2000 Polarimeter, 589 nm Na lamp (Path Length 1 dm, 20° C. temperature, Concentration approx. 1 g/100 mL). EtOH was used as solvent for beta-ketone compounds, while distilled water was used for other compounds. Ten measurements were made for each compound. 6-MBPB enantiomer 1 was found to have a specific rotation of 19.6 with a standard deviation of 0.9.

### Separation of Bk-5-MAPB

Bk-5-MAPB was Boc-protected. Next, isomeric separation of Boc-Bk-5-MAPB was conducted using the SFC and after chiral separation, both isomers of Boc-Bk-5-MAPB were deprotected to afford (−)-Bk-5-MAPB and (+)-Bk-5-MAPB. Each procedure is described below.

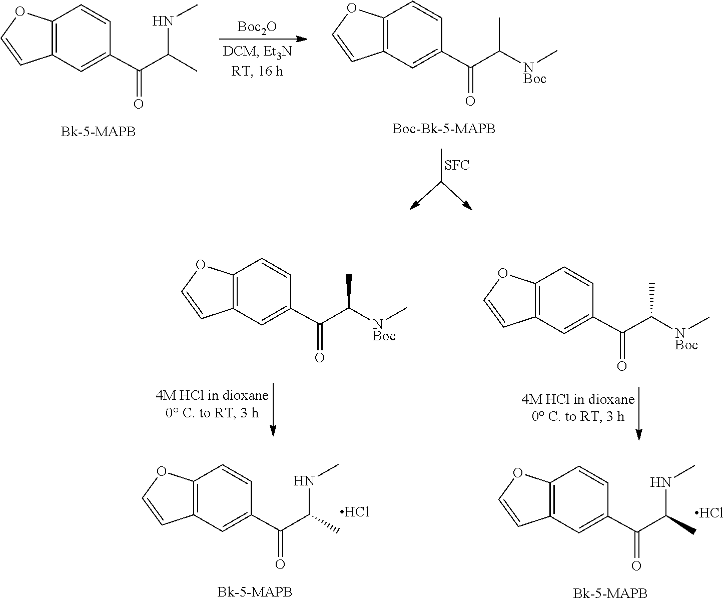

Synthesis of Boc-Bk-5-MAPB: To a stirred solution of 1-(benzofuran-5-yl)-2-(methylamino) propan-1-one (16-5) (5.2 g, 25.61 mmol, 1eq.) in dry DCM (50 ml) was added triethylamine (7.39 ml, 51.23 mmol, 2eq.) and Boc anhydride (11.75 ml, 51.23 mmol, 2 eq.) and the resulting reaction mixture was allowed to stir at room temperature for 4 hours. Upon completion of reaction (monitored by TLC, 10% EA in hexane), the reaction mixture was extracted with DCM (2×100 ml) and washed with water followed by brine solution. Combined organic solvent was dried over anhydrous sodium sulphate and solvent was evaporated under vacuum and purified by silica gel column chromatography using ethyl acetate/hexane (10:90 v/v) as eluent to afford pure tert-butyl (1-(benzofuran-5-yl)-1-oxopropan-2-yl)(methyl)carbamate (Boc-Bk-5-MAPB) as yellow sticky gum (3.9 g, 50%). ¹H NMR (400 MHz, CDCl₃) δ 8.33 (s, 1H), 7.99 (d, J=8.52 Hz, 1H), 7.66 (bs, 1H), 7.52 (d, J=8.56 Hz, 1H), 6.81 (d, J=1.12 Hz, 1H), 5.80 (q, 1H), 2.59 (s, 3H), 1.43 (s, 9H), 1.37 (m, 3H). LCMS: (ES) C₁₇H₂₁NO₄ requires 303, found 304 [M+H]⁺.

### Isomeric Separation by SFC:

Isomeric separation of intermediate Boc-Bk-5-MAPB was performed using SFC and the method of SFC separation is given below:
Column: (R,R) Whelk-01 (4.5 mm×250 mm), 5μ
Flow: 2 g/min
Mobile Phase: 75% CO2+25% (ISOPROPANOL)
ABPR: 100 bar
Temp: 35° C.
UV: 220 nm
Diluent: IPA

After SFC separation, 1.8 g of intermediate Boc-Bk-5-MAPB-Isomer-1 and 1.9 g of intermediate Boc-Bk-5-MAPB-Isomer-2 were isolated. Characterization of intermediate Boc-Bk-5-MAPB-Isomer-1 and intermediate Boc-Bk-5-MAPB-Isomer-2 are below:

Boc-Bk-5-MAPB-Isomer-1: ¹HNMR (400 MHz, CDCl₃) δ 8.33-8.22 (bs, 1H), 8.00-7.93 (m, 1H), 7.66 (bs, 1H), 7.52 (d, J=7.96 Hz, 1H), 6.82 (s, 1H), 5.79-5.28 (m, 1H), 2.77-2.59 (s, 3H), 1.44 (s, 9H), 1.38 (m, 3H). Rotamers observed. LCMS: (ES) C17H21NO4 requires 303, found 304 [M+H]+. Chiral-HPLC: Purity (λ 235 nm): 99.12%.

Boc-Bk-5-MAPB-Isomer-2: ¹HNMR (400 MHz, CDCl3) δ 8.30-8.22 (bs, 1H), 8.00-7.91 (m, 1H), 7.66 (bs, 1H), 7.52 (d, J=8.48 Hz, 1H), 6.82 (s, 1H), 5.79-5.28 (m, 1H), 2.77-2.60 (s, 3H), 1.44 (s, 9H), 1.38 (m, 3H). Rotamers observed, LCMS: (ES) C17H21NO4 requires 303, found 304 [M+H]+. Chiral-HPLC: Purity (λ 235 nm): 100%.

Synthesis (−)-Bk-5-MAPB and (+)-Bk-5-MAPB: Both chiral intermediates were then subsequently de-protected using 4(M) HCl in 1,4 dioxane as described in Synthesis 16 to afford the two isomers of Bk-5-MAPB. Characterization of (−)-Bk-5-MAPB-isomer-1 and (+)-Bk-5-MAPB-isomer-2 are below:

(−)-Bk-5-MAPB-Isomer-1: Following the deprotection, Bk-5-MAPB Isomer-1 was afforded as an off-white solid (1.1 g, 96%). ¹HNMR (400 MHz, DMSO) δ 9.60 (s, 1H), 9.16 (bs, 1H), 8.46 (s, 1H), 8.19 (d, J=1.8 Hz, 1H), 8.02 (d, J=7.56 Hz, 1H), 7.82 (d, J=8.6 Hz, 1H), 7.15 (d, J=1.04 Hz, 1H), 5.25 (d, J=7.04 Hz, 1H), 2.61 (s, 3H), 1.50 (d, J=7.04 Hz, 3H). LCMS: (ES) C12H13NO2 requires 203, found 203.9 [M+H]+. HPLC: Purity (λ 230 nm): 99.19%.

(+)-Bk-5-MAPB-Isomer-1: Following the deprotection, Bk-5-MAPB Isomer-2 was afforded as an off-white solid (1.1 g, 96%). ¹H NMR (400 MHz, DMSO) δ 9.59 (s, 1H), 9.14 (s, 1H), 8.46 (d, J=1.44 Hz, 1H), 8.19 (d, J=2.16 Hz, 1H), 8.02 (dd, J=1.72 Hz, 8.72 Hz, 1H), 7.82 (d, J=8.68 Hz, 1H), 7.15 (d, J=1.84 Hz, 1H), 5.27 (q, 1H), 2.61 (s, 3H), 1.50 (d, J=7.08 Hz, 3H). LCMS: (ES) C12H13NO2 requires 203, found 204 [M+H]⁺. HPLC: Purity (λ 240 nm): 99.24%.

### Separation of Bk-6-MAPB

Bk-6-MAPB was Boc-protected. Next, isomeric separation of Boc-Bk-6-MAPB was conducted using the SFC and after chiral separation, both isomers of Boc-Bk-6-MAPB were deprotected to afford (−)-Bk-6-MAPB and (+)-Bk-6-MAPB. Each procedure is described below.

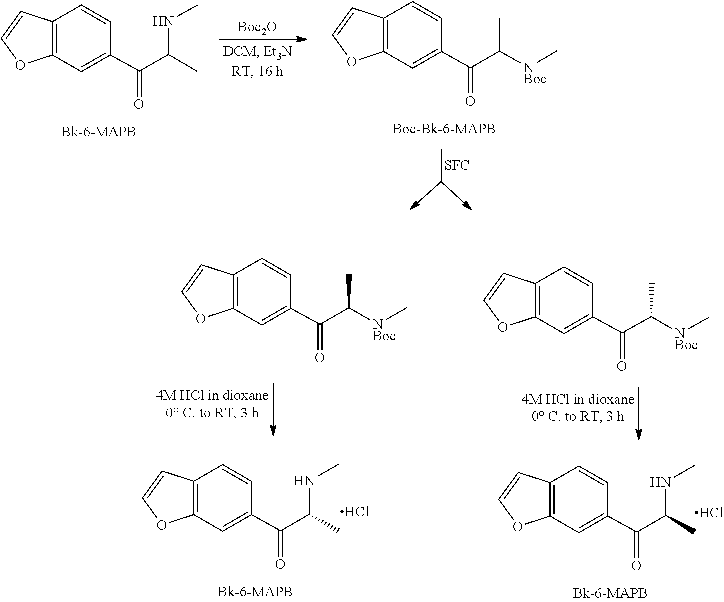

Synthesis of Boc-Bk-6-MAPB: To a stirred solution of 1-(benzofuran-6-yl)-2-(methylamino) propan-1-one (Bk-6-MAPB) (3 g, 14.77 mmol, 1eq.) in dry DCM (30 ml) was added triethylamine (4.26 ml, 29.55 mmol, 2 eq.) and Boc anhydride (6.78 ml, 29.55 mmol, 2 eq.) and the resulting reaction mixture was allowed to stir at room temperature for 4 hours. Upon completion of the reaction (monitored by TLC, 10% EA in hexane), the reaction mixture was extracted with DCM (2×50 ml) and washed with water followed by brine solution. The combined organic solvent was dried over anhydrous sodium sulphate and the solvent was evaporated under vacuum and purified by silica gel column chromatography using ethyl acetate/hexane (10:90 v/v) as eluent to afford tert-butyl (1-(benzofuran-6-yl)-1-oxopropan-2-yl)(methyl) carbamate (Boc-Bk-6-MAPB) as a yellow sticky gum (2.5 g, 55%). 1H NMR(400 MHz, CDCl₃) δ 8.20-8.11 (bs, 1H), 7.93-7.85 (bd, 1H), 7.76 (s, 1H), 7.63 (bs, 1H), 6.80 (s, 1H), 5.77-5.31 (m, 1H), 2.76-2.58 (s, 3H), 1.45 (s, 9H), 1.38 (m, 3H). Rotamers observed. LCMS: (ES) C₁₇H₂₁NO₄ requires 303, found 304 [M+H]⁺.

### Isomeric Separation by SFC:

Isomer separation of Boc-Bk-6-MAPB was performed using SFC and the method of SFC separation is given below:
Column: (R,R) Whelk-01 (4.5 mm×250 mm), 5μ
Flow: 2 g/min
Mobile Phase: 75% CO2+25% (isopropanol)
ABPR: 100 bar
Temp: 35° C.
UV: 220 nm
Diluent: IPA

After SFC separation 1.5 g of Boc-Bk-6-MAPB-Isomer-1 and 1.2 g of Boc-Bk-6-MAPB-Isomer-2 were isolated. Characterization of intermediate Boc-Bk-6-MAPB-Isomer-1 and intermediate Boc-Bk-6-MAPB-Isomer-2 are below:

Boc-Bk-6-MAPB-Isomer-1: ¹H NMR (400 MHz, CDCl₃) δ 8.20-8.11 (s, 1H), 7.93-7.84 (dd, J=8.36 Hz, 1H), 7.76 (s, 1H), 7.63 (d, J=7.8 Hz, 1H), 6.81 (s, 1H), 5.78-5.28 (m, 1H), 2.77-2.61 (s, 3H), 1.45 (s, 9H), 1.38 (m, 3H). Rotamers observed. LCMS: (ES) C17H21NO4 requires 303, found 304 [M+H]+.

Boc-Bk-6-MAPB-Isomer-2: ¹H NMR (400 MHz, CDCl₃) δ 8.20-8.11 (s, 1H), 7.93-7.83 (dd, J=8.04 Hz, 30.44 Hz, 1H), 7.76 (s, 1H), 7.63 (d, J=7.84 Hz, 1H), 6.81 (s, 1H), 5.77-5.28 (m, 1H), 2.77-2.61 (s, 3H), 1.45 (s, 9H), 1.38 (m, 3H). Rotamers observed. LCMS: MS(ES) C17H21NO4 requires 303, found 304 [M+H]⁺.

Synthesis (−)-Bk-6-MAPB and (+)-Bk-6-MAPB: Both chiral intermediates were then subsequently de-protected using 4(M) HCl in 1,4 dioxane as described in Synthesis 17 to afford the two isomers of Bk-6-MAPB. Characterization of (−)-Bk-6-MAPB-isomer-1 and (+)-Bk-6-MAPB-isomer-2 are below:

(−)-Bk-6-MAPB: Following the deprotection, (−)-Bk-6-MAPB was afforded as a white solid (1.1 g, 87%). ¹H NMR (400 MHz, CDCl₃) δ 10.65 (s, 1H), 9.26 (s, 1H), 8.13 (s, 1H), 7.85-7.82 (m, 2H), 7.71 (d, J=8.16 Hz, 1H), 6.85 (d, J=1.7 Hz, 1H), 5.01 (bs, 1H), 2.89 (bs, 3H), 1.84 (d, J=7 Hz, 3H). LCMS: (ES) C12H13NO2 requires 203, found 204 [M+H]⁺. HPLC: Purity (λ 300 nm): 99.63%.

(+)-Bk-6-MAPB: Following the deprotection, (+)-Bk-6-MAPB was afforded as a white solid (1.1 g, 92%). ¹H NMR (400 MHz, CDCl₃) δ 10.57 (s, 1H), 9.33 (s, 1H), 8.13 (s, 1H), 7.85-7.82 (m, 2H), 7.70 (d, J=8.08 Hz, 1H), 6.85 (d, J=1.9 Hz, 1H), 5.03 (bs, 1H), 2.96 (s, 3H), 1.84 (d, J=6.76 Hz, 3H). LCMS: (ES) C12H13NO2 requires 203, found 204 [M+H]⁺. HPLC: Purity (λ 300 nm): 99.75%.

### Separation of Bk-5-MBPB

Bk-5-MBPB was Boc-protected. Next, isomeric separation of Boc-Bk-5-MBPB was conducted using the SFC and after chiral separation, both isomers of Boc-Bk-5-MBPB were deprotected to afford (−)-Bk-5-MBPB and (+)-Bk-5-MBPB. Each procedure is described below.

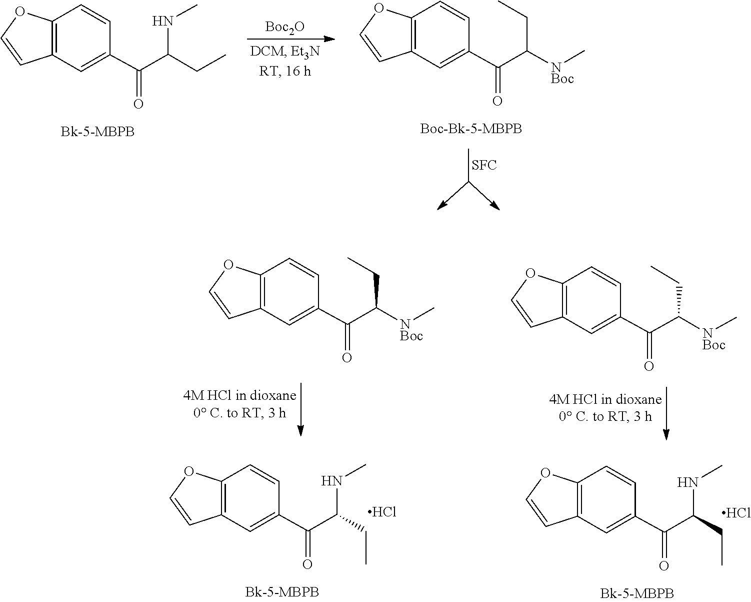

Synthesis of Boc-Bk-5-MBPB: To a stirred solution of 1-(benzofuran-5-yl)-2-(methylamino) butan-1-one (Bk-5-MBPB) (2.3 g, 10.59 mmol, 1 eq.) in dry DCM (30 ml) was added triethylamine (3.05 ml, 21.19 mmol, 2 eq.) and Boc anhydride (4.86 ml, 21.19 mmol, 2 eq.) and the resulting reaction mixture was allowed to stir at room temperature for 4 hours. Upon completion, (monitored by TLC, 10% EA in hexane), the reaction mixture was extracted with DCM (2×50 ml) and washed with water followed by brine solution. Combined organic solvent was dried over anhydrous sodium sulphate, solvent was evaporated under vacuum and purified by silica gel column chromatography using ethyl acetate/hexane (10:90 v/v) as eluent to get pure tert-butyl (1-(benzofuran-5-yl)-1-oxobutan-2-yl)(methyl)carbamate (Boc-Bk-5-MBPB) as a yellow sticky gum (1.7 g, 50%). ¹H NMR (400 MHz, CDCl₃) δ 8.38 (s, 1H), 8.03 (dd, J=8.76 Hz, 1H), 7.68 (m, 1H), 7.52 (d, J=4.8 Hz, 1H), 6.82 (s, 1H), 5.62 (m, 1H), 2.67 (s, 3H), 1.97 (m, 1H), 1.78 (m, 1H), 1.52 (s, 9H), 0.96 (m, 3H). Rotamer observed. LCMS: (ES) C₁₈H₂₃NO₄ requires 317, found 318 [M+H]⁺.

### Isomeric Separation by SFC:

Isomeric separation of Boc-Bk-5-MBPB was performed using SFC and the method of SFC separation is given below
Column: (R,R) Whelk-01 (4.5 mm×250 mm), 5p
Flow: 2 g/min
Mobile Phase: 75% CO2+25% (isopropanol)
ABPR: 100 bar
Temp: 35° C.
UV: 220 nm
Diluent: IPA

After SFC separation 1.6 g of Boc-Bk-5-MBPB-Isomer-1 and 1.5 g of Boc-Bk-5-MBPB-Isomer-2 were isolated. Characterization for both isomers is below:

Boc-Bk-5-MBPB-Isomer-1: ¹H NMR (400 MHz, CDCl₃) δ 8.38 (s, 1H), 8.03 (dd, J=8.56 Hz, 1H), 7.68 (d, J=8.28 Hz, 1H), 7.52 (d, J=8.40 Hz, 1H), 6.82 (s, 1H), 5.62 (q, 1H), 2.67 (s, 3H), 1.97 (m, 2H), 1.52 (s, 9H), 0.96 (m, 3H). Rotamer observed. LCMS: (ES) C₁₈H₂₃NO₄ requires 317, found 318 [M+H]⁺.

Boc-Bk-5-MBPB-Isomer-2: ¹H NMR (400 MHz, CDCl₃) δ 8.38 (s, 1H), 8.03 (dd, J=7.68 Hz, 1H), 7.68 (d, J=8.32 Hz, 1H), 7.52 (d, J=8.4 Hz, 1H), 6.81 (d, J=0.76 Hz, 1H), 5.62 (m, 1H), 2.67 (s, 4H), 1.96 (m, 3H), 1.52 (s, 9H), 0.98 (m, 4H). Extra peak present in aliphatic region. Rotamer observed. LCMS: (ES) C₁₈H₂₃NO₄ requires 317, found 318 [M+H]⁺.

Synthesis (−)-Bk-5-MBPB and (+)-Bk-5-MBPB: Both chiral intermediates were then subsequently de-protected using 4(M) HCl in 1,4 dioxane as described in Synthesis 18 to afford the two isomers of Bk-5-MBPB. Characterization of (−)-Bk-5-MBPB-isomer-1 and (+)-Bk-5-MBPB-isomer-2 are below:

(−)-Bk-5-MBPB: Following the deprotection, (−)-Bk-5-MBPB was afforded as a white solid (1.43 g, 99%). ¹HNMR(400 MHz, DMSO-d₆) δ 9.53 (s, 1H), 9.18 (s, 1H), 8.48 (s, 1H), 8.19 (d, J=2 Hz, 1H), 8.04 (dd, J=1.32 Hz, 8.68 Hz, 1H), 7.83 (d, J=8.68 Hz, 1H), 7.15 (d, J=1.04 Hz, 1H), 5.31 (s, 1H), 2.58 (s, 3H), 2.10 (m, 1H), 1.94 (m, 1H), 0.78 (t, J=7.44 Hz, 7.48 Hz, 3H). LCMS: (ES) C₁₃H₁₅NO₂ requires 217, found 218 [M+H]⁺. HPLC: Purity (λ 220 nm): 99.33%.

(+)-Bk-5-MBPB: Following the deprotection, (+)-Bk-5-MBPB was afforded as a white solid (1.33 g, 92%). ¹HNMR (400 MHz, DMSO-d₆) δ 9.50 (bs, 2H), 8.48 (d, J=0.84 Hz, 1H), 8.19 (d, J=1.9 Hz, 1H), 8.04 (d, J=8.6 Hz, 1H), 7.81 (d, J=8.64 Hz, 1H), 7.14 (d, J=1.1 Hz, 1H), 5.32 (s, 1H), 2.57 (s, 3H), 2.13 (m, 1H), 1.95 (m, 1H), 0.78 (t, J=7.4 Hz, 3H). LCMS: MS (ES) C₁₃H₁₅NO₂ requires 217, found 218 [M+H]⁺. HPLC: Purity (λ 220 nm): 98.15%.

### Separation of Bk-6-MBPB

Bk-6-MBPB was Boc-protected. Next, isomeric separation of Boc-Bk-6-MBPB was conducted using the SFC and after chiral separation, both isomers of Boc-Bk-6-MBPB were deprotected to afford (−)-Bk-6-MBPB and (+)-Bk-6-MBPB. Each procedure is described below.

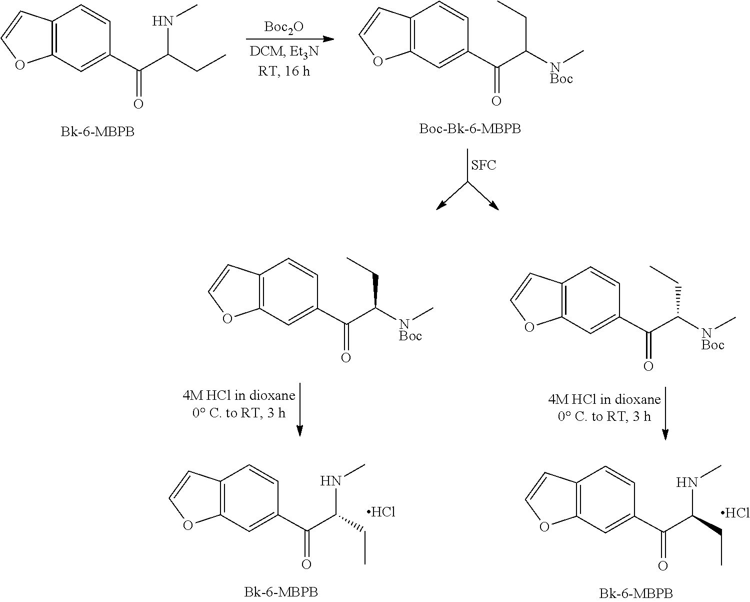

Synthesis of Boc-Bk-6-MBPB: To a stirred solution of 1-(benzofuran-6-yl)-2-(methylamino)butan-1-one (Bk-6-MBPB) (2.75 g, 12.65 mmol, 1eq.) in dry DCM (30 mL) was added triethylamine (3.65 mL, 25.31 mmol, 2 eq.) and Boc anhydride (5.8 mL, 25.31 mmol, 2 eq.) and the resulting reaction mixture was allowed to stir at room temperature for 4 hours. Upon completion of the reaction (monitored by TLC, 10% EA in hexane), the reaction mixture was extracted with DCM (2×50 ml) and washed with water followed by brine solution. The combined organic layers were dried over anhydrous sodium sulphate, solvent was evaporated under vacuum, and the crude material purified by silica gel column chromatography using ethyl acetate/hexane (10:90 v/v) as eluent to afford pure tert-butyl (1-(benzofuran-6-yl)-1-oxobutan-2-yl)(methyl)carbamate (Boc-Bk-6-MBPB) as yellow sticky gum (3.4 g, 84%). ¹H NMR (400 MHz, CDCl₃) δ 8.24 (s, 1H), 7.97 (dd, J=8.2 Hz, 1H), 7.76 (bs, 1H), 7.63 (bm, 1H), 6.80 (bs, 1H), 5.61 (t, J=5.64 Hz, 8.88 Hz, 1H), 2.66 (s, 3H), 1.99 (q, 2H), 1.55 (s, 9H), 0.98 (m, 3H). Rotamer observed. LCMS: (ES) C₁₈H₂₃NO₄ requires 317, found 318 [M+H]⁺.

### Isomer Separation by SFC

Isomer separation of Boc-Bk-6-MBPB was done by SFC and the method of SFC separation is given below:
Column: (R,R) Whelk-01 (4.5 mm×250 mm), 5p
Flow: 2 g/min
Mobile Phase: 80% CO2+25% (ISOPROPANOL)
ABPR: 100 bar
Temp: 35° C.
UV: 220 nm
Diluent: IPA

After SFC separation, 1 g of Boc-Bk-6-MBPB-Isomer-1 and 900 mg of Boc-Bk-6-MBPB-Isomer-2. Characterization for each isomer is given below:

Boc-Bk-6-MBPB-Isomer-1: ¹H NMR (400 MHz, CDCl₃) δ 8.25 (s, 1H), 7.97 (d, J=8.16 Hz, 1H), 7.89 (bs, 1H), 7.64 (m, 1H), 6.80 (s, 1H), 5.61 (q, 1H), 2.66 (s, 3H), 1.97 (m, 2H), 1.54 (s, 9H), 0.99 (m, 3H). Rotamers observed. LCMS: (ES) C₁₈H₂₃NO₄ requires 317, found 318 [M+H]⁺.

Boc-Bk-6-MBPB-Isomer-2: ¹H NMR (400 MHz, CDCl₃) δ 8.25 (s, 1H), 7.97 (d, J=8.08 Hz, 1H), 7.76 (s, 1H), 7.64 (m, 1H), 6.80 (s, 1H), 5.61 (m, 1H), 2.66 (s, 3H), 1.97 (m, 2H), 1.45 (s, 9H), 0.99 (m, 3H). Rotamers observed. LCMS: (ES) C₁₈H₂₃NO₄ requires 317, found 218 [M−Boc+H]⁺.

Synthesis (−)-Bk-6-MBPB and (+)-Bk-6-MBPB: Both chiral intermediates were then subsequently de-protected using 4(M) HCl in 1,4 dioxane as described in Synthesis 19 to afford the two isomers of Bk-6-MBPB. Characterization of (−)-Bk-6-MBPB-isomer-1 and (+)-Bk-6-MBPB-isomer-2 are below:

(−)-Bk-6-MBPB: Following the deprotection, (−)-Bk-6-MBPB was afforded as a white solid (1.4 g, 97%). ¹H NMR (400 MHz, CDCl₃) δ 10.69 (s, 1H), 9.03 (s, 1H), 8.15 (s, 1H), 7.87 (m, 2H), 7.72 (d, J=8.08 Hz, 1H), 6.86 (s, 1H), 5.00 (bs, 1H), 2.85 (s, 3H), 2.45 (m, 1H), 2.26 (m, 1H), 1.02 (t, J=7.48 Hz, 3H). LCMS: (ES) C₁₃H₁₅NO₂ requires 217, found 218 [M+H]⁺. HPLC: Purity (λ 220 nm): 99.14%.

(+)-Bk-6-MBPB: Following the deprotection, (+)-Bk-6-MBPB was afforded as an off-white solid (1.4 g, 97%). ¹H NMR (400 MHz, CDCl₃) δ 10.61 (s, 1H), 9.09 (s, 1H), 8.16 (s, 1H), 7.88 (m, 2H), 7.72 (d, J=8.08 Hz, 1H), 6.86 (s, 1H), 5.03 (bs, 1H), 2.95 (s, 3H), 2.45 (m, 1H), 2.24 (m, 1H), 1.02 (t, J=7.44 Hz, 3H). LCMS: (ES) C₁₃H₁₅NO₂requires 217, found 218 [M+H]⁺. HPLC: Purity (λ 220 nm): 99.44%.

### Alternative Synthesis of 6-MBPB

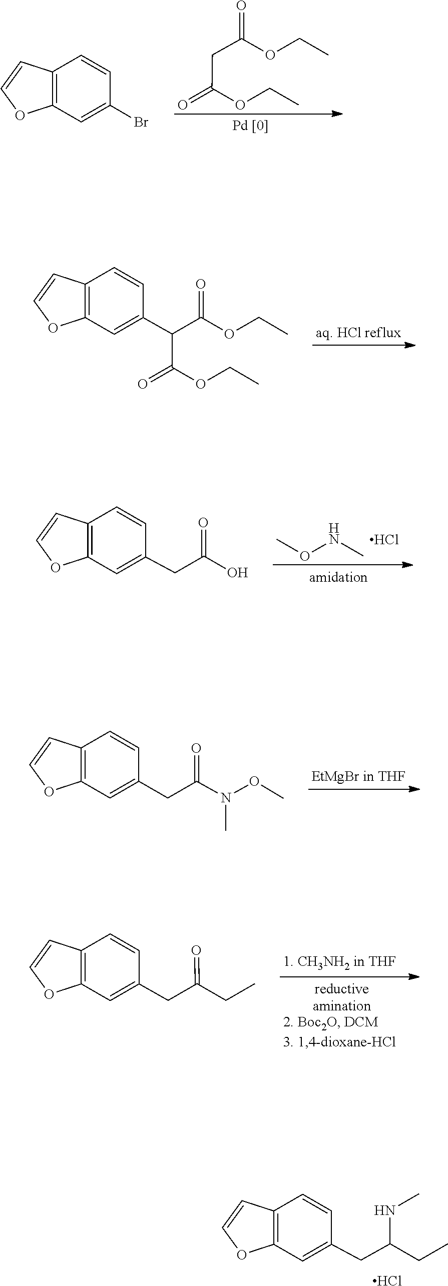

### Determination of Specific Rotation

Specific rotation was determined for individual enantiomers using a Jasco P-2000 Polarimeter, 589 nm Na lamp (Path Length 1 dm, 20° C. temperature, Concentration approx. 1 g/100 mL). EtOH was used as solvent for beta-ketone compounds, while distilled water was used for other compounds. Ten measurements were made for each compound.

| | Specific | Standard |
|---|---|---|
| Compound | rotation | Deviation |
| S 5-MAPB | 13.4 | 0.3 |
| R 5-MAPB | −14.3 | 0.2 |
| S-6-MAPB | 14.7 | 0.1 |
| R-6-MAPB | −14.3 | 0.2 |
| 5-MBPB enantiomer 1 | 18.9 | 0.5 |
| 5-MBPB enantiomer 2 | N/A | N/A |
| 6-MBPB enantiomer 1 | 19.6 | 0.9 |
| 6-MBPB enantiomer 2 | N/A | N/A |
| BK-5-MAPB Peak 1 | −53.6 | 0.1 |
| BK-5-MAPB Peak 2 | 56 | 0.2 |
| BK-6-MAPB Peak 1 | −49 | 0.2 |
| BK-6-MAPB Peak 2 | 47.1 | 0.2 |
| BK-5-MBPB Peak 1 | −15.6 | 0.2 |
| BK-5-MBPB Peak 2 | 14.1 | 0.2 |
| BK-6-MBPB Peak 1 | −6.7 | 0.1 |
| BK-6-MBPB Peak 2 | 5.6 | 0.2 |

### EXAMPLE 2: Synthesis of Select Compounds of the Present Invention

Methods for synthesis of the compounds described herein and/or starting materials are either described in the art or will be readily apparent to the skilled artisan in view of general references well-known in the art (see, e.g., Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the compounds of the invention.

Additional references include: Taniguchi et al. 2010. Journal of mass spectrometry, 45(12), 1473-1476; Shulgin & Shulgin. 1992. PiHKAL. A chemical love story, Transform Press, Berkeley CA; Glennon et al. 1986. J. Med. Chem., 29(2), 194-199; Nichols et al. 1991. J. Med. Chem., 34(1), 276-281; Kedrowski et al. 2007. Organic Letters, 9(17), 3205-3207; Heravi & Zadsirjan. 2016. Current Organic Synthesis, 13(6), 780-833; Keri et al. 2017. European J. Med. Chem., 138, 1002-1033; Perez-Silanes et al. 2001. J. Heterocyclic Chem, 38(5), 1025-1030; and references therein.

### Synthesis 1. 5-MBPB

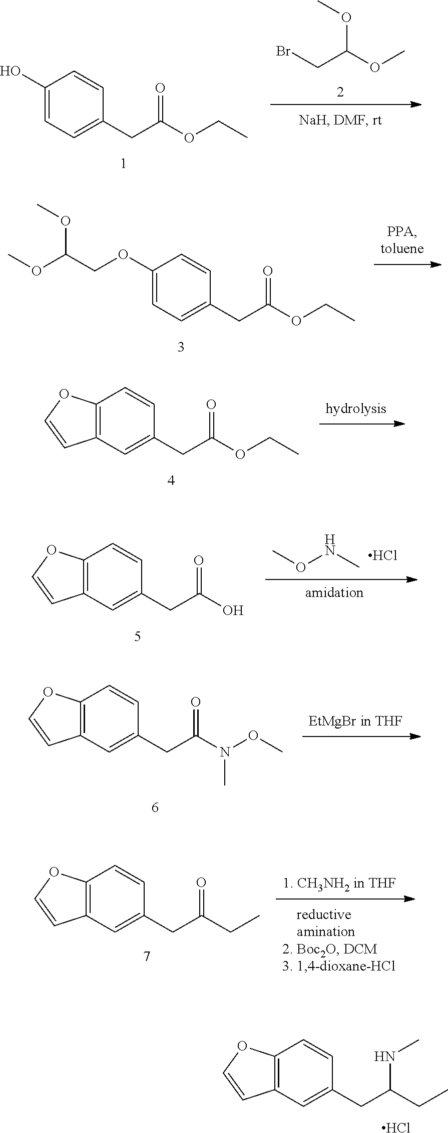

### Synthesis 2. 6-MBPB

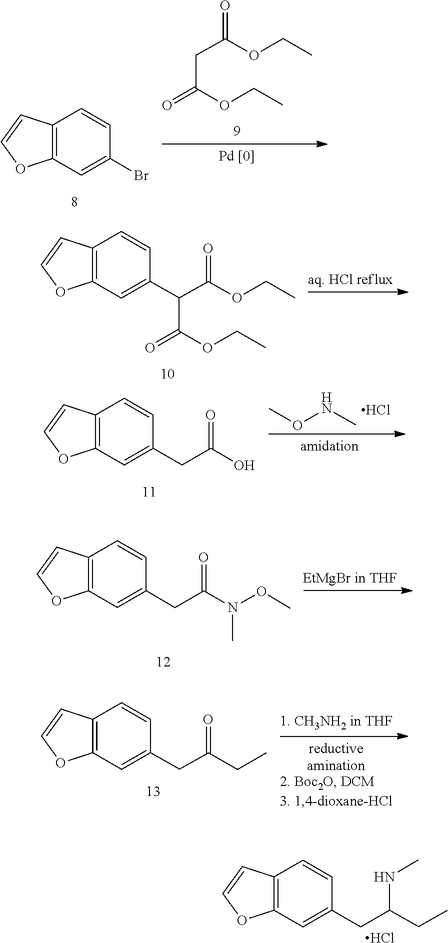

Other versions of these molecules can, for example, be synthesized following the methods of López and colleagues (López et al. 2012. British Journal of Pharmacology. 167 (2): 407-420). Additionally, the 5-MAPB and 6-MAPB can be made by analogy using the syntheses herein for 5-MBPB and 6-MBPB, using MeMgBr in THF in place of EtMgBr in THF in the third step.

### Synthesis 3. Bk-5-MAPB

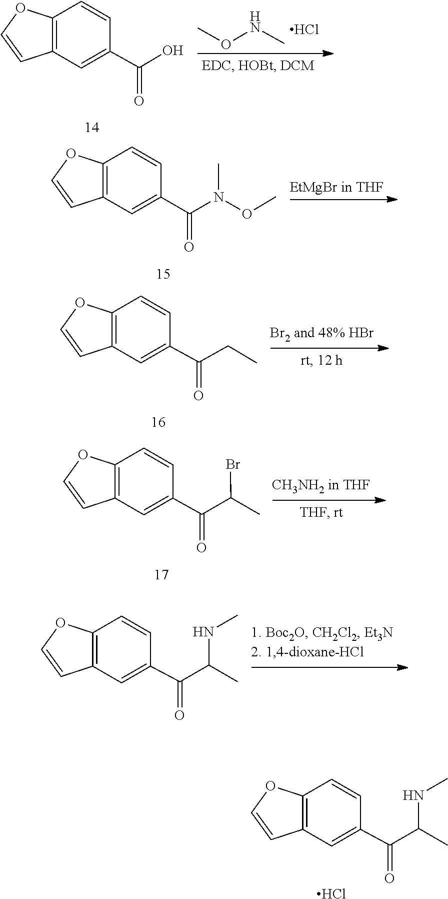

### Synthesis 4. Bk-5-MAPB

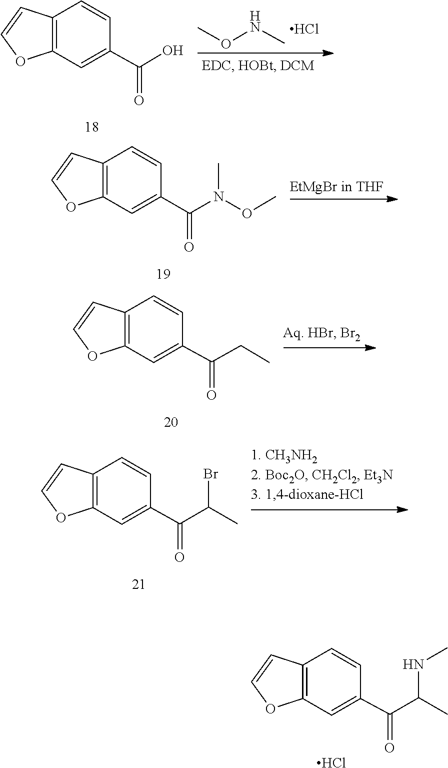

Other versions of these molecules can, for example, be synthesized following the methods of López and colleagues (López et al. 2012. British Journal of Pharmacology. 167 (2): 407-420). Additionally, the Bk-5-MBPB and Bk-66-MBPB can be made by analogy using the syntheses herein for Bk-5-MAPB and Bk-6-MAPB, using propyl magnesium bromide in THF in place of EtMgBr in THF in the second step.

### Synthesis 5. Alternative Method of Synthesis of Bk-5-MAPB and Bk-6-MAPB

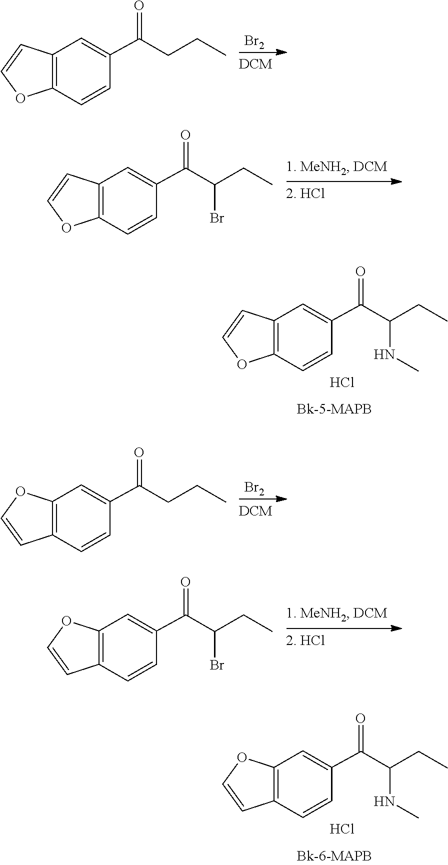

### Synthesis 6 Alternative Method of Synthesis Bk-5-MAPB and Rk-6-MAPB

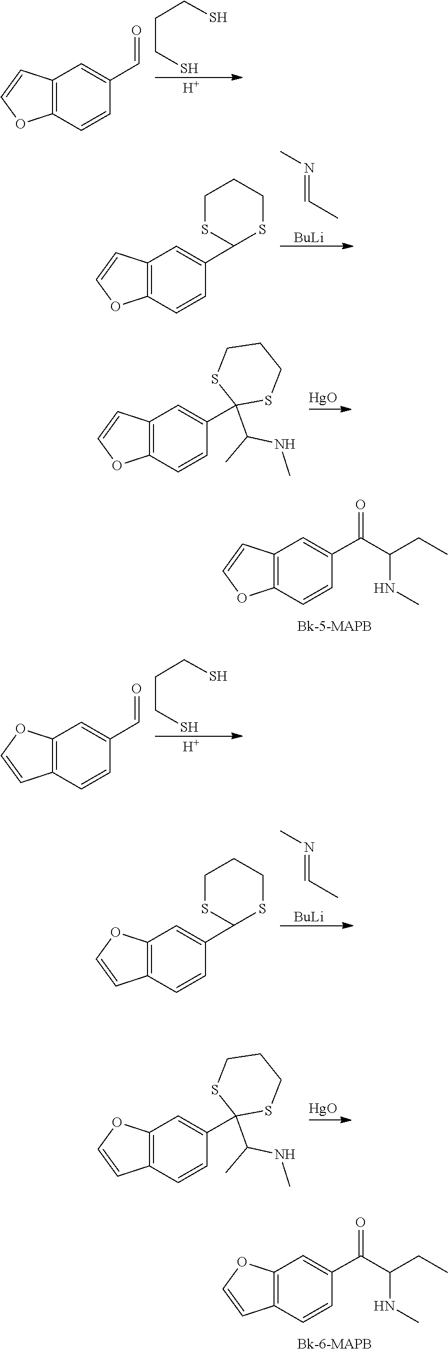

### Synthesis 7. Derivatization from Bk-5-MAPB

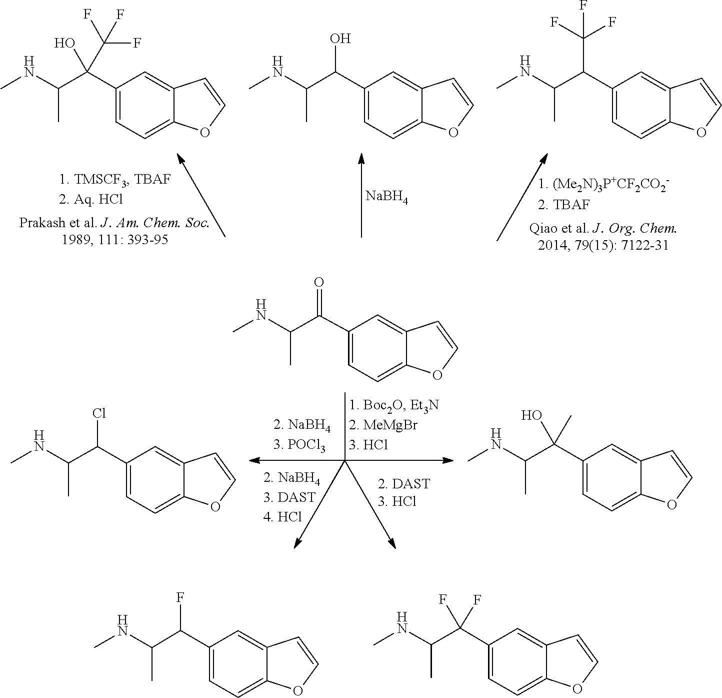

### Synthesis 8. Synthesis of 3-(benzofuran-6-yl)-N-methylbut-3-en-2-amine (Compound 1-4)

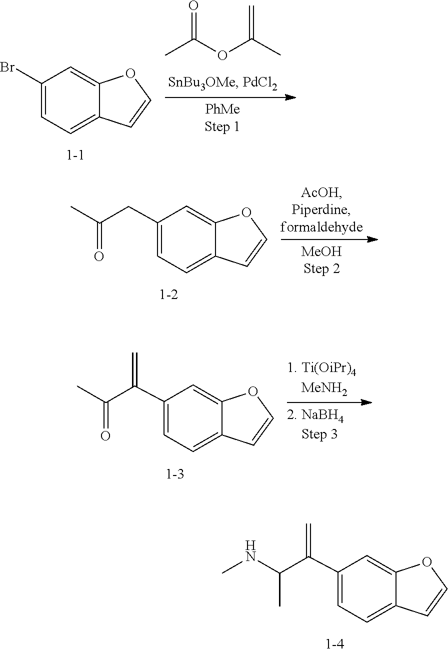

Step 1: A round-bottom flask is charged with 1-1, tributyltin methoxide, and palladium(II) chloride. The flask is then evacuated and refilled with anhydrous nitrogen three times before adding toluene and isopropenyl acetate. The reaction solution is then stirred with heating under nitrogen until the reaction is judged complete by TLC, HPLC, or other analytical method. Following the reaction, the mixture is cooled to room temperature, diluted with ethyl acetate, and washed three times with water. The organic layer is then dried over anhydrous Na₂SO₄, filtered, and concentrated to collect crude 1-2. This crude material can be taken to the next step without further purification or purified by standard techniques of the art to obtain the pure compound.

Step 2: A round-bottom flask is charged with 1-2, acetic acid, piperdine, and formaldehyde. Methanol is then added to dissolve the reaction components and the mixture is stirred until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude 1-3. This crude material can be taken to the next step without further purification or purified by standard techniques of the art to obtain the pure compound.

Step 3: In a round-bottom flask, 1-3, methylamine, and titanium (IV) isopropoxide are dissolved in ethanol and stirred under nitrogen. Once there is no remaining 1-3 as judged by TLC, HPLC, or other analytical method, the flask is opened briefly, and sodium borohydride is added slowly. The resulting slurry is stirred at room temperature overnight. Following the reaction, the mixture is diluted with ethyl acetate and washed three times with water. The organic layer is then dried over anhydrous Na₂SO₄, filtered, and concentrated to collect crude 1-4. This crude material can be purified by standard techniques of the art to obtain the pure compound.

The individual enantiomers of 1-4 can be separated using the methods described herein. For example, chiral SFC conditions are provided in Example 1. Following isolation of the pure enantiomers, they can be mixed again in any ratio necessary to obtain the desired effects,

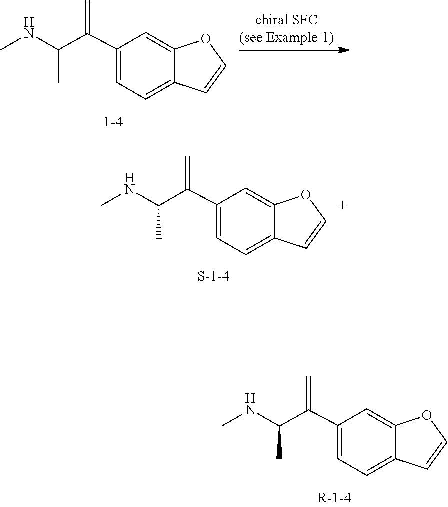

### Synthesis 9. Synthesis of 2-(benzofuran-6-yl)-3-(methylamino)butan-1-ol (Compound 2-6)

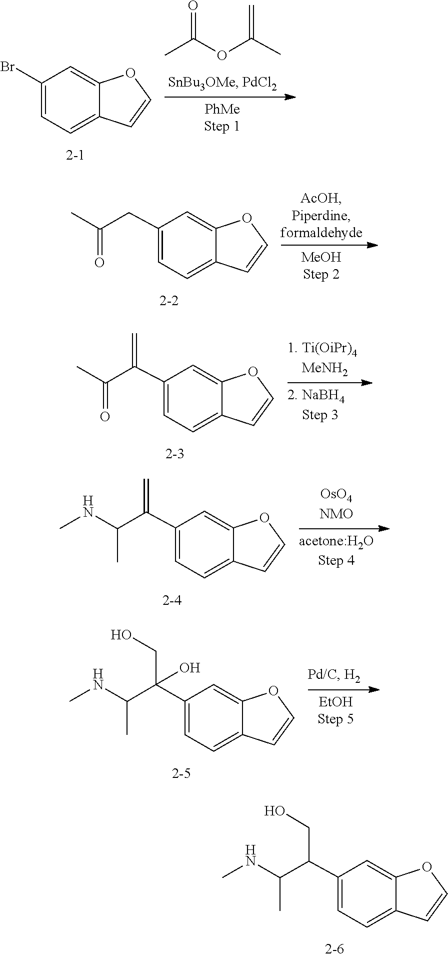

Step 1: A round-bottom flask is charged with 2-1, tributyltin methoxide, and palladium(II) chloride. The flask is then evacuated and refilled with anhydrous nitrogen three times before adding toluene and isopropenyl acetate. The reaction solution is then stirred with heating under nitrogen until the reaction is judged complete by TLC, HPLC, or other analytical method. Following the reaction, the mixture is cooled to room temperature, diluted with ethyl acetate, and washed three times with water. The organic layer is then dried over anhydrous Na₂SO₄, filtered, and concentrated to collect crude 2-2. This crude material can be taken to the next step without further purification or purified by standard techniques of the art to obtain the pure compound.

Step 2: A round-bottom flask is charged with 2-2, acetic acid, piperdine, and formaldehyde. Methanol is then added to dissolve the reaction components and the mixture is stirred until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude 2-3. This crude material can be taken to the next step without further purification or purified by standard techniques of the art to obtain the pure compound.

Step 3: In a round-bottom flask, 2-3, methylamine, and titanium (IV) isopropoxide are dissolved in ethanol and stirred under nitrogen. Once there is no remaining 2-3 as judged by TLC, HPLC, or other analytical method, the flask is opened briefly, and sodium borohydride is added slowly. The resulting slurry is stirred at room temperature overnight. Following the reaction, the mixture is diluted with ethyl acetate and washed three times with water. The organic layer is then dried over anhydrous Na₂SO₄, filtered, and concentrated to collect crude 2-4. This crude material can be purified by standard techniques of the art to obtain the pure compound.

Step 4: To a round-bottom flask containing 2-4 dissolved in acetone:H₂O is added NMO and a catalytic amount of osmium tetroxide. The resulting mixture is stirred at room temperature until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude 2-5. This crude material can be taken to the next step without further purification or purified by standard techniques of the art to obtain the pure compound.

Step 5: A round-bottom flask containing 2-5 and palladium on carbon is evacuated under vacuum and backfilled with nitrogen three times. Ethanol is then added to the flask and the resulting mixture is sparged with hydrogen gas while stirring. Once the nitrogen atmosphere is displaced by hydrogen, the reaction is stirred at room temperature until the reaction is judged complete by TLC, HPLC, or other analytical method. Following the reaction, the mixture is diluted with ethyl acetate, filtered through diatomaceous earth, and concentrated to collect crude 2-6. This crude material can be purified by standard techniques of the art to obtain the pure compound.

The individual enantiomers of 2-6 can be separated using the methods described herein. For example, chiral SFC conditions are provided in Example 1. Following isolation of the pure enantiomers, they can be mixed again in any ratio necessary to obtain the desired effects.

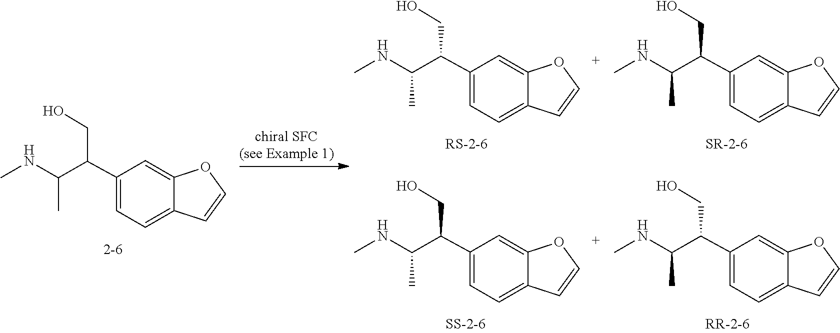

Alternatively, the diastereomers can first be separated by conventional, achiral purification techniques such as silica gel chromatography or preparative HPLC. The two purified diastereomers can then be further separated into the enantiomers as described.

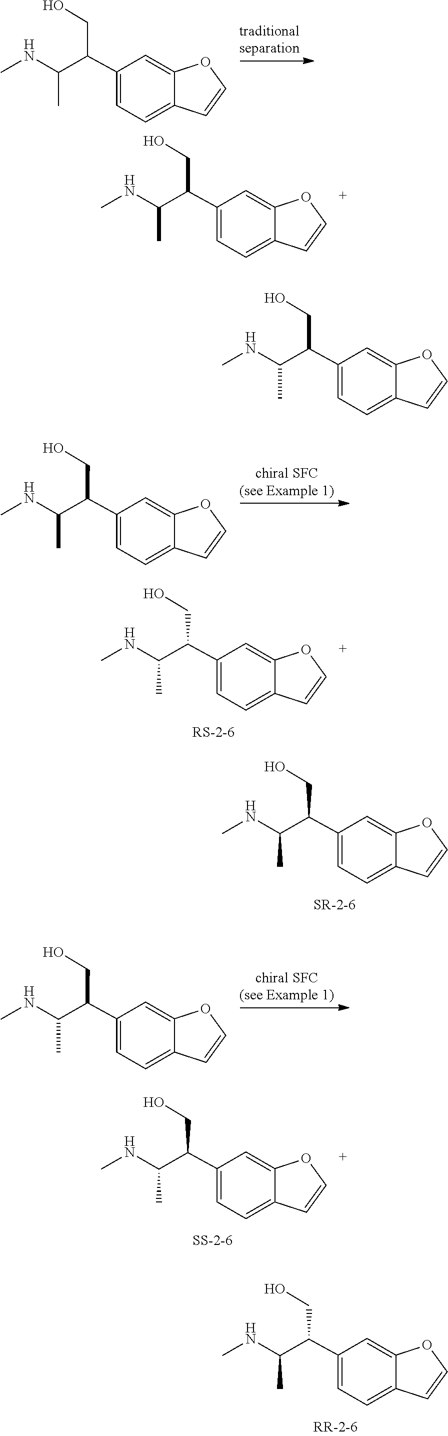

### Synthesis 10. Synthesis of 2-(benzofuran-6-yl)-1-cyclopropyl-N-ethylethan-1-amine (Compound 3-5)

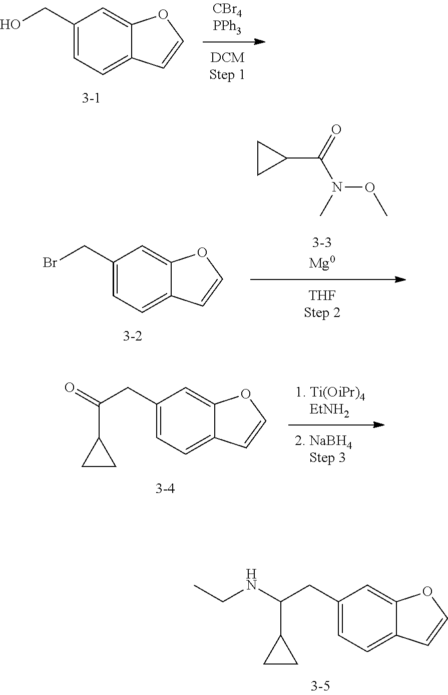

Step 1: To a round-bottom flask containing 3-1 dissolved in DCM is added triphenylphosphine and tetrabromomethane. The resulting mixture is stirred at room temperature until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude 3-2. This crude material can be purified by standard techniques of the art to obtain the pure compound.

Step 2: A round-bottom flask is charged with freshly activated magnesium metal then evacuated under reduced pressure and back-filled with nitrogen three times. Anhydrous THE is then added, and the reaction solution cooled to −78° C. followed by the slow addition of 3-2. Once reaction mixture ceases to self-heat, an anhydrous solution of 3-3 is added slowly. The resulting mixture is allowed to gradually warm to room temperature overnight. The reaction is then quenched under nitrogen using a saturated solution of aqueous NH₄Cl. The resulting mixture is then diluted with EtOAc, washed three times with water, dried over anhydrous Na₂SO₄, and filtered. The filtrate is then concentrated to collect crude 3-4. This crude material can be taken to the next step without further purification or purified by standard techniques of the art to obtain the pure compound.

Step 3: In a round-bottom flask, 3-4, ethylamine, and titanium (IV) isopropoxide are dissolved in ethanol and stirred under nitrogen. Once there is no remaining 3-4 as judged by TLC, HPLC, or other analytical method, the flask is opened briefly, and sodium borohydride is added slowly. The resulting slurry is stirred at room temperature overnight. Following the reaction, the mixture is diluted with ethyl acetate and washed three times with water. The organic layer is then dried over anhydrous Na₂SO₄, filtered, and concentrated to collect crude 3-5. This crude material can be purified by standard techniques of the art to obtain the pure compound.

The individual enantiomers of 3-5 can be separated using the methods described herein. For example, chiral SFC conditions are provided in Example 1. Following isolation of the pure enantiomers, they can be mixed again in any ratio necessary to obtain the desired effects.

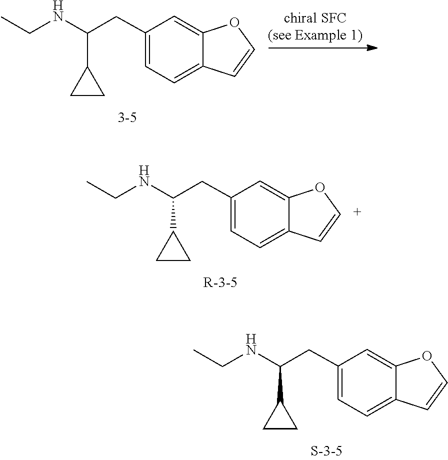 [SMILES: CCNC(Cc1ccc2ccoc2c1)C1CC1 ; CCN[C@H](Cc1ccc2ccoc2c1)C1CC1 ; CCN[C@@H](Cc1ccc2ccoc2c1)C1CC1]

### Synthesis 11. Synthesis of 3-(benzofuran-6-yl)-4-fluoro-2-(methylamino)butane-1,3-diol (Compound 4-8)

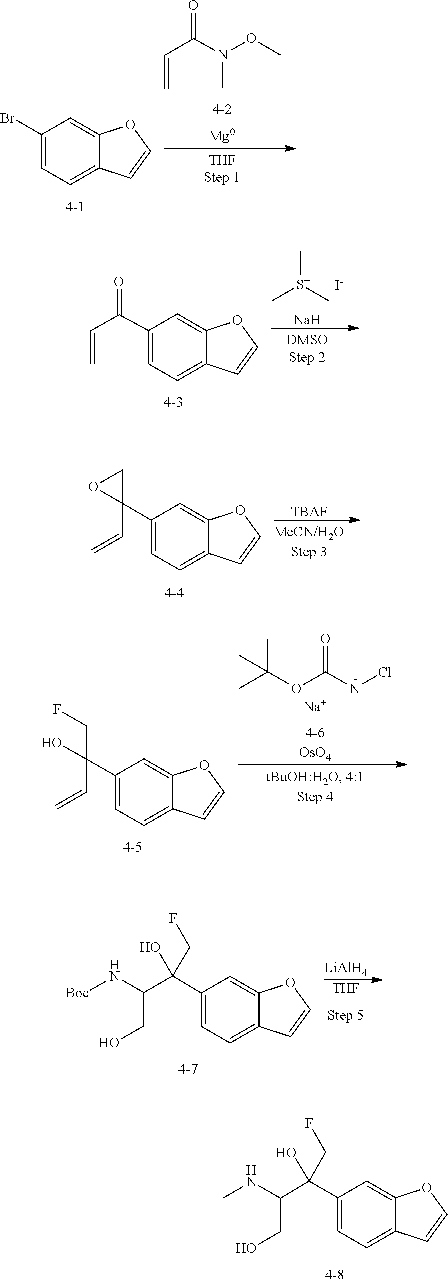

Step 1: A round-bottom flask is charged with freshly activated magnesium metal then evacuated under reduced pressure and back-filled with nitrogen three times. Anhydrous THE is then added, and the reaction solution cooled to −78° C. followed by the slow addition of 4-1. Once the reaction mixture ceases to self-heat, an anhydrous solution of 4-2 is added slowly. The resulting mixture is allowed to gradually warm to room temperature overnight. The reaction is then quenched under nitrogen using a saturated solution of aqueous NH₄Cl. The resulting mixture is then diluted with EtOAc, washed three times with water, dried over anhydrous Na₂SO₄, and filtered. The filtrate is then concentrated to collect crude 4-3. This crude material can be taken to the next step without further purification or purified by standard techniques of the art to obtain the pure compound.

Step 2: A round-bottom flask is charged with a stirbar, anhydrous DMSO, and trimethylsulfonium iodide. After evacuating the flask of ambient air and refilling with dry nitrogen three times, NaH is added slowly to the flask. Once the reaction solution has stopped giving off hydrogen gas, an anhydrous solution of 4-3 in DMSO is added slowly. The reaction is allowed to stir overnight and warm to room temperature. The reaction is then quenched under nitrogen using a saturated solution of aqueous NH₄Cl. The resulting mixture is then diluted with EtOAc, washed three times with water, dried over anhydrous Na₂SO₄, and filtered. The filtrate is then concentrated to collect crude 4-4. This crude material can be taken to the next step without further purification or purified by standard techniques of the art to obtain the pure compound.

Step 3: A round-bottom flask is charged with a stirbar, 4-4, and TBAF. The reagents are then dissolved in a solution of MeCN/H₂O, heated to just below reflux temperature, and stirred overnight. The reaction is monitored until completion by TLC, HPLC, or other analytical method. Following the reaction, the mixture is diluted with ethyl acetate and washed three times with water. The organic layer is then dried over anhydrous Na₂SO₄, filtered, and concentrated to collect crude 3-5. This crude material can be purified by standard techniques of the art to obtain the pure compound.

Step 4: A round-bottom flask is charged with a stirbar, 4-6, osmium tetroxide, and 4-5. The reagents are then dissolved in a solution of 4:1 tBuOH:H₂O. The resulting mixture is stirred at room temperature until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude a mixture of regio- and diastereoisomers of 4-7. This crude material can be purified by standard techniques of the art to obtain the pure compound.

Step 5: To a flame-dried round-bottom flask is added a stirbar, 4-7, and anhydrous THF. The resulting solution is cooled to −78° C. before adding LiAlH₄ slowly via syringe. The resulting mixture is allowed to slowly warm to room temperature and stirred until the reaction is judged complete by TLC, HPLC, or other analytical method. Following the reaction, the mixture is diluted with ether, slowly quenched with aqueous NaOH, then further quenched with water. The resulting slurry is diluted with EtOAc and washed three times with water. The organic layer is then dried over anhydrous Na₂SO₄, filtered, and concentrated to crude 4-8. This crude material can be purified by standard techniques of the art to obtain the pure compound.

The individual enantiomers of 4-8 can be separated using the methods described herein. For example, chiral SFC conditions are provided in Example 1. Following isolation of the pure enantiomers, they can be mixed again in any ratio necessary to obtain the desired effects.

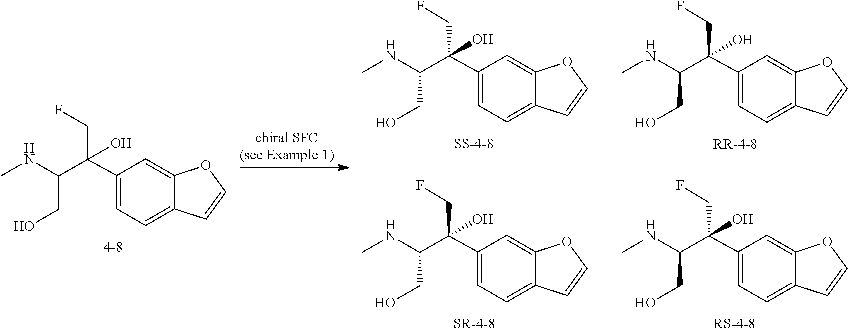

Alternatively, the diastereomers can first be separated by conventional, achiral purification techniques such as silica gel chromatography or preparative HPLC. The two purified diastereomers can then be further separated into the enantiomers as described.

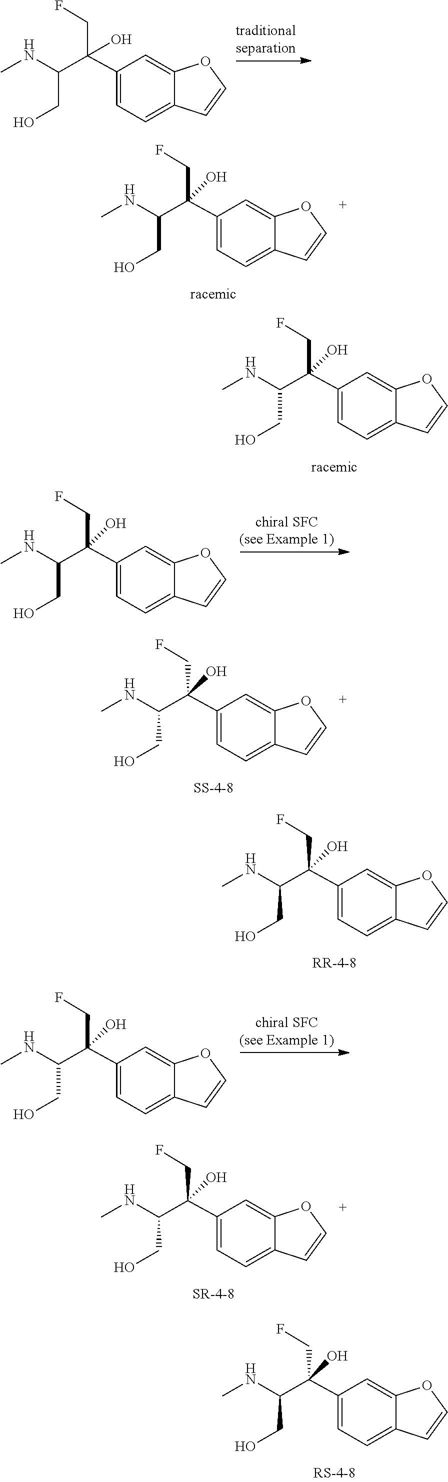

### Synthesis 12. Synthesis of 1-(benzofuran-5-yl)-N-methylpropan-2-amine (5-MAPB)

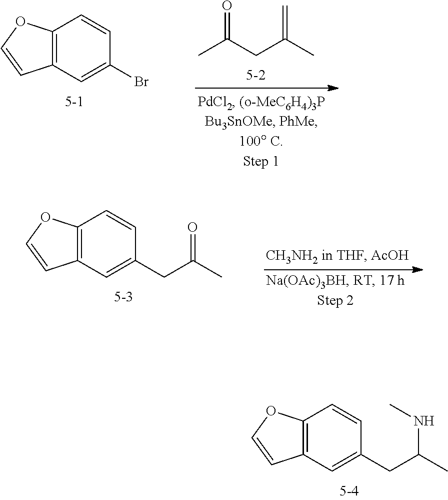

Step 1: To a stirred solution of 5-bromobenzofuran (5-1) (20 g, 101.52 mmol, 1 eq.) in dry toluene (400 mL) was added tri(o-tolyl)phosphine (1.84 g, 6.09 mmol, 0.06 eq.), tributyl tin methoxide (48.89 mL, 152.28 mmol, 1.5 eq.) and Isopropenyl acetate (16.99 mL, 156.34 mmol, 1.54 eq.) then the resulting reaction mixture was degassed under nitrogen for 15 minutes. Then palladium (II) chloride (1.26 g, 7.10 mmol, 0.07 eq.) was added to the reaction mixture and the resulting reaction mixture was heated to 100° C. for 16 h. Upon completion, monitored by TLC (10% EA in Hexane), the reaction mixture was cooled to RT, evaporate under vacuum. Then the residue was dissolved in ethyl acetate and filtered through celite bed, washed with water, and saturated potassium fluoride solution, followed by brine solution. Combined organic layer was dried over anhydrous sodium sulphate, solvent was removed under vacuum and purified by silica gel column chromatography using ethyl acetate/hexane (10:90 v/v) as eluent to afford 1-(benzofuran-5-yl)propan-2-one (5-3) as light yellow gum (17 g, 96%). ¹H NMR (400 MHz, DMSO-d₆) δ 7.96 (d, J=2.0 Hz, 1H), 7.53 (d, J=8.48 Hz, 1H), 7.46 (s, 1H), 7.13 (dd, J=1.52 Hz, 8.44 Hz, 1H), 6.92 (bs, 1H), 3.83 (s, 2H), 2.12 (s, 3H). LCMS: (ES) C₁₁H₁₀O₂ requires 174, found 175 [M+H]⁺.

Step 2: To a stirred solution of 1-(benzofuran-5-yl)propan-2-one (5-3) (16.0 g, 91.84 mmol, 1.0 eq.) in AcOH (70 ml) was added Methyl Amine (2M in THF) (230 mL, 460 mmol, 5 eq.) at RT and the resulting reaction mixture was stirred at RT for 1h. Then Na(OAc)₃BH (29.2 g, 137.77 mmol, 1.5 eq.) was added portion wise to the reaction mixture and continue to stir at RT for 16 h. After completion of reaction (TLC and LCMS) the reaction mixture was diluted with water (100 mL), and extracted with DCM (50 mL×2). Combined organic layer was dried over anhydrous sodium sulphate, solvent was removed under vacuum to got crude 1-(benzofuran-5-yl)-N-methylpropan-2-amine (5-MAPB) (16.0 g, 92%). 1H NMR(400 MHz, DMSO-d₆) δ 7.93 (s, 1H), 7.49 (d, J=8.36 Hz, 1H), 7.43 (s, 1H), 7.12 (d, J=7.56 Hz, 1H), 6.88 (s, 1H), 2.84-2.79 (m, 1H), 2.74-2.69 (m, 1H), 2.49 (bs, 1H), 2.94 (s, 3H), 0.91 (d, J=6.08 Hz, 3H). LCMS: (ES) C12H15NO requires 189, found 190 [M+H]⁺.

### Synthesis 13. Synthesis of 1-(benzofuran-6-yl)-N-methylpropan-2-amine (6-MAPB)

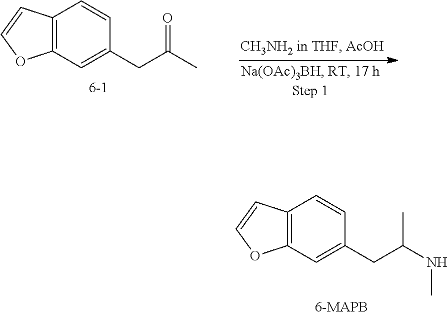

Step 1: To a stirred solution of 1-(benzofuran-6-yl)propan-2-one (6-1) (7 g, 40.23 mmol) in ACOH (15 mL), methyl amine (100 mL, 2M in methanol, 200 mmol) was added to it. After stirring for 15 mins, Na(OAc)₃BH (12.7 g, 60.34 mmol) was added to the reaction mixture and continue to stir at room temperature for 17 h. After the completion [Monitored with TLC, Mobile Phase 10% MeOH-DCM], the excess solvent was evaporated under reduced pressure and basified by sodium carbonate solution (30 mL) and extracted with DCM (2×50 mL). The obtained crude 1-(benzofuran-6-yl)-N-methylpropan-2-amine (6-MAPB) (7 g) was forwarded to the next step without further purification. ¹H NMR (400 MHz, DMSO-d₆): δ 7.90 (d, J=1.92 Hz, 1H), 7.54 (d, J=7.88 Hz, 1H), 7.39 (s, 1H), 7.08 (d, J=7.68 Hz, 1H), 6.89 (s, 1H), 2.85-2.80 (m, 1H), 2.74-2.65 (m, 2H), 2.28 (s, 3H), 0.91-0.85 (m, 3H). LCMS: (ES) C12H15NO requires 189.12, found 190.07 [M+H]⁺.

### Synthesis 14. Synthesis of 1-(benzofuran-5-yl)-N-methylbutan-2-amine (5-MBPB)

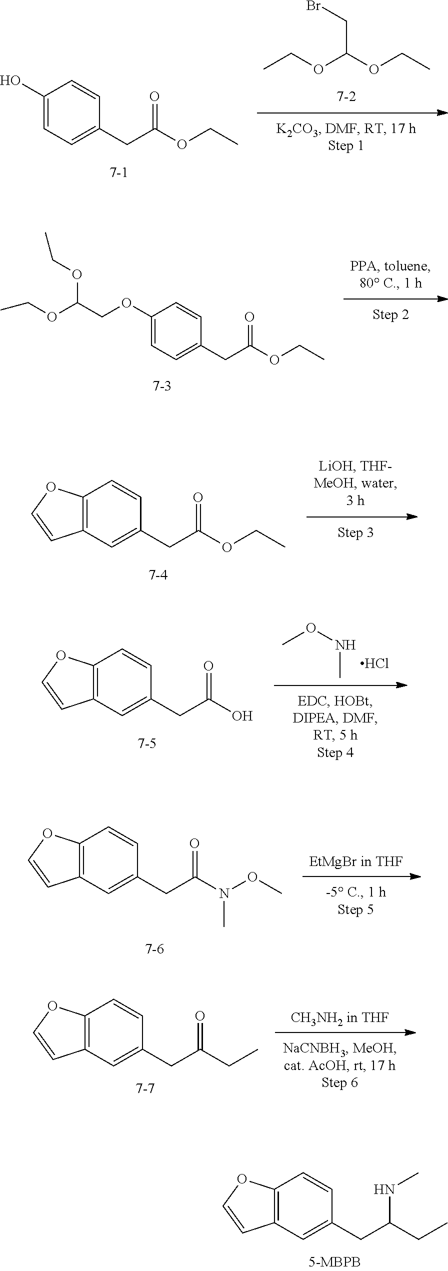

Step 1: To a stirred solution of ethyl 2-(4-hydroxyphenyl)acetate (7-1) (40 g, 222.22 mmol, 1.0 eq.) and 2-bromo-1,1-diethoxyethane (36.76 mL, 244.4 mmol, 1.1 eq.) in DMF (250 mL) was added K₂CO₃ (92 g, 666.66 mmol, 3.0 eq.) and heated to 100° C. for 17 h. After the completion [Monitored by TLC, mobile phase 10% EtOAc-Hexane], mixture was quenched with ice cold water (500 mL) and extracted with 30% ethyl acetate in hexane (1 L). Then the organic part was washed with saturated solution of NaCl, dried over anhydrous magnesium sulphate and concentrated under vacuum to afford the crude which was purified by silica gel (100-200 mesh) column chromatography eluted with 0-10% ethyl acetate in hexane to get the desired compound ethyl 2-(4-(2,2-diethoxyethoxy)phenyl)acetate (7-3) (20 g, 30%) as a colorless liquid. ¹H NMR (400 MHz, DMSO-d₆): δ 7.17 (d, J=8.56 Hz, 2H), 6.90 (d, J=8.52 Hz, 2H), 4.78 (t, J=5.2 Hz, 1H), 4.08 (m, 2H), 3.93 (d, J=5.2 Hz, 2H), 3.70-3.44 (m, 6H), 1.18-1.08 (m, 9H).

Step 2: To a stirred solution of ethyl 2-(4-(2,2-diethoxyethoxy)phenyl)acetate (7-3) (20 g, 74.62 mmol, 1.0 eq.) in toluene (100 mL) was added PPA (21.94 g, 223.8 mmol, 3.0 eq.) and heated to 80° C. for 3 h under nitrogen atmosphere. After the completion [Monitored with TLC, mobile phase 10% EtOAc-Hexane], reaction mixture was quenched with ice cold water (100 mL) and extracted with 30% ethyl acetate in hexane (300 mL). Then the organic part washed with saturated solution of NaCl, dried over anhydrous magnesium sulphate and concentrated under vacuum to afford the crude which was purified by silica gel (100-200 mesh) column chromatography eluted with 0-2% ethyl acetate in hexane to get the desired ethyl 2-(benzofuran-5-yl)acetate (7-4) (4.0 g, 26%) as a colorless liquid. ¹H NMR (400 MHz, DMSO-d₆): δ 7.97 (d, J=2.08 Hz, 1H), 7.54 (d, J=8.44 Hz, 2H), 7.20 (t, J=1.36 Hz, J=8.48 Hz, 1H), 6.93 (d, J=1.92 Hz, 1H), 4.10-4.04 (m, 2H), 3.73 (s, 2H), 1.17 (t, J=7 Hz, J=7.2 Hz, 3H).

Step 3: To a stirred solution of ethyl 2-(benzofuran-5-yl)acetate (7-4) (4 g, 19.6 mmol, 1.0 eq.) in THF (20 mL), MeOH (20 mL) was added followed by addition of lithium hydroxide (1.4 g, 58.82 mmol, 3.0 eq.) in water (20 mL). Reaction was stirred at RT for 2 hrs. After the completion [Monitored with TLC, Mobile Phase 60% EtOAc-Hexane], excess solvent was evaporated and acidified with 1(N) HCL in ice cooling condition and extracted with 10% MeOH in DCM. Organic part was washed with saturated solution of NaCl, dried over anhydrous magnesium sulphate and concentrated under vacuum to afford 2-(benzofuran-5-yl)acetic acid (7-5) (3.3 g, 95%) as an off white solid. ¹H NMR (400 MHz, DMSO-d₆): δ 12.28 (s, 1H), 7.96 (d, J=2.0 Hz, 1H), 7.52 (d, J=8.68 Hz, 2H), 7.20-7.18 (m, 1H), 6.92 (bs, 1H), 3.64 (s, 2H).

Step 4: To a stirred solution of 2-(benzofuran-5-yl)acetic acid (7-5) (3.3 g, 18.75 mmol, 1.0 eq.) in DMF (20 mL) were added DIPEA (9.8 mL, 56.25 mmol, 3.0 eq.),EDCI (3.93 g, 20.62 mmol, 1.1 eq.) and HOBT (3.79 g, 28.12 mmol, 1.5 eq.). Reaction was stirred at RT for 5 min followed by addition of weinreb amide (2 g, 20.62 mmol, 1.1 eq.). Reaction was stirred at RT for overnight. After the completion [Monitored with TLC, Mobile Phase 30% EtOAc-Hexane], reaction mixture was diluted with ethyl acetate (200 mL), washed 2-3 times with cold water. Organic phase was dried over magnesium sulphate and concentrated under reduced pressure to afford 2-(benzofuran-5-yl)-N-methoxy-N-methylacetamide (7-6) (4 g, 97%) as a light yellow sticky solid. ¹H NMR (400 MHz, DMSO-d₆): δ 7.95 (d, J=2.08 Hz, 1H), 7.51-7.49 (m, 2H), 7.18 (dd, J=1.36 Hz, 8.6 Hz, 1H), 6.91 (d, J=1.8 Hz, 1H), 3.80 (s, 2H), 3.67 (s, 3H), 3.11 (s, 3H).

Step 5: To a stirred solution of 2-(benzofuran-5-yl)-N-methoxy-N-methylacetamide (7-6) (4 g, 18.26 mmol, 1.0 eq.) in THE (20 mL), ethyl magnesium bromide (1 M, 27.39 mL, 27.39 mmol, 1.5 eq.) was added drop wise at 0° C. under nitrogen atmosphere. The reaction mixture was stirred at 0° C. for 1 hr. After completion [Monitored with TLC, mobile Phase 10% EtOAc-Hexane], it was quenched by saturated ammonium chloride solution (5 mL) and extracted with ethyl acetate (50 mL) and washed with NaCl solution. Organic phase was dried over magnesium sulphate and concentrated under reduced pressure. Crude compound was purified by silica gel (100-200 mesh) column chromatography eluted with 10-20% ethyl acetate in hexane to afford the desired 1-(benzofuran-5-yl)butan-2-one (7-7) (3.2 g, 93%) as a yellow liquid. ¹H NMR (400 MHz, DMSO-d₆): δ 7.96 (d, J=1.92 Hz, 1H), 7.52 (d, J=8.4 Hz, 1H), 7.46 (s, 1H), 7.12 (d, J=7.36 Hz, 1H), 6.91 (bs, 1H), 3.82 (s, 2H), 2.53 (m, 2H), 0.91 (t, J=7.24 Hz, J=7.28 Hz, 3H).

Step 6: To a stirred solution of 1-(benzofuran-5-yl)butan-2-one (7-7) (3.2 g, 17.02 mmol, 1.0 eq) and methanol (20 mL), methyl amine (43 mL, 2M in methanol, 85.1 mmol, 5.0 eq) was added followed by addition of catalytic amount of AcOH (0.5 mL). After stirring for 15 mins, NaCNBH3 (3.2 g, 51.06 mmol, 3.0 eq) was added. The resultant mixture was stirred at room temperature for 17 h. After the completion [Monitored with TLC, Mobile Phase 5% MeOH-EtOAc, Rf-0.2], the excess solvent was evaporated under reduced pressure and basified by sodium carbonate solution (30 mL) and extracted with DCM (2×100 mL). The obtained crude 1-(benzofuran-5-yl)-N-methylbutan-2-amine (5-MBPB) (3.3 g, 95%). ¹H NMR (400 MHz, DMSO-d₆): δ 7.94-7.91 (m, 1H), 7.50-7.46 (m, 2H), 7.15 (d, J=8.4 Hz, 1H), 6.89 (d, J=1.72 Hz, 1H), 2.82-2.61 (m, 3H), 2.32 (s, 3H), 1.40-1.30 (m, 2H), 0.95-0.75 (m, 3H). LCMS: (ES) C13H17NO requires 203, found 204 [M+H].

### Synthesis 15. Synthesis of 1-(benzofuran-6-yl)-N-methylbutan-2-amine (6-MBPB)

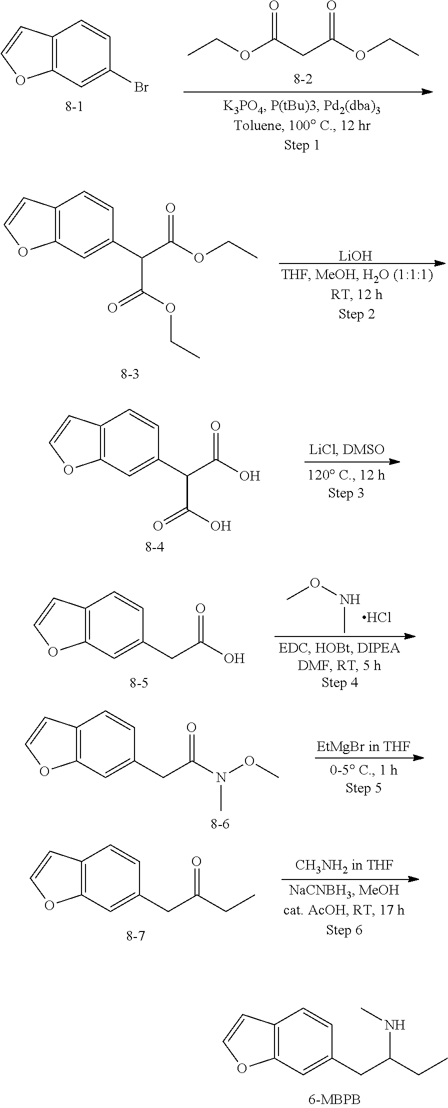

Step 1: A solution of diethyl malonate (8-2) (20.42 mL, 134.01 mmol, 1.1 eq.) and K ₃PO₄(51.65 g, 243.65 mmol, 2 eq.) in toluene (120 mL) was purged with nitrogen for 10 min. Then P(tBu)₃ (12.45 g, 24.36 mmol, 0.2 eq.) was added to the reaction mixture followed by 6-bromobenzofuran (8-1) (24 g, 121.82 mmol, 1.0 eq.) and Pd₂(dba)₃ (2.31 g, 2.43 mmol, 0.02 eq.). Reaction mixture was stir at RT and continue at 100° C. for 12 h. After completion of reaction monitored by TLC and LCMS, the mixture was cooled to room temperature and concentrated under reduced pressure. Then the reaction mixture was diluted with water [500 mL] and extracted with EtOAc [500 mL×2]. Organic layer was separated, dried over sodium sulphate and concentrated under vacuum. Then the crude was purified by silica gel (100-200 mesh) column chromatography eluted with 0-10% ethyl acetate in hexane to afford diethyl 2-(benzofuran-6-yl)malonate (8-3) (15 g, 44%) as a colorless liquid. ¹H NMR (400 MHz, DMSO-d₆): δ 8.01 (d, J=2.12 Hz, 1H), 7.63 (t, J=8.04 Hz, J=7.44 Hz, 2H), 7.28-7.26 (m, 1H), 6.96 (bs, 1H), 5.07 (s, 1H), 4.21-4.08 (m, 4H), 1.20-1.15 (m, 6H). LCMS: (ES) C15H16O5 requires 276, found 277 [M+H]⁺.

Step 2: To a stirred solution of diethyl 2-(benzofuran-6-yl)malonate (8-3) (15 g, 54.34 mmol, 1.0 eq.) in THE (50 mL), MeOH (50 mL) was added followed by addition of lithium hydroxide (5.7 g, 135.87 mmol, 2.5 eq.) in water (50 mL). Then the reaction was stir at RT for 12 h. After the completion [Monitored by TLC, mobile Phase 5% MeOH-DCM], excess solvent was evaporated and acidified with 1(N) HCL in ice cooling condition and extracted with 10% MeOH in DCM. Organic part was washed with saturated solution of NaCl, dried over anhydrous magnesium sulphate and concentrated under vacuum to afford 2-(benzofuran-6-yl)malonic acid (8-4) (11.5 g, 96%) as an off white solid. ¹H NMR (400 MHz, DMSO-d₆): δ 12.71 (s, 2H), 7.99 (d, J=2.08 Hz, 1H), 7.62-7.58 (m, 2H), 7.29 (d, J=14.68 Hz, 1H), 6.95 (d, J=1.84 Hz, 1H). LCMS: (ES) C11H8O5 requires 220, found 219 [M−H]⁺.

Step 3: To a stirred solution of 2-(benzofuran-6-yl)malonic acid (8-4) (11.5 g, 52.27 mmol, 1.0 eq) in DMSO (50 mL) were added LiCl (4.39 g, 104.54 mmol, 2.0 eq) and H₂O (5 mL) heated to 120° C. temperature for 12 hrs. After completion [Monitored with TLC, Mobile Phase 100% EtOAc, Rf-0.6], reaction mixture was diluted with water [250 mL] and extracted with EtOAc [500 mL×2]. Then the organic layer was extracted and dried over magnesium sulphate and concentrated under vacuum to afford 2-(benzofuran-6-yl)acetic acid (8-5) (9 g, 97.73%) as an off white solid crude. ¹H NMR (400 MHz, DMSO-d₆): δ 12.03 (s, 1H), 7.95 (d, J=2.0 Hz, 1H), 7.58 (d, J=7.92 Hz, 1H), 7.48 (s, 1H), 7.16 (d, J=7.88 Hz, 1H), 6.92 (d, J=0.92 Hz, 1H), 3.68 (s, 2H).

Step 4: To a stirred solution of 2-(benzofuran-6-yl)acetic acid (8-5) (9.0 g, 51.13 mmol, 1.0 eq.) in DMF (15 mL) were added DIPEA (26.74 mL, 153.40 mmol, 3.0 eq.), EDCI (10.74 g, 56.25 mmol, 1.1 eq.) and HOBT (8.62 g, 63.92 mmol, 1.5 eq.). The reaction mixture was stirred at RT for 5 min followed by addition of weinreb amide (5.45 g, 56.25 mmol, 1.1 eq.), then it was stir at RT for 5 h. After the completion [monitored by TLC, mobile Phase 30% EtOAc-hexane], reaction mixture was diluted with ethyl actate (500 mL), washed 2-3 times with cold water and dried over magnesium sulphate and concentrated under reduced pressure to afford 2-(benzofuran-6-yl)-N-methoxy-N-methylacetamide (8-6) (8.0 g, 71%) as a light yellow sticky solid. ¹H NMR (400 MHz, DMSO-d₆): δ 7.94 (d, J=2.04 Hz, 1H), 7.57 (d, J=7.92 Hz, 1H), 7.45 (s, 1H), 7.13 (d, J=7.96 Hz, 1H), 6.91 (bs, 1H), 3.83 (s, 2H), 3.68 (s, 3H), 3.11 (s, 3H). LCMS: (ES) C12H13NO3 requires 219, found 220 [M+H]⁺.

Step 5: To a stirred solution of 2-(benzofuran-6-yl)-N-methoxy-N-methylacetamide (8-6) (8.0 g, 36.53 mmol, 1.0 eq.) in THE (50 mL), ethyl magnesium bromide (1 M, 54.79 mL, 54.79 mmol, 1.5 eq.) was added drop wise at 0° C. under nitrogen atmosphere. The reaction mixture was stirred at 0° C. for 1 h. After completion [monitored by TLC, mobile Phase 10% EtOAc-hexane], it was quenched by saturated ammonium chloride solution (5 mL) and extracted with ethyl acetate (100 mL) and washed with NaCl solution then dried over magnesium sulphate and concentrated under reduced pressure. The crude compound was purified by silica gel (100-200 mesh) column chromatography eluted with 10-20% ethyl acetate in hexane to afford 1-(benzofuran-6-yl)butan-2-one (8-7) (6.0 g, 87%) as a yellow liquid. ¹H NMR (400 MHz, DMSO-d₆): δ 7.94 (d, J=2.16 Hz, 1H), 7.58 (d, J=7.92 Hz, 1H), 7.42 (s, 1H), 7.08 (d, J=8.0 Hz, 1H), 6.92 (t, J=0.76 Hz, J=1.12 Hz, 1H), 3.85 (s, 1H), 2.54-2.49 (m, 2H), 0.91 (t, J=7.2 Hz, 3H). LCMS: (ES) C12H₁₂O₂ requires 188, found 189 [M+H]⁺.

Step 6: To a stirred solution of 1-(benzofuran-6-yl)butan-2-one (8-7) (6.0 g, 31.91 mmol, 1.0 eq.) in methanol (30 mL), methyl amine (79.78 mL, 2M in methanol, 159.57 mmol, 5.0 eq.) was added followed by the addition of catalytic amount of AcOH (1.0 mL). After stirring for 15 min, NaCNBH₃ (56.03 g, 95.74 mmol, 3.0 eq.) was added to it. The resultant mixture was stirred at room temperature for 17 h. After completion [monitored by TLC, mobile Phase 10% MeOH-EtOAc], the excess solvent was evaporated under reduced pressure and basified by sodium carbonate solution (60 mL) then extracted with DCM (2×200 mL). Then dried over magnesium sulphate and concentrated under reduced pressure to obtained crude 1-(benzofuran-6-yl)-N-methylbutan-2-amine (6-MBPB) (5.0 g, 77%) which was forwarded to the next step without purification. ¹H NMR (400 MHz, DMSO-d₆): δ 7.90 (d, J=2.08 Hz, 1H), 7.54 (d, J=7.88 Hz, 1H), 7.40 (s, 1H), 7.09 (d, J=7.8 Hz, 1H), 6.89 (d, J=1.08 Hz, 1H), 2.77-2.72 (m, 1H), 2.67-2.62 (m, 1H), 2.58-2.53 (m, 1H), 2.26 (s, 3H), 1.35-1.23 (m, 2H), 0.84 (t, J=7.36 Hz, J=7.40 Hz, 3H). LCMS: (ES) C13H17NO requires 203, found 204.43 [M+H]⁺.

### Synthesis 16. Synthesis of Bk-5-MAPB HCl

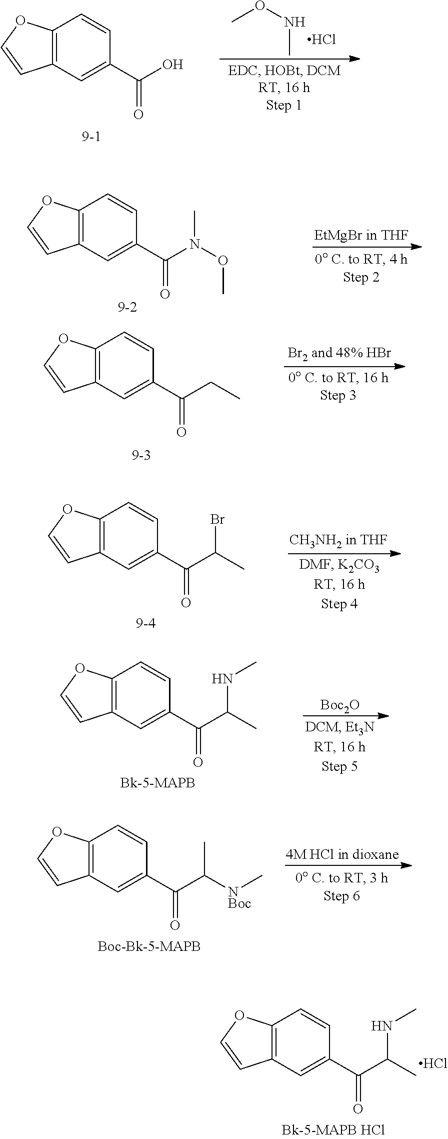

Step 1: Synthesis of N-Methoxy-N-methylbenzofuran-5-carboxamide (9-2): To a stirred solution of benzofuran-5-carboxylic acid (9-1) (10 g, 61.72 mmol, 1 eq.) in dry DCM (100 ml) was added DIPEA (32 ml, 185.18 mmol, 3 eq.) followed by EDC.HCl (13 g, 67.90 mmol, 1.1 eq.) and HOBT (12.5 g, 92.59 mmol, 1.5 eq.) under N₂ atmosphere at room temperature and the resulting reaction mixture was allowed to stir at room temperature for 15 minutes. Then, N, O-dimethylhydroxylamine hydrochloride (6.62 g, 67.90 mmol, 1.1 eq.) was added to the resulting reaction mixture and was allowed to stir at room temperature for 16 hours. Completion of the reaction was monitored by TLC (20% EA in hexane). Upon completion, the reaction mixture was extracted with DCM twice (2×200 ml) and washed with water followed by brine solution. The combined organic layers were dried over anhydrous sodium sulphate, solvent was removed under vacuum and purified by silica gel column chromatography using ethyl acetate/hexane (20:80 v/v) as eluent to afford pure N-methoxy-N-methylbenzofuran-5-carboxamide (9-2) as yellow sticky gum (10.6 g, 83%). ¹H NMR (400 MHz, CDCl₃) δ 7.97 (s, 1H), 7.66 (m, 2H), 7.50 (d, J=8.56 Hz, 1H), 6.80 (d, J=1.08 Hz, 1H), 3.54 (s, 3H), 3.37 (s, 3H). LCMS: (ES) C₁₁H₁₁NO₃ requires 205, found 206 [M+H]⁺.

Step 2: Synthesis of 1-(Benzofuran-5-yl) propan-1-one (9-3): To a stirred solution of N-methoxy-N-methylbenzofuran-5-carboxamide (9-2) (14 g, 68.22 mmol, 1 eq.) was added dry THE (250 ml) at 0° C. and was added 3 (M) solution of EtMgBr in diethyl ether (45 ml, 136.44 mmol, 2 eq.) to the reaction mixture and allowed to stir at room temperature for 4 hours. Upon completion of reaction (monitored by TLC, 20% EA in hexane) was quenched with saturated NH₄Cl solution and extracted with ethyl acetate, twice (2×100 ml), then washed with water followed by brine solution. The combined organic layers were dried over anhydrous sodium sulphate, solvent was evaporated under vacuum to afford crude compound 1-(benzofuran-5-yl) propan-1-one (9-3) as yellow solid (10 g, 84%). ¹H NMR (400 MHz, CDCl₃) δ 8.25 (d, J=1.48 Hz, 1H), 7.97 (dd, J=1.72 Hz, 8.72 Hz, 1H), 7.67 (d, J=6.68 Hz, 1H), 7.53 (d, J=8.72 Hz, 1H), 6.84 (d, J=1.56 Hz, 1H), 3.08 (q, 2H), 1.24 (t, J=7.24 Hz, 3H). LCMS: (ES) C₁₁H₁₀O₂ requires 174, found 175 [M+H]⁺.

Step 3: Synthesis of 1-(Benzofuran-5-yl)-2-bromopropan-1-one (9-4): To a stirred solution of 1-(benzofuran-5-yl)propan-1-one (9-3) (9 g, 51.66 mmol, 1 eq.) in dry THE (90 ml) was added hydrobromic acid 48% in water (133 ml, 1653.27 mmol, 32 eq.) and bromine (2.91 ml, 56.83 mmol, 1.1 eq.) dropwise at 0° C. and the reaction mixture was allowed to stir at room temperature for 16 hours. Upon completion, the reaction mixture (monitored by TLC, 10% EA in hexane) was quenched with saturated sodium carbonate solution, extracted with ethyl acetate (2×100 ml), and washed with water and brine solution. The combined organic layers were dried over anhydrous sodium sulphate, solvent was evaporated under vacuum and purified by silica gel column chromatography using ethyl acetate/hexane (10:90 v/v) as eluent to afford pure compound 1-(benzofuran-5-yl)-2-bromopropan-1-one (9-4) as yellow sticky gum (9 g, 68%). ¹H NMR (400 MHz, CDCl₃) δ 8.32 (d, J=1.52 Hz, 1H), 8.02 (dd, J=1.76 Hz, 8.72 Hz, 1H), 7.69 (d, J=2.2 Hz, 1H), 7.57 (d, J=8.72 Hz, 1H), 6.86 (d, J=1.96 Hz, 1H), 5.39 (q, 1H), 1.93 (t, J=6.6 Hz, 3H). LCMS: (ES) C₁₁H₁₉BrO₂ requires 253, found 254 [M+H]⁺.

Step 4: Synthesis of 1-(Benzofuran-5-yl)-2-(methylamino) propan-1-one (9-5): To a stirred solution of 1-(benzofuran-5-yl)-2-bromopropan-1-one (9-4) (9 g, 35.57 mmol, 1eq.) in dry DMF (90 ml) was added potassium carbonate (7.36 g, 53.36 mmol, 1.5eq.) and methyl amine 2(M) in THE (106.5 ml, 213.43 mmol, 6eq.) in a sealed round bottom flask and the resulting reaction mixture was allowed to stir at room temperature for 16 hours. Upon completion of reaction (monitored by TLC, 10% EA in hexane) the crude was extracted with ethyl acetate (2×100 ml), and washed with water (2×100 ml) and brine solution. The combined organic solvent was dried over anhydrous sodium sulphate and solvent was evaporated under vacuum to afford crude 1-(benzofuran-5-yl)-2-(methylamino) propan-1-one (9-5) as yellow sticky gum (5.4 g, 74%). ¹H NMR (400 MHz, CDCl₃) δ 8.27 (s, 1H), 7.98 (dd, J=1.52 Hz, 8.68 Hz, 1H), 7.69 (d, J=2 Hz, 1H), 7.57 (d, J=8.56 Hz, 1H), 6.86 (s, 1H), 4.31 (q, 1H), 2.38 (s, 3H), 1.33 (d, J=7 Hz, 3H). LCMS: (ES) C₁₂H₁₃NO₂ requires 203, found 204 [M+H]⁺.

Step 5: Synthesis of tert-Butyl (1-(benzofuran-5-yl)-1-oxopropan-2-yl) (methyl) carbamate (Boc-Bk-5-MAPB): To a stirred solution of 1-(benzofuran-5-yl)-2-(methylamino) propan-1-one (9-5) (5.2 g, 25.61 mmol, 1eq.) in dry DCM (50 ml) was added triethylamine (7.39 ml, 51.23 mmol, 2eq.) and Boc anhydride (11.75 ml, 51.23 mmol, 2 eq.) and the resulting reaction mixture was allowed to stir at room temperature for 4 hours. Upon completion of reaction (monitored by TLC, 10% EA in hexane), the reaction mixture was extracted with DCM (2×100 ml) and washed with water followed by brine solution. Combined organic solvent was dried over anhydrous sodium sulphate and solvent was evaporated under vacuum and purified by silica gel column chromatography using ethyl acetate/hexane (10:90 v/v) as eluent to afford pure tert-butyl (1-(benzofuran-5-yl)-1-oxopropan-2-yl)(methyl)carbamate (Boc-Bk-5-MAPB) as yellow sticky gum (3.9 g, 50%). ¹H NMR (400 MHz, CDCl₃) δ 8.33 (s, 1H), 7.99 (d, J=8.52 Hz, 1H), 7.66 (bs, 1H), 7.52 (d, J=8.56 Hz, 1H), 6.81 (d, J=1.12 Hz, 1H), 5.80 (q, 1H), 2.59 (s, 3H), 1.43 (s, 9H), 1.37 (m, 3H). LCMS: (ES) C₁₇H₂₁NO₄ requires 303, found 304 [M+H]⁺.

Step 6: Synthesis of 1-(Benzofuran-5-yl)-2-(methylamino) propan-1-one hydrochloride (Bk-5-MAPB HCl): To a stirred solution of tert-butyl (1-(benzofuran-5-yl)-1-oxopropan-2-yl)(methyl) carbamate (Boc-Bk-5-MAPB) (1.8 g, 5.94 mmol, 1 eq.) in dry DCM (15 ml) was added 4(M) HCl in 1,4 dioxane (15 ml) at 0° C. and the resulting reaction mixture was allowed to stir at room temperature for 3 hours. Upon completion of reaction (monitored by TLC, 10% EA in hexane), the solvents were evaporated, the crude was washed twice with diethyl ether (2×50 ml) and pentane, and them dried under vacuum to afford 1-(benzofuran-5-yl)-2-(methylamino)propan-1-one hydrochloride (Bk-5-MAPB HCl) (1.3 g, 91%) as off white solid. ¹HNMR(400 MHz, CDCl₃) δ 10.52 (bs, 1H), 9.28 (bs, 1H), 8.26 (bs, 1H), 7.93 (d, J=8.32 Hz, 1H), 7.71 (d, J=1.72 Hz, 1H), 7.58 (bd, J=9.12 Hz, 1H), 6.86 (bs, 1H), 5.08 (bs, 1H), 2.87 (s, 3H), 1.82 (q, 3H). LCMS: (ES) C₁₂H₁₃NO₂ requires 203, found 204 [M+H]⁺. HPLC: Purity (λ 220 nm): 98.40%.

### Synthesis 17. Synthesis of Bk-6-MAPB HCl

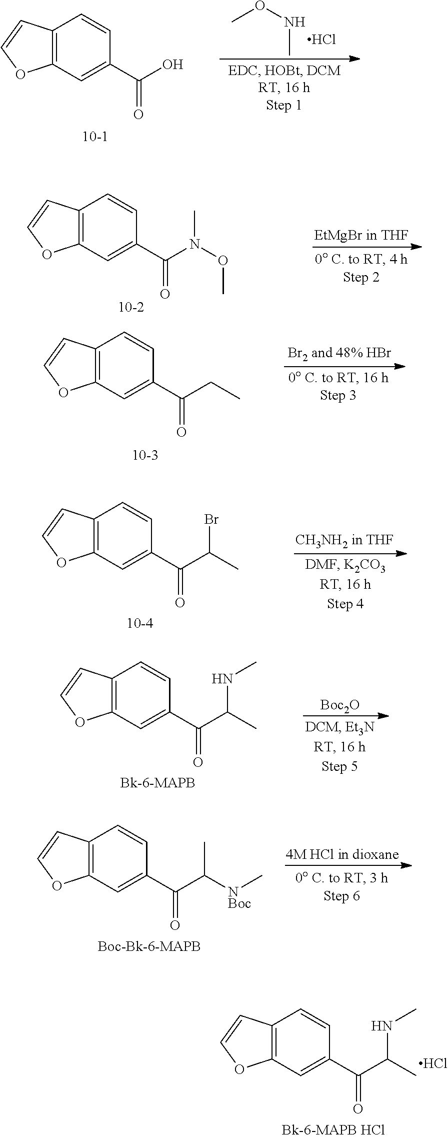

Step 1: Synthesis of N-methoxy-N-methylbenzofuran-6-carboxamide (10-2): To a stirred solution of benzofuran-6-carboxylic acid (10-1) (10 g, 61.72 mmol, 1 eq.) in dry DCM (100 ml) was added DIPEA (32 ml, 185.18 mmol, 3 eq.) followed by EDC.HCl (13 g, 67.90 mmol, 1.1 eq.) and HOBT (12.5 g, 92.59 mmol, 1.5 eq.) under N₂ atmosphere at room temperature and the resulting reaction mixture was allowed to stir at room temperature for 15 minutes. Then N, O-dimethylhydroxylamine hydrochloride (6.62 g, 67.90 mmol, 1.1 eq.) was added to the resulting reaction mixture and was allowed to stir at room temperature for 16 hours. Completion of the reaction was monitored by TLC (20% EA in hexane). Upon completion, the reaction mixture was extracted with DCM twice (2×200 ml) and washed with water followed by brine solution. The combined organic layers were dried over anhydrous sodium sulphate, solvent was removed under vacuum and purified by silica gel column chromatography using ethyl acetate/hexane (20:80 v/v) as eluent to afford pure N-methoxy-N-methylbenzofuran-6-carboxamide (10-2) as yellow sticky gum (11.4 g, 90%). ¹H NMR (400 MHz, CDCl₃) δ 7.88 (bs, 1H), 7.70 (d, J=2.08 Hz, 1H), 7.60 (s, 2H), 6.79 (d, J=1.16 Hz, 1H), 3.55 (s, 3H), 3.38 (s, 3H). LCMS: (ES) C₁₁H₁₁NO₃ requires 205, found 206 [M+H]⁺.

Step 2: Synthesis of 1-(benzofuran-6-yl) propan-1-one (10-3): To a stirred solution of N-methoxy-N-methylbenzofuran-6-carboxamide (10-2) (10 g, 48.73 mmol, 1 eq.) was added dry THE (150 ml) at 0° C. and followed by 3(M) solution of EtMgBr in diethyl ether (32.4 ml, 97.46 mmol, 2 eq.) to the reaction mixture and allowed to stir at room temperature for 4 hours. Upon completion, the reaction (monitored by TLC, 20% EA in hexane) was quenched with saturated NH₄Cl solution and extracted with ethyl acetate twice (2×100 ml), and washed with water and brine solution. The combined organic layers were dried over anhydrous sodium sulphate and solvent was evaporated under vacuum to afford crude compound 1-(benzofuran-6-yl) propan-1-one (10-3) as yellow solid (7 g, 82%). ¹H NMR (400 MHz, CDCl3) δ 8.12 (s, 1H), 7.90 (d, J=8.24 Hz, 1H), 7.76 (d, J=1.96 Hz, 1H), 7.65 (d, J=8.24 Hz, 1H), 6.81 (t, J=0.76 Hz & 0.92 Hz, 1H), 3.08 (q, 2H), 1.25 (t, J=7.28 Hz & 7.24 Hz, 3H). LCMS: (ES) C₁₁H₁₀O₂ requires 174, found 175 [M+H]⁺.

Step 3: Synthesis of 1-(benzofuran-6-yl)-2-bromopropan-1-one (10-4): To a stirred solution of 1-(benzofuran-6-yl)propan-1-one (10-3) (3 g, 17.22 mmol, 1 eq.) in dry THE (30 ml) was added hydrobromic acid 48% in water (30 ml, 551 mmol, 32 eq.) and bromine (0.97 ml, 18.94 mmol, 1.1 eq.) dropwise at 0° C. and the reaction mixture was allowed to stir at room temperature for 16 hours. Upon completion of the reaction (monitored by TLC, 10% EA in hexane), the reaction mixture was quenched with saturated sodium carbonate solution, extracted with ethyl acetate (2×100 ml), and washed with water and brine solution. The combined organic layers were dried over anhydrous sodium sulphate and solvent was evaporated under vacuum and purified by silica gel column chromatography using ethyl acetate/hexane (10:90 v/v) as eluent to afford pure compound 1-(benzofuran-6-yl)-2-bromopropan-1-one (10-4) as a yellow sticky gum (1.9 g, 43.6%). ¹H NMR (400 MHz, CDCl₃) δ 8.20 (bs, 1H), 7.94 (bd, J=8.16 Hz, 1H), 7.80 (d, J=2 Hz, 1H), 7.68 (bd, J=8.2 Hz, 1H), 6.83 (bs, 1H), 5.37 (q, 1H), 1.93 (d, J=6.68 Hz, 3H). LCMS: (ES) C₁₁H₁₉BrO₂ requires 252, found 253 [M+H]⁺.

Step 4: Synthesis of 1-(benzofuran-6-yl)-2-(methylamino) propan-1-one (Bk-6-MAPB): To a stirred solution of 1-(benzofuran-6-yl)-2-bromopropan-1-one (16-4) (3.8 g, 15 mmol, 1eq.) in dry DMF (30 ml) was added potassium carbonate (3.1 g, 22.53 mmol, 1.5 eq.) and methyl amine 2(M) in THE (45 ml, 90.11 mmol, 6 eq.) in a sealed round bottom flask and the resulting reaction mixture was allowed to stir at room temperature for 16 hours. Upon completion of the reaction (monitored by TLC, 10% EA in hexane), the crude was extracted with ethyl acetate (2×50 ml) and washed with water (2×50 ml) and brine solution. The combined organic solvent was dried over anhydrous sodium sulphate and solvent was evaporated under vacuum to afford crude 1-(benzofuran-6-yl)-2-(methylamino) propan-1-one (Bk-6-MAPB) as a yellow sticky gum (3 g, 98%). ¹H NMR (400 MHz, CDCl₃) δ 8.14 (s, 1H), 7.90 (d, J=8.2 Hz, 1H), 7.78 (d, J=1.96 Hz, 1H), 7.68 (d, J=8.2 Hz, 1H), 6.83 (s, 1H), 4.29 (q, 1H), 2.38 (s, 3H), 1.34 (d, J=6.96 Hz, 3H). LCMS: (ES) C₁₂H₁₃NO₂ requires 203, found 204 [M+H]⁺.

Step 5: Synthesis of tert-butyl (1-(benzofuran-6-yl)-1-oxopropan-2-yl) (methyl) carbamate (Boc-Bk-6-MAPB): To a stirred solution of 1-(benzofuran-6-yl)-2-(methylamino) propan-1-one (Bk-6-MAPB) (3 g, 14.77 mmol, 1eq.) in dry DCM (30 ml) was added triethylamine (4.26 ml, 29.55 mmol, 2 eq.) and Boc anhydride (6.78 ml, 29.55 mmol, 2 eq.) and the resulting reaction mixture was allowed to stir at room temperature for 4 hours. Upon completion of the reaction (monitored by TLC, 10% EA in hexane), the reaction mixture was extracted with DCM (2×50 ml) and washed with water followed by brine solution. The combined organic solvent was dried over anhydrous sodium sulphate and the solvent was evaporated under vacuum and purified by silica gel column chromatography using ethyl acetate/hexane (10:90 v/v) as eluent to afford tert-butyl (1-(benzofuran-6-yl)-1-oxopropan-2-yl)(methyl) carbamate (Boc-Bk-6-MAPB) as a yellow sticky gum (2.5 g, 55%). 1H NMR(400 MHz, CDCl₃) δ 8.20-8.11 (bs, 1H), 7.93-7.85 (bd, 1H), 7.76 (s, 1H), 7.63 (bs, 1H), 6.80 (s, 1H), 5.77-5.31 (m, 1H), 2.76-2.58 (s, 3H), 1.45 (s, 9H), 1.38 (m, 3H). Rotamers observed. LCMS: (ES) C₁₇H₂₁NO₄ requires 303, found 304 [M+H]⁺.

Step 6: Synthesis of 1-(benzofuran-6-yl)-2-(methylamino) propan-1-one hydrochloride (Bk-6-MAPB HCl): To a stirred solution of tert-butyl (1-(benzofuran-6-yl)-1-oxopropan-2-yl)(methyl) carbamate (Boc-Bk-6-MAPB) (1.5 g, 4.95 mmol, 1 eq.) in dry DCM (15 ml) was added 4(M) HCl in 1,4 dioxane (15 ml) at 0° C. and the resulting reaction mixture was allowed to stir at room temperature for 3 hours. Upon completion of reaction (monitored by TLC, 10% EA in hexane), the solvent was evaporated, and the crude was washed twice with diethyl ether (2×50 ml) and pentane and dried under vacuum to afford 1-(benzofuran-6-yl)-2-(methylamino)propan-1-one hydrochloride (HCl Bk-6-MAPB) (1.1 g, 92%) as off white solid. ¹HNMR (400 MHz, CDCl₃) δ 10.90 (s, 1H), 8.92 (s, 1H), 8.13 (s, 1H), 7.84 (bd, J=6.88 Hz, 1H), 7.72 (bd, J=8.16 Hz, 1H), 6.86 (s, 1H), 4.96 (bs, 1H), 2.86 (s, 3H), 1.85 (d, J=7.08 Hz, 3H). LCMS: (ES) C12H13NO2 requires 203, found 204 [M+H]⁺. HPLC: Purity (λ 220 nm): 99.85%.

### Synthesis 18. Synthesis of Bk-5-MBPB HCl

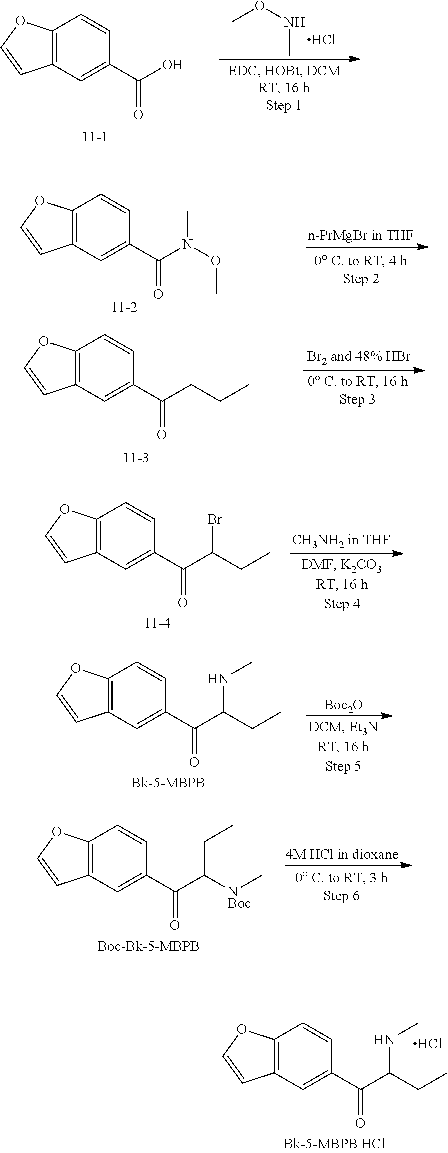

Step 1: Synthesis of N-methoxy-N-methylbenzofuran-5-carboxamide (11-2): To a stirred solution of benzofuran-5-carboxylic acid (11-1) (10 g, 61.72 mmol, 1 eq.) in dry DCM (100 ml) was added DIPEA (32 ml, 185.18 mmol, 3 eq.) followed by EDC.HCl (13 g, 67.90 mmol, 1.1 eq.) and HOBT (12.5 g, 92.59 mmol, 1.5 eq.) under N₂ atmosphere at room temperature and the resulting reaction mixture was allowed to stir at room temperature for 15 minutes. Then N, O-dimethylhydroxylamine hydrochloride (6.62 g, 67.90 mmol, 1.1 eq.) was added to the resulting reaction mixture and was allowed to stir at room temperature for 16 hours. Upon completion, monitored by TLC (20% EA in hexane), the reaction mixture was extracted with DCM twice (2×200 ml) and washed with water followed by brine solution. The combined organic layers were dried over anhydrous sodium sulphate and solvent was removed under vacuum and purified by silica gel column chromatography using ethyl acetate/hexane (20:80 v/v) as eluent to afford pure N-methoxy-N-methylbenzofuran-5-carboxamide (11-2) as yellow sticky gum (10.6 g, 83%). ¹H NMR (400 MHz, CDCl3) δ 7.97 (s, 1H), 7.66 (m, 2H), 7.50 (d, J=8.56 Hz, 1H), 6.80 (d, J=1.08 Hz, 1H), 3.54 (s, 3H), 3.37 (s, 3H). LCMS: (ES) C₁₁H₁₁NO₃ requires 205, found 206 [M+H]⁺.

Step 2: Synthesis of 1-(benzofuran-5-yl) butan-1-one (11-3): To a stirred solution of N-methoxy-N-methylbenzofuran-5-carboxamide (11-2) (5 g, 24.37 mmol, 1 eq.) was added in dry THE (50 ml) at 0° C. and was added 2 (M) solution of n-propylMgBr in THE (24.4 ml, 48.73 mmol, 2 eq.) to the reaction mixture and allowed to stir at room temperature for 4 hours. Upon completion, (monitored by TLC, 20% EA in hexane) the reaction was quenched with saturated NH₄Cl solution and extracted with ethyl acetate twice (2×75 ml) and then washed with water followed by brine solution. The combined organic layers were dried over anhydrous sodium sulphate, solvent was evaporated under vacuum to afford crude compound 1-(benzofuran-5-yl) butan-1-one (11-3) as yellow solid (4.5 g, 98%). ¹H NMR (400 MHz, CDCl3) δ 8.25 (d, J=1.56 Hz, 1H), 7.97 (dd, J=1.72 Hz, 8.72 Hz, 1H), 7.67 (d, J=2.2 Hz, 1H), 7.53 (d, J=8.72 Hz, 1H), 6.84 (d, J=1.88 Hz, 1H), 2.99 (t, J=7.28 Hz, 7.36 Hz, 2H), 1.83 (q, 2H), 1.01 (t, J=7.4 Hz, 3H). LCMS: (ES) C₁₂H₁₂O₂ requires 188, found 189 [M+H]⁺.

Step 3: Synthesis of 1-(benzofuran-5-yl)-2-bromobutan-1-one (11-4): To a stirred solution of 1-(benzofuran-5-yl)butan-1-one (11-3) (3 g, 15.95 mmol, 1 eq.) in dry THE (30 ml) was added hydrobromic acid 48% in water (41.3 ml, 510.63 mmol, 32 eq.) and bromine (0.89 ml, 17.55 mmol, 1.1 eq.) dropwise at 0° C. and the reaction mixture was allowed to stir at room temperature for 16 hours. Upon completion, (monitored by TLC, 10% EA in hexane), the reaction mixture was quenched with saturated sodium carbonate solution, extracted with ethyl acetate (2×50 ml), and washed with water and brine solution. The combined organic layers were dried over anhydrous sodium sulphate and solvent was evaporated under vacuum and purified by silica gel column chromatography using ethyl acetate/hexane (10:90 v/v) as eluent to afford pure compound 1-(benzofuran-5-yl)-2-bromobutan-1-one (11-4) as yellow sticky gum (3.2 g, 75%). ¹H NMR (400 MHz, CDCl₃) δ 8.32 (d, J=1.32 Hz, 1H), 8.02 (dd, J=1.52 Hz, 8.72 Hz, 1H), 7.70 (d, J=2.08 Hz, 1H), 7.57 (d, J=8.72 Hz, 1H), 6.87 (d, J=1.8 Hz, 1H), 5.14 (t, J=7.04 Hz, 7.08 Hz, 1H), 2.30 (m, 2H), 1.09 (t, J=7.64 Hz, 7.28 Hz, 3H). LCMS: (ES) C₁₂H₁₁BrO₂ requires 267, found 268 [M+H]⁺

Step 4: Synthesis of 1-(benzofuran-5-yl)-2-(methylamino) butan-1-one (11-5): To a stirred solution of 1-(benzofuran-5-yl)-2-bromobutan-1-one (11-4) (3.2 g, 11.98 mmol, 1eq.) in dry DMF (30 ml) was added potassium carbonate (2.48 g, 17.97 mmol, 1.5 eq.) and methyl amine 2(M) in THF (36 ml, 71.91 mmol, 6 eq.) in a sealed round bottom flask and the resulting reaction mixture was allowed to stir at room temperature for 16 hours. Upon completion of the reaction (monitored by TLC, 10% EA in hexane), volatiles were evaporated, and the crude was extracted with ethyl acetate (2×50 ml) and washed with water (2×50 ml) and brine solution. The combined organic solvent was dried over anhydrous sodium sulphate and solvent was evaporated under vacuum to afford crude 1-(benzofuran-5-yl)-2-(methylamino) butan-1-one (Bk-5-MBPB) as yellow sticky gum (2.3 g, 88%). ¹H NMR (400 MHz, CDCl₃) δ 8.26 (d, J=1.12 Hz, 1H), 7.98 (dd, J=1.40 Hz, 8.64 Hz, 1H), 7.69 (d, J=1.96 Hz, 1H), 7.57 (d, J=8.6 Hz, 1H), 6.86 (d, J=1.16 Hz, 1H), 4.15 (t, J=5.76 Hz, 5.80 Hz, 1H), 2.37 (s, 3H), 1.86 (m, 1H), 1.63 (m, 1H), 0.92 (t, J=7.44 Hz, 3H). LCMS: (ES) C₁₃H₁₅NO₂ requires 217, found 218 [M+H]⁺.

Step 5: Synthesis of tert-butyl (1-(benzofuran-5-yl)-1-oxobutan-2-yl) (methyl) carbamate (Boc-Bk-5-MBPB): To a stirred solution of 1-(benzofuran-5-yl)-2-(methylamino) butan-1-one (Bk-5-MBPB) (2.3 g, 10.59 mmol, 1 eq.) in dry DCM (30 ml) was added triethylamine (3.05 ml, 21.19 mmol, 2 eq.) and Boc anhydride (4.86 ml, 21.19 mmol, 2 eq.) and the resulting reaction mixture was allowed to stir at room temperature for 4 hours. Upon completion, (monitored by TLC, 10% EA in hexane), the reaction mixture was extracted with DCM (2×50 ml) and washed with water followed by brine solution. Combined organic solvent was dried over anhydrous sodium sulphate, solvent was evaporated under vacuum and purified by silica gel column chromatography using ethyl acetate/hexane (10:90 v/v) as eluent to afford pure tert-butyl (1-(benzofuran-5-yl)-1-oxobutan-2-yl)(methyl)carbamate (Boc-Bk-5-MBPB) as a yellow sticky gum (1.7 g, 50%). ¹H NMR (400 MHz, CDCl₃) δ 8.38 (s, 1H), 8.03 (dd, J=8.76 Hz, 1H), 7.68 (m, 1H), 7.52 (d, J=4.8 Hz, 1H), 6.82 (s, 1H), 5.62 (m, 1H), 2.67 (s, 3H), 1.97 (m, 1H), 1.78 (m, 1H), 1.52 (s, 9H), 0.96 (m, 3H). Rotamer observed. LCMS: (ES) C₁₈H₂₃NO₄ requires 317, found 318 [M+H]⁺.

Step 6: Synthesis of 1-(benzofuran-5-yl)-2-(methylamino)butan-1-one hydrochloride (Bk-5-MBPB HCl): To a stirred solution of tert-butyl (1-(benzofuran-5-yl)-1-oxobutan-2-yl)(methyl) carbamate (Boc-Bk-5-MBPB) (1.5 g, 4.73 mmol, 1 eq.) in dry DCM (15 ml) was added 4(M) HCl in 1,4 dioxane (15 ml) at 0° C. and the resulting reaction mixture was allowed to stir at room temperature for 3 hours. Upon completion of reaction (monitored by TLC, 10% EA in hexane), the solvent was evaporated, and the crude was washed twice with diethyl ether (2×30 ml) and pentane and dried under vacuum to afford 1-(benzofuran-5-yl)-2-(methylamino)butan-1-one hydrochloride (HCl Bk-5-MBPB) (1.15 g, 95%) as off white solid. ¹H NMR (400 MHz, CDCl₃) δ 10.51 (s, 1H), 9.10 (s, 1H), 8.31 (s, 1H), 7.97 (d, J=8.32 Hz, 1H), 7.72 (s, 1H), 7.60 (d, J=8.32 Hz, 1H), 6.88 (s, 1H), 5.12 (s, 1H), 2.86 (s, 3H), 2.41 (bs, 1H), 2.22 (bs, 1H), 1.87 (s, 2H), 1.03 (t, J=6.28 Hz, 6.48 Hz, 3H). LCMS: (ES) C₁₃H₁₅NO₂ requires 217, found 218 [M+H]⁺. HPLC: Purity (λ 220 nm): 96.94%.

### Synthesis 19. Synthesis of Bk-6-MBPB HCl

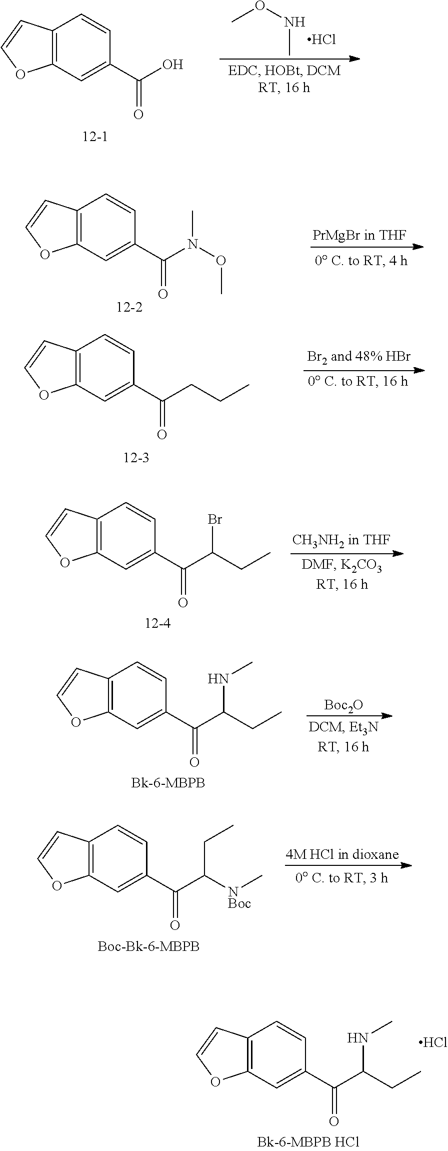

Step 1: Synthesis of N-methoxy-N-methylbenzofuran-6-carboxamide (12-2): To a stirred solution of benzofuran-6-carboxylic acid (12-1) (10 g, 61.72 mmol, 1 eq.) in dry DCM (100 mL) was added DIPEA (32 ml, 185.18 mmol, 3 eq.) followed by EDC.HCl (13 g, 67.90 mmol, 1.1 eq.) and HOBT (12.5 g, 92.59 mmol, 1.5 eq.) under N₂ atmosphere at room temperature and the resulting reaction mixture was allowed to stir at room temperature for 15 minutes. Then N, O-dimethylhydroxylamine hydrochloride (6.62 g, 67.90 mmol, 1.1 eq.) was added to the resulting reaction mixture and was allowed to stir at room temperature for 16 hours. Upon completion (monitored by TLC 20% EA in hexane), the reaction mixture was extracted with DCM twice (2×200 ml) and washed with water followed by brine solution. The combined organic layers were dried over anhydrous sodium sulphate and solvent was removed under vacuum and purified by silica gel column chromatography using ethyl acetate/hexane (20:80 v/v) as eluent to afford pure N-methoxy-N-methylbenzofuran-6-carboxamide (12-2) as yellow sticky gum (10.6 g, 83%). ¹H NMR (400 MHz, CDCl₃) δ 7.97 (bs, 1H), 7.66 (m, 2H), 7.50 (d, J=8.56 Hz, 1H), 6.80 (s, 1H), 3.54 (s, 3H), 3.37 (s, 3H). LCMS: (ES) C₁₁H₁₁NO₃ requires 205, found 206 [M+H]⁺.

Step 2: Synthesis of 1-(benzofuran-6-yl)butan-1-one (12-3): To a stirred solution of N-methoxy-N-methylbenzofuran-6-carboxamide (12-2) (10 g, 48.73 mmol, 1 eq.) was added dry THE (100 mL) at 0° C. and 2 (M) solution of n-propylmagnesium bromide in THE (48.73 mL, 97.46 mmol, 2 eq.). The reaction mixture and allowed to stir at room temperature for 4 hours. Upon completion of reaction (monitored by TLC, 20% EA in hexane) was quenched with saturated NH₄Cl solution and extracted with ethyl acetate twice (2×200 ml), and then washed with water followed by brine solution. The combined organic layers were dried over anhydrous sodium sulphate and solvent was evaporated under vacuum to afford crude 1-(benzofuran-6-yl)butan-1-one (12-3) as yellow solid (9 g, 98%). ¹H NMR (400 MHz, CDCl3) δ 8.11 (s, 1H), 7.89 (d, J=8.2 Hz, 1H), 7.76 (d, J=2.04 Hz, 1H), 7.64 (d, J=8.16 Hz, 1H), 6.81 (d, J=1.3 Hz, 1H), 3.04 (m, 2H), 1.84 (m, 2H), 1.03 (t, J=7.4 Hz, 3H). LCMS: (ES) C₁₂H₁₂O₂ requires 188, found 189 [M+H]⁺.

Step 3: Synthesis of 1-(benzofuran-6-yl)-2-bromobutan-1-one (12-4): To a stirred solution of 1-(benzofuran-6-yl)butan-1-one (12-3) (4.6 g, 24.46 mmol, 1 eq.) in dry THE (50 mL) was added hydrobromic acid 48% in water (42.51 ml, 782.97 mmol, 32 eq.) and bromine (1.37 mL, 26.91 mmol, 1.1 eq.) dropwise at 0° C. and the reaction mixture was allowed to stir at room temperature for 16 hours. Upon completion, the reaction mixture (monitored by TLC, 10% EA in hexane) was quenched with saturated sodium carbonate solution, extracted with ethyl acetate (2×100 ml), and washed with water and brine solution. The combined organic layers were dried over anhydrous sodium sulphate, solvent was evaporated under vacuum and purified by silica gel column chromatography using ethyl acetate/hexane (10:90 v/v) as eluent to afford pure 1-(benzofuran-6-yl)-2-bromobutan-1-one (12-4) as yellow sticky gum (3.8 g, 58%). ¹H NMR (400 MHz, CDCl₃) δ 8.18 (s, 1H), 7.93 (d, J=7.16 Hz, 1H), 7.80 (d, J=2.08 Hz, 1H), 7.68 (d, J=8.04 Hz, 1H), 6.83 (s, 1H), 5.12 (t, J=7.12 Hz, 6.72 Hz, 1H), 2.28 (m, 2H), 1.09 (t, J=7.28 Hz, 7.32 Hz, 3H). LCMS: (ES) C₁₂H₁₁BrO₂ requires 267, found 268 [M+H]⁺.

Step 4: Synthesis of 1-(benzofuran-6-yl)-2-(methylamino)butan-1-one (Bk-6-MBPB): To a stirred solution of 1-(benzofuran-6-yl)-2-bromobutan-1-one (12-4) (3.8 g, 14.22 mmol, 1 eq.) in dry DMF (40 mL) was added potassium carbonate (2.94 g, 21.33 mmol, 1.5 eq.) and methyl amine 2(M) in THE (42.5 mL, 85.37 mmol, 6 eq.) in a sealed round bottom flask and the resulting reaction mixture was allowed to stir at room temperature for 16h. Upon completion of reaction (monitored by TLC, 10% EA in Hexane), volatiles were evaporated, and the crude was extracted with ethyl acetate (2×100 ml), washed with water (2×50 ml) and brine solution. Combined organic solvent was dried over anhydrous sodium sulphate, solvent was evaporated under vacuum to afford crude 1-(benzofuran-6-yl)-2-(methylamino)butan-1-one (Bk-6-MBPB) as yellow sticky gum (2.75 g, 89%). Crude ¹H NMR (400 MHz, CDCl₃) δ 8.14 (s, 1H), 7.90 (d, J=0.96 Hz, 8.0 Hz, 1H), 7.79 (d, J=2.04 Hz, 1H), 7.68 (d, J=8.2 Hz, 1H), 6.83 (d, J=1 Hz, 1H), 4.14 (t, J=6.36 Hz, 5.48 Hz, 1H), 2.37 (s, 3H), 1.86 (m, 1H), 1.60 (m, 1H), 0.92 (t, J=7.44 Hz, 3H). LCMS: (ES) C₁₃H₁₅NO₂ requires 217, found 218 [M+H]⁺.

Step 5: Synthesis of tert-butyl (1-(benzofuran-6-yl)-1-oxobutan-2-yl)(methyl)carbamate (Boc-Bk-6-MBPB): To a stirred solution of 1-(benzofuran-6-yl)-2-(methylamino)butan-1-one (Bk-6-MBPB) (2.75 g, 12.65 mmol, 1eq.) in dry DCM (30 mL) was added triethylamine (3.65 mL, 25.31 mmol, 2 eq.) and Boc anhydride (5.8 mL, 25.31 mmol, 2 eq.) and the resulting reaction mixture was allowed to stir at room temperature for 4 hours. Upon completion of the reaction (monitored by TLC, 10% EA in hexane), the reaction mixture was extracted with DCM (2×50 ml) and washed with water followed by brine solution. The combined organic layers were dried over anhydrous sodium sulphate, solvent was evaporated under vacuum, and the crude material purified by silica gel column chromatography using ethyl acetate/hexane (10:90 v/v) as eluent to afford pure tert-butyl (1-(benzofuran-6-yl)-1-oxobutan-2-yl)(methyl)carbamate (Boc-Bk-6-MBPB) as yellow sticky gum (3.4 g, 84%). ¹H NMR (400 MHz, CDCl₃) δ 8.24 (s, 1H), 7.97 (dd, J=8.2 Hz, 1H), 7.76 (bs, 1H), 7.63 (bm, 1H), 6.80 (bs, 1H), 5.61 (t, J=5.64 Hz, 8.88 Hz, 1H), 2.66 (s, 3H), 1.99 (q, 2H), 1.55 (s, 9H), 0.98 (m, 3H). Rotamer observed. LCMS: (ES) C₁₈H₂₃NO₄ requires 317, found 318 [M+H]⁺.

Step 6: Synthesis of 1-(benzofuran-6-yl)-2-(methylamino)butan-1-one hydrochloride Bk-6-MBPB HCl): To a stirred solution of tert-butyl (1-(benzofuran-6-yl)-1-oxobutan-2-yl)(methyl)carbamate (Boc-Bk-6-MBPB) (1.5 g, 4.73 mmol, 1 eq.) in dry DCM (15 mL) was added 4(M) HCl in 1,4 dioxane (15 mL) at 0° C. and the resulting reaction mixture was allowed to stir at room temperature for 3 hours. Upon completion of the reaction (monitored by TLC, 10% EA in hexane), the solvent were evaporated and the crude was washed twice with diethyl ether (2×50 ml) and pentane and dried under vacuum to afford 1-(benzofuran-6-yl)-2-(methylamino)butan-1-one hydrochloride (Bk-6-MBPB HCl) (1 g, 83%) as a white solid. ¹H NMR (400 MHz, CDCl₃) δ 10.78 (s, 1H), 8.95 (s, 1H), 8.15 (s, 1H), 7.87 (m, 2H), 7.72 (d, J=8.08 Hz, 1H), 6.86 (d, J=1.88 Hz, 1H), 4.99 (bs, 1H), 2.86 (bs, 3H), 2.48 (m, 1H), 2.71 (m, 1H), 1.05 (m, 3H). LCMS: (ES) C₁₃H₁₅NO₂ requires 217, found 218 [M+H]⁺. HPLC: Purity (λ 300 nm): 99.68%.

### Synthesis 20. Synthesis of (R)-1-(benzofuran-5-yl)-N-methylpropan-2-amine (R-5-MAPB)

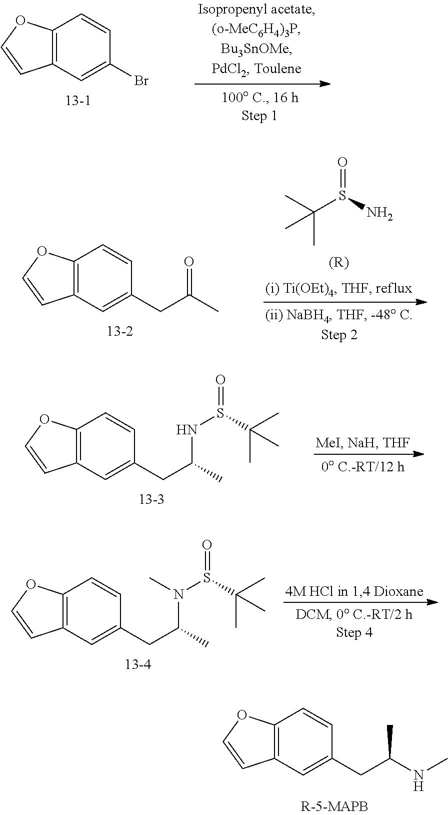

Step 1: To a stirred solution of 5-bromobenzofuran (13-1) (20 g, 101.52 mmol, 1 eq.) in dry Toluene (400 ml) was added tri(o-tolyl)phosphine (1.84 g, 6.091 mmol, 0.06 eq.), tributyl tin methoxide (48.89 mL, 152.28 mmol, 1.5 eq.) and Isopropenyl acetate (16.99 mL, 156.34 mmol, 1.54 eq.) and the resulting reaction mixture was degassed under nitrogen for 15 minutes. Then palladium (II) chloride (1.26 g, 7.10 mmol, 0.07 eq.) was added to the reaction mixture and the resulting reaction mixture was heated to 100° C. for 16 hrs. Upon completion, monitored by TLC (10% EA in Hexane), the reaction mixture was filtered through celite bed, extracted with ethyl acetate (2×400 ml), washed with water, followed by saturated potassium fluoride solution, and brine solution. Combined organic layer was dried over anhydrous sodium sulphate, solvent was removed under vacuum and purified by silica gel column chromatography using ethyl acetate/hexane (10:90 v/v) as eluent to afford 1-(benzofuran-5-yl)propan-2-one (13-2) as light yellow gum (17 g, 96%). ¹H NMR (400 MHz, DMSO-d₆) δ 7.96 (d, J=2.08 Hz, 1H), 7.53 (d, J=8.48 Hz, 1H), 7.46 (s, 1H), 7.13 (dd, J=1.52 Hz, 8.44 Hz, 1H), 6.92 (d, J=0.76 Hz, 1H), 3.83 (s, 2H), 2.12 (s, 3H). LCMS: (ES) C₁₁H₁₀O₂ requires 174, found 175 [M+H]⁺.

Step 2: To a stirred solution of 1-(benzofuran-5-yl)propan-2-one (13-2) (9 g, 51.66 mmol, 1eq.) in dry THF (150 ml) was added Ti(OEt)₄ (37.91 ml, 180.82 mmol, 3.5eq.) and (R)-2-methylpropane-2-sulfinamide (6.26 g, 51.66 mmol, 1eq.) (dissolved in 30 ml dry THF) and the resulting reaction mixture was allowed to stir at 70° C. for 12 hrs. Upon completion, monitored by TLC (50% EA in Hexane), the reaction mixture was cooled to 0° C., gradually to −48° C. and NaBH₄ (7.81 g, 206.65 mmol, 4 eq.) (dissolved in 30 ml dry THF) was added into the reaction mixture at −48° C. and the resulting reaction mixture was allowed to stir at −48° C. for 3 hrs. Upon completion, monitored by TLC (50% EA in Hexane), the reaction mixture was taken to room temperature and was quenched with Methanol and Sat NaCl solution (until white precipitate observed). The reaction mixture was then filtered through celite bed, washed with methanol (2×150 ml) and ethyl acetate (2×150 ml), evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (R)-N-((R)-1-(benzofuran-5-yl)propan-2-yl)-2-methylpropane-2-sulfinamide (13-3) as yellow sticky gum (14 g, 96%). ¹H NMR (400 MHz, DMSO-d₆) δ 7.94 (s, 1H), 7.48 (m, 2H), 7.15 (d, J=8.32 Hz, 1H), 6.89 (d, J=7.76 Hz, 1H), 4.97 (d, J=6.04 Hz, 1H), 3.48 (m, 1H), 3.07 (m, 1H), 2.76 (m, 1H), 1.09 (s, 12H), 1.08 (m, 3H) LCMS: (ES) C₁₅H₂₁NO2S requires 279, found 280 [M+H]⁺.

Step 3: To a stirred solution of (R)-N-((R)-1-(benzofuran-5-yl)propan-2-yl)-2-methylpropane-2-sulfinamide (13-3) (15 g, 53.57 mmol, 1 eq.) in dry THE (100 mL) (In a sealed tube) was added NaH (60%) (4.28 g, 107.14 mmol, 2 eq.) at 0° C. and the resulting reaction mixture was allowed to stir at 0° C. for 30 min. Then Iodomethane (6.7 ml, 107.14 mmol, 2 eq.) was added at 0° C. and the resulting reaction mixture was allowed to stir at room temperature for 12h. Upon completion, monitored by TLC (50% EA in Hexane), the reaction mixture was quenched with ice water, extracted with ethyl acetate (2×250 ml), washed with saturated ammonium chloride solution, followed by brine solution. Combined organic layer was dried over anhydrous sodium sulphate, solvent was removed under vacuum and purified by silica gel column chromatography using ethyl acetate/hexane (50:50 v/v) as eluent to afford (R)-N-((R)-1-(benzofuran-5-yl)propan-2-yl)-N,2-dimethylpropane-2-sulfinamide (13-4) as light yellow gum (8 g, 50.9%). ¹H NMR (400 MHz, DMSO-d₆) δ 7.93 (s, 1H), 7.49 (m, 2H), 7.14 (d, J=7.4, 1H), 6.89 (s, 1H), 3.54 (m, 1H), 2.92 (m, 1H), 2.81 (m, 1H), 2.49 (s, 3H), 1.09 (d, J=6.64 Hz, 3H), 1.02 (s, 9H). LCMS: (ES) C₁₆H₂₃NO₂S requires 293, found 294 [M+H]⁺.

Step 4: To a stirred solution of (R)-N-((R)-1-(benzofuran-5-yl)propan-2-yl)-N,2-dimethylpropane-2-sulfinamide (13-4) (10.5 g, 37.58 mmol, 1 eq.) in dry DCM (50 ml) was added 4M HCl in 1,4 dioxane (100 mL) at 0° C. and then the resulting reaction mixture was allowed to stir at room temperature for 2h. Upon completion of reaction (monitored by TLC, 30% EA in Hexane), the solvent were evaporated and the crude was washed twice with diethyl ether (2×60 ml) and pentane and dried under vacuum to afford (R)-1-(benzofuran-5-yl)-N-methylpropan-2-amine hydrochloride (R-5-MAPB) (5.8 g, 81%) as off white solid. ¹HNMR(400 MHz, DMSO-d₆) δ 9.00 (bs, 2H), 7.99 (d, J=1.6 Hz, 1H), 7.57 (m, 2H), 7.21 (d, J=7.8 Hz, 1H), 6.93 (s, 1H), 3.38 (bs, 1H), 3.25 (m, 1H), 2.77 (m, 1H), 2.56 (s, 3H), 1.11 (d, J=6.28 Hz, 3H). LCMS: (ES) C₁₂H₁₅NO requires 189, found 190 [M+H]⁺. HPLC: Purity (λ 210 nm): 99.26%.

### Synthesis 21. Synthesis of (S)-1-(benzofuran-5-yl)-N-methylpropan-2-amine (S-5-MAPB)

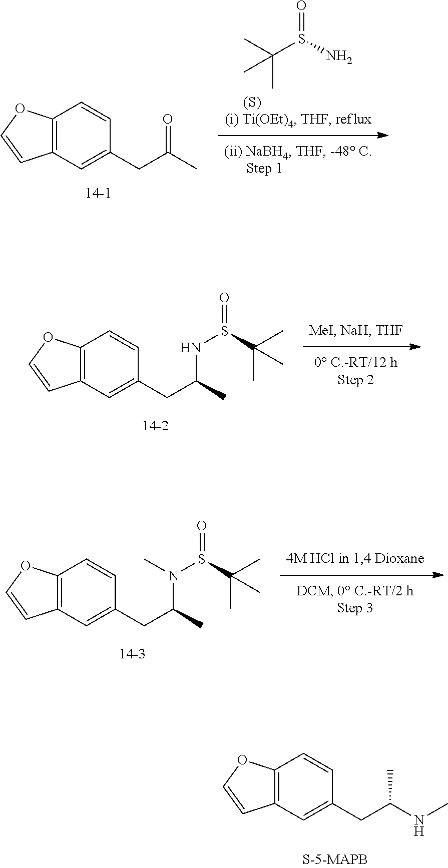

Step 1: To a stirred solution of 1-(benzofuran-5-yl)propan-2-one (14-1) (5 g, 28.70 mmol, eq.) in dry THE (100 ml) was added Ti(OEt)₄ (21.06 ml, 100.45 mmol, 3.5 eq.) and (S)-2-methylpropane-2-sulfinamide (3.47 g, 28.73 mmol, 1eq.) (dissolved in 20 ml dry THF) and the resulting reaction mixture was allowed to stir at 70° C. for 12 hrs. Upon completion (monitored by TLC, 50% EA in Hexane), the reaction mixture was cooled to 0° C., gradually to −48° C. and NaBH₄ (4.34 g, 114.81 mmol, 4 eq.) (dissolved in 20 ml dry THF) was added into the reaction mixture at −48° C. and the resulting reaction mixture was allowed to stir at −48° C. for 3 hrs. Upon completion (monitored by TLC, 50% EA in Hexane), the reaction mixture was taken to room temperature and was quenched with Methanol and Sat. NaCl solution (until white precipitate observed). The reaction mixture was then filtered through celite bed, washed with methanol (2×100 ml) and ethyl acetate (2×100 ml), evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (S)-N-((S)-1-(benzofuran-5-yl)propan-2-yl)-2-methylpropane-2-sulfinamide (14-2) as yellow sticky gum (6.5 g, 81%). Crude 1H NMR (400 MHz, DMSO-d₆) δ 7.94 (d, J=7.8 Hz, 1H), 7.50 (m, 2H), 7.14 (m, 1H), 6.90 (d, J=6.36 Hz, 1H), 6.90 (d, J=6.36 Hz, 1H), 4.97 (d, J=5.96 Hz, 1H), 3.48 (m, 1H), 3.08 (m, 1H), 2.76 (m, 1H), 1.18 (m, 12H). LCMS: (ES) C₁₅H₂₁NO₂S requires 279, found 280 [M+H]⁺.

Step 2: To a stirred solution of (S)-N-((S)-1-(benzofuran-5-yl)propan-2-yl)-2-methylpropane-2-sulfinamide (14-2) (7 g, 25 mmol, 1 eq.) in dry THE (50 mL) (In a sealed tube) was added NaH (60%) (2 g, 50 mmol, 2 eq.) at 0° C. and the resulting reaction mixture was allowed to stir at 0° C. for 30 min. Then Iodomethane (3.11 ml, 50 mmol, 2 eq.) was added at 0° C. and the resulting reaction mixture was allowed to stir at room temperature for 12h. Upon completion (monitored by TLC, 50% EA in Hexane), the reaction mixture was quenched with ice water, extracted with ethyl acetate (2×200 ml), washed with saturated ammonium chloride solution, followed by brine solution. Combined organic layer was dried over anhydrous sodium sulphate, solvent was removed under vacuum and purified by silica gel column chromatography using ethyl acetate/hexane (50:50 v/v) as eluent to afford (S)-N-((S)-1-(benzofuran-5-yl)propan-2-yl)-N,2-dimethylpropane-2-sulfinamide (14-3) as light yellow gum (4 g, 54%). 1H NMR(400 MHz, DMSO-d₆) δ 7.94 (s, 1H), 7.49 (t, J=8.4 Hz, 9.04 Hz, 2H), 7.14 (d, J=8.2, 1H), 6.89 (s, 1H), 3.55 (m, 1H), 2.92 (m, 1H), 2.88 (m, 1H), 2.51 (s, 3H), 1.27 (m, 3H), 1.07 (S, 9H). LCMS: (ES) C₁₆H₂₃NO₂S requires 293, found 294 [M+H]⁺.

Step 3: To a stirred solution of (S)-N-((S)-1-(benzofuran-5-yl)propan-2-yl)-N,2-dimethylpropane-2-sulfinamide (14-3) (7 g, 23.89 mmol, 1 eq.) in dry DCM (35 mL) was added 4M-HCl in 1,4 dioxane (70 mL) at 0° C. and the resulting reaction mixture was allowed to stir at room temperature for 2h. Upon completion of reaction (monitored by TLC, 30% EA in Hexane), the solvent was evaporated, and the crude was washed twice with diethyl ether (2×60 ml) and pentane and dried under vacuum to afford (S)-1-(benzofuran-5-yl)-N-methylpropan-2-amine hydrochloride (S-5-MAPB) (5 g, 97%) as off white solid. ¹HNMR(400 MHz, DMSO-d₆) δ 9.06 (bs, 2H), 7.99 (d, J=1.88 Hz, 1H), 7.57 (m, 2H), 7.21 (d, J=8.28 Hz, 1H), 6.93 (d, J=1.32 Hz, 1H), 3.33 (m, 1H), 3.26 (m, 1H), 2.77 (q, 1H), 2.56 (s, 3H), 1.11 (d, J=6.4 Hz, 3H), LCMS: (ES) C₁₂H₁₅NO requires 189, found 190 [M+H]⁺. HPLC: Purity (λ 250 nm): 99.81%.

### Synthesis 22. Synthesis of (R)-1-(benzofuran-6-yl)-N-methylpropan-2-amine (R-6-MAPB)

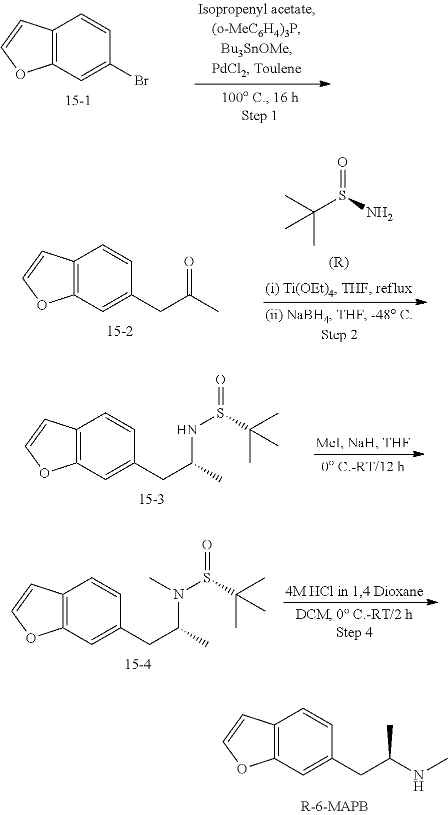

Step 1: A mixture of 6-bromobenzofuran (15-1) (10 g, 50.761 mmol), tri(o-tolyl)phosphine (0.92 g, 3.046 mmol), tributyl tin methoxide (24.4 mL, 76.14 mmol) and Isopropenyl acetate (8.49 mL, 78.17 mmol) in toluene (200 mL) was degassed under nitrogen for 15 minutes. Then palladium (II) chloride (0.63 g, 3.55 mmol) was added to this reaction mixture and continue to stir at 100° C. for 16 hours. Completion of the reaction was monitored by TLC (10% EA in Hexane). Upon completion, the reaction mixture was cooled to RT and concentrated under reduced pressure. The residue was filtered through celite bed and washed with water (100 mL) and DCM (100 mL). The reaction mixture was extracted with DCM twice (2×200 ml) and washed with water followed by brine solution. Combined organic layer was dried over anhydrous sodium sulphate, solvent was removed under vacuum and purified by silica gel column chromatography using ethyl acetate/hexane (20:80 v/v) as eluent to afford pure 1-(benzofuran-6-yl)propan-2-one (15-2) as light yellow liquid (7.0 g, 79%). 1H NMR (400 MHz, DMSO) δ 7.94 (d, J=2.0 Hz, 1H), 7.58 (d, J=7.92 Hz, 1H), 7.42 (s, 1H), 7.07 (d, J=7.84 Hz, 1H), 6.92 (d, J=1.12 Hz, 1H), 3.86 (s, 2H), 2.13 (s, 3H). LCMS: (ES) C₁₁H₁₀O₂ requires 174, found 175 [M+H]⁺.

Step 2: To a stirred solution of 1-(benzofuran-6-yl)propan-2-one (15-2) (5.5 g, 31.60 mmol) in THE (80 ml) was added Ti(OEt)₄ (23.20 mL, 110 mmol) followed by 2-methylpropane-2-sulfinamide (R)(dissolved in 5 ml THF) (3.82 g, 31.60) and the reaction mixture was allowed to stir at 70° C. for 12h. Completion of the reaction was monitored by TLC (50% EA in Hexane). The reaction mixture was cooled to 0° C. and NaBH4 (4.8 g, 126.4 mmol) was added to it at −45° C. and then it was allowed to stir at −45° C. for 2.5h. Completion of the reaction was observed in TLC (50% EA in Hexane) and crude LCMS. The reaction mixture was taken to RT and then it was quenched with methanol and Saturated NaCl solution (white precipitation observed). It was filtered through celite bed, washed the celite bed with methanol and DCM then the solvent was evaporated under vacuum to remove the volatiles. Then the reaction mixture was extracted with EA twice (2×200 ml) and washed with water followed by brine solution. Combined organic layer was dried over anhydrous sodium sulphate, solvent was removed under vacuum to afford the crude (R)-N-((R)-1-(benzofuran-6-yl)propan-2-yl)-2-methylpropane-2-sulfinamide (15-3) (8.0 g), which was used for next step without further purification. 1H NMR (400 MHz, DMSO) δ 7.92 (d, J=1.96 Hz, 1H), 7.56 (d, J=7.84 Hz, 1H), 7.44 (s, 1H), 7.11 (d, J=8.08 Hz, 1H), 6.90 (d, J=1.04 Hz, 1H), 4.98 (d, J=6.0 Hz, 1H), 3.49 (m, 1H), 3.08 (m, 1H), 2.79 (m, 1H), 1.08 (m, 12H). LCMS: (ES) C15H21NO2S, requires 279, found 280 [M+H]⁺.

Step 3: To a stirred solution of crude (R)-N-((R)-1-(benzofuran-6-yl)propan-2-yl)-2-methylpropane-2-sulfinamide (15-3) (8.0 g, 28.67 mmol) in THE (100 mL), NaH (60%) (2.2 g, 57.34 mmol) at 0° C. was added portion-wise then the reaction mixture was stirred at 0° C. for 30 min after that Iodomethane (3.54 mL, 57.34 mmol) was added to it and the reaction mixture was stirred at RT for 12h. Completion of the reaction was monitored by TLC (20% EA in Hexane). Upon completion, the reaction mixture was diluted with cold water (100 mL) extracted with EA twice (2×200 ml) and organic layer was washed with NaHCO3 solution (100 mL) followed by brine solution. Combined organic layer was dried over anhydrous sodium sulphate, solvent was removed under vacuum and purified by silica gel column chromatography using 15-20% ethyl acetate hexane to afford pure (R)-N-((R)-1-(benzofuran-6-yl)propan-2-yl)-N,2-dimethylpropane-2-sulfinamide (15-4) (4.0 g, 47%) as a colorless sticky solid. 1H NMR (400 MHz, DMSO) δ 7.91 (d, J=2.04 Hz, 1H), 7.56 (d, J=7.92 Hz, 1H), 7.42 (s, 1H), 7.10 (d, J=8.04 Hz, 1H), 6.90 (d, J=1.36 Hz, 1H), 3.59 (m, 1H), 2.95 (dd, J=13.42 Hz 1H), 2.84 (dd, J=13.38 Hz 1H), 2.51 (s, 3H), 1.10 (d, J=6.68 Hz, 3H), 1.02 (S, 9H). LCMS: (ES) C16H23NO2S, requires 293, found 294 [M+H]⁺.

Step 4: To a stirred solution of (R)-N-((R)-1-(benzofuran-6-yl)propan-2-yl)-N,2-dimethylpropane-2-sulfinamide (15-4) (9.4 g, 32.03 mmol) in 1, 4 dioxane (60 mL) was added 4(M) HCl in 1, 4 dioxane (30.0 mL) at 0° C. and the resulting reaction mixture was allowed to stir at room temperature for 5h. Upon completion of reaction (monitored by TLC, 10% EA in Hexane), the solvent were evaporated and the residue was dissolved in methanol and diethyl ether was added to it for precipitation, finally filter to get pure (R)-1-(benzofuran-6-yl)-N-methylpropan-2-amine hydrochloride (R-6-MAPB) (6.1 g, 84%) as white solid. 1H NMR (400 MHz, DMSO) δ 9.00 (bs, 2H), 7.96 (d, J=2.08 Hz, 1H), 7.62 (d, J=7.92 Hz, 1H), 7.53 (s, 1H), 7.16 (d, J=7.52 Hz, 1H), 6.93 (d, J=1.48 Hz, 1H), 3.41 (bs, 1H), 3.30 (dd, J=13.28 Hz, 1H), 2.80 (dd, J=13.2 Hz, 1H), 2.56 (s, 3H), 1.12 (d, J=6.48 Hz, 3H). LCMS: (ES) C12H16ClNO, requires 189, found 190 [M+H]⁺. HPLC: Purity (λ 250 nm): 99.58%.

### Synthesis 23. Synthesis of (S)-1-(benzofuran-6-yl)-N-methylpropan-2-amine (S-6-MAPB)

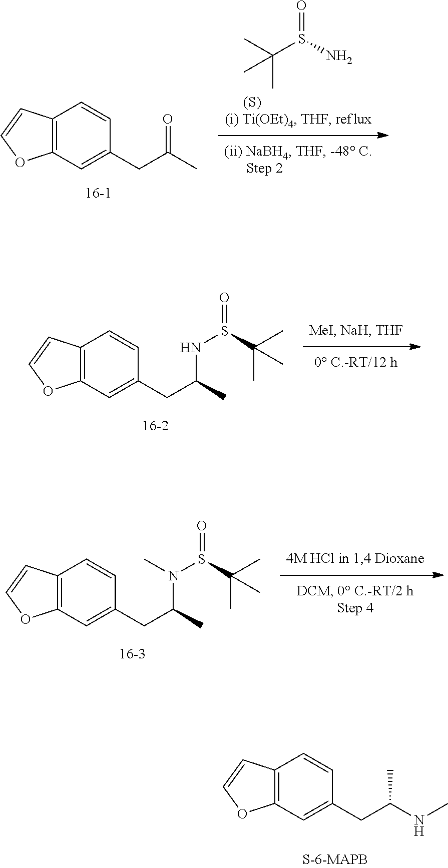

Step 1: To a stirred solution of 1-(benzofuran-6-yl)propan-2-one (16-1) (5 g, 28.70 mmol, 1 eq.) in dry THF (100 mL) was added Ti(OEt)₄ (21.06 mL, 100.45 mmol, 3.5 eq.) and (S)-2-methylpropane-2-sulfinamide (3.47 g, 28.73 mmol, 1 eq.) (dissolved in 20 mL dry THF) and the resulting reaction mixture was allowed to stir at 70° C. for 12 h. Upon completion, monitored by TLC (50% EA in Hexane), the reaction mixture was cooled to 0° C., gradually to −48° C. and NaBH₄ (4.34 g, 114.81 mmol, 4 eq.) (dissolved in 20 mL dry THF) was added into the reaction mixture at −48° C. and the resulting reaction mixture was allowed to stir at −48° C. for 3 h. Upon completion, monitored by TLC (50% EA in Hexane), the reaction mixture was taken to room temperature and was quenched with Methanol and saturated NaCl solution (until white precipitate observed). The reaction mixture was then filtered through celite bed, washed the celite bed with methanol (2×100 ml) and ethyl acetate (2×100 mL), and evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (S)-N-((S)-1-(benzofuran-6-yl)propan-2-yl)-2-methylpropane-2-sulfinamide (16-2) as yellow sticky gum (7.5 g, 93%). Crude 1H NMR (400 MHz, DMSO-d₆) δ 7.92 (d, J=2.08 Hz 1H), 7.56 (d, J=7.92 Hz, 1H), 7.44 (s, 1H), 7.11 (d, J=7.96 Hz, 1H), 6.90 (d, J=1.84 Hz, 1H), 4.96 (d, J=6.08 Hz, 1H), 3.30 (m, 1H), 3.08 (m, 1H), 2.80 (m, 1H), 1.10 (m, 9H), 1.08 (m, 3H). LCMS: (ES) C₁₅H₂₁NO₂S, requires 279, found 280 [M+H]⁺.

Step 2: To a stirred solution of (S)-N-((S)-1-(benzofuran-6-yl)propan-2-yl)-2-methylpropane-2-sulfinamide (16-2) (8 g, 28.67 mmol, 1 eq.) in dry THF (60 mL) (In a sealed tube) was added NaH (60%) (2.28 g, 57.26 mmol, 2 eq.) at 0° C. and the resulting reaction mixture was allowed to stir at 0° C. for 30 min. Then Iodomethane (3.56 mL, 57.26 mmol, 2 eq.) was added at 0° C. and the resulting reaction mixture was allowed to stir at room temperature for 12h. Upon completion, monitored by TLC (50% EA in Hexane), the reaction mixture was quenched with ice water, extracted with ethyl acetate (2×200 ml), washed with saturated ammonium chloride solution followed by brine solution. Combined organic layer was dried over anhydrous sodium sulphate, solvent was removed under vacuum and purified by silica gel column chromatography using ethyl acetate/hexane (50:50 v/v) as eluent to afford (S)-N-((S)-1-(benzofuran-6-yl)propan-2-yl)-N,2-dimethylpropane-2-sulfinamide (16-3) as light yellow gum (4.5 g, 53%). 1H NMR (400 MHz, DMSO-d₆) δ 7.92 (d, J=1.84 Hz, 1H), 7.56 (d, J=7.84 Hz, 1H), 7.42 (s, 1H), 7.10 (d, J=7.96 Hz, 1H), 6.90 (S, 1H), 3.57 (d, J=7.32 Hz, 1H), 2.92 (m, 1H), 2.84 (m, 1H), 2.51 (s, 3H), 1.10 (d, J=6.6 Hz, 3H), 1.02 (s, 9H). LCMS: (ES) C₁₆H₂₃NO₂S, requires 293, found 294 [M+H]⁺.

Step 3: To a stirred solution of (S)-N-((S)-1-(benzofuran-6-yl)propan-2-yl)-N,2-dimethylpropane-2-sulfinamide (16-3) (5.4 g, 18.40 mmol, 1 eq.) in dry DCM (45 mL) was added 4(M) HCl in 1,4 dioxane (90 mL) at 0° C. and the resulting reaction mixture was allowed to stir at room temperature for 2h. Upon completion of reaction (monitored by TLC, 30% EA in Hexane), the solvent was evaporated, and the crude was washed twice with diethyl ether (2×100 ml) and pentane and dried under vacuum to afford (S)-1-(benzofuran-6-yl)-N-methylpropan-2-amine hydrochloride S-6-MAPB (3.5 g, 84%) as white solid. ¹HNMR(400 MHz, DMSO-d₆) δ 9.01 (bs, 2H), 7.96 (d, J=2.04 Hz, 1H), 7.62 (d, J=7.88 Hz, 1H), 7.53 (s, 1H), 7.16 (d, J=7.88 Hz, 1H), 6.93 (d, J=1.64 Hz, 1H), 3.44 (bs, 1H), 3.30 (q, 1H), 2.80 (m, 1H), 2.56 (s, 3H), 1.12 (d, J=6.48 Hz, 3H). LCMS: (ES) C₁₂H₁₅NO, requires 189, found 190 [M+H]⁺. HPLC:Purity (λ 200 nm): 99.61%.

### Synthesis 24: Synthesis of (R)-1-(benzofuran-6-yl)-N-methylbutan-2-amine hydrochloride (R-6-MBPB)

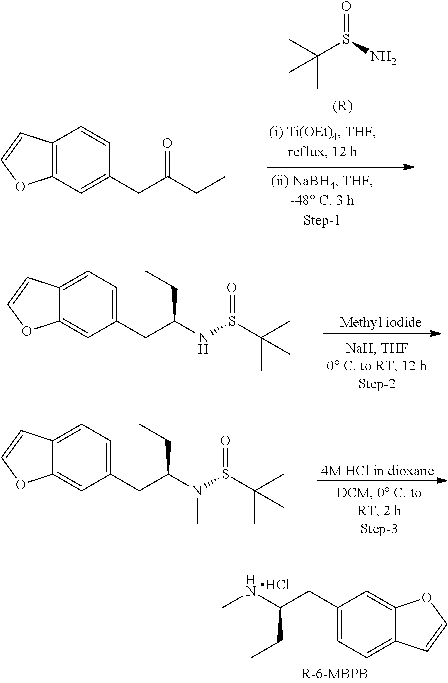

### Step-1

To a stirred solution of 1-(benzofuran-6-yl)butan-2-one (500 mg, 2.656 mmol, 1.0 equiv.) in THE (20 mL) was added Ti(OEt)₄ (2.0 mL, 9.297 mmol, 3.5 equiv.) and (R)-2-methylpropane-2-sulfinamide (dissolved in 5 mL THF) (321 mg, 2.656 mmol, 1.0 equiv.). Then the reaction mixture was allowed to stir at 70° C. for 12 h. After completion of the reaction (monitored by TLC, 50% EA in Hexane). The reaction mixture was cooled to 0° C. and NaBH4 (400 mg, 10.625 mmol, 4.0 equiv.) was added into it at −48° C. and the reaction mixture was allowed to stir at −45° C. for 3h. TLC (50% EA-Hexane) showed the formation of a new polar spot. Crude LCMS analysis showed formation of the desired product. The reaction mixture was taken to RT and then it was quenched with Methanol and Sat. NaCl solution (White Precipitate observed). The reaction mixture was filtered through celite bed, then washed with Methanol and DCM. Collect the organic layer and evaporated under vacuo to remove the volatiles. Then the reaction mixture was diluted with ethyl acetate, washed with water, and brine. Collect the organic layer, dried over anhydrous sodium sulphate and concentrated under vacuo to get the crude compound ((R)-N-(R)-1-(benzofuran-6-yl)butan-2-yl)-2-methylpropane-2-sulfinamide (900 mg). The crude compound was used in the next step without further purification. LCMS: Rt 1.98 min. MS (ES) C₁₆H₂₃NO2S requires 293, found 294 [M+H]⁺. Mass of the other isomer was observed in LCMS.

### Step-2

To a stirred solution of ((R)-N-(R)-1-(benzofuran-6-yl)butan-2-yl)-2-methylpropane-2-sulfinamide (900 mg, 3.06 mmol, 1 equiv.) (Sealed tube) in THE was added NaH (420 mg, 10.22 mmol, 3.0 equiv.) at 0° C. After that methyl iodide (0.85 mL, 13.63 mmol, 4 equiv.) was added to this reaction mixture. The reaction mixture was stirred at RT for 12 h. After completion of the reaction, the reaction mixture was quenched with ice-cold water and extracted with DCM. The organic part was concentrated, and the crude material was purified by silica gel column chromatography using ethyl acetate/hexane (10:90 v/v) as eluent to afford (R)-N-(R)-1-(benzofuran-6-yl)butan-2-yl)-N,2-dimethylpropane-2-sulfinamide as sticky gum (280 mg, 27%). ¹H NMR (400 MHz, DMSO) δ 7.92 (d, J=2 Hz, 1H), 7.56 (d, J=7.88 Hz, 1H), 7.41 (s, 1H), 7.10 (d, J=7.76 Hz, 1H), 6.90 (s, 1H), 2.94-2.82 (m, 2H), 2.45 (s, 3H), 1.57-1.54 (m, 1H), 1.40-1.35 (m, 1H), 1.23 (m, 1H), 1.08 (s, 9H), 0.86 (t, J=7.28 Hz, 3H). LCMS: Rt 1.88 min. MS (ES) C₁₇H₂₅NO2S requires 307, found 308 [M+H]⁺.

### Step-3

To a stirred solution of (R)-N-(R)-1-(benzofuran-6-yl)butan-2-yl)-N,2-dimethylpropane-2-sulfinamide (3) (100 mg, 0.325 mmol, 1.0 equiv.) in DCM was added 4M HCl in dioxane (0.5 mL) at 0° C. The reaction mixture was stirred at RT for 2 h. After completion of the reaction, evaporated the solvent in vacuo and solid was formed which was washed with ether to afford (R)-1-(benzofuran-6-yl)-N-methylbutan-2-amine hydrochloride as a white solid (52 mg, 78.65%). ¹H NMR (400 MHz, DMSO) δ 8.96-8.88 (bs, 1H), 8.81-8.75 (bs, 1H), 7.97 (d, J=2.04 Hz, 1H), 7.62 (d, J=7.92 Hz, 1H), 7.57 (s, 1H), 7.19 (d, J=7.96 Hz, 1H), 6.94 (d, J=1.44 Hz, 1H), 3.38-3.35 (m, 1H), 3.20-3.15 (dd, J=5.12 Hz, 5.20 Hz, 1H), 2.93-2.88 (m, 1H), 2.55 (s, 3H), 1.61-1.49 (m, 2H), 0.93 (t, J=7.44 Hz, 3H). LCMS: Rt 1.98 min. MS (ES) C₁₃H₁₈ClNO requires 203, found 204 [M+H]⁺. HPLC: Rt 4.28 min, Purity (λ 250 nm): 98.09%, chiral purity: Rt 6.24 min, 99.17%, ee: 98.34.

### Synthesis 25: Synthesis of (S)-1-(benzofuran-6-yl)-N-methylbutan-2-amine hydrochloride (S-6-MBPB)

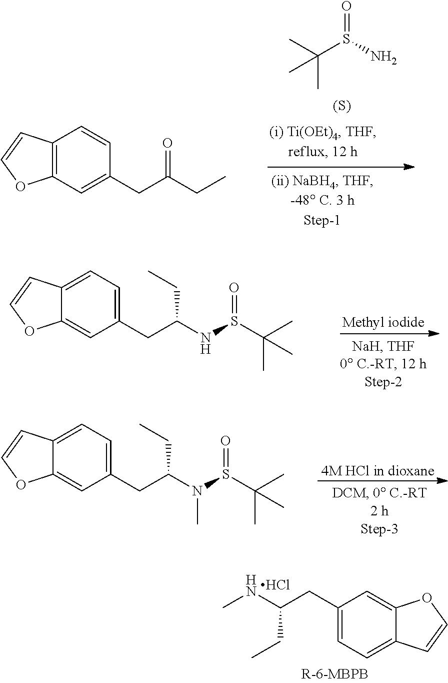

### Step-1

To a stirred solution of 1-(benzofuran-6-yl) butan-2-one (500 mg, 2.656 mmol, 1.0 equiv.) in dry THF (20 mL) was added Ti(OEt)₄ (2.0 mL, 9.297 mmol, 3.5 equiv.) and (S)-2-methylpropane-2-sulfinamide(dissolved in 5 mL THF) (321 mg, 2.656 mmol, 1.0 equiv.) at RT. The resulting reaction mixture was continued to stir at 70° C. for 12h. TLC (50% EtOAc-Hex) monitoring showed the formation of polar spot. The reaction mixture was cooled to 0° C. and NaBH₄ (400 mg, 10.625 mmol, 4.0 equiv.) was added into the reaction mixture at −48° C. and the reaction mixture was allowed to stir at −45° C. for 3 h. After completion, (monitored by TLC, 50% EA-Hexane), the reaction mixture was taken to RT, quenched with Methanol and Saturated NaCl solution (White Precipitate observed). The reaction mixture was filtered through celite bed, washed the celite bed with Methanol and DCM, and evaporated under vacuo to remove the volatiles. Then the reaction mixture was taken in ethyl acetate, washed with water, followed by brine then dried over sodium sulphate, and concentrated under vacuo to get the crude compound (S)-N-((S)-1-(benzofuran-6-yl) butan-2-yl)-2-methylpropane-2-sulfinamide (900 mg) as a colorless sticky liquid, which was forwarded to the next step without purification. LCMS: Rt 3.53 min. MS (ES) C₁₆H₂₃NO2S requires 293, found 294 [M+H]⁺. Mass of other isomer observed in LCMS.

### Step-2

To a stirred solution of (S)-N-((S)-1-(benzofuran-6-yl) butan-2-yl)-2-methylpropane-2-sulfinamide (crude) (900 mg, 3.067 mmol, 1.0 equiv.) (Sealed tube) in dry THE (20.0 mL) was added (60%) NaH (370 mg, 9.202 mmol, 3.0 equiv.) at 0° C. After that methyl iodide (0.9 mL, 12.269 mmol, 4 equiv.) was added to the reaction mixture. The resulting reaction mixture was stirred at RT for 12 h. After completion (Monitored by TLC, 40% EA in Hex), the reaction mixture was quenched with cold water (30 mL) and extracted with ethyl acetate (200 mL) and then washed with NaCl solution. The collected organic layer was dried over sodium sulphate and concentrated under reduced pressure. The crude compound was purified by combi flash column chromatography eluted with 10%-15% ethyl acetate in hexane to afford (S)-N-((S)-1-(benzofuran-6-yl)butan-2-yl)-N,2-dimethylpropane-2-sulfinamide (280.0 mg) as a colorless sticky liquid. ¹H NMR (400 MHz, CDCl3) δ 7.56 (m, 1H), 7.50 (d, J=7.88 Hz, 1H), 7.31 (bs, 1H), 7.07 (d, J=7.84 Hz, 1H), 6.71 (bs, 1H), 3.31 (bs, 1H), 3.17-3.12 (m, 1H), 2.84-2.78 (m, 1H), 2.55 (s, 3H), 1.52-1.50 (m, 2H), 1.19 (s, 9H), 0.89-0.85 (m, 3H). LCMS: Rt 3.64 min. MS (ES) C₁₇H₂₅NO₂S requires 307, found 307.7 [M+H]⁺.

### Step-3

To a stirred solution of (S)-N-((S)-1-(benzofuran-6-yl) butan-2-yl)-N,2-dimethylpropane-2-sulfinamide (280 mg, 0.867 mmol, 1.0 equiv.) in DCM (5.0 mL) was added 4M HCl in dioxane (2.0 mL) at 0° C. The resulting reaction mixture was stirred at 0° C.-RT for 2 h. After completion (Monitoring by TLC, 20% EA in Hex), the excess solvent was evaporated under reduced pressure to get the crude, which was washed with diethyl ether and dried to afford S-6-MBPB (150.0 mg) as a white solid. ¹H NMR (400 MHz, DMSO-d₆) δ 9.05-8.90 (bm, 2H), 7.96 (d, J=1.96 Hz, 1H), 7.62-7.60 (d, J=7.96 Hz, 1H), 7.57 (s, 1H), 7.19 (d, J=7.84 Hz, 1H), 6.93 (d, J=1.16 Hz, 1H), 3.22-3.18 (dd, J=13.76 Hz, 4.96 Hz, 1H), 2.94-2.88 (m, 1H), 2.54 (s, 3H), 1.62-1.48 (m, 2H), 0.92 (t, J=7.44, 7.48 Hz, 3H). LCMS: Rt 1.98 min. MS (ES) C₁₃H₁₈ClNO requires 203, found 204 [M+H]⁺. HPLC: Rt 6.97 min. Purity (λ 210 nm): 98.11% Chiral purity: Rt 7.38: 97.56%, ee is 95.12.

### Synthesis 26: Synthesis of (S)-1-(benzofuran-5-yl)-N-methylbutan-2-amine hydrochloride (S-5-MBPB)

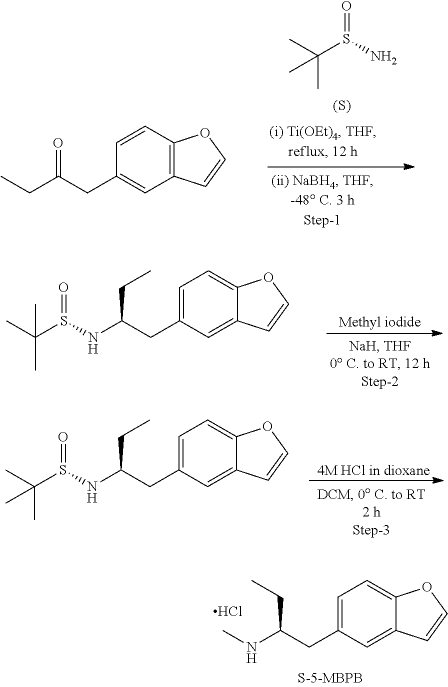

### Step-1

To a stirred solution of 1-(benzofuran-5-yl) butan-2-one (2 g, 10.625 mmol, 1 equiv.) in dry THF (10 mL) was added Ti(OEt)4 (7.797 mL, 37.189 mmol, 3.5 equiv.) and (S)-2-methylpropane-2-sulfinamide (1.288 g, 10.625 mmol, 1 equiv.) (dissolved in 10 mL dry THF) and the resulting reaction mixture was allowed to stir at 70° C. for 12 h. Upon completion, monitored by TLC (50% EA in Hexane), the reaction mixture was cooled to 0° C., gradually to −48° C. and NaBH4 (1.615 g, 42.501 mmol, 4 equiv.) was added into the reaction mixture at −48° C. and the resulting reaction mixture was allowed to stir at −48° C. for 3 h. Upon completion, monitored by TLC (50% EA in Hexane), the reaction mixture was slowly warm to room temperature and was quenched with Methanol and saturated NaCl solution (until a white precipitate was observed). The reaction mixture was then filtered through celite bed, washed the celite bed with methanol (2×100 mL) and ethyl acetate (2×100 mL), then evaporated under vacuum to remove the volatiles. Then the reaction mixture was diluted with ethyl acetate, washed with water, followed by brine solution. The combined organic layer was collected and dried over anhydrous sodium sulphate, the solvent was removed under vacuum to afford the crude (S)-N-((S)-1-(benzofuran-5-yl) butan-2-yl)-2-methylpropane-2-sulfinamide as yellow sticky gum (2.8 g, 89.81%). Proceed for the next step without further purification. LCMS: Rt 1.94 min. MS (ES) C16H23NO2S, requires 293, found 294 [M+H]⁺.

### Step-2

To a stirred solution of crude (S)-N-((S)-1-(benzofuran-5-yl) butan-2-yl)-2-methylpropane-2-sulfinamide (3.94 g, 13.441 mmol, 1 equiv.) in dry THF (40 mL) (In a sealed tube) was added NaH (60% in mineral oil) (0.968 g, 40.322 mmol, 3 equiv.) at 0° C. and the resulting reaction mixture was allowed to stir at 0° C. for 30 min. Then methyl iodide (3.347 mL, 53.763 mmol, 4 equiv.) was added at 0° C. and the resulting reaction mixture was allowed to stir at room temperature for 12h. Upon completion, monitored by TLC (50% EA in Hexane), the reaction mixture was quenched with ice water, extracted with ethyl acetate (2×200 mL), then washed with saturated ammonium chloride solution followed by brine solution. The combined organic layer was dried over anhydrous sodium sulphate, filtered and the solvent was removed under vacuum to get the crude which was purified by silica gel column chromatography using ethyl acetate/hexane (50:50 v/v) as eluent to afford (S)-N-((S)-1-(benzofuran-5-yl)butan-2-yl)-N,2-dimethylpropane-2-sulfinamide as light yellow gum (3.8 g, 91.96%). We can separate other minor isomer formed by column chromatography at this step. ¹H NMR (400 MHz, CDCl3) δ 7.58 (d, J=2.04 Hz, 1H), 7.41-7.39 (bs, 2H), 7.11-7.09 (dd, J=1.36 Hz, J=8.40 Hz, 1H), 6.70 (d, J=1.28 Hz, 1H), 3.31-3.28 (m, 1H), 3.14-3.09 (dd, J=4.16 Hz, 13.44 Hz, 1H), 2.55 (s, 3H), 1.55-1.48 (m, 2H), 1.19 (s, 9H), 0.89 (t, J=7.32 Hz, 7.36 Hz, 3H). LCMS: Rt 2.08 min. MS (ES) C17H25NO2S, requires 307, found 308 [M+H]⁺.

### Step-3

To a stirred solution of (S)-N-((S)-1-(benzofuran-5-yl) butan-2-yl)-N,2-dimethylpropane-2-sulfinamide (736 mg, 2.396 mmol, 1 equiv.) in dry DCM (10 mL) was added 4(M) HCl in 1,4 dioxane (5 mL) at 0° C. and the resulting reaction mixture was allowed to stir at room temperature for 2 h. After completion of the reaction, the solvent was evaporated and the crude was washed twice with diethyl ether (2×100 mL) and pentane and then dried under vacuum to afford S-5-MBPB (410 mg, 84.17%) as a white solid. ¹HNMR(400 MHz, DMSO-d6) δ 8.94-8.79 (bs, 2H), 7.99 (d, J=1.96 Hz, 1H), 7.57-7.55 (m, 2H), 7.24 (d, J=8.36 Hz, 1H), 6.93 (d, J=1.24 Hz, 1H), 3.19-3.14 (dd, J=4.96 Hz, J=13.76 Hz, 1H), 2.91-2.86 (q, 1H), 2.55 (s, 3H), 1.60-1.51 (m, 2H), 0.92 (t, J=7.44 Hz, 7.48 Hz, 3H) LCMS: Rt 1.39 min. MS (ES) C13H17NO, requires 203.13, found 204 [M+H]⁺. HPLC: Rt 4.75 min. Purity (λ 260 nm): 96.54%, chiral purity: Rt 3.18 min, 99.05%, ee: 98.11.

### Synthesis 27 Synthesis of (R)-1-(benzofuran-5-yl)-N-methylbutan-2-amine (R-5-MBPB)

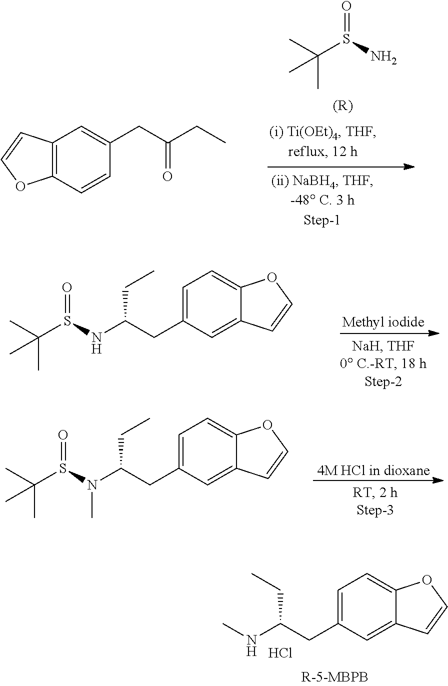

### Step-1

To a stirred solution of 1-(benzofuran-5-yl) butan-2-one (2.0 g, 10.625 mmol, 1.0 equiv.) in THF (40.0 mL) was added Ti(OEt)₄ (7.7 mL, 37.19 mmol, 3.5 equiv.) and (R)-2-methylpropane-2-sulphinamide (dissolved in 5 ml THF) (1.3 g, 10.6 mmol, 1.0 equiv.) then the reaction mixture was allowed to stir at 70° C. for 12h. TLC (50% EA in Hexane) monitoring showed the formation of new polar spot. The reaction mixture was cooled to 0° C. and NaBH₄ (1.7 g, 42.5 mmol, 4.0 equiv.) was added into the reaction mixture at −45° C. and the reaction mixture was allowed to stir at −45° C. for 3 hrs. TLC (50% EA-Hexane) showed formation of new polar spot. Crude LCMS showed the formation of the desired product. The reaction mixture was taken to RT and it was quenched with Methanol and Sat. NaCl solution (White Precipitate observed). The reaction mixture was filtered through a celite bed, washed the celite bed with Methanol and DCM, and evaporated under vacuo to remove the volatiles. Then it was diluted with ethyl acetate, washed with water, followed by brine then dried over sodium sulphate, and concentrated under vacuo to get the crude (R)-N-((R)-1-(benzofuran-5-yl)butan-2-yl)-2-methylpropane-2-sulfinamide (2 g, 64%), which was used in next step without further purification. LCMS: Rt 1.94 min. MS (ES) C₁₆H₂₃NO2S requires 293, found 294 [M+H]⁺.

### Step-2

To a stirred solution of (R)-N-((R)-1-(benzofuran-5-yl)butan-2-yl)-2-methylpropane-2-sulfinamide (2.0 gm, 6.816 mmol, 1.0 equiv.) (Sealed tube) in THE (20 mL) was added NaH (60%) (818 mg, 20.448 mmol, 3.0 equiv.) Portion-wise at 0° C. and stirred at same temperature for 30 min. Then methyl iodide (2 mL, 27.264 mmol, 4.0 equiv.) was added and the resulting reaction mixture was stirred at RT for 16 h. After completion (monitoring by TLC), the reaction mixture was quenched with cold water (50 mL) and Ethyl acetate (100 mL). The organic part was collected and washed with sat. NaHCO₃ (20 mL) solution followed by brine. The organic layer was collected and dried over anhydrous sodium sulfate, filtered and evaporated under reduced pressure to get the crude which was purified by silica gel (100-200 mesh) column chromatography and elute with 15% ethyl acetate-hexane to get the desired product (900 mg, 43%) as a colorless sticky gum. ¹H NMR (400 MHz, DMSO-d₆) δ 7.94-7.92 (m, 1H), 7.50-7.45 (m, 2H), 7.13 (d, J=8.36, 1H), 6.89 (s, 1H), 3.27-3.25 (m, 1H), 2.92-2.79 (m, 2H), 2.45 (m, 3H), 1.58-1.50 (m, 1H), 1.42-1.35 (m, 1H), 1.08 (bs, 9H), 0.83 (t, J=7.32 Hz, 3H). LCMS: Rt 2.03 min. MS (ES) C₁₇H₂₅NO₂S requires 307, found 308 [M+H]⁺.

### Step-3

To a stirred solution of (R)-N-((R)-1-(benzofuran-5-yl) butan-2-yl)-N,2-dimethylpropane-2-sulfinamide (900 mg, 2.927 mmol, 1 equiv.) in dry DCM (10 mL) was added 4M HCl in 1,4 dioxane (4.0 mL) at 0° C. and then the resulting reaction mixture was allowed to stir at room temperature for 2h. Upon completion of the reaction, the solvent was evaporated, and the crude was washed twice with diethyl ether (2×20 ml) and pentane and dried under vacuum to afford R-5-MBPB (550 mg, 78%) as off white solid. ¹HNMR (400 MHz, DMSO-d₆) δ 8.85-8.72 (bm, 2H), 7.99 (d, J=2 Hz, 1H), 7.57-7.55 (m, 2H), 7.24 (d, J=9.52 Hz, 1H), 6.93 (d, J=1.8 Hz, 1H), 3.17-3.13 (dd, J=4.8 Hz, 5.2 Hz 1H), 2.91-2.85 (q, 1H), 2.56 (bs, 3H), 1.61-1.49 (m, 2H), 1.04 (s, 1H), 0.89 (t, J=7.44 Hz, 7.52 Hz, 3H). LCMS: Rt 1.94 min. MS (ES) C₁₃H₁₇NO requires 203, found 204 [M+H]⁺. HPLC: Rt 6.51 min. Purity (λ 220 nm): 95.08%, chiral purity: Rt 3.82 min. 99.51%, ee 99.03.

### Synthesis 28: Synthesis of (S)-1-(benzofuran-5-yl)-N-methylpropan-2-amine hydrochloride (S-5-MAPB)

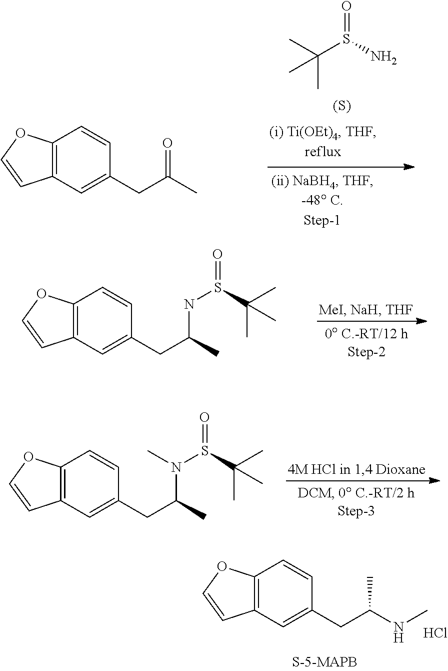

Actual stereochemistry mentioned here based on literature report J. Med. Chem. 2017, 60, 3958-3978.

### Step-1

To a stirred solution of 1-(benzofuran-5-yl)propan-2-one (5 g, 28.70 mmol, 1 eq.) in dry THF (100 ml) was added Ti(OEt)4 (21.06 ml, 100.45 mmol, 3.5 eq.) and (S)-2-methylpropane-2-sulfinamide (3.47 g, 28.73 mmol, 1eq.) (dissolved in 20 ml dry THF) and the resulting reaction mixture was allowed to stir at 70° C. for 12 hrs. Upon completion (monitored by TLC, 50% EA in Hexane), the reaction mixture was cooled to 0° C., gradually to −48° C. and NaBH4 (4.34 g, 114.81 mmol, 4 eq.) (dissolved in 20 ml dry THF) was added into the reaction mixture at −48° C. and the resulting reaction mixture was allowed to stir at −48° C. for 3 hrs. Upon completion (monitored by TLC, 50% EA in Hexane), the reaction mixture was taken to room temperature and was quenched with Methanol and Sat. NaCl solution (until white precipitate observed). The reaction mixture was then filtered through celite bed, washed with methanol (2×100 ml) and ethyl acetate (2×100 ml), evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (S)-N-((S)-1-(benzofuran-5-yl)propan-2-yl)-2-methylpropane-2-sulfinamide as yellow sticky gum (6.5 g, 81%). Crude ¹H NMR (400 MHz, DMSO-d6) δ 7.94 (d, J=7.8 Hz, 1H), 7.50 (m, 2H), 7.14 (m, 1H), 6.90 (d, J=6.36 Hz, 1H), 6.90 (d, J=6.36 Hz, 1H), 4.97 (d, J=5.96 Hz, 1H), 3.48 (m, 1H), 3.08 (m, 1H), 2.76 (m, 1H), 1.18 (m, 12H). LCMS: Rt 1.83 min. MS (ES) C15H21NO2S requires 279, found 280 [M+H]⁺.

### Step-2

To a stirred solution of (S)-N-((S)-1-(benzofuran-5-yl)propan-2-yl)-2-methylpropane-2-sulfinamide (7 g, 25 mmol, 1 eq.) in dry THE (50 mL) (In a sealed tube) was added NaH (60%) (2 g, 50 mmol, 2 eq.) at 0° C. and the resulting reaction mixture was allowed to stir at 0° C. for 30 min. Then Iodomethane (3.11 ml, 50 mmol, 2 eq.) was added at 0° C. and the resulting reaction mixture was allowed to stir at room temperature for 12h. Upon completion (monitored by TLC, 50% EA in Hexane), the reaction mixture was quenched with ice water, extracted with ethyl acetate (2×200 ml), washed with saturated ammonium chloride solution, followed by brine solution. Combined organic layer was dried over anhydrous sodium sulphate, solvent was removed under vacuum and purified by silica gel column chromatography using ethyl acetate/hexane (50:50 v/v) as eluent to afford (S)-N-((S)-1-(benzofuran-5-yl)propan-2-yl)-N,2-dimethylpropane-2-sulfinamide as light-yellow gum (4 g, 54%). ¹H NMR (400 MHz, DMSO-d6) δ 7.94 (s, 1H), 7.49 (t, J=8.4 Hz, 9.04 Hz, 2H), 7.14 (d, J=8.2, 1H), 6.89 (s, 1H), 3.55 (m, 1H), 2.92 (m, 1H), 2.88 (m, 1H), 2.51 (s, 3H), 1.27 (m, 3H), 1.07 (S, 9H). LCMS: Rt 1.91 min. MS (ES) C16H23NO2S requires 293, found 294 [M+H]⁺.

### Step-3

To a stirred solution of (S)-N-((S)-1-(benzofuran-5-yl)propan-2-yl)-N,2-dimethylpropane-2-sulfinamide (3) (7 g, 23.89 mmol, 1 eq.) in dry DCM (35 mL) was added 4M-HCl in 1,4 dioxane (70 mL) at 0° C. and the resulting reaction mixture was allowed to stir at room temperature for 2h. Upon completion of reaction (monitored by TLC, 30% EA in Hexane), the solvent was evaporated, and the crude was washed twice with diethyl ether (2×60 ml) and pentane and dried under vacuum to afford (S)-1-(benzofuran-5-yl)-N-methylpropan-2-amine hydrochloride (Compound-9S) (5 g, 97%) as off white solid. ¹HNMR (400 MHz, DMSO-d6) δ 9.06 (bs, 2H), 7.99 (d, J=1.88 Hz, 1H), 7.57 (m, 2H), 7.21 (d, J=8.28 Hz, 1H), 6.93 (d, J=1.32 Hz, 1H), 3.33 (m, 1H), 3.26 (m, 1H), 2.77 (q, 1H), 2.56 (s, 3H), 1.11 (d, J=6.4 Hz, 3H), LCMS: Rt 1.33 min. MS (ES) C12H15NO requires 189, found 190 [M+H]⁺. HPLC: Rt 5.73 min. Purity (λ 250 nm): 99.81%.

### Synthesis 29: Synthesis of (R)-1-(benzofuran-5-yl)-N-methylpropan-2-amine hydrochloride (R-5-MAPB)

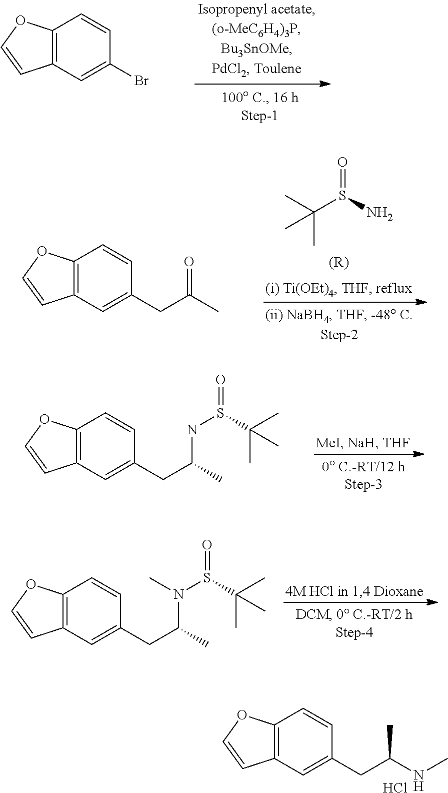

Actual stereochemistry mentioned here based on literature report J. Med. Chem. 2017, 60, 3958-3978.

### Step-1

To a stirred solution of 5-bromobenzofuran (20 g, 101.52 mmol, 1 eq.) in dry Toluene (400 ml) was added tri(o-tolyl)phosphine (1.84 g, 6.091 mmol, 0.06 eq.), tributyl tin methoxide (48.89 mL, 152.28 mmol, 1.5 eq.) and Isopropenyl acetate (16.99 mL, 156.34 mmol, 1.54 eq.) and the resulting reaction mixture was degassed under nitrogen for 15 minutes. Then palladium (II) chloride (1.26 g, 7.10 mmol, 0.07 eq.) was added to the reaction mixture and the resulting reaction mixture was heated to 100° C. for 16 hrs. Upon completion, monitored by TLC (10% EA in Hexane), the reaction mixture was filtered through celite bed, extracted with ethyl acetate (2×400 ml), washed with water, followed by saturated potassium fluoride solution, and brine solution. Combined organic layer was dried over anhydrous sodium sulphate, solvent was removed under vacuum and purified by silica gel column chromatography using ethyl acetate/hexane (10:90 v/v) as eluent to afford 1-(benzofuran-5-yl)propan-2-one as light yellow gum (17 g, 96%).

¹H NMR (400 MHz, DMSO-d6) δ 7.96 (d, J=2.08 Hz, 1H), 7.53 (d, J=8.48 Hz, 1H), 7.46 (s, 1H), 7.13 (dd, J=1.52 Hz, 8.44 Hz, 1H), 6.92 (d, J=0.76 Hz, 1H), 3.83 (s, 2H), 2.12 (s, 3H). LCMS: Rt 1.74 min. MS (ES) C11H₁₀O₂ requires 174, found 175 [M+H]⁺.

### Step-2

To a stirred solution of 1-(benzofuran-5-yl)propan-2-one (9 g, 51.66 mmol, 1eq.) in dry THE (150 ml) was added Ti(OEt)4 (37.91 ml, 180.82 mmol, 3.5eq.) and (R)-2-methylpropane-2-sulfinamide (6.26 g, 51.66 mmol, 1eq.) (dissolved in 30 ml dry THF) and the resulting reaction mixture was allowed to stir at 70° C. for 12 hrs. Upon completion, monitored by TLC (50% EA in Hexane), the reaction mixture was cooled to 0° C., gradually to −48° C. and NaBH4 (7.81 g, 206.65 mmol, 4 eq.) (dissolved in 30 ml dry THF) was added into the reaction mixture at −48° C. and the resulting reaction mixture was allowed to stir at −48° C. for 3 hrs. Upon completion, monitored by TLC (50% EA in Hexane), the reaction mixture was taken to room temperature and was quenched with Methanol and Sat NaCl solution (until white precipitate observed). The reaction mixture was then filtered through celite bed, washed with methanol (2×150 ml) and ethyl acetate (2×150 ml), evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (R)-N-((R)-1-(benzofuran-5-yl)propan-2-yl)-2-methylpropane-2-sulfinamide as yellow sticky gum (14 g, 96%). ¹H NMR (400 MHz, DMSO-d6) δ 7.94 (s, 1H), 7.48 (m, 2H), 7.15 (d, J=8.32 Hz, 1H), 6.89 (d, J=7.76 Hz, 1H), 4.97 (d, J=6.04 Hz, 1H), 3.48 (m, 1H), 3.07 (m, 1H), 2.76 (m, 1H), 1.09 (s, 12H), 1.08 (m, 3H) LCMS: Rt 1.87 min. MS (ES) C15H21NO2S requires 279, found 280 [M+H]⁺.

### Step-3

To a stirred solution of (R)-N-((R)-1-(benzofuran-5-yl)propan-2-yl)-2-methylpropane-2-sulfinamide (15 g, 53.57 mmol, 1 eq.) in dry THE (100 mL) (In a sealed tube) was added NaH (60%) (4.28 g, 107.14 mmol, 2 eq.) at 0° C. and the resulting reaction mixture was allowed to stir at 0° C. for 30 min. Then Iodomethane (6.7 ml, 107.14 mmol, 2 eq.) was added at 0° C. and the resulting reaction mixture was allowed to stir at room temperature for 12h. Upon completion, monitored by TLC (50% EA in Hexane), the reaction mixture was quenched with ice water, extracted with ethyl acetate (2×250 ml), washed with saturated ammonium chloride solution, followed by brine solution. Combined organic layer was dried over anhydrous sodium sulphate, solvent was removed under vacuum and purified by silica gel column chromatography using ethyl acetate/hexane (50:50 v/v) as eluent to afford (R)-N-((R)-1-(benzofuran-5-yl)propan-2-yl)-N,2-dimethylpropane-2-sulfinamide as light-yellow gum (8 g, 50.9%). ¹H NMR (400 MHz, DMSO-d6) δ 7.93 (s, 1H), 7.49 (m, 2H), 7.14 (d, J=7.4, 1H), 6.89 (s, 1H), 3.54 (m, 1H), 2.92 (m, 1H), 2.81 (m, 1H), 2.49 (s, 3H), 1.09 (d, J=6.64 Hz, 3H), 1.02 (s, 9H). LCMS: Rt 1.95 min. MS (ES) C16H23NO2S requires 293, found 294 [M+H]⁺.

### Step-4

To a stirred solution of (R)-N-((R)-1-(benzofuran-5-yl)propan-2-yl)-N,2-dimethylpropane-2-sulfinamide (10.5 g, 37.58 mmol, 1 eq.) in dry DCM (50 ml) was added 4M HCl in 1,4 dioxane (100 mL) at 0° C. and then the resulting reaction mixture was allowed to stir at room temperature for 2h. Upon completion of reaction (monitored by TLC, 30% EA in Hexane), the solvent were evaporated and the crude was washed twice with diethyl ether (2×60 ml) and pentane and dried under vacuum to afford R-5-MAPB (5.8 g, 81%) as off white solid. ¹HNMR (400 MHz, DMSO-d6) δ 9.00 (bs, 2H), 7.99 (d, J=1.6 Hz, 1H), 7.57 (m, 2H), 7.21 (d, J=7.8 Hz, 1H), 6.93 (s, 1H), 3.38 (bs, 1H), 3.25 (m, 1H), 2.77 (m, 1H), 2.56 (s, 3H), 1.11 (d, J=6.28 Hz, 3H). LCMS: Rt 1.32 min. MS (ES) C12H15NO requires 189, found 190 [M+H]⁺. HPLC: Rt 5.75 min. Purity (λ 210 nm): 99.26%.

### Synthesis 30: Synthesis of aF-5-MAPB (1-(benzofuran-5-yl)-3-fluoro-N-methylpropan-2-amine)

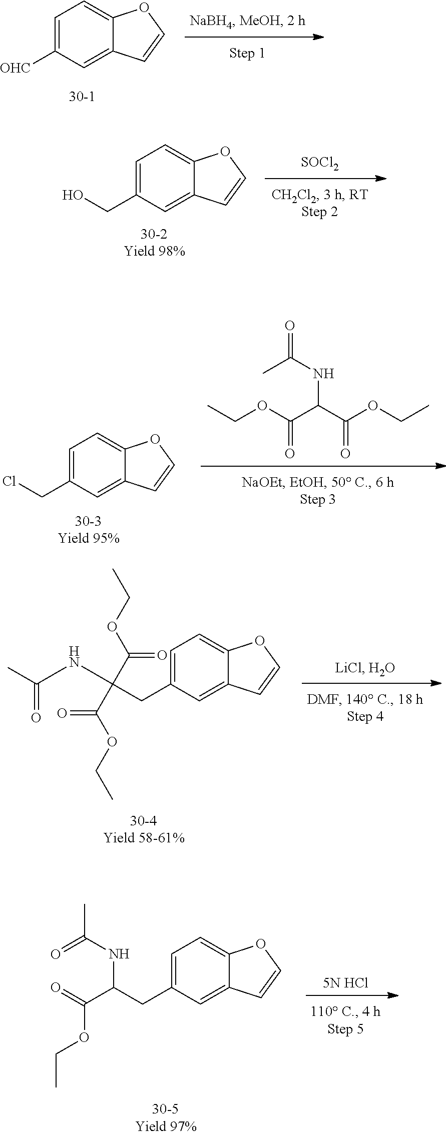

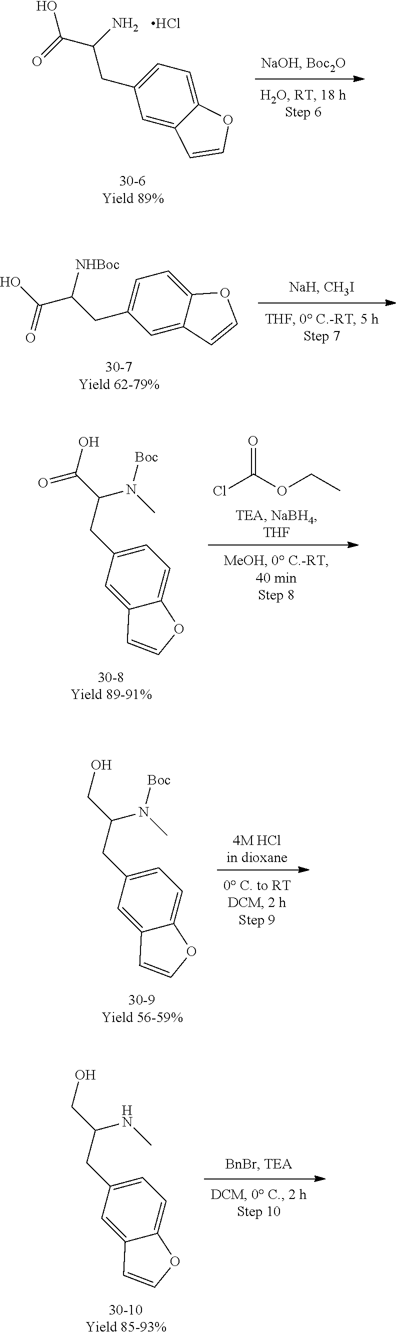

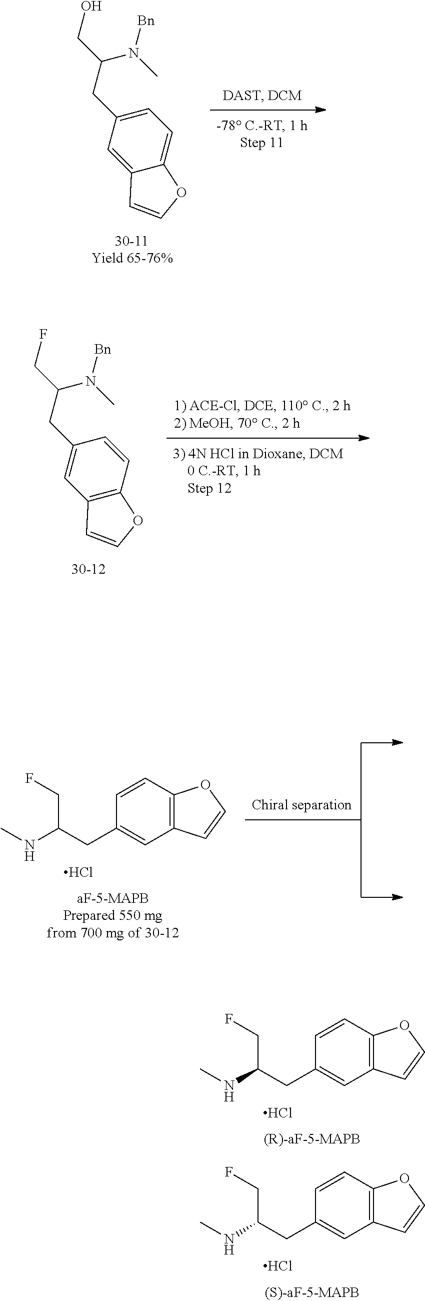

### Synthesis 31: Synthesis of aF-6-MAPB (1-(benzofuran-6-yl)-3-fluoro-N-methylpropan-2-amine)

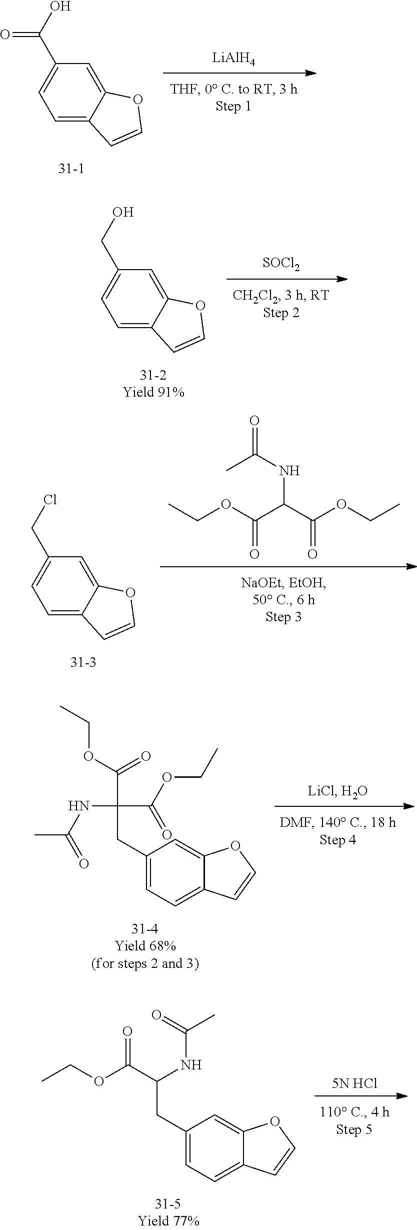

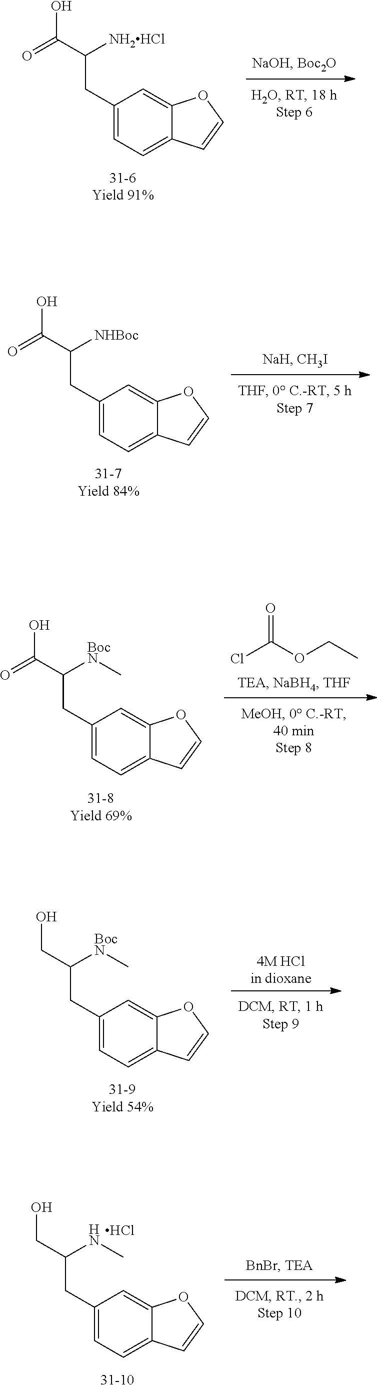

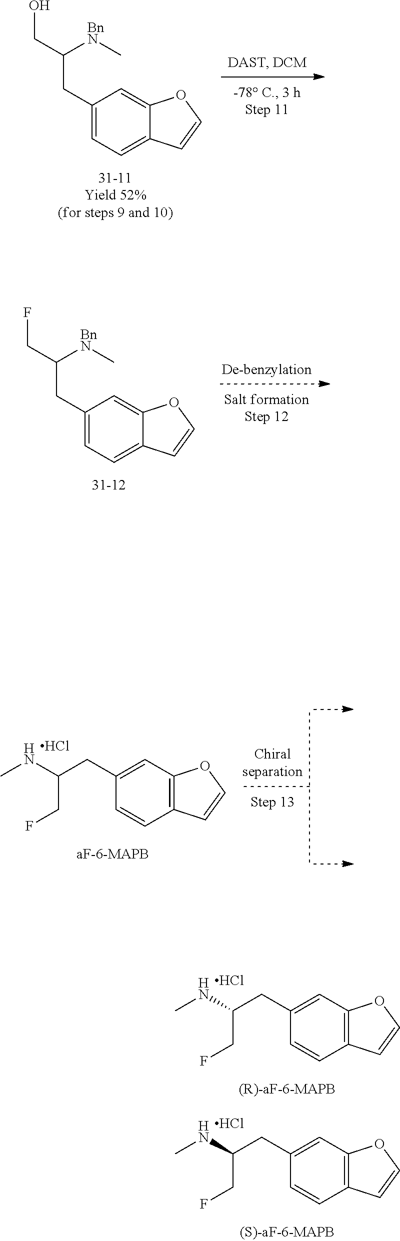

### EXAMPLE 3: nAChR α4β2 Receptor Agonism

An IonFlux™ automated patch-clamp system is used to measure activity of S-5-MAPB, R-5-MAPB at nAChR α4β2 receptors (Eurofins, cat. No. CYL3106) expressed in HEK-293 cells as described in Yehia & Wei, 2020, Current Protocols in Pharmacology, 88(1). Acetylcholine is used as a positive control. Results show that the compounds of the current invention are active as agonists, with enantioselective effects in which the R-enantiomers have greater potency.

### EXAMPLE 4: Serum Serotonin Concentrations to Index Drug Interactions with the

Serotonin Transporter (SERT, SLC6A4) Serum serotonin is measured using High Performance Liquid Chromatography and Fluorescence Detection. Venipuncture is used to collect at least 1 mL of sample, which is spun with serum frozen to below −20° C. within 2 hours of collection. Assay results show robust and enantioselective increases in serum serotonin, indicating that the S-enantiomers are more potent releasers of serotonin.

### EXAMPLE 5: Marble Burying Measure of Decreased Anxiety and Neuroticism

The marble burying test is a model of neophobia, anxiety, and obsessive-compulsive behavior that has been proposed to have predictive validity for the screening of novel antidepressants and anxiolytics. It is well established to be sensitive to the effects of SSRIs as well as serotonin releasers such as fenfluramine and MDMA (De Brouwer et al., Cognitive, Affective, and Behavioral Neuroscience, 2019, 19(1), 1-39).

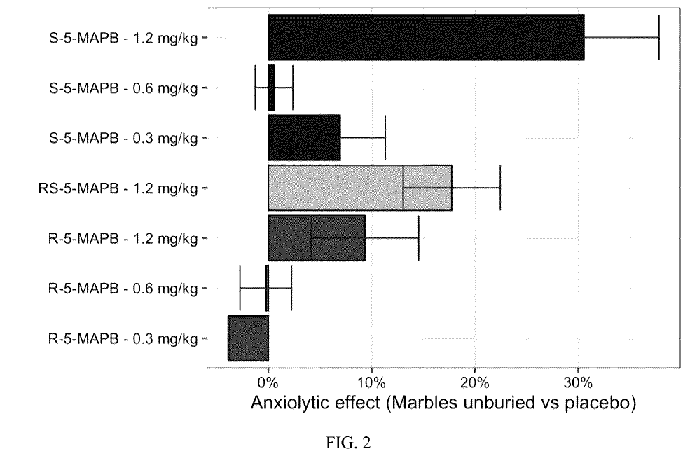

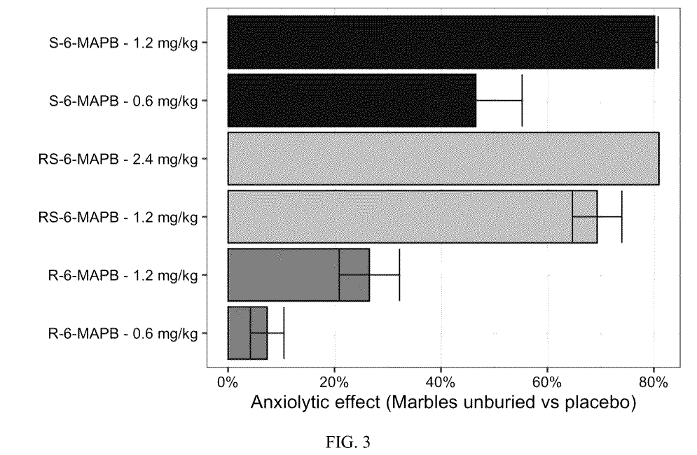

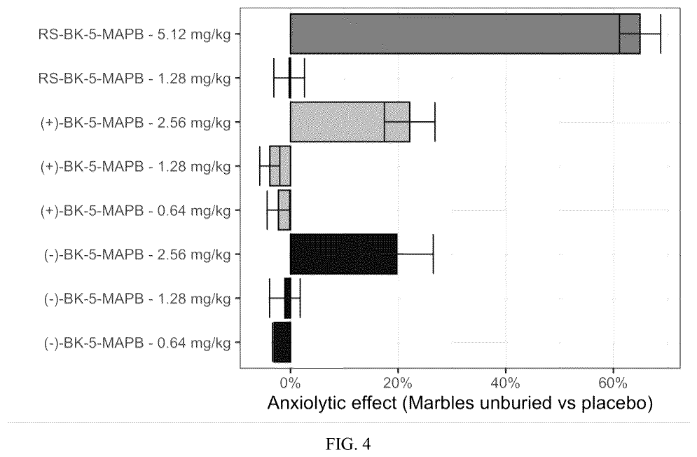

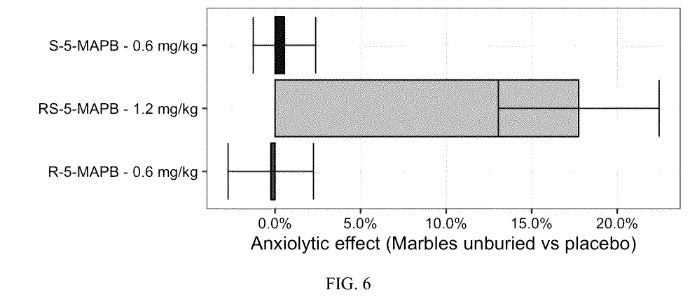

The test involved the placement of a standardized number of marbles gently onto the surface of a layer of bedding material within a testing arena. Mice were then introduced into the arena for a standardized amount of time and allowed to explore the environment. The outcome measure of the test was the number of marbles covered as scored by automatic scoring software or blinded observers. General locomotor activity, often operationalized as total distance traveled, was used as a control measure. Compounds that attenuate anxiety, neuroticism, or obsessive-compulsive behavior decrease marble burying. The racemates and individual enantiomers of 5-MAPB, 6-MAPB, BK-5-MAPB, and BK-5-MBPB were assessed with the marble burying assay. The results, which are shown graphically in FIG. 2 to FIG. 6, indicate that every tested compound had CNS modulating effects within 30 minutes. Every tested compound besides Bk-5-MAPB showed differences in activity between the two possible enantiomers. A strong non-additive interaction was also observed between the enantiomers of 5-MAPB.

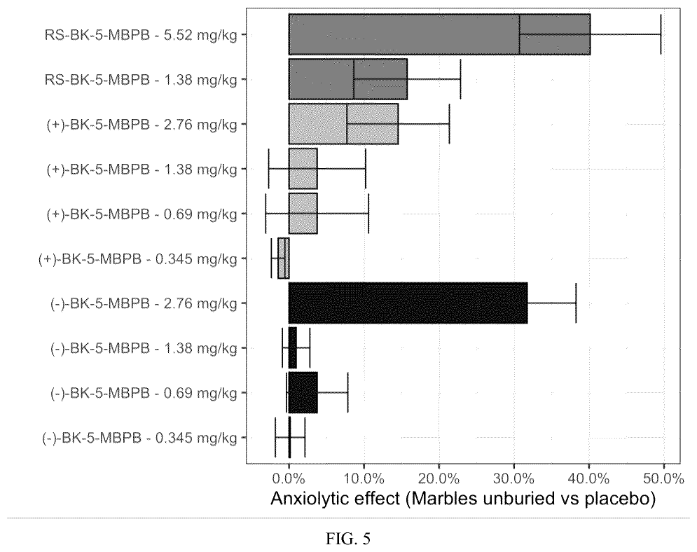

While 0.6 mg/kg of either enantiomer was ineffective, clear effects were seen when the two enantiomers were given simultaneously as 1.2 mg/kg of the racemate, illustrated in FIG. 5. In contrast, other compounds appeared to have roughly linear interactions where the effects of the racemate appeared to be adequately approximated by the sum of the effects of the individual enantiomers.

Based on this finding of potential non-additive effects, further experiments were conducted in which the ratio of 5-MAPB enantiomers was varied. Including the previous results, this resulted in the following dose combinations:
0 mg/kg S-enantiomer+0 mg/kg R-enantiomer;
0.3 mg/kg S-enantiomer+0 mg/kg R-enantiomer;
0.6 mg/kg S-enantiomer+0 mg/kg R-enantiomer;
1.2 mg/kg S-enantiomer+0 mg/kg R-enantiomer;
0 mg/kg S-enantiomer+0.3 mg/kg R-enantiomer;
0 mg/kg S-enantiomer+0.6 mg/kg R-enantiomer;
0 mg/kg S-enantiomer+1.2 mg/kg R-enantiomer;
0.6 mg/kg S-enantiomer+0.15 mg/kg R-enantiomer;
0.6 mg/kg S-enantiomer+0.3 mg/kg R-enantiomer;
0.6 mg/kg S-enantiomer+0.45 mg/kg R-enantiomer; and
0.6 mg/kg S-enantiomer+0.6 mg/kg R-enantiomer.

The resulting data were analyzed with a linear model in which marble burying was predicted by S-dose, R-dose and an interaction term. The overall model was significant (F-statistic: 20.3 on 3 and 216 DF, p-value: <0.001, adjusted R²: 0.2091) and there were significant effects of S-dose (T-value −4.382, p<0.001), R-dose (T-value −2.388, p=0.018), and the interaction term (T-value −2.073, p=0.039). This confirmed a surprising interactive effect when both enantiomers were given simultaneously that was not explainable by dose of either enantiomer alone.

### Marble Burying Experimental Methods

Marble burying experiments were conducted by trained and authorized personnel and were in compliance with applicable guidelines for experiments with laboratory animals. Manipulation of animals was conducted carefully to reduce stress to a minimum.

### Animal Care

Test animals are Swiss CD1 mice, 5-6 weeks old, that have not been subjected to prior experiments.

### Housing Conditions

| | |
|---|---|
| Housing | Group housing (8-9 mice/cage): 1290D Eurostandard |
| | Type III cages (Tecniplast, Italy) in transparent |
| | polycarbonate (42.5 cm deep; 26.6 cm large; 15.5 |
| | cm high, area = 820 cm²). |
| | Cages are covered with a stainless steel grid in |
| | which food and a bottle are placed. A stainless |
| | steel removable divider separates food and water |
| Litter | Aspen Small (SDS Dietex, France) |
| Enrichment | Cell huts |
| Temperature | 21.5 ± 1.5° C. |
| Hygrometry | 50 ± 30% (measured but not controlled) |
| Air renewal | Fresh air, 12-25 vol/h |
| Lighting | 20-30 Lux |
| Day/night | Normal 12 h/12 h cycle; light on 8:00-20:00/off: |
| cycle | 20:00-8:00 |
| Food | Rat-mouse A04 (Safe, France) available ad libitum |
| Drink | Tap water, available ad libitum |

### Experimental Arenas

The experiment was conducted in eight Plexiglas transparent open boxes (42 cm L, 42 cm W, 40 cm H) filled with 5 cm sawdust. Twenty-five clean glass marbles (15 mm diameter) were evenly spaced 5 cm apart on sawdust.

### Testing Procedure

Testing was carried out during the dark phase, in standardized conditions (T°=22.0±1.5° C.), with artificial light (20 Lux at the level of the apparatus) and low ambient noise (mostly coming from the ventilation system and the experimental apparatus).

Test compounds or placebo vehicle were administered intraperitoneally 30 minutes before animals were individually placed in an experimental apparatus for a 30-min session.

The number of marbles at least ⅔ buried was counted at the end of the session as the primary outcome measure. Results were generally displayed with scores inverted (proportion of marble left unburied) and expressed as magnitude difference-from-placebo with error bars indicating 95% confidence intervals.

### EXAMPLE 6: In Vitro Binding Site Studies

Select compounds of the present invention were tested for agonist and antagonist activity against 5-HT_{1B} and 5-HT_{2A} and the results are shown in Table 1. Select compounds were also tested for adrenergic 32 receptor antagonist activity, MAO-A inhibition, and the ability to inhibit nicotinic acetylcholine α4/β2 receptors. The results are shown in Table 2.

Adrenergic P2 Receptor cAMP Secondary Messenger Antagonist Assay Methods

This assay used a panel of CHO-K1 cell lines stably expressing non-tagged GPCRs that endogenously signal through cAMP. Hit Hunter® cAMP assays monitored the activation of a GPCR via Gi and Gs secondary messenger signaling in a homogenous, non-imaging assay format using DiscoverX Enzyme Fragment Complementation (EFC) with β-galactosidase as the functional endpoint.

The enzyme was split into two complementary portions: Enzyme Acceptor (EA) and Enzyme Donor (ED). In the assay, exogenously introduced ED fused to cAMP (ED-cAMP) competed with endogenously generated cAMP for binding to an anti-cAMP-specific antibody. Active β-galactosidase was formed by complementation of exogenous EA to any unbound ED-cAMP. Active enzyme could then convert a chemiluminescent substrate, generating an output signal detectable on a standard microplate reader.

Cell lines were expanded from freezer stocks according to standard procedures. Cells were seeded in a total volume of 20 μL into white walled, 384-well microplates and incubated at 37° C. for the appropriate time prior to testing. cAMP modulation was determined using the DiscoverX HitHunter cAMP XS+ assay.

Test compounds were assayed at 10 concentrations with the highest concentration either 30 or 10 μM and subsequent concentrations using a 0.33 dilution factor.

For agonist determination, cells were incubated with sample (in the presence of EC80 forskolin to induce response if measuring Gi secondary messenger signaling). Media was aspirated from cells and replaced with 15 μL 2:1 HBSS/10 mM Hepes: cAMP XS+Ab reagent. Intermediate dilution of sample stocks was performed to generate 4× sample in assay buffer (optionally containing 4× EC80 forskolin). 5 μL of 4× sample was added to cells and incubated at 37° C. or room temperature for 30 or 60 minutes, as appropriate. Final assay vehicle concentration was 1%.

For antagonist determination, cells were pre-incubated with sample followed by agonist challenge at the EC80 concentration. Media was aspirated from cells and replaced with 10 μL 1:1 HBSS/Hepes: cAMP XS+Ab reagent. 5 μL of 4× compound was added to the cells and incubated at 37° C. or room temperature for 30 minutes. 5 μL of 4× EC80 agonist was added to cells and incubated at 37-C or room temperature for 30 or 60 minutes. For Gi coupled GPCRs, EC80 forskolin was included.

After appropriate compound incubation, assay signal was generated through incubation with 20 μL cAMP XS+ED/CL lysis cocktail for one hour followed by incubation with 20 μL cAMP XS+EA reagent for three hours at room temperature. Microplates were read following signal generation with a PerkinElmer Envision™ instrument for chemiluminescent signal detection.

Compound activity was analyzed using CBIS data analysis suite (ChemInnovation, CA). For Gs antagonist mode assays, percentage inhibition was calculated as 100%×(1−(mean RLU of test sample−mean RLU of vehicle control)/(mean RLU of EC80 control−mean RLU of vehicle control)).

5-HT₂A and 5-HT₂B Agonist and Antagonist Assays

The DiscoveRx Calcium NWPLUS Assay was used for detection of changes in intracellular calcium as signaled by an increase of dye fluorescence in cells expressing 5-HT₂A receptors. Signal was measured on a fluorescent plate reader equipped with fluidic handling capable of detecting rapid changes in fluorescence upon compound stimulation.

To conduct the assay, cell lines were expanded from freezer stocks according to standard procedures. Cells (10,000cells/well) were seeded in a total volume of 50 μL (200 cells/L) into black-walled, clear-bottom, Poly-D-lysine coated 384-well microplates and incubated at 37-C for the appropriate time prior to testing. DMSO concentration for all readouts was <0.2%.

Assays were performed in 1× DyeLoading Buffer consisting of 1× Dye (DiscoverX, Calcium No WashPLUS kit, Catalog No. 90-0091), 1× Additive A and 2.5 mM Probenecid in HBSS/20 mM Hepes. Probenecid was prepared fresh. Cells were loaded with dye prior to testing. Media was aspirated from cells and replaced with 25 μL Dye Loading Buffer. Test compounds were assayed at 10 concentrations with the highest concentration either 30 or 10 μM and subsequent concentrations using a 0.33 dilution factor. Cells with testing sample were incubated for 45 minutes at 37° C. and then 20 minutes at room temperature. After dye loading, cells were removed from the incubator and 25 μL of 2×compound in HBSS/20 mM Hepes was added using a FLIPR Tetra (MDS). For 5-HT₂A assays, serotonin and altanserin were used as agonist and antagonist reference controls. For 5-HT₂B assays, these were serotonin and LY272015.

For antagonist determination, cells were pre-incubated with sample followed by agonist challenge at the EC₈₀ concentration. After dye loading, cells were removed from the incubator and L 2× sample was added. Cells were incubated for 30 minutes at room temperature in the dark to equilibrate plate temperature. After incubation, antagonist determination was initiated with addition of 25 μL 1× compound with 3× EC₈₀ agonist using FLIPR.

Compound agonist activity was measured on a FLIPR Tetra. Calcium mobilization was monitored for 2 minutes with a 5 second baseline read. FLIPR read-Area under the curve was calculated for the two minute read. Compound activity was analyzed using CBIS data analysis suite (ChemInnovation, CA). Percentage activity was calculated as 100%×(mean RFU of test sample−mean RFU of vehicle control)/(mean MAX RFU control ligand −mean RFU of vehicle control). For antagonist mode assays, percentage inhibition was calculated as 100%×(1−(mean RFU of test sample−mean RFU of vehicle control)/(mean RFU of EC₈₀ control−mean RFU of vehicle control)).

### MAO-A Inhibition Assay

MAO-A and test compounds were preincubated at 37° C. for 15 minutes before substrate addition. Test compounds were assayed at 10 concentrations with the highest concentration either 30 or 10 μM and subsequent concentrations using a 0.33 dilution factor. The reaction was initiated by addition of kynuramine and incubated at 37° C. for 30 minutes. The reaction was terminated by addition of NaOH. The amount of 4-hydroquioline formed was determined through spectrofluorimetric readout with the emission detection at 380 nm and excitation wavelength 310 nm. Clorgyline (IC₅₀ 0.00438 μM) was used as a positive control.

Nicotinic Acetylcholine Receptor α4β2 (nAchRa4/b2) Ion Channel Blocking Assay

Cell lines were expanded from freezer stocks according to standard procedures. Cells were seeded in a total volume of 20 μL into black-walled, clear-bottom, Poly-D-lysine coated 384-well microplates and incubated at 37-C for the appropriate time prior to testing.

Assays were performed in IX Dye Loading Buffer consisting of IX Dye, and 2.5 mM freshly-prepared Probenecid when applicable.

Test compounds were assayed at 10 concentrations with the highest concentration either 30 or 10 μM and subsequent concentrations using a 0.33 dilution factor.

Prior to testing, cells were loaded with dye then incubated for 30-60 minutes at 37° C. For antagonist determination, cells were pre-incubated with sample. Dihydro-β-erythroidine was used as a positive control. Intermediate dilution of sample stocks was performed to generate 2-5× sample in assay buffer.

After dye loading, cells were moved from the incubator and 10-25 μL 2-5× sample was added to cells in the presence of EC80 agonist when appropriate. Cells were incubated for 30 minutes at room temperature in the dark to equilibrate plate temperature. Vehicle concentration was 100.

Compound activity was measured on a FLIPRTetra(MIDS) and analyzed using CBIS data analysis suite (ChemInnovation, CA). Percentage inhibition was calculated using the following formula: % Inhibition=100%×(1−(mean RLU of test sample−mean RLU of vehicle control)/(mean RLU of EC80 control−mean RLU of vehicle control)).

TABLE 1
Agonist and Antagonist activity against 5-HT_{1B} and 5-HT_{2A}

| | 5HT_{1B} | 5HT_{2A} | 5HT_{2A} | 5HT_{2B} | 5HT_{2B} |
|---|---|---|---|---|---|
| | Agonist | Agonist | Antagonist | Agonist | Antagonist |
| Compound | (μM) | (μM) | (μM) | (μM) | (μM) |
| 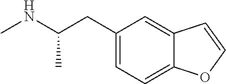 S-5-MAPB [1-(1-benzofuran-5- yl)propan-2- yl](methyl)amine | 0.16 | ND | ND | ND | ND |
| 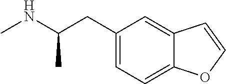 R-5-MAPB [1-(1-benzofuran-5- yl)propan-2- yl](methyl)amine | 0.98 | 2.40 | 5.34 | ND | 0.36 |
| 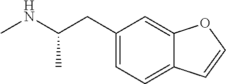 S-6-MAPB [1-(1-benzofuran-6- y1)propan-2- yl](methyl)amine | 0.10 | ND | ND | ND | ND |
| 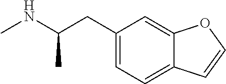 R-6-MAPB [1-(1-benzofuran-6- yl)propan-2- yl](methyl)amine | 1.48 | 4.62 | ND | ND | 0.40 |
| 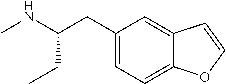 S-5-MBPB [1-(1-benzofuran-5- yl)butan-2- yl](methyl)amine | 3.06 | ND | ND | ND | ND |
| 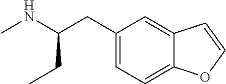 R-5-MBPB [1-(1-benzofuran-5- yl)butan-2- yl](methyl)amine | ND | ND | ND | ND | ND |
| 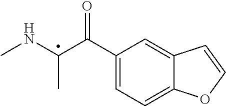 Enantiomer 1 BK-5-MAPB 1-(1-benzofuran-5-yl)-2- (methylamino)propan-1- one | 0.08 | ND | ND | ND | ND |
| 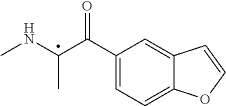 Enantiomer 2 BK-5-MAPB 1-(1-benzofuran-5-yl)-2- (methylamino)propan-1- one | 3.07 | ND | ND | ND | ND |

TABLE 2
Adrenergic β ₂ receptor antagonist activity, MAO-A inhibition, and nicotinic
acetylcholine α4/β2 receptor blocking activity of select compounds

| | ADREN R β 2 | MAO-A Inhibitor | nAChR(α4/β2) |
|---|---|---|---|
| Compound | Antagonist (μM) | (μM) | Blocker (μM) |
| 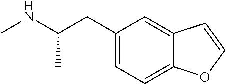 S-5-MAPB [1-(1-benzofuran-5- yl)propan-2- yl](methyl)amine | ND | 1.29 | ND |
| 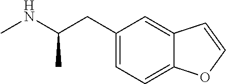 R-5-MAPB [1-(1-benzofuran-5- yl)propan-2- yl](methyl)amine | ND | 3.94 | 4.24 |
| 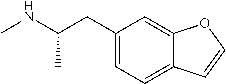 S-6-MAPB [1-(1-benzofuran-6- yl)propan-2- yl](methyl)amine | ND | 5.39 | ND |
| 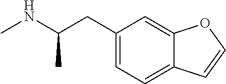 R-6-MAPB [1-(1-benzofuran-6- yl)propan-2- yl](methyl)amine | 4.06 | 2.12 | 7.41 |
| 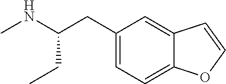 S-5-MBPB [1-(1-benzofuran-5- yl)butan-2- yl](methyl)amine | ND | 2.66 | 9.98 |
| 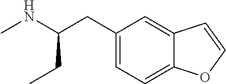 R-5-MBPB [1-(1-benzofuran-5- yl)butan-2- yl](methyl)amine | ND | ND | 6.33 |

### EXAMPLE 7: 5-HT1BR cAMP Secondary Messenger Agonist Assay

The 5-HT_{1B}R cAMP secondary messenger agonist assay used a panel of CHO-K1 cell lines stably expressing non-tagged GPCRs that endogenously signal through cAMP. Hit Hunter® cAMP assays monitored the activation of a GPCR via Gi and Gs secondary messenger signaling in a homogenous, non-imaging assay format using DiscoverX Enzyme Fragment Complementation (EFC) with β-galactosidase as the functional endpoint.

The enzyme was split into two complementary portions: Enzyme Acceptor (EA) and Enzyme Donor (ED). Exogenously introduced ED fused to cAMP (ED-cAMP) competed with endogenously generated cAMP for binding to an anti-cAMP-specific antibody. Active β-galactosidase was formed by complementation of exogenous EA to any unbound ED-cAMP. Active enzyme could then convert a chemiluminescent substrate, generating an output signal detectable on a standard microplate reader.

Cell lines were expanded from freezer stocks according to standard procedures. Cells were seeded in a total volume of 20 μL into white walled, 384-well microplates and incubated at 37-C for the appropriate time prior to testing. cAMP modulation was determined using the DiscoverX HitHunter cAMP XS+ assay.

For agonist determination, cells were incubated with sample (in the presence of EC₈₀ forskolin to induce response if measuring Gi secondary messenger signaling). Media was aspirated from cells and replaced with 15 μL 2:1 HBSS/10 mM Hepes: cAMP XS+Ab reagent. Intermediate dilution of sample stocks was performed to generate 4× sample in assay buffer (optionally containing 4× EC₈₀ forskolin). 5 μL of 4× sample was added to cells and incubated at 37-C or room temperature for 30 or 60 minutes, as appropriate. Final assay vehicle concentration was 1%. The results are shown in Table 3.

In certain embodiments a benzofuran derivatives of the current invention are 5-HT1BR agonists. Direct stimulation of 5-HT1BR has not been previously documented with drugs producing MDMA-like effects and MDMA itself does not bind to the 5-HT1BR (Ray. 2010. PloS one, 5(2), e9019). Indirect stimulation of 5-HT1BR, secondary to elevated extracellular serotonin, has been hypothesized to be required for the prosocial effects of MDMA (Heifets et al. 2019. Science translational medicine, 11(522)), while other aspects of entactogen effects have been attributed to monoamine release (e.g., Luethi & Liechti. 2020. Archives of toxicology, 94(4), 1085-1133).

Thus, in one embodiment, the unique ratios of 5-HT1BR stimulation and monoamine release displayed by the disclosed compounds enable different profiles of therapeutic effects that cannot be achieved by MIDMA or other known entactogens.

TABLE 3
5-HT1B Agonist Effects of N-alkyl Benzofuran Compounds

| | 5-HT1BR | |
|---|---|---|
| Compound | EC50 (μM) | Hill Coef. |
| 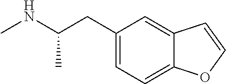 S-5-MAPB | 0.16147 | 1.08 |
| 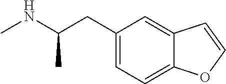 R-5-MAPB | 0.98089 | 1.13 |
| 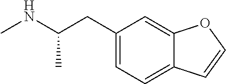 S-6-MAPB | 0.10166 | 1.21 |
| 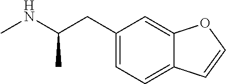 R-6-MAPB | 1.48187 | 1.14 |
| 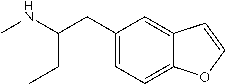 rac-5-MBPB | 5.82237 | 0.96 |
| 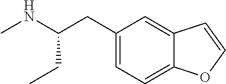 S-5-MBPB | 3.0638 | 1.5 |
| 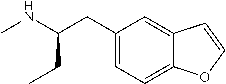 R-5-MBPB | >10 | |
| 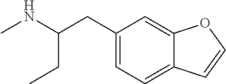 rac-6-MBPB | 1.95124 | 0.89 |
| 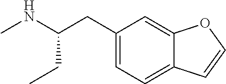 S-6-MBPB | 1.72234 | 1.1131 |
| 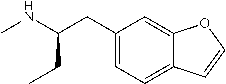 R-6-MBPB | 5.75045 | 0.8121 |
| 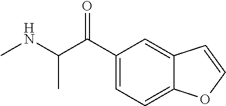 rac-BK-5-MAPB | 0.13951 | 0.91 |
| 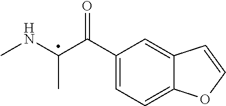 (−)-BK-5-MAPB | 0.0777 | 1.5 |
| 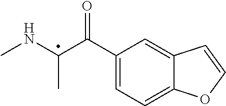 (+)-BK-5-MAPB | 3.06782 | 1.05 |
| 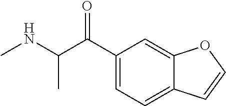 rac-BK-6-MAPB | 0.5373 | 0.99 |
| 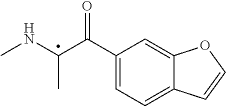 (−)-BK-6-MAPB | 0.28804 | 0.8339 |
| 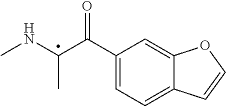 (+)-BK-6-MAPB | >10 | |
| 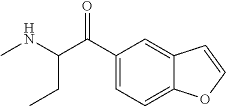 [SMILES: CCC(NC)C(=O)c1ccc2occc2c1] rac-BK-5-MBPB | 19.64355 | 1.01 |
| 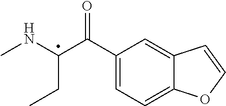 [SMILES: CCC(NC)C(=O)c1ccc2occc2c1] (−)-BK-5-MBPB | 6.61190 | 1.6241 |
| 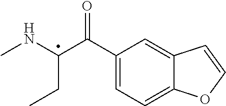 [SMILES: CCC(NC)C(=O)c1ccc2occc2c1] (+)-BK-5-MBPB | >30 | |
| 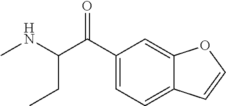 [SMILES: CCC(NC)C(=O)c1ccc2ccoc2c1] rac-BK-6-MBPB | >30 | |
| 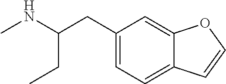 (−)-BK-6-MBPB | 7.51594 | 1.9551 |
| 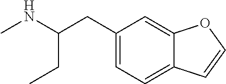 (+)-BK-6-MBPB | >30 | |

### EXAMPLE 8: Human Serotonin Transporter (SERT, SLC6A4) Functional Antagonist Uptake Assay

Benzofuran derivatives were evaluated for inhibiting the human 5-HT transporter (hSERT) as expressed in CHO cells using an antagonist radioligand assay (Tatsumi, M. et al. (1999), Eur. J. Pharmacol., 368: 277-283). Compound binding was calculated as a percent inhibition of the binding of 2 nM [³H]imipramine using a scintillation method and inhibition constants (Ki) were calculated using the Cheng Prusoff equation. Test compounds were assayed in three trials at 300, 94.868, 30, 9.4868, 0.3, and 0.94868 μM.

All tested compounds showed inhibition of hSERT at the tested concentrations. However, in two cases (the enantiomers of 5-MBPB), the lowest concentration of 0.94868 μM was too high to accurately estimate IC₅₀ values and Ki values. For S-(+)-5-MBPB the IC₅₀ appeared close to 0.094868 μM, while for R-(−)-5-MBPB the IC₅₀ appeared close to 0.94868 μM.

When compounds are substrates for monoamine transporters instead of solely inhibitors, it is known that IC₅₀ values underestimate their potency for interacting with these transporters (Ilic, M. et al. (2020), Frontiers in Pharmacology 11: 673).

TABLE 4
Human Serotonin Transporter Functional Antagonist Uptake Assay

| Compound | SERT IC50 (μM) | SERT Ki (μM) |
|---|---|---|
| 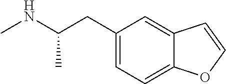 S-(+)-5-MAPB | 3.40 | 1.50 |
| 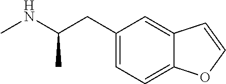 R-(−)-5-MAPB | 2.70 | 1.20 |
| 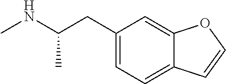 S-(+)-6-MAPB | 6.70 | 3.10 |
| 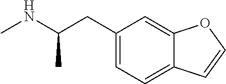 R-(−)-6-MAPB | 24.0 | 11.0 |
| 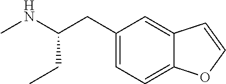 S-(+)-5-MBPB | <0.95 | ND |
| 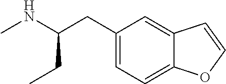 R-(−)-5-MBPB | <0.95 | ND |
| 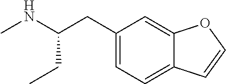 S-(+)-6-MBPB | 2.70 | 1.20 |
| 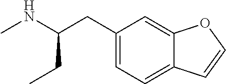 R-(−)-6-MBPB | 5.70 | 2.60 |
| 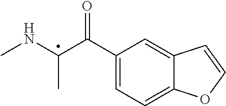 (−)-BK-5MAPB | 6.90 | 3.20 |
| 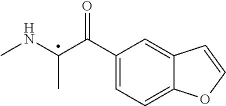 (+)-BK-5MAPB | 38.0 | 17.0 |
| 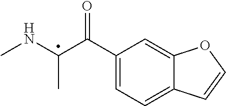 (−)-BK-6MAPB | 12.0 | 5.40 |
| 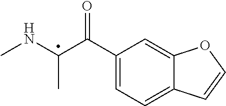 (+)-BK-6MAPB | 110 | 51.0 |
| 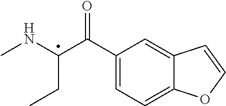 [SMILES: CCC(NC)C(=O)c1ccc2occc2c1] (−)-BK-5MBPB | 1.60 | 0.720 |
| 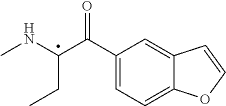 [SMILES: CCC(NC)C(=O)c1ccc2occc2c1] (+)-BK-5MBPB | 6.50 | 3.00 |
| 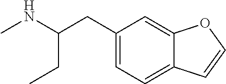 (−)-BK-6MBPB | 3.70 | 1.70 |
| 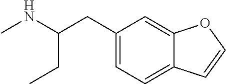 (+)-BK-6MBPB | 29.0 | 13.0 |

### EXAMPLE 9: Effects of Substituted Benzofurans on Extracellular Serotonin

Select compounds of the present invention were studied for their effect on extracellular serotonin and compared to MDMA. The results are shown in Table 5.

TABLE 5
Effects of Substituted Benzofurans on Extracellular Serotonin

| | Inhibition of | |
|---|---|---|
| | [3H]5-HT | [3H]5-HT |
| | Uptake at | release via |
| | SERT | SERT |
| Compound | IC₅₀ (nM) | EC₅₀ (nM) |
| 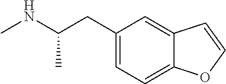 S-5-MAPB | 60.4 ± 2.8 | 13.0 ± 1.2 |
| 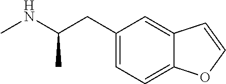 R-5-MAPB | 149.6 ± 8.6 | 29.3 ± 3.7 |
| 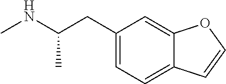 S-6-MAPB | 90.9 ± 5.9 | 20.6 ± 2.7 |
| 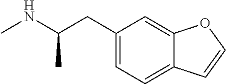 R-6-MAPB | 622.6 ± 31.2 | 111.9 ± 17.5 |
| 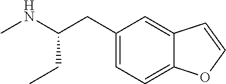 S-5-MBPB | 123.7 ± 35.8 | 31.2 ± 14.3 |
| 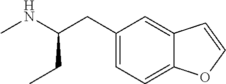 R-5-MBPB | 211.7 ± 36.2 | 49.5 ± 27.1 |
| 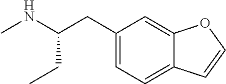 S-6-MBPB | 216.8 ± 45.8 | 54.1 ± 33.9 |
| 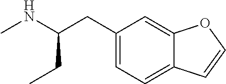 R-6-MBPB | 702.5 ± 261.7 | 171.7 ± 82.2 |
| 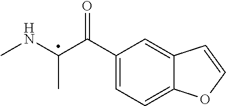 (−)-BK-5-MAPB | 284.4 ± 11.9 | 80.0 ± 13.7 |
| 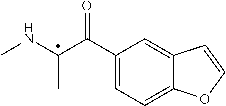 (+)-BK-5-MAPB | 2087.0 ± 151.0 | 438.8 ± 72.5 |
| 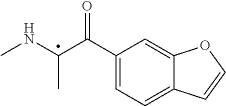 (−)-BK-6-MAPB | 274.6 ± 13.0 | 72.2 ± 16.6 |
| 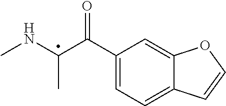 (+)-BK-6-MAPB | 5466.0 ± 424.0 | 1283.0 ± 268.0 |
| 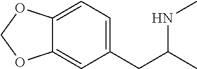 MDMA | 384.5 ± 32.8 | 94.3 ± 13.6 |

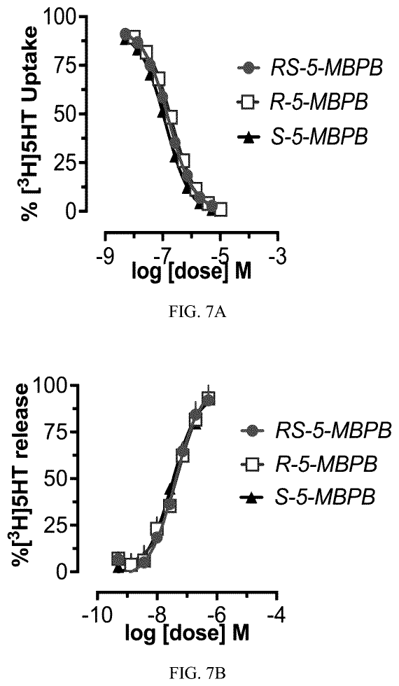

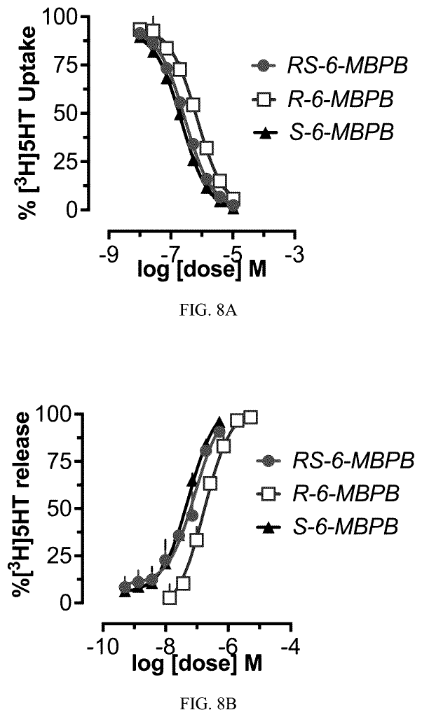

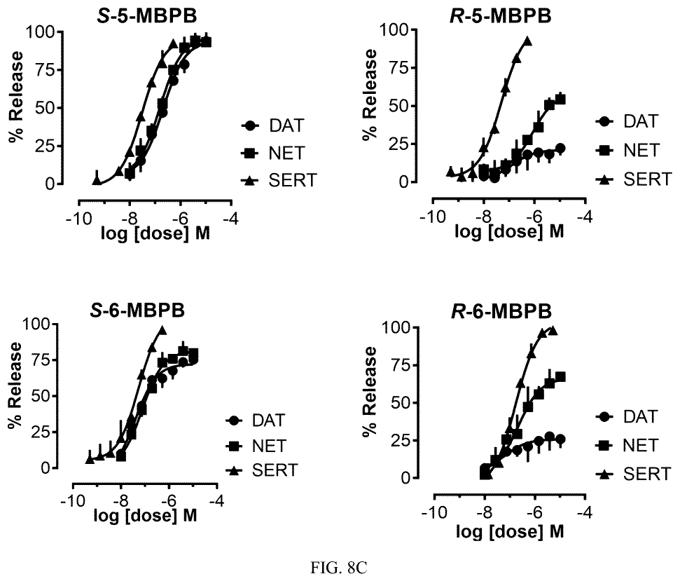

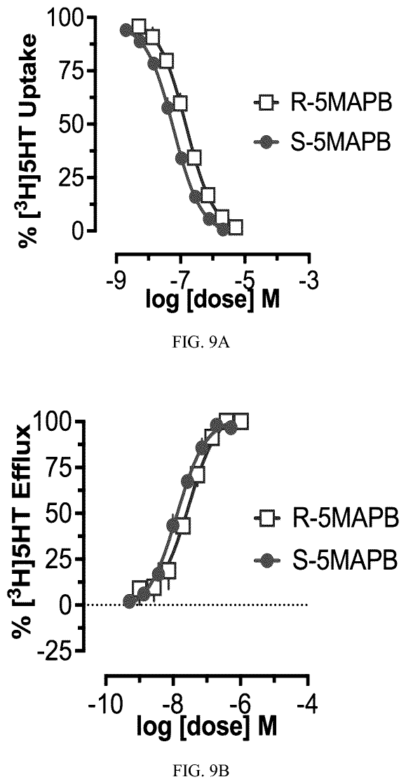

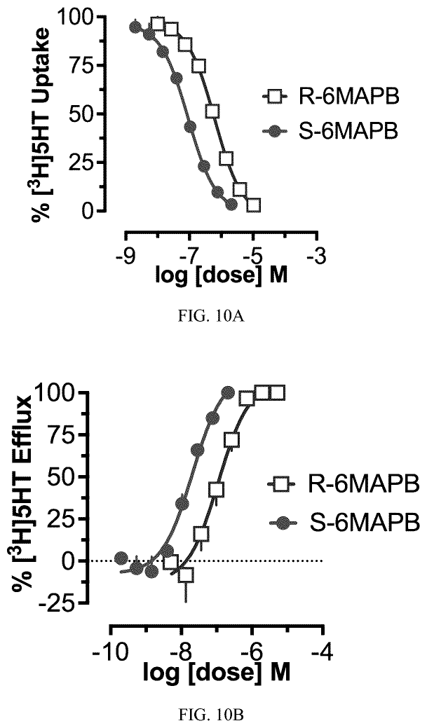

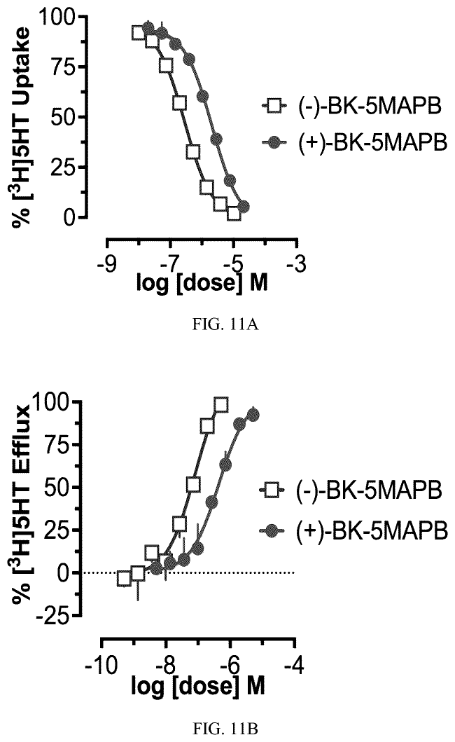

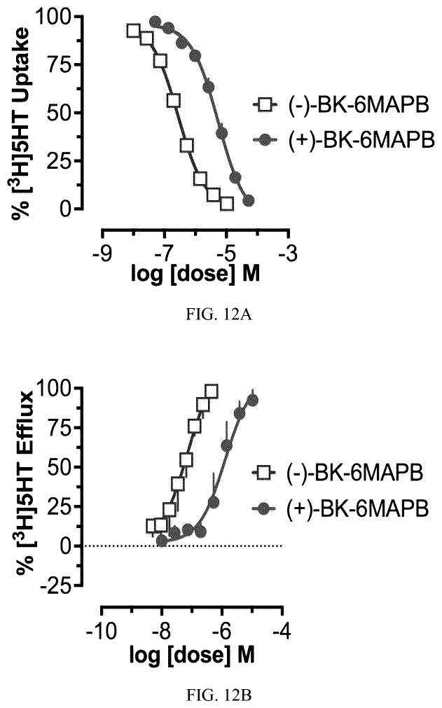

The compounds were efficacious at rapidly increasing extracellular serotonin, which produces rapid therapeutic effects. FIG. 7A-FIG. 12B show in vitro rat synaptosome assay results that demonstrate serotonin reuptake inhibition and release of the compounds in Table 5. FIG. 7A is a graph of the effect of RS-5-MBPB, R-5-MBPB, and S-5-MBPB on 5HT uptake and FIG. 7B is a graph of the effect of RS-5-MBPB, R-5-MBPB, and S-5-MBPB on 5HT release. FIG. 8A is a graph of the effect of RS-6-MBPB, R-6-MBPB, and S-6-MBPB on 5HT uptake and FIG. 8B is a graph of the effect of RS-6-MBPB, R-6-MBPB, and S-6-MBPB on 5HT release. FIG. 9A and FIG. 9B are graphs of the effect of R-5-MAPB and S-5-MAPB on serotonin uptake and release, respectively. FIG. 10A and FIG. 10B are graphs of the effect of R-6-MAPB and S-6-MAPB on serotonin uptake and release, respectively. FIG. 11A and FIG. 11B are graphs of the effect of (−)-Bk-5-MAPB and (+)-Bk-5-MAPB on serotonin uptake and release, respectively. FIG. 12A and FIG. 12B are graphs of the effect of (−)-Bk-6-MAPB and (+)-Bk-6-MAPB on serotonin uptake and release, respectively.

Male Sprague-Dawley rats (Charles River, Kingston, NY, USA) were used for the synaptosome assays. Rats were group-housed with free access to food and water, under a 12 hour light/dark cycle with lights on at 0700 hours. Rats were euthanized by CO₂ narcosis, and synaptosomes were prepared from brains using standard procedures (Rothman, R. B., & Baumann, M. H. (2003) Monoamine transporters and psychostimulant drugs. European journal of pharmacology, 479(1-3), 23-40) Transporter uptake and release assays were performed as described previously (Solis et al. (2017). N-Alkylated analogs of 4-methylamphetamine (4-MA) differentially affect monoamine transporters and abuse liability. Neuropsychopharmacology, 42(10), 1950-1961). In brief, synaptosomes were prepared from whole brain minus caudate and cerebellum for serotonin (5-HT) transporter (SERT) assays.

For the SERT uptake inhibition assay, 5 nM [3H]5-HT was used. To optimize uptake for a single transporter, unlabeled blockers were included to prevent the uptake of [3H]5-HT by competing transporters. Uptake inhibition was initiated by incubating synaptosomes with various doses of test compound and [3H]5-HT in Krebs-phosphate buffer. Uptake assay was terminated by rapid vacuum filtration and retained radioactivity was quantified with liquid scintillation counting (Baumann et al. (2013) Powerful cocaine-like actions of 3, 4-methylenedioxypyrovalerone (MDPV), a principal constituent of psychoactive ‘bath salts’ products. Neuropsychopharmacology, 38(4), 552-562). Results of the experiment are shown in FIG. 7A (for RS-5-MBPB, R-5-MBPB, and S-5-MBPB), FIG. 8A (for RS-6-MBPB, R-6-MBPB, and S-6-MBPB), FIG. 9A (for R-5-MAPB and S-5-MAPB), FIG. 10A (for R-6-MAPB and S-6-MAPB), FIG. 11A (for (−)-Bk-5-MAPB and (+)-Bk-5-MAPB), and FIG. 12A (for (−)-Bk-6-MAPB and (+)-Bk-6-MAPB).

For the release assay, 5 nM [3H]5-HT was used for SERT. All buffers used in the release assay contained 1 μM reserpine to block vesicular uptake of substrates. The selectivity of the release assay was optimized for a single transporter by including unlabeled blockers to prevent the uptake of [3H]5-HT by competing transporters. Synaptosomes were preloaded with radiolabeled substrate in Krebs-phosphate buffer for 1 hour to reach steady state. The release assay was initiated by incubating preloaded synaptosomes with various concentrations of the test drug. Release was terminated by vacuum filtration and retained radioactivity quantified by liquid scintillation counting. Results of the experiment are shown in FIG. 7B (for RS-5-MBPB, R-5-MBPB, and S-5-MBPB), FIG. 8B (for RS-6-MBPB, R-6-MBPB, and S-6-MBPB), FIG. 9B (for R-5-MAPB and S-5-MAPB), FIG. 10B (for R-6-MAPB and S-6-MAPB), FIG. 11B (for (−)-Bk-5-MAPB and (+)-Bk-5-MAPB), and FIG. 12B (for (−)-Bk-6-MAPB and (+)-Bk-6-MAPB).

Effects of test drugs on release were expressed as a percent of maximal release, with maximal release (i.e., 100% Eₘₐₓ) defined as the release produced by tyramine at doses that evoked the efflux of all ‘releasable’ tritium by synaptosomes (100 μM tyramine for SERT assay conditions). Effects of test drugs on uptake inhibition and release were analyzed by nonlinear regression. Dose-response values for the uptake inhibition and release were fit to the equation, Y(x)=Yₘᵢₙ+(Yₘₐₓ−Yₘᵢₙ)/(1+10exp[(log P₅₀−log x)]×n), where x was the concentration of the compound tested, Y(x) was the response measured, Yₘₐₓ was the maximal response, P₅₀ was either IC₅₀ (the concentration that yielded half-maximal uptake inhibition response) or EC₅₀ (the concentration that yielded half-maximal release), and n was the Hill slope parameter.

Similarly, caudate tissue can be used for dopamine transporter (DAT) and whole brain minus caudate and cerebellum can be used for norepinephrine transporter (NET) assays. For these uptake inhibition assays, 5 nM [3H]dopamine or [3H]norepinephrine can be used for DAT or NET assays respectively. To optimize uptake for a single transporter, unlabeled blockers are included to prevent the uptake of [3H]transmitter by competing transporters. Uptake inhibition is initiated by incubating synaptosomes with various doses of test compound and [3H]transmitter in Krebs-phosphate buffer. Uptake assays are terminated by rapid vacuum filtration and retained radioactivity is quantified with liquid scintillation counting (Baumann et al. (2013). Powerful cocaine-like actions of 3, 4-methylenedioxypyrovalerone (MDPV), a principal constituent of psychoactive ‘bath salts’ products. Neuropsychopharmacology, 38(4), 552-562).

Alternatively, for similar release assays, 9 nM [3H]MPP+ is used as the radiolabeled substrate for DAT and NET. All buffers in the release assay contain 1 μM reserpine to block vesicular uptake of substrates. The selectivity of release assays is optimized for a single transporter by including unlabeled blockers to prevent the uptake of [3H]MPP+ by competing transporters. Synaptosomes are preloaded with radiolabeled substrate in Krebs-phosphate buffer for 1 h to reach steady state. Release assays are initiated by incubating preloaded synaptosomes with various concentrations of the test drug. Release is terminated by vacuum filtration and retained radioactivity quantified by liquid scintillation counting.

Using these methods, enantiomers of 5-MBPB and 6-MBPB were assessed for their ability to increase extracellular dopamine and norepinephrine. Results of the experiment are shown in FIG. 8C. While all enantiomers of 5-MBPB and 6-MBPB were efficacious at increasing dopamine and norepinephrine, the Emaxes (maximum effects) were surprisingly found to be less than 100% for S-6-MBPB, R-5-MBPB, and R-6-MBPB. Both R-5-MBPB and R-6-MBPB appeared to be dopamine uptake inhibitors, with maximum dopamine increases close to about 25% of that produced by the reference drug. In contrast, S-6-MBPB appeared to be a partial releaser of dopamine with the highest concentration indicating an Emax of 72%. Similarly, S-6-MBPB, R-5-MBPB, and R-6-MBPB all appeared to be partial releasers of norepinephrine, with Emaxes of 81%, 58%, and 66%, respectively.

### EXAMPLE 10: In Vitro Absorption Assay

The permeability of 10 μM RS-5-MAPB in Caco-2, MDCKII, and MDR1-MDCKII cell line assays was assessed (Table 6). Both the AB and BA directions with and without the addition of a Pgp-specific inhibitor (verapamil), a MRP1 inhibitor (MK571), and an ATP-binding cassette subfamily G member 2 (ABCG2/BCRP) inhibitor (KG143) were measured. Trials were repeated twice, and values averaged. Results support that RS-5-MAPB was well absorbed and suggest that it is actively transported through a mechanism that was inhibited by verapamil.

TABLE 6
In vitro Absorption Assay of RS-5-MAPB

| | Permeability | | |
|---|---|---|---|
| | (Papp, 10⁻⁶ cm/s) | | |
| Assay System & Conditions | A-B | B-A | B-A/A-B |
| MDCKII, pH 7.4/7.4 | 82.1 | 19.8 | 0.241 |
| MDR1-MDCKII, pH 7.4/7.4 | 68.7 | 20.5 | 0.298 |
| MDR1-MDCKII, pH 7.4/7.4 + | 42.8 | 30.4 | 0.710 |
| verapamil | | | |
| Caco-2, pH 7.4/7.4 | 81 | 29.1 | 0.359 |
| Caco-2, pH 7.4/7.4 + verapamil | 37.6 | 23 | 0.612 |
| Caco-2, pH 7.4/7.4 + KO143 | 75.3 | 14 | 0.186 |

### EXAMPLE 11. 5-MAPB Freebase Isolation/Liquid-Liquid Extraction

Liquid-Liquid Extraction (LLE) was used to isolate 5-MAPB freebase from 5-MAPB hydrochloride (5-MAPB HCl) using the conditions in Table 7. FIG. 13 is the XRPD pattern for 5-MAPB HCl. (for 50 mg of 5-MAPB HCl, 1 vol. solvent is equivalent to 50 μL). The below technique was used to generate the XRPD pattern of 5-MAPB freebase in FIG. 14.

TABLE 7
Liquid-Liquid Extraction of RS-5-MAPB Freebase
Liquid-Liquid Extraction Expts.

| Expt. | Counter | Solvent | |
|---|---|---|---|
| No. | ion | (Density) | Procedure/Comments |
| 5-MAPB | NaOH | EtOAc | 50 mg of 5-MAPB (HCl salt) + 10 |
| | | (0.902 | vols. solvent + NaOH stock soln. |
| | | g/mL) | in water (1.1:1 molar ratio) + |
| | | | additional water (10 vols. water total) |
| 5-MAPB | NaOH | DCM | 50 mg of 5-MAPB (HCl salt) + 10 |
| | | (1.33 | vols. solvent + NaOH stock soln. |
| | | g/mL) | in water (1.1:1 molar ratio) + |
| | | | additional water (10 vols. water total) |

### EXAMPLE 12. PXRD Diffractogram Procedure

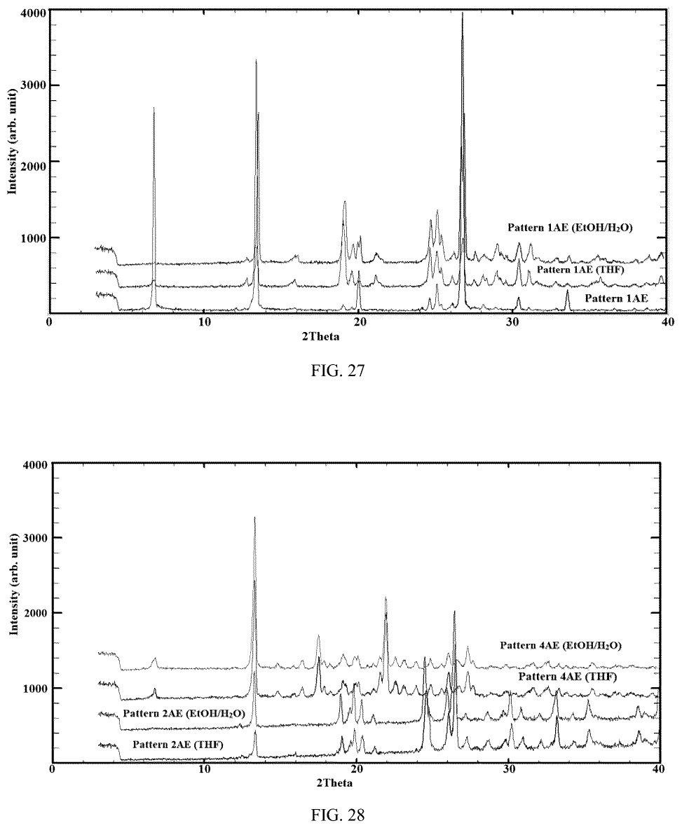

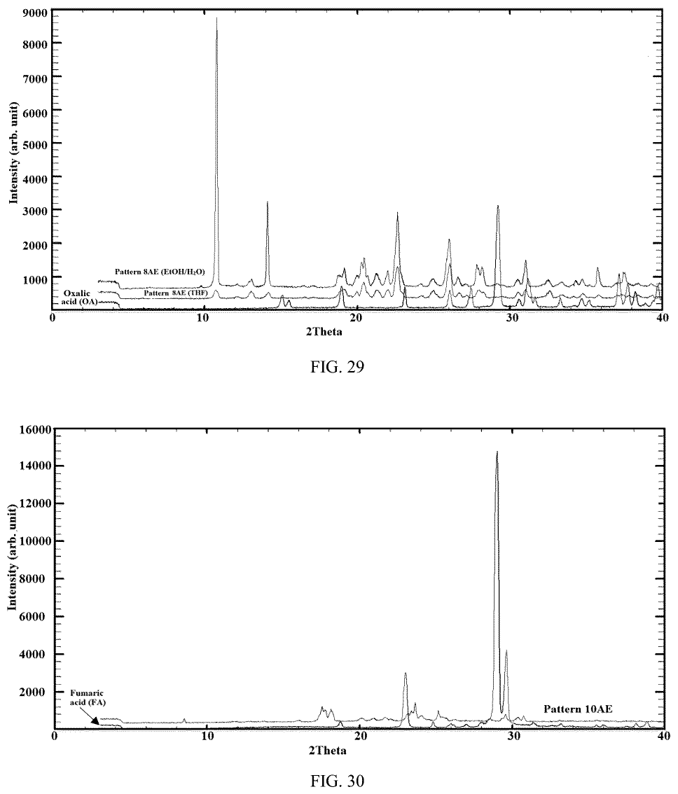

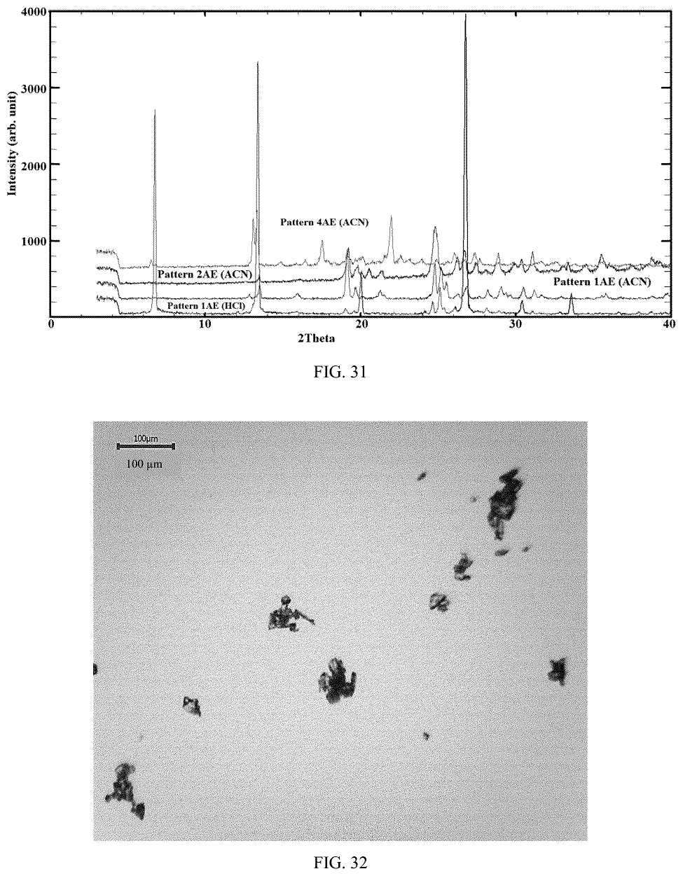

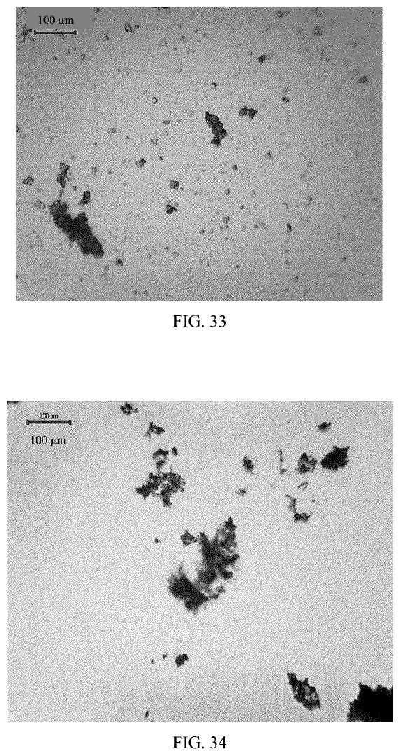

The PXRD diffractogram of RS-5-MAPB Pattern 1A, Pattern 2A, Pattern 4A, Pattern 4B, Pattern 4C and Pattern 10 were generated from RS-5-MAPB HCl. The below technique was used to generate the XRPD patterns in FIG. 13, FIG. 14, FIG. 15, FIG. 16, FIG. 17, FIG. 18, FIG. 19, FIG. 23, FIG. 24, FIG. 25, FIG. 26, FIG. 27, FIG. 28, FIG. 29, FIG. 30, and FIG. 31. Powder X-ray Diffraction (PXRD) patterns were collected on a Rigaku Miniflex Plus instrument. The instrument and method details are included below in the Table 8.

TABLE 8
PXRD diffractogram procedure

| | |
|---|---|
| Instrument: | Rigaku Miniflex Plus S/N ZD06186 |
| Sample Holder: | Aluminum Round Sample Holder with Zero |
| | Background Silicon Wafer |
| Scan Range: | 3° to 40° 20 |
| Scan Axis: | 20/0 |
| Scan Method: | Continuous |
| Sampling Width: | 0.02° |
| Scan Speed: | 0.62°/minute |
| Wavelength: | Cu Kα 1.54 Å |
| X-ray Generator: | 30 kV/15 mA |
| Detector: | Scintillation Counter |
| Scattering Slit: | 4.2° |
| Receiving Slit: | 0.3 mm |

### EXAMPLE 13. Salt Studies of 5-MAPB in Acetone or MeOH:1120

Salt studies of 5-MAPB were conducted using the conditions in Table 9. A total of 30 salt experiments for 15 counterions in 2 different solvents, acetone and MeOH:H₂O (90:10), were generated from RS-5-MAPB HCl. The below technique was used to generate the XRPD patterns in FIG. 15 and FIG. 16 (For 40 mg of 5-MAPB HCl, 1 vol. solvent=40 μL).

TABLE 9
Salt screening experiments

| | | | PXRD |
|---|---|---|---|
| Counterion | Solvent | Procedure/Comments | Result |
| HCl | Acetone | 40 mg 5-MAPB + 10 vols. solvent + 1:1 molar ratio | RS-5- |
| | | counterion soln., stirred at 40° C., became a slurry | MAPB |
| | | after ~30 min of stirring, in solution after overnight | Pattern |
| | | stirring, opened for evaporation, solid after ~3 hrs of | 1A |
| | | evaporation at 40° C., analyzed by PXRD, dried | |
| | | overnight at 40° C. in vacuum oven, analyzed by | |
| | | DSC and TGA | |
| HBr | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, solid | |
| | | after overnight evaporation, turned into gel during | |
| | | PXRD sample prep, terminated | |
| H₂SO₄ | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | overnight evaporation, terminated | |
| H₃PO₄ | Acetone | 40 mg 5-MAPB + 10 vols. solvent + 1:1 molar ratio | RS-5- |
| | | counterion soln., stirred at 40° C., became a slurry | MAPB |
| | | after ~30 min of stirring, slurry after overnight | Pattern |
| | | stirring, centrifuged to harvest solids, analyzed by | 4A |
| | | PXRD, dried overnight at 40° C. in vacuum oven, | |
| | | analyzed by DSC and TGA | |
| HNO₃ | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | overnight evaporation, terminated | |
| Methane | Acetone | 40 mg 5-MAPB + 10 vols. solvent + 1:1 molar ratio | N/A (gel) |
| Sulfonic | | counterion soln., stirred at 40° C., in solution after | |
| | | overnight stirring, opened for evaporation, gel after | |
| | | overnight evaporation, terminated | |
| Tartaric | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | 6 days evaporation, terminated | |
| Succinic | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | 6 days evaporation, terminated | |
| Oxalic | Acetone | 40 mg 5-MAPB + 10 vols. solvent + 1:1 molar ratio | RS-5- |
| | | counterion soln., stirred at 40° C., in solution after | MAPB |
| | | overnight stirring, opened for evaporation, solid | Pattern |
| | | after 6 days of evaporation, analyzed by PXRD, | 9A with |
| | | dried overnight at 40° C. in vacuum oven, analyzed | some |
| | | by DSC and TGA | Oxalic |
| | | | Peaks |
| Maleic | Acetone | 40 mg 5-MAPB + 10 vols. solvent + 1:1 molar ratio | RS-5- |
| | | counterion soln., stirred at 40° C., in solution after | MAPB |
| | | overnight stirring, opened for evaporation, solid | Pattern |
| | | after 6 days of evaporation, analyzed by PXRD, | 10A |
| | | dried overnight at 40° C. in vacuum oven, analyzed | |
| | | by DSC and TGA | |
| Malic | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | 6 days evaporation, terminated | |
| Citric | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | 6 days evaporation, terminated | |
| Fumaric | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | overnight evaporation, terminated | |
| Salicylic | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | overnight evaporation, terminated | |
| Hippuric | Acetone | 40 mg 5-MAPB + 10 vols. solvent + 1:1 molar ratio | N/A (gel) |
| | | counterion soln., stirred at 40° C., in solution after | |
| | | overnight stirring, opened for evaporation, gel after | |
| | | overnight evaporation, terminated | |
| HCl | MeOH:water | 40 mg 5-MAPB + 10 vols. solvent + 1:1 molar ratio | RS-5- |
| | 90:10 | counterion soln., stirred at 40° C., in solution after | MAPB |
| | | overnight stirring, opened for evaporation, solid | Pattern |
| | | after overnight evaporation, analyzed by PXRD | 1A |
| HBr | MeOH:water | 40 mg 5-MAPB + 10 vols. solvent + 1:1 molar ratio | RS-5- |
| | 90:10 | counterion soln., stirred at 40° C., in solution after | MAPB |
| | | overnight stirring, opened for evaporation, solid | Pattern |
| | | after overnight evaporation, analyzed by PXRD, | 2A |
| | | dried overnight at 40° C. in vacuum oven, analyzed | |
| | | by DSC and TGA | |
| H₂SO₄ | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | overnight evaporation, terminated | |
| H₃PO₄ | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | overnight evaporation, terminated | |
| HNO₃ | MeOH:water | 40 mg 5-MAPB + 10 vols. solvent + 1:1 molar ratio | N/A (gel) |
| | | 90:10 | |
| | | overnight stirring, opened for evaporation, gel after | |
| | | overnight evaporation, terminated | |
| Methane | MeOH:water | 40 mg 5-MAPB + 10 vols. solvent + 1:1 molar ratio | N/A (gel) |
| Sulfonic | 90:10 | counterion soln., stirred at 40° C., in solution after | |
| | | overnight stirring, opened for evaporation, gel after | |
| | | overnight evaporation, terminated | |
| Tartaric | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | 7 days of evaporation, terminated | |
| Succinic | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | 6 days evaporation, terminated | |
| Oxalic | MeOH:water | 40 mg 5-MAPB + 10 vols. solvent + 1:1 molar ratio | RS-5- |
| | 90:10 | counterion soln., stirred at 40° C., in solution after | MAPB |
| | | overnight stirring, opened for evaporation, solid | Pattern |
| | | after 6 days of evaporation, analyzed by PXRD | 9A |
| Maleic | MeOH:water | 40 mg 5-MAPB + 10 vols. solvent + 1:1 molar ratio | RS-5- |
| | 90:10 | counterion soln., stirred at 40° C., in solution after | MAPB |
| | | overnight stirring, opened for evaporation, solid | Pattern |
| | | after 7 days of evaporation, analyzed by PXRD | 10A |
| Malic | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | 6 days evaporation, terminated | |
| Citric | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | 6 days evaporation, terminated | |
| Fumaric | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | overnight evaporation, terminated | |
| Salicylic | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | overnight evaporation, terminated | |
| Hippuric | MeOH:water | 40 mg 5-MAPB + 10 vols. solvent + 1:1 molar ratio | N/A (gel) |
| | 90:10 | counterion soln., stirred at 40° C., in solution after | |
| | | overnight stirring, opened for evaporation, gel after | |
| | | overnight evaporation, terminated | |

In certain embodiments, the salt forms of RS-5-MAPB were prepared in a 1:1 molar ratio. In certain embodiments, the salt forms of RS-5-MAPB were prepared using excess amounts of RS-5-MAPB. In certain embodiments, the salt forms of RS-5-MAPB were prepared using excess amounts of salts. In certain embodiments, the solvent is acetone, methanol, water or methanol/water mixture.

In certain embodiments, salt forms of RS-5-MAPB were produced with counterions HCl, HBr, H₃PO₄, oxalic acid, and maleic acid. In certain embodiments, the solvents used to produce salt forms of RS-5-MAPB included acetone, MeOH:H₂O ratio. In certain embodiments, the ratio of methanol to water is 9:1.

In certain embodiments, Pattern 1A is produced from HCl and acetone. In certain embodiments, Pattern 1A is produced from HCl and MeOH:H₂O ratio. In certain embodiments, Pattern 1A is produced from HCl and MeOH:H₂O 90:10 ratio.

In certain embodiments, Pattern 4A is produced from H₃PO₄ and acetone. In certain embodiments, Pattern 4A is produced from H₃PO₄ and MeOH:H₂O ratio. In certain embodiments, Pattern 4A is produced from H₃PO₄ and MeOH:H₂O 90:10 ratio.

In certain embodiments, Pattern 9A is produced from oxalic acid and acetone. In certain embodiments, Pattern 9A is produced from oxalic acid and MeOH:H₂O ratio. In certain embodiments, Pattern 9A is produced from oxalic acid and MeOH:H₂O 90:10 ratio.

In certain embodiments, Pattern 10A is produced from maleic acid and acetone. In certain embodiments, Pattern 10A is produced from maleic acid and MeOH:H₂O ratio. In certain embodiments, Pattern 10A is produced from maleic acid and MeOH:H₂O 90:10 ratio.

In certain embodiments, Pattern 2A is produced from HBr and MeOH:H₂O ratio. In certain embodiments, Pattern 2A is produced from HBr and MeOH:H₂O 90:10 ratio.

### EXAMPLE 14. Salt Studies of 5-MAPB in DCM or EtOH:H₂O

Additional salt studies of 5-MAPB were conducted as shown below in Table 10. A total of 30 salt studies for 15 counterions in 2 different solvents, DCM and EtOH:H₂O (90:10), were generated from RS-5-MAPB HCl. The below technique was used to generate the XRPD patterns in FIG. 17 and FIG. 18. (For 40 mg of 5-MAPB, 1 vol. solvent=40 μL).

TABLE 10
Salt screening experiments

| | | | PXRD |
|---|---|---|---|
| Counterion | Solvent | Procedure/Comments | Result |
| HCl | DCM | 40 mg 5-MAPB + 10 vols. solvent + 1:1 molar | RS-5- |
| | | ratio counterion soln., stirred at RT, in solution | MAPB |
| | | after overnight stirring, opened for evaporation, | Pattern 1A |
| | | solid after 4 days of evaporation at RT, analyzed | |
| | | by PXRD | |
| HBr | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | gel after 4 days of evaporation at RT, terminated | |
| H₂SO₄ | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | gel after 4 days of evaporation at RT, terminated | |
| H₃PO₄ | DCM | 40 mg 5-MAPB + 10 vols. solvent + 1:1 molar | RS-5- |
| | | ratio counterion soln., stirred at RT, slurry after | MAPB |
| | | overnight stirring, centrifuged to harvest solids, | Pattern 4B |
| | | analyzed by PXRD, dried overnight at 40° C. in | |
| | | vacuum oven, analyzed by DSC and TGA | |
| HNO₃ | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | gel after 4 days of evaporation at RT, terminated | |
| Methane | DCM | 40 mg 5-MAPB + 10 vols. solvent + 1:1 molar | N/A (gel) |
| sulfonic | | ratio counterion soln., stirred at RT, in solution | |
| | | after overnight stirring, opened for evaporation, | |
| | | gel after 4 days of evaporation at RT, terminated | |
| Tartaric | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | temp increased to 40° C. after 6 days of | |
| | | evaporation at RT, gel after 10 days of | |
| | | evaporation, terminated | |
| Succinic | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | temp increased to 40° C. after 6 days of | |
| | | evaporation at RT, gel after 10 days of | |
| | | evaporation, terminated | |
| Oxalic | DCM | 40 mg 5-MAPB + 10 vols. solvent + 1:1 molar | RS-5- |
| | | ratio counterion soln., stirred at RT, centrifuged | MAPB |
| | | to harvest solids, analyzed by PXRD | Pattern 9A |
| Maleic | DCM | 40 mg 5-MAPB + 10 vols. solvent + 1:1 molar | RS-5- |
| | | ratio counterion soln., stirred at RT, in solution | MAPB |
| | | after overnight stirring, opened for evaporation, | Pattern |
| | | temp increased to 40° C. after 6 days of | 10A |
| | | evaporation at RT, solid after 10 days of | |
| | | evaporation, analyzed by PXRD | |
| Malic | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | temp increased to 40° C. after 6 days of | |
| | | evaporation at RT, gel after 10 days of | |
| | | evaporation, terminated | |
| Citric | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | temp increased to 40° C. after 6 days of | |
| | | evaporation at RT, gel after 10 days of | |
| | | evaporation, terminated | |
| Fumaric | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | gel after 4 days of evaporation at RT, terminated | |
| Salicylic | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | gel after 4 days of evaporation at RT, terminated | |
| Hippuric | DCM | 40 mg 5-MAPB + 10 vols. solvent + 1:1 molar | N/A (gel) |
| | | ratio counterion soln., stirred at RT, in solution | |
| | | after overnight stirring, opened for evaporation, | |
| | | gel after 4 days of evaporation at RT, terminated | |
| HCl | EtOH:water | 40 mg 5-MAPB + 10 vols. solvent + 1:1 molar | RS-5- |
| | 90:10 | ratio counterion soln., stirred at RT, in solution | MAPB |
| | | after overnight stirring, opened for evaporation, | Pattern 1A |
| | | solid after 4 days of evaporation at RT, analyzed | |
| | | by PXRD | |
| HBr | EtOH:water | 40 mg 5-MAPB + 10 vols. solvent + 1:1 molar | RS-5- |
| | 90:10 | ratio counterion soln., stirred at RT, in solution | MAPB |
| | | after overnight stirring, opened for evaporation, | Pattern 2A |
| | | solid after 4 days of evaporation at RT, analyzed | |
| | | by PXRD | |
| H₂SO₄ | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | gel after 4 days of evaporation at RT, terminated | |
| H₃PO₄ | EtOH:water | 40 mg 5-MAPB + 10 vols. solvent + 1:1 molar | RS-5- |
| | 90:10 | ratio counterion soln., stirred at RT, in solution | MAPB |
| | | after overnight stirring, opened for evaporation, | Pattern 4B |
| | | solid after 4 days of evaporation at RT, | |
| | | deliquesced during PXRD sample prep, reopened | |
| | | for evaporation, temp increased to 40° C. after 6 | |
| | | days of evaporation at RT, solid after 7 days of | |
| | | evaporation, analyzed by PXRD, dried overnight | |
| | | at 40° C. in vacuum oven | |
| HNO₃ | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | gel after 4 days of evaporation at RT, terminated | |
| Methane | EtOH:water | 40 mg 5-MAPB + 10 vols. solvent + 1:1 molar | N/A (gel) |
| sulfonic | 90:10 | ratio counterion soln., stirred at RT, in solution | |
| | | after overnight stirring, opened for evaporation, | |
| | | gel after 4 days of evaporation at RT, terminated | |
| Tartaric | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | temp increased to 40° C. after 6 days of | |
| | | evaporation at RT, gel after 10 days of | |
| | | evaporation, terminated | |
| Succinic | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | temp increased to 40° C. after 6 days of | |
| | | evaporation at RT, gel after 10 days of | |
| | | evaporation, terminated | |
| Oxalic | EtOH:water | 40 mg 5-MAPB + 10 vols. solvent + 1:1 molar | RS-5- |
| | 90:10 | ratio counterion soln., stirred at RT, in solution | MAPB |
| | | after overnight stirring, opened for evaporation, | Pattern 9A |
| | | temp increased to 40° C. after 6 days of | |
| | | evaporation at RT, solid after 7 days of | |
| | | evaporation, analyzed by PXRD | |
| Maleic | EtOH:water | 40 mg 5-MAPB + 10 vols. solvent + 1:1 molar | RS-5- |
| | 90:10 | ratio counterion soln., stirred at RT, in solution | MAPB |
| | | after overnight stirring, opened for evaporation, | Pattern |
| | | temp increased to 40° C. after 6 days of | 10A |
| | | evaporation at RT, solid after 10 days of | |
| | | evaporation, analyzed by PXRD | |
| Malic | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | temp increased to 40° C. after 6 days of | |
| | | evaporation at RT, gel after 10 days of | |
| | | evaporation, terminated | |
| Citric | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | temp increased to 40° C. after 6 days of | |
| | | evaporation at RT, gel after 10 days of | |
| | | evaporation, terminated | |
| Fumaric | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | gel after 4 days of evaporation at RT, terminated | |
| Salicylic | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | gel after 4 days of evaporation at RT, terminated | |
| Hippuric | EtOH:water | 40 mg 5-MAPB + 10 vols. solvent + 1:1 molar | N/A (gel) |
| | 90:10 | ratio counterion soln., stirred at RT, in solution | |
| | | after overnight stirring, opened for evaporation, | |
| | | gel after 4 days of evaporation at RT, terminated | |

In certain embodiments, the salt forms of RS-5-MAPB were prepared in a 1:1 molar ratio. In certain embodiments, the salt forms of RS-5-MAPB were prepared using excess amounts of RS-5-MAPB. In certain embodiments, the salt forms of RS-5-MAPB were prepared using excess amounts of salts. In certain embodiments, the solvent is DCM, ethanol, water or ethanol/water mixture.

In certain embodiments, salt forms of RS-5-MAPB were produced with counterions HCl, HBr, H₃PO₄, oxalic acid, and maleic acid. In certain embodiments, the solvents used to produce salt forms of RS-5-MAPB included dichloromethane (DCM) and EtOH:H₂O ratio. In certain embodiments, the ratio of ethanol to water is 9:1.

In certain embodiments, Pattern 1A is produced from HCl and DCM. In certain embodiments, Pattern 1A is produced from HCl and EtOH:H₂O ratio. In certain embodiments, Pattern 1A is produced from HCl and EtOH:H₂O 90:10 ratio.

In certain embodiments, Pattern 4B is produced from H₃PO₄ and DCM. In certain embodiments, Pattern 4B is produced from H₃PO₄ and EtOH:H₂O ratio. In certain embodiments, Pattern 4B is produced from H₃PO₄ and EtOH:H₂O 90:10 ratio.

In certain embodiments, Pattern 9A is produced from oxalic acid and DCM. In certain embodiments, Pattern 9A is produced from oxalic acid and EtOH:H₂O ratio. In certain embodiments, Pattern 9A is produced from oxalic acid and EtOH:H₂O 90:10 ratio.

In certain embodiments, Pattern 10A is produced from maleic acid and DCM. In certain embodiments, Pattern 10A is produced from maleic acid and EtOH:H₂O ratio. In certain embodiments, Pattern 10A is produced from maleic acid and EtOH:H₂O 90:10 ratio.

In certain embodiments, Pattern 2A is produced from HBr and EtOH:H₂O ratio. In certain embodiments, Pattern 2A is produced from HBr and EtOH:H₂O 90:10 ratio.

### EXAMPLE 15. Salt Studies of 5-MAPB in THF

Salt studies of 5-MAPB were conducted as shown below in Table 11. A total of 30 salt screening experiments of 15 counterions in THE were generated from RS-5-MAPB HCl. The below technique was used to generate the XRPD patterns in FIG. 19. (For 40 mg of 5-MAPB, 1 vol. solvent=40 μL).

TABLE 11
Salt screening experiments

| | | | PXRD |
|---|---|---|---|
| Counterion | Solvent | Procedure/Comments | Result |
| HBr | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| H₂SO₄ | THF | 50 mg 5-MAPB + 10 vols. solvent + 1:0.5 molar | N/A (gel) |
| | | ratio counterion soln. (0.5 eq. counterion), stirred | |
| | | at 40° C., was in solution, opened for evaporation | |
| | | overnight, was amorphous gel, terminated | |
| H₂SO₄ | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| H₃PO₄ | THF | 50 mg 5-MAPB + 10 vols. solvent + 1:0.33 molar | N/A (gel) |
| | | ratio counterion soln. (0.33 eq. counterion), stirred | |
| | | at 40° C., was slurry after overnight stirring at 40 | |
| | | ° C., centrifuged, solids deliquesced, terminated | |
| H₃PO₄ | THF | 50 mg 5-MAPB + 10 vols. solvent + 1:0.5 molar | RS-5- |
| | | ratio counterion soln. (0.5 eq. counterion), stirred | MAPB |
| | | at 40° C., was slurry, centrifuged and analyzed by | Pattern |
| | | PXRD, dried overnight at 40° C. in vacuum oven, | 4C |
| | | analyzed by DSC and TGA | |
| H₃PO₄ | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| HNO₃ | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| Methane | THF | 40 mg 5-MAPB + 10 vols. solvent + 1:1 molar | N/A (gel) |
| sulfonic | | ratio counterion soln., stirred at 40° C., was in | |
| | | solution, opened for evaporation overnight, was | |
| | | amorphous gel, terminated | |
| Tartaric | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| Succinic | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| Malic | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| Citric | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| Fumaric | THF | 40 mg 5-MAPB + 10 vols. solvent + 1:1 molar | N/A (gel) |
| | | ratio counterion soln., stirred at 40° C., was slurry | |
| | | after overnight stirring at 40° C., centrifuged, solids | |
| | | deliquesced, terminated | |
| Salicylic | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |
| Hippuric | THF | 40 mg 5-MAPB + 10 vols. solvent + 1:1 molar | N/A (gel) |
| | | ratio counterion soln., stirred at 40° C., was in | |
| | | solution, opened for evaporation overnight, was | |
| | | amorphous gel, terminated | |

In certain embodiments, the salt forms of RS-5-MAPB were prepared in a 1:1 molar ratio. In certain embodiments, the salt forms of RS-5-MAPB were prepared using excess amounts of RS-5-MAPB. In certain embodiments, the salt forms of RS-5-MAPB were prepared using excess amounts of salts.

In certain embodiments, salt forms of RS-5-MAPB were produced with counterions HCl, HBr, H₃PO₄, oxalic acid, and maleic acid. In certain embodiments, the solvents used to produce salt forms of RS-5-MAPB included tetrahydrofuran (THF).

In certain embodiments, Pattern 4C is produced from H₃PO₄ and THF.

### EXAMPLE 16. 5-MAPB Freebase Isolation/Liquid-Liquid Extraction

Liquid-Liquid Extraction (LLE) was used to isolate 5-MAPB Freebase from Pattern 1A (5-MAPB HCl, Pure Enantiomer) using the conditions shown in Table 12 (for 2 g of 5-MAPB HCl Pure Enantiomer, 1 vol. solvent is equivalent to 2 mL).

TABLE 12
S-5-MAPB Freebase from Pattern 1A Enantiomer (S-5-MAPB Pure Enantiomer)

| | Solvent | |
|---|---|---|
| Counterion | (Density) | Procedure/Comments |
| NaOH | EtOAc | 2 g Pattern 1A Enantiomer (as HCl salt) + 10 vols. solvent + NaOH |
| | (0.902 | stock soln. in water (1.1:1 molar ratio) + additional water (10 vols. |
| | g/mL) | water total), did not dissolve in 10 vols. of EtOAc, went into solution |
| | | and changed color after adding NaOH, EtOAc phase removed |

### EXAMPLE 17. Salt Studies of Pattern 1A S-5-MAPB in Acetone or MeOH:H₂O

Salt studies of S-5-MAPB HCl Pattern 1A Enantiomer were conducted as shown below in Table 13. A total of 24 salt experiments for 12 counterions (HCl, HBr, H₂SO₄, H₃PO₄, HNO₃, methansulfonic, succinic, oxalic, maleic, fumaric, L-arginine, L-lysine) in 2 different solvents, acetone and MeOH:H₂O, were generated S-5-MAPB HCl Pattern 1A. The below technique was used to generate the images in FIG. 24, FIG. 25 and FIG. 26. (for 35 mg of S-5-MAPB HCl Pattern 1A, 1 vol. solvent=35 μL, (API=S-5-MAPB HCl Pattern 1A)).

TABLE 13
Salt screening experiments of Pattern 1A Enantiomer (S-5-MAPB Pure
Enantiomer)

| | | | PXRD |
|---|---|---|---|
| Counterion | Solvent | Procedure/Comments | Result |
| HCl | Acetone | 35 mg API + 10 vols. solvent + 1:1 molar ratio | S-5-MAPB |
| | | counterion soln., stirred at 40° C., slurry after | Pattern 1A |
| | | overnight stirring, centrifuged to recover solids, | |
| | | analyzed by PXRD, dried overnight at 40° C. in | |
| | | vacuum oven, analyzed by DSC and TGA | |
| HBr | Acetone | 35 mg API + 10 vols. solvent + 1:1 molar ratio | S-5-MAPB |
| | | counterion soln., stirred at 40° C., in solution after | Pattern 2A |
| | | overnight stirring, opened for evaporation, solid after | |
| | | 3 days of evaporation, analyzed by PXRD, dried | |
| | | overnight at 40° C. in vacuum oven, analyzed by DSC | |
| | | and TGA | |
| H₂SO₄ | Acetone | 35 mg API + 10 vols. solvent + 1:1 molar ratio | N/A (gel) |
| | | counterion soln., stirred at 40° C., in solution after | |
| | | overnight stirring, opened for evaporation, gel after 3 | |
| | | days of evaporation, terminated | |
| H₃PO₄ | Acetone | 35 mg API + 10 vols. solvent + 1:1 molar ratio | S-5-MAPB |
| | | counterion soln., stirred at 40° C., slurry after | Pattern 4A |
| | | overnight stirring, centrifuged to recover solids, | |
| | | analyzed by PXRD, dried overnight at 40° C. in | |
| | | vacuum oven, analyzed by DSC and TGA | |
| HNO₃ | Acetone | 35 mg API + 10 vols. solvent + 1:1 molar ratio | N/A (gel) |
| | | counterion soln., stirred at 40° C., in solution after | |
| | | overnight stirring, opened for evaporation, gel after 3 | |
| | | days of evaporation, terminated | |
| Methane | Acetone | 35 mg API + 10 vols. solvent + 1:1 molar ratio | N/A (gel) |
| Sulfonic | | counterion soln., stirred at 40° C., in solution after | |
| | | overnight stirring, opened for evaporation, gel after 3 | |
| | | days of evaporation, terminated | |
| Succinic | Acetone | 35 mg API + 10 vols. solvent + 1:1 molar ratio | N/A (gel) |
| | | counterion soln., stirred at 40° C., in solution after | |
| | | overnight stirring, opened for evaporation, gel after 4 | |
| | | days of evaporation, terminated | |
| Oxalic | Acetone | 35 mg API + 10 vols. solvent + 1:1 molar ratio | S-5-MAPB |
| | | counterion soln., stirred at 40° C., in solution after | Pattern 8A |
| | | overnight stirring, opened for evaporation, solid after | |
| | | 3 days of evaporation, analyzed by PXRD, dried | |
| | | overnight at 40° C. in vacuum oven, analyzed by DSC | |
| | | and TGA | |
| Maleic | Acetone | 35 mg API + 10 vols. solvent + 1:1 molar ratio | N/A (gel) |
| | | counterion soln., stirred at 40° C., in solution after | |
| | | overnight stirring, opened for evaporation, gel after 4 | |
| | | days of evaporation, terminated | |
| Fumaric | Acetone | 35 mg API + 10 vols. solvent + 1:1 molar ratio | N/A (gel) |
| | | counterion soln., stirred at 40° C., in solution after | |
| | | overnight stirring, opened for evaporation, gel after 3 | |
| | | days of evaporation, terminated | |
| L-Arginine | Acetone | 35 mg API + 10 vols. solvent + 1:1 molar ratio | N/A (gel) |
| | | counterion soln., stirred at 40° C., in solution after | |
| | | overnight stirring, opened for evaporation, gel after 6 | |
| | | days of evaporation, terminated | |
| L-Lysine | Acetone | 35 mg API + 10 vols. solvent + 1:1 molar ratio | N/A (gel) |
| | | counterion soln., stirred at 40° C., in solution after | |
| | | overnight stirring, opened for evaporation, gel after 5 | |
| | | days of evaporation, terminated | |
| HCl | MeOH:water | 35 mg API + 10 vols. solvent + 1:1 molar ratio | S-5-MAPB |
| | 90:10 | counterion soln., stirred at 40° C., in solution after | Pattern 1A |
| | | overnight stirring, opened for evaporation, solid after | |
| | | 3 days of evaporation, analyzed by PXRD, dried | |
| | | overnight at 40° C. in vacuum oven, analyzed by DSC | |
| | | and TGA | |
| HBr | MeOH:water | 35 mg API + 10 vols. solvent + 1:1 molar ratio | S-5-MAPB |
| | 90:10 | counterion soln., stirred at 40° C., in solution after | Pattern 2A |
| | | overnight stirring, opened for evaporation, solid after | |
| | | 3 days of evaporation, analyzed by PXRD, dried | |
| | | overnight at 40° C. in vacuum oven | |
| H₂SO₄ | MeOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after 3 | |
| | | days of evaporation, terminated | |
| H₃PO₄ | MeOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after 3 | |
| | | days of evaporation, terminated | |
| HNO₃ | MeOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after 3 | |
| | | days of evaporation, terminated | |
| Methane | MeOH:water | 35 mg API + 10 vols. solvent + 1:1 molar ratio | N/A (gel) |
| Sulfonic | 90:10 | counterion soln., stirred at 40° C., in solution after | |
| | | overnight stirring, opened for evaporation, gel after 3 | |
| | | days of evaporation, terminated | |
| Succinic | MeOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after 5 | |
| | | days of evaporation, terminated | |
| Oxalic | MeOH:water | 35 mg API + 10 vols. solvent + 1:1 molar ratio | S-5-MAPB |
| | 90:10 | counterion soln., stirred at 40° C., in solution after | Pattern 8A |
| | | overnight stirring, opened for evaporation, solid after | |
| | | 3 days of evaporation, analyzed by PXRD | |
| Maleic | MeOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after 5 | |
| | | days of evaporation, terminated | |
| Fumaric | MeOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after 3 | |
| | | days of evaporation, terminated | |
| L-Arginine | MeOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after 6 | |
| | | days of evaporation, terminated | |
| L-Lysine | MeOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after 6 | |
| | | days of evaporation, terminated | |

In certain embodiments, the salt forms of S-5-MAPB were prepared in a 1:1 molar ratio. In certain embodiments, the salt forms of S-5-MAPB were prepared using excess amounts of S5-5-MAPB. In certain embodiments, the salt forms of S-5-MAPB were prepared using excess amounts of salts.

In certain embodiments, salt forms of S-5-MAPB were produced with counterions HCl, HBr, H₂SO₄, H₃PO₄, HNO₃, Methansulfonic, Succinic, Oxalic, Maleic, Fumaric, L-Arginine, and L-Lysine. In certain embodiments, the solvent is acetone, methanol, water or methanol/water mixture.

In certain embodiments, Pattern 1A Enantiomer (Pattern 1AE) is produced from HCl and acetone. In certain embodiments, Pattern 1A Enantiomer (Pattern 1AE) is produced from HCl and MeOH:H₂O ratio. In certain embodiments, Pattern 1A Enantiomer (Pattern 1AE) is produced from HCl and MeOH:H₂O 90:10 ratio.

In certain embodiments, Pattern 4A Enantiomer (Pattern 4AE) is produced from H₃PO₄ and acetone.

In certain embodiments, Pattern 8A Enantiomer (Pattern 8AE) is produced from oxalic acid and acetone. In certain embodiments, Pattern 8A Enantiomer (Pattern 8AE) is produced from oxalic acid and MeOH:H₂O ratio. In certain embodiments, Pattern 8AE Enantiomer (Pattern 8AE) is produced from oxalic acid and MeOH:H₂O 90:10 ratio.

In certain embodiments, Pattern 2A Enantiomer (Pattern 2AE) is produced from HBr and acetone. In certain embodiments, Pattern 2A Enantiomer (Pattern 2AE) is produced from HBr and MeOH:H₂O ratio. In certain embodiments, Pattern 2AE is produced from HBr and MeOH:H₂O 90:10 ratio.

### EXAMPLE 18. Salt Screening Experiments of Pattern 1A S-5-MAPB Pure Enantiomer in THF or EtOH:H₂O

Salt screening experiments of Pattern 1A Enantiomer (S-5-MAPB Pure Enantiomer) were conducted as shown below in Table 14. A total of 24 salt screening experiments of 12 counterions (HCl, HBr, H₂SO₄, H₃PO₄, HNO₃, methansulfonic, succinic, oxalic, maleic, fumaric, L-arginine, L-lysine) in 2 different solvents, THE and EtOH:H₂O, were generated from Pattern 1A Enantiomer (S-5-MAPB Pure Enantiomer). The below technique was used to generate the images in FIG. 27, FIG. 28, FIG. 29 and FIG. 30. For 35 mg of Pattern 1A Enantiomer, 1 vol. solvent=35 μL; (API=Pattern 1A Enantiomer (S-5-MAPB Pure Enantiomer)).

TABLE 14
Salt screening experiments of Pattern 1A Enantiomer (S-5-MAPB Pure
Enantiomer)

| Counterion | Solvent | Procedure/Comments | PXRD Result |
|---|---|---|---|
| HCl | THF | 35 mg API + 10 vols. solvent + 1:1 molar ratio | S-5-MAPB |
| | | counterion soln., stirred at 40° C., slurry after | Pattern 1A |
| | | overnight stirring, centrifuged to recover solids, | |
| | | analyzed by PXRD | |
| HBr | THF | 35 mg API + 10 vols. solvent + 1:1 molar ratio | S-5-MAPB |
| | | counterion soln., stirred at 40° C., in solution after | Pattern 2A |
| | | overnight stirring, opened for evaporation, solid | |
| | | after overnight evaporation, analyzed by PXRD | |
| H₂SO₄ | THF | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 5 days of evaporation, terminated | |
| H₃PO₄ | THF | 35 mg API + 10 vols. solvent + 1:1 molar ratio | S-5-MAPB |
| | | counterion soln., stirred at 40° C., slurry after | Pattern 4A |
| | | overnight stirring, centrifuged to recover solids, | |
| | | analyzed by PXRD | |
| HNO₃ | THF | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | overnight evaporation, terminated | |
| Methane | THF | 35 mg API + 10 vols. solvent + 1:1 molar ratio | N/A (gel) |
| Sulfonic | | counterion soln., stirred at 40° C., in solution after | |
| | | overnight stirring, opened for evaporation, gel after | |
| | | overnight evaporation, terminated | |
| Succinic | THF | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 2 days of evaporation, terminated | |
| Oxalic | THF | 35 mg API + 10 vols. solvent + 1:1 molar ratio | S-5-MAPB |
| | | counterion soln., stirred at 40° C., in solution after | Pattern 8A |
| | | overnight stirring, opened for evaporation, solid | |
| | | after 2 days of evaporation, analyzed by PXRD | |
| Maleic | THF | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 2 days of evaporation, terminated | |
| Fumaric | THF | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 2 days of evaporation, terminated | |
| L-Arginine | THF | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 5 days of evaporation, terminated | |
| L-Lysine | THF | 35 mg API + 10 vols. solvent + 1:1 molar ratio | L-Lysine |
| | | counterion soln., stirred at 40° C., in solution after | |
| | | overnight stirring, opened for evaporation, solid | |
| | | after 5 days of evaporation, analyzed by PXRD | |
| HCl | EtOH:water | 35 mg API + 10 vols. solvent + 1:1 molar ratio | S-5-MAPB |
| | 90:10 | counterion soln., stirred at 40° C., in solution after | Pattern 1A |
| | | overnight stirring, opened for evaporation, solid | |
| | | after overnight evaporation, analyzed by PXRD | |
| HBr | EtOH:water | 35 mg API + 10 vols. solvent + 1:1 molar ratio | S-5-MAPB |
| | 90:10 | counterion soln., stirred at 40° C., in solution after | Pattern 2A |
| | | overnight stirring, opened for evaporation, solid | |
| | | after 2 days of evaporation, analyzed by PXRD, | |
| | | dried overnight at 40° C. in vacuum oven, analyzed | |
| | | by DSC and TGA | |
| H₂SO₄ | EtOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after | |
| | | 2 days of evaporation, terminated | |
| H₃PO₄ | EtOH:water | 35 mg API + 10 vols. solvent + 1:1 molar ratio | S-5-MAPB |
| | 90:10 | counterion soln., stirred at 40° C., in solution after | Pattern 4A |
| | | overnight stirring, opened for evaporation, solid | |
| | | after overnight evaporation, analyzed by PXRD | |
| HNO₃ | EtOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after | |
| | | 2 days of evaporation, terminated | |
| Methane | EtOH:water | 35 mg API + 10 vols. solvent + 1:1 molar ratio | N/A (gel) |
| Sulfonic | 90:10 | counterion soln., stirred at 40° C., in solution after | |
| | | overnight stirring, opened for evaporation, gel after | |
| | | 2 days of evaporation, terminated | |
| Succinic | EtOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after | |
| | | 5 days of evaporation, terminated | |
| Oxalic | EtOH:water | 35 mg API + 10 vols. solvent + 1:1 molar ratio | S-5-MAPB |
| | 90:10 | counterion soln., stirred at 40° C., in solution after | Pattern 8A |
| | | overnight stirring, opened for evaporation, solid | |
| | | after 2 days of evaporation, analyzed by PXRD | |
| Maleic | EtOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after | |
| | | 5 days of evaporation, terminated | |
| Fumaric | EtOH:water | 35 mg API + 10 vols. solvent + 1:1 molar ratio | S-5-MAPB |
| | 90:10 | counterion soln., stirred at 40° C., in solution after | Pattern 10A |
| | | overnight stirring, opened for evaporation, solid | |
| | | after 5 days of evaporation, analyzed by PXRD, | |
| | | dried overnight at 40° C. in vacuum oven, analyzed | |
| | | by DSC and TGA | |
| L-Arginine | EtOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after | |
| | | 5 days of evaporation, terminated | |
| L-Lysine | EtOH:water | 35 mg API + 10 vols. solvent + 1:1 molar ratio | N/A (gel) |
| | 90:10 | counterion soln., stirred at 40° C., in solution after | |
| | | overnight stirring, opened for evaporation, gel after | |
| | | 5 days of evaporation, terminated | |

In certain embodiments, the salt forms of S-5-MAPB were prepared in a 1:1 molar ratio. In certain embodiments, the salt forms of S-5-MAPB were prepared using excess amounts of S-5-MAPB. In certain embodiments, the salt forms of S-5-MAPB were prepared using excess amounts of salts.

In certain embodiments, salt forms of S-5-MAPB were produced with counterions HCl, HBr, H₂SO₄, H₃PO₄, HNO₃, Methansulfonic, Succinic, Oxalic, Maleic, Fumaric, L-Arginine, and L-Lysine. In certain embodiments, the solvent is tetrahydrofuran, ethanol, water or ethanol/water mixture.

In certain embodiments, Pattern 1A Enantiomer (Pattern 1AE) is produced from HCl and THF. In certain embodiments, Pattern 1A Enantiomer (Pattern 1AE) is produced from HCl and EtOH:H₂O ratio. In certain embodiments, Pattern 1A Enantiomer (Pattern 1AE) is produced from HCl and EtOH:H₂O 90:10 ratio.

In certain embodiments, Pattern 4A Enantiomer (Pattern 4AE) is produced from H₃PO₄ and THF. In certain embodiments, Pattern 4A Enantiomer (Pattern 4AE) is produced from HCl and EtOH:H₂O ratio. In certain embodiments, Pattern 4A Enantiomer (Pattern 4AE) is produced from HCl and EtOH:H₂O 90:10 ratio.

In certain embodiments, Pattern 8A Enantiomer (Pattern 8AE) is produced from oxalic acid and THF. In certain embodiments, Pattern 8A Enantiomer (Pattern 8AE) is produced from oxalic acid and EtOH:H₂O ratio. In certain embodiments, Pattern 8A Enantiomer (Pattern 8AE) is produced from oxalic acid and EtOH:H₂O 90:10 ratio.

In certain embodiments, Pattern 2A Enantiomer (Pattern 2AE) is produced from HBr and THF. In certain embodiments, Pattern 2A Enantiomer (Pattern 2AE) is produced from HBr and EtOH:H₂O ratio. In certain embodiments, Pattern 2AE is produced from HBr and EtOH:H₂O 90:10 ratio.

In certain embodiments, Pattern 10A Enantiomer (Pattern 10AE) is produced from fumaric acid and EtOH:H₂O ratio. In certain embodiments, Pattern 10AE is produced from fumaric acid and EtOH:H₂O 90:10 ratio.

### EXAMPLE 19. Salt Screening Experiments of Pattern 1A S-5-MAPB Pure Enantiomer in ACN

Salt screening experiments of Pattern 1A Enantiomer (S-5-MAPB Pure Enantiomer) were conducted as shown below in Table 15. A total of 13 salt screening experiments of 10 counterions in 1 solvent (ACN, acetonitrile) were generated from Pattern 1A Enantiomer (S-5-MAPB Pure Enantiomer). The below technique was used to generate the images in FIG. 31. For 35 mg of Pattern 1A Enantiomer, 1 vol. solvent=35 μL; (API=Pattern 1A Enantiomer (S-5-MAPB Pure Enantiomer)).

TABLE 15
Salt screening experiments of Pattern 1A Enantiomer (S-5-MAPB Pure
Enantiomer)

| Counterion | Solvent | Procedure/Comments | PXRD Result |
|---|---|---|---|
| HCl | ACN | 35 mg API + 10 vols. solvent + 1:1 molar ratio | S-5-MAPB |
| | | counterion soln., stirred at 40° C., slurry after | Pattern 1A |
| | | overnight stirring, centrifuged to harvest solids, | |
| | | analyzed by PXRD | |
| HBr | ACN | 35 mg API + 10 vols. solvent + 1:1 molar ratio | S-5-MAPB |
| | | counterion soln., stirred at 40° C., in solution after | Pattern 2A |
| | | overnight stirring, opened for evaporation, solid | |
| | | after overnight evaporation, analyzed by PXRD | |
| H₂SO₄ | ACN | 35 mg API + 10 vols. solvent + 1:0.5 molar ratio | N/A (gel) |
| | | counterion soln. (0.5 eq. counterion), stirred at 40° | |
| | | C., in solution after overnight stirring, opened for | |
| | | evaporation, solid after overnight evaporation, | |
| | | became gel-like during PXRD sample prep, | |
| | | terminated | |
| H₂SO₄ | ACN | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 2 days of evaporation, terminated | |
| H₃PO₄ | ACN | 35 mg API + 10 vols. solvent + 1:0.33 molar ratio | N/A (gel) |
| | | counterion soln. (0.33 eq. counterion), stirred at 40° | |
| | | C., slurry after overnight stirring, centrifuged to | |
| | | harvest solids, became gel-like during PXRD | |
| | | sample prep, terminated | |
| H₃PO₄ | ACN | 35 mg API + 10 vols. solvent + 1:0.5 molar ratio | N/A (gel) |
| | | counterion soln. (0.5 eq. counterion), stirred at 40° | |
| | | C., in solution after overnight stirring, opened for | |
| | | evaporation, gel after 2 days of evaporation, | |
| | | terminated | |
| H₃PO₄ | ACN | 35 mg API + 10 vols. solvent + 1:1 molar ratio | S-5-MAPB |
| | | counterion soln., stirred at 40° C. slurry after | Pattern 4A |
| | | overnight stirring, centrifuged to harvest solids, | |
| | | analyzed by PXRD | |
| HNO₃ | ACN | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | overnight evaporation, terminated | |
| Methane | ACN | 35 mg API + 10 vols. solvent + 1:1 molar ratio | N/A (gel) |
| Sulfonic | | counterion soln., stirred at 40° C., in solution after | |
| | | overnight stirring, opened for evaporation, gel after | |
| | | overnight evaporation, terminated | |
| Succinic | ACN | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 5 days of evaporation, terminated | |
| Fumaric | ACN | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 2 days of evaporation, terminated | |
| L-Arginine | ACN | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 5 days of evaporation, terminated | |
| L-Lysine | ACN | 35 mg API + 10 vols. solvent + 1:1 molar ratio | N/A (gel) |
| | | counterion soln., stirred at 40° C., in solution after | |
| | | overnight stirring, opened for evaporation, gel after | |
| | | 5 days of evaporation, terminated | |

In certain embodiments, the salt forms of S-5-MAPB were prepared in a 1:1 molar ratio. In certain embodiments, the salt forms of S-5-MAPB were prepared using excess amounts of S-5-MAPB. In certain embodiments, the salt forms of S-5-MAPB were prepared using excess amounts of salts.

In certain embodiments, salt forms of S-5-MAPB were produced with counterions HCl, HBr, H₂SO₄, H₃PO₄, HNO₃, Methansulfonic, Succinic, Oxalic, Maleic, Fumaric, L-Arginine, and L-Lysine. In certain embodiments, the solvent is acetonitrile.

In certain embodiments, Pattern 1A Enantiomer (Pattern 1AE) is produced from HCl and acetonitrile (ACN).

In certain embodiments, Pattern 2A Enantiomer (Pattern 2AE) is produced from HBr and acetonitrile (ACN).

In certain embodiments, Pattern 4A Enantiomer (Pattern 4AE) is produced from H₃PO₄ and acetonitrile (ACN).

### EXAMPLE 20. Differential Scanning Calorimetry (DSC) Thermogram Procedure

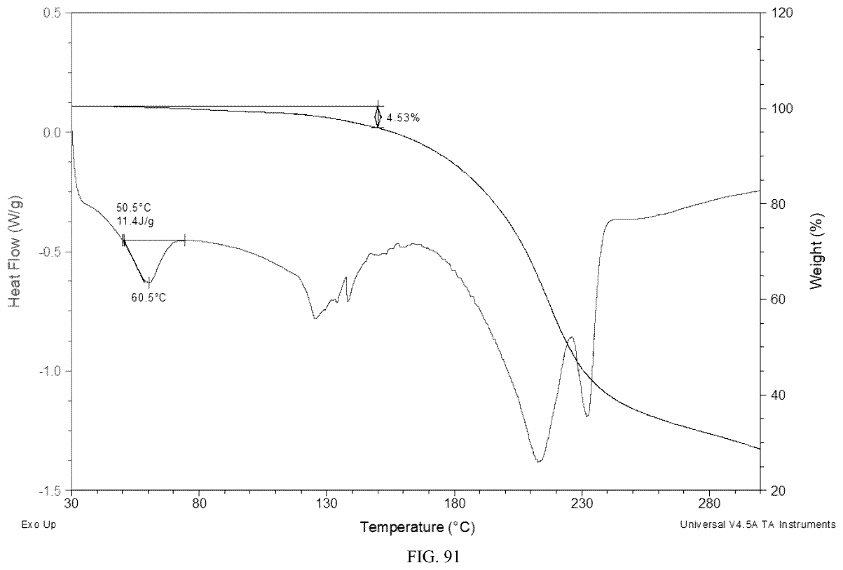

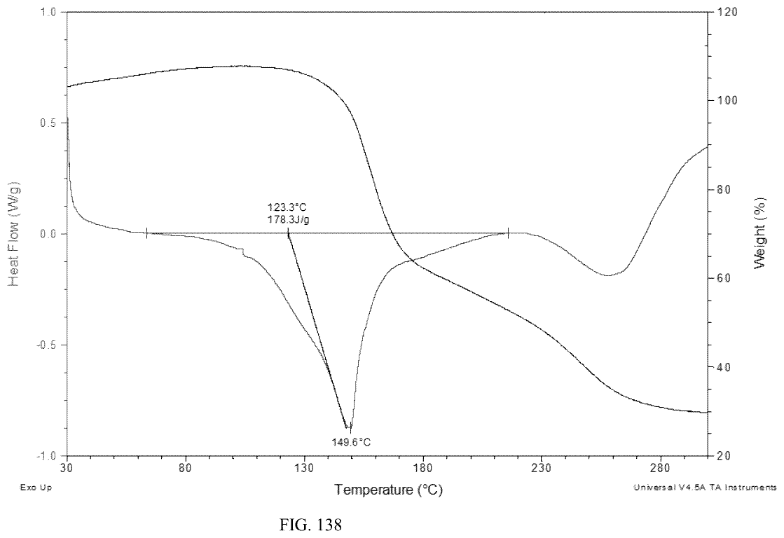

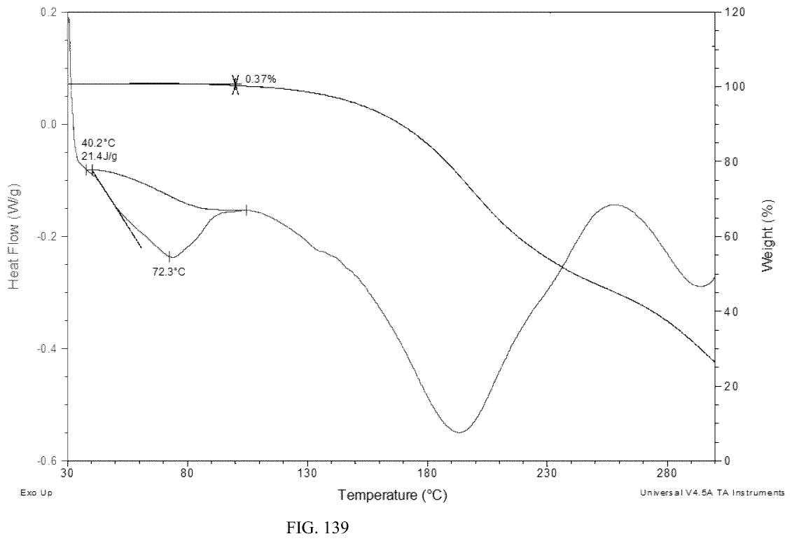

Differential Scanning Calorimetry (DSC) thermograms were collected on a Perkin Elmer Pyris 1 DSC with Intracooler. Thermogravimetric (TGA) thermograms were collected on a Perkin Elmer TGA-7 Instrument. The instrument and method details are included in the following table. The crystalline hits obtained during the salt screening experiments were further characterized by DSC and TGA. The instrument and method details are included below in the Table 16. The below technique was used to generate the images in FIG. 35, FIG. 36, FIG. 37, FIG. 38, FIG. 39, FIG. 40, FIG. 41, FIG. 42, FIG. 43, FIG. 44, FIG. 45, FIG. 46, FIG. 57, FIG. 58, FIG. 59, FIG. 60, and FIG. 61, FIG. 89, FIG. 90, FIG. 91, FIG. 92, FIG. 93, FIG. 94, FIG. 95, FIG. 96, FIG. 97, FIG. 99, FIG. 128, FIG. 129, FIG. 130, FIG. 131, FIG. 132, FIG. 133, FIG. 134, FIG. 135, FIG. 136, FIG. 137, FIG. 138, FIG. 139, FIG. 155, FIG. 156, FIG. 157, FIG. 158, FIG. 159, FIG. 160, and FIG. 161.

TABLE 16
DSC/TGA thermogram procedure

| | Perkin Elmer Pyris 1 | |
|---|---|---|
| | DSC with Intracooler | |
| Instrument: | (S/N 537N7063001) | Perkin Elmer TGA-7 |
| Sample Holder: | Thermal Support Standard | (S/N 519N7100203) |
| | 6.7 mm Al Pans | |
| Scan Temp | 30° C. to 250° C. | Mettler Toledo Al |
| Range: | | Crucibles 40 μL |
| Scan Rate: | 10° C./min | 30° C. to 300° C. |
| Purge: | Nitrogen, 20 cc/min | 10° C./min |

### EXAMPLE 21. Scale-Up and Stability Study

Scale up study of selected S-5-MAPB salts (Patterns 1A, 2A, and 10A) to −70 mg was completed. Patterns 1A and 10A were successfully scaled up, however the attempt to scale up Pattern 2A was unsuccessful (new Pattern 2B was obtained instead). All three samples were then tested for their solid-state stability as shown below in Table 17. (API=5-MAPB HCl)

TABLE 17
Scale Up and Stability of Pattern 1A, 2A and 10A

| | | | | | PXRD |
|---|---|---|---|---|---|
| | | | | PXRD | Pattern |
| | | | PXRD | Pattern | (40° |
| | | | Pattern | (Vac. | C./75% |
| Counter | | | (Wet | dried | RH |
| ion | Solvent | Procedure/Comments | cake) | sample) | sample) |
| HCl | EtOH: | 70 mg API + 10 vols. solvent + 1:1 | S-5- | S-5- | S-5- |
| Pattern | water | molar ratio counterion soln., stirred | MAPB | MAPB | MAPB |
| 1A) | 90:10 | at 40° C., in solution after ~6 hrs of | Pattern | Pattern | Pattern 1A |
| | | stirring, opened for evaporation, | 1A | 1A | |
| | | solid after 2 days of evaporation, | | | |
| | | analyzed by PXRD, dried overnight | | | |
| | | at 40° C. in vacuum oven, analyzed | | | |
| | | by PXRD, DSC, and TGA, | | | |
| | | analyzed by optical microscopy, | | | |
| | | ~10 mgs staged at 40° C./75% RH, | | | |
| | | pulled from staging after 1 day, | | | |
| | | analyzed by PXRD | | | |
| HBr | EtOH: | 70 mg API + 10 vols. solvent + 1:1 | S-5- | S-5- | S-5- |
| (Pattern | water | molar ratio counterion soln., stirred | MAPB | MAPB | MAPB |
| 2A) | 90:10 | at 40° C., in solution after ~6 hrs of | Pattern | Pattern | Pattern 2B |
| | | stirring, opened for evaporation, | 2B | 2B | |
| | | solid after 2 days of evaporation, | | | |
| | | analyzed by PXRD, dried overnight | | | |
| | | at 40° C. in vacuum oven, analyzed | | | |
| | | by PXRD, DSC, and TGA, | | | |
| | | analyzed by optical microscopy, | | | |
| | | ~10 mgs staged at 40° C./75% RH, | | | |
| | | pulled from staging after 1 day, | | | |
| | | analyzed by PXRD | | | |
| Maleic | DCM | 65 mg API + 10 vols. solvent + 1:1 | S-5- | S-5- | S-5- |
| (Pattern | | molar ratio counterion soln., stirred | MAPB | MAPB | MAPB |
| 10A) | | at 40° C., in solution after ~6 hrs of | Pattern | Pattern | Pattern |
| | | stirring, opened for evaporation, | 10A | 10A | 10A |
| | | solid after 7 days of evaporation, | | | |
| | | analyzed by PXRD, dried overnight | | | |
| | | at 40° C. in vacuum oven, analyzed | | | |
| | | by PXRD, DSC, and TGA, | | | |
| | | analyzed by optical microscopy, | | | |
| | | ~10 mgs staged at 40° C./75% RH, | | | |
| | | pulled from staging after 1 day, | | | |
| | | analyzed by PXRD | | | |

### EXAMPLE 22. Solubility Assessment in FaSSIF Media

The S-5-MAPB Patterns 1A, 2B and 10A scale up samples were tested for their approximate solubility in FaSSIF V2 media as shown below in Table 18. All three samples were found to have solubility of >10 mg/mL and remained in solution after overnight stirring.

TABLE 18
Solubility Assessment in FaSSIF Media of Patterns 1A, 2B and 10A

| Procedure/Comments | Solubility | PXRD Pattern |
|---|---|---|
| 10 mg of Pattern 1A + FaSSIF V2 media added in 1 mL steps | >10 mg/mL | N/A (in |
| at a rate of 1 mL/5 min until dissolved, stirred at room temp, | | solution) |
| dissolved after 1 mL, left stirring overnight, remained in | | |
| solution after overnight stirring, terminated | | |
| 10 mg of Pattern 2B + FaSSIF V2 media added in 1 mL steps | >10 mg/mL | N/A (in |
| at a rate of 1 mL/5 min until dissolved, stirred at room temp, | | solution) |
| dissolved after 1 mL, left stirring overnight, remained in | | |
| solution after overnight stirring, terminated | | |
| 10 mg of Pattern 10A + FaSSIF V2 media added in 1 mL | >10 mg/mL | N/A (in |
| steps at a rate of 1 mL/5 min until dissolved, stirred at room | | solution) |
| temp, dissolved after 1 mL, left stirring overnight, remained | | |
| in solution after overnight stirring, terminated | | |

### EXAMPLE 23. Scale-Up and Stability Study

Scale up study of selected salts (S-5-MAPB Patterns 1A, 4A, and 8A) to −250 mg completed. Patterns 1A, 4A, and 8A were all scaled up successfully. All three samples were then tested for their solid-state stability as shown below in Table 19. (API=S-5-MAPB Pure Enantiomer)

TABLE 19
Scale-up and stability study Enantiomer Pattern 1A, 4A, 8A

| | | | | | | PXRD |
|---|---|---|---|---|---|---|
| | | | | | | Pattern |
| | | | PXRD | PXRD | PXRD | (40° C./ |
| | | | Pattern | Pattern (2 | Pattern | 75% |
| Counter | | | (overnight | days of | (Vacuum | RH |
| ion | Solvent | Procedure/Comments | stirring) | stirring) | Dried) | sample) |
| HCl | THF | 240 mg API + 10 vols. | S-5- | S-5- | S-5- | S-5- |
| (Pattern | | solvent + 1:1 molar ratio | MAPB | MAPB | MAPB | MAPB |
| 1A) | | counterion soln., stirred | Pattern | Pattern | Pattern | Pattern |
| | | at 40° C., slurry after | 1A | 1A | 1A | 1A |
| | | overnight stirring, | | | | |
| | | sampled (.5 mL) and | | | | |
| | | vacuum filtered for 30 | | | | |
| | | min, analyzed by PXRD, | | | | |
| | | cont stirring, slurry after | | | | |
| | | 2 days of stirring, | | | | |
| | | vacuum filtered for | | | | |
| | | 30 min, analyzed by | | | | |
| | | PXRD, dried overnight at | | | | |
| | | 40° C. in vacuum oven, | | | | |
| | | analyzed by optical | | | | |
| | | microscopy, PXRD, DSC | | | | |
| | | and TGA, ~10 mgs staged | | | | |
| | | at 40° C./75% RH, pulled | | | | |
| | | from staging after 1 day, | | | | |
| | | analyzed by PXRD | | | | |
| H₃PO₄ | Acetone | 250 mg API + 10 vols. | S-5- | S-5- | S-5- | S-5- |
| (Pattern | | solvent + 1:1 molar ratio | MAPB | MAPB | MAPB | MAPB |
| 4A) | | counterion soln., stirred | Pattern | Pattern | Pattern | Pattern |
| | | at 40° C., slurry after | 4A | 4A | 4A | 4A |
| | | overnight stirring, | | | | |
| | | sampled (.5 mL) and | | | | |
| | | vacuum filtered for 30 | | | | |
| | | min, analyzed by PXRD, | | | | |
| | | cont stirring, slurry after | | | | |
| | | 2 days of stirring, | | | | |
| | | vacuum filtered for | | | | |
| | | 30 min, analyzed by | | | | |
| | | PXRD, dried overnight at | | | | |
| | | 40° C. in vacuum oven, | | | | |
| | | analyzed by optical | | | | |
| | | microscopy, PXRD, DSC | | | | |
| | | and TGA, ~10 mgs staged | | | | |
| | | at 40° C./75% RH, pulled | | | | |
| | | from staging after 1 day, | | | | |
| | | analyzed by PXRD | | | | |
| Oxalic | EtOH:water | 240 mg API + 10 vols. | N/A | S-5- | S-5- | S-5- |
| (Pattern | 90:10 | solvent + 1:1 molar ratio | | MAPB | MAPB | MAPB |
| 8A) | | counterion soln., stirred | | Pattern | Pattern | Pattern |
| | | at 40° C., in solution after | | 8A | 8A | 8A |
| | | overnight stirring, | | | | |
| | | opened for evaporation, | | | | |
| | | solid after overnight | | | | |
| | | evaporation, analyzed by | | | | |
| | | PXRD, dried overnight at | | | | |
| | | 40° C. in vacuum oven, | | | | |
| | | analyzed by optical | | | | |
| | | microscopy, PXRD, DSC | | | | |
| | | and TGA, ~10 mgs staged | | | | |
| | | at 40° C./75% RH, pulled | | | | |
| | | from staging after 1 day, | | | | |
| | | analyzed by PXRD | | | | |

### EXAMPLE 24. Solubility Assessment in FaSSIF Media

The approximate solubility of Enantiomer Patterns 1A, 4A and 8A scale up samples was measured in FaSSIF V2 media as shown below in Table 20. Enantiomer Patterns 1A and 4A were found to have a solubility >10 mg/mL. Enantiomer Pattern 8A was found to have a solubility between 10 mg/mL and 5 mg/mL. All three samples remained in solution after overnight stirring.

TABLE 20
Solubility Assessment in FaSSIF Media of Patterns 1A, 4A and 8A

| | Solubility | |
|---|---|---|
| Procedure/Comments | mg/mL | PXRD Pattern |
| 10 mg of S-5-MAPB Pattern 1A + FaSSIF V2 media | >10 | N/A (in solution) |
| added in 1 mL steps at a rate of 1 mL/5 min until | | |
| dissolved, stirred at room temp, dissolved after 1 mL, | | |
| remained in solution after overnight stirring, terminated | | |
| 10 mg of S-5-MAPB Pattern 4A + FaSSIF V2 media | >10 | N/A (in solution) |
| added in 1 mL steps at a rate of 1 mL/5 min until | | |
| dissolved, stirred at room temp, dissolved after 1 mL, | | |
| remained in solution after overnight stirring, terminated | | |
| 10 mg of S-5-MAPB Pattern 8A + FaSSIF V2 media | 5 < S < 10 | N/A (in solution) |
| added in 1 mL steps at a rate of 1 mL/5 min until | | |
| dissolved, stirred at room temp, dissolved after 2 mL, | | |
| remained in solution after overnight stirring, terminated | | |

### EXAMPLE 25. R-5-MAPB Freebase Isolation/Liquid-Liquid Extraction

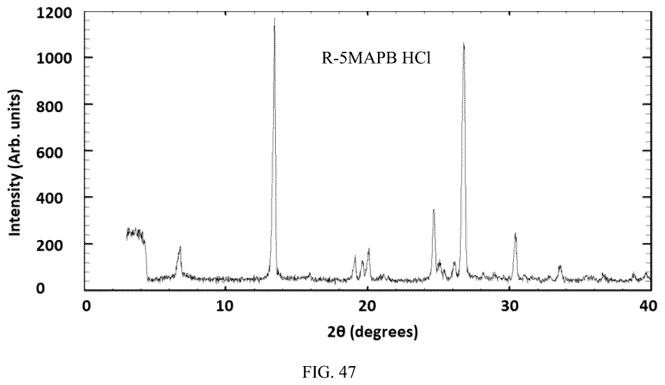

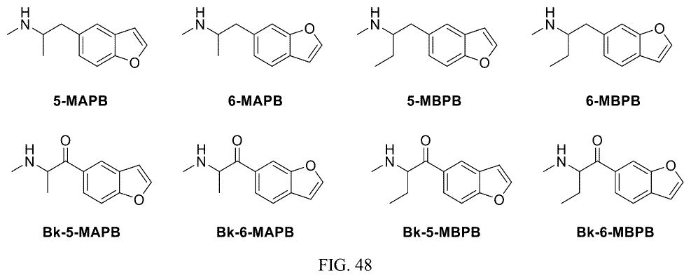

Liquid-Liquid Extraction (LLE) was used to isolate R-5-MAPB freebase from R-5-MAPB hydrochloride (R5-MAPB HCl) using the conditions in Table 21. FIG. 47 is the XRPD pattern for R-5-MAPB HCl. This XRPD pattern is referred to as R-Enantiomer Pattern 1A (for 300 mg of 5R-MAPB HCl, 1 vol. solvent is equivalent to 300 μL).

TABLE 21
R-5-MAPB Freebase from R-5-MAPB HCl

| | Solvent | |
|---|---|---|
| Counterion | (Density) | Procedure/Comments |
| NaOH | EtOAc | 300 mg R-5-MAPB HCl + 10 vols. solvent + |
| | (0.902 | NaOH stock soln. in water (1.1:1 molar |
| | g/mL) | ratio) + additional water (10 vols. water total) |

### EXAMPLE 26. Salt Screening Experiments of R-5-MAPB Pure Enantiomer

Salt screening experiments of R-5-MAPB pure enantiomer were conducted as shown below in Table 22. All crystalline salts afford Pattern 1A, which was the same pattern observed from the R-5-MAPB HCl used as the starting material in Example 25 (for 27 mg of R-5-MAPB, 1 vol. solvent=27 μL).

TABLE 22
Salt screening experiments of R-5-MAPB Pure Enantiomer

| No. | Counterion | Solvent | Procedure/Comments | PXRD Result |
|---|---|---|---|---|
| 1 | HCl | Acetone | 27 mg R-5-MAPB + 10 vols. solvent + 1:1 | R-5-MAPB |
| | | | molar ratio counterion soln., stirred at 40° | HCl Pattern 1A |
| | | | C., slurry after overnight stirring, | |
| | | | centrifuged to harvest solids, analyzed by | |
| | | | PXRD | |
| 2 | HCl | MeOH: | 27 mg R-5-MAPB + 10 vols. solvent + 1:1 | R-5-MAPB |
| | | water | molar ratio counterion soln., stirred at 40° | HCl Pattern 1A |
| | | 90:10 | C., in solution after overnight stirring, | |
| | | | opened for evaporation, solid after | |
| | | | overnight evaporation, analyzed by PXRD | |
| 3 | HCl | THF | 27 mg R-5-MAPB + 10 vols. solvent + 1:1 | R-5-MAPB |
| | | | molar ratio counterion soln., stirred at 40° | HCl Pattern 1A |
| | | | C., slurry after overnight stirring, | |
| | | | centrifuged to harvest solids, analyzed by | |
| | | | PXRD | |
| 4 | HCl | EtOH: | 27 mg R-5-MAPB + 10 vols. solvent + 1:1 | R-5-MAPB |
| | | water | molar ratio counterion soln., stirred at 40° | HCl Pattern 1A |
| | | 90:10 | C., in solution after overnight stirring, | |
| | | | opened for evaporation, solid after | |
| | | | overnight evaporation, analyzed by PXRD | |
| 5 | HCl | CAN | 27 mg R-5-MAPB + 10 vols. solvent + 1:1 | R-5-MAPB |
| | | | molar ratio counterion soln., stirred at 40° | HCl Pattern 1A |
| | | | C., solid precipitate with solution after | |
| | | | overnight stirring, solids harvested and | |
| | | | analyzed by PXRD | |

### Example 27. Non-Racemic 5-Mapb Human Monoamine Transporter (Hmat) Release Assays

Based on results from the marble burying assay that non-racemic mixtures of 5-MAPB enantiomers had non-additive effects, further in vitro measures of serotonin and dopamine release were made using cells that expressed human monoamine transporters, serotonin (hSERT) and dopamine (hDAT) transporter. Measures of in vitro serotonin and dopamine release using Chinese hamster ovary cells that expressed human serotonin (hSERT) or dopamine (hDAT) transporters were made. These produced surprising findings where non-racemic mixtures of 5-MAPB produced lower DAT to SERT ratios than the S-enantiomer or the racemate. This surprising finding suggests non-racemic mixtures may have lessened abuse liability compared to the S-enantiomer or the racemate. These findings could not be predicted from the activity of the individual enantiomers or the racemate.

TABLE 23
Effects of 5-MAPB on DAT and SERT

| | EC₅₀ DAT | EC₅₀ SERT | |
|---|---|---|---|
| | (Mean ± | (Mean ± | DAT/SERT |
| | SEM, nM) | SEM, nM) | ratio* |
| S-5-MAPB | 258 ± 99 | 67 ± 15 | 0.26 |
| 75% S-5- | 632 ± 113 | 80 ± 7 | 0.13 |
| MAPB | | | |
| RS-5-MAPB | 459 ± 48 | 90 ± 16 | 0.20 |
| 75% R-5- | 794 ± 182 | 122 ± 13 | 0.15 |
| MAPB | | | |
| R-5-MAPB | 1951 ± 401 | 184 ± 3 | 0.09 |

*DAT/SERT ratios are calculated here as (DAT EC₅₀)⁻¹/(SERT EC₅₀)⁻¹ where larger number indicates higher DAT selectivity

These data indicate that mixtures of enantiomers other than racemic produce lower DAT/SERT ratios than the simple racemic mixture. This could be the result of interactions between the reuptake inhibiting and release inducing properties of the individual enantiomers.

hSERT Release Measurement Methods

Chinese hamster ovary cells expressing human SERT were seeded in Cytostar™ (PerkinElmer) plate with standard culture medium the day before the experiment at a single density (5000 cells/assay). Cells were incubated overnight with 5% CO₂ at 37° C. The day of experiment, the medium was replaced by incubation buffer (140mMNaCl, 4.8 mM KCl, 1.2 mM MgSO₄, 0.1 mM KH₂PO₄, 10 mM HEPES, pH 7.4) with a single concentration of [³H] Serotonin at 150 nM. Experiments comparing release in radioligand-free incubation buffer versus incubation buffer containing [³H] Serotonin determined that the latter provided better signal stability. Therefore, this was used for experiments.

In control wells, the specificity of hSERT uptake was verified by adding the reference control imipramine (100 μM).

Two control conditions were used: (1) buffer only (with 1% DMSO concentration to match that in the test compound condition) to verify the background level of release; and (2) one reference SERT substrate compound, norfenfluramine, at 100 μM, to make it possible to calculate a relative Emax. Pilot studies varying DMSO concentration from 0.1 to 3% indicated that signal decreased at higher DMSO concentrations but that 1% DMSO retained good properties.

Cells were incubated at room temperature at different incubation times and radioactivity counted. Test compounds were measured at concentrations of 1e-10, 1e-09, 1e-08, 1e-07, 1e-06, 1e-05, and 1e-04 M. Each experiment was performed in duplicate (n=2) and results calculated at two inhibition times (60 and 90).

hDAT Release Measurement Methods

Chinese hamster ovary cells expressing human DAT were seeded in Cytostar™ plate with standard culture medium the day before experiment at one single density (2500 cells/assay). Cells were incubated overnight with 5% CO₂ at 37° C. The day of experiment, the medium was replaced by incubation buffer (TrisHCl 5 mM, 120 mM NaCl, 5.4 mM KCl, 1.2 mM MgSO₄,1.2 mM CaCl₂, Glucose 5 mM, 7.5 mM HEPES, pH 7.4) with a single concentration of [3H]dopamine at 300 nM. Experiments comparing release in radioligand-free incubation buffer versus incubation buffer containing [³H]dopamine determined that the latter provided better signal stability. Therefore, this was used for experiments.

In control wells, the specificity of DAT uptake was verified by adding the reference control GBR 12909 (10 μM).

For all assays, three reference conditions were employed: (1) radioligand-containing buffer only, to verify the control level of release, (2) buffer with 1% DMSO (solvent used to solubilize the test compounds), (3) 100 μM amphetamine (in 1% DMSO) to make it possible to calculate a relative Emax.

Cells were incubated at room temperature at different incubation times and radioactivity counted. Test compounds were measured at concentrations of 1e-10, 1e-09, 1e-08, 1e-07, 1e-06, 1e-05, and 1e-04 M. Each experiment was performed in duplicate (n=2) and results calculated at two inhibition times (60 and 90).

### Statistical Analysis

EC/IC50s were calculated using the R packages drm (to fit the regression model) and LL.4 (to define the structure of the log-logistic regression model). Values were fit to the following function:

f(x)=c+(d−c)/(1+exp(b(log(x)−log(e)))

where b=the Hill coefficient, c=minimum value, d=maximum value, and e=EC₅₀/IC₅₀.

Values were calculated for both experimental repetitions at both stable inhibition times (60 and 90 minutes), resulting in four estimates for each compound and transporter. These four values were averaged to produce a final estimate for each compound and transporter. Standard errors of the mean were also calculated based on the four values.

### Example 28: Human Effects of Non-Racemic 5-Mapb

The effects of non-racemic 5-MAPB HCl were tested by a healthy human individual at four different ratios of enantiomers plus the racemate as a control:
54 mg S and 0 mg R (100% S)
47 mg S and 7 mg R (about 87% S)
36 mg S and 18 mg R (about 67% S)
27 mg S and 27 mg R (about 50% S)
7 mg S and 47 mg R (about 13% S)

Two trials were carried out at each ratio, except for 13% S where only one was conducted. Trials were at least 72 h apart.

5-MAPB HCl was dissolved in 1.5 ml distilled water and consumed in two halves, separated by 1 hour. Starting at 3 hours after administration, tablets of 250 mg ascorbic acid and capsules of 300 mg alpha lipoic acid were taken ad libitum (approximately every hour for a total of 4 administrations).

Measurements were 0-100 ratings of “good drug effects” (abbreviated as Good), “bad drug effects” (abbreviated as Bad), and “emotional openness” (abbreviated as Open), comparable to the visual analog ratings (e.g., Morean et al. 2013. Psychopharmacology, 227(1), 177-192) and verbal ratings (Mendelson et al. 1996. Clinical Pharmacology & Therapeutics, 60(1), 105-114) that are common in psychopharmacology research. Measurements were made approximately every 2 hours until post 6 hours and the maximum ratings per session were analyzed. Additionally, an index of good drug effects versus emotional openness, calculated as (Open−Good)/(200, the theoretical maximum of Open+ Good), was constructed at each time point and the maximum analyzed. In healthy volunteers, Good Ratings can be considered a predictor of abuse liability. Accordingly, this index can be used as an indicator of the balance of a therapeutic effect (emotional openness) vs abuse liability.

Historic data of two trials 50 mg RS-5-MAPB Cl from the same individual were also included in the analysis for comparison. Methods for these data were similar except that doses were taken as a bolus and the setting was different.

Table 24 below indicates maximums for individual measures and for the open vs good index, averaged from all sessions (N=2, except for 13% S-5-MAPB where N=1). Qualitatively, all conditions produced subtle emotional effects, including decreases in negative affect and increases in stability of mood, without sensory distortion. 100% S appeared to have effects of less duration than conditions that included the R-enantiomer. A key finding was that non-racemic mixtures appeared to have a higher Open vs Good index, suggesting that they were better able to facilitate emotional openness while minimizing relative abuse liability.

TABLE 24
Self-Reported Ratings of Enantiomerically Enriched 5-MAPB

| | | | | Open vs |
|---|---|---|---|---|
| Condition | Good | Bad | Open | Good Index |
| 100% S-5- | 55% | 1% | 65% | 0.17 |
| MAPB | | | | |
| 87% S-5- | 40% | 5% | 65% | 0.20 |
| MAPB | | | | |
| 67% S-5- | 37% | 5% | 55% | 0.21 |
| MAPB | | | | |
| RS-5-MAPB | 50% | 10% | 45% | −0.03 |
| 13% S-5- | 13% | 15% | 15% | 0.05 |
| MAPB | | | | |

### Example 29 Evaluation of Entactogenic Effect of Decreased Neuroticism

The entactogenic effect of decreased neuroticism can be measured as a decrease in social anxiety using the Brief Fear of Negative Evaluation-revised (BFNE) (Carleton et al., 2006, Depression and Anxiety, 23(5), 297-303; Leary, 1983, Personality and Social Psychology bulletin, 9(3), 371-375). This 12-item Likert scale questionnaire measures apprehension and distress due to concerns about being judged disparagingly or with hostility by others. Ratings use a five-point Likert scale with the lowest, middle, and highest values labeled with “much less than normal,” “normal,” and “much more than normal.” The BFNE can be administered before and repeatedly during therapeutic drug effects. Participants are instructed to answer how they have been feeling for the past hour, or otherwise during the effect of the drug. Baseline-subtracted responses are typically used in statistical models.

### Example 30 Evaluation of Entactogenic Effect of Authenticity

The entactogenic effect of authenticity can be measured using the Authenticity Inventory (Kernis & Goldman. 2006. Advances in experimental social psychology, 38, 283-357) as modified by Baggott et al (Journal of Psychopharmacology 2016, 30.4: 378-87). Administration and scoring of the instrument is almost identical to that of the BFNE. The Authenticity Inventory consists of the following items, which are each rated on a 1-5 scale, with select items reverse scored as specified by Kernis & Goldman:
I am confused about my feelings.
I feel that I would pretend to enjoy something when in actuality I really didn't.
For better or worse, I am aware of who I truly am.
I understand why I believe the things I do about myself
I want the people with whom I am close to understand my strengths.
I actively understand which of my self-aspects fit together to form my core or true self.
I am very uncomfortable objectively considering my limitations and shortcomings.
I feel that I would use my silence or head-nodding to convey agreement with someone else's statement or position even though I really disagreed.
I have a very good understanding of why I do the things I do.
I am willing to change myself for others if the reward is desirable enough.
I would find it easy to pretend to be something other than my true self.
I want people with whom I am close to understand my weaknesses.
I find it difficult to critically assess myself. (unchanged)
I am not in touch with my deepest thoughts and feelings.
I feel that I would make it a point to express to those I am close with how much I truly care for them.
I have difficulty accepting my personal faults, so I try to cast them in a more positive way.
I feel that I idealize the people close to me rather than objectively see them as they truly are.
If asked, people I am close to could accurately describe what kind of person I am.
I prefer to ignore my darkest thoughts and feelings
I am aware of times when I am not being my true self.
I am able to distinguish the self-aspects that are important to my core or true self from those that are unimportant.
People close to me would be shocked or surprised if they discovered what I am keeping inside me.
It is important for me to understand the needs and desires of those with whom I am close.
I want people close to me to understand the real me, rather than just my public persona or “image”.
I could act in a manner that is consistent with my personally held values, even if others criticized me or rejected me for doing so.
If a close other and I were in disagreement, I would rather ignore the issue than constructively work it out.
I feel that I would do things that I don't want to do merely to avoid disappointing people.
My behavior expresses my values.
I actively attempt to understand myself as well as possible.
I feel that I'd rather feel good about myself than objectively assess my personal limitations and shortcomings.
My behavior expresses my personal needs and desires.
I have on a “false face” for others to see.
I feel that I would spend a lot of energy pursuing goals that are very important to other people even though they are unimportant to me.
I am not in touch with what is important to me.
I try to block out any unpleasant feelings I have about myself.
I question whether i really know what I want to accomplish in my lifetime.
I am overly critical about myself.
I am in touch with my motives and desires.
I feel that I would deny the validity of any compliments that I receive.
I place a good deal of importance on people close to me understanding who I truly am.
I find it difficult to embrace and feel good about the things I have accomplished.
If someone pointed out or focused on one of my shortcomings, I would quickly try to block it out of my mind and forget it.
The people close to me could count on me being who I am, regardless of what setting we were in.
My openness and honesty in close relationships are extremely important to me.
I am willing to endure negative consequences by expressing my true beliefs about things.

### EXAMPLE 31. Salt Studies of S-6-MAPB in Acetone or MeOH:H₂O

Salt studies of S-6-MAPB were conducted using the conditions in Table 25. The below technique was used to generate the XRPD patterns in FIG. 50, FIG. 51, and FIG. 52 (For 35 mg of S-6-MAPB HCl, 1 vol. solvent=35 μL).

TABLE 25
Salt screening experiments

| | | | PXRD |
|---|---|---|---|
| Counterion | Solvent | Procedure/Comments | Result |
| HCl | Acetone | 35 mg S-6-MAPB HCl + 10 vols. solvent + 1:1 molar | Pattern |
| | | ratio counterion soln., stirred at 40° C., in solution after | 1A |
| | | overnight stirring, opened for evaporation, solid after 3 | |
| | | days of evaporation, analyzed by PXRD, dried | |
| | | overnight at 40° C. in vacuum oven, analyzed by DSC | |
| | | and TGA | |
| HBr | Acetone | 35 mg S-6-MAPB HCl + 10 vols. solvent + 1:1 molar | Pattern |
| | | ratio counterion soln., stirred at 40° C., in solution after | 2A |
| | | overnight stirring, opened for evaporation, solid after 3 | |
| | | days of evaporation, analyzed by PXRD, dried | |
| | | overnight at 40° C. in vacuum oven, analyzed by DSC | |
| | | and TGA | |
| H₃PO₄ | Acetone | 35 mg S-6-MAPB HCl + 10 vols. solvent + 1:1 molar | Pattern |
| | | ratio counterion soln., stirred at 40° C., slurry after | 3A |
| | | overnight stirring, centrifuged to harvest solids, | |
| | | analyzed by PXRD, dried overnight at 40° C. in vacuum | |
| | | oven, analyzed by DSC and TGA | |
| Succinic | Acetone | 35 mg S-6-MAPB HCl + 10 vols. solvent + 1:1 molar | N/A |
| | | ratio counterion soln., stirred at 40° C., in solution after | (gel) |
| | | overnight stirring, opened for evaporation, gel after 3 | |
| | | days of evaporation, terminated | |
| Oxalic | Acetone | 35 mg S-6-MAPB HCl + 10 vols. solvent + 1:1 molar | Pattern |
| | | ratio counterion soln., stirred at 40° C., in solution after | 5A |
| | | overnight stirring, opened for evaporation, solid after 3 | |
| | | days of evaporation, analyzed by PXRD, dried | |
| | | overnight at 40° C. in vacuum oven, analyzed by DSC | |
| | | and TGA | |
| Maleic | Acetone | 35 mg S-6-MAPB HCl + 10 vols. solvent + 1:1 molar | N/A |
| | | ratio counterion soln., stirred at 40° C., in solution after | (gel) |
| | | overnight stirring, opened for evaporation, gel after 3 | |
| | | days of evaporation, terminated | |
| Fumaric | Acetone | 35 mg S-6-MAPB HCl + 10 vols. solvent + 1:1 molar | N/A |
| | | ratio counterion soln., stirred at 40° C., in solution after | (gel) |
| | | overnight stirring, opened for evaporation, gel after 3 | |
| | | days of evaporation, terminated | |
| HCl | MeOH: | 35 mg S-6-MAPB HCl + 10 vols. solvent + 1:1 molar | Pattern |
| | water | ratio counterion soln., stirred at 40° C., in solution after | 1A |
| | 90:10 | overnight stirring, opened for evaporation, solid after 3 | |
| | | days of evaporation, analyzed by PXRD, dried | |
| | | overnight at 40° C. in vacuum oven | |
| HBr | MeOH: | 35 mg S-6-MAPB HCl + 10 vols. solvent + 1:1 molar | Pattern |
| | water | ratio counterion soln., stirred at 40° C., in solution after | 2A |
| | 90:10 | overnight stirring, opened for evaporation, solid after 3 | |
| | | days of evaporation, analyzed by PXRD, dried | |
| | | overnight at 40° C. in vacuum oven | |
| H₃PO₄ | MeOH: | 35 mg S-6-MAPB HCl + 10 vols. solvent + 1:1 molar | Pattern |
| | water | ratio counterion soln., stirred at 40° C., in solution after | 3B |
| | 90:10 | overnight stirring, opened for evaporation, solid after 3 | |
| | | days of evaporation, analyzed by PXRD, dried | |
| | | overnight at 40° C. in vacuum oven, analyzed by DSC | |
| | | and TGA | |
| Succinic | MeOH: | 35 mg S-6-MAPB HCl + 10 vols. solvent + 1:1 molar | N/A |
| | water | ratio counterion soln., stirred at 40° C., in solution after | (gel) |
| | 90:10 | overnight stirring, opened for evaporation, gel after 3 | |
| | | days of evaporation, terminated | |
| Oxalic | MeOH: | 35 mg S-6-MAPB HCl + 10 vols. solvent + 1:1 molar | Pattern |
| | water | ratio counterion soln., stirred at 40° C., in solution after | 5A |
| | 90:10 | overnight stirring, opened for evaporation, solid after 3 | |
| | | days of evaporation, analyzed by PXRD, dried | |
| | | overnight at 40° C. in vacuum oven, analyzed by DSC | |
| | | and TGA | |
| Maleic | MeOH: | 35 mg S-6-MAPB HCl + 10 vols. solvent + 1:1 molar | N/A |
| | water | ratio counterion soln., stirred at 40° C., in solution after | (gel) |
| | 90:10 | overnight stirring, opened for evaporation, gel after 3 | |
| | | days of evaporation, terminated | |
| Fumaric | MeOH: | 35 mg S-6-MAPB HCl + 10 vols. solvent + 1:1 molar | N/A |
| | water | ratio counterion soln., stirred at 40° C., in solution after | (gel) |
| | 90:10 | overnight stirring, opened for evaporation, gel after 3 | |
| | | days of evaporation, terminated | |

### EXAMPLE 32. Salt Studies of S-6-MAPB in THF or EtOH:H₂O

Salt studies of S-6-MAPB were conducted using the conditions in Table 26. The below technique was used to generate the XRPD patterns in FIG. 53 and FIG. 54 (For 35 mg of S-6-MAPB HCl, 1 vol. solvent=35 μL).

TABLE 26
Salt screening experiments

| | | | PXRD |
|---|---|---|---|
| Counterion | Solvent | Procedure/Comments | Result |
| HCl | THF | 35 mg S-6-MAPB + 10 vols. solvent + 1:1 molar ratio | S-6- |
| | | counterion soln., stirred at 40° C., in solution after | MAPB |
| | | overnight stirring, opened for evaporation, solid after | Pattern |
| | | overnight evaporation, analyzed by PXRD, dried | 1A |
| | | overnight at 40° C. in vacuum oven, analyzed by DSC | |
| HBr | THF | 35 mg S-6-MAPB + 10 vols. solvent + 1:1 molar ratio | S-6- |
| | | counterion soln., stirred at 40° C., in solution after | MAPB |
| | | overnight stirring, opened for evaporation, solid after | Pattern |
| | | overnight evaporation, analyzed by PXRD, dried | 2A |
| | | overnight at 40° C. in vacuum oven, analyzed by DSC | |
| | | and TGA | |
| H₃PO₄ | THF | 35 mg S-6-MAPB + 10 vols. solvent + 1:1 molar ratio | N/A |
| | | counterion soln., stirred at 40° C., in solution after | (gel) |
| | | overnight stirring, opened for evaporation, gel after | |
| | | overnight evaporation, terminated | |
| Succinic | THF | 35 mg S-6-MAPB + 10 vols. solvent + 1:1 molar ratio | N/A |
| | | counterion soln., stirred at 40° C., in solution after | (gel) |
| | | overnight stirring, opened for evaporation, gel after 4 | |
| | | days of evaporation, terminated | |
| Oxalic | THF | 35 mg S-6-MAPB + 10 vols. solvent + 1:1 molar ratio | S-6- |
| | | counterion soln., stirred at 40° C., in solution after | MAPB |
| | | overnight stirring, opened for evaporation, solid after 4 | Pattern |
| | | days of evaporation, analyzed by PXRD, dried | 5A |
| | | overnight at 40° C. in vacuum oven | |
| Maleic | THF | 35 mg S-6-MAPB + 10 vols. solvent + 1:1 molar ratio | N/A |
| | | counterion soln., stirred at 40° C., in solution after | (gel) |
| | | overnight stirring, opened for evaporation, gel after 4 | |
| | | days of evaporation, terminated | |
| Fumaric | THF | 35 mg S-6-MAPB + 10 vols. solvent + 1:1 molar ratio | N/A |
| | | counterion soln., stirred at 40° C., in solution after | (gel) |
| | | overnight stirring, opened for evaporation, gel after | |
| | | overnight evaporation, terminated | |
| HCl | EtOH: | 35 mg S-6-MAPB + 10 vols. solvent + 1:1 molar ratio | S-6- |
| | water | counterion soln., stirred at 40° C., in solution after | MAPB |
| | 90:10 | overnight stirring, opened for evaporation, solid after | Pattern |
| | | overnight evaporation, analyzed by PXRD, dried | 1A |
| | | overnight at 40° C. in vacuum oven | |
| HBr | EtOH: | 35 mg S-6-MAPB + 10 vols. solvent + 1:1 molar ratio | S-6- |
| | water | counterion soln., stirred at 40° C., in solution after | MAPB |
| | 90:10 | overnight stirring, opened for evaporation, solid after | Pattern |
| | | overnight evaporation, analyzed by PXRD, dried | 2A |
| | | overnight at 40° C. in vacuum oven, analyzed by DSC | |
| | | and TGA | |
| H₃PO₄ | EtOH: | 35 mg S-6-MAPB + 10 vols. solvent + 1:1 molar ratio | S-6- |
| | water | counterion soln., stirred at 40° C., in solution after | MAPB |
| | 90:10 | overnight stirring, opened for evaporation, solid after | Pattern |
| | | overnight evaporation, analyzed by PXRD, dried | 3A |
| | | overnight at 40° C. in vacuum oven, analyzed by DSC | |
| | | and TGA | |
| Succinic | EtOH: | 35 mg S-6-MAPB + 10 vols. solvent + 1:1 molar ratio | N/A |
| | water | counterion soln., stirred at 40° C., in solution after | (gel) |
| | 90:10 | overnight stirring, opened for evaporation, gel after 4 | |
| | | days of evaporation, terminated | |
| Oxalic | EtOH: | 35 mg S-6-MAPB + 10 vols. solvent + 1:1 molar ratio | S-6- |
| | water | counterion soln., stirred at 40° C., in solution after | MAPB |
| | 90:10 | overnight stirring, opened for evaporation solid after 4 | Pattern |
| | | days of evaporation, analyzed by PXRD, dried | 5A |
| | | overnight at 40° C. in vacuum oven, analyzed by DSC | |
| Maleic | EtOH: | 35 mg S-6-MAPB + 10 vols. solvent + 1:1 molar ratio | N/A |
| | water | counterion soln., stirred at 40° C., in solution after | (gel) |
| | 90:10 | overnight stirring, opened for evaporation, gel after 4 | |
| | | days of evaporation, terminated | |
| Fumaric | EtOH: | 35 mg S-6-MAPB + 10 vols. solvent + 1:1 molar ratio | N/A |
| | water | counterion soln., stirred at 40° C., in solution after | (gel) |
| | 90:10 | overnight stirring, opened for evaporation, gel after | |
| | | overnight evaporation, terminated | |

### EXAMPLE 33. Salt Studies of S-6-MAPB in Acetonitrile (ACN)

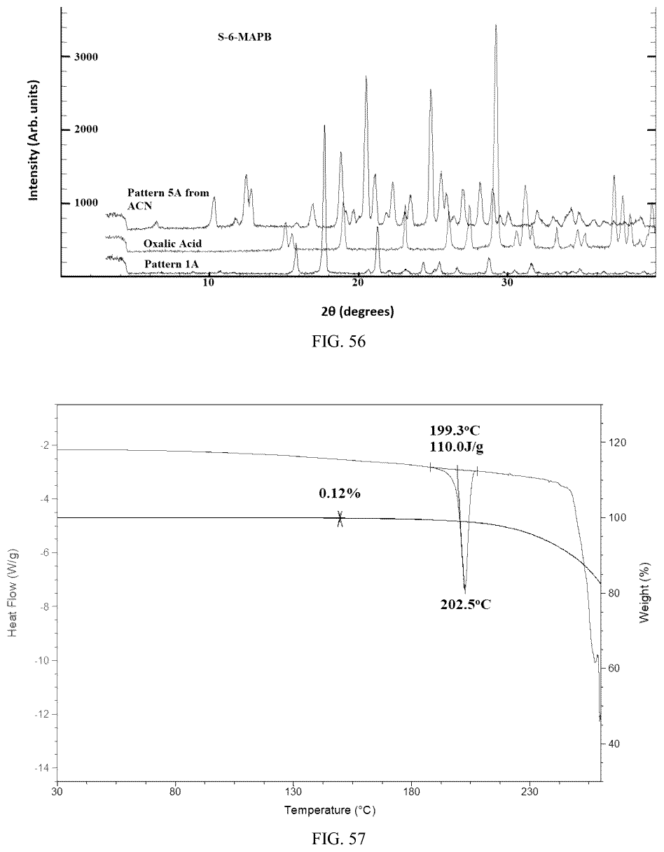

Salt studies of S-6-MAPB were conducted using the conditions in Table 27. The below technique was used to generate the XRPD patterns in FIG. 55 and FIG. 56 (For 35 mg of S-6-MAPB HCl, 1 vol. solvent=35 μL).

TABLE 27
Salt screening experiments

| | | | PXRD |
|---|---|---|---|
| Counterion | Solvent | Procedure/Comments | Result |
| HCl | ACN | 35 mg S-6-MAPB + 10 vols. solvent + 1:1 molar ratio | S-6- |
| | | counterion soln., stirred at 40° C., in solution after | MAPB |
| | | overnight stirring, opened for evaporation, solid after | Pattern |
| | | overnight evaporation, analyzed by PXRD | 1A |
| HBr | ACN | 35 mg S-6-MAPB + 10 vols. solvent + 1:1 molar ratio | S-6- |
| | | counterion soln., stirred at 40° C., in solution after | MAPB |
| | | overnight stirring, opened for evaporation, solid after | Pattern |
| | | overnight evaporation, analyzed by PXRD | 2A |
| H₃PO₄ | ACN | 35 mg S-6-MAPB + 10 vols. solvent + 1:0.33 molar | N/A |
| | | ratio counterion soln. (0.33 eq. counterion), stirred at 40° | (gel) |
| | | C., thin slurry after overnight stirring, centrifuged to | |
| | | harvest solids, deliquesced during PXRD sample prep, | |
| | | terminated | |
| H₃PO₄ | ACN | 35 mg S-6-MAPB + 10 vols. solvent + 1:0.5 molar ratio | N/A |
| | | counterion soln. (0.5 eq. counterion), stirred at 40° C., | (gel) |
| | | thin slurry after overnight stirring, centrifuged to harvest | |
| | | solids, deliquesced during PXRD sample prep, | |
| | | terminated | |
| H₃PO₄ | ACN | 35 mg S-6-MAPB + 10 vols. solvent + 1:1 molar ratio | S-6- |
| | | counterion soln., stirred at 40° C., in solution after | MAPB |
| | | overnight stirring, opened for evaporation, solid after | Pattern |
| | | overnight evaporation, analyzed by PXRD, dried | 3A |
| | | overnight at 40° C. in vacuum oven, analyzed by DSC | |
| Succinic | ACN | 35 mg S-6-MAPB + 10 vols. solvent + 1:1 molar ratio | N/A |
| | | counterion soln., stirred at 40° C., in solution after | (gel) |
| | | overnight stirring, opened for evaporation, gel after 5 | |
| | | days of evaporation, terminated | |
| Oxalic | ACN | 35 mg S-6-MAPB + 10 vols. solvent + 1:1 molar ratio | S-6- |
| | | counterion soln., stirred at 40° C., in solution after | MAPB |
| | | overnight stirring, opened for evaporation, solid after 2 | Pattern |
| | | days of evaporation, analyzed by PXRD | 5A |
| Maleic | ACN | 35 mg S-6-MAPB + 10 vols. solvent + 1:1 molar ratio | N/A |
| | | counterion soln., stirred at 40° C., in solution after | (gel) |
| | | overnight stirring, opened for evaporation, gel after 5 | |
| | | days of evaporation, terminated | |
| Fumaric | ACN | 35 mg S-6-MAPB + 10 vols. solvent + 1:1 molar ratio | N/A |
| | | counterion soln., stirred at 40° C., in solution after | (gel) |
| | | overnight stirring, opened for evaporation, gel after 5 | |
| | | days of evaporation, terminated | |

### EXAMPLE 34: S-BK-5-MAPB Freebase Conversion/Liquid-Liquid Extraction

Liquid-Liquid Extraction (LLE) was used to isolate S-BK-5-MAPB Freebase from Pattern 1A (S-BK-5-MAPB HCl, Pure Enantiomer, FIG. 62) using the conditions shown in Table 28 (for 50 mg of S-BK-MAPB HCl Pure Enantiomer, 1 vol. solvent is equivalent to 50 μL). Table 29 describes liquid-liquid extraction conditions of S-BK-S-MAPB.

TABLE 28
S-BK-5-MAPB Freebase conversion

| Base | Procedure/Comments |
|---|---|
| NaOH | 50 mg API (as HCl salt) + base stock soln. in |
| | water (1.1:1 molar ratio), stirred at RT, brown |
| | gel-like solids unsuspended in solvent, |
| | pipetted out solvent, left vial open at 40° C. to |
| | dry, brown gel after overnight drying |

TABLE 29
Liquid-liquid extraction of S-BK-5-MAPB

| Base | Solvent (Density) | Procedure/Comments |
|---|---|---|
| NaOH | EtOAc (0.902 g/mL) | 50 mg API (as HCl salt) + 18.4 vols. |
| | | solvent + base stock soln. in water |
| | | (1.1:1 molar ratio), vigorously |
| | | shaken and allowed to separate, |
| | | pipetted out top organic layer, |
| | | allowed organic layer to evaporate |
| | | at 40° C., resulting brown gel |
| | | weighed ~33.6 mg after overnight |
| | | evaporation, analyzed by NMR |

### EXAMPLE 35. Scale Up of S-BK-5-MAPB Liquid-Liquid Extraction

Scale ups of the NB118A-71-3 liquid-liquid extraction expt. were performed to isolate large amounts of the Freebase API from the S-BK-5-MAPB HCl salt. ˜1 g API (as HCl salt) plus 18.4 vols. (18.4 mL) of EtOAc were added to NaOH soln. in water (10 mg/mL or 5 mg/mL) for 1.1:1 base:salt molar ratio. Organic phase was then recovered, the solvent was allowed to evaporate at RT, and the resulting brown gel was analyzed by NMR. Table 30 describes scale up conditions of liquid-liquid extraction conditions of S-BK-5-MAPB.

TABLE 30
Liquid-liquid extraction scale up experiments of S-BK-5-MAPB

| | Solvent | |
|---|---|---|
| Base | (Density) | Procedure/Comments |
| NaOH | EtOAc | 1 g API (as HCl salt) + 18.4 vols. solvent + base stock soln. in water (1.1:1 |
| | (0.902 g/mL) | molar ratio), vigorously shaken and allowed to separate |
| NaOH | EtOAc | 1 g API (as HCl salt) + 18.4 vols. solvent + base stock soln. in water (1.1:1 |
| | (0.902 g/mL) | molar ratio), vigorously shaken and allowed to separate |
| NaOH | EtOAc | The top organic layers of S-BK-5-MAPB were pipetted out and combined, |
| | (0.902 g/mL) | concentration calculated as ~48.4 mg/mL, analyzed by NMR |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | The top organic layers of S-BK-5-MAPB were pipetted out and combined, |
| | (0.902 g/mL) | concentration calculated as ~23.4 mg/mL, analyzed by NMR |

### EXAMPLE 36 Salt Studies of S-BK-5-MAPB Pure Enantiomer in Acetone

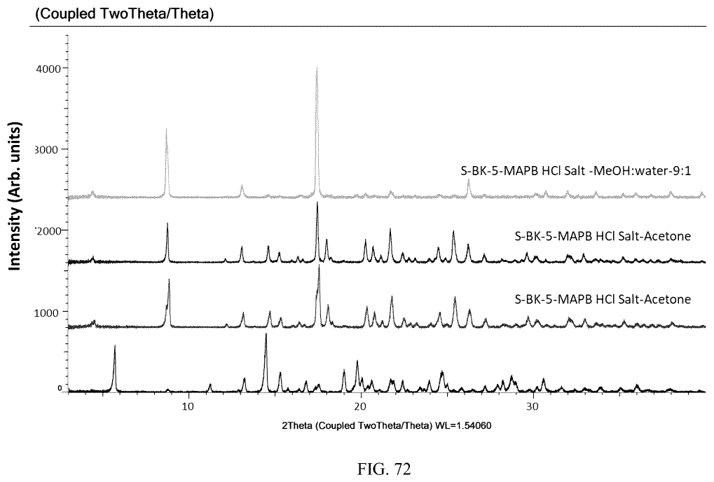

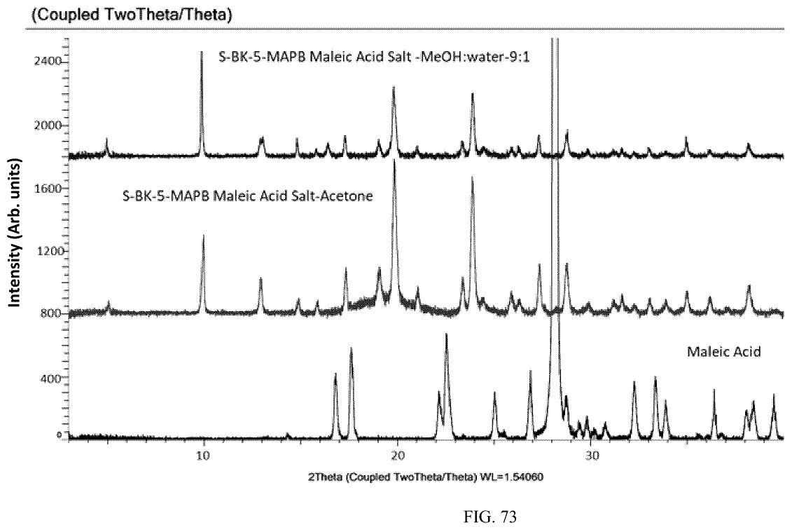

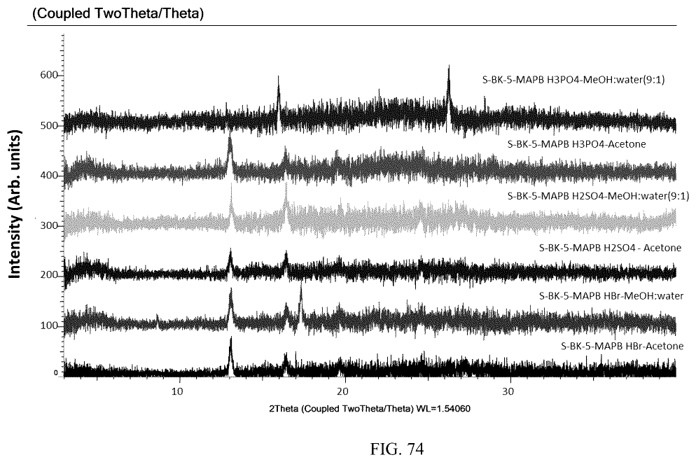

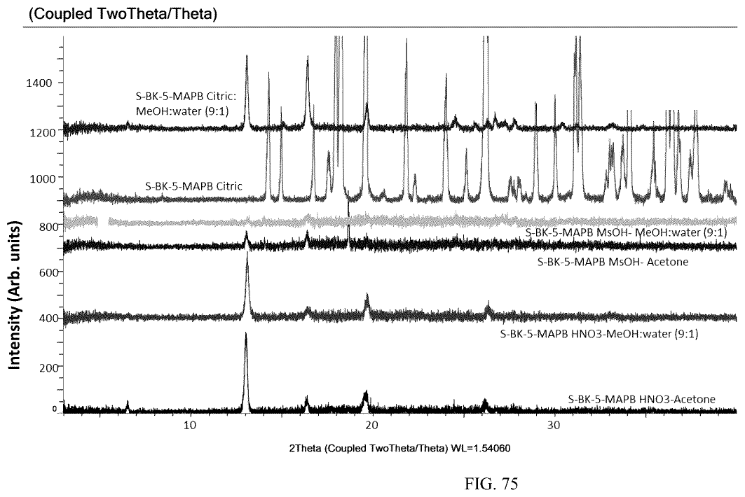

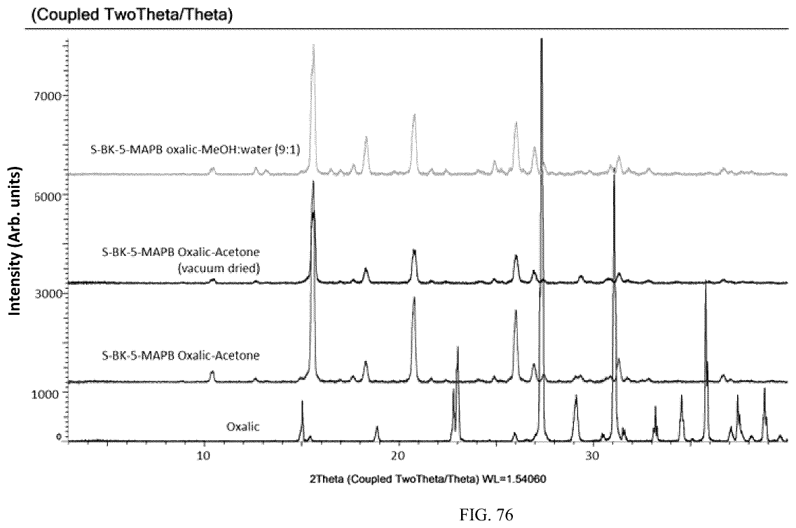

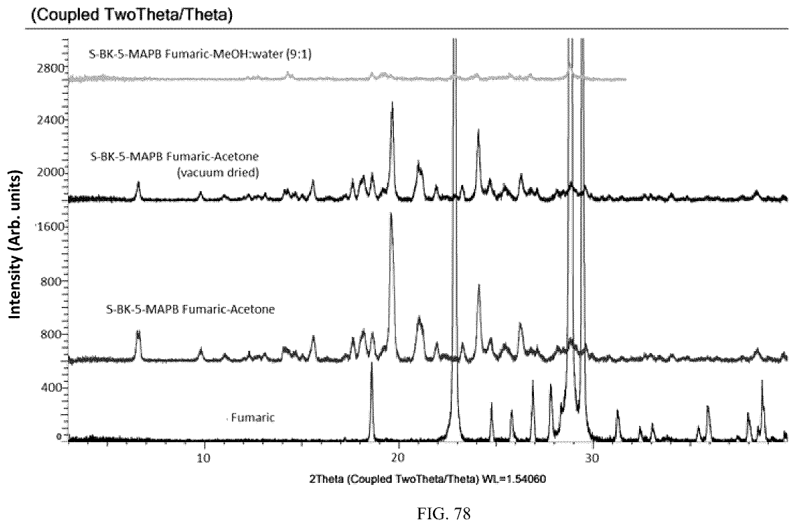

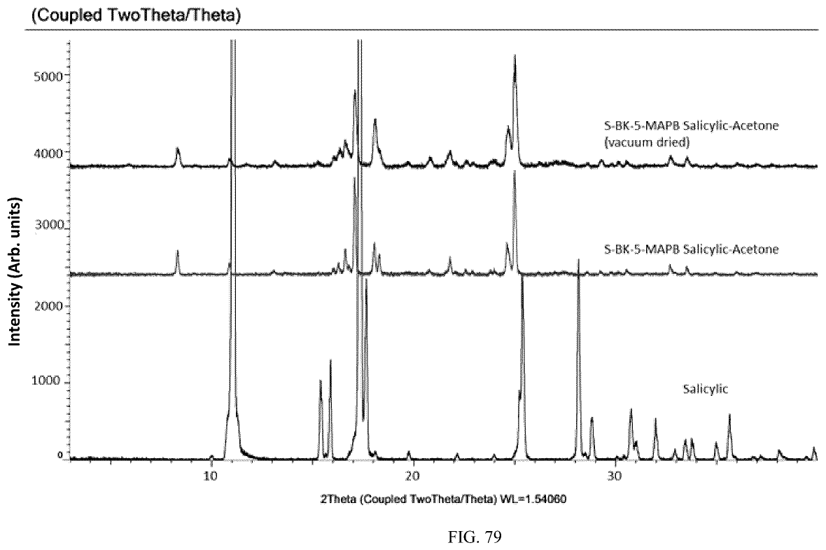

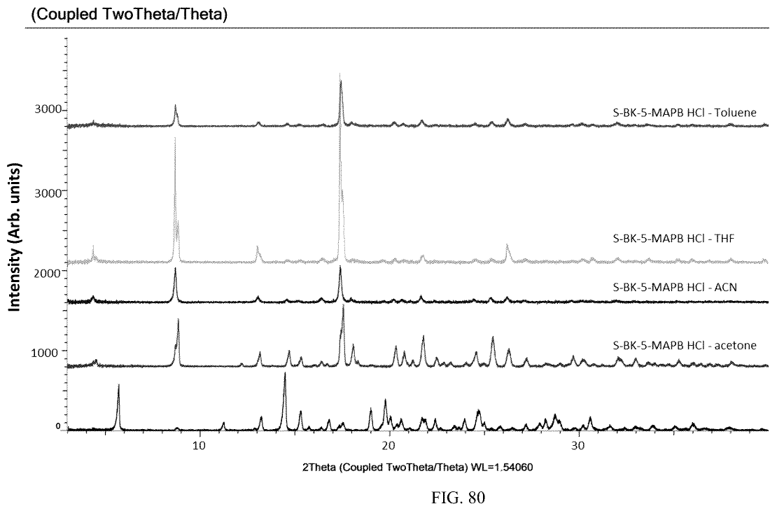

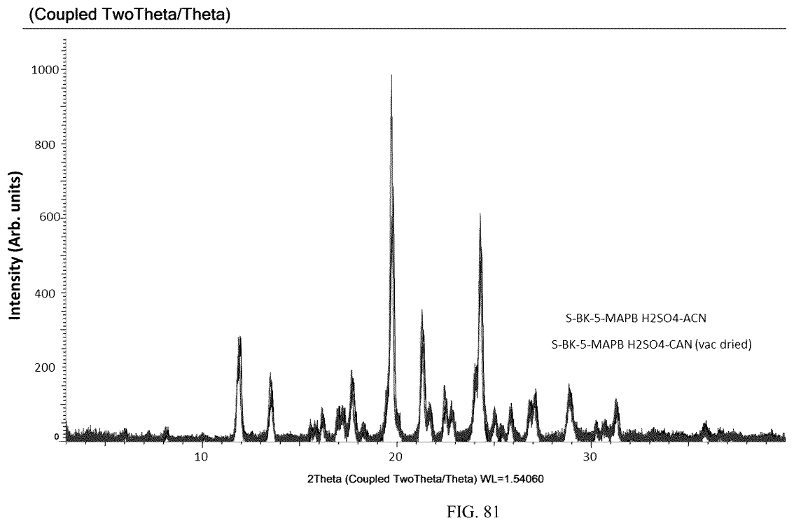

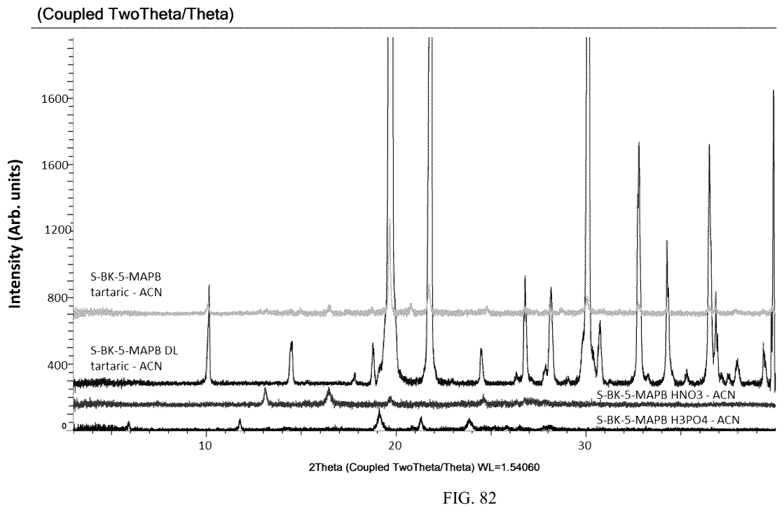

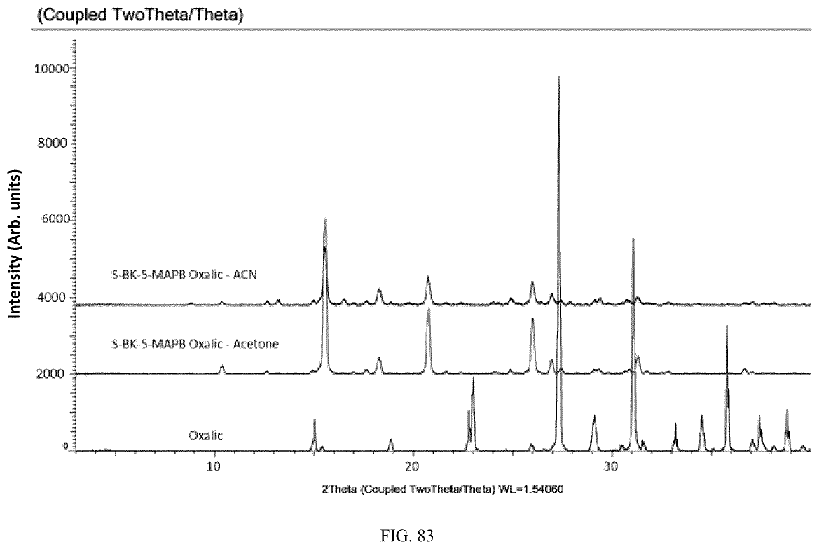

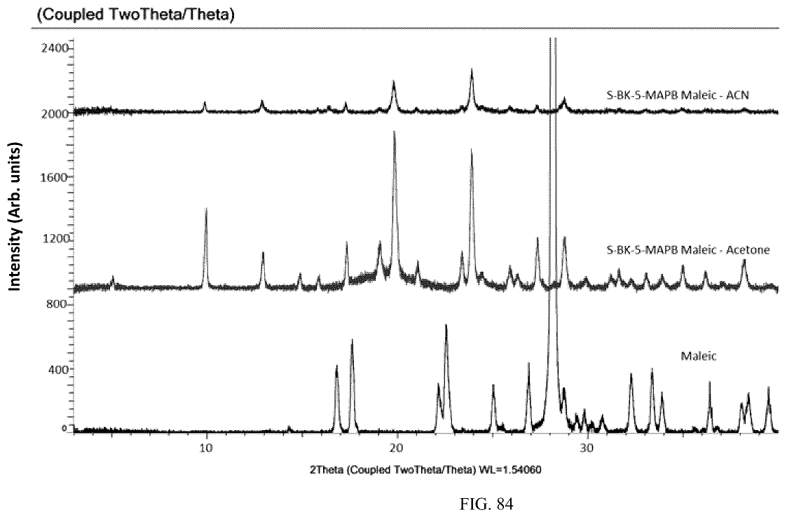

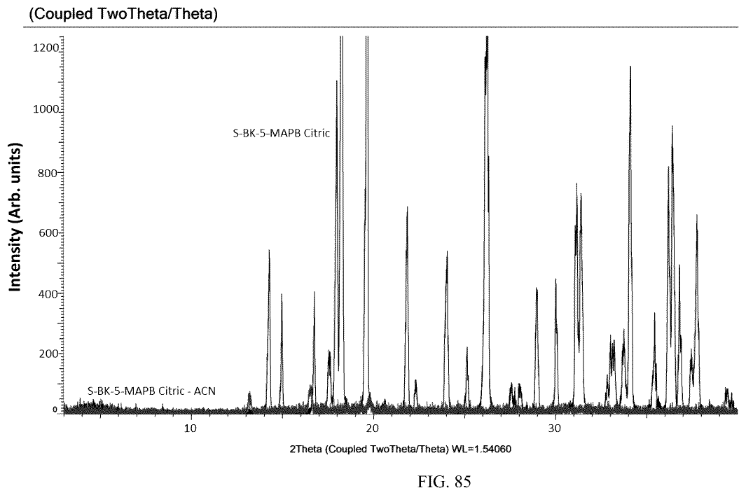

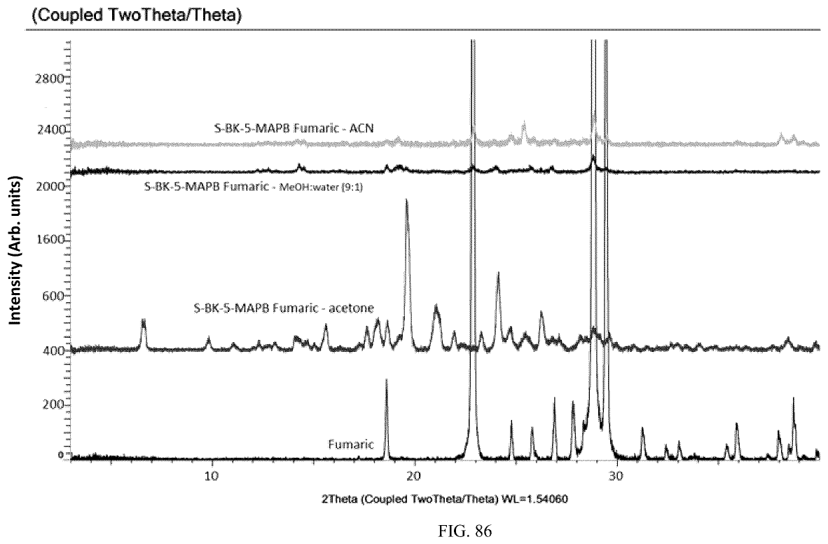

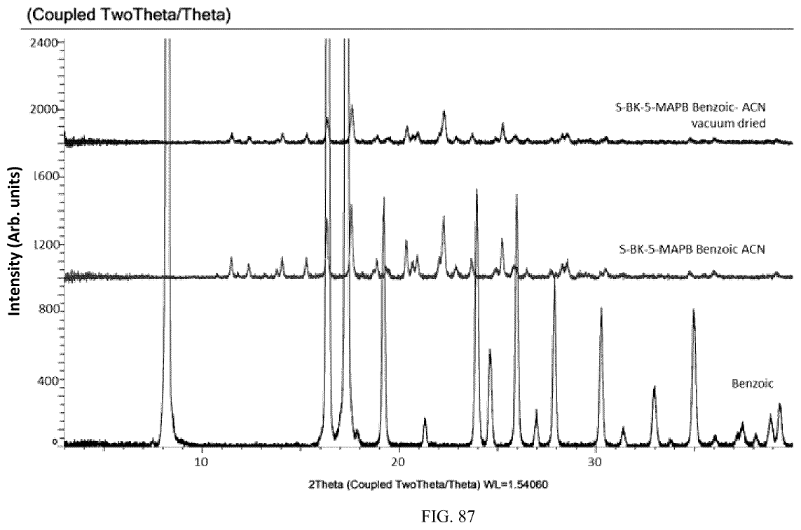

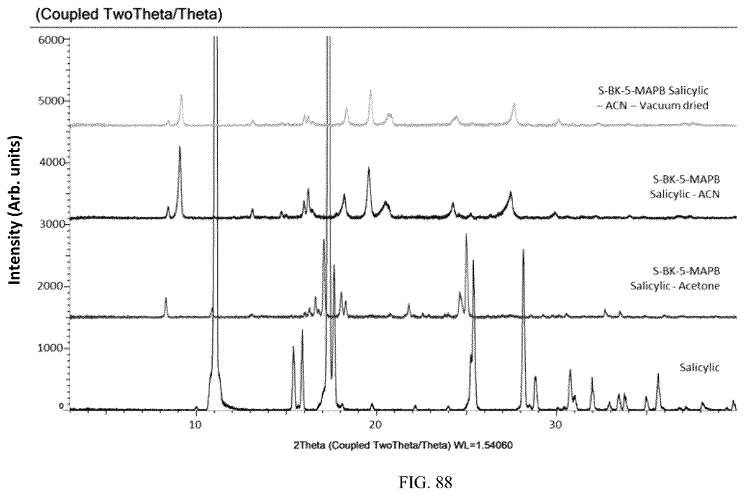

Salt studies of S-BK-5-MAPB were conducted using the conditions in Table 31. The below technique was used to generate the XRPD patterns in FIG. 63, FIG. 66, FIG. 68, FIG. 70, FIG. 72, FIG. 73, FIG. 74, FIG. 75, FIG. 76, FIG. 78, and FIG. 79 (For 40 mg of S-BK-5-MAPB, 1 vol. solvent=40 μL).

TABLE 31
Salt screening experiments

| | | | | PXRD |
|---|---|---|---|---|
| | | | | Result |
| Counter | | | PXRD | (Vacuum |
| ion | Solvent | Procedure/Comments | Result | Dried) |
| HCl | Acetone | 40 mg S-BK-5-MAPB (as freebase gel) + 10 | S-BK-5- | S-BK-5- |
| | | vols. solvent + 1.1:1 molar ratio counterion, | MAPB | MAPB |
| | | stirred at RT, light brown slurry after ~4 hours | Pattern 1B | Pattern |
| | | stirring, light brown slurry after overnight | | 1B |
| | | stirring, centrifuged to recover solids, | | |
| | | analyzed by PXRD, placed in vacuum oven at | | |
| | | 40° C., analyzed by PXRD, analyzed by | | |
| | | DSC/TGA | | |
| HBr | Acetone | 40 mg S-BK-5-MAPB (as freebase gel) + 10 | Low cryst. | N/A |
| | | vols. solvent + 1.1:1 molar ratio counterion, | pattern | |
| | | stirred at RT, very dark brown clear solution | | |
| | | after ~4 hours stirring, opened for | | |
| | | evaporation, brown/wet/gel-like solids after | | |
| | | overnight evaporation, brown/wet/gel-like | | |
| | | solids after 2 days evaporating, analyzed by | | |
| | | PXRD | | |
| H₂SO₄ | Acetone | 40 mg S-BK-5-MAPB (as freebase gel) + 10 | Low cryst. | N/A |
| | | vols. solvent + 1.1:1 molar ratio counterion, | pattern | |
| | | stirred at RT, very dark brown clear solution | | |
| | | after ~4 hours stirring, opened for | | |
| | | evaporation, brown/wet/gel-like solids after | | |
| | | overnight evaporation, brown/wet/gel-like | | |
| | | solids after 2 days evaporating, analyzed by | | |
| | | PXRD | | |
| H₃PO₄ | Acetone | 40 mg S-BK-5-MAPB (as freebase gel) + 10 | Low cryst. | N/A |
| | | vols. solvent + 1.1:1 molar ratio counterion, | pattern | |
| | | stirred at RT, very dark brown clear solution | | |
| | | after ~4 hours stirring, opened for | | |
| | | evaporation, brown/wet/gel-like solids after | | |
| | | overnight evaporation, brown/wet/gel-like | | |
| | | solids after 2 days evaporating, analyzed by | | |
| | | PXRD | | |
| HNO₃ | Acetone | 40 mg S-BK-5-MAPB (as freebase gel) + 10 | Low cryst. | N/A |
| | | vols. solvent + 1.1:1 molar ratio counterion, | pattern | |
| | | stirred at RT, red clear solution after ~4 hours | | |
| | | stirring, opened for evaporation, | | |
| | | orange/wet/gel-like solids after overnight | | |
| | | evaporation, orange/wet/gel-like solids after 2 | | |
| | | days evaporating, analyzed by PXRD | | |
| Methane | Acetone | 40 mg S-BK-5-MAPB (as freebase gel) + 10 | Low cryst. | N/A |
| sulfonic | | vols. solvent + 1.1:1 molar ratio counterion, | pattern | |
| | | stirred at RT, very dark brown clear solution | | |
| | | after ~4 hours stirring, opened for | | |
| | | evaporation, brown/wet/gel-like solids after | | |
| | | overnight evaporation, brown/wet/gel-like | | |
| | | solids after 2 days evaporating, analyzed by | | |
| | | PXRD | | |
| Tartaric | Acetone | 40 mg S-BK-5-MAPB (as freebase gel) + 10 | N/A | N/A |
| | | vols. solvent + 1.1:1 molar ratio counterion | | |
| | | (stock soln. in water), stirred at RT, orange | | |
| | | clear solution after ~4 hours stirring, opened | | |
| | | for evaporation, orange/cloudy solution with | | |
| | | gel-like solids after 3 days evaporation, | | |
| | | changed hot plate temperature to 40° C. for | | |
| | | evaporation, brown gel after 4 days | | |
| | | evaporating at 40° C., added 5 vols. 1,4- | | |
| | | Dioxane to reslurry, stirred at 40° C., brown | | |
| | | gel not suspended in solvent after ~4 hours | | |
| | | stirring, opened for evaporation, brown gel | | |
| | | after overnight evaporation, terminated | | |
| Succinic | Acetone | 40 mg S-BK-5-MAPB (as freebase gel) + 10 | N/A | N/A |
| | | vols. solvent + 1.1:1 molar ratio counterion | | |
| | | (stock soln. in water), stirred at RT, orange | | |
| | | cloudy solution/gel after ~4 hours stirring, | | |
| | | opened for evaporation, orange/cloudy | | |
| | | solution with gel-like solids after 3 days | | |
| | | evaporation, changed hot plate temperature to | | |
| | | 40° C. for evaporation, brown gel after 4 days | | |
| | | evaporating at 40° C., added 5 vols. 1,4- | | |
| | | Dioxane to reslurry, stirred at 40° C., brown | | |
| | | gel not suspended in solvent after ~4 hours | | |
| | | stirring, opened for evaporation, brown gel | | |
| | | after overnight evaporation, terminated | | |
| Oxalic | Acetone | 40 mg S-BK-5-MAPB (as freebase gel) + 10 | S-BK-5- | Lower |
| | | vols. solvent + 1.1:1 molar ratio counterion | MAPB | cryst. S- |
| | | (stock soln. in water), stirred at RT, orange | Pattern 9A | BK-5- |
| | | clear solution after ~4 hours stirring, opened | | MAPB |
| | | for evaporation, orange/cloudy solution with | | Pattern |
| | | gel-like solids after 3 days evaporation, | | 9A |
| | | changed hot plate temperature to 40° C. for | | |
| | | evaporation, orange/pasty solids after 4 days | | |
| | | evaporating, analyzed by PXRD, placed in | | |
| | | vacuum oven at 40° C., orange/pasty solids | | |
| | | analyzed by PXRD after overnight drying | | |
| Maleic | Acetone | 40 mg S-BK-5-MAPB (as freebase gel) + 10 | S-BK-5- | N/A |
| | | vols. solvent + 1.1:1 molar ratio counterion | MAPB | |
| | | (stock soln. in water), stirred at RT, orange | Pattern 10A | |
| | | clear solution after ~4 hours stirring, opened | | |
| | | for evaporation, orange/cloudy solution with | | |
| | | gel-like solids after 3 days evaporation, | | |
| | | changed hot plate temperature to 40° C. for | | |
| | | evaporation, brown gel after 4 days | | |
| | | evaporating at 40° C., added 5 vols. 1,4- | | |
| | | Dioxane to reslurry, stirred at 40° C., orange | | |
| | | slurry after overnight stirring, centrifuged to | | |
| | | recover wet orange solids, analyzed by | | |
| | | PXRD, placed in vacuum oven at 40° C. | | |
| Malic | Acetone | 40 mg S-BK-5-MAPB (as freebase gel) + 10 | N/A | N/A |
| | | vols. solvent + 1.1:1 molar ratio counterion | | |
| | | (stock soln. in water), stirred at RT, orange | | |
| | | clear solution after ~4 hours stirring, opened | | |
| | | for evaporation, orange/cloudy solution with | | |
| | | gel-like solids after 3 days evaporation, | | |
| | | changed hot plate temperature to 40° C. for | | |
| | | evaporation, brown gel after 4 days | | |
| | | evaporating at 40° C., added 5 vols. 1,4- | | |
| | | Dioxane to reslurry, stirred at 40° C., brown | | |
| | | gel not suspended in solvent after ~4 hours | | |
| | | stirring, opened for evaporation, brown gel | | |
| | | after overnight evaporation, terminated | | |
| Citric | Acetone | 40 mg S-BK-5-MAPB (as freebase gel) + 10 | N/A | N/A |
| | | vols. solvent + 1.1:1 molar ratio counterion | | |
| | | (stock soln. in water), stirred at RT, orange | | |
| | | cloudy solution/gel after ~4 hours stirring, | | |
| | | opened for evaporation, orange/cloudy | | |
| | | solution with gel-like solids after 3 days | | |
| | | evaporation, changed hot plate temperature to | | |
| | | 40° C. for evaporation, brown gel after 4 days | | |
| | | evaporating at 40° C., added 5 vols. 1,4- | | |
| | | Dioxane to reslurry, stirred at 40° C., brown | | |
| | | gel not suspended in solvent after ~4 hours | | |
| | | stirring, opened for evaporation, brown gel | | |
| | | after overnight evaporation, terminated | | |
| Fumaric | Acetone | 40 mg S-BK-5-MAPB (as freebase gel) + 10 | S-BK-5- | S-BK-5- |
| | | vols. solvent + 1.1:1 molar ratio counterion | MAPB | MAPB |
| | | (stock soln. in MeOH), stirred at RT, red clear | Pattern 13A | Pattern |
| | | solution after ~4 hours stirring, opened for | | 13A |
| | | evaporation, red gel after overnight | | |
| | | evaporation, added 5 vols. 1,4-Dioxane to | | |
| | | reslurry, stirred at RT, orange slurry after ~2 | | |
| | | hours stirring, orange slurry after overnight | | |
| | | stirring, centrifuged to recover solids, | | |
| | | analyzed by PXRD, placed in vacuum oven at | | |
| | | 40° C., analyzed by PXRD | | |
| Benzoic | Acetone | 40 mg S-BK-5-MAPB (as freebase gel) + 10 | N/A | N/A |
| | | vols. solvent + 1.1:1 molar ratio counterion | | |
| | | (stock soln. in MeOH), stirred at RT, red clear | | |
| | | solution after ~4 hours stirring, opened for | | |
| | | evaporation, brown gel after overnight | | |
| | | evaporation, added 5 vols. 1,4-Dioxane to | | |
| | | reslurry, stirred at RT, clear red solution after | | |
| | | ~2 hours stirring, opened for evaporation, red- | | |
| | | brown gel after overnight evaporation, added | | |
| | | 5 vols. chloroform to reslurry, stirred at RT, | | |
| | | clear brown solution after ~5 hours stirring, | | |
| | | opened for evaporation, red gel after | | |
| | | overnight evaporation, terminated | | |
| Salicylic | Acetone | 40 mg S-BK-5-MAPB (as freebase gel) + 10 | S-BK-5- | Similar to |
| | | vols. solvent + 1.1:1 molar ratio counterion | MAPB | S-BK-5- |
| | | (stock soln. in MeOH), stirred at RT, orange | Pattern 15A | MAPB |
| | | clear solution after ~4 hours stirring, opened | | Pattern |
| | | for evaporation, brown gel after overnight | | 15A |
| | | evaporation, added 5 vols. 1,4-Dioxane to | | |
| | | reslurry, stirred at RT, clear red solution after | | |
| | | ~2 hours stirring, opened for evaporation, | | |
| | | orange/wet/gel-like solids after overnight | | |
| | | evaporation, analyzed by PXRD, placed in | | |
| | | vacuum oven at 40° C., still sticky gel-like | | |
| | | solids after overnight drying, analyzed by | | |
| | | PXRD | | |

### EXAMPLE 37 Salt Studies of S-BK-5-MAPB Pure Enantiomer in MeOH; Water

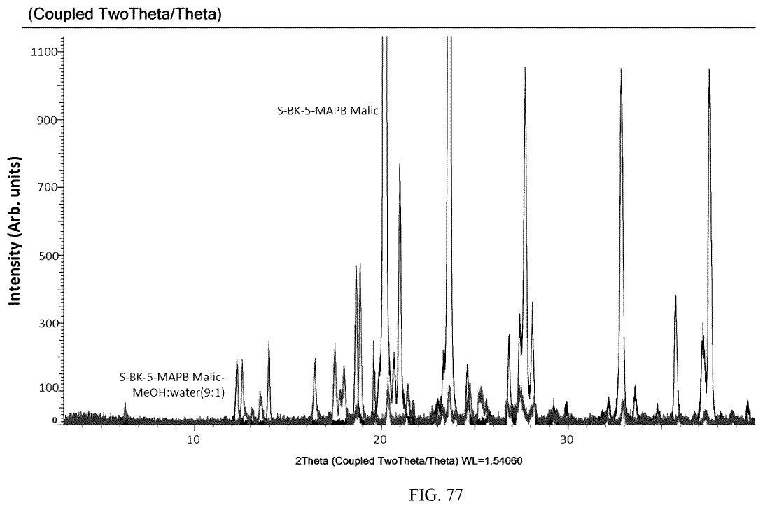

Salt studies of S-BK-5-MAPB were conducted using the conditions in Table 32. The below technique was used to generate the XRPD patterns in FIG. 67, FIG. 72, FIG. 73, FIG. 74, FIG. 75, FIG. 76, FIG. 77, FIG. 78, (For 40 mg of S-BK-5-MAPB, 1 vol. solvent=40 μL).

TABLE 32
Salt screening experiments

| | | | | PXRD |
|---|---|---|---|---|
| | | | | Result |
| Counter | | | PXRD | (Vacuum |
| ion | Solvent | Procedure/Comments | Result | Dried) |
| HCl | MeOH: | 40 mg S-BK-5-MAPB (as freebase gel) + 10 vols. | Similar to | N/A |
| | Water | solvent + 1.1:1 molar ratio counterion, stirred at | S-BK-5- | |
| | 9:1 | RT, orange clear solution after ~4 hours stirring, | MAPB | |
| | | opened for evaporation, orange/wet/gel-like | Pattern | |
| | | solids after overnight evaporation, | 1B | |
| | | orange/wet/gel-like solids after 2 days | | |
| | | evaporating, analyzed by PXRD, orange/wet/gel- | | |
| | | like solids after 2 days evaporating, analyzed by | | |
| | | PXRD | | |
| HBr | MeOH: | 40 mg S-BK-5-MAPB (as freebase gel) + 10 vols. | Low | N/A |
| | Water | solvent + 1.1:1 molar ratio counterion, stirred at | cryst. | |
| | 9:1 | RT, orange clear solution after ~4 hours stirring, | pattern | |
| | | opened for evaporation, orange/wet/gel-like | | |
| | | solids after overnight evaporation, | | |
| | | orange/wet/gel-like solids after 2 days | | |
| | | evaporating, analyzed by PXRD | | |
| H₂SO₄ | MeOH: | 40 mg S-BK-5-MAPB (as freebase gel) + 10 vols. | Low | N/A |
| | Water | solvent + 1.1:1 molar ratio counterion, stirred at | cryst. | |
| | 9:1 | RT, orange clear solution after ~4 hours stirring, | pattern | |
| | | opened for evaporation, orange/wet/gel-like | | |
| | | solids after overnight evaporation, | | |
| | | orange/wet/gel-like solids after 2 days | | |
| | | evaporating, analyzed by PXRD | | |
| H₃PO₄ | MeOH: | 40 mg S-BK-5-MAPB (as freebase gel) + 10 vols. | Low | N/A |
| | Water | solvent + 1.1:1 molar ratio counterion, stirred at | cryst. | |
| | 9:1 | RT, red clear solution after ~4 hours stirring, | pattern | |
| | | opened for evaporation, orange/wet/gel-like | | |
| | | solids after overnight evaporation, | | |
| | | orange/wet/gel-like solids after 2 days | | |
| | | evaporating, analyzed by PXRD | | |
| HNO₃ | MeOH: | 40 mg S-BK-5-MAPB (as freebase gel) + 10 vols. | Low | N/A |
| | Water | solvent + 1.1:1 molar ratio counterion, stirred at | cryst. | |
| | 9:1 | RT, orange clear solution after ~4 hours stirring, | pattern | |
| | | opened for evaporation, orange/wet/gel-like | | |
| | | solids after overnight evaporation, | | |
| | | orange/wet/gel-like solids after 2 days | | |
| | | evaporating, analyzed by PXRD | | |
| Methane | MeOH: | 40 mg S-BK-5-MAPB (as freebase gel) + 10 vols. | Low | N/A |
| sulfonic | Water | solvent + 1.1:1 molar ratio counterion, stirred at | cryst. | |
| | 9:1 | RT, orange clear solution after ~4 hours stirring, | pattern | |
| | | opened for evaporation, orange/wet/gel-like | | |
| | | solids after overnight evaporation, | | |
| | | orange/wet/gel-like solids after 2 days | | |
| | | evaporating, analyzed by PXRD | | |
| Tartaric | MeOH: | 40 mg S-BK-5-MAPB (as freebase gel) + 10 vols. | N/A | N/A |
| | Water | solvent + 1.1:1 molar ratio counterion (stock soln. | | |
| | 9:1 | in water), stirred at RT, orange cloudy | | |
| | | solution/gel after ~4 hours stirring, opened for | | |
| | | evaporation, orange/cloudy solution with gel-like | | |
| | | solids after 3 days evaporation, changed hot plate | | |
| | | temperature to 40° C. for evaporation, brown gel | | |
| | | after 4 days evaporating at 40° C., added 5 vols. | | |
| | | chloroform to reslurry, stirred at 40° C., brown gel | | |
| | | not suspended in solvent after ~4 hours stirring, | | |
| | | opened for evaporation, brown gel after overnight | | |
| | | evaporation, terminated | | |
| Succinic | MeOH: | 40 mg S-BK-5-MAPB (as freebase gel) + 10 vols. | N/A | N/A |
| | Water | solvent + 1.1:1 molar ratio counterion (stock soln. | | |
| | 9:1 | in water), stirred at RT, orange cloudy | | |
| | | solution/gel after ~4 hours stirring, opened for | | |
| | | evaporation, orange/cloudy solution with gel-like | | |
| | | solids after 3 days evaporation, changed hot plate | | |
| | | temperature to 40° C. for evaporation, brown gel | | |
| | | after 4 days evaporating at 40° C., added 5 vols. | | |
| | | chloroform to reslurry, stirred at 40° C., brown gel | | |
| | | not suspended in solvent after ~4 hours stirring, | | |
| | | opened for evaporation, brown gel after overnight | | |
| | | evaporation, terminated | | |
| Oxalic | MeOH: | 40 mg S-BK-5-MAPB (as freebase gel) + 10 vols. | S-BK-5- | N/A |
| | Water | solvent + 1.1:1 molar ratio counterion (stock soln. | MAPB | |
| | 9:1 | in water), stirred at RT, orange cloudy | Pattern | |
| | | solution/gel after ~4 hours stirring, opened for | 9A | |
| | | evaporation, orange/cloudy solution with gel-like | | |
| | | solids after 3 days evaporation, changed hot plate | | |
| | | temperature to 40° C. for evaporation, | | |
| | | orange/pasty solids after 4 days evaporating, | | |
| | | analyzed by PXRD | | |
| Maleic | MeOH: | 40 mg S-BK-5-MAPB (as freebase gel) + 10 vols. | S-BK-5- | N/A |
| | Water | solvent + 1.1:1 molar ratio counterion (stock soln. | MAPB | |
| | 9:1 | in water), stirred at RT, orange cloudy | Pattern | |
| | | solution/gel after ~4 hours stirring, opened for | 10A with | |
| | | evaporation, orange/cloudy solution with gel-like | a new | |
| | | solids after 3 days evaporation, changed hot plate | peak at | |
| | | temperature to 40° C. for evaporation, brown gel | 16.4° | |
| | | after 4 days evaporating at 40° C., added 5 vols. | | |
| | | chloroform to reslurry, stirred at 40° C., brown gel | | |
| | | not suspended in solvent after ~4 hours stirring, | | |
| | | opened for evaporation, orange-brown/pasty | | |
| | | solids after overnight evaporation, analyzed by | | |
| | | PXRD | | |
| Malic | MeOH: | 40 mg S-BK-5-MAPB (as freebase gel) + 10 vols. | S-BK-5- | N/A |
| | Water | solvent + 1.1:1 molar ratio counterion (stock soln. | MAPB | |
| | 9:1 | in water), stirred at RT, orange cloudy | Pattern | |
| | | solution/gel after ~4 hours stirring, opened for | 11A | |
| | | evaporation, orange/cloudy solution with gel-like | | |
| | | solids after 3 days evaporation, changed hot plate | | |
| | | temperature to 40° C. for evaporation, brown gel | | |
| | | after 4 days evaporating at 40° C., added 5 vols. | | |
| | | chloroform to reslurry, stirred at 40° C., brown gel | | |
| | | not suspended in solvent after ~4 hours stirring, | | |
| | | opened for evaporation, orange-brown/pasty | | |
| | | solids after overnight evaporation, analyzed by | | |
| | | PXRD, placed in vacuum oven at 40° C. | | |
| Citric | MeOH: | 40 mg S-BK-5-MAPB (as freebase gel) + 10 vols. | Low | N/A |
| | Water | solvent + 1.1:1 molar ratio counterion (stock soln. | cryst. | |
| | 9:1 | in water), stirred at RT, orange cloudy | pattern | |
| | | solution/gel after ~4 hours stirring, opened for | | |
| | | evaporation, orange/cloudy solution with gel-like | | |
| | | solids after 3 days evaporation, centrifuged to | | |
| | | recover solids, red-brown/pasty solids analyzed | | |
| | | by PXRD | | |
| Fumaric | MeOH: | 40 mg S-BK-5-MAPB (as freebase gel) + 10 vols. | Low | N/A |
| | Water | solvent + 1.1:1 molar ratio counterion (stock soln. | cryst. | |
| | 9:1 | in MeOH), stirred at RT, orange clear solution | pattern | |
| | | after ~4 hours stirring, opened for evaporation, | | |
| | | red/clear solutions after overnight evaporation, | | |
| | | orange/wet/gel-like solids after 2 days | | |
| | | evaporating, analyzed by PXRD | | |
| Benzoic | MeOH: | 40 mg S-BK-5-MAPB (as freebase gel) + 10 vols. | N/A | N/A |
| | Water | solvent + 1.1:1 molar ratio counterion (stock soln. | | |
| | 9:1 | in MeOH), stirred at RT, orange clear solution | | |
| | | after ~4 hours stirring, opened for evaporation, | | |
| | | red/clear solution after overnight evaporation, | | |
| | | red-brown gel after 2 days evaporation, added 5 | | |
| | | vols. chloroform to reslurry, stirred at RT, cloudy | | |
| | | orange solution after ~5 hours stirring, opened for | | |
| | | evaporation, red gel after overnight evaporation, | | |
| | | terminated | | |
| Salicylic | MeOH: | 40 mg S-BK-5-MAPB (as freebase gel) + 10 vols. | N/A | N/A |
| | Water | solvent + 1.1:1 molar ratio counterion (stock soln. | | |
| | 9:1 | in MeOH), stirred at RT, orange clear solution | | |
| | | after ~4 hours stirring, opened for evaporation, | | |
| | | red/clear solution after overnight evaporation, | | |
| | | red-brown gel after 2 days evaporation, added 5 | | |
| | | vols. chloroform to reslurry, stirred at RT, cloudy | | |
| | | orange solution after ~5 hours stirring, opened for | | |
| | | evaporation, red gel after overnight evaporation, | | |
| | | terminated | | |

### EXAMPLE 38 Salt Studies of S-BK-5-MAPB Pure Enantiomer in ACN

Salt studies of S-BK-5-MAPB were conducted using the conditions in Table 33. The below technique was used to generate the XRPD patterns in FIG. 64, FIG. 69, and FIG. 71 (For 40 mg of S-BK-5-MAPB, 1 vol. solvent=40 μL).

TABLE 33
Salt screening experiments

| | | | | PXRD |
|---|---|---|---|---|
| | | | | Result |
| Counter | | | PXRD | (Vacuum |
| ion | Solvent | Procedure/Comments | Result | Dried) |
| HCl | ACN | 40 mg S-BK-5-MAPB (as freebase gel) + 5 vols. | Lower | N/A |
| | | solvent + 1.1:1 molar ratio counterion, stirred at | cryst. | |
| | | 40° C., brown/clear solution after ~45 minutes | Pattern | |
| | | stirring, opened for evaporation, brown gel after | 1B | |
| | | overnight evaporation, added 5 vols. 1,4-Dioxane | | |
| | | to reslurry, stirred at 40° C., cloudy/orange | | |
| | | solution with brown unsuspended gel after 4 days, | | |
| | | opened for evaporation, brown pasty solids after | | |
| | | overnight evaporation, analyzed by PXRD | | |
| HBr | ACN | 40 mg S-BK-5-MAPB (as freebase gel) + 5 vols. | N/A | N/A |
| | | solvent + 1.1:1 molar ratio counterion, stirred at | | |
| | | 40° C., brown/clear solution after ~45 minutes | | |
| | | stirring, opened for evaporation, brown gel after | | |
| | | overnight evaporation, added 5 vols. 1,4-Dioxane | | |
| | | to reslurry, stirred at 40° C., oiled out after 4 days, | | |
| | | opened for evaporation, brown gel after overnight | | |
| | | evaporation, added 5 vols. chloroform to reslurry, | | |
| | | stirred at 40° C., brown/cloudy solution after ~1.5 | | |
| | | hours stirring, opened for evaporation, brown gel | | |
| | | after 6 days evaporating, terminated | | |
| H₂SO₄ | ACN | 40 mg S-BK-5-MAPB (as freebase gel) + 5 vols. | N/A | N/A |
| | | solvent + 1.1:1 molar ratio counterion, stirred at | | |
| | | 40° C., brown/clear solution after ~45 minutes | | |
| | | stirring, opened for evaporation, brown gel after | | |
| | | overnight evaporation, added 5 vols. 1,4-Dioxane | | |
| | | to reslurry, stirred at 40° C., oiled out after 4 days, | | |
| | | opened for evaporation, brown gel after overnight | | |
| | | evaporation, added 5 vols. chloroform to reslurry, | | |
| | | stirred at 40° C., cloudy solution with | | |
| | | unsuspended gel after ~1.5 hours stirring, opened | | |
| | | for evaporation, brown gel after overnight | | |
| | | evaporation, terminated | | |
| H₂SO₄ | ACN | 40 mg S-BK-5-MAPB (as freebase gel) + 5 vols. | S-BK-5- | Pattern |
| | | solvent + 1.1:1 molar ratio counterion, stirred at | MAPB | 3A |
| | | 40° C., brown/thick slurry after ~1 hour stirring, 5 | Pattern | |
| | | additional vols. solvent added, brown slurry after | 3A | |
| | | overnight stirring, centrifuged to recover solids, | | |
| | | analyzed by PXRD, placed in vacuum oven at 40° | | |
| | | C., analyzed by PXRD after overnight drying, | | |
| | | analyzed by DSC/TGA/NMR | | |
| H₃PO | ACN | 40 mg S-BK-5-MAPB (as freebase gel) + 5 vols. | N/A | N/A |
| | | solvent + 1.1:1 molar ratio counterion, stirred at | | |
| | | 40° C., brown/cloudy solution with very little | | |
| | | unsuspended gel after ~45 minutes stirring, | | |
| | | opened for evaporation, brown gel after overnight | | |
| | | evaporation, added 5 vols. 1,4-Dioxane to | | |
| | | reslurry, stirred at 40° C., cloudy/orange solution | | |
| | | with brown unsuspended gel after 4 days, opened | | |
| | | for evaporation, brown gel after overnight | | |
| | | evaporation, added 5 vols. DCM to reslurry, | | |
| | | stirred at 40° C., cloudy solution with | | |
| | | unsuspended gel after ~1.5 hours stirring, opened | | |
| | | for evaporation, orange gel after overnight | | |
| | | evaporation, terminated | | |
| H₃PO₄ | ACN | 40 mg S-BK-5-MAPB (as freebase gel) + 5 vols. | Low | N/A |
| | | solvent + 1.1:1 molar ratio counterion, stirred at | cryst. | |
| | | 40° C., brown/cloudy solution with very little | Pattern | |
| | | unsuspended gel after ~45 minutes stirring, | | |
| | | opened for evaporation, brown gel after overnight | | |
| | | evaporation, added 5 vols. chloroform to reslurry, | | |
| | | stirred at 40° C., cloudy/orange solution with | | |
| | | brown unsuspended gel after 4 days, opened for | | |
| | | evaporation, brown gel after overnight | | |
| | | evaporation, added 5 vols. EtOAc to reslurry, | | |
| | | stirred at 40° C., cloudy solution with | | |
| | | unsuspended gel after ~1.5 hours stirring, opened | | |
| | | for evaporation, orange solids on walls of vial | | |
| | | after overnight evaporation, analyzed by PXRD | | |
| HNO₃ | ACN | 40 mg S-BK-5-MAPB (as freebase gel) + 5 vols. | Low | N/A |
| | | solvent + 1.1:1 molar ratio counterion, stirred at | cryst. | |
| | | 40° C., brown/clear solution after ~45 minutes | pattern | |
| | | stirring, opened for evaporation, sticky orange | | |
| | | solids after overnight evaporation, analyzed by | | |
| | | PXRD | | |
| Methane | ACN | 40 mg S-BK-5-MAPB (as freebase gel) + 5 vols. | N/A | N/A |
| sulfonic | | solvent + 1.1:1 molar ratio counterion, stirred at | | |
| | | 40° C., brown/clear solution after ~45 minutes | | |
| | | stirring, opened for evaporation, brown gel after | | |
| | | overnight evaporation, added 5 vols. 1,4-Dioxane | | |
| | | to reslurry, stirred at 40° C., clear orange solution | | |
| | | after 4 days, opened for evaporation, brown gel | | |
| | | after overnight evaporation, added 5 vols. | | |
| | | chloroform to reslurry, stirred at 40° C., clear | | |
| | | orange solution with unsuspended gel after ~1.5 | | |
| | | hours stirring, opened for evaporation, brown gel | | |
| | | after 6 days evaporating, terminated | | |
| Tartaric | ACN | 40 mg S-BK-5-MAPB (as freebase gel) + 5 vols. | Tartaric | N/A |
| | | solvent + 1.1:1 molar ratio counterion (stock | acid | |
| | | solution in water), stirred at 40° C., brown/cloudy | peaks | |
| | | solution with very little unsuspended gel after ~45 | | |
| | | minutes stirring, opened for evaporation, | | |
| | | cloudy/orange solution with unsuspended brown | | |
| | | gel after overnight evaporation, brown gel after 4 | | |
| | | days, added 5 vols. DCM to reslurry, stirred at 40° | | |
| | | C., brown cloudy solution with unsuspended | | |
| | | brown gel, opened for evaporation, brown pasty | | |
| | | solids after overnight evaporation, analyzed by | | |
| | | PXRD | | |
| Succinic | ACN | 40 mg S-BK-5-MAPB (as freebase gel) + 5 vols. | N/A | N/A |
| | | solvent + 1.1:1 molar ratio counterion (stock | | |
| | | solution in water), stirred at 40° C., brown/clear | | |
| | | solution after ~45 minutes stirring, opened for | | |
| | | evaporation, cloudy/orange solution with | | |
| | | unsuspended brown gel after overnight | | |
| | | evaporation, brown gel after 4 days, added 5 vols. | | |
| | | DCM to reslurry, stirred at 40° C., brown cloudy | | |
| | | solution after ~5 hours stirring, opened for | | |
| | | evaporation, brown gel after overnight | | |
| | | evaporation, added 5 vols. EtOAc to reslurry, | | |
| | | stirred at 40° C., cloudy solution with | | |
| | | unsuspended gel after ~1.5 hours stirring, opened | | |
| | | for evaporation, brown gel after overnight | | |
| | | evaporation, terminated | | |
| Oxalic | ACN | 40 mg S-BK-5-MAPB (as freebase gel) + 5 vols. | S-BK-5- | N/A |
| | | solvent + 1.1:1 molar ratio counterion (stock | MAPB | |
| | | solution in water), stirred at 40° C., orange/clear | Pattern | |
| | | solution after ~1 hour stirring, opened for | 9A with | |
| | | evaporation, cloudy/orange solution with | new | |
| | | unsuspended brown gel after overnight | peaks at | |
| | | evaporation, sticky orange/brown solids after 4 | 13.2°. | |
| | | days, analyzed by PXRD | 16.5°, | |
| | | | 18.9°, | |
| | | | and 27.9° | |
| Maleic | ACN | 40 mg S-BK-5-MAPB (as freebase gel) + 5 vols. | Lower | N/A |
| | | solvent + 1.1:1 molar ratio counterion (stock | cryst. S- | |
| | | solution in water), stirred at 40° C., orange/clear | BK-5- | |
| | | solution after ~1 hour stirring, opened for | MAPB | |
| | | evaporation, cloudy/orange solution with | Pattern | |
| | | unsuspended brown gel after overnight | 10A with | |
| | | evaporation, brown gel after 4 days, added 5 vols. | a peak at | |
| | | DCM to reslurry, stirred at 40° C., orange slurry | 16.4° | |
| | | after overnight stirring, centrifuged to recover | (similar | |
| | | solids, analyzed by PXRD | to | |
| | | | NB1018 | |
| | | | A-75-25) | |
| Malic | ACN | 40 mg S-BK-5-MAPB (as freebase gel) + 5 vols. | N/A | N/A |
| | | solvent + 1.1:1 molar ratio counterion (stock | | |
| | | solution in water), stirred at 40° C., brown/cloudy | | |
| | | solution with very little unsuspended gel after ~45 | | |
| | | minutes stirring, opened for evaporation, | | |
| | | cloudy/orange solution with unsuspended brown | | |
| | | gel after overnight evaporation, brown gel after 4 | | |
| | | days, added 5 vols. DCM to reslurry, stirred at 40° | | |
| | | C., brown cloudy solution with unsuspended | | |
| | | brown gel, opened for evaporation, brown gel | | |
| | | after overnight evaporation, added 5 vols. EtOAc | | |
| | | to reslurry, stirred at 40° C., cloudy solution with | | |
| | | unsuspended gel after ~1.5 hours stirring, opened | | |
| | | for evaporation, brown gel after overnight | | |
| | | evaporation, terminated | | |
| Citric | ACN | 40 mg S-BK-5-MAPB (as freebase gel) + 5 vols. | Low | N/A |
| | | solvent + 1.1:1 molar ratio counterion (stock | cryst. | |
| | | solution in water), stirred at 40° C., brown/cloudy | Pattern | |
| | | solution with very little unsuspended gel after ~45 | | |
| | | minutes stirring, opened for evaporation, | | |
| | | cloudy/orange solution with unsuspended brown | | |
| | | gel after overnight evaporation, brown gel after 4 | | |
| | | days, added 5 vols. chloroform to reslurry, stirred | | |
| | | at 40° C., brown cloudy solution with | | |
| | | unsuspended brown gel, opened for evaporation, | | |
| | | brown pasty solids after overnight evaporation, | | |
| | | analyzed by PXRD | | |
| Fumaric | ACN | 34 mg S-BK-5-MAPB (as freebase gel) + 5 vols. | Low | N/A |
| | | solvent + 1.1:1 molar ratio counterion (stock | cryst. | |
| | | solution in MeOH), stirred at 40° C., orange/clear | Pattern | |
| | | solution after ~1 hour stirring, opened for | 13B | |
| | | evaporation, sticky orange solids after overnight | | |
| | | evaporation, analyzed by PXRD | | |
| Benzoic | ACN | 40 mg S-BK-5-MAPB (as freebase gel) + 5 vols. | S-BK-5- | Pattern |
| | | solvent + 1.1:1 molar ratio counterion (stock | MAPB | 14A |
| | | solution in MeOH), stirred at 40° C., orange/clear | Pattern | |
| | | solution after ~1 hour stirring, opened for | 14A | |
| | | evaporation, brown gel after overnight | | |
| | | evaporation, added 5 vols. DCM to reslurry, | | |
| | | stirred at 40° C., clear orange solution after 4 | | |
| | | days, opened for evaporation, brown gel after | | |
| | | overnight evaporation, added 5 vols. EtOAc to | | |
| | | reslurry, stirred at 40° C., orange/thin slurry after | | |
| | | overnight stirring, centrifuged to recover solids, | | |
| | | analyzed by PXRD, placed in vacuum oven | | |
| | | at 40° C., analyzed by PXRD after overnight | | |
| | | drying, analyzed by DSC/NMR | | |
| Salicylic | ACN | 40 mg S-BK-5-MAPB (as freebase gel) + 5 vols. | S-BK-5- | Lower |
| | | solvent + 1.1:1 molar ratio counterion (stock | MAPB | cryst. S- |
| | | solution in MeOH), stirred at 40° C., brown/clear | Pattern | BK-5- |
| | | solution after ~45 minutes stirring, opened for | 15B | MAPB |
| | | evaporation, brown gel 1 after overnight | | Pattern |
| | | evaporation, added 5 vols. DCM to reslurry, | | 15B |
| | | stirred at 40° C., clear orange solution after 4 | | |
| | | days, opened for evaporation, brown pasty | | |
| | | solids after overnight evaporation, analyzed by | | |
| | | PXRD, placed in vacuum oven at 40° C., | | |
| | | analyzed by PXRD after overnight drying, | | |
| | | analyzed by DSC/NMR | | |

### EXAMPLE 39 Salt Studies of S-BK-5-MAPB Pure Enantiomer in THF or Toluene

Salt studies of S-BK-5-MAPB were conducted using the conditions in Table 34.

TABLE 34
Salt screening experiments

| | | | | PXRD |
|---|---|---|---|---|
| | | | | Result |
| Counter | | | PXRD | (Vacuum |
| ion | Solvent | Procedure/Comments | Result | Dried) |
| HCl | THF | 40 mg S-BK-5-MAPB (as freebase gel) + 5 vols. | S-BK-5- | N/A |
| | | solvent + 1.1:1 molar ratio counterion, stirred at | MAPB | |
| | | 40° C., oiled out after overnight stirring, opened | Pattern | |
| | | for evaporation, sticky orange/brown solids after | 1B | |
| | | 4 days, analyzed by PXRD | | |
| HCl | Toluene | 40 mg S-BK-5-MAPB (as freebase gel) + 5 vols. | S-BK-5- | N/A |
| | | solvent + 1.1:1 molar ratio counterion, stirred at | MAPB | |
| | | 40° C., yellow/cloudy solution with unsuspended | Pattern | |
| | | orange gel after ~1 hour stirring, yellow/cloudy | 1B | |
| | | solution with unsuspended brown gel after | | |
| | | overnight stirring, opened for evaporation, sticky | | |
| | | orange/brown solids after 4 days, analyzed by | | |
| | | PXRD | | |

### EXAMPLE 40: S-6-MBPB Freebase Conversion/Liquid-Liquid Extraction

Liquid-Liquid Extraction (LLE) was used to isolate S-6-MBPB Freebase from Pattern 1A (S-6-MBPB HCl, Pure Enantiomer, FIG. 98) using the conditions shown in Table 35 (for 50 mg of S-6-MBPB HCl Pure Enantiomer, 1 vol. solvent is equivalent to 50 μL). Table 37 describes liquid-liquid extraction conditions of S-6-MBPB.

TABLE 35
S-6-MBPB Freebase conversion

| Base | Procedure/Comments |
|---|---|
| NaOH | 50 mg API (as HCL salt) + base stock soln. in |
| | water (1.1:1 molar ratio), stirred at RT, brown |
| | gel-like solids unsuspended in water, pipetted |
| | out solvent, left vial open at 40° C. to dry, wet |
| | brown gel after overnight drying |

TABLE 36
Liquid-liquid extraction of S-6-MBPB

| Base | Solvent (Density) | Procedure/Comments |
|---|---|---|
| NaOH | EtOAc (0.902 g/mL) | 50 mg API (as HCl salt) + 18.4 vols. |
| | | solvent + base stock soln. in water |
| | | (1.1:1 molar ratio), vigorously |
| | | shaken and allowed to separate, |
| | | pipetted out top organic layer, |
| | | allowed organic layer to evaporate |
| | | at 40° C., resulting brown gel |
| | | weighed ~18.5 mg after overnight |
| | | evaporation, analyzed by NMR |

### EXAMPLE 41. Scale Up of S-6-MBPB Liquid-Liquid Extraction

Scale ups of the S-6-MBPB liquid-liquid extraction expt. were performed to isolate large amounts of the Freebase API from the S-6-MBPB HCl salt. ˜1 g API (as HCl salt) plus 18.4 vols. (18.4 mL) of EtOAc were added to Na 1H soln. in water (10 mg/mL or 5 mg/mL) for 1.1:1 base:salt molar ratio. Organic phase was then recovered, the solvent was allowed to evaporate at RT, and the resulting brown gel was analyzed by NMR. Table 37 describes scale up conditions of liquid-liquid extraction conditions of S-6-MBPB.

TABLE 37
Liquid-liquid extraction scale up experiments of S-6-MBPB

| | Solvent | |
|---|---|---|
| Base | (Density) | Procedure/Comments |
| NaOH | EtOAc | 1 g API (as HCl salt) + 18.4 vols. solvent + base stock soln. in water (1.1:1 |
| | (0.902 g/mL) | molar ratio), vigorously shaken and allowed to separate |
| NaOH | EtOAc | 1 g API (as HCl salt) + 18.4 vols. solvent + base stock soln. in water (1.1:1 |
| | (0.902 g/mL) | molar ratio), vigorously shaken and allowed to separate |
| NaOH | EtOAc | The top organic layers of S-6-MBPB were pipetted out and combined, |
| | (0.902 g/mL) | concentration estimated as ~46.6 mg/mL, analyzed by NMR |
| NaOH | EtOAc | 1 g API (as HCl salt) + 18.4 vols. solvent + base stock soln. in water (1.1:1 |
| | (0.902 g/mL) | molar ratio), vigorously shaken and allowed to separate |
| NaOH | EtOAc | The top organic layers of S-6-MBPB were pipetted out and combined, |
| | (0.902 g/mL) | concentration estimated as ~46.6 mg/mL, concentration closer to ~47.5 |
| | | mg/mL |
| NaOH | EtOAc | 1 g API (as HCl salt) + 18.4 vols. solvent + base stock soln. in water (1.1:1 |
| | (0.902 g/mL) | molar ratio), vigorously shaken and allowed to separate |
| NaOH | EtOAc | The top organic layers of S-6-MBPB were pipetted out and combined, |
| | (0.902 g/mL) | concentration estimated as ~46.6 mg/mL, concentration closer to ~47.5 |
| | | mg/mL |
| NaOH | EtOAc | 1 g API (as HCl salt) + 18.4 vols. solvent + base stock soln. in water (1.1:1 |
| | (0.902 g/mL) | molar ratio), vigorously shaken and allowed to separate |
| NaOH | EtOAc | The top organic layers of S-6-MBPB were pipetted out and combined, |
| | (0.902 g/mL) | concentration estimated as ~47.5 mg/mL, concentration closer to ~45.9 |
| | | mg/mL |

### EXAMPLE 42 Salt Study Experiments of S-6-MBPB Pure Enantiomer in Acetone

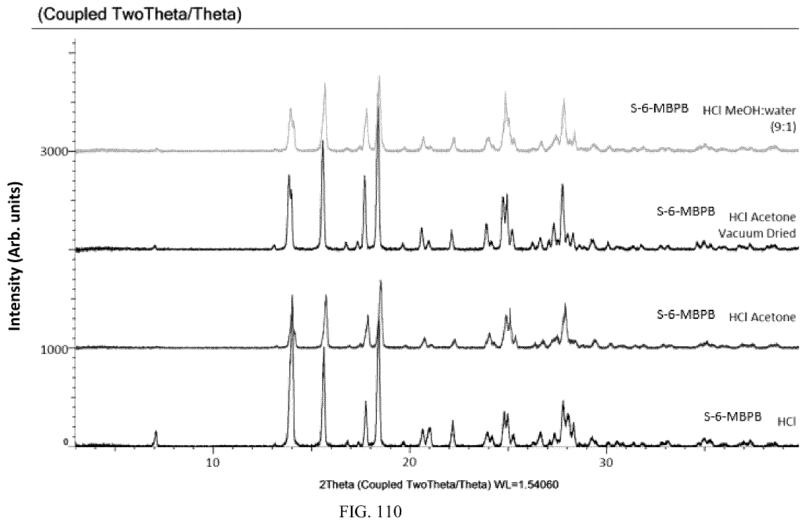

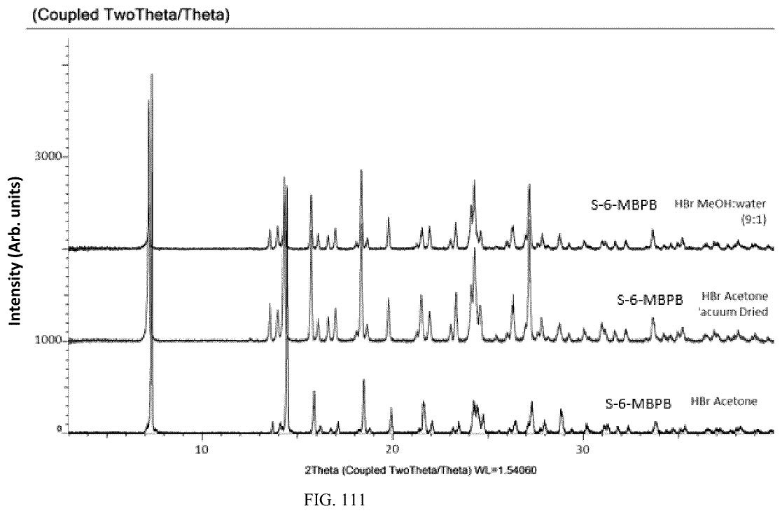

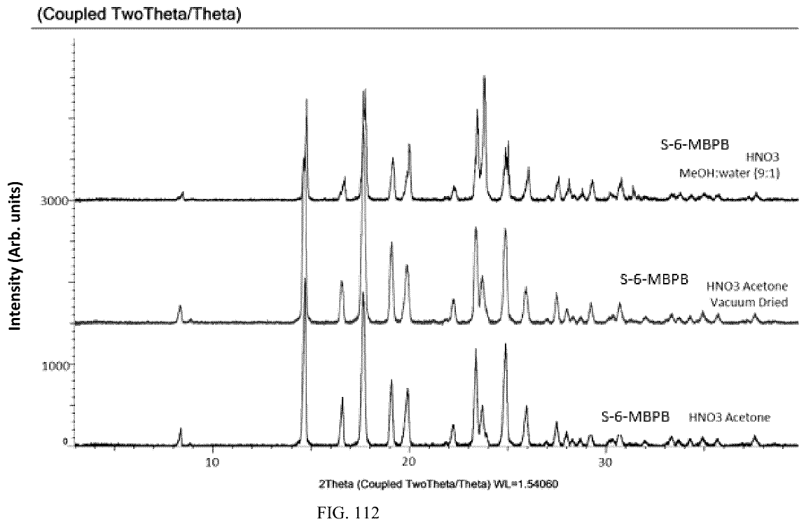

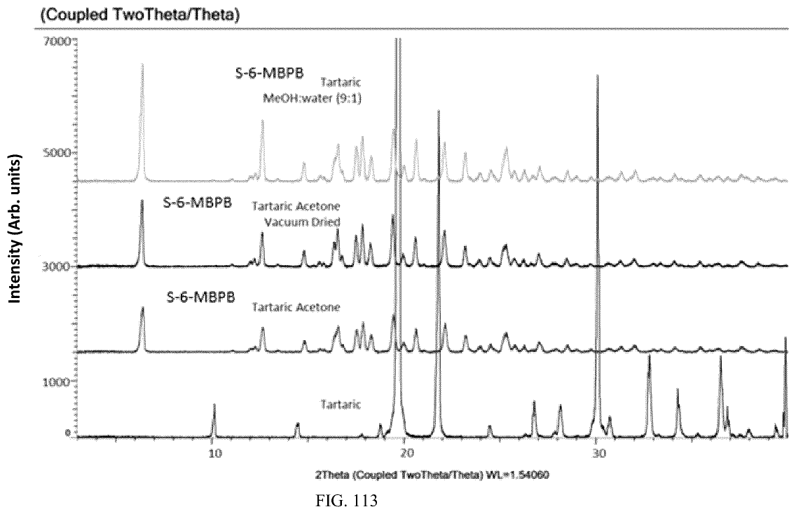

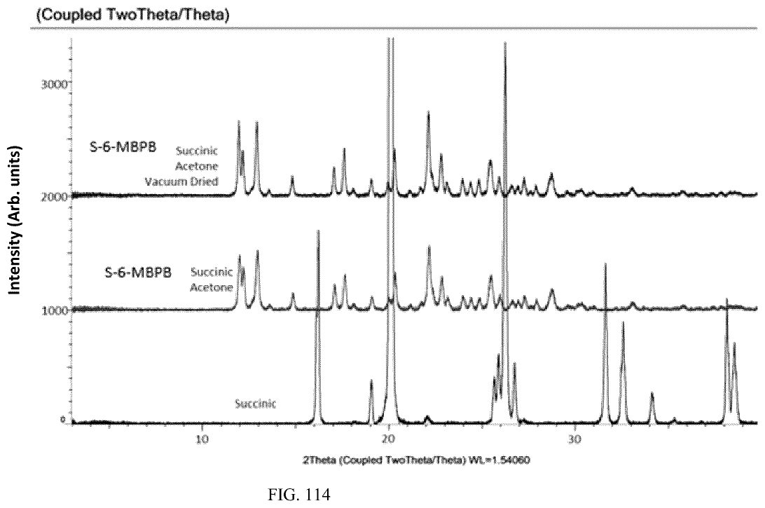

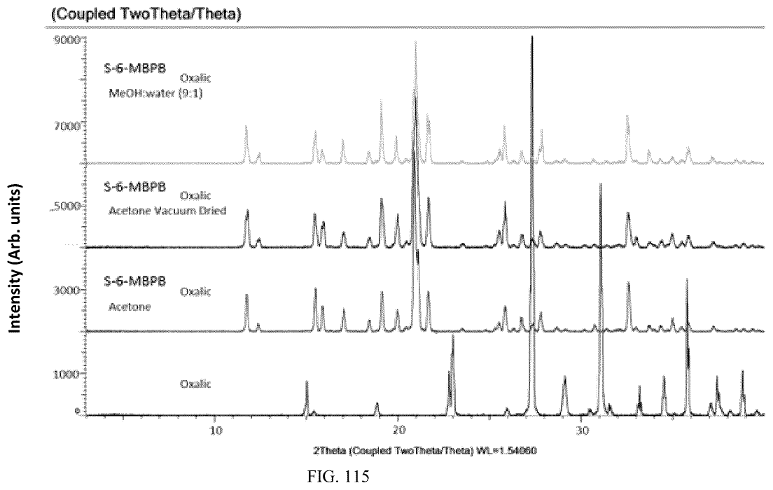

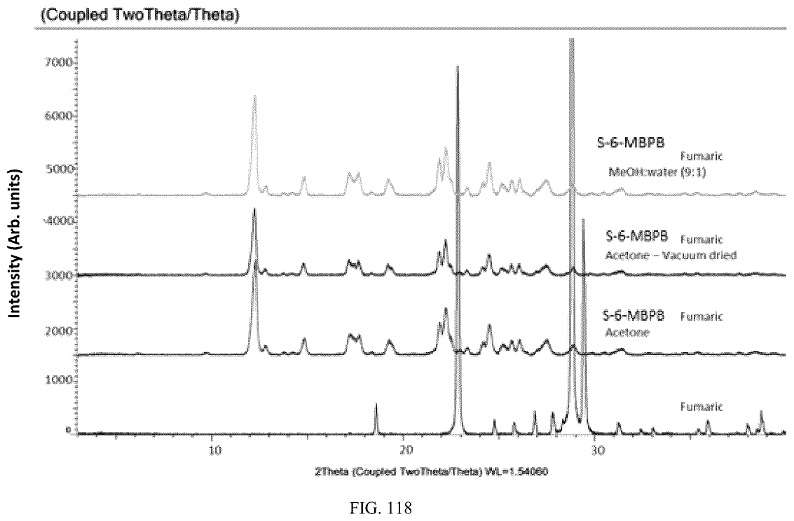

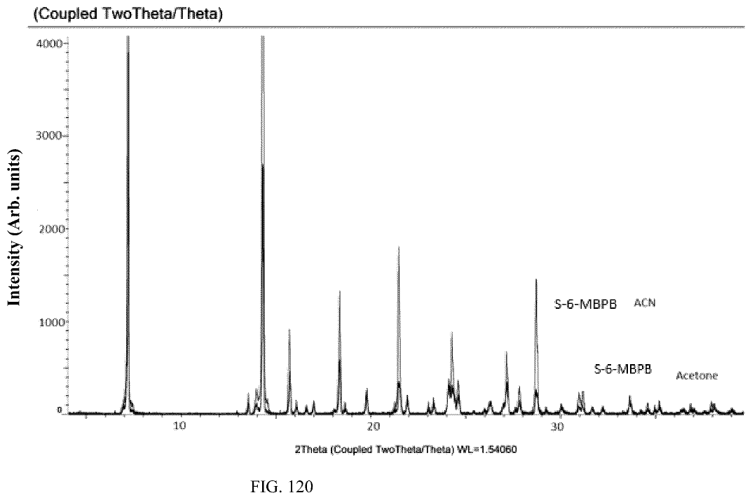

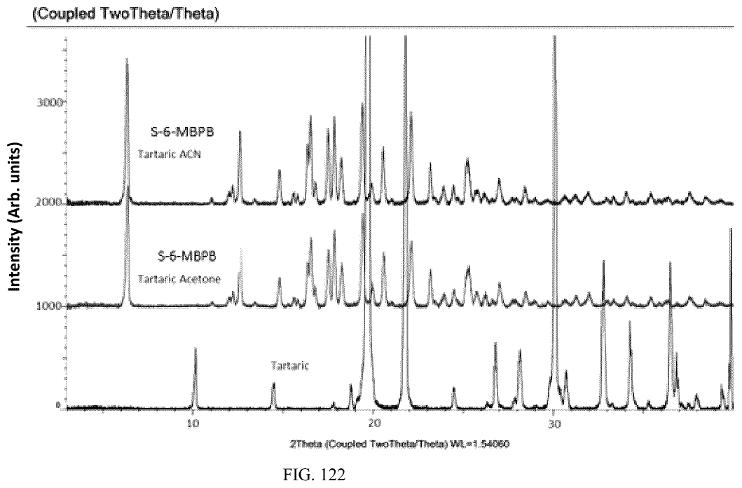

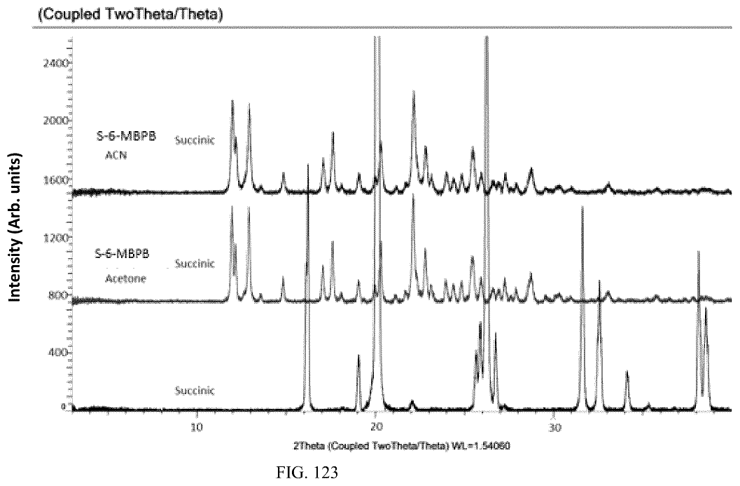

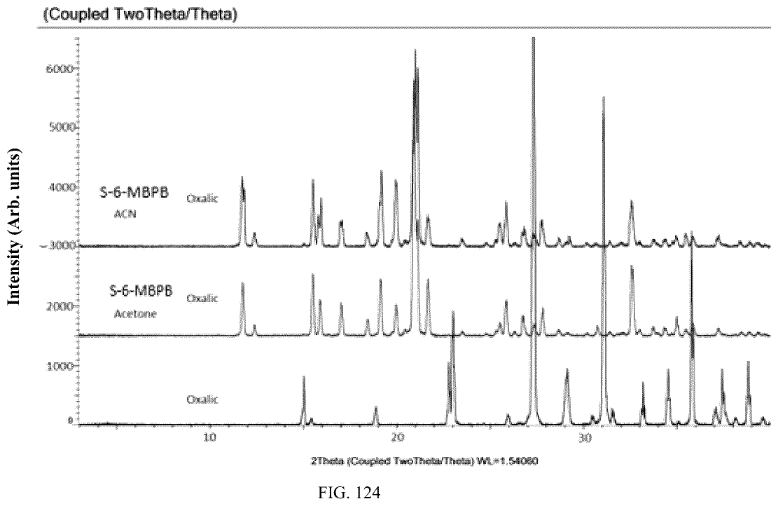

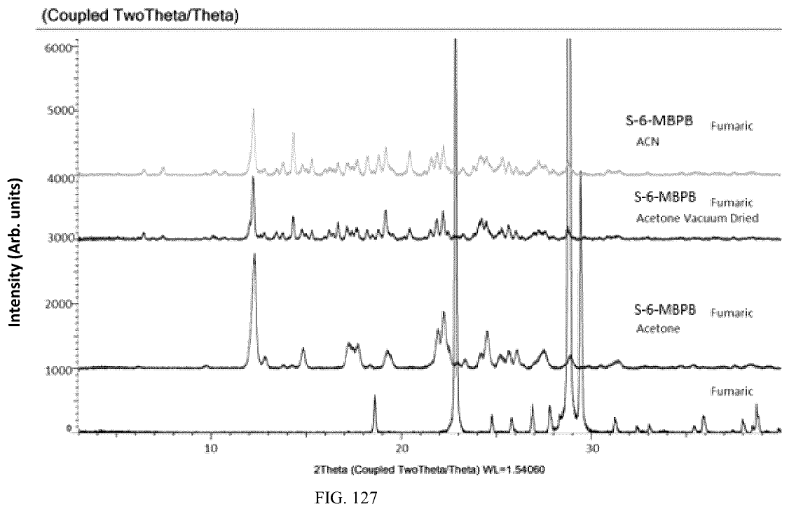

Salt studies of S-6-MBPB were conducted using the conditions in Table 38. The below technique was used to generate the XRPD patterns in FIG. 98, FIG. 102, FIG. 103, FIG. 104, FIG. 105, FIG. 110, FIG. 111, FIG. 112, FIG. 113, FIG. 114, FIG. 115, FIG. 118, FIG. 120, FIG. 122, FIG. 123, FIG. 124, and FIG. 127 (For 40 mg of S-6-MBPB, 1 vol. solvent=40 μL).

TABLE 38
Salt screening experiments of S-6-MBPB

| | | | | PXRD |
|---|---|---|---|---|
| | | | | Result |
| Counter | | | PXRD | (Vacuum |
| ion | Solvent | Procedure/Comments | Result | Dried) |
| HCl | Acetone | 40 mg S-6-MBPB (as freebase oil) + 10 vols. | S-6- | S-6- |
| | | solvent + 1.1:1 molar ratio counterion, stirred at | MBPB | MBPB |
| | | RT, yellow clear solution after ~4 hours stirring, | Pattern | Pattern 1A |
| | | opened for evaporation, gel-like solids after | 1A | |
| | | overnight evaporation, analyzed by PXRD, | | |
| | | placed in vacuum oven at 40° C., analyzed by | | |
| | | PXRD/DSC/TGA | | |
| HBr | Acetone | 40 mg S-6-MBPB (as freebase oil) + 10 vols. | S-6- | S-6- |
| | | solvent + 1.1:1 molar ratio counterion, stirred at | MBPB | MBPB |
| | | RT, yellow clear solution after ~4 hours stirring, | Pattern | Pattern 2A |
| | | opened for evaporation, gel after overnight | 2A | |
| | | evaporation, formed glass-like solids after gel | | |
| | | was disturbed with a spatula, analyzed by | | |
| | | PXRD, placed in vacuum oven at 40° C., | | |
| | | analyzed by PXRD/DSC/TGA | | |
| H₂SO₄ | Acetone | 40 mg S-6-MBPB (as freebase oil) + 10 vols. | N/A | N/A |
| | | solvent + 1.1:1 molar ratio counterion, stirred at | | |
| | | RT, yellow clear solution after ~4 hours stirring, | | |
| | | opened for evaporation, wet gel after 2 days of | | |
| | | evaporation, increased temp. to 40° C., sticky gel | | |
| | | after overnight evaporation, added 5 vols. 1,4- | | |
| | | Dioxane, stirred at 40° C., dark brown gel-like | | |
| | | solids suspended in solvent after ~4 hours, dark | | |
| | | brown gel-like solids suspended in solvent after | | |
| | | 4 days, terminated | | |
| H₃PO₄ | Acetone | 40 mg S-6-MBPB (as freebase oil) + 10 vols. | N/A | N/A |
| | | solvent + 1.1:1 molar ratio counterion, stirred at | | |
| | | RT, yellow clear solution after ~4 hours stirring, | | |
| | | opened for evaporation, wet gel after 2 days of | | |
| | | evaporation, increased temp. to 40° C., sticky gel | | |
| | | after overnight evaporation, added 5 vols. 1,4- | | |
| | | Dioxane, stirred at 40° C., cloudy solution after | | |
| | | ~4 hours, opened for evaporation, sticky gel after | | |
| | | overnight evaporation, added 5 vols. chloroform, | | |
| | | stirred at 40° C., gel-like solids suspended in | | |
| | | solvent after ~3 hours, gel-like solids suspended | | |
| | | in solvent after 3 days, terminated | | |
| HNO₃ | Acetone | 40 mg S-6-MBPB (as freebase oil) + 10 vols. | S-6- | S-6- |
| | | solvent + 1.1:1 molar ratio counterion, stirred at | MBPB | MBPB |
| | | RT, yellow clear solution after ~4 hours stirring, | Pattern | Pattern 5A |
| | | opened for evaporation, gel after overnight | 5A | |
| | | evaporation, formed glass-like solids after gel | | |
| | | was disturbed with a spatula, analyzed by | | |
| | | PXRD, placed in vacuum oven at 40° C., | | |
| | | analyzed by PXRD/DSC/TGA | | |
| Methane | Acetone | 40 mg S-6-MBPB (as freebase oil) + 10 vols. | N/A | N/A |
| sulfonic | | solvent + 1.1:1 molar ratio counterion, stirred at | | |
| | | RT, yellow clear solution after ~4 hours stirring, | | |
| | | opened for evaporation, wet gel after 2 days of | | |
| | | evaporation, increased temp. to 40° C., sticky gel | | |
| | | after overnight evaporation, added 5 vols. 1,4- | | |
| | | Dioxane, stirred at 40° C., clear solution after ~4 | | |
| | | hours, opened for evaporation, sticky gel after | | |
| | | overnight evaporation, added 5 vols. chloroform, | | |
| | | stirred at 40° C., clear solution after ~3 hours, | | |
| | | opened for evaporation, wet gel after 3 days of | | |
| | | evaporation, terminated | | |
| Tartaric | Acetone | 40 mg S-6-MBPB (as freebase oil) + 10 vols. | S-6- | S-6- |
| | | solvent + 1.1:1 molar ratio counterion (stock | MBPB | MBPB |
| | | soln. in water), stirred at RT, yellow clear | Pattern | Pattern 7A |
| | | solution after ~4 hours stirring, opened for | 7A | |
| | | evaporation, clear solution after 2 days of | | |
| | | evaporation, increased temp. to 40° C., sticky gel | | |
| | | after 2 days of evaporation, added 5 vols. 1,4- | | |
| | | Dioxane, stirred at 40° C., cloudy solution after | | |
| | | ~3 hours, opened for evaporation, tan solids after | | |
| | | 3 days of evaporation, analyzed by PXRD, | | |
| | | placed in the vacuum oven at 40° C., analyzed by | | |
| | | PXRD/DSC/TGA | | |
| Succinic | Acetone | 40 mg S-6-MBPB (as freebase oil) + 10 vols. | S-6- | S-6- |
| | | solvent + 1.1:1 molar ratio counterion (stock | MBPB | MBPB |
| | | soln. in water), stirred at RT, yellow clear | Pattern | Pattern 8A |
| | | solution after ~4 hours stirring, opened for | 8A | |
| | | evaporation, clear solution after 2 days of | | |
| | | evaporation, increased temp. to 40° C., sticky gel | | |
| | | after 2 days of evaporation, added 5 vols. 1,4- | | |
| | | Dioxane, stirred at 40° C., clear solution after ~3 | | |
| | | hours, opened for evaporation, tan solids after 3 | | |
| | | days of evaporation, analyzed by PXRD, placed | | |
| | | in the vacuum oven at 40° C., analyzed by | | |
| | | PXRD/DSC/TGA | | |
| Oxalic | Acetone | 40 mg S-6-MBPB (as freebase oil) + 10 vols. | S-6- | S-6- |
| | | solvent + 1.1:1 molar ratio counterion (stock | MBPB | MBPB |
| | | soln. in water), stirred at RT, yellow clear | Pattern | Pattern 9A |
| | | solution after ~4 hours stirring, opened for | 9A | |
| | | evaporation, clear solution after 2 days of | | |
| | | evaporation, increased temp. to 40° C., formed | | |
| | | tan solids after 2 days of evaporation, analyzed | | |
| | | by PXRD, placed in the vacuum oven at 40° C., | | |
| | | analyzed by PXRD/DSC/TGA | | |
| Maleic | Acetone | 40 mg S-6-MBPB (as freebase oil) + 10 vols. | N/A | N/A |
| | | solvent + 1.1:1 molar ratio counterion (stock | | |
| | | soln. in water), stirred at RT, yellow clear | | |
| | | solution after ~4 hours stirring, opened for | | |
| | | evaporation, clear solution after 2 days of | | |
| | | evaporation at RT, increased temp. to 40° C., | | |
| | | sticky gel after 2 days of evaporation, added 5 | | |
| | | vols. 1,4-Dioxane, stirred at 40° C., clear solution | | |
| | | after ~3 hours, opened for evaporation, sticky gel | | |
| | | after 3 days, terminated | | |
| Malic | Acetone | 40 mg S-6-MBPB (as freebase oil) + 10 vols. | N/A | N/A |
| | | solvent + 1.1:1 molar ratio counterion (stock | | |
| | | soln. in water), stirred at RT, yellow clear | | |
| | | solution after ~4 hours stirring, opened for | | |
| | | evaporation, clear solution after 2 days of | | |
| | | evaporation at RT, increased temp. to 40° C., | | |
| | | sticky gel after 2 days of evaporation, added 5 | | |
| | | vols. 1,4-Dioxane, stirred at 40° C., clear solution | | |
| | | after ~3 hours, opened for evaporation, sticky gel | | |
| | | after 3 days, added 5 vols. chloroform, stirred at | | |
| | | 40° C., oil phase suspended in solvent after ~3 | | |
| | | hours, oil phase suspended in solvent after | | |
| | | overnight stirring, terminated | | |
| Citric | Acetone | 40 mg S-6-MBPB (as freebase oil) + 10 vols. | N/A | N/A |
| | | solvent + 1.1:1 molar ratio counterion (stock | | |
| | | soln. in water), stirred at RT, yellow clear | | |
| | | solution after ~4 hours stirring, opened for | | |
| | | evaporation, clear solution after 2 days of | | |
| | | evaporation at RT and 2 days at 40° C., left | | |
| | | evaporating at 40° C., solvent spillage upon | | |
| | | observations, sticky gel-like solids after 3 days, | | |
| | | terminated | | |
| Fumaric | Acetone | 40 mg S-6-MBPB (as freebase oil) + 10 vols. | S-6- | S-6- |
| | | solvent + 1.1:1 molar ratio counterion (stock | MBPB | MBPB |
| | | soln. in water), stirred at RT, yellow clear | Pattern | Pattern |
| | | solution after ~4 hours stirring, opened for | 13A | 13A |
| | | evaporation, glass-like solids after 2 days of | | |
| | | evaporation, analyzed by PXRD, placed in | | |
| | | vacuum oven at 40° C., analyzed by | | |
| | | PXRD/DSC/TGA | | |
| Benzoic | Acetone | 40 mg S-6-MBPB (as freebase oil) + 10 vols. | N/A | N/A |
| | | solvent + 1.1:1 molar ratio counterion (stock | | |
| | | soln. in water), stirred at RT, yellow clear | | |
| | | solution after ~4 hours stirring, opened for | | |
| | | evaporation, gel after 2 days of evaporation, | | |
| | | added 5 vols. 1,4-Dioxane, stirred at RT, clear | | |
| | | solution after ~4 hours, increased temp. to 40° C., | | |
| | | opened for evaporation, gel after overnight | | |
| | | evaporation, added 5 vols. chloroform, stirred at | | |
| | | 40° C., clear solution after ~4 hours, opened for | | |
| | | evaporation at 40° C., gel after overnight | | |
| | | evaporation, terminated | | |
| Salicylic | Acetone | 40 mg S-6-MBPB (as freebase oil) + 10 vols. | N/A | N/A |
| | | solvent + 1.1:1 molar ratio counterion (stock | | |
| | | soln. in water), stirred at RT, yellow clear | | |
| | | solution after ~4 hours stirring, opened for | | |
| | | evaporation, gel after 2 days of evaporation, | | |
| | | added 5 vols. 1,4-Dioxane, stirred at RT, clear | | |
| | | solution after ~4 hours, increased temp. to 40° C., | | |
| | | opened for evaporation, gel after overnight | | |
| | | evaporation, added 5 vols. chloroform, stirred at | | |
| | | 40° C., clear solution after ~4 hours, opened for | | |
| | | evaporation at 40° C., gel after overnight | | |
| | | evaporation, terminated | | |

### EXAMPLE 43 Salt Study Experiments of S-6-MBPB Pure Enantiomer in Me:Water

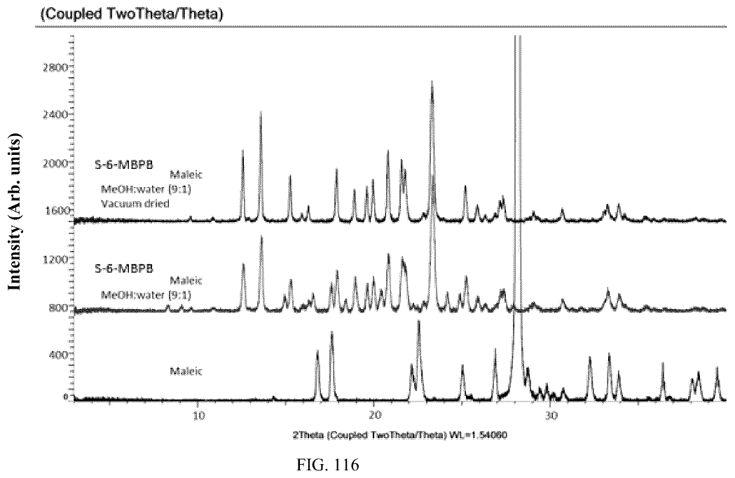

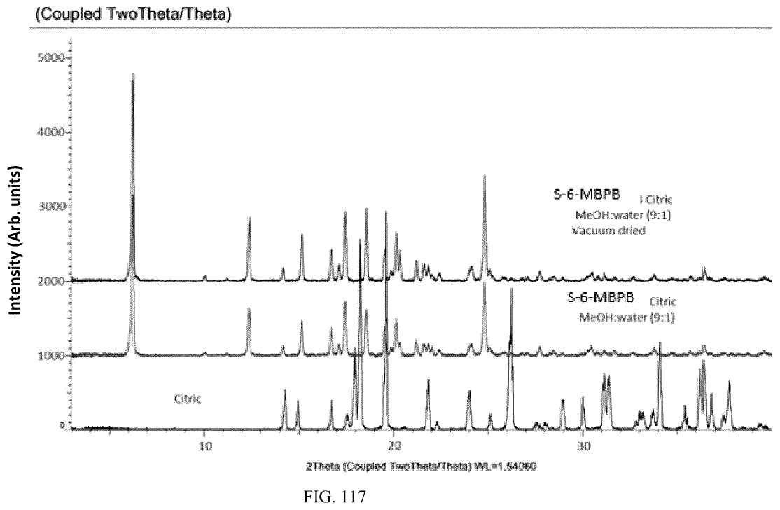

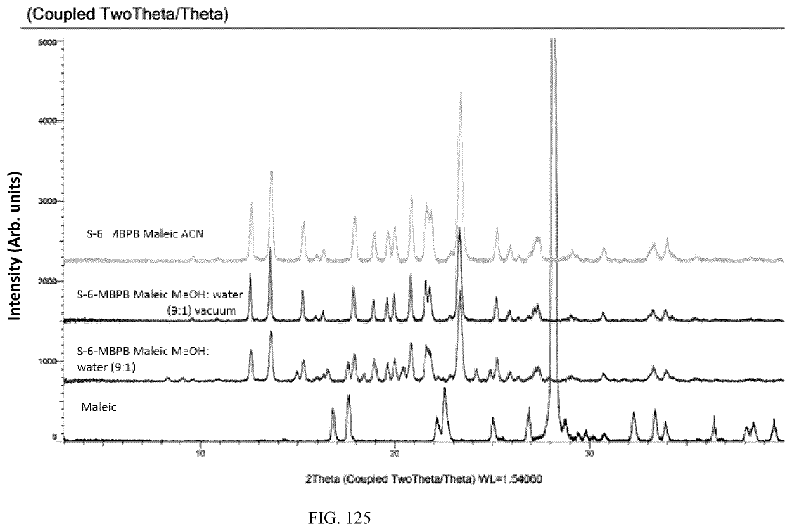

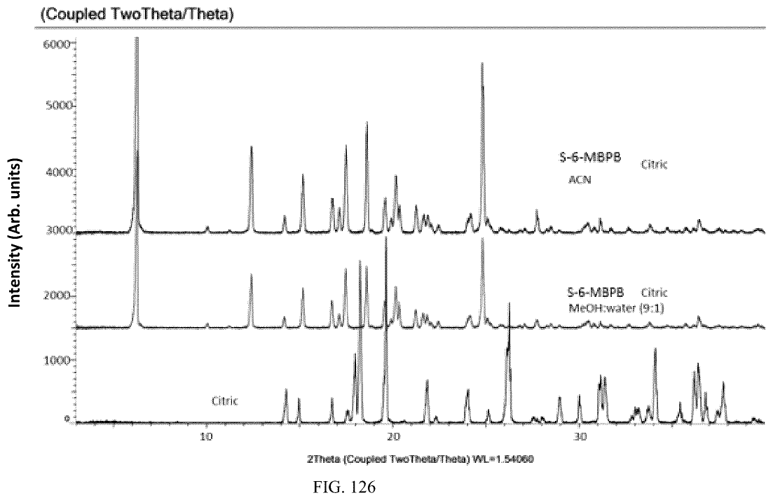

Salt studies of S-6-MBPB were conducted using the conditions in Table 39. The below technique was used to generate the XRPD patterns in FIG. 108, FIG. 110, FIG. 111, FIG. 112, FIG. 113, FIG. 115, FIG. 116, FIG. 117, FIG. 118, FIG. 125, and FIG. 126 (For 40 mg of S-6-MBPB, 1 vol. solvent=40 μl).

TABLE 39
Salt screening experiments of S-6-MBPB

| | | | | PXRD |
|---|---|---|---|---|
| | | | | Result |
| Counter | | | PXRD | (Vacuum |
| ion | Solvent | Procedure/Comments | Result | Dried) |
| HCl | MeOH: | 40 mg S-6-MBPB (as freebase oil) + 10 vols. | S-6- | N/A |
| | water | solvent + 1.1:1 molar ratio counterion, stirred at | MBPB | |
| | 9:1 | RT, yellow clear solution after ~4 hours stirring, | Pattern | |
| | | opened for evaporation, glass-like solids after 2 | 1A | |
| | | days of evaporation, analyzed by PXRD, placed | | |
| | | in vacuum oven at 40° C. | | |
| HBr | MeOH: | 40 mg S-6-MBPB (as freebase oil) + 10 vols. | S-6- | N/A |
| | water | solvent + 1.1:1 molar ratio counterion, stirred at | MBPB | |
| | 9:1 | RT, yellow clear solution after ~4 hours stirring, | Pattern | |
| | | opened for evaporation, gel after 2 days of | 2A | |
| | | evaporation, added 5 vols. 1,4-Dioxane to | | |
| | | reslurry, stirred at RT, thin slurry after ~4 hours, | | |
| | | light brown/orange slurry after overnight stirring, | | |
| | | centrifuged to recover solids, analyzed by PXRD, | | |
| | | placed in vacuum oven at 40° C. | | |
| H₂SO₄ | MeOH: | 40 mg S-6-MBPB (as freebase oil) + 10 vols. | N/A | N/A |
| | water | solvent + 1.1:1 molar ratio counterion, stirred at | | |
| | 9:1 | RT, yellow clear solution after ~4 hours stirring, | | |
| | | opened for evaporation, wet gel after 2 days of | | |
| | | evaporation, increased temp. to 40° C., sticky gel | | |
| | | after overnight evaporation, added 5 vols. | | |
| | | chloroform, stirred at 40° C., dark brown gel-like | | |
| | | solids suspended in solvent after ~4 hours, dark | | |
| | | brown gel-like solids suspended in solvent after 4 | | |
| | | days, terminated | | |
| H₃PO₄ | MeOH: | 40 mg S-6-MBPB (as freebase oil) + 10 vols. | N/A | N/A |
| | water | solvent + 1.1:1 molar ratio counterion, stirred at | | |
| | 9:1 | RT, yellow clear solution after ~4 hours stirring, | | |
| | | opened for evaporation, clear solution after 2 days | | |
| | | of evaporation, increased temp. to 40° C., wet gel | | |
| | | after 5 days of evaporation, terminated | | |
| HNO₃ | MeOH: | 40 mg S-6-MBPB (as freebase oil) + 10 vols. | S-6- | N/A |
| | water | solvent + 1.1:1 molar ratio counterion, stirred at | MBPB | |
| | 9:1 | RT, yellow clear solution after ~4 hours stirring, | Pattern | |
| | | opened for evaporation, gel after overnight | 5A | |
| | | evaporation, formed glass-like solids after gel | | |
| | | was disturbed with a spatula, analyzed by PXRD, | | |
| | | placed in vacuum oven at 40° C. | | |
| Methane | MeOH: | 40 mg S-6-MBPB (as freebase oil) + 10 vols. | N/A | N/A |
| sulfonic | water | solvent + 1.1:1 molar ratio counterion, stirred at | | |
| | 9:1 | RT, yellow clear solution after ~4 hours stirring, | | |
| | | opened for evaporation, wet gel after 2 days of | | |
| | | evaporation, increased temp. to 40° C., sticky gel | | |
| | | after overnight evaporation, added 5 vols. | | |
| | | chloroform, stirred at 40° C., clear solution after | | |
| | | ~4 hours, opened for evaporation at 40° C., sticky | | |
| | | gel after overnight evaporation, added 5 vols. 1,4- | | |
| | | Dioxane, stirred at 40° C., clear solution after ~3 | | |
| | | hours, opened for evaporation, wet gel after 3 | | |
| | | days, terminated | | |
| Tartaric | MeOH: | 40 mg S-6-MBPB (as freebase oil) + 10 vols. | S-6- | N/A |
| | water | solvent + 1.1:1 molar ratio counterion (stock soln. | MBPB | |
| | 9:1 | in water), stirred at RT, yellow clear solution after | Pattern | |
| | | ~4 hours stirring, opened for evaporation, clear | 7A w/ | |
| | | solution after 2 days of evaporation, increased | minor | |
| | | temp to. 40° C., sticky gel after 2 days of | Tartaric | |
| | | evaporation, added 5 vols. chloroform, stirred at | Acid | |
| | | 40° C., gel-like solids suspended in solvent afetr | peaks at | |
| | | ~3 hours, wet solids after 3 days, analyzed by | 10.0°, | |
| | | PXRD, placed in vacuum oven at 40° C. | 19.7°. | |
| | | | 21.8°, | |
| | | | 30.1°, | |
| | | | and | |
| | | | 32.8° | |
| Succinic | MeOH: | 40 mg S-6-MBPB (as freebase oil) + 10 vols. | N/A | N/A |
| | water | solvent + 1.1:1 molar ratio counterion (stock soln. | | |
| | 9:1 | in water), stirred at RT, yellow clear solution after | | |
| | | ~4 hours stirring, opened for evaporation, clear | | |
| | | solution after 2 days of evaporation, increased | | |
| | | temp to. 40° C., sticky gel after 2 days of | | |
| | | evaporation, added 5 vols. chloroform, stirred at | | |
| | | 40° C., clear solution after ~3 hours, opened for | | |
| | | evaporation, sticky gel after 3 days, terminated | | |
| Oxalic | MeOH: | 40 mg S-6-MBPB (as freebase oil) + 10 vols. | S-6- | N/A |
| | water | solvent + 1.1:1 molar ratio counterion (stock soln. | MBPB | |
| | 9:1 | in water), stirred at RT, yellow clear solution after | Pattern | |
| | | ~4 hours stirring, opened for evaporation, clear | 9A | |
| | | solution after 2 days of evaporation, increased | | |
| | | temp. to 40° C., tan solids after 2 days of | | |
| | | evaporation, analyzed by PXRD, placed in the | | |
| | | vacuum oven at 40° C. | | |
| Maleic | MeOH: | 40 mg S-6-MBPB (as freebase oil) + 10 vols. | S-6- | S-6- |
| | water | solvent + 1.1:1 molar ratio counterion (stock soln. | MBPB | MBPB |
| | 9:1 | in water), stirred at RT, yellow clear solution after | Pattern | Pattern |
| | | ~4 hours stirring, opened for evaporation, clear | 10A | 10A with |
| | | solution after 2 days of evaporation, increased | | missing |
| | | temp to. 40° C., sticky gel after 2 days of | | peaks at |
| | | evaporation, added 5 vols. chloroform, stirred at | | 17.6°, |
| | | 40° C., clear solution after ~3 hours, opened for | | 18.4°, |
| | | evaporation, sticky gel after 3 days of | | 20.4°, |
| | | evaporation, added 5 vols. 1,4-Dioxane, stirred at | | 24.2°, |
| | | 40° C., wet solids after solvent addition, analyzed | | 24.9° |
| | | by PXRD, placed in the vacuum oven at 40° C., | | |
| | | analyzed by PXRD/DSC/TGA | | |
| Malic | MeOH: | 40 mg S-6-MBPB (as freebase oil) + 10 vols. | N/A | N/A |
| | water | solvent + 1.1:1 molar ratio counterion (stock soln. | | |
| | 9:1 | in water), stirred at RT, yellow clear solution after | | |
| | | ~4 hours stirring, opened for evaporation, clear | | |
| | | solution after 2 days of evaporation, increased | | |
| | | temp to. 40° C., sticky gel after 2 days of | | |
| | | evaporation, added 5 vols. chloroform, stirred at | | |
| | | 40° C., oiled out phase suspended in solvent after | | |
| | | ~3 hours, thick clear solution after 3 days, opened | | |
| | | for evaporation, sticky gel after overnight | | |
| | | evaporation, terminated | | |
| Citric | MeOH: | 40 mg S-6-MBPB (as freebase oil) + 10 vols. | S-6- | S-6- |
| | water | solvent + 1.1:1 molar ratio counterion (stock soln. | MBPB | MBPB |
| | 9:1 | in water), stirred at RT, yellow clear solution after | Pattern | Pattern |
| | | ~4 hours stirring, opened for evaporation, clear | 12A | 12A |
| | | solution after 2 days of evaporation, increased | | |
| | | temp to. 40° C., sticky gel after 2 days of | | |
| | | evaporation, added 5 vols. 1,4-Dioxane, stirred at | | |
| | | 40° C., clear solution after ~3 hours, opened for | | |
| | | evaporation, sticky gel after 3 days, added 5 vols. | | |
| | | chloroform, stirred at 40° C., suspended gels in a | | |
| | | cloudy solution after ~3 hours, wet solids after | | |
| | | overnight stirring, analyzed by PXRD, placed in | | |
| | | the vacuum oven at 40° C., analyzed by | | |
| | | PXRD/DSC/TGA | | |
| Fumaric | MeOH: | 40 mg S-6-MBPB (as freebase oil) + 10 vols. | S-6- | N/A |
| | water | solvent + 1.1:1 molar ratio counterion (stock soln. | MBPB | |
| | 9:1 | in water), stirred at RT, yellow clear solution after | Pattern | |
| | | ~4 hours stirring, opened for evaporation, gel | 13A | |
| | | after 2 days of evaporation, added 5 vols. 1,4- | | |
| | | Dioxane, stirred at RT, clear solution after ~4 | | |
| | | hours, opened for evaporation at 40° C., light tan | | |
| | | solids after overnight evaporation, analyzed by | | |
| | | PXRD, placed in the vacuum oven at 40° C. | | |
| Benzoic | MeOH: | 40 mg S-6-MBPB (as freebase oil) + 10 vols. | N/A | N/A |
| | water | solvent + 1.1:1 molar ratio counterion (stock soln. | | |
| | 9:1 | in water), stirred at RT, yellow clear solution after | | |
| | | ~4 hours stirring, opened for evaporation, wet gel | | |
| | | after 2 days of evaporation, increased temp. to 40° | | |
| | | C., sticky gel after overnight evaporation, added | | |
| | | 5 vols. chloroform, stirred at 40° C., clear solution | | |
| | | after ~4 hours, opened for evaporation, sticky gel | | |
| | | after overnight evaporation, added 5 vols. 1,4- | | |
| | | Dioxane, stirred at 40° C., clear solution after ~3 | | |
| | | hours, opened for evaporation, sticky gel after 3 | | |
| | | days of evaporation, terminated | | |
| Salicylic | MeOH: | 40 mg S-6-MBPB (as freebase oil) + 10 vols. | N/A | N/A |
| | water | solvent + 1.1:1 molar ratio counterion (stock soln. | | |
| | 9:1 | in water), stirred at RT, yellow clear solution after | | |
| | | ~4 hours stirring, opened for evaporation, wet gel | | |
| | | after 2 days of evaporation, increased temp. to 40° | | |
| | | C., sticky gel after overnight evaporation, added | | |
| | | 5 vols. chloroform, stirred at 40° C., clear solution | | |
| | | after ~4 hours, opend for evaporation, sticky gel | | |
| | | after overnight evaporation, added 5 vols. 1,4- | | |
| | | Dioxane, stirred at 40° C., clear solution after ~3 | | |
| | | hours, opened for evaporation, sticky gel after 3 | | |
| | | days of evaporation, terminated | | |

### EXAMPLE 44 Salt Study Experiments of S-6-MBPB Pure Enantiomer in ACN

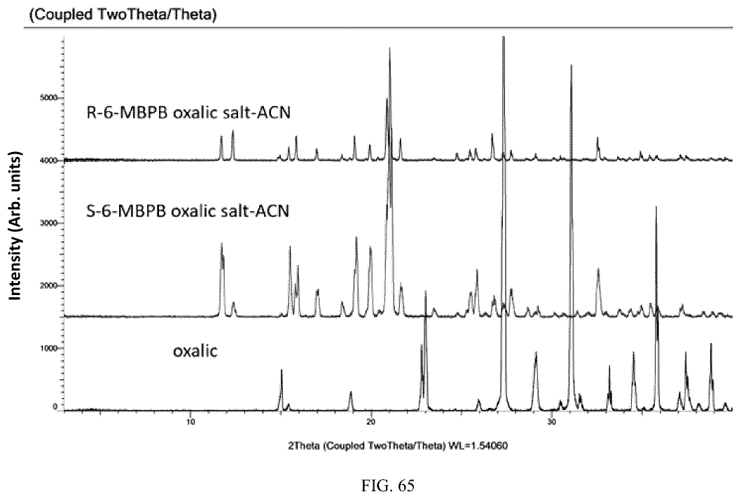

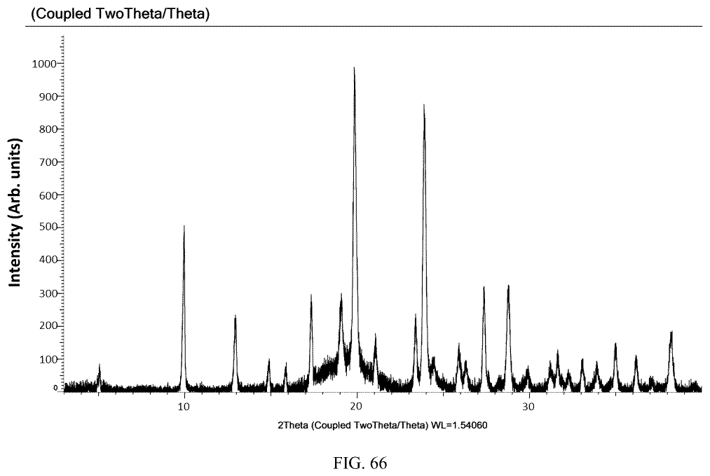

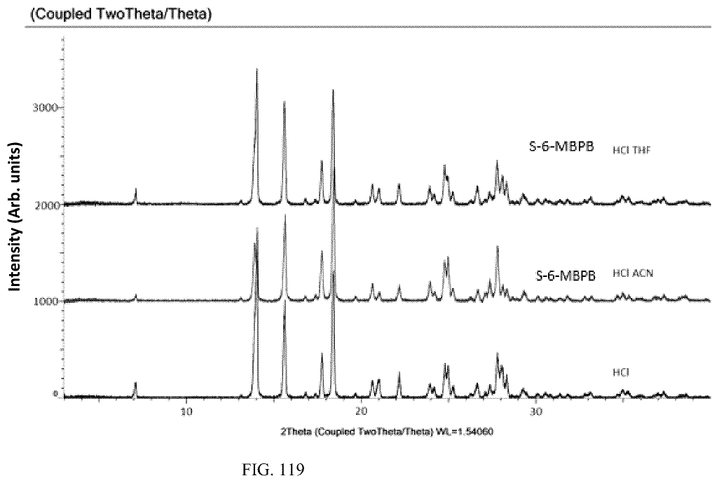

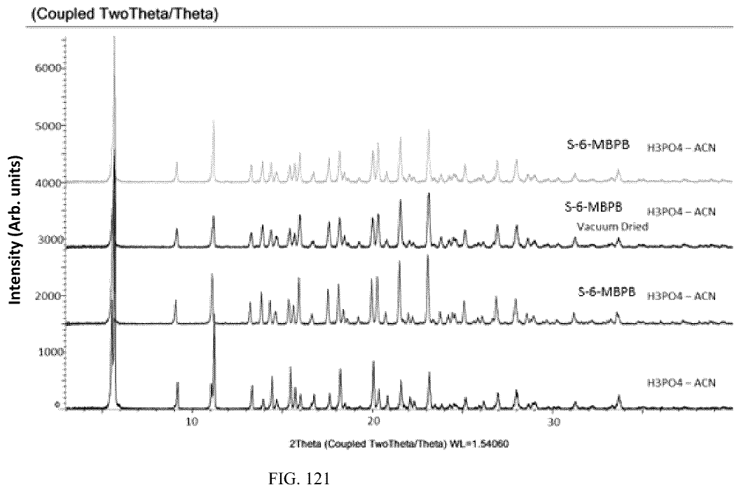

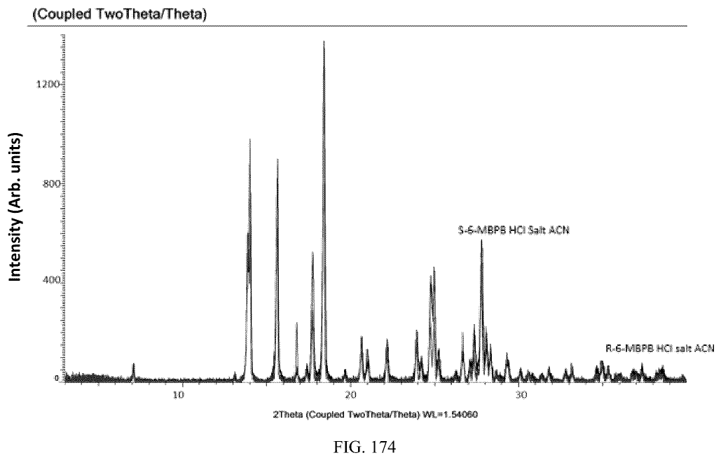

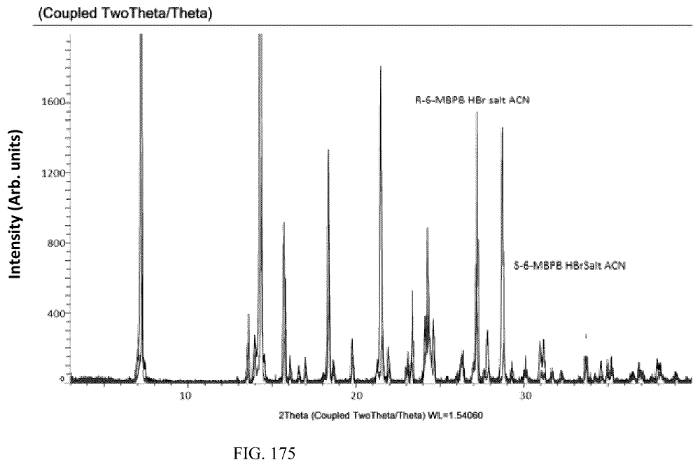

Salt studies of S-6-MBPB were conducted using the conditions in Table 40. The below technique was used to generate the XRPD patterns in FIG. 100, FIG. 101, FIG. 106, FIG. 107, FIG. 109, FIG. 119, FIG. 120, FIG. 121, FIG. 122, FIG. 123, FIG. 124, FIG. 125, FIG. 126, FIG. 127, FIG. 174, FIG. 175, and FIG. 65 (For 40 mg of S-6-MBPB, 1 vol. solvent=40 μL).

TABLE 40
Salt screening experiments of S-6-MBPB

| | | | | PXRD Result |
|---|---|---|---|---|
| Counterion | Solvent | Procedure/Comments | PXRD Result | (Vacuum Dried) |
| HCl | ACN | 40 mg S-6-MBPB (as freebase oil) + 5 vols. | S-6-MBPB | N/A |
| | | solvent + 1.1:1 molar ratio counterion, stirred at | Pattern 1A | |
| | | 40° C., clear yellow solution after~45 minutes, | | |
| | | opened for evaporation, light tan solids after | | |
| | | overnight evaporation, analyzed by PXRD, | | |
| | | placed in the vacuum oven at 40° C. | | |
| HBr | ACN | 40 mg S-6-MBPB (as freebase oil) + 5 vols. | S-6-MBPB | N/A |
| | | solvent + 1.1:1 molar ratio counterion, stirred at | Pattern 2A | |
| | | 40° C., clear yellow solution after~45 minutes, | (more cryst) | |
| | | opened for evaporation, dark glassy solids after | | |
| | | overnight evaporation, analyzed by PXRD, | | |
| | | placed in the vacuum oven at 40° C., analyzed | | |
| | | by NMR | | |
| H₂SO₄ | ACN | 40 mg S-6-MBPB (as freebase oil) + 5 vols. | N/A | N/A |
| | | solvent + 1.1:1 molar ratio counterion, stirred at | | |
| | | 40° C., clear yellow solution after~45 minutes, | | |
| | | opened for evaporation, sticky gel after | | |
| | | overnight evaporation, added 5 vols. DCM, | | |
| | | stirred at 40° C., oily solution after~4 hours, oily | | |
| | | solution after overnight stirring, opened for | | |
| | | evaporation, wet gel after~5 hours, gel after 3 | | |
| | | days of evaporation, terminated | | |
| H₃PO₄ | ACN | 40 mg S-6-MBPB (as freebase oil) + 5 vols. | Similar to | N/A |
| | | solvent + 1.1:1 molar ratio counterion, stirred at | S-6-MBPB | |
| | | 40° C., wet tan solids sitting in solvent after~45 | Pattern 4A | |
| | | minutes, wet tan solids sitting in solvent after | with minor | |
| | | overnight stirring, solvent pipetted out, | shoulders at | |
| | | analyzed solids by PXRD | 5.5° and 11.1° | |
| HNO₃ | ACN | 40 mg S-6-MBPB (as freebase oil) + 5 vols. | N/A | N/A |
| | | solvent + 1.1:1 molar ratio counterion, stirred at | | |
| | | 40° C., clear yellow solution after~45 minutes, | | |
| | | opened for evaporation, sticky gel after | | |
| | | overnight evaporation, added 5 vols. 1,4- | | |
| | | Dioxane, stirred at 40° C., dark orange clear | | |
| | | solution after~4 hours, opened for evaporation, | | |
| | | gel after overnight evaporation, added 5 vols. | | |
| | | chloroform, dark orange clear solution after~5 | | |
| | | hours, opened for evaporation, gel after 3 days | | |
| | | of evaporation, terminated | | |
| Methane | ACN | 40 mg S-6-MBPB (as freebase oil) + 5 vols. | N/A | N/A |
| sulfonic | | solvent + 1.1:1 molar ratio counterion, stirred at | | |
| | | 40° C., clear yellow solution after~45 minutes, | | |
| | | opened for evaporation, sticky gel after | | |
| | | overnight evaporation, added 5 vols. DCM, | | |
| | | stirred at 40° C., clear yellow solution after~4 | | |
| | | hours, opened for evaporation, gel after | | |
| | | overnight evaporation, terminated | | |
| Tartaric | ACN | 40 mg S-6-MBPB (as freebase oil) + 5 vols. | S-6-MBPB | N/A |
| | | solvent + 1.1:1 molar ratio counterion (stock | Pattern 7A | |
| | | soln. in water), stirred at 40° C., clear yellow | | |
| | | solution after~45 minutes, opened for | | |
| | | evaporation, gel after 2 days of evaporation, | | |
| | | added 5 vols. DCM, stirred at 40° C., gel sitting | | |
| | | in solvent after~5 hours, gel sitting in solvent | | |
| | | after 3 days, opened for evaporation, gel after~5 | | |
| | | hours, added 5 vols. EtOAc, stirred at 40° C., | | |
| | | wet solids in clear solvent after overnight | | |
| | | stirring, opened for solvent evaporation, dry | | |
| | | solids after~3 hours, analyzed by PXRD, | | |
| | | placed in vacuum oven at 40° C. | | |
| Succinic | ACN | 40 mg S-6-MBPB (as freebase oil) + 5 vols. | S-6-MBPB | N/A |
| | | solvent + 1.1:1 molar ratio counterion (stock | Pattern 8A | |
| | | soln. in water), stirred at 40° C., clear yellow | | |
| | | solution after~45 minutes, opened for | | |
| | | evaporation, clear solution after overnight | | |
| | | evaporation, gel after 2 days of evaporation, | | |
| | | added 5 vols. DCM, stirred at 40° C., clear | | |
| | | orange solution after~5 hours stirring, opened | | |
| | | for evaporation, gel after 3 days of evaporation, | | |
| | | added 5 vols. EtOAc, stirred at 40° C., wet solids | | |
| | | after~5 hours, opened for evaporation, dry off- | | |
| | | white solids after overnight evaporation, | | |
| | | analyzed by PXRD, placed in vacuum oven at | | |
| | | 40° C. | | |
| Oxalic | ACN | 40 mg S-6-MBPB (as freebase oil) + 5 vols. | S-6-MBPB | N/A |
| | | solvent + 1.1:1 molar ratio counterion (stock | Pattern 9A | |
| | | soln. in water), stirred at 40° C., clear yellow | | |
| | | solution after~45 minutes, opened for | | |
| | | evaporation, clear solution after overnight | | |
| | | evaporation, off-white solids after 2 days of | | |
| | | evaporation, analyzed by PXRD, placed in the | | |
| | | vacuum oven at 40° C. | | |
| Maleic | ACN | 40 mg S-6-MBPB (as freebase oil) + 5 vols. | S-6-MBPB | N/A |
| | | solvent + 1.1:1 molar ratio counterion (stock | Pattern 10A | |
| | | soln. in water), stirred at 40° C., clear yellow | | |
| | | solution after~45 minutes, opened for | | |
| | | evaporation, clear solution after overnight | | |
| | | evaporation, gel after 2 days of evaporation, | | |
| | | added 5 vols. DCM, stirred at 40° C., clear | | |
| | | orange solution after~5 hours stirring, opened | | |
| | | for evaporation, gel after 3 days of evaporation, | | |
| | | added 5 vols. EtOAc, stirred at 40° C., clear | | |
| | | solution after~5 hours, opened for evaporation, | | |
| | | tan solids after overnight evaporation, analyzed | | |
| | | by PXRD, placed in vacuum oven at 40° C. | | |
| Malic | ACN | 40 mg S-6-MBPB (as freebase oil) + 5 vols. | N/A | N/A |
| | | solvent + 1.1:1 molar ratio counterion (stock | | |
| | | soln. in water), stirred at 40° C., clear yellow | | |
| | | solution after~45 minutes, opened for | | |
| | | evaporation, clear solution after overnight | | |
| | | evaporation, gel after 2 days of evaporation, | | |
| | | added 5 vols. DCM, stirred at 40° C., oiled phase | | |
| | | separation after~5 hours, oiled phase separation | | |
| | | after 3 days, opened for evaporation, gel after~5 | | |
| | | hours, terminated | | |
| Citric | ACN | 40 mg S-6-MBPB (as freebase oil) + 5 vols. | S-6-MBPB | N/A |
| | | solvent + 1.1:1 molar ratio counterion (stock | Pattern 12A | |
| | | soln. in water), stirred at 40° C., clear yellow | | |
| | | solution after~45 minutes, opened for | | |
| | | evaporation, solids after 5 days of evaporation, | | |
| | | analyzed by PXRD, placed in vacuum oven at | | |
| | | 40° C. | | |
| Fumaric | ACN | 40 mg S-6-MBPB (as freebase oil) + 5 vols. | S-6-MBPB | S-6-MBPB |
| | | solvent + 1.1:1 molar ratio counterion (stock | Pattern 13B | Pattern 13B |
| | | soln. in water), stirred at 40° C., clear yellow | | |
| | | solution after~45 minutes, opened for | | |
| | | evaporation, sticky gel after overnight | | |
| | | evaporation, formed glass-like solids after gel | | |
| | | was disturbed with a spatula, analyzed by | | |
| | | PXRD, placed in vacuum oven at 40° C., | | |
| | | analyzed by PXRD/DSC/TGA | | |
| Benzoic | ACN | 40 mg S-6-MBPB (as freebase oil) + 5 vols. | N/A | N/A |
| | | solvent + 1.1:1 molar ratio counterion (stock | | |
| | | soln. in water), stirred at 40° C., clear yellow | | |
| | | solution after~45 minutes, opened for | | |
| | | evaporation, sticky gel after overnight | | |
| | | evaporation, added 5 vols. DCM, stirred at 40° | | |
| | | C., clear yellow solution after~4 hours, opened | | |
| | | for evaporation, gel after overnight evaporation, | | |
| | | terminated | | |
| Salicylic | ACN | 40 mg S-6-MBPB (as freebase oil) + 5 vols. | N/A | N/A |
| | | solvent + 1.1:1 molar ratio counterion (stock | | |
| | | soln. in water), stirred at 40° C., clear yellow | | |
| | | solution after~45 minutes, opened for | | |
| | | evaporation, sticky gel after overnight | | |
| | | evaporation, added 5 vols. DCM, stirred at 40° | | |
| | | C., clear yellow solution after~4 hours, opened | | |
| | | for evaporation, gel aftert overnight | | |
| | | evaporation, terminated | | |
| H₂SO₄ | ACN | 40 mg S-6-MBPB (as freebase oil) + 5 vols. | N/A | N/A |
| | | solvent + 0.55:1 molar ratio counterion, stirred | | |
| | | at 40° C., orange oil phase separation after~45 | | |
| | | minutes, orange oil phase separation after | | |
| | | overnight stirring, opened for evaporation, gel | | |
| | | after~4 hours, added 5 vols. EtOAc, stirred at | | |
| | | 40° C., gel sitting in solvent after~2 hours, gel | | |
| | | sitting in solvent after overnight stirring, | | |
| | | opened for evaporation, gel after~6 hours, | | |
| | | terminated | | |
| H₃PO₄ | ACN | 40 mg S-6-MBPB (as freebase oil) + 5 vols. | S-6-MBPB | S-6-MBPB |
| | | solvent + 0.33:1 molar ratio counterion, stirred | Pattern 4A | Pattern 4A |
| | | at 40° C., tan slurry after~45 minutes, slurry | | |
| | | after overnight stirring, centrifuged to recover | | |
| | | solids, analyzed by PXRD, placed in the | | |
| | | vacuum oven 40° C., analyzed by | | |
| | | PXRD/DSC/TGA | | |
| H₃PO₄ | ACN | 40 mg S-6-MBPB (as freebase oil) + 5 vols. | S-6-MBPB | N/A |
| | | solvent + 0.55:1 molar ratio counterion, stirred | Pattern 4A | |
| | | at 40° C., tan slurry after~45 minutes, slurry | | |
| | | after overnight stirring, centrifuged to recover | | |
| | | solids, analyzed by PXRD, placed in the | | |
| | | vacuum oven at 40° C. | | |

### EXAMPLE 45 Salt Study Experiments of S-6-MBPB Pure Enantiomer in THF

Salt studies of S-6-MBPB were conducted using the conditions in Table 41. The below technique was used to generate the XRPD patterns in FIG. 119 (For 40 mg of S-6-MBPB, 1 vol. solvent=40 μL).

TABLE 41
Salt screening experiments of S-6-MBPB

| | | | | PXRD Result |
|---|---|---|---|---|
| Counterion | Solvent | Procedure/Comments | PXRD Result | (Vacuum Dried) |
| HCl | THF | 40 mg S-6-MBPB (as freebase oil) + 5 vols. | S-6-MBPB | N/A |
| | | solvent + 1.1:1 molar ratio counterion, stirred at | Pattern 1A | |
| | | 40° C., clear yellow solution after~45 minutes, | | |
| | | opened for evaporation, dark glassy solids after | | |
| | | overnight evaporation, analyzed by PXRD, | | |
| | | placed in the vacuum oven at 40° C. | | |
| Methane | THF | 40 mg S-6-MBPB (as freebase oil) + 5 vols. | N/A | N/A |
| sulfonic | | solvent + 1.1:1 molar ratio counterion, stirred at | | |
| | | 40° C., clear yellow solution after~45 minutes, | | |
| | | opened for evaporation, sticky gel after | | |
| | | overnight evaporation, added 5 vols. EtOAc, | | |
| | | stirred at 40° C., clear yellow solution after~4 | | |
| | | hours, opened for evaporation, gel after | | |
| | | overnight evaporation, terminated | | |
| Malic | THF | 40 mg S-6-MBPB (as freebase oil) + 5 vols. | N/A | N/A |
| | | solvent + 1.1:1 molar ratio counterion (stock | | |
| | | soln. in water), stirred at 40° C., clear yellow | | |
| | | solution after~45 minutes, opened for | | |
| | | evaporation, gel after 2 days of evaporation, | | |
| | | added 5 vols. EtOAc, stirred at 40° C., gel sitting | | |
| | | in solvent after~5 hours, gel sitting in solvent | | |
| | | after 3 days, opened for evaporation, gel after~5 | | |
| | | hours, terminated | | |
| Benzoic | THF | 40 mg S-6-MBPB (as freebase oil) + 5 vols. | N/A | N/A |
| | | solvent + 1.1:1 molar ratio counterion (stock | | |
| | | soln. in water), stirred at 40° C., clear yellow | | |
| | | solution after~45 minutes, opened for | | |
| | | evaporation, sticky gel after overnight | | |
| | | evaporation, added 5 vols. EtOAc, stirred at 40° | | |
| | | C., clear yellow solution after~4 hours, opened | | |
| | | for evaporation, gel after overnight evaporation, | | |
| | | terminated | | |
| Salicylic | THF | 40 mg S-6-MBPB (as freebase oil) + 5 vols. | N/A | N/A |
| | | solvent + 1.1:1 molar ratio counterion (stock | | |
| | | soln. in water), stirred at 40° C., clear yellow | | |
| | | solution after~45 minutes, opened for | | |
| | | evaporation, sticky gel after overnight | | |
| | | evaporation, added 5 vols. EtOAc, stirred at 40° | | |
| | | C., clear yellow solution after~4 hours, opened | | |
| | | for evaporation, gel after overnight evaporation, | | |
| | | terminated | | |

### EXAMPLE 46 Scale Up of S-5-MBPB Liquid-Liquid Extraction

Scale ups of the S-r-MBPB liquid-liquid extraction expt. were performed to isolate large amounts of the Freebase API from the S-5-MBPB HCl salt. ˜0.5 g API (as HCl salt) plus 36.7 vols. (18.4 mL) of EtOAc were added to NaH soln. in water (5 mg/mL) for 1.1:1 base:salt molar ratio. Organic phase was then recovered, the solvent was allowed to evaporate at RT, and the resulting brown gel was analyzed by NMR. Table 42 describes scale up conditions of liquid-liquid extraction conditions of S-5-MBPB.

TABLE 42
Liquid-liquid extraction scale up experiments of S-5-MBPB

| | Solvent | |
|---|---|---|
| Base | (Density) | Procedure/Comments |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | The top organic layers of S-5-MBPB were pipetted out and combined, |
| | (0.902 g/mL) | concentration estimated as 23.4 mg/mL, concentration closer to 23.1 mg/mL |
| NaOH | EtOAc | The top organic layers of S-5-MBPB were pipetted out and combined, |
| | (0.902 g/mL) | concentration estimated as 23.4 mg/mL, concentration closer to 23.1 mg/mL, |
| | | analyzed by NMR |

### EXAMPLE 47. Salt Study Experiments of S-5-MBPB Pure Enantiomer in Acetone

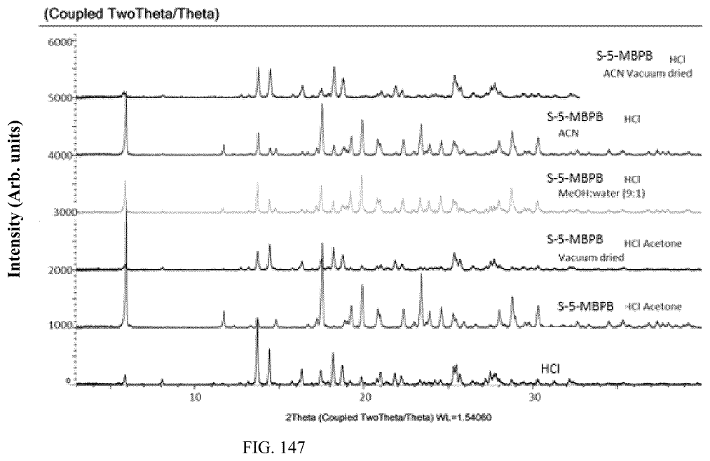

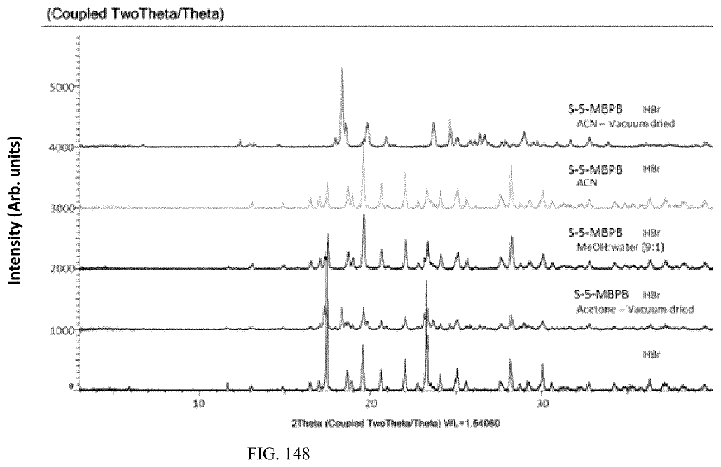

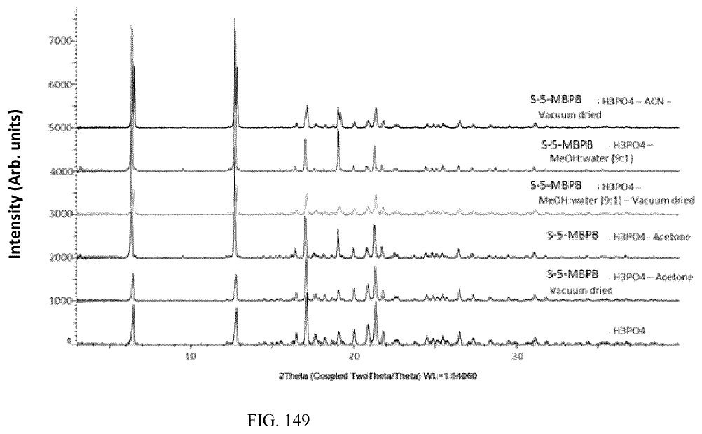

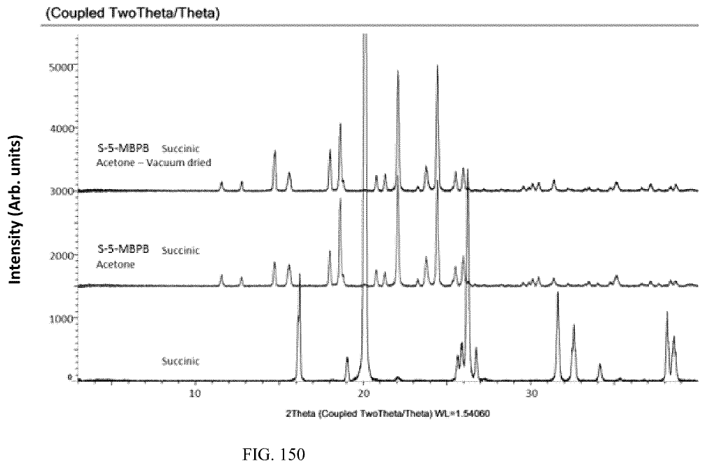

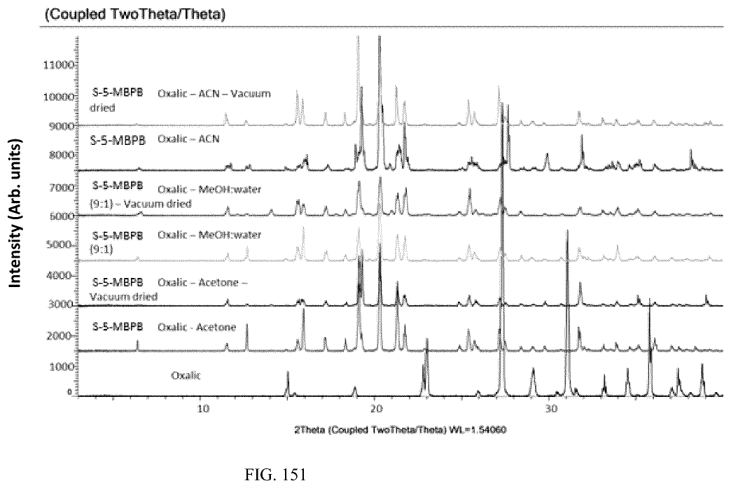

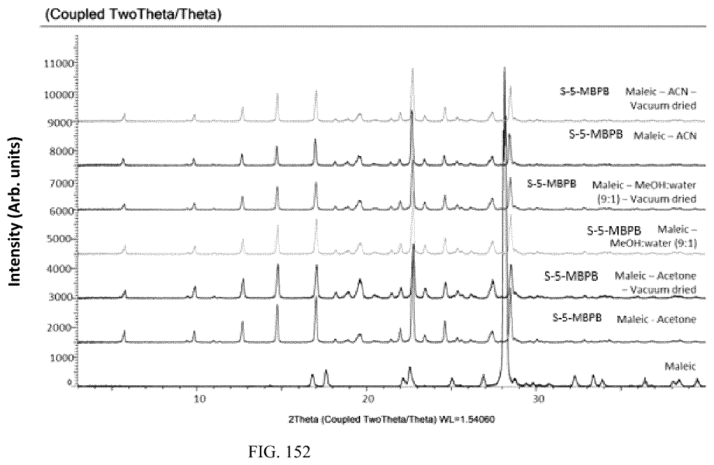

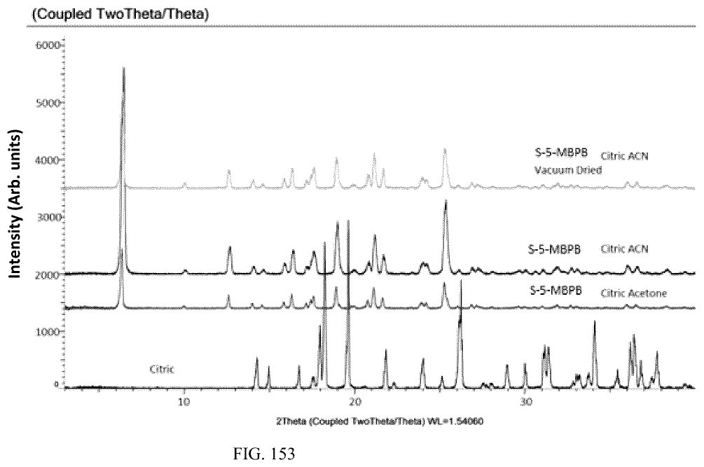

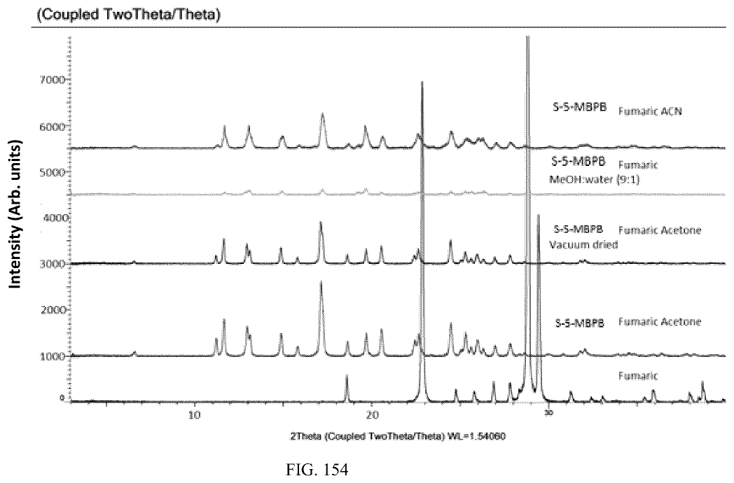

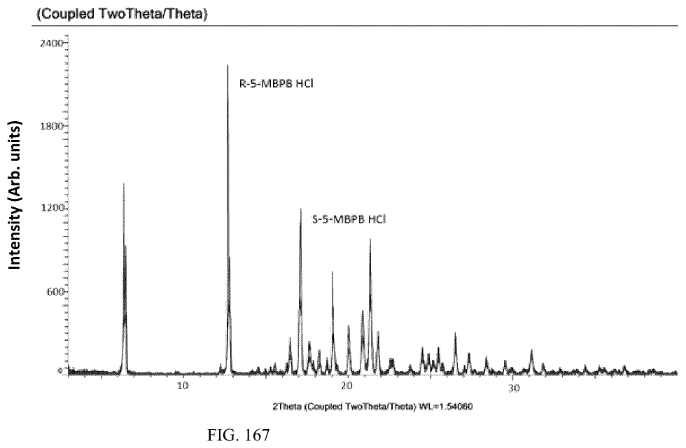

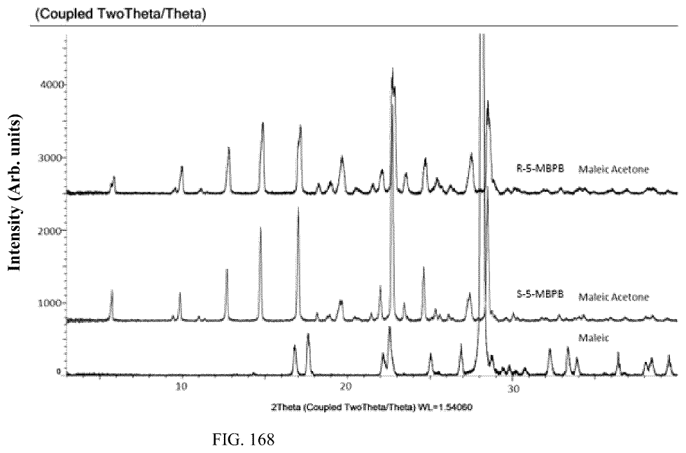

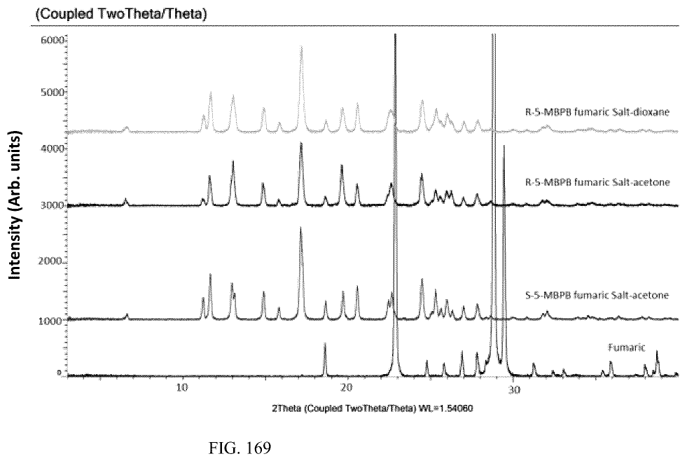

Salt studies of S-5-MBPB were conducted using the conditions in Table 43. The below technique was used to generate the XRPD patterns in FIG. 140, FIG. 142, FIG. 143, FIG. 144, FIG. 146, FIG. 147, FIG. 148, FIG. 149, FIG. 150, FIG. 151, FIG. 152, FIG. 153, FIG. 154, FIG. 167, FIG. 168, and FIG. 169 (For 40 mg of S-5-MBPB, 1 vol. solvent=40 μL).

TABLE 43
Salt screening experiments of S-5-MBPB

| | | | | PXRD Result |
|---|---|---|---|---|
| Counterion | Solvent | Procedure/Comments | PXRD Result | (vacuum dried) |
| HCl | Acetone | 40 mg S-5-MBPB (as freebase oil) + 5 vols. | S-5-MBPB | S-5-MBPB |
| | | solvent + 1.1:1 molar ratio counterion, stirred | Pattern 1B | Pattern 1A |
| | | at 40° C., clear solution after~3 hours, opened | | |
| | | for evaporation, off-white solids after | | |
| | | overnight evaporation, analyzed by PXRD, | | |
| | | placed in the vacuum oven at 40° C., analyzed | | |
| | | by PXRD after overnight drying | | |
| HBr | Acetone | 40 mg S-5-MBPB (as freebase oil) + 5 vols. | S-5-MBPB | S-5-MBPB |
| | | solvent + 1.1:1 molar ratio counterion, stirred | Pattern 2A | Pattern 2A + |
| | | at 40° C., clear solution after~3 hours, opened | with additional | 2B |
| | | for evaporation, yellow glassy solids after | peaks at 5.9°, | |
| | | overnight evaporation, analyzed by PXRD, | 26.7°, 29.2°, | |
| | | placed in the vacuum oven at 40° C. | 35.1° | |
| H₃PO₄ | Acetone | 40 mg S-5-MBPB (as freebase oil) + 5 vols. | S-5-MBPB | S-5-MBPB |
| | | solvent + 1.1:1 molar ratio counterion, stirred | Pattern 3A | Pattern 3A |
| | | at 40° C., white slurry after~3 hours, white | | |
| | | slurry after overnight stirring, centrifuged to | | |
| | | recover solids, analyzed by PXRD, placed in | | |
| | | the vacuum oven at 40° C., analyzed by | | |
| | | PXRD/DSC/TGA | | |
| HNO₃ | Acetone | 40 mg S-5-MBPB (as freebase oil) + 5 vols. | N/A | N/A |
| | | solvent + 1.1:1 molar ratio counterion, stirred | | |
| | | at 40° C., clear yellow solution after~3 hours, | | |
| | | opened for evaporation, orange gel after | | |
| | | overnight evaporation, added 5 vols. 1,4- | | |
| | | Dioxane, stirred at 40° C., orange clear solution | | |
| | | after~5 hours stirring, opened for evaporation, | | |
| | | orange-brown gel after overnight stirring, | | |
| | | terminated | | |
| Tartaric | Acetone | 40 mg S-5-MBPB (as freebase oil) + 5 vols. | N/A | N/A |
| | | solvent + 1.1:1 molar ratio counterion (stock | | |
| | | soln. in water), stirred at 40° C., clear solution | | |
| | | after~3 hours, opened for evaporation, clear | | |
| | | solution after overnight evaporation, beige gel | | |
| | | after 2 days evaporating, added 5 vols. 1,4- | | |
| | | Dioxane, stirred at 40° C., cloudy solution | | |
| | | after~3 hours stirring, opened for evaporation, | | |
| | | beige gel after overnight evaporation, | | |
| | | terminated | | |
| Succinic | Acetone | 40 mg S-5-MBPB (as freebase oil) + 5 vols. | Pattern 6A | Pattern 6A |
| | | solvent + 1.1:1 molar ratio counterion (stock | | |
| | | soln. in water), stirred at 40° C., clear solution | | |
| | | after~3 hours, opened for evaporation, clear | | |
| | | solution after overnight evaporation, beige gel | | |
| | | after 2 days evaporating, added 5 vols. 1,4- | | |
| | | Dioxane, stirred at 40° C., clear solution | | |
| | | after~3 hours stirring, opened for evaporation, | | |
| | | off-white solids after overnight evaporation, | | |
| | | analyzed by PXRD, placed in the vacuum oven | | |
| | | at 40° C., analyzed by PXRD/DSC | | |
| Oxalic | Acetone | 40 mg S-5-MBPB (as freebase oil) + 5 vols. | S-5-MBPB | S-5-MBPB |
| | | solvent + 1.1:1 molar ratio counterion (stock | Pattern 7A | Pattern 7A + |
| | | soln. in water), stirred at 40° C., clear solution | | another |
| | | after~3 hours, opened for evaporation, beige | | crystalline |
| | | solids after 2 days of evaporation, analyzed by | | phase |
| | | PXRD, placed in the vacuum oven at 40° C., | | |
| | | analyzed by PXRD | | |
| Maleic | Acetone | 40 mg S-5-MBPB (as freebase oil) + 5 vols. | S-5-MBPB | S-5-MBPB |
| | | solvent + 1.1:1 molar ratio counterion (stock | Pattern 8A | Pattern 8A |
| | | soln. in water), stirred at 40° C., clear solution | | |
| | | after~3 hours, opened for evaporation, beige | | |
| | | solids after 2 days of evaporation, analyzed by | | |
| | | PXRD, placed in the vacuum oven at 40° C., | | |
| | | analyzed by PXRD/DSC/TGA | | |
| Citric | Acetone | 40 mg S-5-MBPB (as freebase oil) + 5 vols. | S-5-MBPB | N/A |
| | | solvent + 1.1:1 molar ratio counterion (stock | Pattern 9A | |
| | | soln. in water), stirred at 40° C., clear solution | | |
| | | after~3 hours, opened for evaporation, beige | | |
| | | gel after 3 days of evaporation, added 5 vols. | | |
| | | 1,4-Dioxane, stirred at 40° C., beige clear | | |
| | | solution after~1 hour, opened for evaporation, | | |
| | | dry white powder-like solids after overnight | | |
| | | evaporation, analyzed by PXRD, placed in the | | |
| | | vacuum oven at 40° C. | | |
| Fumaric | Acetone | 40 mg S-5-MBPB (as freebase oil) + 5 vols. | S-5-MBPB | S-5-MBPB |
| | | solvent + 1.1:1 molar ratio counterion (stock | Pattern 10A | Pattern 10A |
| | | soln. in MeOH), stirred at 40° C., clear solution | | |
| | | after~3 hours, opened for evaporation, gel | | |
| | | after overnight evaporation, added 5 vols. 1,4- | | |
| | | Dioxane, stirred at 40° C., thick white pasty | | |
| | | solids after~5 hours stirring, added 10 vols. | | |
| | | 1,4-Dioxane to reslurry, stirred at 40° C. , thick | | |
| | | white slurry after overnight stirring, added 2.5 | | |
| | | vols 1,4-Dioxane to thin slurry, centrifuged to | | |
| | | recover solids, analyzed by PXRD, placed in | | |
| | | the vacuum oven at 40° C., analyzed by | | |
| | | PXRD/DSC/TGA | | |

### EXAMPLE 48 Salt Study Experiments of S-5-MBPB Pure Enantiomer in MeOH:Water

Salt studies of S-5-MBPB were conducted using the conditions in Table 44. The below technique was used to generate the XRPD patterns in FIG. 147, FIG. 148, FIG. 149, FIG. 151, FIG. 152, and FIG. 154 (For 40 mg of S-5-MBPB, 1 vol. solvent=40 μL).

TABLE 44
Salt screening experiments of S-5-MBPB

| | | | | PXRD Result |
|---|---|---|---|---|
| Counterion | Solvent | Procedure/Comments | PXRD Result | (vacuum dried) |
| HCl | MeOH:water | 40 mg S-5-MBPB (as freebase oil) + 5 vols. | S-5-MBPB | N/A |
| | 9:1 | solvent + 1.1:1 molar ratio counterion, stirred at | Pattern 1A + | |
| | | 40° C., clear solution after~3 hours, opened for | 1B | |
| | | evaporation, off-white solids after overnight | | |
| | | evaporation, analyzed by PXRD, placed in the | | |
| | | vacuum oven at 40° C. | | |
| HBr | MeOH:water | 40 mg S-5-MBPB (as freebase oil) + 5 vols. | S-5-MBPB | N/A |
| | 9:1 | solvent + 1.1:1 molar ratio counterion, stirred at | Pattern 2A | |
| | | 40° C., clear solution after~3 hours, opened for | with minor | |
| | | evaporation, yellow glassy solids after overnight | shoulders at | |
| | | evaporation, analyzed by PXRD, placed in the | 17.3° and | |
| | | vacuum oven at 40° C. | 23.1° | |
| H₃PO₄ | MeOH:water | 40 mg S-5-MBPB (as freebase oil) + 5 vols. | S-5-MBPB | S-5-MBPB |
| | 9:1 | solvent + 1.1:1 molar ratio counterion (stock soln. | Pattern 3A | Pattern 3A |
| | | in water), stirred at 40° C., clear solution after~3 | | |
| | | hours, opened for evaporation, gel after overnight | | |
| | | evaporation, added 5 vols. 1,4-Dioxane, stirred at | | |
| | | 40° C., thin white slurry after~5 hours, off-white | | |
| | | solids sitting in solvent after overnight stirring, | | |
| | | solvent pipetted out, analyzed by PXRD, placed in | | |
| | | the vacuum oven at 40° C., analyzed by PXRD | | |
| HNO₃ | MeOH:water | 40 mg S-5-MBPB (as freebase oil) + 5 vols. | N/A | N/A |
| | 9:1 | solvent + 1.1:1 molar ratio counterion, stirred at | | |
| | | 40° C., clear yellow solution after~3 hours, | | |
| | | opened for evaporation, orange gel after overnight | | |
| | | evaporation, added 5 vols. 1,4-Dioxane, stirred at | | |
| | | 40° C., orange clear solutions after~5 hours | | |
| | | stirring, opened for evaporation, dark gel after | | |
| | | overnight evaporation, terminated | | |
| Tartaric | MeOH:water | 40 mg S-5-MBPB (as freebase oil) + 5 vols. | N/A | N/A |
| | 9:1 | solvent + 1.1:1 molar ratio counterion (stock soln. | | |
| | | in water), stirred at 40° C., clear solution after~3 | | |
| | | hours, opened for evaporation, beige gel after 2 | | |
| | | days of evaporation, added 5 vols. chloroform, | | |
| | | stirred at 40° C., beige gel sitting in solvent | | |
| | | after~3 hours, opened for evaporation, beige gel | | |
| | | after overnight evaporation, terminated | | |
| Succinic | MeOH:water | 40 mg S-5-MBPB (as freebase oil) + 5 vols. | N/A | N/A |
| | 9:1 | solvent + 1.1:1 molar ratio counterion (stock soln. | | |
| | | in water), stirred at 40° C., clear solution after~3 | | |
| | | hours, opened for evaporation, clear solution after | | |
| | | overnight evaporation, beige gel after 2 days | | |
| | | evaporating, added 5 vols. 1,4-Dioxane, stirred at | | |
| | | 40° C., clear solution after~3 hours stirring, | | |
| | | opened for evaporation, beige gel after overnight | | |
| | | evaporation, terminated | | |
| Oxalic | MeOH:water | 40 mg S-5-MBPB (as freebase oil) + 5 vols. | S-5-MBPB | S-5-MBPB |
| | 9:1 | solvent + 1.1:1 molar ratio counterion (stock soln. | Pattern 7A | Pattern 7A + |
| | | in water), stirred at 40° C., clear solution after~3 | | another |
| | | hours, opened for evaporation, beige solids after 2 | | crystalline |
| | | days of evaporation, analyzed by PXRD, placed in | | phase |
| | | the vacuum oven at 40° C., analyzed by PXRD | | |
| Maleic | MeOH:water | 40 mg S-5-MBPB (as freebase oil) + 5 vols. | S-5-MBPB | S-5-MBPB |
| | 9:1 | solvent + 1.1:1 molar ratio counterion (stock soln. | Pattern 8A | Pattern 8A |
| | | in water), stirred at 40° C., clear solution after~3 | | |
| | | hours, opened for evaporation, clear solution after | | |
| | | overnight evaporation, off-white solids after 2 | | |
| | | days evaporating, analyzed by PXRD, placed in | | |
| | | vacuum oven at 40° C., analyzed by PXRD | | |
| Citric | MeOH:water | 40 mg S-5-MBPB (as freebase oil) + 5 vols. | N/A | N/A |
| | 9:1 | solvent + 1.1:1 molar ratio counterion (stock soln. | | |
| | | in water), stirred at 40° C., clear solution after~3 | | |
| | | hours, opened for evaporation, off-white clear | | |
| | | solution after 2 days of evaporation, beige gel after | | |
| | | 3 days evaporating, added 5 vols. chloroform, | | |
| | | stirred at 40° C., beige gel unsuspended in solvent | | |
| | | after overnight stirring, opened for evaporation, | | |
| | | terminated | | |
| Fumaric | MeOH:water | 40 mg S-5-MBPB (as freebase oil) + 5 vols. | Lower cryst. | N/A |
| | 9:1 | solvent + 1.1:1 molar ratio counterion (stock soln. | Pattern 10A | |
| | | in MeOH), stirred at 40° C., clear solution after~3 | with minor | |
| | | hours, opened for evaporation, gel after overnight | new peak at | |
| | | evaporation, added 5 vols. chloroform, stirred at | 19.2° | |
| | | 40° C., oil-phase separation after overnight stirring, | | |
| | | opened for evaporation, pasty beige solids after | | |
| | | overnight evaporation, analyzed by PXRD | | |

### EXAMPLE 49 Salt Study Experiments of S-5-MBPB Pure Enantiomer in ACN

Salt studies of S-5-MBPB were conducted using the conditions in Table 45. The below technique was used to generate the XRPD patterns in FIG. 141, FIG. 145, FIG. 147, FIG. 148, FIG. 149, FIG. 151, FIG. 152, FIG. 153, and FIG. 154 (For 40 mg of S-5-BPB, 1 vol. solvent=a).

TABLE 45
Salt screening experiments of S-5-MBPB

| | | | | PXRD Result |
|---|---|---|---|---|
| Counterion | Solvent | Procedure/Comments | PXRD Result | (vacuum dried) |
| HCl | ACN | 40 mg S-5-MBPB (as freebase oil) + 5 vols. | S-5-MBPB | Pattern 1A + |
| | | solvent + 1.1:1 molar ratio counterion, stirred at | Pattern 1A + | S-5-MBPB 1B |
| | | 40° C., clear solution after~3 hours, opened for | 1B | |
| | | evaporation, off-white solids after overnight | | |
| | | evaporation, analyzed by PXRD, placed in the | | |
| | | vacuum oven at 40° C., analyzed by PXRD | | |
| HBr | ACN | 40 mg S-5-MBPB (as freebase oil) + 5 vols. | S-5-MBPB | S-5-MBPB |
| | | solvent + 1.1:1 molar ratio counterion, stirred at | Pattern 2A | Pattern 2B |
| | | 40° C., clear solution after~3 hours, opened for | | |
| | | evaporation, yellow glassy solids after overnight | | |
| | | evaporation, analyzed by PXRD, placed in the | | |
| | | vacuum oven at 40° C., analyzed by | | |
| | | PXRD/DSC/TGA | | |
| H₃PO₄ | ACN | 40 mg S-5-MBPB (as freebase oil) + 5 vols. | S-5-MBPB | S-5-MBPB |
| | | solvent + 1.1:1 molar ratio counterion, stirred at | Pattern 3A | Pattern 3A |
| | | 40° C., wet off-white solids sitting in solvent | | |
| | | after~3 hours, wet off-white solids sitting in solvent | | |
| | | after overnight stirring, opened for evaporation, | | |
| | | off-white wet solids after overnight evaporation, | | |
| | | analyzed by PXRD, placed in vacuum oven at 40° | | |
| | | C., analyzed by PXRD | | |
| HNO₃ | ACN | 40 mg S-5-MBPB (as freebase oil) + 5 vols. | N/A | N/A |
| | | solvent + 1.1:1 molar ratio counterion, stirred at | | |
| | | 40° C., clear yellow solution after~3 hours, | | |
| | | opened for evaporation, orange gel after overnight | | |
| | | evaporation, added 5 vols. 1,4-Dioxane, stirred at | | |
| | | 40° C., orange clear solutions after~5 hours | | |
| | | stirring, orange-brown gel after overnight | | |
| | | evaporation, terminated | | |
| Tartaric | ACN | 40 mg S-5-MBPB (as freebase oil) + 5 vols. | N/A | N/A |
| | | solvent + 1.1:1 molar ratio counterion (stock soln. | | |
| | | in water), stirred at 40° C., clear solution after~3 | | |
| | | hours, opened for evaporation, clear solution after | | |
| | | overnight evaporation, beige gel after 2 days | | |
| | | evaporating, added 5 vols. DCM, stirred at 40° C., | | |
| | | beige gel unsuspended in solvent after~3 hours | | |
| | | stirring, opened for evaporation, beige gel after | | |
| | | overnight evaporation, terminated | | |
| Succinic | ACN | 40 mg S-5-MBPB (as freebase oil) + 5 vols. | N/A | N/A |
| | | solvent + 1.1:1 molar ratio counterion (stock soln. | | |
| | | in water), stirred at 40° C., clear solution after~3 | | |
| | | hours, opened for evaporation, clear solution after | | |
| | | overnight evaporation, orange gel after 2 days | | |
| | | evaporating, added 5 vols. DCM, stirred at 40° C., | | |
| | | clear solution after~3 hours stirring, opened for | | |
| | | evaporation, beige gel after overnight evaporation, | | |
| | | terminated | | |
| Oxalic | ACN | 40 mg S-5-MBPB (as freebase oil) + 5 vols. | S-5-MBPB | S-5-MBPB |
| | | solvent + 1.1:1 molar ratio counterion (stock soln. | Pattern 7A + | Pattern 7A + |
| | | in water), stirred at 40° C., clear solution after~3 | another | another |
| | | hours, opened for evaporation, clear solution after | crystalline | crystalline |
| | | overnight evaporation, off-white solids after 2 | phase | phase |
| | | days evaporating, analyzed by PXRD, placed in | | |
| | | vacuum oven at 40° C., analyzed by PXRD | | |
| Maleic | ACN | 40 mg S-5-MBPB (as freebase oil) + 5 vols. | S-5-MBPB | S-5-MBPB |
| | | solvent + 1.1:1 molar ratio counterion (stock soln. | Pattern 8A | Pattern 8A |
| | | in water), stirred at 40° C., clear solution after~3 | | |
| | | hours, opened for evaporation, clear solution after | | |
| | | overnight evaporation, off-white solids after 2 | | |
| | | days evaporating, analyzed by PXRD, placed in | | |
| | | vacuum oven at 40° C., analyzed by PXRD | | |
| Citric | ACN | 40 mg S-5-MBPB (as freebase oil) + 5 vols. | S-5-MBPB | S-5-MBPB |
| | | solvent + 1.1:1 molar ratio counterion (stock soln. | Pattern 9A | Pattern 9A |
| | | in water), stirred at 40° C., clear solution after~3 | | |
| | | hours, opened for evaporation, beige clear solution | | |
| | | after 2 days of evaporation, beige gel after 3 days | | |
| | | evaporating, added 5 vols. DCM to reslurry, | | |
| | | stirred at 40° C., wet white solids after overnight | | |
| | | stirring, opened to allow solids to dry, analyzed by | | |
| | | PXRD, placed in vacuum oven at 40° C., analyzed | | |
| | | by PXRD, analyzed by DSC | | |
| Fumaric | ACN | 40 mg S-5-MBPB (as freebase oil) + 5 vols. | Lower cryst. | N/A |
| | | solvent + 1.1:1 molar ratio counterion (stock soln. | Pattern 10A | |
| | | in MeOH), stirred at 40° C., clear solution after~3 | with minor | |
| | | hours, opened for evaporation, gel after overnight | new peak at | |
| | | evaporation, added 5 vols. chloroform, stirred at | 19.3° | |
| | | 40° C., oil-phase separation after overnight stirring, | (similar to | |
| | | opened for evaporation, pasty beige solids after | NB1018A- | |
| | | overnight evaporation, analyzed by PXRD | 117-20) | |

### EXAMPLE 50. Scale Up of R-5-MBPB Liquid-Liquid Extraction

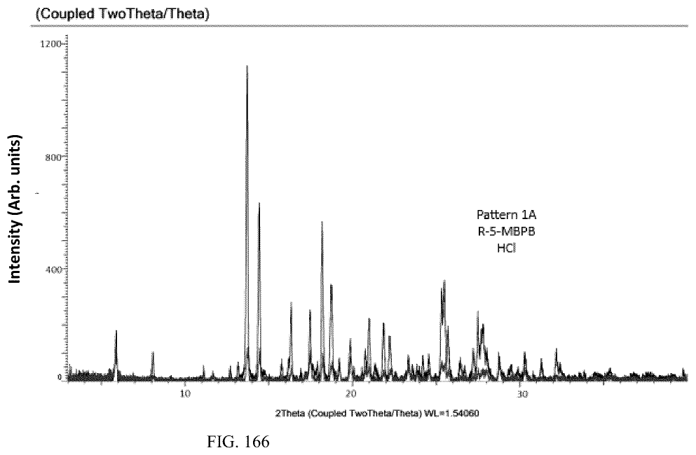

Scale ups of the R-5-MBPB liquid-liquid extraction expt. were performed to isolate large amounts of the Freebase API from the R-5-MBPB HCl salt. ˜0.5 g API (as HCl salt) plus 36.7 vols. (18.4 mL) of EtOAc were added to NaOH soln. in water (5 mg/mL) for 1.1:1 base:salt molar ratio. Organic phase was then recovered, the solvent was allowed to evaporate at 40° C., and the resulting brown gel was analyzed by NMR. Table 46 describes scale up conditions of liquid-liquid extraction conditions of R-5-MBPB. Pattern 1A (FIG. 166) shows XRPD diffractogram of R-5-MBPB HCl.

TABLE 46
Liquid-liquid extraction scale up experiments of R-5-MBPB

| | Solvent | |
|---|---|---|
| Base | (Density) | Procedure/Comments |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | The top organic layers of R-5-MBPB were pipetted out and combined, |
| | (0.902 g/mL) | concentration estimated as 23.1 mg/mL |

### EXAMPLE 51 Salt Study Experiments of R-5-MBPB Pure Enantiomer in Acetone

Salt studies of R-5-1MBPB were conducted using the conditions in Table 47. The below technique was used to generate the XRPD patterns in FIG. 163, FIG. 164, FIG. 167, FIG. 168, and FIG. 169 (For 40 mg of R-5-MBPB, 1 vol. solvent=40 μL).

TABLE 47
Salt screening experiments of R-5-MBPB

| Counterion | Solvent | Procedure/Comments | PXRD Result |
|---|---|---|---|
| H₃PO₄ | Acetone | 40 mg R-5-MBPB (as freebase oil) + 5 vols. solvent + | R-5-MBPB |
| | | 1.1:1 molar ratio counterion, stirred at 40° C., thick white | Pattern 3A |
| | | slurry after~1 hour stirring, added 5 vols. solvent to thin | |
| | | slurry, stirred at 40° C., centrifuged to recover solids, | |
| | | analyzed by PXRD | |
| Maleic | Acetone | 40 mg R-5-MBPB (as freebase oil) + 5 vols. solvent + | R-5-MBPB |
| | | 1.1:1 molar ratio counterion (stock soln. in water), stirred | Pattern 8A |
| | | at 40° C., beige clear solution immediately after~1 hour | |
| | | stirring, opened for evaporation, beige clear solution after | |
| | | overnight evaporation, off-white solids after 3 days | |
| | | evaporating, analyzed by PXRD | |
| Fumaric | Acetone | 40 mg R-5-MBPB (as freebase oil) + 5 vols. solvent + | R-5-MBPB |
| | | 1.1:1 molar ratio counterion (stock soln. in MeOH), | Pattern 10A |
| | | stirred at 40° C., beige clear solution immediately after~1 | |
| | | hour stirring, opened for evaporation, beige gel after | |
| | | overnight evaporation, added 5 vols. 1,4-Dioxane to | |
| | | reslurry, stirred at 40° C., cloudy solution after~6.5 hours | |
| | | stirring, opened for evaporation, off-white solids after 3 | |
| | | days evaporating, analyzed by PXRD | |

### EXAMPLE 52 Salt Study Experiments of R-5-MBPB Pure Enantiomer in 1,4-Dioxane

Salt studies of R-5-1MBPB were conducted using the conditions in Table 48. The below technique was used to generate the XRPD patterns in FIG. 165 and FIG. 169 (For 40 mg of R-5-MBPB, 1 vol. solvent=40 μL).

TABLE 48
Salt screening experiments of R-5-MBPB

| Counterion | Solvent | Procedure/Comments | PXRD Result |
|---|---|---|---|
| Fumaric | 1,4-Dioxane | 40 mg R-5-MBPB (as freebase oil) + 10 vols. solvent + | R-5-MBPB |
| | | 1.1:1 molar ratio counterion (stock soln. in MeOH), | Pattern 10A |
| | | stirred at 40° C., beige clear solution immediately | |
| | | after~1 hour stirring, opened for evaporation, beige gel | |
| | | after overnight evaporation, added 5 vols. DCM to | |
| | | reslurry, stirred at 40° C., oiled out after~4 hours | |
| | | stirring, opened for evaporation, beige gel after~2.5 | |
| | | hours evaporating, added 5 vols. EtOAc to reslurry, | |
| | | stirred at 40° C., thick white slurry after~10 minutes | |
| | | stirring, added 9 vols. EtOAc to thin slurry, thick | |
| | | white slurry after 3 days stirring, added 10 vols. | |
| | | EtOAc to thin slurry, centrifuged to recover solids, | |
| | | analyzed by PXRD | |

### EXAMPLE 53. Scale Up of R-6-MBPB Liquid-Liquid Extraction

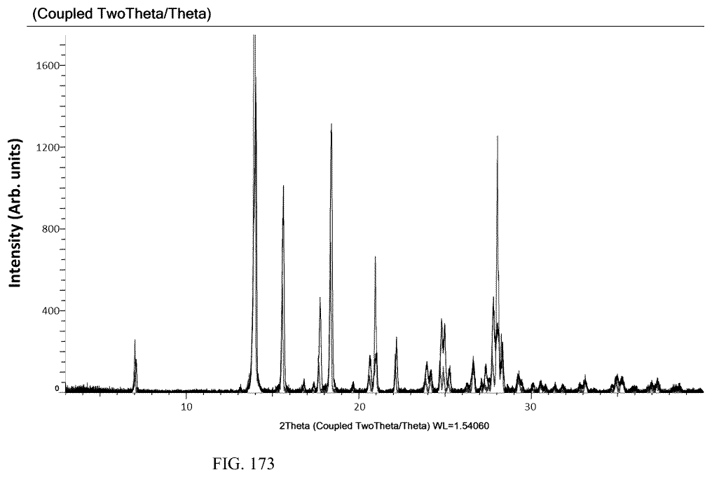

Scale ups of the R-6-1MBPB liquid-liquid extraction expt. were performed to isolate large amounts of the Freebase API from the R-6-MBPB HCl salt. ˜0.5 g API (as HCl salt) plus 36.7 vols. (18.4 mL) of EtOAc were added to NaOH soln. in water (5 mg/mL) for 1.1:1 base:salt molar ratio. Organic phase was then recovered, the solvent was allowed to evaporate at 40° C., and the resulting brown gel was analyzed by NMR. Table 49 describes scale up conditions of liquid-liquid extraction conditions of R-6-MBPB. Pattern 1A (FIG. 173) shows a comparison of XRPD diffractogram of R-6-MBPB (HCl salt).

TABLE 49
Liquid-liquid extraction scale up experiments of R-6-MBPB

| | Solvent | |
|---|---|---|
| Base | (Density) | Procedure/Comments |
| NaOH | EtOAc | 0.5 g API (as HCl salt) + 36.7 vols. solvent + base stock soln. in water (1.1:1 |
| | (0.902 g/mL) | molar ratio), vigorously shaken and allowed to separate |
| NaOH | EtOAc | 0.5 g API (as HCI salt) + 36.7 vols. solvent + base stock soln. in water (1.1:1 |
| | (0.902 g/mL) | molar ratio), vigorously shaken and allowed to separate |
| NaOH | EtOAc | The top organic layers of R-6-MBPB were pipetted out and combined, |
| | (0.902 g/mL) | concentration estimated as 23.1 mg/mL |

### EXAMPLE 54 Salt Study Experiments of R-6-MBPB Pure Enantiomer in ACN

Salt studies of R-6-MBPB were conducted using the conditions in Table 50. The below technique was used to generate the XRPD patterns in FIG. 170, FIG. 171, FIG. 172, FIG. 174, FIG. 175 and FIG. 65 (For 40 mg of R-6-MBPB, 1 vol. solvent=40 μL).

TABLE 50
Salt screening experiments of R-6-MBPB

| Counterion | Solvent | Procedure/Comments | PXRD Result |
|---|---|---|---|
| HCl | ACN | 40 mg R-6-MBPB (as freebase oil) + 5 vols. solvent + | Pattern 1A |
| | | 1.1:1 molar ratio counterion, stirred at 40° C., clear | |
| | | yellow solution after~1 hour, opened for evaporation, | |
| | | orange-brown glassy solids after overnight evaporation, | |
| | | analyzed by PXRD | |
| HBr | ACN | 40 mg R-6-MBPB (as freebase oil) + 5 vols. solvent + | Pattern 2A |
| | | 1.1:1 molar ratio counterion, stirred at 40° C., clear | |
| | | yellow solution after~1 hour, opened for evaporation, | |
| | | orange-brown glassy solids after overnight evaporation, | |
| | | analyzed by PXRD | |
| Oxalic | ACN | 40 mg R-6-MBPB (as freebase oil) + 5 vols. solvent + | Pattern 9A |
| | | 1.1:1 molar ratio counterion (stock soln. in water), stirred | |
| | | at 40° C., clear yellow solution after~1 hour, opened for | |
| | | evaporation, off-white powder-like solids after overnight | |
| | | evaporation, analyzed by PXRD | |

### EXAMPLE 55: In Vitro Binding Site Studies

RS-6-MBPB was tested for agonist and antagonist activity against 5-HT_{2A} and 5-HT₂B and the results are shown in Table 51. RS-6-MBPB, R-6-MBPB, and S-6-MBPB were assessed at 5-HT_{1B} with results shown in Table 52.

5-HT₂A and 5-HT₂B Agonist and Antagonist Assays

The DiscoveRx Calcium NWPLUS Assay was used for detection of changes in intracellular calcium as signaled by an increase of dye fluorescence in cells expressing 5-HT₂A receptors. Signal was measured on a fluorescent plate reader equipped with fluidic handling capable of detecting rapid changes in fluorescence upon compound stimulation.

To conduct the assay, cell lines were expanded from freezer stocks according to standard procedures. Cells (10,000cells/well) were seeded in a total volume of 50 L (200 cells/L) into black-walled, clear-bottom, Poly-D-lysine coated 384-well microplates and incubated at 37-C for the appropriate time prior to testing. DMSO concentration for all readouts was <0.2%.

Assays were performed in 1× DyeLoading Buffer consisting of 1× Dye (DiscoverX, Calcium No WashPLUS kit, Catalog No. 90-0091), 1× Additive A and 2.5 mM Probenecid in HBSS/20 mM Hepes. Probenecid was prepared fresh. Cells were loaded with dye prior to testing. Media was aspirated from cells and replaced with 25 μL Dye Loading Buffer. Test compounds were assayed at 10 concentrations with the highest concentration either 30 or 10 μM and subsequent concentrations using a 0.33 dilution factor. Cells with testing sample were incubated for 45 minutes at 37-C and then 20 minutes at room temperature. After dye loading, cells were removed from the incubator and 25 μL of 2× compound in HBSS/20 mM Hepes was added using a FLIPR Tetra (MDS). For 5-HT₂A assays, serotonin and altanserin were used as agonist and antagonist reference controls. For 5-HT₂B assays, these were serotonin and LY272015.

For antagonist determination, cells were pre-incubated with sample followed by agonist challenge at the EC₈₀ concentration. After dye loading, cells were removed from the incubator and L 2× sample was added. Cells were incubated for 30 minutes at room temperature in the dark to equilibrate plate temperature. After incubation, antagonist determination was initiated with addition of 25 μL 1× compound with 3× EC₈₀ agonist using FLIPR.

Compound agonist activity was measured on a FLIPR Tetra. Calcium mobilization was monitored for 2 minutes with a 5 second baseline read. FLIPR read-Area under the curve was calculated for the two minute read. Compound activity was analyzed using CBIS data analysis suite (ChemInnovation, CA). Percentage activity was calculated as 100%×(mean RFU of test sample−mean RFU of vehicle control)/(mean MAX RFU control ligand −mean RFU of vehicle control). For antagonist mode assays, percentage inhibition was calculated as 100%×(1−(mean RFU of test sample−mean RFU of vehicle control)/(mean RFU of EC₈₀ control−mean RFU of vehicle control)).

TABLE 51
Agonist and Antagonist activity against 5-HT2A and 5-HT2B

| | 5HT_{2A} | 5HT_{2A} | 5HT_{2B} | 5HT_{2B} |
|---|---|---|---|---|
| | Agonist EC50 | Antagonist | Agonist | Antagonist |
| Compound | (μM) | IC50 (μM) | EC50 (μM) | IC50 (μM) |
| 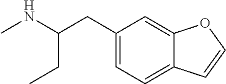 RS-6-MBPB | ND | ND | ND | 3.08 |

ND is none detectable, using concentrations up to 30 μM

### EXAMPLE 56: 5-HT1BR cAMP Secondary Messenger Agonist Assay

The 5-HT_{1B}R cAMP secondary messenger agonist assay used a panel of CHO-K1 cell lines stably expressing non-tagged GPCRs that endogenously signal through cAMP. Hit Hunter® cAMP assays monitored the activation of a GPCR via Gi and Gs secondary messenger signaling in a homogenous, non-imaging assay format using DiscoverX Enzyme Fragment Complementation (EFC) with β-galactosidase as the functional endpoint.

The enzyme was split into two complementary portions: Enzyme Acceptor (EA) and Enzyme Donor (ED). Exogenously introduced ED fused to cAMP (ED-cAMP) competed with endogenously generated cAMP for binding to an anti-cAMP-specific antibody. Active β-galactosidase was formed by complementation of exogenous EA to any unbound ED-cAMP. Active enzyme could then convert a chemiluminescent substrate, generating an output signal detectable on a standard microplate reader.

Cell lines were expanded from freezer stocks according to standard procedures. Cells were seeded in a total volume of 20 μL into white walled, 384-well microplates and incubated at 37-C for the appropriate time prior to testing. cAMP modulation was determined using the DiscoverX HitHunter cAMP XS+ assay.

For agonist determination, cells were incubated with sample (in the presence of EC₈₀ forskolin to induce response if measuring Gi secondary messenger signaling). Media was aspirated from cells and replaced with 15 μL 2:1 HBSS/10 mM Hepes: cAMP XS+Ab reagent. Intermediate dilution of sample stocks was performed to generate 4× sample in assay buffer (optionally containing 4× EC₈₀ forskolin). 5 μL of 4× sample was added to cells and incubated at 37° C. or room temperature for 30 or 60 minutes, as appropriate. Final assay vehicle concentration was 1%. The results are shown in Table 53.

Surprisingly, the benzofuran derivative of the current invention is 5-HT1BR agonist. Direct stimulation of 5-HT1BR has not been previously documented with drugs producing MDMA-like effects and MDMA itself does not bind to the 5-HT1BR (Ray. 2010. PloS one, 5(2), e9019). Indirect stimulation of 5-HT1BR, secondary to elevated extracellular serotonin, has been hypothesized to be required for the prosocial effects of MDMA (Heifets et al. 2019. Science translational medicine, 11(522)), while other aspects of entactogen effects have been attributed to monoamine release (e.g., Luethi & Liechti. 2020. Archives of toxicology, 94(4), 1085-1133).

Thus, in one embodiment, the unique ratios of 5-HT1BR stimulation and monoamine release displayed by 6-MBPB enable different profiles of therapeutic effects that cannot be achieved by MDMA or other known entactogens.

TABLE 52
5-HT1B Agonist Effects of 6-MBPB

| | 5-HT_{1B}R | |
|---|---|---|
| | EC50 | Hill |
| Compound | (μM) | Coef. |
| 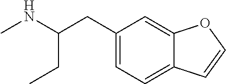 RS-6-MBPB | 1.95124 | 0.89 |
| 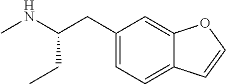 S-6-MBPB | 1.72234 | 1.1131 |
| 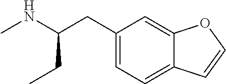 R-6-MBPB | 5.75045 | 0.8121 |

### EXAMPLE 57: Human Serotonin Transporter (SERT, SLC6A4) Functional Antagonist Uptake Assay

Benzofuran derivatives were evaluated for inhibiting the human 5-HT transporter (hSERT) as expressed in CHO cells using an antagonist radioligand assay (Tatsumi, M. et al. (1999), Eur. J. Pharmacol., 368: 277-283). Compound binding was calculated as a percent inhibition of the binding of 2 nM [³H]imipramine using a scintillation method and inhibition constants (Ki) were calculated using the Cheng Prusoff equation. Test compounds were assayed in three trials at 300, 94.868, 30, 9.4868, 0.3, and 0.94868 μM.

All tested compounds showed inhibition of hSERT at the tested concentrations. However, in two cases (the enantiomers of 5-MBPB), the lowest concentration of 0.94868 μM was too high to accurately estimate IC₅₀ values and Ki values. For S-(+)-5-MBPB the IC₅₀ appeared close to 0.094868 μM, while for R-(−)-5-MBPB the IC₅₀ appeared close to 0.94868 μM.

When compounds are substrates for monoamine transporters instead of solely inhibitors, it is known that IC₅₀ values underestimate their potency for interacting with these transporters (Ilic, M. et al. (2020), Frontiers in Pharmacology 11: 673).

TABLE 53
Human Serotonin Transporter Functional Antagonist Uptake Assay

| Compound | SERT IC50 (μM) | SERT Ki (μM) |
|---|---|---|
| 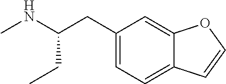 S-(+)-6-MBPB | 2.70 | 1.20 |
| 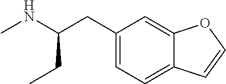 R-(−)-6-MBPB | 5.70 | 2.60 |

### EXAMPLE 58: Effects of Substituted Benzofurans on Extracellular Monoamines

Select compounds of the present invention were studied for their effect on extracellular serotonin and compared to MDMA. The results are shown in Table 54.

TABLE 54
Effects of Substituted Benzofurans on Extracellular Serotonin

| | Inhibition of | |
|---|---|---|
| | [3H]5-HT | [3H]5-HT |
| | Uptake at | release via |
| | SERT | SERT |
| Compound | IC₅₀ (nM) | EC₅₀ (nM) |
| 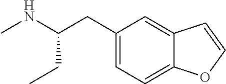 S-6-MBPB | 216.8 ± 45.8 | 54.1 ± 33.9 |
| 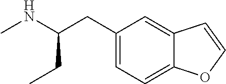 R-6-MBPB | 702.5 ± 261.7 | 171.7 ± 82.2 |
| 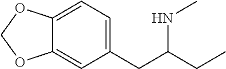 MDMA | 384.5 ± 32.8 | 94.3 ± 13.6 |

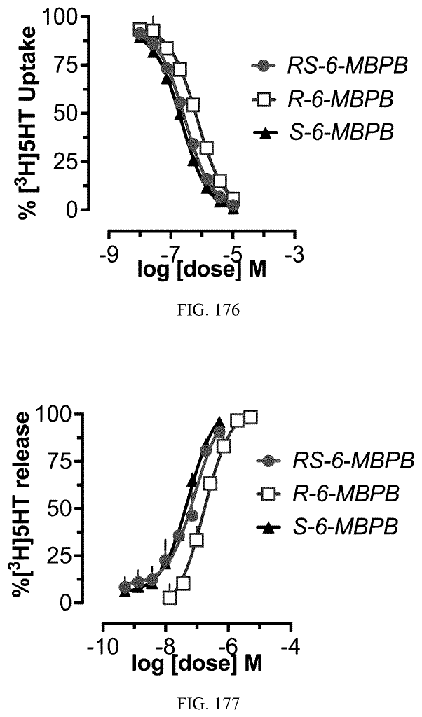

The compounds were efficacious at rapidly increasing extracellular serotonin, which produces rapid therapeutic effects. FIGS. 176, 177, and 178 show in vitro rat synaptosome assay results that demonstrate serotonin reuptake inhibition and release of serotonin, dopamine and norepinephrine.

Male Sprague-Dawley rats (Charles River, Kingston, NY, USA) were used for the synaptosome assays. Rats were group-housed with free access to food and water, under a 12 hour light/dark cycle with lights on at 0700 hours. Rats were euthanized by CO₂ narcosis, and synaptosomes were prepared from brains using standard procedures (Rothman, R. B., & Baumann, M. H. (2003) Monoamine transporters and psychostimulant drugs. European journal of pharmacology, 479(1-3), 23-40) Transporter uptake and release assays were performed as described previously (Solis et al. (2017). N-Alkylated analogs of 4-methylamphetamine (4-MA) differentially affect monoamine transporters and abuse liability. Neuropsychopharmacology, 42(10), 1950-1961). In brief, synaptosomes were prepared from whole brain minus caudate and cerebellum for serotonin (5-HT) transporter (SERT) assays.

For the SERT uptake inhibition assay, 5 nM [3H]5-HT was used. To optimize uptake for a single transporter, unlabeled blockers were included to prevent the uptake of [3H]5-HT by competing transporters. Uptake inhibition was initiated by incubating synaptosomes with various doses of test compound and [3H]5-HT in Krebs-phosphate buffer. Uptake assay was terminated by rapid vacuum filtration and retained radioactivity was quantified with liquid scintillation counting (Baumann et al. (2013) Powerful cocaine-like actions of 3, 4-methylenedioxypyrovalerone (MDPV), a principal constituent of psychoactive ‘bath salts’ products. Neuropsychopharmacology, 38(4), 552-562). Results of the experiment are shown in FIG. 176 (for RS-6-MBPB, R-6-MBPB, and S-6-MBPB).

For the release assay, 5 nM [3H]5-HT was used for SERT. All buffers used in the release assay contained 1 μM reserpine to block vesicular uptake of substrates. The selectivity of the release assay was optimized for a single transporter by including unlabeled blockers to prevent the uptake of [3H]5-HT by competing transporters. Synaptosomes were preloaded with radiolabeled substrate in Krebs-phosphate buffer for 1 hour to reach steady state. The release assay was initiated by incubating preloaded synaptosomes with various concentrations of the test drug. Release was terminated by vacuum filtration and retained radioactivity quantified by liquid scintillation counting. Results of the experiment are shown in FIG. 177 (for RS-6-MBPB, R-6-MBPB, and S-6-MBPB).

Effects of test drugs on release were expressed as a percent of maximal release, with maximal release (i.e., 100% Eₘₐₓ) defined as the release produced by tyramine at doses that evoked the efflux of all ‘releasable’ tritium by synaptosomes (100 μM tyramine for SERT assay conditions). Effects of test drugs on uptake inhibition and release were analyzed by nonlinear regression. Dose-response values for the uptake inhibition and release were fit to the equation, Y(x)=Yₘᵢₙ+(Yₘₐₓ−Yₘᵢₙ)/(1+10exp[(log P₅₀−log x)]×n), where x was the concentration of the compound tested, Y(x) was the response measured, Yₘₐₓ was the maximal response, P₅₀ was either IC₅₀ (the concentration that yielded half-maximal uptake inhibition response) or EC₅₀ (the concentration that yielded half-maximal release), and n was the Hill slope parameter.

Similarly, caudate tissue can be used for dopamine transporter (DAT) and whole brain minus caudate and cerebellum used for norepinephrine transporter (NET) assays. For other uptake inhibition assays, 5 nM [3H]dopamine or [3H]norepinephrine can be used for DAT or NET assays respectively. To optimize uptake for a single transporter, unlabeled blockers are included to prevent the uptake of [3H]transmitter by competing transporters. Uptake inhibition is initiated by incubating synaptosomes with various doses of test compound and [3H]transmitter in Krebs-phosphate buffer. Uptake assays are terminated by rapid vacuum filtration and retained radioactivity is quantified with liquid scintillation counting (Baumann et al. (2013). Powerful cocaine-like actions of 3, 4-methylenedioxypyrovalerone (MDPV), a principal constituent of psychoactive ‘bath salts’ products. Neuropsychopharmacology, 38(4), 552-562).

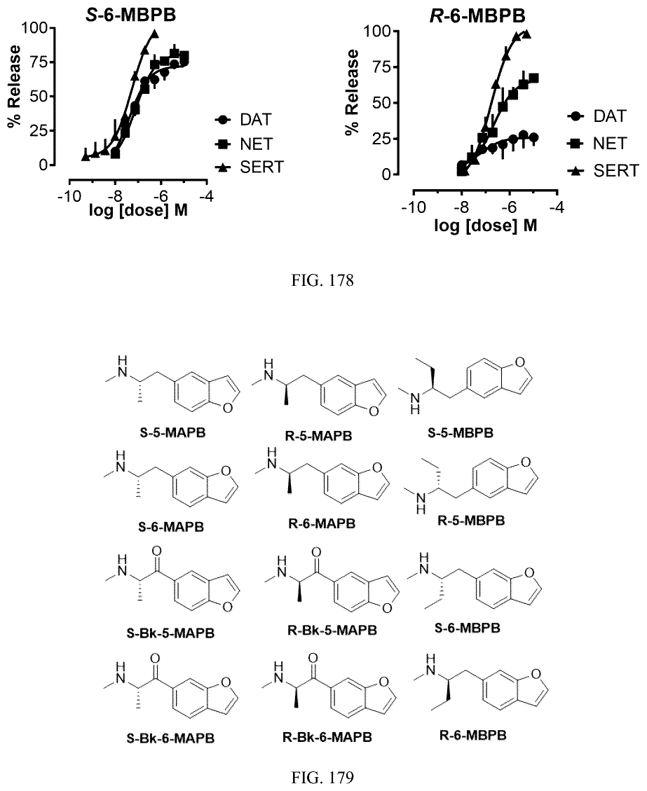

For the present results, 9 nM [3H]MPP+ was used as the radiolabeled substrate for DAT and NET. All buffers in the release assay contain 1 μM reserpine to block vesicular uptake of substrates. The selectivity of release assays was optimized for a single transporter by including unlabeled blockers to prevent the uptake of [3H]MPP+ or [3H]5-HT by competing transporters. Synaptosomes were preloaded with radiolabeled substrate in Krebs-phosphate buffer for 1 h to reach steady state. Release assays were initiated by incubating preloaded synaptosomes with various concentrations of the test drug. Release was terminated by vacuum filtration and retained radioactivity quantified by liquid scintillation counting. Results are shown in FIG. 178.

### EXAMPLE 59: Evaluation of Side Effects of Entactogens

Adverse effects of an entactogen include formation of tolerance to entactogens, headache, difficulty concentrating, lack of appetite, lack of energy, and decreased mood. In addition to these mild toxicities, MDMA is associated with a number of more severe toxicities, including but not limited to acute and chronic cardiovascular changes, hepatotoxicity, hyperthermic syndromes, hyponatremia, and neurotoxicity (see the MDMA Investigator's Brochure, 14th Edition: Mar. 18, 2022, and references therein, available from the sponsor of MDMA clinical trials at MAPS.org).

Acute physiological changes can be measured in humans with standard clinical methods (blood pressure cuffs, 3-lead EKG, tympanic or oral temperature, serum sodium, etc.), with measures usually collected before and at scheduled intervals after an entactogen. For example, measures may be collected before, 0.5, 0.75, 1, 1.5, 2, 2.5, 3, 4, 5, 6, and 8 hours after an entactogen. Maximum change from baseline and area-under-the-effects-versus-time-curve may be used as summary measures and statistically compared to a placebo control condition.

To measure adverse symptoms, patients can be asked to complete a self-report symptom questionnaire, such as the Subjective Drug Effects Questionnaire (SDEQ) or List of Complaints. The SDEQ is a 272-item self-report instrument measuring perceptual, mood, and somatic changes caused by drugs including hallucinogens like LSD (Katz et al. 1968. J Abnorm Psychology 73:1-14). It has also been used to measure the therapeutic and adverse effects of MDMA (Harris et al. 2002. Psychopharmacology, 162(4), 396-405). The List of Complaints is a 66-item questionnaire that measures physical and general discomfort and is sensitive to entactogen-related complaints (e.g., Vizeli & Liechti. 2017. Journal of Psychopharmacology, 31(5), 576-588).

Alternatively, individual items can be taken from the SDEQ or List of Complaints in order to create more focused questionnaires and reduce the burden of filling out time-consuming paperwork on participants. To measure tolerance formation, a global measure of the intensity of therapeutic effects can be used, such as the question “on a scale from 0 to 100 where 0 is no ‘good drug effect’ and 100 is the most ‘good drug effect’ you have ever felt, how would you rate this drug experience?”

In some embodiments, the questionnaire will be administered approximately 7 hours after a patient takes MDMA or another entactogen (with instructions to answer for the time since taking the entactogen) and then daily (with instructions to answer for the last 24 hours) for up to 96 hours after the entactogen was taken. Decreases in adverse effects of a compound compared to MDMA can be shown by comparing the intensity (for the tolerance question) or prevalence (for other symptom questions) of effects that occur. Prevalence of adverse effects including formation of tolerance to entactogens, headache, difficulty concentrating, lack of appetite, lack of energy, and decreased mood may be decreased by approximately 10%, 20%, 30%, 40%, 50%, 60%, 70%, 80%, 90%, 99%, or 100%.

As an alternative to measuring side effects of entactogens in clinical trials, preclinical studies in rodents may also be used. Appropriate tasks and behaviors that may be used to measure side effects include physiological measures (heart rate, blood pressure, body temperature), the modified Irwin procedure or functional observational battery (Irwin, Psychopharmacologia, 13, 222-257, 1968), and locomotor activity (such as distance traveled, rearing frequency, and rearing duration; Piper et la., J Pharmacol Exp Ther, 317, 838-849, 2006). In these studies, an entactogen is administered at different doses (including a vehicle only placebo) to different groups of animals and measures are made at scheduled times before and after administration. For example, 0, 1.5, 3, 15, and 30 mg/kg of a compound may be administered intraperitoneally, and measures made before and 15, 30, 60, 120 and 180 minutes and 12, 24, 36, and 48 hours after administration of the test substance.

### EXAMPLE 60: Neuroplasticity Assay in Primary Cortical Neurons

Compounds of the current invention can be considered psychoplastogens, that is, small molecules that are able to induce rapid neuroplasticity (Olson, 2018, Journal of experimental neuroscience, 12, 1179069518800508). One exemplary method for measuring this, a neurite outgrowth assay conducted in murine primary cortical neurons, is provided below. Other methods are well known in the literature (e.g. Olson, 2018, Journal of experimental neuroscience, 12, 1179069518800508; Ly et al. Cell reports 23, no. 11 (2018): 3170-3182; and references therein).

Primary cortical neurons are prepared from timed pregnant wild-type C57BL/6JRccHsd mice at E18. Animals are sacrificed (see section 3.3.1) and embryos are dissected in Calcium and Magnesium free Hanks Balanced Salt Solution (CMF-HBSS) containing 15 mM HEPES and 10 mM NaHCO₃, pH 7.2. Embryos are decapitated, skin and skull gently removed and hemispheres are separated. After removing meninges and brain stem, the hippocampi are isolated, chopped with a sterile razor blade in Chop solution (Hibernate-E without Calcium containing 2% B-27) and digested in 2 mg/mL papain (Worthington) dissolved in Hibernate-E without Calcium for 30 minutes (±5 min) at 30° C. Hippocampi are triturated for 10-15 times with a fire-polished silanized Pasteur pipette in Hibernate-E without Calcium containing 2% B-27, 0.01% DNaseI, 1 mg/mL BSA, and 1 mg/mL Ovomucoid Inhibitor. Undispersed pieces are allowed to settle by gravity for 1 min and the supernatant is centrifuged for 3 min at 228 g. The pellet is resuspended in Hibernate-E containing 2% B-27, 0.01% DNaseI, 1 mg/ml BSA, 1 mg/mL Ovomucoid Inhibitor and diluted with Hibernate-E containing 2% B-27. After the second centrifugation step (3 min at 228 g), the pellet is resuspended in nutrition medium (Neurobasal, 2% B-27, 0.5 mM glutamine, 1% Penicillin-Streptomycin).

Cells are counted in a hemacytometer and seeded in nutrition medium on poly-D-lysine pre-coated 96-well plates at a density of 2.6×104 cells/well. Cells are cultured at 37° C.; 95% humidity and 5% CO2. All wells are handled the same way.

The experiment is performed in adequate technical replicates for all groups, for example five replicates.

On the day of preparation (DIV1), mouse cortical neurons are seeded on poly-D-lysine pre-coated 96-well plates at a density of 2.6×104 cells per well.

On DIV2, cells are treated with test compounds at concentrations selected based on their EC50 at SERT release or 5-HT receptor agonism for three different time points (4 h, 8 h and 24 h), followed by a complete medium change. Additionally, cells are treated with 40 ng/mL of a positive control (Fibroblast growth factor, FGF) or vehicle control (VC) for 48 h.

The experiment is carried out with several, for example five, technical replicates per condition, vehicle treated cells serve as control.

Treated primary neurons are fixed on DIV4 by addition of equal volume 4% paraformaldehyde (PFA) to the medium at room temperature (RT) for 30 minutes.

Cells are rinsed two times with PBS and are permeabilized with 0.1% Triton X-100 in PBS for 30 minutes at RT. Next, cells are blocked for 90 min at RT with 20% horse serum, 0.1% Triton X-100 in PBS.

Then, samples are incubated with the primary antibody against Beta Tubulin Isotype III at 4° C. overnight.

Next day, cells are further incubated for another 30 min at RT. After three washing steps with PBS, cells are incubated with a fluorescently labelled secondary antibody and DAPI (nucleus) for 1.5 hours at RT in the darkness. Cells are again rinsed four times with PBS and imaged with the Cytation 5 Multimode reader (BioTek). From each well, images are taken at 10× magnification.

Digital images from cortical neurons are analyzed for the following parameter using a software-supported automatic quantification method: Number of neurites, number of branches, total length of neurites and length of the longest neurite. Analysis is performed using HCA-Vision software or similar standard software.

Basic statistical analysis is performed. If appropriate, data are presented as mean±standard error of mean (SEM) and group differences are evaluated by e.g. one or two-way ANOVA or T-test. EC50 may be calculated as described elsewhere.

### EXAMPLE 61: Agonist Effects at the Human Trace Amine Associated Receptor (hTAAR1)

hTAAR1 was measured with the DiscoverX system in cAMP Hunter™ Gs cell lines overexpress naturally Gs coupled, wild type GPCRs and are designed to detect increases in intracellular cAMP levels in response to agonist stimulation of the receptor in conjunction with the HitHunter® cAMP Assay Detection Kit. Percentage activity was calculated using the following formula:

% Activity=100%×(1−(mean RLU of test sample−mean RLU of MAX control)/(me an RLU of vehicle control−mean RLU of MAX control)).

p-Tyramine was used as a positive control and had an EC50 of 1.137683 μM and a Hill constant of 0.7453. In contrast, S-6-MBPB and R-6-MBPB were measured at seven concentrations between 0.05692 and 60 μM and unexpectedly showed no agonist activity. Maximum detected effects were 0.6% and 0.9%, respectively, for S-6-MBPB and R-6-MBPB, which were considered noise fluctuations.

While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

We claim:

1. A crystalline compound of structure: 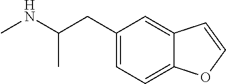
which is an HCl salt and is characterized by an X-ray powder diffraction (XRPD) pattern comprising at least four 2theta values selected from 13.2, 15.1, 16.9, 17.9, 19.2, 20.8, 22.5, 24.4, 25.4, 25.8, 26.2, 27.6, and 31.6±0.4° 2theta.

2. The crystalline compound of claim 1, wherein the XRPD pattern comprises at least six 2theta values selected from 13.2, 15.1, 16.9, 17.9, 19.2, 20.8, 22.5, 24.4, 25.4, 25.8, 26.2, 27.6, and 31.6±0.4° 2theta.

3. The crystalline compound of claim 1, wherein the XRPD pattern comprises at least the 2theta value of 19.2±0.4°.

4. The crystalline compound of claim 1, wherein the XRPD pattern comprises at least the 2theta value of 27.6±0.4°.

5. The crystalline compound of claim 1, wherein the XRPD pattern comprises at least the 2theta value of 15.1±0.4°.

6. The crystalline compound of claim 1, wherein the XRPD pattern comprises at least the 2theta value of 17.9±0.4°.

7. The crystalline compound of claim 1, wherein the XRPD pattern comprises at least the 2theta value of 25.4±0.4°.

8. The crystalline compound of claim 1, wherein the XRPD pattern comprises at least the 2theta value of 22.5±0.4°.

9. A crystalline compound of structure: 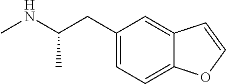
which is an HCl salt and is characterized by an X-ray powder diffraction (XRPD) pattern comprising at least four 2theta values selected from 6.7, 12.7, 13.4, 15.8, 19.0, 19.6, 21.2, 24.7, 25.1, 26.1, 26.8, 28.1, 29.0, 30.4, 31.1, and 39.7±0.4° 2theta.

10. The crystalline compound of claim 9, wherein the XRPD pattern comprises at least six 2theta values selected from 6.7, 12.7, 13.4, 15.8, 19.0, 19.6, 21.2, 24.7, 25.1, 26.1, 26.8, 28.1, 29.0, 30.4, 31.1, and 39.7±0.4° 2theta.

11. The crystalline compound of claim 9, wherein the XRPD pattern comprises at least the 2theta value of 26.8±0.4°.

12. The crystalline compound of claim 9, wherein the XRPD pattern comprises at least the 2theta value of 19.0±0.4°.

13. The crystalline compound of claim 9, wherein the XRPD pattern comprises at least the 2theta value of 24.7±0.4°.

14. The crystalline compound of claim 9, wherein the XRPD pattern comprises at least the 2theta value of 25.1±0.4°.

15. The crystalline compound of claim 9, wherein the XRPD pattern comprises at least the 2theta value of 13.4±0.4°.

16. The crystalline compound of claim 9, wherein the XRPD pattern comprises at least the 2theta value of 6.7±0.4°.

17. A method of treating a central nervous system disorder comprising administering to a host in need thereof, an effective amount of a crystalline compound of structure: 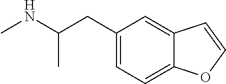
which is an HCl salt and is characterized by an X-ray powder diffraction (XRPD) pattern comprising at least four 2theta values selected from 13.2, 15.1, 16.9, 17.9, 19.2, 20.8, 22.5, 24.4, 25.4, 25.8, 26.2, 27.6, and 31.6±0.4° 2theta;
or an effective amount of a crystalline compound of structure: 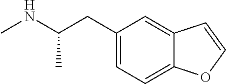
which is an HCl salt and is characterized by an X-ray powder diffraction (XRPD) pattern comprising at least four 2theta values selected from 6.7, 12.7, 13.4, 15.8, 19.0, 19.6, 21.2, 24.7, 25.1, 26.1, 26.8, 28.1, 29.0, 30.4, 31.1, and 39.7±0.4° 2theta.

18. The method of claim 17, wherein the crystalline compound is a racemic mixture of structure: 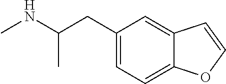
which is an HCl salt and is characterized by an XRPD pattern comprising at least six 2theta values selected from 13.2, 15.1, 16.9, 17.9, 19.2, 20.8, 22.5, 24.4, 25.4, 25.8, 26.2, 27.6, and 31.6±0.4° 2theta.

19. The method of claim 17, wherein the crystalline compound of structure 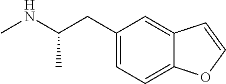
which is an HCl salt and is characterized by an XRPD pattern comprising at least six 2theta values selected from 6.7, 12.7, 13.4, 15.8, 19.0, 19.6, 21.2, 24.7, 25.1, 26.1, 26.8, 28.1, 29.0, 30.4, 31.1, and 39.7±0.4° 2theta.
